(19) United States
(12) Patent Application Publication
(10) Pub. No.: US 2025/0313857 A1
(43) Pub. Date: Oct. 9, 2025

(54) ENPP1 GENE THERAPY FOR THE TREATMENT OF VASCULAR DISEASE

(71) Applicants: The General Hospital Corporation, Boston, MA (US); The Brigham and Women's Hospital, Inc., Boston, MA (US)

(72) Inventors: Rajeev Malhotra, Boston, MA (US); Patricia L. Musolino, Boston, MA (US); Mark Evan Lindsay, Winchester, MA (US); Natalie Artzi, Brookline, MA (US); Eliz Amar-Lewis, Newton, MA (US); Christian Lacks Lino Cardenas, Boston, MA (US)

(21) Appl. No.: 19/171,179

(22) Filed: Apr. 4, 2025

Related U.S. Application Data
(60) Provisional application No. 63/574,833, filed on Apr. 4, 2024.
(60) Provisional application No. 63/574,782, filed on Apr. 4, 2024.
(60) Provisional application No. 63/719,040, filed on Nov. 11, 2024.
(60) Provisional application No. 63/719,028, filed on Nov. 11, 2024.

(51) Int. Cl. C12N 15/86; C12N 9/14

(52) U.S. Cl. CPC C12N 15/86; C12N 9/14; C12N 2750/14143

(57) ABSTRACT

Provided herein are compositions and methods for gene therapy for disorders of arterial calcification as well as Generalized Arterial Calcification of Infancy (GACI). The methods include a gene addition strategy to deliver a DNA construct to target tissues (such as liver and smooth muscle cells) to express soluble recombinant ENPP1 (srENPP1) or transmembrane full-length recombinant ENPP1 (rENPP1).

### CLAIM OF PRIORITY

This application claims the benefit of U.S. Provisional Patent Application Ser, Nos. 63/574,782, filed on Apr. 4, 2024, 63/574,833, filed on Apr. 4, 2024, 63/719,028, filed on Nov. 11, 2024, and 63/719,040, filed on Nov. 11, 2024. The entire contents of the foregoing are hereby incorporated by reference herein.

### SEQUENCE LISTING

This application contains a Sequence Listing that has been submitted electronically as an XML file named 29539-0765001_SL_ST26.xml. The XML file, created on Apr. 4, 2025, is 321,155 bytes in size. The material in the XML file is hereby incorporated by reference in its entirety.

### TECHNICAL FIELD

Provided herein are compositions and methods for gene therapy for disorders of arterial calcification as well as generalized arterial calcification of infancy (GACI). The methods include a gene addition strategy to deliver a DNA construct to target tissues (such as liver and smooth muscle cells) to express soluble recombinant ENPP1 (srENPP1) or transmembrane domain-containing (e.g., full-length) recombinant ENPP1 (rENPP1).

### BACKGROUND

Generalized arterial calcification of infancy (GACI) is a rare genetic disorder that affects the circulatory system in general, and the large and medium sized arteries in particular, throughout the body. GACI is caused by autosomal recessive mutations in the ectonucleotide pyrophosphatase/phosphodiesterase 1 (ENPP1) or ABCC6 genes (Nitschke et al., Am J Hum Genet. 2012 Jan. 13; 90(1):25-39; Ziegler et al., Generalized Arterial Calcification of Infancy. In: GeneReviews® [Internet]. Seattle (WA): University of Washington, Seattle; 1993. 2014 Nov. 13 [updated 2020 Dec. 30]). The disease affects children and young adults, and diagnosis can be made in utero or the first months of life. Infants present with heart disease (e.g., myocardial infarction, heart failure, valvular disease, and/or ischemic cardiomyopathy), and the highest mortality rates occur in the first 6 months of life, with about 55% of affected infants dying. During the course of the disease, the arteries mineralize (calcification) and narrow (intima proliferation) causing myocardial infarction, heart failure, kidney failure, severe hypertension, and strokes. Patients who survive to later childhood and adult life typically have phosphate wasting leading to hearing loss, rickets/ostemalacia (e.g., autosomal recessive hypophosphatemic rickets Type 2 (ARHR2), skin findings, and vision loss; see, e.g., Rutsch et al., Circ Cardiovasc Genet. 2008 December; 1(2):133-40).

Although GACI is an extreme example, other conditions are associated with similar vascular calcification pathology, including pseudoxanthoma elasticum (PXE), calciphylaxis, and cardiovascular diseases including diabetic vascular calcification, end-stage renal disease (ESRD)-associated vascular disease, calcific aortic valve disease (CAVD), coronary atherosclerosis, coronary artery bypass graft (CABG) stenosis, in-stent stenosis, peripheral vascular disease, and cerebral atherosclerosis.

### SUMMARY

Provided herein are constructs for expression of ectonucleotide pyrophosphatase/phosphodiesterase 1 (ENPP1), comprising a sequence encoding an ENPP1 transgene, wherein the ENPP1 is (i) full-length human ENPP1 (also referred to herein as rENPP1) or (ii) a truncated version thereof comprising the extracellular soluble domain of human ENPP1 (srENPP1) linked to a stabilizing protein, and a promoter that drives expression of the ENPP1 transgene. In some embodiments, the sequence encoding the ENPP1 transgene is codon optimized for expression in human cells. In some embodiments, the constructs comprise (from 5′ to 3′) a promoter, an optional spacer sequence of about 10-100 or 30-100 nts, a kozak sequence, a secretion signal sequence, a sequence encoding an srENPP1 transgene, a stabilizing protein, optionally with a linker (optionally 10-20 nt long, e.g., comprising CTGATCGTTAAC (SEQ ID NO:104)) between the ENPP1 protein and stabilizing protein, a polyadenylation sequence, and optionally one or more copies of one or more miRNA target sequences, e.g., three copies of a mir155 and/or miR122 target sequence (e.g., comprising one or more repeats of the sequence CAATTACGATTAGCACTATC (SEQ ID NO:2), optionally comprising the sequence CAATTACGATTAGCACTATCCAATTACGATTAGCACTATCCAATTACGATTAGCAC TATC (SEQ ID NO:3), or comprising one or more repeats, e.g., three repeats, of the sequence ACAAACACCATTGTCACACTCCA (SEQ ID NO:106)).

Additionally provided herein are constructs for expression of ectonucleotide pyrophosphatase/phosphodiesterase 1 (ENPP1) comprising a sequence encoding an ENPP1 transgene, wherein the ENPP1 is (i) full-length human ENPP1 or (ii) a truncated version thereof comprising the extracellular domain of human ENPP1 (srENPP1) linked to a stabilizing protein, and a promoter that drives expression of the ENPP1 transgene, wherein the construct is packaged in an adeno-associated virus (AAV), preferably wherein the AAV is AAV9 comprising a modified capsid, preferably wherein a VP1 protein of the modified capsid comprises the sequence PRPPSTH (SEQ ID NO:44), MAEPGAR (SEQ ID NO:45), MLYADNT (SEQ ID NO:46), or SQDPSTL (SEQ ID NO:47) inserted into the VP1 protein in a position corresponding to between amino acids 588 and 589.

Also provided herein are nucleic acid constructs for expression of an ENPP1 enzyme, comprising: a nucleic acid sequence encoding an AAV genome comprising: a first inverted terminal repeats (ITR); a nucleic acid encoding a replication (rep) sequence; a nucleic acid encoding a capsid (cap) sequence, the nucleic acid encoding the cap sequence comprising a sequence encoding the peptide PRPPSTH inserted therein (SEQ ID NO:44); a promoter; and a nucleic acid encoding an ectonucleotide pyrophosphatase/phosphodiesterase 1 (ENPP1) enzyme comprising a transmembrane domain; and a second ITR.

In some embodiments, the constructs further comprise one or more sequences that promote expression of the ENPP1 transgene, optionally one or more enhancer sequences (e.g., 5′ untranslated region (UTR) or a 3′ UTR) and/or insulator sequences.

In some embodiments, the constructs comprise one or more regulatory sequences, e.g., tandem repeats of one or more microRNA (miRNA) target sites incorporated into 3′ UTR, optionally wherein the one or more miRNA target sites are selected from miRNA 155 (miR155), miR21, miR122, miR210, miR30b, miR103, and/or miR82 target sites. In some embodiments, the constructs comprise at least two or three tandem repeats of a miR155 target site comprising the sequence CAATTACGATTAGCACTATC (SEQ ID NO:2) or at least two or three tandem repeats of a miR122 target site comprising the sequence ACAAACACCATTGTCACACTCCA (SEQ ID NO:106).

In some embodiments, the constructs further comprise a woodchuck hepatitis virus posttranscriptional response element (WPRE).

In some embodiments, the promoter is CMV immediate/early gene enhancer/CBA promoter (CAG); cytomegalovirus (CMV) promoter, chicken beta-actin (CBA) promoter, Rous sarcoma virus (RSV) LTR promoter, SV40 promoter, dihydrofolate reductase promoter, phosphoglycerol kinase promoter, phosphoglycerol kinase (PGK) promoter, EF1alpha promoter, Ubiquitin C (UBC), B-glucuronidase (GUSB), hAlb, hMGP, or HDAC9_prom2 HDAC9 promoter, Hepcidin promoter, Myh11 promoter, ENPP1 promoter, ENPP2 promoter, or ENPP3 promoter.

In some embodiments, the stabilizing protein is human albumin, transthyretin, transferrin, or IgG Fc; in some embodiments, the stabilizing protein is not IgG Fc.

In some embodiments, the constructs comprise srENPP1 linked to human albumin, optionally wherein srENPP1 is codon-optimized and human albumin is not.

In some embodiments, the constructs comprise a viral vector, e.g., an adeno-associated virus (AAV), optionally AAVPR.

In some embodiments, the constructs comprise a sequence that is at least 80%, 85%, 90%, 95%, 97%, 98%, or 99% identical to a construct shown herein, preferably omitting any tag sequences or plasmid sequences.

In some embodiments, the constructs comprise a construct as described herein, e.g., listed in Table B, or having a sequence provided herein, optionally omitting any tag.

Also provided herein are pharmaceutically acceptable compositions comprising the constructs described herein.

Additionally provided herein are methods of treating a subject who has a condition associated with vascular calcification, the method comprising administering to the subject a therapeutically effective amount of a construct or composition as described herein. In some embodiments, the condition is generalized arterial calcification of infancy (GACI), pseudoxanthoma elasticum (PXE), calciphylaxis, and cardiovascular disease including diabetic vascular calcification, end-stage renal disease (ESRD)-associated vascular disease, calcific aortic valve disease (CAVD), coronary atherosclerosis, coronary artery bypass graft (CABG) stenosis, in-stent stenosis, peripheral vascular disease, or cerebral atherosclerosis.

In some embodiments, the construct is administered intravenously.

Also provided herein are particles comprising:
a) an amount of an ionizable lipid;
   an amount of neutral lipid;
   an amount of cholesterol;
   an amount of one or more PEG-lipids; and
   an amount of a DOTAP molecule;
b) a peptide conjugated to a linker in the particle; and
c) a construct for expression of ectonucleotide pyrophosphatase/phosphodiesterase 1 (ENPP1), comprising a sequence encoding an ENPP1 transgene, wherein the ENPP1 is (i) full-length human ENPP1 or (ii) a truncated version thereof comprising the extracellular domain of human ENPP1 (srENPP1) linked to a stabilizing protein, and a promoter that drives expression of the ENPP1 transgene.

In some embodiments, the linker is a maleimide group at a PEG lipid of the one or more PEG-lipids in the particle.

In some embodiments, at least one of the one or more PEG-lipids is a maleimide-terminally modified PEG lipid.

In some embodiments, the one or more PEG-lipids comprise DMG-PEG and/or DSPE-PEG-maleimide.

In some embodiments, the peptide is a peptide targeting collagen IV (Col-IV), IL-6R, CD63, GAL-3, or any combination thereof.

In some embodiments, the ionizable lipid, the neutral lipid, the cholesterol, the one or more PEG-lipids, and DOTAP are present at a molar ratio of 10:2.1:7.6:1.5:78.8.

In some embodiments, the phosphatidylcholine lipid or the phosphatidylethanolamine lipid is selected from the group consisting of DOPE, DOPC, DSPC, DPPC, POPC, and SOPC.

In some embodiments, the ionizable lipid is selected from the group consisting of DLin-MC2-DMA, DLin-MC3-DMA, DSDMA, DODMA, DLinDMA, DLenDMA, 7-DLenDMA, DLin-K-DMA, DLin-C2K-DMA, DLin-K-C3-DM A, DLin-K-C4-DMA, DLen-C2K-DMA, γ-DLen-C2K-DMA, or DLin-MP-DMA.

In some embodiments, the particles comprise: about 75-85% of DOTAP; about 10% of an MC3 ionizable lipid; about 2-2.5% of a DOPE neutral lipid; about 7-8% of cholesterol; and about 1-2% of one or more PEG-lipids. In some embodiments, the particles comprise: about 78.8% of DOTAP; about 10% of an MC3 ionizable lipid; about 2.1% of a DOPE neutral lipid; about 7.6% of cholesterol; and about 1.5% of one or more PEG-lipids.

Further, provided herein are particles comprising:
from 0.1% to 10% of a molecule of formula I

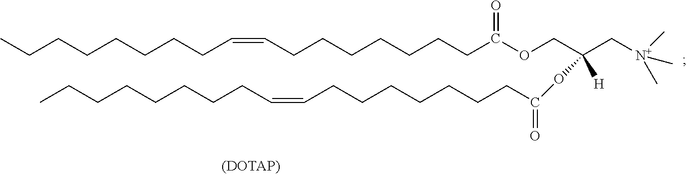 [SMILES: CCCCCCCC/C=C\CCCCCCCC(=O)OC[C@H](C[N+](C)(C)C)OC(=O)CCCCCCC/C=C\CCCCCCCC]

and
a construct as described herein.

In some embodiments, the particles further comprise:
an amount of an ionizable lipid;
an amount of neutral lipid;
an amount of cholesterol;
an amount of a PEG-lipid;
wherein the ionizable lipid, the neutral lipid, the cholesterol, and the PEG-lipid are present at a molar ratio of 50/10/38.5/1.5 of the remaining weight of the particle.

In some embodiments, the particles comprise:
from 45% up to 52% of an ionizable lipid;
from 9% up to 11% of a neutral lipid;
from 34% up to 40% of cholesterol;
from 1% up to 2% of PEG-lipid.

In some embodiments, the PEG-lipid is a maleimide-terminally modified PEG lipid.

In some embodiments, the particles further comprise a peptide conjugated to the particle via the maleimide-terminally modified PEG lipid. In some embodiments, the particles comprise one or more peptides targeting collagen IV (Col-IV), IL-6R, CD63, GAL-3, or any combination thereof.

In some embodiments, the neutral lipid is a phosphatidylcholine lipid or a phosphatidylethanolamine lipid. In some embodiments, the phosphatidylcholine lipid or the phosphatidylethanolamine lipid is selected from the group consisting of DSPC, DPPC, and POPC.

In some embodiments, the ionizable lipid is selected from the group consisting of DLin-MC2-DMA, DLin-MC3-DMA, DSDMA, DODMA, DLinDMA, DLenDMA, γ-DLenDMA, DLin-K-DMA, DLin-C2K-DMA, DLin-K-C3-DM A, DLin-K-C4-DMA, DLen-C2K-DMA, γ-DLen-C2K-DMA, or DLin-MP-DMA.

In some embodiments, the particle comprises:
about 7% of DOTAP;
about 46% of an MC3 ionizable lipid;
about 9.8% of a DOPE neutral lipid;
about 35.3% of cholesterol; and
about 1.4% a PEG-lipid.

Also provided herein are pharmaceutically acceptable compositions or therapeutic formulations comprising particles as described herein.

Additionally, provide herein are methods of delivering an ENPP1 nucleic acid therapeutic cargo to a smooth muscle cell; the methods comprise administering to or contacting the smooth muscle cell with a construct, composition, particle, or formulation as described herein.

Further, provided herein are methods of treating a subject who has a condition associated with vascular calcification, comprising administering to the subject a therapeutically effective amount of a particle, composition, or therapeutic formulation as described herein. Also provided are the constructs, particles, compositions, or therapeutic formulations for use in a method of treating a subject who has a condition associated with vascular calcification. In some embodiments, the condition is generalized arterial calcification of infancy (GACI), pseudoxanthoma elasticum (PXE), calciphylaxis, and cardiovascular disease including diabetic vascular calcification, ESRD-associated vascular disease, calcific aortic valve disease (CAVD), coronary atherosclerosis, coronary artery bypass graft (CABG) stenosis, in-stent stenosis, peripheral vascular disease, or cerebral atherosclerosis.

As used herein, “about” means plus or minus 10%, unless otherwise indicated. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-B. Schematic illustrations of two general exemplary methods for delivery of ENPP1 gene therapy for systemic gene therapy, e.g., for infantile GACI, as described herein. 1A, Gene therapy using a construct encoding a soluble secreted recombinant ENPP1 (srENPP1) protein, delivered to hepatocytes; in this method, the protein is produced in the liver and secreted into the bloodstream. 1, Gene therapy using a construct encoding a transmembrane full-length recombinant ENPP1 (rENPP1), delivered to hepatocytes and vascular smooth muscle cells (VSMCs); in this method, the protein is produced locally in the vascular tissues as well as in the liver.

FIG. 2. Schematic illustration of four DNA constructs with the CBA or CMV promoter driving expression for delivering a soluble secreted recombinant srENPP1:

CBA_srENPP1_Albumin—contains (1) albumin secretory signal (hAlb sig) to signal the protein for secretion from the cell and (2) hAlbumin to stabilize the protein and increase its half-life;

CBA_srENPP1—contains the albumin secretory signal but not a stabilizing fusion protein;

CBA_srENPP1_Fc—contains ENPP1 cytosolic domain (CD, amino acids 1-76), ENPP2 signal sequence (SS, amino acids 12-30), with the extracellular ENPP1 domain fused with the IgG Fc domain for stabilization;

CMV_srENPP1_Albumin—contains (1) albumin secretory signal (hAlb sig) to signal the protein for secretion from the cell and (2) hAlbumin to stabilize the protein and increase its half-life.

Most of the constructs also included Kozak sequences and spacer sequences from the pcDNA3.1 plasmid between the Kozak sequences and the promoter. The promoter can be swapped, e.g., CMV replaced with CBA and vice-versa.

FIG. 3. Schematic illustration of DNA constructs with the CBA (top) or CMV (bottom) promoter driving expression of a FLAG-tagged human recombinant transmembrane full-length ENPP1 (rENPP1).

FIG. 4. Western blot analysis of ENPP1 constructs under the CBA or CMV promoters from total lysates of human liver cells (HepG2). In lanes A and C, results showed superior expression of the srENPP1 transgene (160 kD) in cells transfected with the CMV_srENPP1_albumin vs CBA_srENPP1_albumin as assessed by ENPP1 (top blot) or Flag-tag (middle blot) antibody immunoblot. In lanes B and D, results showed superior expression of the transmembrane rENPP1 transgene (130 kD) in cells transfected with the CMV_rENPP1 vs CBA_rENPP1.

### Lanes:

A. CBA_srENPP1_Albumin
B. CBA_rENPP1
C. CMV_srENPP1_Albumin
D. CMV_rENPP1
Ctrl. Untransfected
Expected molecular weights: Soluble srENPP1=160 kD; Transmembrane rENPP1=130 kD

FIG. 5. Western blot analysis of rENPP1 constructs under the CBA or CMV promoters from total lysates of human cells HEK293T. In lanes A and C, results showed superior expression of the srENPP1 transgene (160 kD) in cells transfected with the CMV_srENPP1_albumin vs CBA_srENPP1_albumin as assessed by ENPP1 (top blot) or Flag-tag (middle blot) antibody immunoblot. In lanes B and D, results showed superior expression of the transmembrane rENPP1 transgene (130 kD) in cells transfected with the CMV_rENPP1 vs CBA_rENPP1.

### Lanes:

A. CBA_srENPP1_Albumin
B. CBA_rENPP1
C. CMV_srENPP1_Albumin
D. CMV_rENPP1
Ctrl. Untransfected
Expected molecular weights: Soluble srENPP1=160 kD; Transmembrane rENPP1=130 kD

FIG. 6. HepG2 cells (left) and HEK293T cells (right) were co-transfected with the CMV_srENPP1_Albumin plasmid and a control plasmid (pcDNA3-mRFP) and cultured in minimal essential media with FBS. RFP expression was confirmed under microscope 3 days after transfection. Cell supernatants were harvested for western blot analysis at 72 h post transfection. Supernatant protein was pulled down with anti-FLAG antibody and probed for either ENPP1 or FLAG. Recombinant Human ENPP1/PC1 protein (ab167943) was used as a positive control. These results indicate good cellular secretion of srENPP1 into the supernatant of human cells transfected with CMV_srENPP1_Albumin.

FIGS. 7A-B. srENPP1 was enzymatically active in the HepG2/HEK293T cell supernatants. Enzymatic phosphodiesterase activity of srENPP1 was measured from the supernatants of HepG2 cells (7A) and HEK293T cells (7B) transfected with plasmid expressing srENPP1 or control cells. A significant increase in the ENPP1 activity (right panels in both the figures) was observed compared to supernatants from untransfected cells. Wild-type mouse liver (“Asj+/+ liver”) was used as a positive control and cell media without cells was used as a negative control.

FIGS. 8A-B. Construct for soluble recombinant ENPP1 linked to Fc (srENPP1-Fc) was enzymatically active in HepG2 cell lysates but not the supernatants. Enzymatic phosphodiesterase activity of srENPP1-Fc (“plasmid #4”) was measured from the cell lysates (8A) and supernatants (8B) of HepG2 cells transfected with plasmid #4. A significant increase in the ENPP1 activity was observed compared to cell lysates from untransfected cells but no increase in activity was observed in the supernatant of transfected cells, indicating that this soluble version of the ENPP1 is not appropriately secreted from cells.

FIGS. 9A-B. CMV_srENPP1_hAlbumin gene delivery using LTX lipofectamine and 4 versions of nanoparticles to assess srENPP1 expression in cell lysates (9A) or supernatant (9B) from human HepG2 cells. Expression was detected in cell lysates using a Flag-directed antibody (9A) and in supernatants using an ENPP1 antibody and Flag antibody (9B). Results demonstrated srENPP1 transgene expression levels for CHK18 and CHK30 nanoparticles comparable to commercial LTX lipofectamine reagent using either a Flag-directed antibody or an ENPP1-directed antibody.

FIG. 10. ENPP1 enzymatic activity assay from the supernatant of human HepG2 cells transfected with CMV_srENPP1_albumin using nanoparticles. Right panel indicates absolute values of increased ENPP1 activity of cells transfected with CMV_srENPP1_albumin compared with untransfected cells. Results show transgene delivery and activity in CHK18 nanoparticles comparable with LTX lipofectamine.

FIG. 11. ENPP1 enzymatic activity assay from total lysates of human HepG2 cells transfected with CMV_srENPP1_albumin using nanoparticles. Right panel indicates absolute values of increased ENPP1 activity of cells transfected with CMV_srENPP1_albumin compared with untransfected cells. Results show transgene delivery and activity with CHK18 nanoparticles that was even higher than with LTX lipofectamine.

FIGS. 12A-B. Graphs representing the fold increase in expression of srENPP1 (12A) and rENPP1 (12B) in different tissues of mice injected with different CMV_srENPP1_Albumin (12A, “plasmid 5”) or CMV_rENPP1 (12B, “plasmid 6”) plasmid concentrations and nanoparticle preparations compared to control un-injected. Newborn pups were injected on day 3 with CMV_srENPP1_Albumin expression plasmid packed in various nanoparticle preparations through retroorbital route. Tissue/organs were harvested on day 6 post injection and placed on fresh OCT blocks followed by cryosectioning and immunohistochemistry to study the expression profile of srENPP1 or rENPP1 in various tissues and organs. Depicted are the quantification of the immunohistochemistry signals from anti-FLAG antibody representing transgene expression.

FIGS. 13A-F. In vitro assessment of AAVPR rENPP1 expression, activity, and inhibition of osteogenic VSMC phenotypic change. (A) AAVPR-GFP transduces human VSMCs more robustly than AAV9-GFP. (B) Human aortic VSMCs treated with AAVPR rENPP1 at low and high doses demonstrated dose-dependent overexpression of ENPP1 at the protein level. (C) Increased rENPP1 activity was detected in human VSMCs treated with AAVPR rENNP1 compared to untreated cells. AAVPR rENPP1 treatment decreased VSMC osteogenic phenotype switch: (D) AAVPR rENPP1 treated cells demonstrated decreased calcification on Alizarin red staining compared to untreated cells after 21 days of culture in osteogenic media, (E) AAVPR treatment decreased migration of VSMCs compared to control-treated cells after 12 hours in osteogenic media. (F) These phenotypic changes correlated with a reduction in RUNX2 protein levels with AAVPR rENPP1 treatment.

FIG. 13G. ENPP1 gene therapy using LNP delivery in GACI Mice (Asj−/− mice). Compared to vehicle-treated GACI mice, GACI mice treated with poly-beta amino ester (pBAE) srENPP1 or LNP1-DOPE srENPP1 showed improved survival with log rank p=0.03 and p=0.008, respectively.

FIGS. 14A-B. (A) Expression of rENPP1 from CAG-rENPP1-FLAG-miR 122 AND CAG-rENPP1-FLAG-miR 155 plasmids. Flag-tagged rENPP1 was expressed from plasmid with constructs consisting of a CAG promoter upstream of rENPP1-FLAG. One plasmid had miR122 target sequences at the 3′ UTR of the construct (#3-4) and the other plasmid (#5-6) had miR155 target sequences at the 3′ UTR of the construct. Human aortic SMCs were transfected with these plasmids with lipofectamine (LTX). The Western blots, probing for either FLAG expression (on the left) or ENPP1 expression (on the right), demonstrated cellular rENPP1 protein overexpression with these plasmid transfections, highlighting the feasibility of adding 3′ miR regulatory elements to therapeutic constructs.

### Lanes:

0. Ladder
1. Untreated
2. Only LTX treated
3. LTX+rENPP1-miR122-1 ug/well
4. LTX+rENPP1-miR122-2 ug/well
5. LTX+rENPP1-miR155-1 ug/well
6. LTX+rENPP1-miR155-2 ug/well
7. Positive control for FLAG
8. Positive control for ENPP1

(B) Western blot analysis of expression of srENPP1 in constructs with or without miR155 target sequences in the presence of varying concentrations of miR155. Expression of srENPP1 was reduced with miR155 in a dose-dependent manner. HEK 293T Cells (lanes 4-7) transfected with CMV_srENPP1_Albumin plasmid containing miR155 target sequence showed a reduction in expression of srENPP1 with miR155 in a dose dependent manner compared to cells transfected with CMV_srENPP1_Albumin plasmid without miR155 target sequence (lane3).

### Lanes:

1: Molecular weight marker
2: LTX control with 20 nM miR 155
3: CMV_SrENPP1_Albumin without target sequence−with 20 nM miR 155
4: CMV_SrENPP1_Albumin with miR155 target sequence+10 nM miR 155
5: CMV_SrENPP1_Albumin with miR155 target sequence+20 nM miR 155
6: CMV_SrENPP1_Albumin with miR155 target sequence+40 nM miR 155
7: CMV_SrENPP1_Albumin with miR155 target sequence+80 nM miR 155
8: Flag positive control

FIG. 15. ENPP1 activity was reduced with miR155 in a dose-dependent manner. A decrease in ENPP1 activity was observed with miR155 in a dose dependent manner in supernatant isolated from HEK293T cells transfected with plasmid expressing srENPP1_Albumin with miR155 target sequence and treated with miR155 at a range of concentrations (0, 10, 20, 40, and 80 nM).

FIG. 16. Schematic illustration of exemplary constructs encoding full length ENPP1 (rENPP1, top), or soluble ENPP1 (srENPP1, bottom) with a stabilizing protein, optionally including miRNA target sequences in the 3′ UTR, with exemplary non-limiting promoters, secretory sequences, stabilizing proteins, and miRNAs listed below. Other polyA sequences can also be used, e.g., BGH polyA and variants there.

FIGS. 17A-C: LNPs in vitro screening in human liver hepatocytes (HepG2 cell line) based on formulation composition varying in cholesterol and DOPE content (high, medium, low). (A) is a graph showing the delivery of plasmid expressing RFP, measured by flow cytometry. (B-C) are gel electrophoresis images showing the delivery of plasmid construct expressing soluble ENPP1 (srENPP1) and detection in (B) cell lysates and (C) cell supernatant.

FIG. 18: Microscopy images illustrating efficient soluble ENPP1 expression in the liver among other organs 6 days post injection with a particle of the disclosure in an Asj mouse model for generalized arterial calcification of infancy (GACI). Asj mice injected with soluble srENPP1 plasmid (0.3 mg/kg) at day 3 (P3) express high levels of the enzyme in the liver, 6 days post injection.

FIGS. 19A-D: Efficacy study of LNP delivery using plasmids expressing soluble srENPP1. (A) is a schematic illustration presenting the injection regimen at day 3, 10, and 17 days of age. (B) are survival curves of treated animals, (C) a graph showing animal body weight, and (D) MicroCT scans to detect early development of calcification in treated (LNPs encapsulating soluble ENPP1, 0.3 mg/kg) and untreated animals.

FIGS. 20A-B: (A) is a schematic illustration presenting the LNP four-component system encapsulating plasmid DNA and the addition of a fifth lipid. (B) are graphs showing LNPs' hydrodynamic size, PDI, and zeta potential as a function of % of DOTAP in the formulation.

FIG. 21 is a pair of bar graphs illustrating that introduction of DOTAP lipid into a 4-component formulation enables the delivery of plasmid encoding red fluorescent protein (RFP) to MOVAS (mouse smooth muscle) cell line. MOVAS cells incubated for 48 h with plasmid (2 ug/48 well plate) encoding RFP. Cellular expression was identified and measured using flow cytometry. Left, a graph depicting the % of RFP positive cells as a function of % of DOTAP. Right, a graph depicting the mean fluorescence intensity of RFP expression as a function of % of DOTAP.

FIG. 22: are histological images illustrating that the introduction of DOTAP LNPs enabled the in vivo delivery of plasmid DNA to smooth muscle cells (SMCs) in the aorta. In vivo transduction of transmembrane rENPP1 in SMCs of the aorta using DOTAP LNPs. Asj mouse model at P3 injected systemically with PBS (control)(left column), LNPs (conventional four-component formulation)(middle column) and DOTAP LNPs formulation (7% DOTAP)(right column) at 0.3 mg/kg plasmid dose. Histological images of the aorta in which SMCs (F-actin)(bottom row) colocalize with ENPP1 expression (Flag-tag)(top row).

FIGS. 23A-B: (A) is a schematic illustration presenting the LNP four-component system encapsulating mRNA and the addition of a fifth lipid; (B) are charts showing the LNPs' hydrodynamic size, PDI, and zeta potential as a function of % of DOTAP in the formulation.

FIGS. 24A-D: are graphs illustrating that introduction of DOTAP into the formulation enables the in vitro delivery of mRNA encoding GFP to MOVAS cell line and primary human aortic cells cultured from an ACTA2 patient. (A-B) MOVAS cells incubated for 24 h with DOTAP/LNPs encapsulating mRNA (0.1 ug/48 well plate) encoding GFP. Cellular expression was identified and measured using flow cytometry presenting (A) percentage of cells expressing GFP and (B) GFP fluorescent intensity. (C) MOVAS cell viability, assessed to confirm the treatment toxicity. (D) 10% DOTAP LNPs provided robust delivery to primary human aortic.

FIGS. 25A-E: Cellular uptake study (A) of 10% DOTAP LNPs in comparison to 0% and 100% determined by (B) fluorescent microscopy and (C) flow cytometry. LNPs encapsulated mRNA labeled by Cy5. (D) LNPs utilize Caveolin and Macropinocytosis-mediated uptake into SMCs. SMCs were used in an optimized (10% DOTAP) LNP cellular internalization study and the uptake mechanism was investigated using Cy5-fluorescently labeled LNPs (mRNA Cy5). Uptake was quantified using flow cytometry. (E) LNPs with 10% or more DOTAP also provided excellent delivery of plasmid DNA encoding RFP to MOVAS cells.

FIGS. 26A-D: are experimental results depicting in vivo delivery of Cre-mRNA utilizing LNP and DOTAP LNP and expression of tdTom in a Marfan disease mouse model. Mice at P3 injected with 1 mg/kg mRNA encapsulated in LNPs or DOTAP LNPs. (A) is a chart depicting expression of tdTom in different organs 6 days post injection as a fold-change relative to untreated mice. (B) Histological images of the aorta in which tdTom is expressed in SMCs from DOTAP LNP-injected mice. (C) tdTom expression was identified 7 days post injection by fluorescent imaging in the aorta and demonstrated strong SMC cell-specific signal by immunofluorescent microscopy. (D) Histological images of Cre-mRNA delivery utilizing LNPs with increasing DOTAP content and expression of tdTom in a Marfan disease mouse model. Increasing the % of DOTAP in LNP formulation increases SMC tdTom expression in vivo. Mice at P3 were injected with 1 mg/kg mRNA encapsulated in LNPs formulated with 0, 10, 50 and 80% DOTAP lipid. tdTom expression was identified using immunofluorescence in histological sections of the aorta and localized to SMCs (indicated by α-SMA expression).

FIG. 27: is a schematic illustration presenting the LNPs' conjugation scheme. LNPs are conjugated with the desired peptide following the assembly of the nanoparticle. Peptide conjugation was done in different densities controlled by the percentage of the linker lipid in the LNP formulation that was 0.15-1.2%.

FIG. 28: is a chart depicting the correlation between percentage of linker lipid within peptide-conjugated LNPs, their measured peptide concentration and the resulting calculated number of peptides per LNP.

FIG. 29: is a chart depicting the functional activity of peptide-conjugated LNPs in vitro. MOVAS cells treated (24 h) with LNPs conjugated with Col-IV/IL6R/CD63/Gal3 targeting peptides encapsulating mRNA encoding GFP. Cellular expression was measured using flow cytometry.

FIGS. 30A-B: (A) In vivo ENPP1 aortic SMC expression (with immunofluorescence (IF) using antibody specific for the FLAG-tag) in ENPP1^{asi/asj} mice injected at P3 by SMC-optimized LNPs (10% DOTAP). Aortic SMC expression was identified using IF, 7 days post injection. (B) ENPP1 gene therapy in ENPP1^{asj/asj} mice administered with three doses of SMC optimized LNPs (10% DOTAP) encapsulating plasmid expressing ENPP1. Efficacy was identified by vibrissae calcification imaged by microCT.

FIG. 31. Graphs representing the fold increase in expression of rENPP1 in different tissues from mice injected with a CAG_rENPP1 expression construct packaged in AAVPR. Newborn pups were injected on day 3 with rENPP1 expression construct packaged in AAVPR through retroorbital route. Tissue/organs were harvested on day 7 post injection and placed on fresh OCT blocks followed by cryosectioning and immunohistochemistry to study the expression profile of rENPP1 in various tissues and organs.

FIGS. 32A-E: (A) ENPP1 gene therapy with either NP delivery of srENPP1 or AAVPR delivery of rENPP1 reduced vibrissae calcification on micro-CT. (B-C) Micro-CT images of male Asj−/− mice demonstrated decreased vibrissae calcification with different doses of AAVPR-CAG-rENPP1 (B) and graphs quantifying calcification at 6 and 8 weeks are shown (C). (D-E) Micro-CT images of female Asj^{-/-} mice demonstrated decreased vibrissae calcification with different doses of AAVPR-CAG-rENPP1 (D) and graphs quantifying calcification at 6 and 8 weeks are shown (E).

FIGS. 33A-B: (A) Pyrophosphate is the chemical product of ENPP1 enzymatic activity and is significantly reduced in calcification disorders such as GACI. Serum pyrophosphate (PPi) levels were normalized in GACI mice treated with pBAE srENPP1 or AAVPR rENPP1. (B) Plasma pyrophosphate (PPI) was increased in a dose-dependent manner in ASJ^{-/-} GACI mice that received 3 different doses of AAVPR-CAG-rENPP1 (low=3e12 vg/kg, intermediate=1e13 vg/kg, high=3e13 vg/kg).

FIGS. 34A-B: (A) Kaplan-Meier survival curves of ASJ^{-/-} GACI mice injected with AAVPR-CAG-rENPP1 at different doses at P3 of age (low=3e12 vg/kg, intermediate=1e13 vg/kg, high=3e13 vg/kg). (B) Kaplan-Meier survival curves of ASJ^{-/-} mice injected with AAVPR-CAG-rENPP1 at a low dose (3e12 vg/kg) at P14 of age.

FIG. 35: depicts weights of ASJ^{-/-} mice injected retroorbitally with 3 different doses of AAVPR-CAG-rENPP1 (low=3e12 vg/kg, intermediate=1e13 vg/kg, high=3e13 vg/kg). * indicates p<0.05 comparing untreated vs low dose treated Asj^{-/-} mice. # indicates p<0.05 comparing untreated vs intermediate dose treated Asj^{-/-} mice. Generally, body weights of GACI ASJ^{-/-} mice improved with AAVPR-CAG-rENPP1 therapy.

FIGS. 36A-B: (A) Wild-type (WT) and ASJ^{+/-} (HETs or heterozygotes) were injected retro-orbitally with 3 different doses of AAVPR-CAG-ENPP1 (low=3e12 vg/kg, intermediate=1e13 vg/kg, high=3e13 vg/kg). Survival was similar to what would be expected of untreated WT and HET mice. (B) WT and ASJ HETs were injected retro-orbitally at postnatal day 3 (P3) versus postnatal day 14 (P14) with the low dose of AAVPR-CAG-ENPP1. Survival was similar to what would be expected of untreated WT and HET mice.

FIGS. 37A-E: collectively depict treatment of human vascular smooth muscle cells (VSMCs) with AAVPR-CAG-rENPP1. (A) AAVPR-CAG-rENPP1 treatment at low (8e9 vg/mL) and high (1.6e10 vg/mL) doses resulted in dose-dependent increase in ENPP1-Flag expression as measured on western blot. (B) Similarly, AAVPR-CAG-rENPP1 treatment at low and high dose resulted in dose-dependent increase in ENPP1 activity compared to untreated cells. (C) AAVPR-CAG-rENPP1 treatment of human VSMCs grown in osteogenic media for 21 days resulted in reduced calcification on alizarin red stain, and (D) RUNX2 protein levels. RUNX2 is a master transcriptional regulator of the osteogenic phenotype switch of VSMCs that results in calcification across a whole host of disorders, including calciphylaxis, atherosclerosis, calcification related to chronic kidney disease and diabetes mellitus. (E) The osteogenic phenotype switch of VSMCs is associated with increased migration, which was reduced after treatment with AAVPR-CAG-rENPP1.

FIGS. 38A-B. Testing performance of AAVPR in human vessels ex vivo. (A) Surgical Specimens isolated were vessels from patients undergoing lower limb amputation under an IRB-approved protocol. (B) Schematic illustrating perfusion bioreactor, the experimental setup in which human vessels harvested from the operating room were attached to a perfusion pump for AAVPR or AAV9 treatment.

FIGS. 39A-B. AAV-PR has High Transduction Efficiency in Healthy Human Vessels. (A) Illustrative image of a cross-section of a human vessel (A) upon which copies of vector genomes were quantified (right). (B) Fluorescence images of GFP expression after perfusion with AAVPR-GFP construct for one hour demonstrates strong AAVPR transduction of human arteries.

FIGS. 40A-B. Ex vivo Perfused Human Vessels—Comparison of AAVPR-GFP and AAV9-GFP transduction efficiency. (A) The AAVPR-transduced vessels exhibited significant GFP signal in the medial layer with co-localization with α-SMA indicating strong transduction of vascular smooth muscle cells. (B) Ex vivo perfusion of diseased human vessels with AAVPR-CBA-GFP resulted in significantly higher viral copy numbers and GFP expression compared to AAV9-CBA-GFP indicating a great vascular tropism with AAVPR than AAV9.

### DETAILED DESCRIPTION

Generalized arterial calcification of infancy (GACI) is characterized by widespread arterial calcification and/or stenoses of large and medium-sized vessels resulting in a range of clinical manifestations including myocardial infarction, respiratory distress, hypertension, cardiomegaly, and stroke. GACI is estimated to affect one in 200,000 pregnancies. Mortality is particularly high in early infancy; approximately 55% of patients die within the first 6 months of life despite intensive care and supportive measures. After 6 months of life, the mortality rate is markedly reduced and patients tend to survive, though many still have sequalae from their initial hypoxic insults, and a majority eventually develop hearing loss and hypophosphatemic rickets. GACI typically results from biallelic loss-of-function mutations in ENPP1, which encodes an ectonucleotide pyrophosphatase/phosphodiesterase that converts ATP into AMP and pyrophosphate (PPi), a potent inhibitor of calcification. Loss of ENPP1 activity results in decreased quantity of PPi both locally and systemically, and GACI patients have low plasma and urinary PPi concentrations. AMP inhibits vascular smooth muscle cell proliferation, so loss of ENPP1 directly impact vascular function. Nitschke et al., Exp Mol Med. 2018 October; 50(10): 139. Endogenous ENPP1 is integrated into the plasma membrane with a single transmembrane domain and an active extracellular domain. Borza et al., J Biol Chem. 2022 February; 298(2):101526.

An ENPP1-deficient mouse model (the ‘ages with stiffened joints’ (asj) mouse) has been developed that recapitulates calcification and clinical presentation found in GACI patients, see, e.g., Li et al., Dis Model Mech. 2013 September; 6(5): 1227-1235. The mice typically die between ages of 35-71 days, with a median survival of 58 days. Post-mortem histological examination of heart, aorta, and kidneys showed calcification, and about 40-60% of mice exhibit calcification of these organs on microCT. 100% of the Asj mice develop vibrissae vascular calcification and this is considered the most consistent phenotype of the model. See, e.g., Li et al., supra; Khan et al., Dis Model Mech. 2018 Oct. 8; 11(10):dmm03569; Albright et al., Nat Commun. 2015 Dec. 1:6:10006.

Provided herein are compositions and methods for gene therapy for disorders of arterial calcification as well as Generalized Arterial Calcification of Infancy (GACI) and other disorders as described herein. The methods include a gene addition strategy to deliver a DNA construct to target tissues (such as liver and smooth muscle cells) to express soluble recombinant ENPP1 (srENPP1) or recombinant full length transmembrane ENPP1 (rENPP1), e.g., under the CBA, CMV, CAG or other promoters as described herein. The DNA constructs can include 3′ miRNA target sites to allow for regulation of ENPP1 mRNA levels and therefore ENPP1 protein expression. Also described herein are methods of delivering recombinant ENPP1, e.g., intravenously, to a subject who is deficient in ENPP1 to restore enzyme activity and reverse or reduce risk of disease progression, e.g., using a viral vector such as an adeno-associated virus (AAV) or a nanoparticle (NP) carrying an ENPP1 gene construct, optionally including a CBA, CMV, CAG, or other promoter, e.g., as described herein; or using lipid nanoparticles as described herein. Such methods can be used for treatment of GACI and other conditions associated with similar vascular calcification pathology, including pseudoxanthoma elasticum (PXE), calciphylaxis, and cardiovascular diseases including diabetic vascular calcification, end-stage renal disease (ESRD)-associated vascular disease, calcific aortic valve disease (CAVD), coronary atherosclerosis, coronary artery bypass graft (CABG) stenosis, in-stent stenosis, peripheral vascular disease, and cerebral atherosclerosis.

### Gene Therapy Constructs

Provided herein are gene therapy constructs for expression of ENPP1, e.g., recombinant transmembrane ENPP1 (rENPP1) or secreted recombinant ENPP1 (srENPP1), in cells of a subject. The constructs can thus include sequences encoding full-length human ENPP1 or a truncated version thereof comprising the extracellular domain of human ENPP1 (srENPP1). The constructs can optionally be codon optimized. Exemplary sequences of human ENPP1 protein is provided in GenBank at RefSeq ID NM_006208.3 (nucleic acid) and NP_006199.2 (protein), e.g., as follows:

| (SEQ ID NO: 1) | |
|---|---|
| 1 | merdgcaggg srggeggrap regpagngrd rgrshaaeap gdpqaaasll apmdvgeepl |
| 61 | ekaarartak dpntykvlsl vlsvcvltti lgcifglkps cakevksckg rcfertfgnc |
| 121 | rcdaacvelg nccldyqetc iepehiwtcn kfrcgekrlt rslcacsddc kdkgdcciny |
| 181 | ssvcqgeksw veepcesine pqcpagfetp ptllfsldgf raeylhtwgg llpvisklkk |
| 241 | cgtytknmrp vyptktfpnh ysivtglype shgiidnkmy dpkmnasfsl kskekfnpew |
| 301 | ykgepiwvta kyqglksgtf fwpgsdvein gifpdiykmy ngsvpfeeri lavlqwlqlp |
| 361 | kderphfytl yleepdssgh sygpvssevi kalqrvdgmv gmlmdglkel nlhrclnlil |
| 421 | isdhgmeqgs ckkyiylnky lgdvknikvi ygpaarlrps dvpdkyysfn yegiarnlsc |
| 481 | repnqhfkpy lkhflpkrlh faksdriepl tfyldpqwql alnpserkyc gsgfhgsdnv |
| 541 | fsnmqalfvg ygpgfkhgie adtfenievy nlmcdllnlt papnngthgs lnhllknpvy |
| 601 | tpkhpkevhp lvqcpftrnp rdnlgcscnp silpiedfqt qfnltvaeek iikhetlpyg |
| 661 | rprvlqkent icllsqhqfm sgysqdilmp lwtsytvdrn dsfstedfsn clyqdfripl |
| 721 | spvhkcsfyk nntkvsygfl sppqlnknss giysealltt nivpmyqsfq viwryfhdtl |
| 781 | lrkyaeerng vnvvsgpvfd fdydgrcdsl enlrqkrrvi rnqeilipth ffivltsckd |
| 841 | tsqtplhcen ldtlafilph rtdnsescvh gkhdsswvee llmlhrarit dvehitglsf |
| 901 | yqqrkepvsd ilklkthlpt fsqed |

Amino acids 103-925 are bolded above; in some embodiments, the srENPP1 sequence comprises amino acids 103-925, and preferably amino acids 96-925 or 97-925 of SEQ ID NO:1.

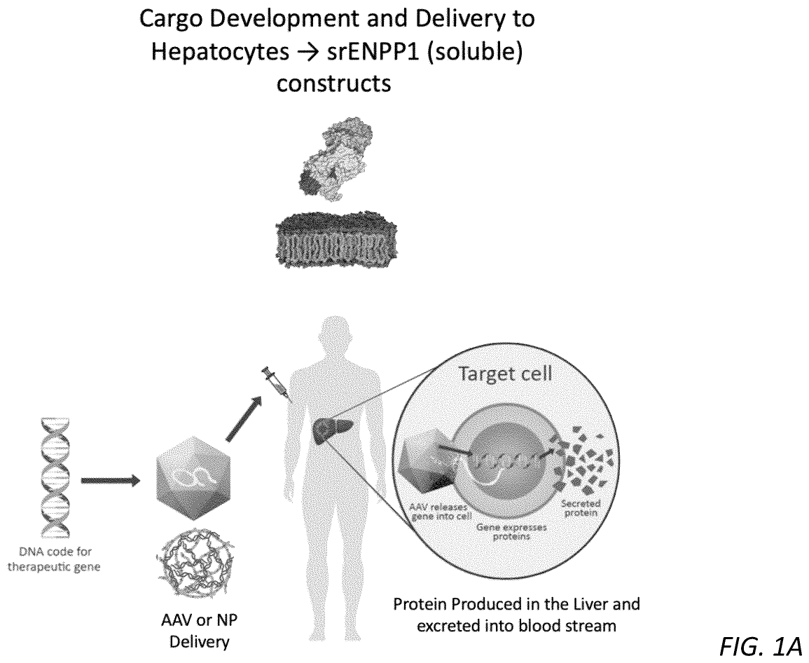

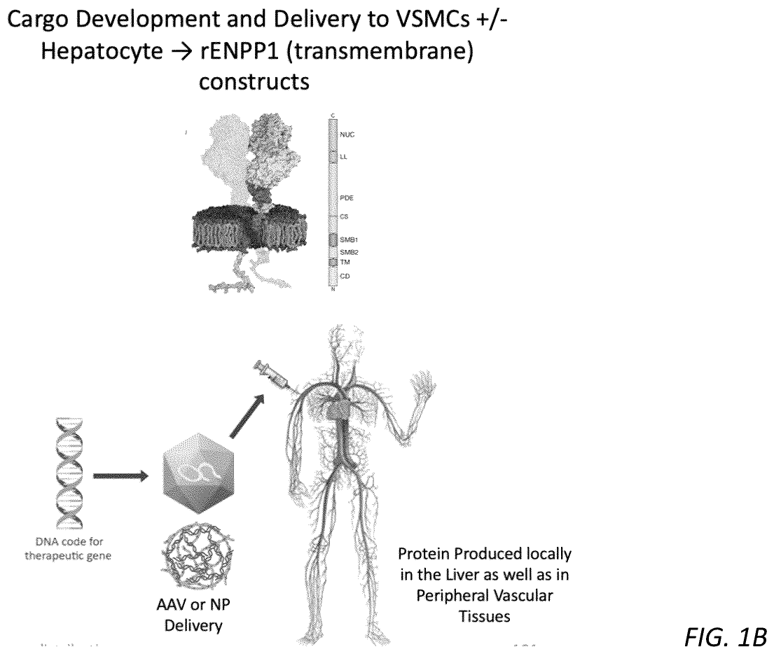

The constructs (examples of which are shown in FIGS. 1A-B, 2, and 3) preferably comprise (from 5′ to 3′) a promoter, an optional spacer sequence (e.g., comprising one or more restriction cut sites to replace promoter or secretory signal-FLAG tag or a sequence, e.g., of about 10-100 or 30-100 nts, from the pcDNA3.1 plasmid providing space between the promoter and Kozak sequence), a kozak sequence, secretion signal sequences for soluble proteins, a transgene sequence comprising ENPP1 sequence (e.g., srENPP1 or rENPP1) and optionally a linker (e.g., between the srENPP1 protein and stabilizing protein) and a stabilizing protein, and a polyadenylation sequence. The construct can also include one or more sequences that promote expression of a transgene, e.g., one or more enhancer sequences, e.g., 5′ untranslated region (UTR) or a 3′ UTR; and/or insulator sequences (see, e.g., Haberman and McCrown, Methods. 2002 October; 28(2):219-26; Suoranta et al., Front Mol Med. 2022 Nov. 1:2:1054069). The woodchuck hepatitis virus posttranscriptional response element (WPRE) can also be used. An exemplary construct can include a Kozak sequence, a human albumin (hAlb) signal sequence, and a sequence encoding a soluble ENPP1-human albumin fusion protein, with a CBA, CMV, CAG, hAlb, hepcidin, ENPP1, ENPP2, ENPP3, myH11, hMGP, or HDAC9 (e.g., HDAC9_prom2 promoter, optionally HDAC9-P2.1 as described herein), and optionally one or more copies of one or more miRNA target sequences, e.g., mir155×3 (e.g., comprising one or more repeats of CAATTACGATTAGCACTATC (SEQ ID NO:2), e.g., comprising the sequence CAATTACGATTAGCACTATCCAATTACGATTAGCACTATCCAATTACGATTAGC ACTATC (SEQ ID NO:3), or another miRNA, e.g., as described herein. Optionally, the sequence encoding the ENPP1 is codon optimized, but the human albumin is wild type (not codon optimized). Another exemplary construct includes a Kozak sequence and a sequence encoding a full length ENPP1, with a CBA, CMV, CAG, hAlb, hepcidin, ENPP1, ENPP2, ENPP3, MYH11, hMGP, or HDAC9 (e.g., HDAC9_prom2 promoter, optionally HDAC9-P2.1 as described herein) promoter. An exemplary spacer sequence is CTCTGGCTAACTAGAGAACCCACTGCTTACTGGCTTATCGAAATTAATACGACT CACTATAGGGAGACCCAAGCTGGCTAGC (SEQ ID NO:4). See, e.g., FIG. 16.

The constructs used in the methods and compositions described herein can include the examples shown in Table B, optionally omitting the tag sequence. Exemplary sequences for the constructs listed in Table B are provided below. The constructs comprising srENPP1 can also include a bone targeting sequence, e.g., at the N- or C-terminus of the protein product, preferably at the C-terminus.

TABLE B
Exemplary constructs and applications
Soluble ENPP1

| | | Sig | | | | 3′ | Disease |
|---|---|---|---|---|---|---|---|
| Promoter | Kozak | Seq | Tag* | ENPP1 | Fusion | UTR | Condition |
| CMV | Kozak | hAlb-ss | FLAG | opt- | hAlb | | GACI, calciphylaxis, PXE |
| | | | | srENPP1 | | | |
| CAG | Kozak | hAlb-ss | FLAG | opt- | hAlb | | GACI, calciphylaxis, PXE, CKD, |
| | | | | srENPP1 | | | diabetic calcification, atherocalcific |
| | | | | | | | disease, aortic valve calcification |
| hAlb | Kozak | hAlb-ss | FLAG | opt- | hAlb | | GACI, calciphylaxis, PXE, CKD, |
| | | | | srENPP1 | | | diabetic calcification, atherocalcific |
| | | | | | | | disease, aortic valve calcification |
| HDAC9_prom2 | Kozak | hAlb-ss | FLAG | opt- | hAlb | | calciphylaxis, CKD, diabetic |
| | | | | srENPP1 | | | calcification, atherocalcific disease |
| CMV | Kozak | hAlb-ss | FLAG | opt- | hAlb | mir155x3 | GACI, calciphylaxis, PXE |
| | | | | srENPP1 | | | |
| hMGP | Kozak | hAlb-ss | FLAG | opt- | hAlb | | GACI, calciphylaxis, PXE, CKD, |
| | | | | srENPP1 | | | diabetic calcification, atherocalcific |
| | | | | | | | disease, aortic valve calcification |
| hMGP | Kozak | hMGP-ss | FLAG | opt- | hAlb | | GACI, calciphylaxis, PXE, CKD, |
| | | | | srENPP1 | | | diabetic calcification, atherocalcific |
| | | | | | | | disease, aortic valve calcification |
| CAG | Kozak | hAlb-ss | FLAG | opt- | hAlb | mir155x3 | GACI, calciphylaxis, PXE, CKD, |
| | | | | srENPP1 | | | diabetic calcification, atherocalcific |
| | | | | | | | disease, aortic valve calcification |
| CMV | Kozak | hAlb-ss | FLAG | opt- | hAlb | mir122x3 | GACI, calciphylaxis, PXE |
| | | | | srENPP1 | | | |
| CAG | Kozak | hAlb-ss | FLAG | opt- | hAlb | mir122x3 | GACI, calciphylaxis, PXE, CKD, |
| | | | | srENPP1 | | | diabetic calcification, atherocalcific |
| | | | | | | | disease, aortic valve calcification |

Transmembrane ENPP1:

| Promoter | Kozak | ENPP1 | Tag* | 3′ UTR | Disease Condition |
|---|---|---|---|---|---|
| CMV | Kozak | rENPP1 | FLAG | | GACI, calciphylaxis, PXE |
| CAG | Kozak | rENPP1 | FLAG | | GACI, calciphylaxis, PXE, CKD, diabetic |
| | | | | | calcification, atherocalcific disease, |
| | | | | | aortic valve calcification |
| CMV | Kozak | rENPP1 | FLAG | mir155x3 | GACI, calciphylaxis, PXE, CKD, diabetic |
| | | | | | calcification, atherocalcific disease, |
| | | | | | aortic valve calcification |
| CAG | Kozak | rENPP1 | FLAG | mir155x3 | GACI, calciphylaxis, PXE, CKD, diabetic |
| | | | | | calcification, atherocalcific disease, |
| | | | | | aortic valve calcification |
| CMV | Kozak | rENPP1 | FLAG | mir122x3 | GACI, calciphylaxis, PXE |
| CAG | Kozak | rENPP1 | FLAG | mir122x3 | GACI, calciphylaxis, PXE, CKD, diabetic |
| | | | | | calcification, atherocalcific disease, |
| | | | | | aortic valve calcification |
| HDAC9_prom2 | Kozak | rENPP1 | FLAG | | calciphylaxis, CKD, diabetic calcification, |
| | | | | | atherocalcific disease |
| HDAC9_prom2 | Kozak | rENPP1 | FLAG | miR-155 | GACI, calciphylaxis, CKD, diabetic |
| | | | | | calcification, atherocalcific disease |
| Myh11 | Kozak | rENPP1 | FLAG | | GACI, calciphylaxis, PXE, CKD, diabetic |
| | | | | | calcification, atherocalcific disease |
| Myh11 | Kozak | rENPP1 | FLAG | miR-122 | GACI, calciphylaxis, PXE, CKD, diabetic |
| | | | | | calcification, atherocalcific disease |
| hMGP | Kozak | rENPP1 | FLAG | | GACI, calciphylaxis, PXE, CKD, diabetic |
| | | | | | calcification, atherocalcific disease, |
| | | | | | aortic valve calcification |

*optionally omitted in final constructs

In some embodiments, the constructs comprise a sequence provided herein, or are at least 80%, 85%, 90%, 95%, 97%, 98%, or 99% identical to a construct sequence set forth herein, optionally omitting any FLAG or other tag sequences, or any plasmid sequences, included in the sequences herein.

### Promoters

The constructs can include a promoter that drives expression of the ENPP1 sequence. As shown herein, the CMV promoter was able to drive expression of srENPP1 and rENPP1 proteins that showed catalytic activity in an assay.

In some embodiments, the promoter is a vascular endothelial cell-specific promoter, e.g., VE-cadherin promoter, fins-like tyrosine kinase-1 (FLT-1), intercellular adhesion molecule-2 (JCAM-2), a Claudin 5 (CLDN-5), a von Willebrand factor (vWF) promoter, a TIE2 promoter, or a synthetic EC-specific promoter (see, e.g., Dai et al., J Virol. 2004 June; 78(12): 6209-6221) or SMC-specific promoter as described herein, e.g., HDAC9 or SM22 or MYH11 or MGP, or promoters from albumin or hepcidin, ENPP1, ENPP2, and ENPP3. In some embodiments, the promoter is a pan-cell type promoter, e.g., a “ubiquitous” promoter that drives expression in most cell types, e.g., cytomegalovirus (CMV) promoter (optionally with the CMV enhancer), chicken beta-actin (CBA) promoter, Rous sarcoma virus (RSV) LTR promoter (optionally with the RSV enhancer), SV40 promoter, dihydrofolate reductase promoter, phosphoglycerol kinase promoter, phosphoglycerol kinase (PGK) promoter, EF1alpha promoter, Ubiquitin C (UBC), B-glucuronidase (GUSB), and CMV immediate/early gene enhancer/CBA promoter (CAG); or a steroid promoter or metallothionein promoter. Other promoters may be used, including smooth-muscle specific promoters from HDAC9 or SM22 or MYH11 or MGP, or promoters from albumin or hepcidin, ENPP1, ENPP2, and ENPP3. Exemplary sequences for these promoters are provided herein.

### Linkers

The constructs can also include linkers between the ENPP1 proteins and stabilizing proteins, which can include any sequence that does not interfere with the function of the ENPP1 proteins. In preferred embodiments, the linkers are short, e.g., 2-40 amino acids, and are typically flexible (i.e., comprising amino acids with a high degree of freedom such as glycine, alanine, and serine). In some embodiments, the linker comprises one or more units consisting of GGGS (SEQ ID NO:5) or GGGGS (SEQ ID NO:6), e.g., two, three, four, or more repeats of the GGGS (SEQ ID NO:5) or GGGGS (SEQ ID NO:6) unit. In some embodiments, the linker comprises an XTEN linker (e.g., a 32 amino acid modified XTEN linker (flanked with extended GlySer linkers on both sides)). Other linker sequences can also be used.

### Kozak Sequence

The constructs typically include a Kozak sequence at the 5′ end of the construct; a consensus Kozak sequence is generally considered as GCCGCCACCATGG (SEQ ID NO:7), where ATG is the start codon.

### Secretory Signal Sequence

A number of secretory signal peptide sequences are known in the art, including human signal sequences, examples of which are shown in Table A (Table adapted from novoprolabs.com/support/articles/commonly-used-leader-peptide-sequences-forefficient-secretion-of-a-recombinant-protein-expressed-in-mammalian-cells-201804211337.html).

TABLE A
Exemplary Human Secretory Signal Peptide Sequences

| Human Signal sequence | Sequence | SEQ ID NO: |
|---|---|---|
| Oncostatin M | MGVLLTQRTLLSLVLALLFPSMASM | 8. |
| IgG2 H | MGWSCIILFLVATATGVHS | 9. |
| Secrecon* | MWWRLWWLLLLLLLLWPMVWA | 10. |
| IgK VIII | MDMRVPAQLLGLLLLWLRGARC | 11. |
| CD33 | MPLLLLLPLLWAGALA | 12. |
| tPA | MDAMKRGLCCVLLLCGAVFVSPS | 13. |
| Chymotrypsinogen | MAFLWLLSCWALLGTTFG | 14. |
| trypsinogen-2 | MNLLLILTFVAAAVA | 15. |
| Interleukin 2 (IL-2) | MYRMQLLSCIALSLALVINS | 16. |
| Albumin (HSA) | MKWVTFISLLFSSAYS | 17. |
| Transthyretin | MASHRLLLLCLAGLVFVSEA | 18. |
| ENPP2 | MARRSSFQSCQIISLFTFAVGVNICLG | 19. |
| insulin | MALWMRLLPLLALLALWGPDPAAA | 20. |
| alpha 1-antitrypsin | MPSSVSWGILLLAGLCCLVPVSLA | 21. |

*, Barash et al., Biochem Biophys Res Commun. 2002 Jun. 21;294(4): 835-42.

In some embodiments, another signal sequence that promotes secretion is used, e.g., as described in Table 5 of U.S. Ser. No. 10/993,967, von Heijne, J Mol Biol. 1985 Jul. 5; 184(1):99-105; Kober et al., Biotechnol. Bioeng. 2013; 110: 1164-1173; Tsuchiya et al., Nucleic Acids Research Supplement No. 3 261-262 (2003). In some embodiments, the signal sequence is not an azuricidin signal sequence.

### Bone Targeting Peptide Sequences

The present constructs can also include one or more bone targeting sequences, e.g.,

(SEQ ID NO: 22)
AAGAATTTCCAGAGCAGAAGCCAC;
(SEQ ID NO: 23)
AAGAGAAGAACCCCTGTGCGGGAG;
(SEQ ID NO: 24)
AAGACCTACGCCTCTATGCAGTGG;
or
(SEQ ID NO: 25)
GATGATGACGACGACGATGACTGC.

See, e.g., Bang et al., Sci Rep. 2020; 10: 10576; Kim et al., Adv Sci (Weinh). 2023 October; 10(28): 2301570. In some embodiments, the bone targeting peptide is not Asp₁₀, or does not contain a consecutive stretch of 4 or more acidic amino acids, for example, glutamic acids or aspartic acids.

In some embodiments, the construct does not include an azuricidin peptide.

microRNA Target Sequences

microRNA (miRNA)-dependent post-transcriptional suppression of transgene expression can be used to increase specificity of vector-mediated transgene expression. MicroRNAs typically regulate gene expression by binding to sequences in the 3′ untranslated region (UTR) of the mRNA. To control exogenous transgene expression, tandem repeats (e.g., 1, 2, 3, 4, or 5, but preferably 3 repeats) of artificial microRNA target sites (also referred to as targets) can be incorporated into the 3′ UTR of the ENPP1 transgene expression cassette, leading to subsequent degradation of transgene mRNA in cells expressing the corresponding microRNA, thereby decreasing expression, e.g., as shown in FIG. 16 as well as FIG. 14B and FIG. 15. See, e.g., Geisler and Fechner, World J Exp Med. 2016 May 20; 6(2):37-54. Artificial miR-target for miRs useful in the present constructs can include those that have altered expression in T2D/CKD/CAD (21-3p, 155-5p, 126-5p) or are conserved for binding to the endogenous ENPP1 3′UTR (873-5p, 653-5p, 151-3p). In some embodiments, miRNA 155, miR21, miR122, miR210, miR30b, miR103, and/or miR82 binding sites can be incorporated into the 3′ UTR. In some embodiments, the miR target is for miR155, as it is decreased in coronary artery disease (CAD) and chronic kidney disease (CKD) but not in type 2 diabetes (T2D).

Target Sequences

| Micro | | | |
|---|---|---|---|
| RNA | Sequence | Target sequence | NCBI ref |
| miR155 | TTAATGCTAATCGTGATAGGGGTT | AATTACGATTAGCACTATCCCCAA | NR_ |
| | (SEQ ID NO: 26) | (SEQ ID NO: 27) | 030784.1 |
| miR-21 | TAGCTTATCAGACTGATGTTGA | ATCGAATAGTCTGACTACAACT | NR_ |
| | (SEQ ID NO: 28) | (SEQ ID NO: 29) | 029493.1 |
| miR- | AGCCCCTGCCCACCGCACACTG | TCGGGGACGGGTGGCGTGTGAC | NR_ |
| 210 | (SEQ ID NO: 30) | (SEQ ID NO: 31) | 029623.1 |
| miR- | TGTAAACATCCTACACTCAGCT | ACATTTGTAGGATGTGAGTCGA | NR_ |
| 30B | (SEQ ID NO: 32) | (SEQ ID NO: 33) | 029666.1 |
| miR- | TCATAGCCCTGTACAATGCTGCT | AGTATCGGGACATGTTACGACGA | NR_ |
| 103B1 | (SEQ ID NO: 34) | (SEQ ID NO: 35) | 031721.1 |
| miR- | GCAGGAACTTGTGAGTCTCCT (SEQ | CGTCCTTGAACACTCAGAGGA | NR_ |
| 873-5p | ID NO: 36) | (SEQ ID NO: 37) | 030618.1 |
| miR- | GTGTTGAAACAATCTCTACTG (SEQ | CACAACTTTGTTAGAGATGAC | NR_ |
| 653-5P | ID NO: 38) | (SEQ ID NO: 39) | 030388.1 |
| miR- | TCGAGGAGCTCACAGTCT (SEQ ID | AGCTCCTCGAGTGTCAGA | NR_ |
| 151b | NO: 40) | (SEQ ID NO: 41) | 039601.1 |
| miR- | TGTTTGTGGTAACAGTGTGAGGT | ACAAACACCATTGTCACACTCCA | NR_ |
| 122 | (SEQ ID NO: 105) | (SEQ ID NO: 106) | 029667.1 |
| miR- | CATTATTACTTTTGGTACGCG (SEQ | GTAATAATGAAAACCATGCGC | NR_ |
| 126-5p | ID NO: 42) | (SEQ ID NO: 43) | 029695.1 |

### Stabilizing Protein

The constructs can preferably include a stabilizing protein that is linked to and increases the half-life of the ENPP1 protein. Exemplary stabilizing proteins include human albumin, transthyretin, transferrin, and IgG Fc.

### Delivery Methods

The present methods and compositions can include the delivery of the gene therapy constructs described herein, e.g., via viral vectors or nanoparticles.

### Viral Vectors

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid. Viral vectors for use in the present methods and compositions include recombinant retroviruses, adenovirus, adeno-associated virus, alphavirus, and lentivirus, comprising the targeting peptides described herein and optionally a transgene for expression in a target tissue.

A preferred viral vector system useful for delivery of nucleic acids in the present methods is the adeno-associated virus (AAV). AAV is a tiny non-enveloped virus having a 25 nm capsid. No disease is known or has been shown to be associated with the wild-type virus. AAV has a single-stranded DNA (ssDNA) genome. AAV has been shown to exhibit long-term episomal transgene expression. Space for exogenous DNA in AAV is generally limited to an amount of nucleic acid that can physically fit inside the particle. For example, AAV types 1-5 can package up to 6 kb DNA, and in some reports AAV5 has been shown to package up to 8.9 kb DNA. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). There are numerous alternative AAV variants (over 100 have been cloned), and AAV variants have been identified based on desirable characteristics. The present disclosure contemplates uses of peptides that can be incorporated into an AAV capsid-thus providing capsid modified AAVs, e.g., AAVPR for selectively transfecting endothelium, pericytes and SMC after delivery to a subject. Such AAV's can also be used for delivery of a nucleic acid comprising an HDAC9-derived promoter as described herein. In some embodiments, an AAV suitable for use with a nucleic acid or a targeting peptide of the disclosure is AAV1, AAV2, AAV3, AAV4, AAV5, AAV6, AV6.2, AAV7, AAV8, rh.8, AAV9, rh.10, rh.39, rh.43 or CSp3; for CNS use, in some embodiments the AAV is AAV1, AAV2, AAV4, AAV5, AAV6, AAV8, or AAV9.

In some embodiments, the present methods use vessel-specific viral vectors that have been shown to transduce cerebral vasculature in large mammals and humans, including AAV such as AAV2 or 9, as well as capsid-modified AAVs that have improved specificity, transient expression, and/or higher transduction efficiency for SMCs including AAV9-PR, a modified version of AAV9 described herein and in WO2022232327 (which is incorporated by reference herein in its entirety). AAVPR comprises the sequence PRPPSTH (SEQ ID NO:44) in the capsid (i.e., inserted into the VP1 protein in a position corresponding to amino acids 588 and 589, see WO2022232327; alternatively, AAV-MA (comprising the sequence MAEPGAR (SEQ ID NO:45)), AAV-ML (comprising the sequence MLYADNT (SEQ ID NO:46), or AAV-SQ (comprising the sequence SQDPSTL (SEQ ID NO:47) inserted into the VP1 protein in a position corresponding to amino acids 588 and 589. AAVPR has been shown to have highly efficient transduction of endothelium and pericytes. AAVPR transduced the intima of capillaries, perforating arterioles and subarachnoid cerebral arteries (GFP) and vascular smooth muscle cells of cerebral arteries (SMCs). See Ramirez et al., Hum Gene Ther. 2023 August; 34(15-16):682-696. In cases where an AAV vector is used, the gene therapy construct can also include components such as inverted terminal repeats (ITRs) and Rep.cl

### Nanoparticles

In some embodiments, the ENPP1 polynucleotides as disclosed herein for delivery to a target tissue in vivo are encapsulated or associated with in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171.1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707.2005); and Zimmermann E et al, Electrolyte- and pH-stabilities of aqueous solid lipid nanoparticle (SLN) dispersions in artificial gastrointestinal media. Eur J Pharm Biopharm 52:203. 2001). In some embodiments, one or more polynucleotides is delivered to a target tissue in vivo in a vesicle, e.g., a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid). In some embodiments, lipid-based nanoparticles (LNP) are used; see, e.g., Robinson et al., Mol Ther. 2018 Aug. 1; 26(8):2034-2046; U.S. Pat. No. 9,956,271B2.

The present methods and compositions can include microvesicles or a preparation thereof that contains one or more therapeutic molecules, e.g., polynucleotides or RNA, as described herein. “Microvesicles”, as the term is used herein, refers to membrane-derived microvesicles, which includes a range of extracellular vesicles, including exosomes, microparticles and shed microvesicles secreted by many cell types under both normal physiological and pathological conditions. See, e.g., EP2010663B1. The methods and compositions described herein can be applied to microvesicles of all sizes. In some embodiments, 30 to 200 nm. In some embodiments, 30 to 800 nm. In some embodiments, up to 2 μm. The methods and compositions described herein can also be more broadly applied to all extracellular vesicles, a term which encompasses exosomes, shed microvesicles, oncosomes, ectosomes, and retroviral-like particles. Such a microvesicle or preparation is produced by the herein described methods. As the term is used herein, a microvesicle preparation refers to a population of microvesicles obtained/prepared from the same cellular source. Such a preparation is generated, for example, in vitro, by culturing cells expressing the nucleic acid molecule of the instant invention and isolating microvesicles produced by the cells. Methods of isolating such microvesicles are known in the art (Thery et al., Isolation and characterization of exosomes from cell culture supernatants and biological fluids, in Current Protocols Cell Biology, Chapter 3, 322, (John Wiley, 2006); Palmisano et al., (Mol Cell Proteomics. 2012 August; 11(8):230-43) and Waldenstrom et al., ((2012) PLoS ONE 7(4): e34653)), some examples of which are described herein. Such techniques for isolating microvesicles from cells in culture include, without limitation, sucrose gradient purification/separation and differential centrifugation, and can be adapted for use in a method or composition described herein. See, e.g., EP2010663B1.

In some embodiments, the microvesicles are isolated by gentle centrifugation (e.g., at about 300 g) of the culture medium of the donor cells for a period of time adequate to separate cells from the medium (e.g., about 15 minutes). This leaves the microvesicles in the supernatant, to thereby yield the microvesicle preparation. In some embodiments, the culture medium or the supernatant from the gentle centrifugation, is more strongly centrifuged (e.g., at about 16,000 g) for a period of time adequate to precipitate cellular debris (e.g., about 30 minutes). This leaves the microvesicles in the supernatant, to thereby yield the microvesicle preparation. In some embodiments, the culture medium, the gentle centrifuged preparation, or the strongly centrifuged preparation is subjected to filtration (e.g., through a 0.22 um filter or a 0.8 um filter, whereby the microvesicles pass through the filter. In some embodiments, the filtrate is subjected to a final ultracentrifugation (e.g., at about 110,000 g) for a period of time that will adequately precipitate the microvesicles (e.g. for about 80 minutes). The resulting pellet contains the microvesicles and can be resuspended in a volume of buffer that yields a useful concentration for further use, to thereby yield the microvesicle preparation. In some embodiments, the microvesicle preparation is produced by sucrose density gradient purification. In some embodiments, the microvesicles are further treated with DNAse (e.g., DNAse I) and/or RNAse and/or proteinase to eliminate any contaminating DNA, RNA, or protein, respectively, from the exterior. In some embodiments, the microvesicle preparation contains one or more RNAse inhibitors.

The molecules contained within the microvesicle preparation will comprise the therapeutic molecule. Typically the microvesicles in a preparation will be a heterogeneous population, and each microvesicle will contain a complement of molecule that may or may not differ from that of other microvesicles in the preparation. The content of the therapeutic molecules in a microvesicle preparation can be expressed either quantitatively or qualitatively. One such method is to express the content as the percentage of total molecules within the microvesicle preparation. By way of example, if the therapeutic molecule is an mRNA, the content can be expressed as the percentage of total RNA content, or alternatively as the percentage of total mRNA content, of the microvesicle preparation. Similarly, if the therapeutic molecule is a protein, the content can be expressed as the percentage of total protein within the microvesicles. In some embodiments, therapeutic microvesicles, or a preparation thereof, produced by the method described herein contain a detectable, statistically significantly increased amount of the therapeutic molecule as compared to microvesicles obtained from control cells (cells obtained from the same source which have not undergone scientific manipulation to increase expression of the therapeutic molecule). In some embodiments, the therapeutic molecule is present in an amount that is at least about 10%, 20%, 30% 40%, 50%, 60%, 70% 80% or 90%, more than in microvesicles obtained from control cells. Higher levels of enrichment may also be achieved. In some embodiments, the therapeutic molecule is present in the microvesicle or preparation thereof, at least 2 fold more than control cell microvesicles. Higher fold enrichment may also be obtained (e.g., 3, 4, 5, 6, 7, 8, 9 or 10 fold).

In some embodiments, a relatively high percentage of the microvesicle content is the therapeutic molecule (e.g., achieved through overexpression or specific targeting of the molecule to microvesicles). In some embodiments, the microvesicle content of the therapeutic molecule is at least about 10%, 20%, 30% 40%, 50%, 60%, 70% 80% or 90%, of the total (like) molecule content (e.g., the therapeutic molecule is an mRNA and is about 10% of the total mRNA content of the microvesicle). Higher levels of enrichment may also be achieved. In some embodiments, the therapeutic molecule is present in the microvesicle or preparation thereof, at least 2 fold more than all other such (like) molecules. Higher fold enrichment may also be obtained (e.g., 3, 4, 5, 6, 7, 8, 9 or 10 fold).

### Lipid Nanoparticles (LNPs)

The present methods and compositions can include the use of lipid nanoparticles (LNPs) for active delivery of target nucleic acids to smooth muscle cells (SMCs), in particular to vascular smooth muscle cells (vSMCs). Passive targeting of LNPs in the body is believed to be governed primarily by the size and charge of the LNP, which is acquired through changes in the molar compositions of the four types of lipids used in the formulation. In some aspects, the instant disclosure provides for lipid nanoparticles that comprise a permanently cationic lipid, in addition to having cholesterol, helper lipid(s), PEGylated lipid(s), and ionizable amine-containing lipid(s). The present disclosure demonstrates that certain ranges of permanently cationic lipids in formulations provide for particles and formulations with preferred tropism toward SMCs, in particular vSMCs. In some instances, such particles and formulations comprise from 0.1% to 85%, or 50% to 85%, or about 80% (molar percentage) of 1,2-Dioleoyl-3-trimethylammonium propane (often abbreviated DOTAP or 18:1TAP), a di-chain, or gemini, cationic surfactant molecule of formula I.

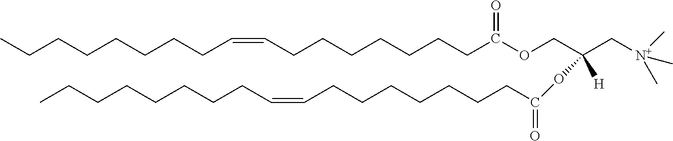 [SMILES: CCCCCCCC/C=C\CCCCCCCC(=O)OC[C@H](C[N+](C)(C)C)OC(=O)CCCCCCC/C=C\CCCCCCCC]

In some aspects, provided herein are lipid nanoparticles comprising certain amounts of DOTAP, an ionizable lipid, a neutral lipid, cholesterol, and one or more PEG-lipids, with demonstrable tropism towards smooth muscle cells. In some instances, the particles comprise an ionizable lipid, a neutral lipid, cholesterol, and one or more PEG-lipids at a molar ratio of about 50/10/38.5/1.5. In some instances, the particles comprise an ionizable lipid, a neutral lipid, cholesterol, and one or more PEG-lipids at a molar ratio of about 10/2.1/7.6/1.5. In many instances, the percentage of the ionizable lipid, the neutral lipid (e.g., phospholipid), the cholesterol, and the one or more PEG-lipids in a particle is selected to accommodate the incorporation of DOTAP into the particle. Specifically, in instances where the amounts of DOTAP in a particle are selected to range from 0.1% to 85% (molar percentage) (e.g., about 10% to about 80%, about 40% to about 85%, about 50% to about 85%, about 75% to about 85%, or about 0.1 to about 10%) of the total amounts of lipids in the particles. The amounts of the other lipids in the particle can be adjusted to conform to the amounts of DOTAP. For example, the amounts of ionizable lipid can be adjusted to range from 5%, 6%, 7%, 8%, 9%, 10%, 11%, 12%, 13%, 14%, 15%, 45%, 46%, 47%, 48%, 49%, 50%, 51%, up to 52% (molar percentage); amounts of a neutral lipid can be adjusted to range from 1%, 2%, 3%, 9%, 10%, 11% (molar percentage), amounts of cholesterol can be adjusted to range from 5%, 6%, 7%, 8%, 9%, 10%, 11%, 30%, 31%, 32%, 33%, 34%, 35%, 36%, 37%, 38%, 39%, up to 40% (molar percentage); and amounts of the one or more PEG-lipids can be adjusted from 0.1%, 1%, 1.5%, up to 2% (molar percentage) (e.g., about 0.1% to about 0.15%, about 0.1% to about 0.3%, about 0.1% to about 0.6%, about 0.1% to about 0.9%, about 0.1% to about 1.2%, about 0.1% to about 1.4%, about 0.1% to about 2%, about 0.15% to about 0.3%, about 0.15% to about 0.6%, about 0.15% to about 0.9%, about 0.15% to about 1.2%, about 0.15% to about 1.4%, about 0.15% to about 2%, about 0.3% to about 0.6%, about 0.3% to about 0.9%, about 0.3% to about 1.2%, about 0.3% to about 1.4%, about 0.3% to about 2%, about 0.6% to about 0.9%, about 0.6% to about 1.2%, about 0.6% to about 1.4%, about 0.6% to about 2%, about 0.9% to about 1.2%, about 0.9% to about 1.4%, about 0.9% to about 2%, about 1.2% to about 1.4%, about 1.2% to about 2%, about 0.10% to about 1.5%, about 0.2% to about 1.5%, about 0.3% to about 1.5%, about 0.4% to about 1.5%, about 0.5% to about 1.5%, about 0.6% to about 1.5%, about 0.7% to about 1.5%, about 0.8% to about 1.5%, about 0.9% to about 1.5%, about 1% to about 1.5%, about 1.1% to about 1.5%, about 1.2% to about 1.5%, about 1.3% to about 1.5%, about 1.4% to about 1.5%, 1.5% to about 1.6%, 1.5% to about 1.7%, 1.5% to about 1.8%, 1.5% to about 1.9%, or 1.5% to about 2%) of the total amounts of lipids (% of total lipids) in the particle.

In some embodiments, the LNPs include about 80% (molar percentage) of DOTAP. In some embodiments, the LNPs include about 78.8% of DOTAP. In some embodiments, the LNPs include about 75% to about 85% (e.g., about 75% to about 78.8%, about 75% to about 79%, about 75% to about 80%, about 75% to about 81%, about 75% to about 82%, about 75% to about 83%, about 75% to about 84%, about 75% to about 85%, about 76% to about 78.8%, about 76% to about 79%, about 76% to about 80%, about 76% to about 81%, about 76% to about 82%, about 76% to about 83%, about 76% to about 84%, about 76% to about 85%, about 77% to about 78.8%, about 77% to about 79%, about 77% to about 80%, about 77% to about 81%, about 77% to about 82%, about 77% to about 83%, about 77% to about 84%, about 77% to about 85%, about 78% to about 78.8%, about 78% to about 79%, about 78% to about 80%, about 78% to about 81%, about 78% to about 82%, about 78% to about 83%, about 78% to about 84%, about 78% to about 85%, about 79% to about 80%, about 79% to about 81%, about 79% to about 82%, about 79% to about 83%, about 79% to about 84%, about 79% to about 85%, about 80% to about 81%, about 80% to about 82%, about 80% to about 83%, about 80% to about 84%, or about 80% to about 85%) of DOTAP. In some embodiments, the LNPs include about 0.1% to about 10% DOTAP.

In some embodiments, the LNPs include about 10% of an ionizable lipid. In some embodiments, the LNPs include about 5% to about 15% (e.g., about 6% to about 10%, about 7% to about 10%, about 8% to about 10%, about 9% to about 10%, about 10% to about 11%, about 10% to about 12%, about 10% to about 13%, about 10% to about 14%, or about 10% to about 15%) of an ionizable lipid.

In some embodiments, the LNPs include about 2.1% of a neutral lipid. In some embodiments, the LNPs include about 2% of a neutral lipid. In some embodiments, the LNPs include about 0.5% to about 3.5% (e.g., about 0.5% to about 2.1%, about 0.6% to about 2.1%, about 0.7% to about 2.1%, about 0.8% to about 2.1%, about 0.9% to about 2.1%, about 1% to about 2.1%, about 1.1% to about 2.1%, about 1.2% to about 2.1%, about 1.3% to about 2.1%, about 1.4% to about 2.1%, about 1.5% to about 2.1%, about 1.6% to about 2.1%, about 1.7% to about 2.1%, about 1.8% to about 2.1%, about 1.9% to about 2.1%, about 2% to about 2.1%, about 2.1% to about 2.2%, about 2.1% to about 2.3%, about 2.1% to about 2.4%, about 2.1% to about 2.5%, about 2.1% to about 2.6%, about 2.1% to about 2.7%, about 2.1% to about 2.8%, about 2.1% to about 2.9%, about 2.1% to about 3.0%, about 2.1% to about 3.1%, about 2.1% to about 3.2%, about 2.1% to about 3.3%, about 2.1% to about 3.4%, or about 2.1% to about 3.5%) of a neutral lipid.

In some embodiments, the LNPs include about 7.6% of cholesterol. In some embodiments, the LNPs include about 7% to about 8% of a neutral lipid. In some embodiments, the LNPs include about 5% to about 10% (e.g., about 5% to about 7.6%, about 6% to about 7.6%, about 7% to about 7.6%, about 7% to about 8%, about 7% to about 9%, about 7% to about 10%, about 7.6% to about 8%, about 7.6% to about 9%, about 7.6% to about 10%) of a neutral lipid.

In some embodiments, the LNPs include one or more PEG-lipids comprising DMG-PEG and DSPE-PEG-maleimide (DSPE-PEG-mal). In some embodiments, the LNPs include about 1.2% DMG-PEG and about 0.3% DSPE-PEG-mal. In some embodiments, the LNPs include about 0.3% to about 1.2% of DMG-PEG and about 0% to about 0.6% DSPE-PEG-mal. In some embodiments, the LNPs include about 0% to about 1.5% (e.g., about 0% to about 0.3%, about 0.3% to about 0.75%, about 0.3% to about 1%, about 0.3% to about 1.05%, about 0.3% to about 1.2%, about 0.3% to about 1.5%) of DMG-PEG. In some embodiments, the LNPs include about 0% to about 1% (e.g., about 0% to about 0.3%, about 0% to about 0.4%, about 0% to about 0.5%, about 0% to about 0.6%, about 0.1% to about 0.3%, about 0.1% to about 0.4%, about 0.1% to about 0.5%, about 0.1% to about 0.6%, about 0.2% to about 0.3%, about 0.2% to about 0.4%, about 0.2% to about 0.5%, about 0.2% to about 0.6%, about 0.3% to about 0.4%, about 0.3% to about 0.5%, about 0.3% to about 0.6%, about 0.3% to about 1%, or about 0.6% to about 1%) of DSPE-PEG-mal.

Specifically, amounts of DOTAP in a particle of the disclosure can be specified in terms of total lipid percentage. Specifically, in instances where the percentage of DOTAP in a particle is selected to range from about 0.1% to about 80% of the total percentage of lipids in the particles, the amounts of the other lipids in the particle can be adjusted based on the remaining lipid percentage as follows: amounts of ionizable lipid can be adjusted to range from 10% up to 52% of the remaining lipid percentage; amounts of a neutral lipid can be adjusted to range from 2% up to 11% of the remaining lipid percentage, amounts of cholesterol can be adjusted to range from 7% up to 40% of the remaining lipid percentage; and amounts of one or more PEG-lipids can be adjusted from 0% up to 2% of the remaining lipid percentage.

For example, when up to 10% DOTAP is added, the remaining 90% of the total lipid amount is distributed accordingly among the remaining lipids. For example, in some embodiments, if the percentage of DOTAP in a particle is 10%, the amounts of the other lipids in the particle can be adjusted based on the remaining 90% as follows: amounts of ionizable lipid can be adjusted to range from 45% up to 52% of the remaining 90%; amounts of a neutral lipid can be adjusted to range from 9% up to 11% of the remaining 90%, amounts of cholesterol can be adjusted to range from 34% up to 40% of the remaining 90%; and amounts of a PEG-lipid can be adjusted from 0.1% up to 2% of the remaining 90% of the total lipids in the composition.

In another example, when up to 80% DOTAP is added, the remaining 20% of the total lipid amount is distributed accordingly among the remaining lipids. For example, in some embodiments, if the percentage of DOTAP in a particle is 80%, the amounts of the other lipids in the particle can be adjusted based on the remaining 10% as follows: amount of ionizable lipid can be adjusted to range from 7% up to 13% of the remaining 10%; amount of a neutral lipid can be adjusted to range from 1% up to 3% of the remaining 10%, amount of cholesterol can be adjusted to range from 6% up to 8% of the remaining 10%; and amounts of the one or more PEG-lipids can be adjusted from 0.1% up to 2% of the remaining 10% of the total lipids in the composition.

In some embodiments, the LNPs provided herein can be spherical or ellipsoidal, or can have an amorphous shape. In some embodiments, the LNPs provided herein (e.g., conjugated or non-conjugated LNPs) can have a diameter (between any two points on the exterior surface of the LNP) of between about 100 nanometers (nm) to about 250 nm (e.g., between about 100 nm to about 150 nm, between about 100 nm to about 200 nm, between about 100 nm to about 250 nm, between about 125 nm to about 150 nm, between about 150 nm to about 175 nm, between about 150 nm to about 200 nm, between about 150 nm to about 250 nm). In some embodiments, LNPs having a diameter of between about 100 nm to about 250 nm localize to the diseased vasculature in a subject. In some embodiments, LNPs having a diameter of between about 100 nm to about 150 nm localize to the smooth muscle cells of a subject.

### Lipid Nanoparticles (LNPs)

The LNP compositions can be prepared by various techniques which are presently known in the art. Multilamellar vesicles (MHLVs) may be prepared conventional techniques, for example, by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Unilamellar vesicles (ULVs), such as the LNPs of the disclosure, can then be formed by homogenization, sonication, or extrusion of the multi-lamellar vesicles. In addition, unilamellar vesicles can be formed by detergent removal techniques.

In many instances, the particles, formulations, and compositions of the disclosure comprise at least the following five lipid components:

### Permanently Cationic Lipids

Structurally, synthetic and/or natural lipids usually contain three parts: (i) cationic or ionizable head groups, (ii) linker groups, and (iii) hydrophobic tails. The chemical diversity of each part results in a number of structurally distinct ionizable lipids that can be produced by combinatorial chemistry. Conventional permanently charged cationic lipids previously used for nucleic acid delivery (e.g., DOTAP) are believed to readily interact with negatively charged serum proteins and aggregate in the bloodstream, which was believed to lead to rapid clearance of LNP by mononuclear phagocytes. Thus, the relatively high hemolytic activity of cationic lipids was believed to increase the risk of toxic side effects, such as hemoglobin release due to red cell membrane damage.

The disclosure demonstrates that the presence of certain ratios or amounts of permanently cationic lipids (e.g., DOTAP) in a particle can preferably target the particle to vascular smooth blood cells (vSMCs), in vitro and in vivo. In some embodiments, the particles of the disclosure include DOTAP. In some embodiments, the particles of the disclosure include DOTAP, 1,2-di-O-octadecenyl-3-trimethylammonium propane (chloride salt) (DOTMA), dimethyldioctadecylammonium (DDAB), 1,2-dimyristoyl-sn-glycero-3-ethylphosphocholine (EPC), or any combination thereof.

### Ionizable Cationic Lipids

Ionizable cationic lipids are traditional components in many existing LNP formulation(s). Their acid dissociation constants (pKa) determine the ionization and surface charge of the LNP, further affecting its stability and toxicity. To avoid these problems, ionizable cationic lipids with pKa values typically ranging from 6.0 to 7.0 have been developed and deployed, most notably in vaccine formulations. This ionizable lipid-based LNP (iLNP) ensures efficient encapsulation of nucleic acids under acidic conditions and reduces toxicity during recycling under physiological conditions. After entering endosomes/lysosomes (which have a pH below surface pKa), LNPs can be positively charged again to facilitate endosome escape and release mRNA into the cytoplasm. It has been reported that LNPs with pKa values of 6.2-6.5 and 6.6-6.9 favored hepatic delivery of siRNA in vivo and intramuscular administration of mRNA vaccines, respectively.

Depending on the number of amino heads, ionizable cationic lipids can be classified as either monoamino or polyamino lipids. Non-limiting examples of monoamino acid ionizable cationic lipids contemplated in particles of the disclosure include DLin-MC3-DMA (MC3), SM-102, and ALC-0315. Non-limiting examples of monoamino acid ionizable cationic lipids contemplated in particles of the disclosure include 3060ᵢ₁₀, cKK-E12, C12-200, 5A2-SC8, TT3, and FTT5. In many instances, the ionizable lipid is selected from the group consisting of DLin-MC2-DMA, DLin-MC3-DMA, DSDMA, DODMA, DLinDMA, DLenDMA, γ-DLenDMA, DLin-K-DMA, DLin-C2K-DMA, DLin-K-C3-DM A, DLin-K-C4-DMA, DLen-C2K-DMA, γ-DLen-C2K-DMA, or DLin-MP-DMA.

### PEG-Lipids

Although PEG-lipids generally constitute the smallest molar percentage of the lipid components in LNPs (typically about 0.5 mol % and up to about 2.0 mol %), they have several effects on the properties of lipid nanoparticles, including influencing particle size and zeta potential. A variety of PEG lipids are contemplated for use with the LNPs of the disclosure, including terminally modified PEG lipids. The LNPs can include one, two, or more different species of PEG lipids.

PEG lipids for use in the present disclosure can be, for example, maleimide terminally modified PEG lipids that can be conjugated with cell targeting peptides. PEG lipids for use with the instant LNPs can have the general structure —(CH₂CH₂O)n- or —(CH₂CH₂O)nCH₂CH₂. This general structure can further be modified with heterobifunctional maleimide linker. The disclosure contemplates that a variety of PEG molecules can be incorporated into its LNPs, including poly(ethylene glycol) (PEG) maleimide (e.g., PEG-2000 maleimide), polyalkylene glycols, polypropylene or polybutylene glycols, methoxy poly (ethylene glycol), or methoxy poly (ethylene glycol) propionic acid (mPEG-acid) where n can be from about 1 to about 400. An LNP comprising a thiol reactive motive conjugated to a PEG molecule (e.g., heterobifunctional maleimide PEG) can be decorated with various types of peptides that are displayed on the surface of the LNP molecule.

In some instances, a PEG molecule is linked to a thiol reactive group for further conjugation to a peptide. In some instances the PEG molecule is a maleimide conjugated PEG molecule. Reactive PEGs can be used for amine pegylation, thiol pegylation, or N-terminal pegylation. The amine in the N-terminus and/or the carboxyl group in the C-terminus can react with a targeting peptide.

In some instances, the PEG-lipid that is suitable for use in the particles of the disclosure is 1,2-dimyristoyl-rac-glycero-3-methoxypolyethylene glycol (DMG-PEG) and/or DSPE-PEG (1,2-distearoyl-sn-glycero-3-phosphoethanolamine-N-[maleimide(polyethylene glycol). In some embodiments, the PEG-lipid that is suitable for use in the particles of the disclosure is DMG-PEG 2000 and/or DSPE-PEG 2000 (1,2-distearoyl-sn-glycero-3-phosphoethanolamine-N-[maleimide(polyethylene glycol)-2000]. In some instances, the PEG molecule is methoxy poly (ethylene glycol) succinimidyl proprionate (mPEG-SPA). In some instances, a PEG molecule is a methoxy poly (ethylene glycol) propionic acid (mPEG-acid). In some cases, the polyethylene glycol molecule weighs from about 1,000 kilodaltons to about 5,000 kilodaltons. The covalent attachment of a targeting peptide to an LNP via a thiol reactive linkage can change the physicochemical characteristics of the LNP. Examples of physicochemical characteristics that can be altered by binding to a PEG include its zeta potential, its PDI, and the overall hydrodynamic size of the LNP.

Non-limiting examples of commercially available PEGs suitable for use in the particles of the disclosure include, but are not limited to those available from Nektar Therapeutics, San Carlos, CA, such as mPEG-NH₂ (Mw about 10 kDa, about 20 kDa), methoxy PEG Succinimidyl α-Methylbutanoate (SMB), SMB-PEG-SMB, methoxy PEG Succinimidyl Propionate (mPEG-SPA), Branched PEG N-Hydroxysuccinimide (mPEG2-NHS), mPEG-CM-HBA-NHS, NHS-HBA-CM-PEG-CM-HBA-NHS, mPEG-ButyrALD, ButyrALD-PEG-ButyrALD, Branched PEG ButyrALD (mPEG2-ButyrALD), Ortho-pyridylthioester (mPEG-OPTE), mPEG Maleimide (MAL), MAL-PEG-MAL, Branched PEG Maleimide (mPEG2-MAL), Forked Maleimide (mPEG-MAL2 and mPEG2-MAL2), mPEG-Ortho-pyridyldisulfide (mPEG-OPSS), OPSS-PEG-OPSS, mPEG-SH, SH-PEG-SH, Amine-PEG-Acid, Boc-PEG-NHS, Fmoc-PEG-NHS, MAL-PEG-NHS, Vinylsulfone-PEG-NHS, Acrylate-PEG-NHS Ester. In some embodiments, the LNPs comprise DMG-PEG and/or DSPE-PEG-maleimide.

Non-limiting examples of PEGs that can be used in amine pegylation include, for example, PEGs manufactured by Jenken Technology USA such as: Y-shape PEG NHS Esters, Y-shape PEG Carboxyl, Glucose PEG NHS Ester, Galactose PEG NHS Ester, Methoxy PEG Succinimidyl Carboxymethyl Ester, Methoxy PEG Carboxyl, Methoxy PEG Succinimidyl Butanoate, Methoxy PEG Succinimidyl Hexanoate, Methoxy PEG Hexanoic Acid, Methoxy PEG Succinimidyl Succinamide, Methoxy PEG Succinimidyl Glutaramide, Methoxy PEG Succinimidyl Carbonate, Methoxy PEG Nitrophenyl Carbonate, Methoxy PEG Succinimidyl Succinate, Methoxy PEG Succinimidyl Glutarate. Non-limiting examples of PEGs that can be used in thiol pegylation include Y-shape PEG Maleimide, Methoxy PEG Maleimide, Methoxy PEG Vinylsulfone, Methoxy PEG Thiol. Non-limiting examples of PEGs that can be used in N-terminal pegylation include, for example, PEGs manufactured by Jenken Technology USA such as: Y-shape PEG Aldehyde, Y-shape PEG Acetaldehyde, Y-shape PEG Propionaldehyde, Methoxy PEG Propionaldehyde.

In many instances, a targeting peptide can have a molecular weight that is small compared to the PEG molecule to which it is attached. The molecular weight of a PEG molecule used in an LNP of the disclosure can be, for example, no greater than 5 kilodaltons (kDa), no greater than 4.5 kilodaltons, no greater than 4 kilodaltons, no greater 3.5 than kilodaltons (kDa), no greater than 3 kilodaltons (kDa), no greater than 2.5 kilodaltons (kDa), no greater than 2 kilodaltons (kDa), no greater than 1.5 kilodaltons (kDa), or no greater than 1 kilodaltons (kDa).

In some cases, the molecular weight of a PEG molecule can be greater than 1 kilodalton (kDa), greater than 1.5 kilodaltons (kDa), greater than 2 kilodaltons (kDa), greater than 2.5 kilodaltons (kDa), greater than 3 kilodaltons (kDa), greater than 3.5 kilodaltons (kDa), greater than 4 kilodaltons (kDa), or greater than 4.5 kilodaltons (kDa).

In some cases the molecular weight of a PEG oligomer can be from about 1 kilodalton (kDa) to about 5 kilodaltons (kDa), from about 1 kilodalton (kDa) to about 2 kilodaltons (kDa), from about 1 kilodaltons (kDa) to about 3 kilodaltons (kDa), from about 1 kilodaltons (kDa) to about 4 kilodaltons (kDa), from about 1 kilodaltons (kDa) to about 5 kilodaltons (kDa), from about 1.5 kilodaltons (kDa) to about 2 kilodaltons (kDa), from about 1.5 kilodaltons (kDa) to about 3 kilodaltons (kDa), from about 1.5 kilodaltons (kDa) to about 3.5 kilodaltons (kDa), from about 1.5 kilodaltons (kDa) to about 4 kilodaltons (kDa), from about 1.5 kilodaltons (kDa) to about 4.5 kilodaltons (kDa), from about 1.5 kilodaltons (kDa) to about 5 kilodaltons (kDa), from about 2 kilodaltons (kDa) to about 3 kilodaltons (kDa), from about 2 kilodaltons (kDa) to about 3.5 kilodaltons (kDa), from about 2 kilodaltons (kDa) to about 4 kilodaltons (kDa), from about 2 kilodaltons (kDa) to about 4.5 kilodaltons (kDa), from about 2 kilodaltons (kDa) to about 5 kilodaltons (kDa).

In some embodiments, the molecular weight of a maleimide-terminally modified PEG lipid is about 2 kilodaltons (kDa). In some embodiments, the molecular weight of a PEG molecule is from about 1 kilodaltons (kDa) to about 5 kilodaltons (kDa).

### Neutral-Lipids Helper Lipids—Phospholipids

Phospholipids are neutral “helper” lipids that contribute to the formation of lipid nanoparticles and the escape of endosomes. In many instances, a particle of the disclosure comprises a neutral lipid that is a phosphatidylcholine lipid or a phosphatidylethanolamine lipid. The phosphatidylcholine lipid or the phosphatidylethanolamine lipid can be selected from the group comprising 1,2-dioleoyl-sn-glycero-3-phosphoethanolamine (DOPE), 1,2-dioleoyl-sn-glycero-3-phosphocholine (DOPC), 1,2-distearoyl-sn-glycero-3-phosphocholine (DSPC), 1,2-dipalmitoyl-sn-glycero-3-phosphocholine (DPPC), 1-palmitoyl-2-oleoyl-glycero-3-phosphocholine (POPC), and 1-stearoyl-2-oleoyl-sn-glycero-3-phosphocholine (SOPC).

### Cholesterol

The inclusion of cholesterol in nucleic acid-containing LNP formulations is based primarily on two major findings obtained with liposomal formulations of small molecule therapeutics: 1) cholesterol is an exchangeable molecule that can accumulate within liposomes during circulation, 2) cholesterol dramatically reduces the amount of surface-bound proteins and improves the circulating half-life.

### Particles Comprising Cell Targeting Peptides

In some aspects, the disclosure provides “decorated” with peptides on their exterior surface. In order to enhance the targeting of the nanoparticles to specific tissue or cells, the disclosure further conjugated the aforementioned particles with peptides that can target tissue or cell surface receptors. In some embodiments, the LNPs described herein can contain at least one type (e.g., two, three, or four) of targeting peptides covalently-linked to the LNP. Targeting peptides often contain an amino acid sequence that is recognized by a molecule present on the surface of a cell (e.g., a cell type present in a target tissue). For example, a targeting peptide comprising a collagen IV-targeting peptide specifically binds to collagen IV receptors in the extracellular matrix of diseased vasculature. Additional non-limiting targeting peptides that can be covalently-linked to any of the therapeutic nanoparticles described herein include: an interleukin 6 receptor (IL-6R)-targeting peptide, a CD63-targeting peptide, a contiguous sequence of amino acids (e.g., at least 10, 15, or 20) present within a contiguous sequence of CD63, a galectin-3 (GAL-3)-targeting peptide, amino acids (e.g., at least 10, 15, or 20) present within Col-4 KLWVLPK-GGG-C(SEQ ID NO:48), TL6-R C-GGG-LSLITRL (SEQ ID NO:49), CD63 CRHSQMTVTSRL-GGG (SEQ ID NO:50), and/or Gal-3 C-GGG-ANTPCGPYTHDCPVKR (SEQ ID NO:51). Additional examples of targeting peptides are known in the art.

In some embodiments, the targeting peptide is covalently conjugated to the LNP by using a maleimide-terminally modified PEG lipid. Such conjugation includes the reaction of maleimides with thiol groups of the peptides to form thioether bonds. In some embodiments, peptide conjugation can be done in different densities controlled by the percentage of the maleimide-terminally modified PEG lipid in the LNP formulation (see, e.g., Example 4). In some embodiments, the amount of maleimide-terminally modified PEG lipid in the LNP formulation ranges from about 0.15% to about 1.2%. In some embodiments, the number of targeting peptide molecules that can decorate an outer surface of the particles of the disclosure ranges from about 192 targeting peptides per LNP to about 1270 targeting peptides per LNP (e.g., from about 192 to about 420 targeting peptides per LNP, from about 192 to about 609 targeting peptides per LNP, from about 192 to about 901 targeting peptides per LNP, from about 192 to about 1270 targeting peptides per LNP, from about 420 to about 609 targeting peptides per LNP, from about 420 to about 901 targeting peptides per LNP, from about 420 to about 1270 targeting peptides per LNP, from about 609 to about 901 targeting peptides per LNP, from about 609 to about 1270 targeting peptides per LNP, from about 901 to about 1270 targeting peptides per LNP).

In some embodiments, the targeting peptide can be covalently linked to the LNP at its N-terminus or at its C-terminus. In some embodiments, the targeting peptide can be covalently linked to the LNP through an amino acid side chain. Targeting peptides can be covalently-linked to any of the LNPs described herein through a chemical moiety containing a disulfide bond, an amide bond, or a thioether bond. Additional chemical moieties that can be used to covalently link a targeting peptide to a therapeutic nanoparticle are known in the art.

A variety of different methods can be used to covalently link a targeting peptide to a therapeutic nanoparticle. In some embodiments, the LNPs can be activated for attachment with a targeting peptide, for example in non-limiting embodiments, the LNPs can be epoxy-activated, carboxyl-activated, iodoacetyl-activated, aldehyde-terminated, amine-terminated, or thiol-activated. Additional methods for covalently linking a targeting peptide to a therapeutic nanoparticle are known in the art.

Preferably, the particles are conjugated with peptides (e.g., collagen IV peptides) that target receptors highly expressed in diseased vasculature extracellular matrix (collagen IV) in order to increase the accumulation and the retention of the nanoparticles in diseased tissues. For example, the particles can be conjugated with peptides that target receptors highly expressed on the surface of vascular SMCs (e.g., IL-6R, CD63, and/or GAL-3), thereby increasing the uptake into these cells. In some aspects, the disclosure further comprises particles comprising encapsulating a nucleic acid therapeutic cargo encoding a gene (e.g., ENPP1) for rescuing gene expression in a smooth muscle cell. The disclosure demonstrates that a combination of any of these three approaches can potentially increase accumulation and retention at the SMC target site and cell specificity.

In some aspects, the disclosure comprises a particle comprising: a) a molecule of formula I

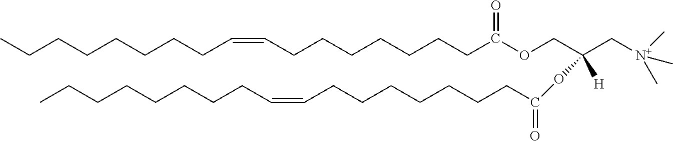 [SMILES: CCCCCCCC/C=C\CCCCCCCC(=O)OC[C@H](C[N+](C)(C)C)OC(=O)CCCCCCC/C=C\CCCCCCCC]

(DOTAP) (e.g., from 0.1% to 85% DOTAP); and a peptide conjugated to a linker molecule in the particle, wherein the peptide has an affinity for a vasculature extracellular matrix molecule. The particles can also comprise: a) from 0.1% to 85% of a molecule of formula I

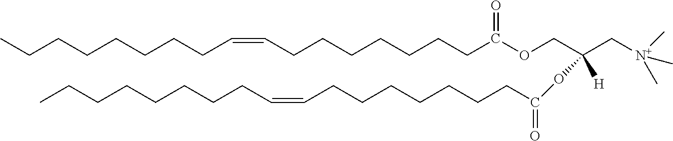 [SMILES: CCCCCCCC/C=C\CCCCCCCC(=O)OC[C@H](C[N+](C)(C)C)OC(=O)CCCCCCC/C=C\CCCCCCCC]

(DOTAP); and a nucleic acid therapeutic cargo encoding a gene for rescuing gene expression in a smooth muscle cell. In many instances, the composition also comprises an amount of an ionizable lipid; an amount of neutral lipid; an amount of cholesterol; and an amount of a PEG-lipid. In many instances, the concentration of DOTAP ranges from 0.1% to 85%, and the molar ratios of the ionizable lipid, the neutral lipid, the cholesterol, and the PEG-lipid are adjusted to a molar ratio of approximately 50/10/38.5/1.5. In some instances, the particles comprise an ionizable lipid, a neutral lipid, cholesterol, and one or more PEG-lipids at a molar ratio of about 10/2.1/7.6/1.5. In some embodiments, the amount of the ionizable lipid ranges from about 45% to about 52% or about 5% to about 15%. In some embodiments, the amount of the neutral lipid ranges from about 1% to about 3% or 9% to about 11%. In some embodiments, the amount of cholesterol ranges from about 5% to about 9% or about 34% to about 40%. In some embodiments, the amount of the one or more PEG-lipids range from about 0.1% to about 2%.

In some aspects, the linker molecule is a molecule that is used to covalently link the peptide to the LNP. In some embodiments, the linker molecule is a maleimide group at a PEG lipid in the particle as discussed supra. The peptide can be a collagen IV (Col-IV) peptide, an TL-6R peptide, a CD63, a GAL-3, and/or a functional fragment thereof sufficient for increasing an accumulation and the retention of the nanoparticles in target tissues. The neutral lipid can be a phosphatidylcholine lipid or a phosphatidylethanolamine lipid, such as the ones selected from the group consisting of DOPE, DOPC, DSPC, DPPC, POPC, and SOPC. The ionizable lipid can be selected from the group consisting of DLin-MC2-DMA, DLin-MC3-DMA, DSDMA, DODMA, DLinDMA, DLenDMA, γ-DLenDMA, DLin-K-DMA, DLin-C2K-DMA, DLin-K-C3-DM A, DLin-K-C4-DMA, DLen-C2K-DMA, γ-DLen-C2K-DMA, or DLin-MP-DMA.

Such particles can encapsulate any one of the aforementioned nucleic acid therapeutic cargo(s). In some embodiments, the therapeutic cargo is an mRNA molecule encoding a gene for rescuing gene expression in the smooth muscle cell. In some embodiments, the therapeutic cargo is a plasmid encoding a gene for rescuing gene expression in the smooth muscle cell. In some embodiments, the therapeutic cargo is a nucleic acid molecule encoding an ENPP1 therapeutic cargo. In some embodiments, the ENPP1 therapeutic cargo encodes a transmembrane ENPP1 molecule. In some embodiments, the ENPP1 therapeutic cargo encodes a soluble ENPP1 molecule. In some embodiments, the transmembrane ENPP1 molecule is or comprises a sequence at least 80% identical to SEQ ID NO: 1.

Further, provided are therapeutic formulations comprising: from 75% to 85% of a molecule of formula I

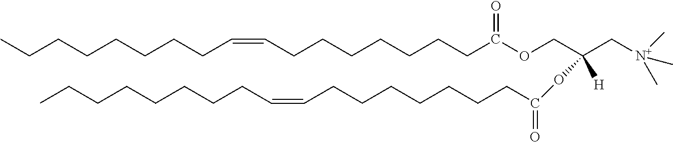 [SMILES: CCCCCCCC/C=C\CCCCCCCC(=O)OC[C@H](C[N+](C)(C)C)OC(=O)CCCCCCC/C=C\CCCCCCCC]

(DOTAP); an amount of an ionizable lipid; an amount of neutral lipid; an amount of cholesterol; an amount of a PEG-lipid; comprising amounts of the ionizable lipid, the neutral lipid, the cholesterol, and the PEG-lipid at a molar ratio of 50/10/38.5/1.5 or at a molar ratio of about 10/2.1/7.6/1.5; and a nucleic acid construct encoding an ectonucleotide pyrophosphatase/phosphodiesterase 1 (ENPP1) therapeutic cargo as described herein. The ENPP1 therapeutic cargo can be a transmembrane ENPP1 molecule or a soluble ENPP1 molecule. In some instances, the disclosure provides a therapeutic formulation comprising: about 10% of DOTAP; about 6% of an MC3 ionizable lipid; about 9.8% of a DOPE neutral lipid; about 35.3% of cholesterol; about 1.4% of one or more PEG-lipids; and a nucleic acid construct encoding an ectonucleotide pyrophosphatase/phosphodiesterase 1 (ENPP1) therapeutic cargo. In some instances, the disclosure provides a therapeutic formulation comprising: about 80% of DOTAP; about 10% of an MC3 ionizable lipid; about 2.1% of a DOPE neutral lipid; about 7.6% of cholesterol; about 1.5% of one or more PEG-lipids; and a nucleic acid construct encoding an ectonucleotide pyrophosphatase/phosphodiesterase 1 (ENPP1) therapeutic cargo. Exemplary formulations can comprise: about 75-85% of DOTAP; about 10% of an MC3 ionizable lipid; about 2-2.5% of a DOPE neutral lipid; about 7-8% of cholesterol; and about 1-2% of one or more PEG-lipids. For example, a preferred formulation can comprise about 78.8% of DOTAP; about 10% of an MC3 ionizable lipid; about 2.1% of a DOPE neutral lipid; about 7.6% of cholesterol; and about 1.5% of one or more PEG-lipids.

In some aspects, the disclosure comprises a therapeutic formulation comprising: a) from 75% to 85% of a molecule of formula I

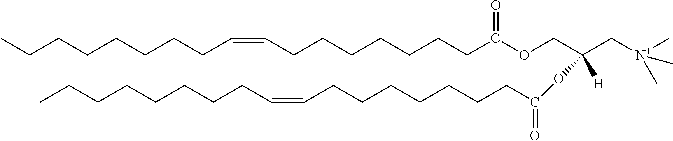 [SMILES: CCCCCCCC/C=C\CCCCCCCC(=O)OC[C@H](C[N+](C)(C)C)OC(=O)CCCCCCC/C=C\CCCCCCCC]

(DOTAP); and an amount of an ionizable lipid; an amount of neutral lipid; an amount of cholesterol; an amount of a PEG-lipid; comprising amounts of the ionizable lipid, the neutral lipid, the cholesterol, and the PEG-lipid at a molar ratio of 10/2.1/7.6/1.5; and a nucleic acid construct encoding a smooth muscle alpha (α)-2 actin (ACTA2) therapeutic cargo.

Exemplary formulations can comprise: about 75-85% of DOTAP; about 10% of an MC3 ionizable lipid; about 2-2.5% of a DOPE neutral lipid; about 7-8% of cholesterol; and about 1-2% of one or more PEG-lipids. For example, a preferred formulation can comprise about 78.8% of DOTAP; about 10% of an MC3 ionizable lipid; about 2.1% of a DOPE neutral lipid; about 7.6% of cholesterol; and about 1.5% of one or more PEG-lipids.

### Methods of Treatment

Provided herein are methods of treating a subject who has a condition associated with vascular calcification. Diseases that can be treated using the methods and compositions described herein include GACI, pseudoxanthoma elasticum (PXE), calciphylaxis, in-stent restenosis (which is due to proliferation of SMCs), graft stenosis (e.g., coronary artery bypass graft stenosis), and cardiovascular disease including diabetic vascular calcification, ESRD-associated vascular disease, calcific aortic valve disease (CAVD), coronary atherosclerosis, peripheral vascular disease, and cerebral atherosclerosis. In general, the methods include administering a therapeutically effective amount of an ENPP1 gene construct, e.g., using a viral vector such as an adeno-associated virus (AAV) or a lipid nanoparticle (LNP) carrying the ENPP1 gene construct, optionally including a CBA, CMV, CAG, or other promoter. The ENPP1 gene constructs are preferably administered intravenously, but can also be administered by other routes such as subcutaneous injection. Exemplary constructs are described herein and include those in Table B, optionally with additional components such as bone targeting peptide sequences.

In clinical settings, viral- or LNP-based ENPP1 gene therapy constructs can be introduced into a subject by any of a number of methods, each of which is familiar in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., PNAS USA 91: 3054-3057 (1994)).

A pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells, which produce the gene delivery system.

An “effective amount” is an amount sufficient to effect beneficial or desired results. For example, a therapeutic amount is one that achieves a desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic constructs can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Constructs that exhibit high therapeutic indices are preferred. While constructs that exhibit toxic side effects may be used, care should be taken to design a delivery system that targets such constructs to the site of affected tissue in order to minimize potential damage to unaffected cells and, thereby, reduce side effects.

Data obtained from cell culture assays and animal studies can be used in formulating a range of dosage for use in humans. The dosage of such constructs lies preferably within a range of circulating concentrations that include the ED50 with little or no toxicity. The dosage may vary within this range depending upon the dosage form employed and the route of administration utilized. For any construct used in a method described herein, a therapeutically effective dose can be estimated initially from animal models or based on other constructs. Such information can be used to more accurately determine useful doses in humans.

### Conditions Treatable Using the Present Methods and Compositions Include the Following:

Generalized arterial calcification of infancy (GACI): GACI is characterized by widespread arterial calcification and/or stenoses of large and medium-sized vessels resulting in a range of clinical manifestations including myocardial infarction, respiratory distress, hypertension, cardiomegaly, and stroke. GACI is estimated to affect one in 200,000 pregnancies. Mortality is particularly high in early infancy; approximately 55% of patients die within the first 6 months of life despite intensive care and supportive measures. After 6 months of life, the mortality rate is markedly reduced and patients tend to survive, though many still have sequalae from their initial hypoxic insults, and a majority eventually develop hearing loss and hypophosphatemic rickets. GACI typically results from biallelic loss-of-function mutations in ENPP1, which encodes an ectonucleotide pyrophosphatase/phosphodiesterase that converts ATP into AMP and pyrophosphate (PPi), a potent inhibitor of calcification. Loss of ENPP1 activity results in decreased quantity of PPi both locally and systemically, and GACI patients have low plasma and urinary PPi concentrations. A treatment as described herein can result in reduced arterial calcification and/or stenoses of large and medium-sized vessels, and increased plasma and/or urinary PPi concentrations (approaching, near, or within normal; normal plasma PPi is about 2.4-4.4 uM in humans, see Bernhard et al., J Clin Endocrinol Metab. 2022 January; 107(1): 109-118).

Pseudoxanthoma elasticum (PXE): PXE is caused by defects in the presumptive ATP-dependent exporter ABCC6, disrupts extracellular ATP metabolism resulting in calcification of elastic fibers in the skin, eyes, and arterial wall. Furthermore, mutations in ENPP1 have been described in patients with PXE. Null mice (Abcc6−/−) recapitulate the genetic, histopathologic and ultrastructural features of PXE, and they demonstrate early and progressive mineralization of vibrissae dermal sheath, which serves as a biomarker of the overall mineralization process. A treatment as described herein can result in reduced calcification of elastic fibers in the skin, eyes, and arterial wall, and/or reduced mineralization of vibrissae dermal sheath.

Calciphylaxis: Calciphylaxis is a rare, life-threatening disease of rapidly progressive vascular calcification characterized by microvascular occlusion in the dermis and subcutaneous tissue. Patients with calciphylaxis have limited survival of typically less than one year. They also have significant morbidity from cutaneous pain and soft tissue infections, often requiring surgical debridement and amputation. Traditionally observed in patients with end-stage kidney disease (ESKD; e.g., ˜1% of hemodialysis patients have calciphylaxis), calciphylaxis is also associated with diabetes, hyperphosphatemia, and warfarin use.⁴ However, the molecular mechanisms of calciphylaxis are incompletely understood, which has precluded the development of approved therapies. Recent investigations have demonstrated that the process of small vessel arteriolar calcification in calciphylaxis exhibits similarities to that of large artery calcification. Coronary and aortic calcification are characterized by the phenotypic switch of vascular smooth muscle cells (VSMCs) from contractile to osteogenic cells, which is induced by Runt-related transcription factor 2 (Run×2) and vitamin K-dependent modulation of matrix Gla protein (MGP) and the bone morphogenetic protein (BMP) signaling pathway. Similarly, in calciphylaxis, there is increased expression of osteogenic markers as well as proteins associated with altered remodeling of the extracellular matrix. Vitamin K deficiency-mediated reduction in carboxylated MGP (a known inhibitor of BMP signaling) is associated with increased risk of calciphylaxis in patients on hemodialysis. Patients with calciphylaxis have reduced pyrophosphate levels compared to matched ESKD patients (unpublished data), implicating ENPP1 in the pathogenesis of calciphylaxis. A treatment as described herein can result in reduced vascular calcification.

Cardiovascular disease: Vascular calcification plays an important role in human arterial disease (e.g., in atherosclerosis and diabetes). Cardiovascular disease is the leading cause of morbidity and mortality in the world. In the United States alone, cardiovascular disease accounts for over 780,000 deaths annually. Vascular calcification is a hallmark of atherosclerotic disease and serves as strong predictor and risk factor for cardiovascular events. Two primary types of vascular calcification have been reported in adults: intimal calcification, associated with atherosclerosis, and medial calcification, associated with chronic kidney disease and diabetes. Intimal calcification occurs in the setting of lipid accumulation and macrophage infiltration into the vessel wall. Medial wall calcification localizes to elastin fibers or smooth muscle cells and is not associated with lipid deposition or macrophage infiltration. Intimal calcification of the atherosclerotic vessel wall is thought to contribute to plaque destabilization and predicts increased risk in cardiovascular disease. Calcification of the medial vessel layer also predicts cardiovascular events and is associated with increased wall stress, pulse pressure, and risk of rupture in aortic aneurysms. Vascular calcification is a tightly regulated process and overlap exists in the molecular underpinnings of atherosclerotic intimal calcification and medial calcification. More recently, common variants (single nucleotide polymorphisms, SNPs) in the ENPP1 locus have been associated with coronary artery calcification at a genomewide level of significance (Kavousi et al., Nat Genet. 2023 October; 55(10):1651-1664). These SNPs that are associated with increased risk of coronary calcification are also associated with lower expression of ENPP1. These findings implicate relative ENPP1 deficiency in the development of calcification and cardiovascular disease in the general population. A treatment as described herein can result in reduced vascular calcification, and reduced risk of cardiovascular disease. Subjects at risk can be identified, e.g., by family history, early stage disease, and/or the presence of SNPs associated with coronary artery calcification.

Calcific Aortic Valve Disease (CAVD): CAVD is the most prevalent cardiac valvular disease among elderly individuals and the prevalence of CAVD is increasing with ˜5% of all individuals above the age of 75 affected. A 3.5-fold increased annual incidence now compared to 30 years ago has been reported.41 CAVD progresses from mild calcification of the valve leaflets to severe calcification and narrowing of the aortic valve orifice, resulting in an obstruction to forward blood flow from the left ventricle and left ventricular hypertrophy. No medical treatment exists for CAVD and treatments are surgical including surgical aortic valve replacement (SAVR) or transcatheter aortic valve implantation (TAVI). An ex vivo study of aortic valve leaflets demonstrated an important role for endogenous pyrophosphate in the inhibition of valvular calcification. Furthermore, a combined proteomic-metabolomic analysis of CAVD vs control aortic valve tissue identified ENPP1 as a hub protein in the metabolite-protein-pathway network. This evidence points towards ENPP1 as a potential therapeutic in aortic valve calcification. Multiple animal models for CAVD exist that overlap with models of atherosclerosis and vascular calcification. A treatment as described herein can result in reduced aortic valve calcification.

Vascular conditions associated with proliferation of SMCs can also be treated using the methods described herein, including in-stent restenosis and graft stenosis (e.g., coronary artery bypass graft stenosis), both of which are due to proliferation of SMCs.

### Pharmaceutical Compositions

Also provided herein are pharmaceutically acceptable compositions that contain a gene therapy construct, e.g., in a viral or non-viral delivery vector, e.g., in an LNP, as described herein, e.g., in a physiologically acceptable carrier. The pharmaceutical compositions can be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile carrier such as a diluent (e.g., sterile water or saline), a fixed oil, polyethylene glycol, glycerin, propylene glycol or other synthetic solvents, antibacterial or antifungal agents such as benzyl alcohol or methyl parabens, chlorobutanol, phenol, ascorbic acid, thimerosal, and the like, antioxidants such as ascorbic acid or sodium bisulfite, chelating agents such as ethylenediaminetetraacetic acid, buffers such as acetates, citrates, or phosphates, and isotonic agents such as sugars (e.g., dextrose), polyalcohols (e.g., mannitol or sorbitol), or salts (e.g., sodium chloride), or any combination thereof. Preparations of the compositions can be formulated and enclosed in ampules, disposable syringes, or multiple dose vials. Where required (as in, for example, injectable formulations), proper fluidity can be maintained by, for example, the use of a coating such as lecithin, or a surfactant. Absorption of the therapeutic nanoparticles can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid.

Any of the compositions described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Dosage, toxicity and therapeutic efficacy of the therapeutic constructs can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Constructs that exhibit high therapeutic indices are preferred. While constructs that exhibit toxic side effects may be used, care should be taken to design a delivery system that targets such constructs to the site of affected tissue in order to minimize potential damage to unaffected cells and, thereby, reduce side effects.

Data obtained from cell culture assays and animal studies can be used in formulating a range of dosage for use in humans. The dosage of such constructs lies preferably within a range of circulating concentrations that include the ED50 with little or no toxicity. The dosage may vary within this range depending upon the dosage form employed and the route of administration utilized. For any construct used in a method described herein, a therapeutically effective dose can be estimated initially from animal models or based on other constructs. Such information can be used to more accurately determine useful doses in humans.

An “effective amount” is an amount sufficient to effect beneficial or desired results. For example, a therapeutic amount is one that achieves a desired therapeutic effect. This amount can be the same or different from a prophylactically effective amount, which is an amount necessary to prevent onset of disease or disease symptoms. An effective amount can be administered in one or more administrations, applications, or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The pharmaceutical compositions can be included in a container, pack, or dispenser together with instructions for administration.

### Exemplary Sequences and Constructs

In some embodiments, the sequence of a protein or nucleic acid used in a composition or method described herein is at least 80%, 85%, 90%, 95%, 97%, 98%, or 99% identical to a reference sequence set forth herein. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can be determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

SECRETORY SEQUENCES

| | | Amino Acid |
|---|---|---|
| | Sequence | Sequence |
| Albumin Secretory | ATGAAGTGGGTAACCTTTATTTCCCTTCTTTTT | MKWVTFISLLFLF |
| Sequence used in | CTCTTTAGCTCGGCTTATTCCAGGGGTGTGTT | SSAYSRGVFRR |
| exemplary | TCGTCGA (SEQ ID NO: 52) | (SEQ ID NO: 53) |
| constructs | | |
| Albumin secretory | ATGAAGTGGGTAACCTTTATTTCCCTTCTTTTT | MKWVTFISLLFLF |
| sequence | CTCTTTAGCTCGGCTTATTCC (SEQ ID NO: 54) | SSAYS (SEQ ID |
| | | NO: 55) |
| ENPP2 Secretory | ATGGCAAGGAGGAGCTCGTTCCAGTCGTGTC | MARRSSFQSCQII |
| Sequence | AGATAATATCCCTGTTCACTTTTGCCGTTGGAG | SLFTFAVGVNICL |
| | TCAATATCTGCTTAGGA (SEQ ID NO: 56) | G (SEQ ID NO: 57) |
| Transthyretin | ATGGCTTCTCATCGTCTGCTCCTCCTCTGCCT | MASHRLLLLCLAG |
| Secretory Sequence | TGCTGGACTGGTATTTGTGTCTGAGGCT (SEQ | LVFVSEA (SEQ ID |
| | ID NO: 58) | NO: 59) |

PROMOTERS

HDAC9-P1 (full length)
ataaatgttttgtagaataaaaaaaaaaaaagttcttcaaaagaaatctcaaatctccaaatggaaacaggta
aaagtggagctcccctggttccacggagaaccttttttgaggaaacttaggcaactcgcaggtaccttatgtc
atgagacagagtttgaaaactacaattgactatctctaaatttcctcccaggtctaaaatgtgatgatagtta
ctacttcagtacatcatccttaaggaaaattattaggtccacactgtttctatcctttgaattttacacataa
attttgtaatcaaaagtttatttgtaatatcagatggaatcagataattgctttttgttttttccactgacag
gaacataagattttgttgtgtagcttaagtcaaacgcagtttggaatatatattttttaaaaattgtaactta
catatccaaatacaatttttcaagaagtagagtattcagtagaaattaatctgtgaaagaagaggaattcagc
agtggcctatttgatgaatgatttaacgtgcttatttcttccctttcatcaaaactctgtgtccccttgtttg
ccccctctgacttcatactctggagttgaccaagatccctcttccatcggattgttctgggaattttgaaata
atctgctttttcctctctttcccctgttgcttctgatgccttagaattacattttcctcgctgatttagttta
gaaaagagaaaagagcttccatgactagtagattatcacttttgggtttgctcttggaagtgacaagatgcta
ggatccctctttggaatgtaaaatttatctcttatatagaaaggatataaatgtagcaccagagactataaaa
ctctgatactatctactgtactgtatagctgaacgccacaatgtgtctggtaatctattgactatcataaatg
ctatttctacagaaaagttaggaggtccatatttcgggcaaccaatgtatagctgaatgcagaacagtcatag
ttgggtactaaccatatatatgatttatccatcaacaggtgcatatgctcagaaattctgtatccataagaaa
tcagactactttcttttccttttgcaagtaaattgaatttagcctgagaggctgaggggaaattttcacatat
aagccacggttttgtgttttgtgttttgttttgtttatagatatagtactaactggatggatgcgataaaatt
cataggtggtactaagatacaataggatttgtgaaatggacaattgtcttgcataaatagcaagtaaaaaatc
aagcctgtccttcataaaaattttattctggggtgtgcttgttttccaaaagtacctctgctaaatctcctgt
tagtcctgaaactagaaggcagaaaagcttctagtgctacagccaactgcagtgtagcctgagaaacaggcaa
caaaaatagaacaccaggattgctgtgcgtgggtgaggcagaaaccacattatgagcaaaagcttccagtatt
attttagaaccaatacagagctctgtacttctctcctctctcttccaaaaacacatactacaaaaataaaatg
aaatgaaatgtatgtgcatttgccctcttagaattatgattcttaattttttttcttgccttcctttctttgg
aagcgaatcgccagtatggaaacacagtgtgtaaagcaagcttcgagagaggaaagagttaattggttttaag
gccctgcgatagagaattatggttggaaagatagaggctggacagctgggtttgctggggtatttttaaatgc
attaatgcaggctccaatcactcggccatgcttgacctatttttggctcaggccgaccattgttctatttctg
tgcctgtgggccatgctgttgttgattcatatgcaaatggattatcactcgctttagccaacttgagctgaga
gagactgagaaagggggaagagaggcacagacacagataggagaagggcaccggctggagccacttgcaggac
tgagggtttttgcaacaaaaccctagcagcctgaagaactctaagccaggtttaattggtttctttttctcgt
gggtagacttaataattttctacgtattctgacaaagaaataaccccgaagcacgttcctatttcccacctgc
ttgtagtttccgggataacctaaactccagagagctatagcatccactctgtcctttctgctttgcacacagg
ttggtaacatgggaaaagtgtccaggtctttttaaaagtggatgcccatttgagcagaaaggaaatcattgtc
gaagttgatcctctgctgcttctcctcagggaggagggagaaccagcgagggtagctcctggggccggtgcac
tgagcagtgatgaatgtttcatgtagctgaagtaagagtgactggaatatgctgcagacaatttacgagagtg
actcctgtttttcctcagATGGGGTGGCTGGACGAGAGCAGCTCTTGGCTCAGCAAAGA (SEQ ID
NO: 60)
HDAC9-P2
ataaatgttttgtagaataaaaaaaaaaaaagttcttcaaaagaaatctcaaatctccaaatggaaacaggta
aaagtggagctcccctggttccacggagaaccttttttgaggaaacttaggcaactcgcaggtaccttatgtc
atgagacagagtttgaaaactacaattgactatctctaaatttcctcccaggtctaaaatgtgatgatagtta
ctacttcagtacatcatccttaaggaaaattattaggtccacactgtttctatcctttgaattttacacataa
attttgtaatcaaaagtttatttgtaatatcagatggaatcagataattgctttttgttttttccactgacag
gaacataagattttgttgtgtagcttaagtcaaacgcagtttggaatatatattttttaaaaattgtaactta
catatccaaatacaatttttcaagaagtagagtattcagtagaaattaatctgtgaaagaagaggaattcagc
agtggcctatttgatgaatgatttaacgtgcttatttcttccctttcatcaaaactctgtgtccccttgtttg
ccccctctgacttcatactctggagttgaccaagatccctcttccatcggattgttctgggaattttgaaata
atctgctttttcctctctttcccctgttgcttctgatgccttagaattacattttcctcgctgatttagttta
gaaaagagaaaagagcttccatgactagtagattatcacttttgggtttgctcttggaagtgacaagatgcta
ggatccctctttggaatgtaaaatttatctcttatatagaaaggatataaatgtagcaccagagactataaaa
ctctgatactatctactgtactgtatagctgaacgccacaatgtgtctggtaatctattgactatcataaatg
ctatttctacagaaaagttaggaggtccatatttcgggcaaccaatgtatagctgaatgcagaacagtcatag
ttgggtactaaccatatatatgatttatccatcaacaggtgcatatgctcagaaattctgtatccataagaaa
tcagactactttcttttccttttgcaagtaaattgaatttagcctgagaggctgaggggaaattttcacatat
aagccacggttttgtgttttgtgttttgttttgtttatagatatagtactaactggatggatgcgataaaatt
cataggtggtactaagatacaataggatttgtgaaatggacaattgtcttgcataaatagcaagtaaaaaatc
aagcctgtccttcataaaaattttattctggggtgt (SEQ ID NO: 61)
HDAC9-P3
gcttgttttccaaaagtacctctgctaaatctcctgttagtcctgaaactagaaggcagaaaagcttctagtg
ctacagccaactgcagtgtagcctgagaaacaggcaacaaaaatagaacaccaggattgctgtgcgtgggtga
ggcagaaaccacattatgagcaaaagcttccagtattattttagaaccaatacagagctctgtacttctctcc
tctctcttccaaaaacacatactacaaaaataaaatgaaatgaaatgtatgtgcatttgccctcttagaatta
tgattcttaattttttttcttgccttcctttctttggaagcgaatcgccagtatggaaacacagtgtgtaaag
caagcttcgagagaggaaagagttaattggttttaaggccctgcgatagagaattatggttggaaagatagag
gctggacagctgggtttgctggggtatttttaaatgcattaatgcaggctccaatcactcggccatgcttgac
ctatttttggctcaggccgaccattgttctatttctgtgcctgtgggccatgctgttgttgattcatatgcaa
atggattatcactcgctttagccaacttgagctgagagagactgagaaagggggaagagaggcacagacacag
ataggagaagggcaccggctggagccacttgcaggactgagggtttttgcaacaaaaccctagcagcctgaag
aactctaagccaggtttaattggtttctttttctcgtgggtagacttaataattttctacgtattctgacaaa
gaaataaccccgaagcacgttcctatttcccacctgcttgtagtttccgggataacctaaactccagagagct
atagcatccactctgtcctttctgctttgcacacaggttggtaacatgggaaaagtgtccaggtctttttaaa
agtggatgcccatttgagcagaaaggaaatcattgtcgaagttgatcctctgctgcttctcctcagggaggag
ggagaaccagcgagggtagctcctggggccggtgcactgagcagtgatgaatgtttcatgtagctgaagtaag
agtgactggaatatgctgcagacaatttacgagagtgactcctgtttttcctcagATGGGGTGGCTGGACGAG
AGCAGCTCTTGGCTCAGCAAAGA (SEQ ID NO: 62)
HDAC9-P2.1 RFP
Ataaatgttttgtagaataaaaaaaaaaaaagttcttcaaaagaaatctcaaatctccaaatggaaacaggta
aaagtggagctcccctggttccacggagaaccttttttgaggaaacttaggcaactcgcaggtaccttatgtc
atgagacagagtttgaaaactacaattgactatctctaaatttcctcccaggtctaaaatgtgatgatagtta
ctacttcagtacatcatccttaaggaaaattattaggtccacactgtttctatcctttgaattttacacataa
attttgtaatcaaaagtttatttgtaatatcagatggaatcagataattgctttttgttttttccactgacag
gaacataagattttgttgtgtagcttaagtcaaacgcagtttggaatatatattttttaaaaattgtaa
(SEQ ID NO: 63)
HDAC9-P2.2_RFP
Cttacatatccaaatacaatttttcaagaagtagagtattcagtagaaattaatctgtgaaagaagaggaatt
cagcagtggcctatttgatgaatgatttaacgtgcttatttcttccctttcatcaaaactctgtgtccccttg
tttgccccctctgacttcatactctggagttgaccaagatccctcttccatcggattgttctgggaattttga
aataatctgctttttcctctctttcccctgttgcttctgatgccttagaattacattttcctcgctgatttag
tttagaaaagagaaaagagcttccatgactagtagattatcacttttgggtttgctcttggaagtgacaagat
gctaggatccctctttggaatgtaaaatttatctcttatatagaaaggatataaatgtagcaccagagactat
aaaactctgatactatctactgtactgtatagctgaacgccaca (SEQ ID NO: 64)
HDAC9P2.3_RFP
Atgtgtctggtaatctattgactatcataaatgctatttctacagaaaagttaggaggtccatatttcgggca
accaatgtatagctgaatgcagaacagtcatagttgggtactaaccatatatatgatttatccatcaacaggt
gcatatgctcagaaattctgtatccataagaaatcagactactttcttttccttttgcaagtaaattgaattt
agcctgagaggctgaggggaaattttcacatataagccacggttttgtgttttgtgttttgttttgtttatag
atatagtactaactggatggatgcgataaaattcataggtggtactaagatacaataggatttgtgaaatgga
caattgtcttgcataaatagcaagtaaaaaatcaagcctgtccttcataaaaattttattctggggtgt
(SEQ ID NO: 65)
MiniHDAC9
Gaaaactacaattgactatctctaaattttaaaatgtgatgatagttactacttcagtggtccacactgtttc
tatcctttggaacataagattttgttgtgtagcttaaggaagaggaattcagcagttgactatctcgattgtt
ctgggaattttggaggctgaggggaaattttcacatataagcgctgggtttgctggggtattgagagtgac
(SEQ ID NO: 66)
human matrix Gla protein (hMGP)
aagattatagttgtcatttgaacttggggataaaggagacatctatgacttggctggaaaagacagagctaat
gtacattgcaaagcacatatttatagcaggaaaatgggaagatttctctttaattctggagatggagtgggga
tggggagagtagactactcattttaagggtgaaacattggaattcaacttgtttgatgttatattaattggtg
gttaattactaagctaagtacgtataaaacttttatctatggctagcttgtccccccaaagtcatgcaatata
gtgaactggctttcgcactttaaattattcattgatcatgtaatgattcagatgattcatcttccaagatgga
cactgaaactaacactcatagtaggttgtggtttaaagagtggaacaaccgccagtctcattagtggaaattg
tgatggttgaatttatcaaggatgaacatacacggtcttctttctgagattttctttaagattttcgcacaga
taatctatttcttaggttttggagagaaaacttgaattttattgatccctcagaactcaatctttcagatttc
aaaggagctatttcttttaatggggactctgttaatatttataaaagctcttcacaggatggagggtgggagg
gaaactccatcccaacaagacaaaaagaatgaagcatgaggctccacctagttcatcactgctccttgaaata
catcagtattgaaagacacatccaccccacccccaacccagccctattgctgttccagctcaagagtcagagg
tcccgaagctgtagctcttctacaatctgctgctctgtgacttcaagtctgttgtctgcaaagaaaactattg
ggttcccaagcaagagaggcacatctggtaggacagattttgtgattgcaaaagaagggggaaaaaaagaaag
aaagaaaagacctctctatacaagataaccagaggcatcaaactgaaatcctcctgtggaaaataagctagta
cttctgggcctgatggtgtagtgaaaacctgtgcttgaggatacattacagtgaaagagcaaagtgaatagta
agtagctattacttacctccttagggaggtgtgttgtttgtctgtacatcccccacagcacctagcacagtac
cttgcatctcacctgccactcactaaaaagtctatcaagttagttaattatcgagacaacgccctcagaaatg
agagaacagtaccctcttatccttgctgcactttccagcactgatacgctgcctaaaagaggactagggcaca
ggtttgaattaatgtcacaaaactggatgggcaagttacaacggtgttgattaaggaaacagaactcatggtg
caccggatatctccatcctgatgaacccttggaaaaatgccaaagatgcatatccccaggcaaatgcctgatt
agtctgggattgatagattggtctaggattcagccctactgggaagatgtctaaattataatcagtgtagaaa
gcgaagttctcctagaagaagaggcaaaggttaaaaagaagaaaagaaaagaaagtgaagtcctttctccccc
aaaacctctcatcaatcaatcagggtaacaaacagaacactagggctctgtctgtggaccaaacccaaaagcc
ctgcggtcagggccaggagggtagatcatgtgtttgtggcaacttcctctgtgggcttttgcccaggtctgtc
cccaagcatacgatggccaaaacttctgcaccagagcagcatcctgtgtaacacagtcaggtccagcagttag
ggaaaactgcccactcagagtagataatatctggaaggaatgactgtttgggaaaagttccaatgctagttca
gtgccaacccttccccaccttctccagctctctcccactggttcctcccctctcaactgctctggttcttata
aaaacctcacagccttccactaacatcccGTAGGAGCCTCTCTCCCTACTGCTGCTACACAAGACCCTGAGAC
TGACCTGCAGGACGAAACC (SEQ ID NO: 67)
hMyh11
CGCCGGGGAGCAGGAAGGCCACTCGGCACCATATTTAGTCAGGGGGAGCCGGCAGCCCAGAGCTGGTATGCGG
CGCTGGGAATTCCTGCAGGAAGGAGTCCGCGCCTGCCCTTTTTGGGTTGTCTCCCGCCCGCCGCTCCCGCCGC
TCCCGGGGAGGGGGACCGGCCCGGCCCGGCCCGGCCCGGGAACCTCGGAGGAGCTGGTGCCGCGCGGGGAGCG
GAGCGCCCGGGCTGCCCGCGGGTCCCCGGCCTGGCGCGGGGCCAGCCCACCGCCTCGACTTCCTTTTATGGCC
TGTGTGTGCGTGCGTGGACAGGAGCGGGGAGGGAGGGACGGGGAGAAGACGGAGAGCCTGGGGAAGAGAGAGA
GAGAAAGCGCAGAGATAGGAGTGAGACACGCGGGAGAGATGGAGAGCAAGAGACACAGAGACCAGAGACAAAG
TGAGACAGGAGGGAGAGACAGATACATCGACAGATCTAGAGAAGCGAGAGGGACAGAGACAAAAGATAGAGCG
AGAGACAGCAATGATCAGAGTGACAGACATGCAGAGACAGTGGCAGAGACAGAGCGAGAGAGCCTGTGATGGA
GAGAGACAGGGAATGCAATTTTAGGCGAGGAATCCTTGGGGAAGGGAAGTTGTTGAAGGGAACTCGCAGACTC
TGGGGGCACACCCACTTTCTCCTTGGATCTTGACACTTGCATCTTGTAAATAACGTAATTATCACCGCCACCG
CCTTCCCCCATTTTGTAGCTATGGACACCAAGTCTCAGAGAAGTGAAGTGACTTGCCCAAGGTCACGCAGCTG
GCGAGTGGCGCACAGGGGAGGGGGACAGCTGAAATAATCACAGTGGGCTTATTTTTAATTTTTATTTGTATTT
TGGTCGTGGTGATGTGGGTGGAGGTGGAGATGGCAAGTTGGGAAAAGTAAAAACTTCCCCTTCCTGCACGGTT
CCCAGCAAGGGTGGGGGCCTCCTGTCTTGCACTTTGCAAAGTTCAAGAAATCCCCTTTCCCTACCCTTCACGC
TGCACAGCCGGCCCTCTTTCCAGACAGTGCGATGCCAATAAAATGGGAAGTGGGGTGGGAGATGTCAAGTCAG
ATCCACCACAGCCCCGACACGGGGAGGAAGAGGTTAAAGCCTTTGCGGCCGGAACCGACTCAGGGAAGACGTT
CTCAAGCATCCCGCACAGACACTGCCTGCTCGACCCCCTTTCTCTAGGGATCCGGAGCGTCTGCGACCGCCTG
GGGCCGGGGCTGAGACTCCCGTCCCTGTGCGCACCTGTTCCGTGCGCCCTTGTGCGGTGCGCACCTGTTCCGT
GCACCCTTGTCCCGAGCGCCCCAGCTCCTTGCGCTCCCGCCGGGGGTGCGCCCTGCAGGGGGCGCGGCGAGGG
GGCCGCGAGGGACCCTCCCCAACTCCACCCCTTCGGCCTCCTCCCCTTTCCCAGCCGCGGGCAGCTCCGGGTC
TATAAAGAGAGGCGTCCGAGGACGCGCAGGGAGATTTGGACGCTCCGGCCTGGGAGGTGCGTCAGATCCGAGC
TCGCCATCCAGTTTCCTCTCCACTAGTCCCCCCAGTTGGAGATCTGTAAGTAGTAGTTGTCATTCTGGGGGCA
GATTGCAGGGCAGGGGGGTGTTAAAAGTCCTATAGGGTATTCTATAGGGGCTGGGGTGCACTTAGGGGT
(SEQ ID NO: 68)
ENPP1
ctctcataatattgaagtagctttgttgtctatggtgacatccgaggttcttggtctcacggccacaaagatc
aaatatgtggacacacacaaagggtgaggtttggagcagaatttttaataggcaaaaggaggagaatagctct
gctacagagaggggtcccaaaaaatgggttgcaatacgtggtgaaatgcaggggtttttatagatgagctagt
ggggaggcggtgtctgatctacataaggagggaaaacccggtttcgtgcatatggcatgaaaacccggttagg
accaggtgtgtcatctgcatagggcttaagtgtctggcagccccggccccaatcttctactatataggcaggt
agctactccgtgttgcttatttccttcttactgtgcatgtggtaaaaaagaggggaggtggaacccccatggt
ggacatgcctggccccagtagccccttctgtctgtgccgctgccggcatctcccgtgcaagcttccagtttcc
ttacctatgtttgcagcccaatcttccaggttgctctttgttagaaaggaagtgatttcttgggctgcttttt
gttagaaggtaagttctgccgagggctcttttgctcatactatctgcctaaataatttctatctcctgtatca
atatcctatgtgtgactctattctgcatatatgacttaggttttaattattttgtttacgagttggtcctacc
cccccacccccgcttccaccacacttaaggaaagatacattttaattcattttttatatggtatgcatagttg
gaggccaaaagattgagttgaaggattcattacttgatgaataaatggatgggtggatggatgagtcagtata
aatgaagagcatcccaagttgactattttaaagggataacatgcattttatatttgttaaacattcattctca
tttgtaaacacttgtatatattcatgtattcaaaatgtattgaaaattaattgttttccttggtacctgaagt
cagtcctgggtattgcaaagtagttcgaagtcactggtttccctctgtctcacttcactacacactggtgatt
ttgctaagtgatgagccagaaaatgtacaagttccagaatcaaaggtaaaacaaaacccaccaagaatcagct
cactgttagcgtcctagttgaaaaataaggcttatcaaagtatttcagattgcattaacacttttgatatagc
taggtatcagtgtcaacctcaggagagggcaacgccaactcctgaaaccttcaatacagaaagtctagaaaaa
taagcaaatatacatacaaacaatagcgagaagactccagaaaccacataaaaccgtgggaatcgaaatatcc
cttacgtgtgttcagtacatgtgaacccacgtattttaagtggacgatttctctctcagagtaccgtaggtag
tgggggacggggcgcagagggggagaaacagaaagtcgccttcctccatggttcatttgcatttccatccaga
aactcacaggtcgaccccaagactccactctctcccgcctttgagaagccggaccggcatcggcggctgcatc
cttctcctcctccccgctctattttggggccccatgatctcatgccctctgcagaccacacgctgcaattcca
gcccagcccgcgccgcgaggccacgcagggcgattcctgcaagtgtcgggagggtggccggggcgcggggagg
ggacggcttggggggaagtttaagacacgcccacgtaagggacccaaaataaccgacacacagagtgcccgaa
atcagacaggaagccaaataatccggggcgttgagtcgctttgccctgactgcgagagccgggtgtagggcgg
ggagccaaggatctgaccgcgaggggcgggcgcggcggggaggggcggggcggggcgggcggcgcggggccta
ttaaaggcgcggccgggcagcggggccggAGCGGCCGGGGCCACG (SEQ ID NO: 69)
ENPP2
gaggtggaggctgcagtgagccgagatcgagccattgcactccagcctgggcaacaagagtgaaactccatct
caaaataaacaaataaacaaacaaaaacaaaaaaacaaagaaatagtggctgaggtcaactcttttggatatt
gggtaacaagcagatcaacttggctattacagcttttccccctgcccagctagtctgcatataagagaaaaat
aaatggatgatgttcagctcctgaaccctaactttttttccaagggaaccctcattgtcttctagctaggttt
atacaatcaaatatcagacaaagtcaaaagttgttagactctggcaaggcaaaagaagaggctgtttttgttt
ttagaacatttagagcactgtggcatataatgaaataacaggattgccttgatatttttctttggggtttatt
gttttcctctatcatgtctggcttgtatcttgaaatttaacataaattataggaaaatctttcatggagttaa
catcttttataagccttgctaaataccctactccaaatttgttcagtatggcatacagacaaaagcaagaggc
ttacatataaaaatgtataagcctgtcctctttgcaacagtgcaaaactaaaaggattttagaaaggcaatta
acagtttggcagtatgtgtattttctttttttcaattagatattactagattctaagaatctgtaatgaaacc
caggcctactgtacaaccccctataaacaagtttttgcttttgtgctatcatgttttatttgagcagagccac
tactctgttactaatttgttgcatggtaagtctcgtatttgtttacgcacgaacacatgtgctgcggaagaaa
agatggtcactggacctgctgaggaagaggccaaaaacaaatctttttccctcatttgaatcctgaaggcatg
taatatttaaacaactttcagatttagagcccttgattttttcttttgaaagtgtgctggttgccaaattgct
tagtaaagaaactttagtacccacaatagcctcaaaggacacagtatttggtggctacatttaaccaagaaat
aactagggaaacaattcttgacatttcaattcttgctgccattgagatttccttcctaaattcaattgctaat
aaagcttcccatgccatccccttcccccacccatctgcctctcaaaaaccgccccctcctccgccccgcccga
aacaagctgacagtctttacgggaagaagaggcagagactgaagttattttgtccgtcctcccagggcagaag
gactttatgctgcgtggctggcatttccagtattttgataaaagttacttctctaaacttgaaccacagcatg
gggaaaataatgttatcttttatgttagttacaattttgcgaaatttcatcatgagaaactttcaaggaggca
gaagtagctgagaatgtgatactagggacagggtcgctcaaactgccagcaaaataaatagatgggaaagaca
ggaacgttattaggtttataaatgataaaactttccttgcttaaaaaaataaagtggtgaggttgacgccatt
gaatgactgcattgtaaaccttaaaagcttaaagctggtggaaagcccttgcacagccctgttttcattttta
tcggatgaaatgcatttggtttctttttgacaaaccatgtttttgtgaggaaaggaaaatgaacatgcactgt
tatgggccacggcaatgtaacaaaccctcctgttgccagaggataccacatgaaagtgtctgtgggttagggg
agggacctgtaagggggcggggataagggggatgatagcttaagcctcttaggctcagagctgcgatttgtga
acaccctgtgatgtaatcaagctctggacaaatcagaggagtctgtcaacctcccaggtgggattgcttatag
ttaatagactaaacccagagcctcaaagcAGTGCACTCCGTGAAGGCAAAGAGAACACGCTGCAAAAGGCTTT
CCAAGAATCCTCGAC (SEQ ID NO: 70)
ENPP3
gggaattacctgtagattattattaggagaaaaaaagtgctaattaatgaacagtgtacatggtatatgatca
tgctataaaagtgtttctatatgcataaaatacttctagaagaaaacataacctgaggatattggctacagac
agaaaaaataacaggtaagagaagagaaagagactagttattggatccacccttttatacctgtgcattttga
accatataaatgtattacctattagaaaacaactaaacaaaaaggttaaaacatgactgtattggcattatat
gatccatgtctgagtggcaaatcttatcaccagtggctgacacgcaaacagttctcatttccctacctatagt
gtaccatgagaaggtaaggcactactgcaatgtgagctcttcgaggatgacacatttttgtctttttcacgga
tacattttctggtagctgttctttgttacttttacagtttgtctcctcatttttactataatattttcctatc
cttattatagaatcttggtattcctattgtagcatctttgtatcaatgctgtgccaattcctttatttagcag
aaaaatggaatttgtatccttgacaagtaaagcttaacatatctgttttcaagtgtaccaaataaatccgaat
aggatgaaaatgatctcaattaatctaacataggggaggtttttttttttaaacatctcacactgtgcagtat
caacttgacttctcaacacaggttttccactgaaatatacaaattaaaacaaatggaagccagaataaaaaat
gataacaattttatttctcccttctcaggaagatcttaagagcagcatgaaagtaaatgttaaaagaaaaaaa
aaaaaaaaaagaagaggccaaaaatcatcagctgaatgtattaaaaatgaagcgctcacctgagaaagaccac
cccaaaactgtctgaaggcccccaaacagctaattagttttgttttctcatcttcaggagtatccataaacat
gctaaaaaaataaaaattacattatttgtcattcgttttaaaaaggtaattacacacaatcagaatagtggat
cttataaccttccaacacgaaactaagtaaaatctgacagacacaatcacataaaaacacaaaactctgccac
gcatacactcacccaggaaaagcctcttctataacttccgttttctgtaaaaaaaaaaaaaacaaaatgtaaa
caatgttaattttaaaaaggcgcctcccttagccaagtattacaatgtaacacagggcaagtcaccttaatca
gctctgaaactatcaatacctgtctggcctgtcactgtatcgatttcaaaggatacaatgaacttgacagcag
tttgagaatcactaaacttccccgcatacaacctcggtgtcaaaacaagggaataaaaagggaggcgtgagag
gaggttgcccaggccagtttccttggtcggctgcccccgcgtctccccgtccccaagccgtagtcctggatgg
ccggcagctgcactcaccaccacctcttcgaaaatgctctgcagttgcgtctccatctgtactatcacccccg
cctttccagggtgcccggcaaggcccggatcagactcgagctctgggaatataggggcagaggggcggagacc
tctggaggaaaccgtagctcctcggcgtcgcttcctcccccagcgctttacctggagcgttccctcccgagcc
cagccaacagcaggaacctgtacggaagacgggaagggcccggtacgcgccgtttgcaaaccccgcagaaacc
agcggcgccaccagaaggttccgtctgtggagaagggcggcccgcaagccggacgagagcgcccccaaacgga
accttgagcccaaggaccccggaggcatcgtcgaccgcaggccgccacctcccggctggagaaggagttgctt
cctgttcggggctaacccgcttgcagactGAGAAATGAAAAGTTTGGAACCCAGAAGCTGCAGCCTGCGGAAA
ATGCTGGAGCCGCAGCCGACACCAAGTTGATGTCTGTGAGCAGCTGACCTTCTGGACCTCCATCAGCGGCCTG
CAGCCAGGAGCTGAAGCCACAGAGACGGTGTCTTCTCAGCTATGATAACCTCAGTGCAAATATTGCCCAATCA
CTGAAGAGCTGTGATCTTCCTAAACAGTTAAAAGTCAGGCACAGCTATGTAACTCATACAGTTTCTCTTTGCC
AGACTAGACTAAAGAAGGAGCACTAATTTATTCTGATAAAACAGGTCTATGCAGCTACCAGGACA (SEQ
ID NO: 71)
CBA
TCGAGGTGAGCCCCACGTTCTGCTTCACTCTCCCCATCTCCCCCCCCTCCCCACCCCCAATTTTGTATTTATT
TATTTTTTAATTATTTTGTGCAGCGATGGGGGCGGGGGGGGGGGGGGGGGGGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTT
CCTTTTATGGCGAGGCGGCGGCGGCGGCGGCCCTATAAAAAGCGAAGCGCGCGGCGGGCG (SEQ ID
NO: 72)
CMV
GACATTGATTATTGACTAGTTATTAATAGTAATCAATTACGGGGTCATTAGTTCATAGCCCATATATGGAGTT
CCGCGTTACATAACTTACGGTAAATGGCCCGCCTGGCTGACCGCCCAACGACCCCCGCCCATTGACGTCAATA
ATGACGTATGTTCCCATAGTAACGCCAATAGGGACTTTCCATTGACGTCAATGGGTGGAGTATTTACGGTAAA
CTGCCCACTTGGCAGTACATCAAGTGTATCATATGCCAAGTACGCCCCCTATTGACGTCAATGACGGTAAATG
GCCCGCCTGGCATTATGCCCAGTACATGACCTTATGGGACTTTCCTACTTGGCAGTACATCTACGTATTAGTC
ATCGCTATTACCATG (SEQ ID NO: 73)
hALB
AGTTCCAGATGGTAAATATACACAAGGGATTTAGTCAAACAATTTTTTGGCAAGAATATTATGAATTTTGTAA
TCGGTTGGCAGCCAATGAAATACAAAGATGAGTCTAGTTAATAATCTACAATTATTGGTTAAAGAAGTATATT
AGTGCTAATTTCCCTCCGTTTGTCCTAGCTTTTCTCTTCTGTCAACCCCACACGCCTTTGGCAC (SEQ ID
NO: 74)
CAG
GCGTTACATAACTTACGGTAAATGGCCCGCCTGGCTGACCGCCCAACGACCCCCGCCCATTGACGTCAATAAT
GACGTATGTTCCCATAGTAACGCCAATAGGGACTTTCCATTGACGTCAATGGGTGGAGTATTTACGGTAAACT
GCCCACTTGGCAGTACATCAAGTGTATCATATGCCAAGTACGCCCCCTATTGACGTCAATGACGGTAAATGGC
CCGCCTGGCATTATGCCCAGTACATGACCTTATGGGACTTTCCTACTTGGCAGTACATCTACGTATTAGTCAT
CGCTATTACCATGGTCGAGGTGAGCCCCACGTTCTGCTTCACTCTCCCCATCTCCCCCCCCTCCCCACCCCCA
ATTTTGTATTTATTTATTTTTTAATTATTTTGTGCAGCGATGGGGGCGGGGGGGGGGGGGGGGCGCGCGCCAG
GCGGGGCGGGGCGGGGCGAGGGGCGGGGCGGGGCGAGGCGGAGAGGTGCGGCGGCAGCCAATCAGAGCGGCGC
GCTCCGAAAGTTTCCTTTTATGGCGAGGCGGCGGCGGCGGCGGCCCTATAAAAAGCGAAGCGCGCGGCGGGCG
(SEQ ID NO: 75)
hHAMP
tgtgtgtgtgtgttttaattttctttatggaaaaattgacaaaaaaaaaatagagagagaggtatttaactgc
aataaactggccccatgtggcccccgccttgtctgcttgtgtgtttgtccatctcaggagtggggagggggcc
tggggtctgcagagctccacgaggcatggttctgctgttgtgcacatggctgtgcatggtccctgccagctgc
accacccattacccagtggttggttggatggatggaggaattaaggaatgaatgtcccctttgaggccctaga
cgtgcatgagggtgtggggagctggggtcaaggacatgtcccatgttggaggagaggcaggggtctccgtgtc
aacagttcctgaaaacacaaccagcccctggccctgccctgctgggccaaagccctcccctctgcaccagcca
atagtggggcctggccttgagcccctcacccccagggagggcagatggccagggcgccaagcttggcccgtca
gcctgtcgccttgcaccaaggctctggcgcctgtgctgtgacccctgcccctgctgatgatgaaacctgtcct
cagctgagatgcagcgatgcctggtagggctgggggctgctcctgtgtctccccaggtgagcacacccctatt
cactgggccctgcttcagcctgcagcacccttcaactcccaggagctgggcttgccactctgctcaccttgtg
gagctccatctgcctttcctccccaattcccccactccctgcactcgtctcttcccacaagagccctgtctcc
ttttcctagctattcccatctgaggccatctttattcatttagtttttagagacagggtttcactctcaccca
ggctggggtgcagtggcacacaatcacggctcactgcagccttgaccaactacaggtgcgtagcaccacagcc
aagtttttgtatagatggggtctcgctttgttacccaggctgtgacaagag (SEQ ID NO: 76)
hTAGLN
agagcgtctccatgctatggttgcatttccgttttctatgaatgaatttgcattcaataaacaaccagactca
gttcttggggcccttgtttgcactccctctgggtggagctgttgaggatgaggggagaggcggaggtcttcca
tttccccattcttcaagccatggccctactgggaactgcaattccttgattctcccgtttttcctgtccctcc
agcaacagcattaattcagtaaacatttaccggggcactgtgctggacagaggccagttcctggaaaagcctt
tcccacgccatcccactgcagacatccctccttacctccccaggaacagcagtctctgcccacctggccccgc
ccaccagactgaggctcacttcacctctgacctgagcggcccccagctcaccaagccacaggcccaagcagtg
ctccctgatgcggcgtttataatccgctcagcgtgcaggccgaggcaggagggtgatgaaagctgggcaggct
ccaagaggagggagttttgatatgtccctgaaagattcatttagacttcagtcggctaaggaggacatgattt
gggggccaaggaatctgttgaattcagaacacaaccagaggtctgcagggtcagggatggaggagtgggcttt
cccctcgccagggcccactcctcttcctgcttttcctgcaggcgccactgggaggtgctatggctgtgcctcc
cctgggctctggagcatgtccagttgcagtgggcagaactgcggaggcgggcccctcctctgccaggcctggc
agccccctcctagggccttgtttggctaggggtggtgccgggtgtggcagtgtgtgtgtagtggagagtgtta
ggtcttccctaccagatgcccttgcaggggagtgccacagcagtcagtccagggatcccactgttagtctcac
cttttttaacctcttatctctccccaagatccctgaagccaggtacgagcaagatgagagtgggttatctctg
gagtgacagaggctggtctgttttccaggctggtagggactgttcctaaagggaggaagggatgataccagcc
tcctgagcctccttctcctgcgttagtgtctcaggccctgccaggccttatagaccctcttattgacactgcc
cactggatggggaccggagttggactcagcttctgccgaaccctcaaatcccagccccaactaaagcatataa
ctcaagacctacctgcactgaaagctcttctcaacctgagcagggtggtccaattgaaagggtgggtctgacc
acctctcctgcacccatgcgggttggcagaggtgtgcaggatctgccacttaccattcaccatgtggccttga
ggaagacgcactcggggcctcagtttcctcatctataaaatggggatgtaattacaccctcacactgtagctg
tgagtattcaatgagagcactgcaaagggcctggtgtggagtaggtcctcaggaaaggttggatcccatgtcc
catcagagctaaaagccccaggaggagagggtggctggtttgtccccacaaacccctgggattcccggctccc
cagccccttgcccctctctccagccagactctattgaactccccctcttctcaaactcggggccagagaacag
tgaagtaggagcagccgtaagtccgggcagggtcctgtccataaaaggcttttcccgggccggctccccgccg
gcagcgtgccccgccccggcccgctccatctccaaagcatgcagagaatgtctcggcagccccggtagactgc
tccaacttggtgtctttccccaaatatggagcctgtgtggagtcactgggggagccgggggtggggagcggag
ccggcttcctctagcagggagggggccgaggagcgagccagtgggggaggctgacatcaccacggcggcagcc
ctttaaacccctcacccagccagcgccccATCCTGTCTGTCCGAACCCAGACACAAGTCTTCACTCCTTCCTG
CGAGCCCTGAGGAAGCCTTCTTTCCCCAGAC (SEQ ID NO: 77)

Bone targeting sequences

| Nucleic Acid | SEQ ID NO: | Amino acid | SEQ ID NO: |
|---|---|---|---|
| AAGAATTTCCAGAGCAGAAGCCAC | 22 | KNFQSRSH | 78 |
| AAGAGAAGAACCCCTGTGCGGGAG | 23 | KRRTPVRE | 79 |
| AAGACCTACGCCTCTATGCAGTGG | 24 | KTYASMQW | 80 |
| GATGATGACGACGACGATGACTGC | 25 | DDDDDDDC | 81 |

### Plasmid Sequences Comprising ENPP1 Gene Therapy Constructs Full Length Recombinant ENPP1 Constructs

CMV_Kozak_ENPP1 Full Length_FLAG_pcDNA3.1(+)

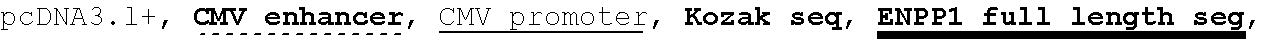

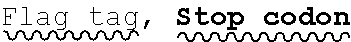

(SEQ ID NO: 82)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG

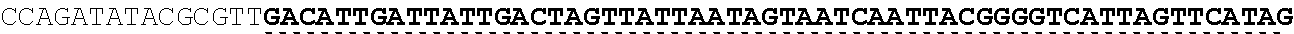

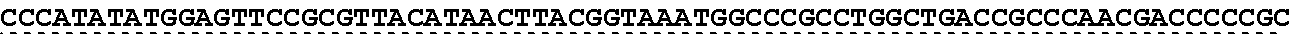

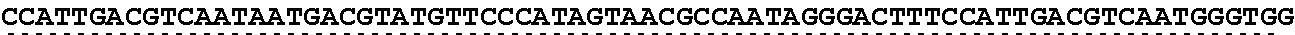

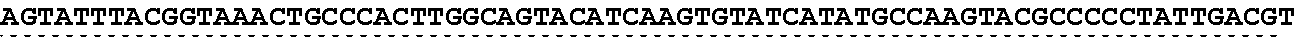

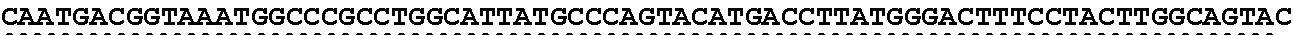

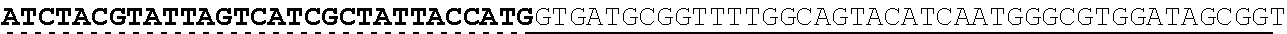

TTGACTCACGGGGATTTCCAAGTCTCCACCCCATTGACGTCAATGGGAGTTTGTTTTGGCACCAAAATCAACG
GGACTTTCCAAAATGTCGTAACAACTCCGCCCCATTGACGCAAATGGGCGGTAGGCGTGTACGGTGGGAGGTC
TATATAAGCAGAGCTCTCTGGCTAACTAGAGAACCCACTGCTTACTGGCTTATCGAAATTAATACGACTCACT
ATAGGGAGACCCAAGCTGGCTAGCGCCACCATGGAGCGCGACGGCTGCGCGGGGGGCGGGAGCCGCGGCGGCG
AGGGCGGGCGCGCTCCCCGGGAGGGCCCGGCGGGGAACGGCCGCGATCGGGGCCGCAGCCACGCTGCCGAGGC
GCCCGGGGACCCGCAGGCCGCGGCGTCCTTGCTGGCCCCTATGGACGTGGGGGAGGAGCCGCTGGAGAAGGCG
GCGCGCGCCCGCACTGCCAAGGACCCCAACACCTATAAAGTACTCTCGCTGGTATTGTCAGTATGTGTGTTAA
CAACAATACTTGGTTGTATATTTGGGTTGAAACCAAGCTGTGCCAAAGAAGTTAAAAGTTGCAAAGGTCGCTG
TTTCGAGAGAACATTTGGGAACTGTCGCTGTGATGCTGCCTGTGTTGAGCTTGGAAACTGCTGTTTAGATTAC
CAGGAGACGTGCATAGAACCAGAACATATATGGACTTGCAACAAATTCAGGTGTGGTGAGAAAAGGTTGACCA
GAAGCCTCTGTGCCTGTTCAGATGACTGCAAGGACAAGGGCGACTGCTGCATCAACTACAGTTCTGTGTGTCA
AGGTGAGAAAAGTTGGGTAGAAGAACCATGTGAGAGCATTAATGAGCCACAGTGCCCAGCAGGGTTTGAAACG
CCTCCTACCCTCTTATTTTCTTTGGATGGATTCAGGGCAGAATATTTACACACTTGGGGTGGACTTCTTCCTG
TTATTAGCAAACTAAAAAAATGTGGAACATATACTAAAAACATGAGACCGGTATATCCAACAAAAACTTTCCC
CAATCACTACAGCATTGTCACCGGATTGTATCCAGAATCTCATGGCATAATCGACAATAAAATGTATGATCCC
AAAATGAATGCTTCCTTTTCACTTAAAAGTAAAGAGAAATTTAATCCTGAGTGGTACAAAGGAGAACCAATTT
GGGTCACAGCTAAGTATCAAGGCCTCAAGTCTGGCACATTTTTCTGGCCAGGATCAGATGTGGAAATTAACGG
AATTTTCCCAGACATCTATAAAATGTATAATGGTTCAGTACCATTTGAAGAAAGGATTTTAGCTGTTCTTCAG
TGGCTACAGCTTCCTAAAGATGAAAGACCACACTTTTACACTCTGTATTTAGAAGAACCAGATTCTTCAGGTC
ATTCATATGGACCAGTCAGCAGTGAAGTCATCAAAGCCTTGCAGAGGGTTGATGGTATGGTTGGTATGCTGAT
GGATGGTCTGAAAGAGCTGAACTTGCACAGATGCCTGAACCTCATCCTTATTTCAGATCATGGCATGGAACAA
GGCAGTTGTAAGAAATACATATATCTGAATAAATATTTGGGGGATGTTAAAAATATTAAAGTTATCTATGGAC
CTGCAGCTCGATTGAGACCCTCTGATGTCCCAGATAAATACTATTCATTTAACTATGAAGGCATTGCCCGAAA
TCTTTCTTGCCGGGAACCAAACCAGCACTTCAAACCTTACCTGAAACATTTCTTACCTAAGCGTTTGCACTTT
GCTAAGAGTGATAGAATTGAGCCCTTGACATTCTATTTGGACCCTCAGTGGCAACTTGCATTGAATCCCTCAG
AAAGGAAATATTGTGGAAGTGGATTTCATGGCTCTGACAATGTATTTTCAAATATGCAAGCCCTCTTTGTTGG
CTATGGACCTGGATTCAAGCATGGCATTGAGGCTGACACCTTTGAAAACATTGAAGTCTATAACTTAATGTGT
GATTTACTGAATTTGACACCGGCTCCTAATAACGGAACTCATGGAAGTCTTAACCACCTTCTAAAGAATCCTG
TTTATACGCCAAAGCATCCCAAAGAAGTGCACCCCCTGGTACAGTGCCCCTTCACAAGAAACCCCAGAGATAA
CCTTGGCTGCTCATGTAACCCTTCGATTTTGCCGATTGAGGATTTTCAAACACAGTTCAATCTGACTGTGGCA
GAAGAGAAGATTATTAAGCATGAAACTTTACCCTATGGAAGACCTAGAGTTCTCCAGAAGGAAAACACCATCT
GTCTTCTTTCCCAGCACCAGTTTATGAGTGGATACAGCCAAGACATCTTAATGCCCCTTTGGACATCCTATAC
CGTGGACAGAAATGACAGTTTCTCTACGGAAGACTTCTCCAACTGTCTGTACCAGGACTTTAGAATTCCTCTT
AGTCCTGTCCATAAATGTTCATTTTATAAAAATAACACCAAAGTGAGTTACGGGTTCCTCTCCCCACCACAAC
TAAATAAAAATTCAAGTGGAATATATTCTGAAGCTTTGCTTACTACAAATATAGTGCCAATGTACCAGAGTTT
TCAAGTTATATGGCGCTACTTTCATGACACCCTACTGCGAAAGTATGCTGAAGAAAGAAATGGTGTCAATGTC
GTCAGTGGTCCTGTGTTTGACTTTGATTATGATGGACGTTGTGATTCCTTAGAGAATCTGAGGCAAAAAAGAA
GAGTCATCCGTAACCAAGAAATTTTGATTCCAACTCACTTCTTTATTGTGCTAACAAGCTGTAAAGATACATC
TCAGACGCCTTTGCACTGTGAAAACCTAGACACCTTAGCTTTCATTTTGCCTCACAGGACTGATAACAGCGAG
AGCTGTGTGCATGGGAAGCATGACTCCTCATGGGTTGAAGAATTGTTAATGTTACACAGAGCACGGATCACAG
ATGTTGAGCACATCACTGGACTCAGCTTCTATCAACAAAGAAAAGAGCCAGTTTCAGACATTTTAAAGTTGAA

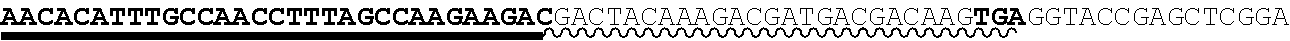

TCCACTAGTCCAGTGTGGTGGAATTCTGCAGATATCCAGCACAGTGGCGGCCGCTCGAGTCTAGAGGGCCCGT
TTAAACCCGCTGATCAGCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATTGCATCGCATTGTCTGA
GTAGGTGTCATTCTATTCTGGGGGGTGGGGTGGGGCAGGACAGCAAGGGGGAGGATTGGGAAGACAATAGCAG
GCATGCTGGGGATGCGGTGGGCTCTATGGCTTCTGAGGCGGAAAGAACCAGCTGGGGCTCTAGGGGGTATCCC
CACGCGCCCTGTAGCGGCGCATTAAGCGCGGCGGGTGTGGTGGTTACGCGCAGCGTGACCGCTACACTTGCCA
GCGCCCTAGCGCCCGCTCCTTTCGCTTTCTTCCCTTCCTTTCTCGCCACGTTCGCCGGCTTTCCCCGTCAAGC
TCTAAATCGGGGGCTCCCTTTAGGGTTCCGATTTAGTGCTTTACGGCACCTCGACCCCAAAAAACTTGATTAG
GGTGATGGTTCACGTAGTGGGCCATCGCCCTGATAGACGGTTTTTCGCCCTTTGACGTTGGAGTCCACGTTCT
TTAATAGTGGACTCTTGTTCCAAACTGGAACAACACTCAACCCTATCTCGGTCTATTCTTTTGATTTATAAGG
GATTTTGCCGATTTCGGCCTATTGGTTAAAAAATGAGCTGATTTAACAAAAATTTAACGCGAATTAATTCTGT
GGAATGTGTGTCAGTTAGGGTGTGGAAAGTCCCCAGGCTCCCCAGCAGGCAGAAGTATGCAAAGCATGCATCT
CAATTAGTCAGCAACCAGGTGTGGAAAGTCCCCAGGCTCCCCAGCAGGCAGAAGTATGCAAAGCATGCATCTC
AATTAGTCAGCAACCATAGTCCCGCCCCTAACTCCGCCCATCCCGCCCCTAACTCCGCCCAGTTCCGCCCATT
CTCCGCCCCATGGCTGACTAATTTTTTTTATTTATGCAGAGGCCGAGGCCGCCTCTGCCTCTGAGCTATTCCA
GAAGTAGTGAGGAGGCTTTTTTGGAGGCCTAGGCTTTTGCAAAAAGCTCCCGGGAGCTTGTATATCCATTTTC
GGATCTGATCAAGAGACAGGATGAGGATCGTTTCGCATGATTGAACAAGATGGATTGCACGCAGGTTCTCCGG
CCGCTTGGGTGGAGAGGCTATTCGGCTATGACTGGGCACAACAGACAATCGGCTGCTCTGATGCCGCCGTGTT
CCGGCTGTCAGCGCAGGGGCGCCCGGTTCTTTTTGTCAAGACCGACCTGTCCGGTGCCCTGAATGAACTGCAG
GACGAGGCAGCGCGGCTATCGTGGCTGGCCACGACGGGCGTTCCTTGCGCAGCTGTGCTCGACGTTGTCACTG
AAGCGGGAAGGGACTGGCTGCTATTGGGCGAAGTGCCGGGGCAGGATCTCCTGTCATCTCACCTTGCTCCTGC
CGAGAAAGTATCCATCATGGCTGATGCAATGCGGCGGCTGCATACGCTTGATCCGGCTACCTGCCCATTCGAC
CACCAAGCGAAACATCGCATCGAGCGAGCACGTACTCGGATGGAAGCCGGTCTTGTCGATCAGGATGATCTGG
ACGAAGAGCATCAGGGGCTCGCGCCAGCCGAACTGTTCGCCAGGCTCAAGGCGCGCATGCCCGACGGCGAGGA
TCTCGTCGTGACCCATGGCGATGCCTGCTTGCCGAATATCATGGTGGAAAATGGCCGCTTTTCTGGATTCATC
GACTGTGGCCGGCTGGGTGTGGCGGACCGCTATCAGGACATAGCGTTGGCTACCCGTGATATTGCTGAAGAGC
TTGGCGGCGAATGGGCTGACCGCTTCCTCGTGCTTTACGGTATCGCCGCTCCCGATTCGCAGCGCATCGCCTT
CTATCGCCTTCTTGACGAGTTCTTCTGAGCGGGACTCTGGGGTTCGAAATGACCGACCAAGCGACGCCCAACC
TGCCATCACGAGATTTCGATTCCACCGCCGCCTTCTATGAAAGGTTGGGCTTCGGAATCGTTTTCCGGGACGC
CGGCTGGATGATCCTCCAGCGCGGGGATCTCATGCTGGAGTTCTTCGCCCACCCCAACTTGTTTATTGCAGCT
TATAATGGTTACAAATAAAGCAATAGCATCACAAATTTCACAAATAAAGCATTTTTTTCACTGCATTCTAGTT
GTGGTTTGTCCAAACTCATCAATGTATCTTATCATGTCTGTATACCGTCGACCTCTAGCTAGAGCTTGGCGTA
ATCATGGTCATAGCTGTTTCCTGTGTGAAATTGTTATCCGCTCACAATTCCACACAACATACGAGCCGGAAGC
ATAAAGTGTAAAGCCTGGGGTGCCTAATGAGTGAGCTAACTCACATTAATTGCGTTGCGCTCACTGCCCGCTT
TCCAGTCGGGAAACCTGTCGTGCCAGCTGCATTAATGAATCGGCCAACGCGCGGGGAGAGGCGGTTTGCGTAT
TGGGCGCTCTTCCGCTTCCTCGCTCACTGACTCGCTGCGCTCGGTCGTTCGGCTGCGGCGAGCGGTATCAGCT
CACTCAAAGGCGGTAATACGGTTATCCACAGAATCAGGGGATAACGCAGGAAAGAACATGTGAGCAAAAGGCC
AGCAAAAGGCCAGGAACCGTAAAAAGGCCGCGTTGCTGGCGTTTTTCCATAGGCTCCGCCCCCCTGACGAGCA
TCACAAAAATCGACGCTCAAGTCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTGTAGGTCGTTCGCTCCAAGCTGGG
CTGTGTGCACGAACCCCCCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAGCGAGGTATGTAGGCGGTG
CTACAGAGTTCTTGAAGTGGTGGCCTAACTACGGCTACACTAGAAGAACAGTATTTGGTATCTGCGCTCTGCT
GAAGCCAGTTACCTTCGGAAAAAGAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAACTTGGTCTGACAGT
TACCAATGCTTAATCAGTGAGGCACCTATCTCAGCGATCTGTCTATTTCGTTCATCCATAGTTGCCTGACTCC
CCGTCGTGTAGATAACTACGATACGGGAGGGCTTACCATCTGGCCCCAGTGCTGCAATGATACCGCGAGACCC
ACGCTCACCGGCTCCAGATTTATCAGCAATAAACCAGCCAGCCGGAAGGGCCGAGCGCAGAAGTGGTCCTGCA
ACTTTATCCGCCTCCATCCAGTCTATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCGTTTGGTATGGCTTCATTCAGCTC
CGGTTCCCAACGATCAAGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCACTGCATAATTCTCTTA
CTGTCATGCCATCCGTAAGATGCTTTTCTGTGACTGGTGAGTACTCAACCAAGTCATTCTGAGAATAGTGTAT
GCGGCGACCGAGTTGCTCTTGCCCGGCGTCAATACGGGATAATACCGCGCCACATAGCAGAACTTTAAAAGTG
CTCATCATTGGAAAACGTTCTTCGGGGCGAAAACTCTCAAGGATCTTACCGCTGTTGAGATCCAGTTCGATGT
AACCCACTCGTGCACCCAACTGATCTTCAGCATCTTTTACTTTCACCAGCGTTTCTGGGTGAGCAAAAACAGG
AAGGCAAAATGCCGCAAAAAAGGGAATAAGGGCGACACGGAAATGTTGAATACTCATACTCTTCCTTTTTCAA
TATTATTGAAGCATTTATCAGGGTTATTGTCTCATGAGCGGATACATATTTGAATGTATTTAGAAAAATAAAC
AAATAGGGGTTCCGCGCACATTTCCCCGAAAAGTGCCACCTGACGTC
CAG_Kozak_ENPP1 Full Length_FLAG_pcDNA3.1 (+)

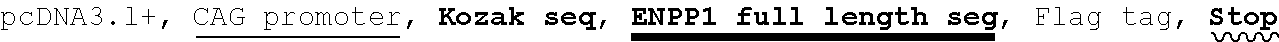

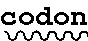

(SEQ ID NO: 83)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTGCGTTACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCATTGACGTCAATGGGTGGAG
TATTTACGGTAAACTGCCCACTTGGCAGTACATCAAGTGTATCATATGCCAAGTACGCCCCCTATTGACGTCA
ATGACGGTAAATGGCCCGCCTGGCATTATGCCCAGTACATGACCTTATGGGACTTTCCTACTTGGCAGTACAT
CTACGTATTAGTCATCGCTATTACCATGGTCGAGGTGAGCCCCACGTTCTGCTTCACTCTCCCCATCTCCCCC
CCCTCCCCACCCCCAATTTTGTATTTATTTATTTTTTAATTATTTTGTGCAGCGATGGGGGCGGGGGGGGGGG
GGGGGCGCGCGCCAGGCGGGGCGGGGCGGGGCGAGGGGCGGGGCGGGGCGAGGCGGAGAGGTGCGGCGGCAGC
CAATCAGAGCGGCGCGCTCCGAAAGTTTCCTTTTATGGCGAGGCGGCGGCGGCGGCGGCCCTATAAAAAGCGA
AGCGCGCGGCGGGCGCTCTGGCTAACTAGAGAACCCACTGCTTACTGGCTTATCGAAATTTAATACGACTCACT
ATAGGGAGACCCAAGCTGGCTAGCGCCACCATGGAGCGCGACGGCTGCGCGGGGGGCGGGAGCCGCGGCGGCG
AGGGCGGGCGCGCTCCCCGGGAGGGCCCGGCGGGGAACGGCCGCGATCGGGGCCGCAGCCACGCTGCCGAGGC
GCCCGGGGACCCGCAGGCCGCGGCGTCCTTGCTGGCCCCTATGGACGTGGGGGAGGAGCCGCTGGAGAAGGCG
GCGCGCGCCCGCACTGCCAAGGACCCCAACACCTATAAAGTACTCTCGCTGGTATTGTCAGTATGTGTGTTAA
CAACAATACTTGGTTGTATATTTGGGTTGAAACCAAGCTGTGCCAAAGAAGTTAAAAGTTGCAAAGGTCGCTG
TTTCGAGAGAACATTTGGGAACTGTCGCTGTGATGCTGCCTGTGTTGAGCTTGGAAACTGCTGTTTAGATTAC
CAGGAGACGTGCATAGAACCAGAACATATATGGACTTGCAACAAATTCAGGTGTGGTGAGAAAAGGTTGACCA
GAAGCCTCTGTGCCTGTTCAGATGACTGCAAGGACAAGGGCGACTGCTGCATCAACTACAGTTCTGTGTGTCA
AGGTGAGAAAAGTTGGGTAGAAGAACCATGTGAGAGCATTAATGAGCCACAGTGCCCAGCAGGGTTTGAAACG
CCTCCTACCCTCTTATTTTCTTTGGATGGATTCAGGGCAGAATATTTACACACTTGGGGTGGACTTCTTCCTG
TTATTAGCAAACTAAAAAAATGTGGAACATATACTAAAAACATGAGACCGGTATATCCAACAAAAACTTTCCC
CAATCACTACAGCATTGTCACCGGATTGTATCCAGAATCTCATGGCATAATCGACAATAAAATGTATGATCCC
AAAATGAATGCTTCCTTTTCACTTAAAAGTAAAGAGAAATTTAATCCTGAGTGGTACAAAGGAGAACCAATTT
GGGTCACAGCTAAGTATCAAGGCCTCAAGTCTGGCACATTTTTCTGGCCAGGATCAGATGTGGAAATTAACGG
AATTTTCCCAGACATCTATAAAATGTATAATGGTTCAGTACCATTTGAAGAAAGGATTTTAGCTGTTCTTCAG
TGGCTACAGCTTCCTAAAGATGAAAGACCACACTTTTACACTCTGTATTTAGAAGAACCAGATTCTTCAGGTC
ATTCATATGGACCAGTCAGCAGTGAAGTCATCAAAGCCTTGCAGAGGGTTGATGGTATGGTTGGTATGCTGAT
GGATGGTCTGAAAGAGCTGAACTTGCACAGATGCCTGAACCTCATCCTTATTTCAGATCATGGCATGGAACAA
GGCAGTTGTAAGAAATACATATATCTGAATAAATATTTGGGGGATGTTAAAAATATTAAAGTTATCTATGGAC
CTGCAGCTCGATTGAGACCCTCTGATGTCCCAGATAAATACTATTCATTTAACTATGAAGGCATTGCCCGAAA
TCTTTCTTGCCGGGAACCAAACCAGCACTTCAAACCTTACCTGAAACATTTCTTACCTAAGCGTTTGCACTTT
GCTAAGAGTGATAGAATTGAGCCCTTGACATTCTATTTGGACCCTCAGTGGCAACTTGCATTGAATCCCTCAG
AAAGGAAATATTGTGGAAGTGGATTTCATGGCTCTGACAATGTATTTTCAAATATGCAAGCCCTCTTTGTTGG
CTATGGACCTGGATTCAAGCATGGCATTGAGGCTGACACCTTTGAAAACATTGAAGTCTATAACTTAATGTGT
GATTTACTGAATTTGACACCGGCTCCTAATAACGGAACTCATGGAAGTCTTAACCACCTTCTAAAGAATCCTG
TTTATACGCCAAAGCATCCCAAAGAAGTGCACCCCCTGGTACAGTGCCCCTTCACAAGAAACCCCAGAGATAA
CCTTGGCTGCTCATGTAACCCTTCGATTTTGCCGATTGAGGATTTTCAAACACAGTTCAATCTGACTGTGGCA
GAAGAGAAGATTATTAAGCATGAAACTTTACCCTATGGAAGACCTAGAGTTCTCCAGAAGGAAAACACCATCT
GTCTTCTTTCCCAGCACCAGTTTATGAGTGGATACAGCCAAGACATCTTAATGCCCCTTTGGACATCCTATAC
CGTGGACAGAAATGACAGTTTCTCTACGGAAGACTTCTCCAACTGTCTGTACCAGGACTTTAGAATTCCTCTT
AGTCCTGTCCATAAATGTTCATTTTATAAAAATAACACCAAAGTGAGTTACGGGTTCCTCTCCCCACCACAAC
TAAATAAAAATTCAAGTGGAATATATTCTGAAGCTTTGCTTACTACAAATATAGTGCCAATGTACCAGAGTTT
AGCTGTGTGCATGGGAAGCATGACTCCTCATGGGTTGAAGAATTGTTAATGTTACACAGAGCACGGATCACAG
ATGTTGAGCACATCACTGGACTCAGCTTCTATCAACAAAGAAAAGAGCCAGTTTCAGACATTTTAAAGTTGAA

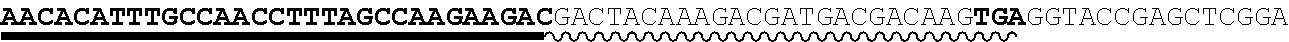

TCCACTAGTCCAGTGTGGTGGAATTCTGCAGATATCCAGCACAGTGGCGGCCGCTCGAGTCTAGAGGGCCCGT
TTAAACCCGCTGATCAGCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATTGCATCGCATTGTCTGA
GTAGGTGTCATTCTATTCTGGGGGGTGGGGTGGGGCAGGACAGCAAGGGGGAGGATTGGGAAGACAATAGCAG
GCATGCTGGGGATGCGGTGGGCTCTATGGCTTCTGAGGCGGAAAGAACCAGCTGGGGCTCTAGGGGGTATCCC
CACGCGCCCTGTAGCGGCGCATTAAGCGCGGCGGGTGTGGTGGTTACGCGCAGCGTGACCGCTACACTTGCCA
GCGCCCTAGCGCCCGCTCCTTTCGCTTTCTTCCCTTCCTTTCTCGCCACGTTCGCCGGCTTTCCCCGTCAAGC
TCTAAATCGGGGGCTCCCTTTAGGGTTCCGATTTAGTGCTTTACGGCACCTCGACCCCAAAAAACTTGATTAG
GGTGATGGTTCACGTAGTGGGCCATCGCCCTGATAGACGGTTTTTCGCCCTTTGACGTTGGAGTCCACGTTCT
TTAATAGTGGACTCTTGTTCCAAACTGGAACAACACTCAACCCTATCTCGGTCTATTCTTTTGATTTATAAGG
GATTTTGCCGATTTCGGCCTATTGGTTAAAAAATGAGCTGATTTAACAAAAATTTAACGCGAATTAATTCTGT
GGAATGTGTGTCAGTTAGGGTGTGGAAAGTCCCCAGGCTCCCCAGCAGGCAGAAGTATGCAAAGCATGCATCT
CAATTAGTCAGCAACCAGGTGTGGAAAGTCCCCAGGCTCCCCAGCAGGCAGAAGTATGCAAAGCATGCATCTC
AATTAGTCAGCAACCATAGTCCCGCCCCTAACTCCGCCCATCCCGCCCCTAACTCCGCCCAGTTCCGCCCATT
CTCCGCCCCATGGCTGACTAATTTTTTTTATTTATGCAGAGGCCGAGGCCGCCTCTGCCTCTGAGCTATTCCA
GAAGTAGTGAGGAGGCTTTTTTGGAGGCCTAGGCTTTTGCAAAAAGCTCCCGGGAGCTTGTATATCCATTTTC
GGATCTGATCAAGAGACAGGATGAGGATCGTTTCGCATGATTGAACAAGATGGATTGCACGCAGGTTCTCCGG
CCGCTTGGGTGGAGAGGCTATTCGGCTATGACTGGGCACAACAGACAATCGGCTGCTCTGATGCCGCCGTGTT
CCGGCTGTCAGCGCAGGGGCGCCCGGTTCTTTTTGTCAAGACCGACCTGTCCGGTGCCCTGAATGAACTGCAG
GACGAGGCAGCGCGGCTATCGTGGCTGGCCACGACGGGCGTTCCTTGCGCAGCTGTGCTCGACGTTGTCACTG
AAGCGGGAAGGGACTGGCTGCTATTGGGCGAAGTGCCGGGGCAGGATCTCCTGTCATCTCACCTTGCTCCTGC
CGAGAAAGTATCCATCATGGCTGATGCAATGCGGCGGCTGCATACGCTTGATCCGGCTACCTGCCCATTCGAC
CACCAAGCGAAACATCGCATCGAGCGAGCACGTACTCGGATGGAAGCCGGTCTTGTCGATCAGGATGATCTGG
ACGAAGAGCATCAGGGGCTCGCGCCAGCCGAACTGTTCGCCAGGCTCAAGGCGCGCATGCCCGACGGCGAGGA
TCTCGTCGTGACCCATGGCGATGCCTGCTTGCCGAATATCATGGTGGAAAATGGCCGCTTTTCTGGATTCATC
GACTGTGGCCGGCTGGGTGTGGCGGACCGCTATCAGGACATAGCGTTGGCTACCCGTGATATTGCTGAAGAGC
TTGGCGGCGAATGGGCTGACCGCTTCCTCGTGCTTTACGGTATCGCCGCTCCCGATTCGCAGCGCATCGCCTT
CTATCGCCTTCTTGACGAGTTCTTCTGAGCGGGACTCTGGGGTTCGAAATGACCGACCAAGCGACGCCCAACC
TGCCATCACGAGATTTCGATTCCACCGCCGCCTTCTATGAAAGGTTGGGCTTCGGAATCGTTTTCCGGGACGC
CGGCTGGATGATCCTCCAGCGCGGGGATCTCATGCTGGAGTTCTTCGCCCACCCCAACTTGTTTATTGCAGCT
TATAATGGTTACAAATAAAGCAATAGCATCACAAATTTCACAAATAAAGCATTTTTTTCACTGCATTCTAGTT
GTGGTTTGTCCAAACTCATCAATGTATCTTATCATGTCTGTATACCGTCGACCTCTAGCTAGAGCTTGGCGTA
ATCATGGTCATAGCTGTTTCCTGTGTGAAATTGTTATCCGCTCACAATTCCACACAACATACGAGCCGGAAGC
ATAAAGTGTAAAGCCTGGGGTGCCTAATGAGTGAGCTAACTCACATTAATTGCGTTGCGCTCACTGCCCGCTT
TCCAGTCGGGAAACCTGTCGTGCCAGCTGCATTAATGAATCGGCCAACGCGCGGGGAGAGGCGGTTTGCGTAT
TGGGCGCTCTTCCGCTTCCTCGCTCACTGACTCGCTGCGCTCGGTCGTTCGGCTGCGGCGAGCGGTATCAGCT
CACTCAAAGGCGGTAATACGGTTATCCACAGAATCAGGGGATAACGCAGGAAAGAACATGTGAGCAAAAGGCC
AGCAAAAGGCCAGGAACCGTAAAAAGGCCGCGTTGCTGGCGTTTTTCCATAGGCTCCGCCCCCCTGACGAGCA
TCACAAAAATCGACGCTCAAGTCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTGTAGGTCGTTCGCTCCAAGCTGGG
CTGTGTGCACGAACCCCCCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAGCGAGGTATGTAGGCGGTG
CTACAGAGTTCTTGAAGTGGTGGCCTAACTACGGCTACACTAGAAGAACAGTATTTGGTATCTGCGCTCTGCT
GAAGCCAGTTACCTTCGGAAAAAGAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAACTTGGTCTGACAGT
TACCAATGCTTAATCAGTGAGGCACCTATCTCAGCGATCTGTCTATTTCGTTCATCCATAGTTGCCTGACTCC
CCGTCGTGTAGATAACTACGATACGGGAGGGCTTACCATCTGGCCCCAGTGCTGCAATGATACCGCGAGACCC
ACGCTCACCGGCTCCAGATTTATCAGCAATAAACCAGCCAGCCGGAAGGGCCGAGCGCAGAAGTGGTCCTGCA
ACTTTATCCGCCTCCATCCAGTCTATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCGTTTGGTATGGCTTCATTCAGCTC
CGGTTCCCAACGATCAAGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCACTGCATAATTCTCTTA
CTGTCATGCCATCCGTAAGATGCTTTTCTGTGACTGGTGAGTACTCAACCAAGTCATTCTGAGAATAGTGTAT
GCGGCGACCGAGTTGCTCTTGCCCGGCGTCAATACGGGATAATACCGCGCCACATAGCAGAACTTTAAAAGTG
CTCATCATTGGAAAACGTTCTTCGGGGCGAAAACTCTCAAGGATCTTACCGCTGTTGAGATCCAGTTCGATGT
AACCCACTCGTGCACCCAACTGATCTTCAGCATCTTTTACTTTCACCAGCGTTTCTGGGTGAGCAAAAACAGG
AAGGCAAAATGCCGCAAAAAAGGGAATAAGGGCGACACGGAAATGTTGAATACTCATACTCTTCCTTTTTCAA
TATTATTGAAGCATTTATCAGGGTTATTGTCTCATGAGCGGATACATATTTGAATGTATTTAGAAAAATAAAC
AAATAGGGGTTCCGCGCACATTTCCCCGAAAAGTGCCACCTGACGTC
hMyh11_Kozak_ENPP1 Full Length_FLAG_pcDNA3.1 (+)

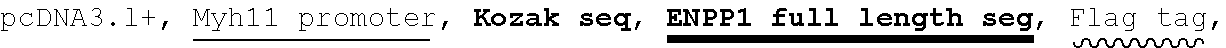

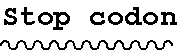

(SEQ ID NO: 84)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTCGCCGGGGAGCAGGAAGGCCACTCGGCACCATATTTAGTCAGGGGGAGCCGGCAGCCC
AGAGCTGGTATGCGGCGCTGGGAATTCCTGCAGGAAGGAGTCCGCGCCTGCCCTTTTTGGGTTGTCTCCCGCC
CGCCGCTCCCGCCGCTCCCGGGGAGGGGGACCGGCCCGGCCCGGCCCGGCCCGGGAACCTCGGAGGAGCTGGT
GCCGCGCGGGGAGCGGAGCGCCCGGGCTGCCCGCGGGTCCCCGGCCTGGCGCGGGGCCAGCCCACCGCCTCGA
CTTCCTTTTATGGCCTGTGTGTGCGTGCGTGGACAGGAGCGGGGAGGGAGGGACGGGGAGAAGACGGAGAGCC
TGGGGAAGAGAGAGAGAGAAAGCGCAGAGATAGGAGTGAGACACGCGGGAGAGATGGAGAGCAAGAGACACAG
AGACCAGAGACAAAGTGAGACAGGAGGGAGAGACAGATACATCGACAGATCTAGAGAAGCGAGAGGGACAGAG
ACAAAAGATAGAGCGAGAGACAGCAATGATCAGAGTGACAGACATGCAGAGACAGTGGCAGAGACAGAGCGAG
AGAGCCTGTGATGGAGAGAGACAGGGAATGCAATTTTAGGCGAGGAATCCTTGGGGAAGGGAAGTTGTTGAAG
GGAACTCGCAGACTCTGGGGGCACACCCACTTTCTCCTTGGATCTTGACACTTGCATCTTGTAAATAACGTAA
TTATCACCGCCACCGCCTTCCCCCATTTTGTAGCTATGGACACCAAGTCTCAGAGAAGTGAAGTGACTTGCCC
AAGGTCACGCAGCTGGCGAGTGGCGCACAGGGGAGGGGGACAGCTGAAATAATCACAGTGGGCTTATTTTTAA
TTTTTATTTGTATTTTGGTCGTGGTGATGTGGGTGGAGGTGGAGATGGCAAGTTGGGAAAAGTAAAAACTTCC
CCTTCCTGCACGGTTCCCAGCAAGGGTGGGGGCCTCCTGTCTTGCACTTTGCAAAGTTCAAGAAATCCCCTTT
CCCTACCCTTCACGCTGCACAGCCGGCCCTCTTTCCAGACAGTGCGATGCCAATAAAATGGGAAGTGGGGTGG
GAGATGTCAAGTCAGATCCACCACAGCCCCGACACGGGGAGGAAGAGGTTAAAGCCTTTGCGGCCGGAACCGA
CTCAGGGAAGACGTTCTCAAGCATCCCGCACAGACACTGCCTGCTCGACCCCCTTTCTCTAGGGATCCGGAGC
GTCTGCGACCGCCTGGGGCCGGGGCTGAGACTCCCGTCCCTGTGCGCACCTGTTCCGTGCGCCCTTGTGCGGT
GCGCACCTGTTCCGTGCACCCTTGTCCCGAGCGCCCCAGCTCCTTGCGCTCCCGCCGGGGGTGCGCCCTGCAG
GGGGCGCGGCGAGGGGGCCGCGAGGGACCCTCCCCAACTCCACCCCTTCGGCCTCCTCCCCTTTCCCAGCCGC
GGGCAGCTCCGGGTCTATAAAGAGAGGCGTCCGAGGACGCGCAGGGAGATTTGGACGCTCCGGCCTGGGAGGT
GCGTCAGATCCGAGCTCGCCATCCAGTTTCCTCTCCACTAGTCCCCCCAGTTGGAGATCTGTAAGTAGTAGTT
GTCATTCTGGGGGCAGATTGCAGGGCAGGGGGGTGTTAAAAGTCCTATAGGGTATTCTATAGGGGCTGGGGTG
CACTTAGGGGTCTCTGGCTAACTAGAGAACCCACTGCTTACTGGCTTATCGAAATTAATACGACTCACTATAG
GGAGACCCAAGCTGGCTAGCGCCACCATGGAGCGCGACGGCTGCGCGGGGGGCGGGAGCCGCGGCGGCGAGGG
CGGGCGCGCTCCCCGGGAGGGCCCGGCGGGGAACGGCCGCGATCGGGGCCGCAGCCACGCTGCCGAGGCGCCC
GGGGACCCGCAGGCCGCGGCGTCCTTGCTGGCCCCTATGGACGTGGGGGAGGAGCCGCTGGAGAAGGCGGCGC
GCGCCCGCACTGCCAAGGACCCCAACACCTATAAAGTACTCTCGCTGGTATTGTCAGTATGTGTGTTAACAAC
AATACTTGGTTGTATATTTGGGTTGAAACCAAGCTGTGCCAAAGAAGTTAAAAGTTGCAAAGGTCGCTGTTTC
GAGAGAACATTTGGGAACTGTCGCTGTGATGCTGCCTGTGTTGAGCTTGGAAACTGCTGTTTAGATTACCAGG
AGACGTGCATAGAACCAGAACATATATGGACTTGCAACAAATTCAGGTGTGGTGAGAAAAGGTTGACCAGAAG
CCTCTGTGCCTGTTCAGATGACTGCAAGGACAAGGGCGACTGCTGCATCAACTACAGTTCTGTGTGTCAAGGT
GAGAAAAGTTGGGTAGAAGAACCATGTGAGAGCATTAATGAGCCACAGTGCCCAGCAGGGTTTGAAACGCCTC
CTACCCTCTTATTTTCTTTGGATGGATTCAGGGCAGAATATTTACACACTTGGGGTGGACTTCTTCCTGTTAT
TAGCAAACTAAAAAAATGTGGAACATATACTAAAAACATGAGACCGGTATATCCAACAAAAACTTTCCCCAAT
CACTACAGCATTGTCACCGGATTGTATCCAGAATCTCATGGCATAATCGACAATAAAATGTATGATCCCAAAA
TGAATGCTTCCTTTTCACTTAAAAGTAAAGAGAAATTTAATCCTGAGTGGTACAAAGGAGAACCAATTTGGGT
CACAGCTAAGTATCAAGGCCTCAAGTCTGGCACATTTTTCTGGCCAGGATCAGATGTGGAAATTAACGGAATT
TTCCCAGACATCTATAAAATGTATAATGGTTCAGTACCATTTGAAGAAAGGATTTTAGCTGTTCTTCAGTGGC
TACAGCTTCCTAAAGATGAAAGACCACACTTTTACACTCTGTATTTAGAAGAACCAGATTCTTCAGGTCATTC
ATATGGACCAGTCAGCAGTGAAGTCATCAAAGCCTTGCAGAGGGTTGATGGTATGGTTGGTATGCTGATGGAT
GGTCTGAAAGAGCTGAACTTGCACAGATGCCTGAACCTCATCCTTATTTCAGATCATGGCATGGAACAAGGCA
GTTGTAAGAAATACATATATCTGAATAAATATTTGGGGGATGTTAAAAATATTAAAGTTATCTATGGACCTGC
AGCTCGATTGAGACCCTCTGATGTCCCAGATAAATACTATTCATTTAACTATGAAGGCATTGCCCGAAATCTT
TCTTGCCGGGAACCAAACCAGCACTTCAAACCTTACCTGAAACATTTCTTACCTAAGCGTTTGCACTTTGCTA
AGAGTGATAGAATTGAGCCCTTGACATTCTATTTGGACCCTCAGTGGCAACTTGCATTGAATCCCTCAGAAAG
GAAATATTGTGGAAGTGGATTTCATGGCTCTGACAATGTATTTTCAAATATGCAAGCCCTCTTTGTTGGCTAT
GGACCTGGATTCAAGCATGGCATTGAGGCTGACACCTTTGAAAACATTGAAGTCTATAACTTAATGTGTGATT
TACTGAATTTGACACCGGCTCCTAATAACGGAACTCATGGAAGTCTTAACCACCTTCTAAAGAATCCTGTTTA
TACGCCAAAGCATCCCAAAGAAGTGCACCCCCTGGTACAGTGCCCCTTCACAAGAAACCCCAGAGATAACCTT
GGCTGCTCATGTAACCCTTCGATTTTGCCGATTGAGGATTTTCAAACACAGTTCAATCTGACTGTGGCAGAAG
AGAAGATTATTAAGCATGAAACTTTACCCTATGGAAGACCTAGAGTTCTCCAGAAGGAAAACACCATCTGTCT
TCTTTCCCAGCACCAGTTTATGAGTGGATACAGCCAAGACATCTTAATGCCCCTTTGGACATCCTATACCGTG
GACAGAAATGACAGTTTCTCTACGGAAGACTTCTCCAACTGTCTGTACCAGGACTTTAGAATTCCTCTTAGTC
CTGTCCATAAATGTTCATTTTATAAAAATAACACCAAAGTGAGTTACGGGTTCCTCTCCCCACCACAACTAAA
TAAAAATTCAAGTGGAATATATTCTGAAGCTTTGCTTACTACAAATATAGTGCCAATGTACCAGAGTTTTCAA
GTTATATGGCGCTACTTTCATGACACCCTACTGCGAAAGTATGCTGAAGAAAGAAATGGTGTCAATGTCGTCA
GTGGTCCTGTGTTTGACTTTGATTATGATGGACGTTGTGATTCCTTAGAGAATCTGAGGCAAAAAAGAAGAGT
CATCCGTAACCAAGAAATTTTGATTCCAACTCACTTCTTTATTGTGCTAACAAGCTGTAAAGATACATCTCAG
ACGCCTTTGCACTGTGAAAACCTAGACACCTTAGCTTTCATTTTGCCTCACAGGACTGATAACAGCGAGAGCT
GTGTGCATGGGAAGCATGACTCCTCATGGGTTGAAGAATTGTTAATGTTACACAGAGCACGGATCACAGATGT
TGAGCACATCACTGGACTCAGCTTCTATCAACAAAGAAAAGAGCCAGTTTCAGACATTTTAAAGTTGAAAACA

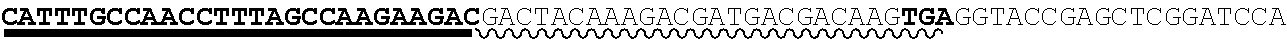

CTAGTCCAGTGTGGTGGAATTCTGCAGATATCCAGCACAGTGGCGGCCGCTCGAGTCTAGAGGGCCCGTTTAA
ACCCGCTGATCAGCCTCGACTGTGCCTTCTAGTTGCCAGCCATCTGTTGTTTGCCCCTCCCCCGTGCCTTCCT
TGACCCTGGAAGGTGCCACTCCCACTGTCCTTTCCTAATAAAATGAGGAAATTGCATCGCATTGTCTGAGTAG
GTGTCATTCTATTCTGGGGGGTGGGGTGGGGCAGGACAGCAAGGGGGAGGATTGGGAAGACAATAGCAGGCAT
GCTGGGGATGCGGTGGGCTCTATGGCTTCTGAGGCGGAAAGAACCAGCTGGGGCTCTAGGGGGTATCCCCACG
CGCCCTGTAGCGGCGCATTAAGCGCGGCGGGTGTGGTGGTTACGCGCAGCGTGACCGCTACACTTGCCAGCGC
CCTAGCGCCCGCTCCTTTCGCTTTCTTCCCTTCCTTTCTCGCCACGTTCGCCGGCTTTCCCCGTCAAGCTCTA
AATCGGGGGCTCCCTTTAGGGTTCCGATTTAGTGCTTTACGGCACCTCGACCCCAAAAAACTTGATTAGGGTG
ATGGTTCACGTAGTGGGCCATCGCCCTGATAGACGGTTTTTCGCCCTTTGACGTTGGAGTCCACGTTCTTTAA
TAGTGGACTCTTGTTCCAAACTGGAACAACACTCAACCCTATCTCGGTCTATTCTTTTGATTTATAAGGGATT
TTGCCGATTTCGGCCTATTGGTTAAAAAATGAGCTGATTTAACAAAAATTTAACGCGAATTAATTCTGTGGAA
TGTGTGTCAGTTAGGGTGTGGAAAGTCCCCAGGCTCCCCAGCAGGCAGAAGTATGCAAAGCATGCATCTCAAT
TAGTCAGCAACCAGGTGTGGAAAGTCCCCAGGCTCCCCAGCAGGCAGAAGTATGCAAAGCATGCATCTCAATT
AGTCAGCAACCATAGTCCCGCCCCTAACTCCGCCCATCCCGCCCCTAACTCCGCCCAGTTCCGCCCATTCTCC
GCCCCATGGCTGACTAATTTTTTTTATTTATGCAGAGGCCGAGGCCGCCTCTGCCTCTGAGCTATTCCAGAAG
TAGTGAGGAGGCTTTTTTGGAGGCCTAGGCTTTTGCAAAAAGCTCCCGGGAGCTTGTATATCCATTTTCGGAT
CTGATCAAGAGACAGGATGAGGATCGTTTCGCATGATTGAACAAGATGGATTGCACGCAGGTTCTCCGGCCGC
TTGGGTGGAGAGGCTATTCGGCTATGACTGGGCACAACAGACAATCGGCTGCTCTGATGCCGCCGTGTTCCGG
CTGTCAGCGCAGGGGCGCCCGGTTCTTTTTGTCAAGACCGACCTGTCCGGTGCCCTGAATGAACTGCAGGACG
AGGCAGCGCGGCTATCGTGGCTGGCCACGACGGGCGTTCCTTGCGCAGCTGTGCTCGACGTTGTCACTGAAGC
GGGAAGGGACTGGCTGCTATTGGGCGAAGTGCCGGGGCAGGATCTCCTGTCATCTCACCTTGCTCCTGCCGAG
AAAGTATCCATCATGGCTGATGCAATGCGGCGGCTGCATACGCTTGATCCGGCTACCTGCCCATTCGACCACC
AAGCGAAACATCGCATCGAGCGAGCACGTACTCGGATGGAAGCCGGTCTTGTCGATCAGGATGATCTGGACGA
AGAGCATCAGGGGCTCGCGCCAGCCGAACTGTTCGCCAGGCTCAAGGCGCGCATGCCCGACGGCGAGGATCTC
GTCGTGACCCATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGAAGAGCTTGG
CGGCGAATGGGCTGACCGCTTCCTCGTGCTTTACGGTATCGCCGCTCCCGATTCGCAGCGCATCGCCTTCTAT
CGCCTTCTTGACGAGTTCTTCTGAGCGGGACTCTGGGGTTCGAAATGACCGACCAAGCGACGCCCAACCTGCC
ATCACGAGATTTCGATTCCACCGCCGCCTTCTATGAAAGGTTGGGCTTCGGAATCGTTTTCCGGGACGCCGGC
TGGATGATCCTCCAGCGCGGGGATCTCATGCTGGAGTTCTTCGCCCACCCCAACTTGTTTATTGCAGCTTATA
ATGGTTACAAATAAAGCAATAGCATCACAAATTTCACAAATAAAGCATTTTTTTCACTGCATTCTAGTTGTGG
TTTGTCCAAACTCATCAATGTATCTTATCATGTCTGTATACCGTCGACCTCTAGCTAGAGCTTGGCGTAATCA
TGGTCATAGCTGTTTCCTGTGTGAAATTGTTATCCGCTCACAATTCCACACAACATACGAGCCGGAAGCATAA
AGTGTAAAGCCTGGGGTGCCTAATGAGTGAGCTAACTCACATTAATTGCGTTGCGCTCACTGCCCGCTTTCCA
GTCGGGAAACCTGTCGTGCCAGCTGCATTAATGAATCGGCCAACGCGCGGGGAGAGGCGGTTTGCGTATTGGG
CGCTCTTCCGCTTCCTCGCTCACTGACTCGCTGCGCTCGGTCGTTCGGCTGCGGCGAGCGGTATCAGCTCACT
CAAAGGCGGTAATACGGTTATCCACAGAATCAGGGGATAACGCAGGAAAGAACATGTGAGCAAAAGGCCAGCA
AAAGGCCAGGAACCGTAAAAAGGCCGCGTTGCTGGCGTTTTTCCATAGGCTCCGCCCCCCTGACGAGCATCAC
AAAAATCGACGCTCAAGTCAGAGGTGGCGAAACCCGACAGGACTATAAAGATACCAGGCGTTTCCCCCTGGAA
GCTCCCTCGTGCGCTCTCCTGTTCCGACCCTGCCGCTTACCGGATACCTGTCCGCCTTTCTCCCTTCGGGAAG
CGTGGCGCTTTCTCATAGCTCACGCTGTAGGTATCTCAGTTCGGTGTAGGTCGTTCGCTCCAAGCTGGGCTGT
GTGCACGAACCCCCCGTTCAGCCCGACCGCTGCGCCTTATCCGGTAACTATCGTCTTGAGTCCAACCCGGTAA
GACACGACTTATCGCCACTGGCAGCAGCCACTGGTAACAGGATTAGCAGAGCGAGGTATGTAGGCGGTGCTAC
AGAGTTCTTGAAGTGGTGGCCTAACTACGGCTACACTAGAAGAACAGTATTTGGTATCTGCGCTCTGCTGAAG
CCAGTTACCTTCGGAAAAAGAGTTGGTAGCTCTTGATCCGGCAAACAAACCACCGCTGGTAGCGGTGGTTTTT
TTGTTTGCAAGCAGCAGATTACGCGCAGAAAAAAAGGATCTCAAGAAGATCCTTTGATCTTTTCTACGGGGTC
TGACGCTCAGTGGAACGAAAACTCACGTTAAGGGATTTTGGTCATGAGATTATCAAAAAGGATCTTCACCTAG
ATCCTTTTAAATTAAAAATGAAGTTTTAAATCAATCTAAAGTATATATGAGTAAACTTGGTCTGACAGTTACC
AATGCTTAATCAGTGAGGCACCTATCTCAGCGATCTGTCTATTTCGTTCATCCATAGTTGCCTGACTCCCCGT
CGTGTAGATAACTACGATACGGGAGGGCTTACCATCTGGCCCCAGTGCTGCAATGATACCGCGAGACCCACGC
TCACCGGCTCCAGATTTATCAGCAATAAACCAGCCAGCCGGAAGGGCCGAGCGCAGAAGTGGTCCTGCAACTT
TATCCGCCTCCATCCAGTCTATTAATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGTTAGCTCCTTCGGTCCTCCGA
TCGTTGTCAGAAGTAAGTTGGCCGCAGTGTTATCACTCATGGTTATGGCAGCACTGCATAATTCTCTTACTGT
CATGCCATCCGTAAGATGCTTTTCTGTGACTGGTGAGTACTCAACCAAGTCATTCTGAGAATAGTGTATGCGG
CGACCGAGTTGCTCTTGCCCGGCGTCAATACGGGATAATACCGCGCCACATAGCAGAACTTTAAAAGTGCTCA
TCATTGGAAAACGTTCTTCGGGGCGAAAACTCTCAAGGATCTTACCGCTGTTGAGATCCAGTTCGATGTAACC
CACTCGTGCACCCAACTGATCTTCAGCATCTTTTACTTTCACCAGCGTTTCTGGGTGAGCAAAAACAGGAAGG
CAAAATGCCGCAAAAAAGGGAATAAGGGCGACACGGAAATGTTGAATACTCATACTCTTCCTTTTTCAATATT
ATTGAAGCATTTATCAGGGTTATTGTCTCATGAGCGGATACATATTTGAATGTATTTAGAAAAATAAACAAAT
AGGGGTTCCGCGCACATTTCCCCGAAAAGTGCCACCTGACGTC
MGP_Kozak_ENPP1 Full Length_FLAG_pcDNA3.1 (+)

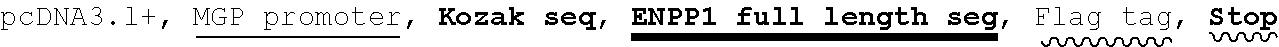

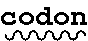

(SEQ ID NO: 85)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTAAGATTATAGTTGTCATTTGAACTTGGGGATAAAGGAGACATCTATGACTTGGCTGGA
AAAGACAGAGCTAATGTACATTGCAAAGCACATATTTATAGCAGGAAAATGGGAAGATTTCTCTTTAATTCTG
GAGATGGAGTGGGGATGGGGAGAGTAGACTACTCATTTTAAGGGTGAAACATTGGAATTCAACTTGTTTGATG
TTATATTAATTGGTGGTTAATTACTAAGCTAAGTACGTATAAAACTTTTATCTATGGCTAGCTTGTCCCCCCA
AAGTCATGCAATATAGTGAACTGGCTTTCGCACTTTAAATTATTCATTGATCATGTAATGATTCAGATGATTC
ATCTTCCAAGATGGACACTGAAACTAACACTCATAGTAGGTTGTGGTTTAAAGAGTGGAACAACCGCCAGTCT
CATTAGTGGAAATTGTGATGGTTGAATTTATCAAGGATGAACATACACGGTCTTCTTTCTGAGATTTTCTTTA
AGATTTTCGCACAGATAATCTATTTCTTAGGTTTTGGAGAGAAAACTTGAATTTTATTGATCCCTCAGAACTC
AATCTTTCAGATTTCAAAGGAGCTATTTCTTTTAATGGGGACTCTGTTAATATTTATAAAAGCTCTTCACAGG
ATGGAGGGTGGGAGGGAAACTCCATCCCAACAAGACAAAAAGAATGAAGCATGAGGCTCCACCTAGTTCATCA
CTGCTCCTTGAAATACATCAGTATTGAAAGACACATCCACCCCACCCCCAACCCAGCCCTATTGCTGTTCCAG
CTCAAGAGTCAGAGGTCCCGAAGCTGTAGCTCTTCTACAATCTGCTGCTCTGTGACTTCAAGTCTGTTGTCTG
CAAAGAAAACTATTGGGTTCCCAAGCAAGAGAGGCACATCTGGTAGGACAGATTTTGTGATTGCAAAAGAAGG
GGGAAAAAAAGAAAGAAAGAAAAGACCTCTCTATACAAGATAACCAGAGGCATCAAACTGAAATCCTCCTGTG
GAAAATAAGCTAGTACTTCTGGGCCTGATGGTGTAGTGAAAACCTGTGCTTGAGGATACATTACAGTGAAAGA
GCAAAGTGAATAGTAAGTAGCTATTACTTACCTCCTTAGGGAGGTGTGTTGTTTGTCTGTACATCCCCCACAG
CACCTAGCACAGTACCTTGCATCTCACCTGCCACTCACTAAAAAGTCTATCAAGTTAGTTAATTATCGAGACA
ACGCCCTCAGAAATGAGAGAACAGTACCCTCTTATCCTTGCTGCACTTTCCAGCACTGATACGCTGCCTAAAA
GAGGACTAGGGCACAGGTTTGAATTAATGTCACAAAACTGGATGGGCAAGTTACAACGGTGTTGATTAAGGAA
ACAGAACTCATGGTGCACCGGATATCTCCATCCTGATGAACCCTTGGAAAAATGCCAAAGATGCATATCCCCA
GGCAAATGCCTGATTAGTCTGGGATTGATAGATTGGTCTAGGATTCAGCCCTACTGGGAAGATGTCTAAATTA
TAATCAGTGTAGAAAGCGAAGTTCTCCTAGAAGAAGAGGCAAAGGTTAAAAAGAAGAAAAGAAAAGAAAGTGA
AGTCCTTTCTCCCCCAAAACCTCTCATCAATCAATCAGGGTAACAAACAGAACACTAGGGCTCTGTCTGTGGA
CCAAACCCAAAAGCCCTGCGGTCAGGGCCAGGAGGGTAGATCATGTGTTTGTGGCAACTTCCTCTGTGGGCTT
TTGCCCAGGTCTGTCCCCAAGCATACGATGGCCAAAACTTCTGCACCAGAGCAGCATCCTGTGTAACACAGTC
AGGTCCAGCAGTTAGGGAAAACTGCCCACTCAGAGTAGATAATATCTGGAAGGAATGACTGTTTGGGAAAAGT
TCCAATGCTAGTTCAGTGCCAACCCTTCCCCACCTTCTCCAGCTCTCTCCCACTGGTTCCTCCCCTCTCAACT
GCTCTGGTTCTTATAAAAACCTCACAGCCTTCCACTAACATCCCGTAGGAGCCTCTCTCCCTACTGCTGCTAC
ACAAGACCCTGAGACTGACCTGCAGGACGAAACCCTCTGGCTAACTAGAGAACCCACTGCTTACTGGCTTATC
GAAATTAATACGACTCACTATAGGGAGACCCAAGCTGGCTAGCGCCACCATGGAGCGCGACGGCTGCGCGGGG
GGCGGGAGCCGCGGCGGCGAGGGCGGGCGCGCTCCCCGGGAGGGCCCGGCGGGGAACGGCCGCGATCGGGGCC
GCAGCCACGCTGCCGAGGCGCCCGGGGACCCGCAGGCCGCGGCGTCCTTGCTGGCCCCTATGGACGTGGGGGA
GGAGCCGCTGGAGAAGGCGGCGCGCGCCCGCACTGCCAAGGACCCCAACACCTATAAAGTACTCTCGCTGGTA
TTGTCAGTATGTGTGTTAACAACAATACTTGGTTGTATATTTGGGTTGAAACCAAGCTGTGCCAAAGAAGTTA
AAAGTTGCAAAGGTCGCTGTTTCGAGAGAACATTTGGGAACTGTCGCTGTGATGCTGCCTGTGTTGAGCTTGG
AAACTGCTGTTTAGATTACCAGGAGACGTGCATAGAACCAGAACATATATGGACTTGCAACAAATTCAGGTGT
GGTGAGAAAAGGTTGACCAGAAGCCTCTGTGCCTGTTCAGATGACTGCAAGGACAAGGGCGACTGCTGCATCA
ACTACAGTTCTGTGTGTCAAGGTGAGAAAAGTTGGGTAGAAGAACCATGTGAGAGCATTAATGAGCCACAGTG
CCCAGCAGGGTTTGAAACGCCTCCTACCCTCTTATTTTCTTTGGATGGATTCAGGGCAGAATATTTACACACT
TGGGGTGGACTTCTTCCTGTTATTAGCAAACTAAAAAAATGTGGAACATATACTAAAAACATGAGACCGGTAT
ATCCAACAAAAACTTTCCCCAATCACTACAGCATTGTCACCGGATTGTATCCAGAATCTCATGGCATAATCGA
CAATAAAATGTATGATCCCAAAATGAATGCTTCCTTTTCACTTAAAAGTAAAGAGAAATTTAATCCTGAGTGG
TACAAAGGAGAACCAATTTGGGTCACAGCTAAGTATCAAGGCCTCAAGTCTGGCACATTTTTCTGGCCAGGAT
CAGATGTGGAAATTAACGGAATTTTCCCAGACATCTATAAAATGTATAATGGTTCAGTACCATTTGAAGAAAG
GATTTTAGCTGTTCTTCAGTGGCTACAGCTTCCTAAAGATGAAAGACCACACTTTTACACTCTGTATTTAGAA
GAACCAGATTCTTCAGGTCATTCATATGGACCAGTCAGCAGTGAAGTCATCAAAGCCTTGCAGAGGGTTGATG
GTATGGTTGGTATGCTGATGGATGGTCTGAAAGAGCTGAACTTGCACAGATGCCTGAACCTCATCCTTATTTC
AGATCATGGCATGGAACAAGGCAGTTGTAAGAAATACATATATCTGAATAAATATTTGGGGGATGTTAAAAAT
ATTAAAGTTATCTATGGACCTGCAGCTCGATTGAGACCCTCTGATGTCCCAGATAAATACTATTCATTTAACT
ATGAAGGCATTGCCCGAAATCTTTCTTGCCGGGAACCAAACCAGCACTTCAAACCTTACCTGAAACATTTCTT
ACCTAAGCGTTTGCACTTTGCTAAGAGTGATAGAATTGAGCCCTTGACATTCTATTTGGACCCTCAGTGGCAA
CTTGCATTGAATCCCTCAGAAAGGAAATATTGTGGAAGTGGATTTCATGGCTCTGACAATGTATTTTCAAATA
TGCAAGCCCTCTTTGTTGGCTATGGACCTGGATTCAAGCATGGCATTGAGGCTGACACCTTTGAAAACATTGA
AGTCTATAACTTAATGTGTGATTTACTGAATTTGACACCGGCTCCTAATAACGGAACTCATGGAAGTCTTAAC
CACCTTCTAAAGAATCCTGTTTATACGCCAAAGCATCCCAAAGAAGTGCACCCCCTGGTACAGTGCCCCTTCA
CAAGAAACCCCAGAGATAACCTTGGCTGCTCATGTAACCCTTCGATTTTGCCGATTGAGGATTTTCAAACACA
GTTCAATCTGACTGTGGCAGAAGAGAAGATTATTAAGCATGAAACTTTACCCTATGGAAGACCTAGAGTTCTC
CAGAAGGAAAACACCATCTGTCTTCTTTCCCAGCACCAGTTTATGAGTGGATACAGCCAAGACATCTTAATGC
CCCTTTGGACATCCTATACCGTGGACAGAAATGACAGTTTCTCTACGGAAGACTTCTCCAACTGTCTGTACCA
GGACTTTAGAATTCCTCTTAGTCCTGTCCATAAATGTTCATTTTATAAAAATAACACCAAAGTGAGTTACGGG
TTCCTCTCCCCACCACAACTAAATAAAAATTCAAGTGGAATATATTCTGAAGCTTTGCTTACTACAAATATAG
TGCCAATGTACCAGAGTTTTCAAGTTATATGGCGCTACTTTCATGACACCCTACTGCGAAAGTATGCTGAAGA
AAGAAATGGTGTCAATGTCGTCAGTGGTCCTGTGTTTGACTTTGATTATGATGGACGTTGTGATTCCTTAGAG
AATCTGAGGCAAAAAAGAAGAGTCATCCGTAACCAAGAAATTTTGATTCCAACTCACTTCTTTATTGTGCTAA
CAAGCTGTAAAGATACATCTCAGACGCCTTTGCACTGTGAAAACCTAGACACCTTAGCTTTCATTTTGCCTCA
CAGGACTGATAACAGCGAGAGCTGTGTGCATGGGAAGCATGACTCCTCATGGGTTGAAGAATTGTTAATGTTA
CACAGAGCACGGATCACAGATGTTGAGCACATCACTGGACTCAGCTTCTATCAACAAAGAAAAGAGCCAGTTT

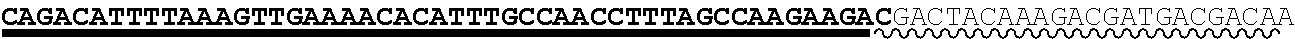

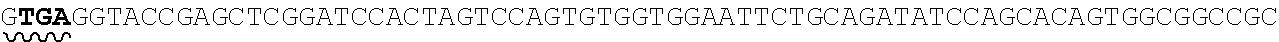

TCGAGTCTAGAGGGCCCGTTTAAACCCGCTGATCAGCCTCGACTGTGCCTTCTAGTTGCCAGCCATCTGTTGT
TTGCCCCTCCCCCGTGCCTTCCTTGACCCTGGAAGGTGCCACTCCCACTGTCCTTTCCTAATAAAATGAGGAA
ATTGCATCGCATTGTCTGAGTAGGTGTCATTCTATTCTGGGGGGTGGGGTGGGGCAGGACAGCAAGGGGGAGG
ATTGGGAAGACAATAGCAGGCATGCTGGGGATGCGGTGGGCTCTATGGCTTCTGAGGCGGAAAGAACCAGCTG
GGGCTCTAGGGGGTATCCCCACGCGCCCTGTAGCGGCGCATTAAGCGCGGCGGGTGTGGTGGTTACGCGCAGC
GTGACCGCTACACTTGCCAGCGCCCTAGCGCCCGCTCCTTTCGCTTTCTTCCCTTCCTTTCTCGCCACGTTCG
CCGGCTTTCCCCGTCAAGCTCTAAATCGGGGGCTCCCTTTAGGGTTCCGATTTAGTGCTTTACGGCACCTCGA
CCCCAAAAAACTTGATTAGGGTGATGGTTCACGTAGTGGGCCATCGCCCTGATAGACGGTTTTTCGCCCTTTG
ACGTTGGAGTCCACGTTCTTTAATAGTGGACTCTTGTTCCAAACTGGAACAACACTCAACCCTATCTCGGTCT
ATTCTTTTGATTTATAAGGGATTTTGCCGATTTCGGCCTATTGGTTAAAAAATGAGCTGATTTAACAAAAATT
TAACGCGAATTAATTCTGTGGAATGTGTGTCAGTTAGGGTGTGGAAAGTCCCCAGGCTCCCCAGCAGGCAGAA
GTATGCAAAGCATGCATCTCAATTAGTCAGCAACCAGGTGTGGAAAGTCCCCAGGCTCCCCAGCAGGCAGAAG
TATGCAAAGCATGCATCTCAATTAGTCAGCAACCATAGTCCCGCCCCTAACTCCGCCCATCCCGCCCCTAACT
CCGCCCAGTTCCGCCCATTCTCCGCCCCATGGCTGACTAATTTTTTTTATTTATGCAGAGGCCGAGGCCGCCT
CTGCCTCTGAGCTATTCCAGAAGTAGTGAGGAGGCTTTTTTGGAGGCCTAGGCTTTTGCAAAAAGCTCCCGGG
AGCTTGTATATCCATTTTCGGATCTGATCAAGAGACAGGATGAGGATCGTTTCGCATGATTGAACAAGATGGA
TTGCACGCAGGTTCTCCGGCCGCTTGGGTGGAGAGGCTATTCGGCTATGACTGGGCACAACAGACAATCGGCT
GCTCTGATGCCGCCGTGTTCCGGCTGTCAGCGCAGGGGCGCCCGGTTCTTTTTGTCAAGACCGACCTGTCCGG
TGCCCTGAATGAACTGCAGGACGAGGCAGCGCGGCTATCGTGGCTGGCCACGACGGGCGTTCCTTGCGCAGCT
GTGCTCGACGTTGTCACTGAAGCGGGAAGGGACTGGCTGCTATTGGGCGAAGTGCCGGGGCAGGATCTCCTGT
CATCTCACCTTGCTCCTGCCGAGAAAGTATCCATCATGGCTGATGCAATGCGGCGGCTGCATACGCTTGATCC
GGCTACCTGCCCATTCGACCACCAAGCGAAACATCGCATCGAGCGAGCACGTACTCGGATGGAAGCCGGTCTT
GTCGATCAGGATGATCTGGACGAAGAGCATCAGGGGCTCGCGCCAGCCGAACTGTTCGCCAGGCTCAAGGCGC
GCATGCCCGACGGCGAGGATCTCGTCGTGACCCATGGCGATGCCTGCTTGCCGAATATCATGGTGGAAAATGG
CCGCTTTTCTGGATTCATCGACTGTGGCCGGCTGGGTGTGGCGGACCGCTATCAGGACATAGCGTTGGCTACC
CGTGATATTGCTGAAGAGCTTGGCGGCGAATGGGCTGACCGCTTCCTCGTGCTTTACGGTATCGCCGCTCCCG
ATTCGCAGCGCATCGCCTTCTATCGCCTTCTTGACGAGTTCTTCTGAGCGGGACTCTGGGGTTCGAAATGACC
GACCAAGCGACGCCCAACCTGCCATCACGAGATTTCGATTCCACCGCCGCCTTCTATGAAAGGTTGGGCTTCG
GAATCGTTTTCCGGGACGCCGGCTGGATGATCCTCCAGCGCGGGGATCTCATGCTGGAGTTCTTCGCCCACCC
CAACTTGTTTATTGCAGCTTATAATGGTTACAAATAAAGCAATAGCATCACAAATTTCACAAATAAAGCATTT
TTTTCACTGCATTCTAGTTGTGGTTTGTCCAAACTCATCAATGTATCTTATCATGTCTGTATACCGTCGACCT
CTAGCTAGAGCTTGGCGTAATCATGGTCATAGCTGTTTCCTGTGTGAAATTGTTATCCGCTCACAATTCCACA
CAACATACGAGCCGGAAGCATAAAGTGTAAAGCCTGGGGTGCCTAATGAGTGAGCTAACTCACATTAATTGCG
TTGCGCTCACTGCCCGCTTTCCAGTCGGGAAACCTGTCGTGCCAGCTGCATTAATGAATCGGCCAACGCGCGG
GGAGAGGCGGTTTGCGTATTGGGCGCTCTTCCGCTTCCTCGCTCACTGACTCGCTGCGCTCGGTCGTTCGGCT
GCGGCGAGCGGTATCAGCTCACTCAAAGGCGGTAATACGGTTATCCACAGAATCAGGGGATAACGCAGGAAAG
AACATGTGAGCAAAAGGCCAGCAAAAGGCCAGGAACCGTAAAAAGGCCGCGTTGCTGGCGTTTTTCCATAGGC
TCCGCCCCCCTGACGAGCATCACAAAAATCGACGCTCAAGTCAGAGGTGGCGAAACCCGACAGGACTATAAAG
ATACCAGGCGTTTCCCCCTGGAAGCTCCCTCGTGCGCTCTCCTGTTCCGACCCTGCCGCTTACCGGATACCTG
TCCGCCTTTCTCCCTTCGGGAAGCGTGGCGCTTTCTCATAGCTCACGCTGTAGGTATCTCAGTTCGGTGTAGG
TCGTTCGCTCCAAGCTGGGCTGTGTGCACGAACCCCCCGTTCAGCCCGACCGCTGCGCCTTATCCGGTAACTA
TCGTCTTGAGTCCAACCCGGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCTACACTAGAAGAACAGTAT
TTGGTATCTGCGCTCTGCTGAAGCCAGTTACCTTCGGAAAAAGAGTTGGTAGCTCTTGATCCGGCAAACAAAC
CACCGCTGGTAGCGGTGGTTTTTTTGTTTGCAAGCAGCAGATTACGCGCAGAAAAAAAGGATCTCAAGAAGAT
CCTTTGATCTTTTCTACGGGGTCTGACGCTCAGTGGAACGAAAACTCACGTTAAGGGATTTTGGTCATGAGAT
TATCAAAAAGGATCTTCACCTAGATCCTTTTAAATTAAAAATGAAGTTTTAAATCAATCTAAAGTATATATGA
GTAAACTTGGTCTGACAGTTACCAATGCTTAATCAGTGAGGCACCTATCTCAGCGATCTGTCTATTTCGTTCA
TCCATAGTTGCCTGACTCCCCGTCGTGTAGATAACTACGATACGGGAGGGCTTACCATCTGGCCCCAGTGCTG
CAATGATACCGCGAGACCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGGGAAGCTAGAGTAAGT
AGTTCGCCAGTTAATAGTTTGCGCAACGTTGTTGCCATTGCTACAGGCATCGTGGTGTCACGCTCGTCGTTTG
GTATGGCTTCATTCAGCTCCGGTTCCCAACGATCAAGGCGAGTTACATGATCCCCCATGTTGTGCAAAAAAGC
GGTTAGCTCCTTCGGTCCTCCGATCGTTGTCAGAAGTAAGTTGGCCGCAGTGTTATCACTCATGGTTATGGCA
GCACTGCATAATTCTCTTACTGTCATGCCATCCGTAAGATGCTTTTCTGTGACTGGTGAGTACTCAACCAAGT
CATTCTGAGAATAGTGTATGCGGCGACCGAGTTGCTCTTGCCCGGCGTCAATACGGGATAATACCGCGCCACA
TAGCAGAACTTTAAAAGTGCTCATCATTGGAAAACGTTCTTCGGGGCGAAAACTCTCAAGGATCTTACCGCTG
TTGAGATCCAGTTCGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACACGGAAATGTTGAATACT
CATACTCTTCCTTTTTCAATATTATTGAAGCATTTATCAGGGTTATTGTCTCATGAGCGGATACATATTTGAA
TGTATTTAGAAAAATAAACAAATAGGGGTTCCGCGCACATTTCCCCGAAAAGTGCCACCTGACGTC
MiniH9Prom_Kozak_ENPP1 Full Length_FLAG_pcDNA3.1 (+)
(SEQ ID NO: 86)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTGAAAACTACAATTGACTATCTCTAAATTTTAAAATGTGATGATAGTTACTACTTCAGT
GGTCCACACTGTTTCTATCCTTTGGAACATAAGATTTTGTTGTGTAGCTTAAGGAAGAGGAATTCAGCAGTTG
ACTATCTCGATTGTTCTGGGAATTTTGGAGGCTGAGGGGAAATTTTCACATATAAGCGCTGGGTTTGCTGGGG
TATTGAGAGTGACCTCTGGCTAACTAGAGAACCCACTGCTTACTGGCTTATCGAAATTAATACGACTCACTAT
AGGGAGACCCAAGCTGGCTAGCGCCACCATGGAGCGCGACGGCTGCGCGGGGGGCGGGAGCCGCGGCGGCGAG
GGCGGGCGCGCTCCCCGGGAGGGCCCGGCGGGGAACGGCCGCGATCGGGGCCGCAGCCACGCTGCCGAGGCGC
CCGGGGACCCGCAGGCCGCGGCGTCCTTGCTGGCCCCTATGGACGTGGGGGAGGAGCCGCTGGAGAAGGCGGC
GCGCGCCCGCACTGCCAAGGACCCCAACACCTATAAAGTACTCTCGCTGGTATTGTCAGTATGTGTGTTAACA
ACAATACTTGGTTGTATATTTGGGTTGAAACCAAGCTGTGCCAAAGAAGTTAAAAGTTGCAAAGGTCGCTGTT
TCGAGAGAACATTTGGGAACTGTCGCTGTGATGCTGCCTGTGTTGAGCTTGGAAACTGCTGTTTAGATTACCA
GGAGACGTGCATAGAACCAGAACATATATGGACTTGCAACAAATTCAGGTGTGGTGAGAAAAGGTTGACCAGA
AGCCTCTGTGCCTGTTCAGATGACTGCAAGGACAAGGGCGACTGCTGCATCAACTACAGTTCTGTGTGTCAAG
GTGAGAAAAGTTGGGTAGAAGAACCATGTGAGAGCATTAATGAGCCACAGTGCCCAGCAGGGTTTGAAACGCC
TCCTACCCTCTTATTTTCTTTGGATGGATTCAGGGCAGAATATTTACACACTTGGGGTGGACTTCTTCCTGTT
ATTAGCAAACTAAAAAAATGTGGAACATATACTAAAAACATGAGACCGGTATATCCAACAAAAACTTTCCCCA
ATCACTACAGCATTGTCACCGGATTGTATCCAGAATCTCATGGCATAATCGACAATAAAATGTATGATCCCAA
AATGAATGCTTCCTTTTCACTTAAAAGTAAAGAGAAATTTAATCCTGAGTGGTACAAAGGAGAACCAATTTGG
GTCACAGCTAAGTATCAAGGCCTCAAGTCTGGCACATTTTTCTGGCCAGGATCAGATGTGGAAATTAACGGAA
TTTTCCCAGACATCTATAAAATGTATAATGGTTCAGTACCATTTGAAGAAAGGATTTTAGCTGTTCTTCAGTG
GCTACAGCTTCCTAAAGATGAAAGACCACACTTTTACACTCTGTATTTAGAAGAACCAGATTCTTCAGGTCAT
TCATATGGACCAGTCAGCAGTGAAGTCATCAAAGCCTTGCAGAGGGTTGATGGTATGGTTGGTATGCTGATGG
ATGGTCTGAAAGAGCTGAACTTGCACAGATGCCTGAACCTCATCCTTATTTCAGATCATGGCATGGAACAAGG
CAGTTGTAAGAAATACATATATCTGAATAAATATTTGGGGGATGTTAAAAATATTAAAGTTATCTATGGACCT
GCAGCTCGATTGAGACCCTCTGATGTCCCAGATAAATACTATTCATTTAACTATGAAGGCATTGCCCGAAATC
TTTCTTGCCGGGAACCAAACCAGCACTTCAAACCTTACCTGAAACATTTCTTACCTAAGCGTTTGCACTTTGC
TAAGAGTGATAGAATTGAGCCCTTGACATTCTATTTGGACCCTCAGTGGCAACTTGCATTGAATCCCTCAGAA
AGGAAATATTGTGGAAGTGGATTTCATGGCTCTGACAATGTATTTTCAAATATGCAAGCCCTCTTTGTTGGCT
ATGGACCTGGATTCAAGCATGGCATTGAGGCTGACACCTTTGAAAACATTGAAGTCTATAACTTAATGTGTGA
TTTACTGAATTTGACACCGGCTCCTAATAACGGAACTCATGGAAGTCTTAACCACCTTCTAAAGAATCCTGTT
TATACGCCAAAGCATCCCAAAGAAGTGCACCCCCTGGTACAGTGCCCCTTCACAAGAAACCCCAGAGATAACC
TTGGCTGCTCATGTAACCCTTCGATTTTGCCGATTGAGGATTTTCAAACACAGTTCAATCTGACTGTGGCAGA
AGAGAAGATTATTAAGCATGAAACTTTACCCTATGGAAGACCTAGAGTTCTCCAGAAGGAAAACACCATCTGT
CTTCTTTCCCAGCACCAGTTTATGAGTGGATACAGCCAAGACATCTTAATGCCCCTTTGGACATCCTATACCG
TGGACAGAAATGACAGTTTCTCTACGGAAGACTTCTCCAACTGTCTGTACCAGGACTTTAGAATTCCTCTTAG
TCCTGTCCATAAATGTTCATTTTATAAAAATAACACCAAAGTGAGTTACGGGTTCCTCTCCCCACCACAACTA
AATAAAAATTCAAGTGGAATATATTCTGAAGCTTTGCTTACTACAAATATAGTGCCAATGTACCAGAGTTTTC
AAGTTATATGGCGCTACTTTCATGACACCCTACTGCGAAAGTATGCTGAAGAAAGAAATGGTGTCAATGTCGT
CAGTGGTCCTGTGTTTGACTTTGATTATGATGGACGTTGTGATTCCTTAGAGAATCTGAGGCAAAAAAGAAGA
GTCATCCGTAACCAAGAAATTTTGATTCCAACTCACTTCTTTATTGTGCTAACAAGCTGTAAAGATACATCTC
AGACGCCTTTGCACTGTGAAAACCTAGACACCTTAGCTTTCATTTTGCCTCACAGGACTGATAACAGCGAGAG
CTGTGTGCATGGGAAGCATGACTCCTCATGGGTTGAAGAATTGTTAATGTTACACAGAGCACGGATCACAGAT
GTTGAGCACATCACTGGACTCAGCTTCTATCAACAAAGAAAAGAGCCAGTTTCAGACATTTTAAAGTTGAAAA
CACATTTGCCAACCTTTAGCCAAGAAGACGACTACAAAGACGATGACGACAAGTGAGGTACCGAGCTCGGATC
CACTAGTCCAGTGTGGTGGAATTCTGCAGATATCCAGCACAGTGGCGGCCGCTCGAGTCTAGAGGGCCCGTTT
AAACCCGCTGATCAGCCTCGACTGTGCCTTCTAGTTGCCAGCCATCTGTTGTTTGCCCCTCCCCCGTGCCTTC
CTTGACCCTGGAAGGTGCCACTCCCACTGTCCTTTCCTAATAAAATGAGGAAATTGCATCGCATTGTCTGAGT
AGGTGTCATTCTATTCTGGGGGGTGGGGTGGGGCAGGACAGCAAGGGGGAGGATTGGGAAGACAATAGCAGGC
ATGCTGGGGATGCGGTGGGCTCTATGGCTTCTGAGGCGGAAAGAACCAGCTGGGGCTCTAGGGGGTATCCCCA
CGCGCCCTGTAGCGGCGCATTAAGCGCGGCGGGTGTGGTGGTTACGCGCAGCGTGACCGCTACACTTGCCAGC
GCCCTAGCGCCCGCTCCTTTCGCTTTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCTCGACCCCAAAAAACTTGATTAGGG
TGATGGTTCACGTAGTGGGCCATCGCCCTGATAGACGGTTTTTCGCCCTTTGACGTTGGAGTCCACGTTCTTT
AATAGTGGACTCTTGTTCCAAACTGGAACAACACTCAACCCTATCTCGGTCTATTCTTTTGATTTATAAGGGA
TTTTGCCGATTTCGGCCTATTGGTTAAAAAATGAGCTGATTTAACAAAAATTTAACGCGAATTAATTCTGTGG
AATGTGTGTCAGTTAGGGTGTGGAAAGTCCCCAGGCTCCCCAGCAGGCAGAAGTATGCAAAGCATGCATCTCA
ATTAGTCAGCAACCAGGTGTGGAAAGTCCCCAGGCTCCCCAGCAGGCAGAAGTATGCAAAGCATGCATCTCAA
TTAGTCAGCAACCATAGTCCCGCCCCTAACTCCGCCCATCCCGCCCCTAACTCCGCCCAGTTCCGCCCATTCT
CCGCCCCATGGCTGACTAATTTTTTTTATTTATGCAGAGGCCGAGGCCGCCTCTGCCTCTGAGCTATTCCAGA
AGTAGTGAGGAGGCTTTTTTGGAGGCCTAGGCTTTTGCAAAAAGCTCCCGGGAGCTTGTATATCCATTTTCGG
ATCTGATCAAGAGACAGGATGAGGATCGTTTCGCATGATTGAACAAGATGGATTGCACGCAGGTTCTCCGGCC
GCTTGGGTGGAGAGGCTATTCGGCTATGACTGGGCACAACAGACAATCGGCTGCTCTGATGCCGCCGTGTTCC
GGCTGTCAGCGCAGGGGCGCCCGGTTCTTTTTGTCAAGACCGACCTGTCCGGTGCCCTGAATGAACTGCAGGA
CGAGGCAGCGCGGCTATCGTGGCTGGCCACGACGGGCGTTCCTTGCGCAGCTGTGCTCGACGTTGTCACTGAA
GCGGGAAGGGACTGGCTGCTATTGGGCGAAGTGCCGGGGCAGGATCTCCTGTCATCTCACCTTGCTCCTGCCG
AGAAAGTATCCATCATGGCTGATGCAATGCGGCGGCTGCATACGCTTGATCCGGCTACCTGCCCATTCGACCA
CCAAGCGAAACATCGCATCGAGCGAGCACGTACTCGGATGGAAGCCGGTCTTGTCGATCAGGATGATCTGGAC
GAAGAGCATCAGGGGCTCGCGCCAGCCGAACTGTTCGCCAGGCTCAAGGCGCGCATGCCCGACGGCGAGGATC
TCGTCGTGACCCATGGCGATGCCTGCTTGCCGAATATCATGGTGGAAAATGGCCGCTTTTCTGGATTCATCGA
CTGTGGCCGGCTGGGTGTGGCGGACCGCTATCAGGACATAGCGTTGGCTACCCGTGATATTGCTGAAGAGCTT
GGCGGCGAATGGGCTGACCGCTTCCTCGTGCTTTACGGTATCGCCGCTCCCGATTCGCAGCGCATCGCCTTCT
ATCGCCTTCTTGACGAGTTCTTCTGAGCGGGACTCTGGGGTTCGAAATGACCGACCAAGCGACGCCCAACCTG
CCATCACGAGATTTCGATTCCACCGCCGCCTTCTATGAAAGGTTGGGCTTCGGAATCGTTTTCCGGGACGCCG
GCTGGATGATCCTCCAGCGCGGGGATCTCATGCTGGAGTTCTTCGCCCACCCCAACTTGTTTATTGCAGCTTA
TAATGGTTACAAATAAAGCAATAGCATCACAAATTTCACAAATAAAGCATTTTTTTCACTGCATTCTAGTTGT
GGTTTGTCCAAACTCATCAATGTATCTTATCATGTCTGTATACCGTCGACCTCTAGCTAGAGCTTGGCGTAAT
CATGGTCATAGCTGTTTCCTGTGTGAAATTGTTATCCGCTCACAATTCCACACAACATACGAGCCGGAAGCAT
AAAGTGTAAAGCCTGGGGTGCCTAATGAGTGAGCTAACTCACATTAATTGCGTTGCGCTCACTGCCCGCTTTC
CAGTCGGGAAACCTGTCGTGCCAGCTGCATTAATGAATCGGCCAACGCGCGGGGAGAGGCGGTTTGCGTATTG
GGCGCTCTTCCGCTTCCTCGCTCACTGACTCGCTGCGCTCGGTCGTTCGGCTGCGGCGAGCGGTATCAGCTCA
CTCAAAGGCGGTAATACGGTTATCCACAGAATCAGGGGATAACGCAGGAAAGAACATGTGAGCAAAAGGCCAG
CAAAAGGCCAGGAACCGTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATACCAGGCGTTTCCCCCTGG
AAGCTCCCTCGTGCGCTCTCCTGTTCCGACCCTGCCGCTTACCGGATACCTGTCCGCCTTTCTCCCTTCGGGA
AGCGTGGCGCTTTCTCATAGCTCACGCTGTAGGTATCTCAGTTCGGTGTAGGTCGTTCGCTCCAAGCTGGGCT
GTGTGCACGAACCCCCCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAGGTATGTAGGCGGTGCT
ACAGAGTTCTTGAAGTGGTGGCCTAACTACGGCTACACTAGAAGAACAGTATTTGGTATCTGCGCTCTGCTGA
AGCCAGTTACCTTCGGAAAAAGAGTTGGTAGCTCTTGATCCGGCAAACAAACCACCGCTGGTAGCGGTGGTTT
TTTTGTTTGCAAGCAGCAGATTACGCGCAGAAAAAAAGGATCTCAAGAAGATCCTTTGATCTTTTCTACGGGG
TCTGACGCTCAGTGGAACGAAAACTCACGTTAAGGGATTTTGGTCATGAGATTATCAAAAAGGATCTTCACCT
AGATCCTTTTAAATTAAAAATGAAGTTTTAAATCAATCTAAAGTATATATGAGTAAACTTGGTCTGACAGTTA
CCAATGCTTAATCAGTGAGGCACCTATCTCAGCGATCTGTCTATTTCGTTCATCCATAGTTGCCTGACTCCCC
GTCGTGTAGATAACTACGATACGGGAGGGCTTACCATCTGGCCCCAGTGCTGCAATGATACCGCGAGACCCAC
GCTCACCGGCTCCAGATTTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTCGCCAGTTAATAGTTTG
CGCAACGTTGTTGCCATTGCTACAGGCATCGTGGTGTCACGCTCGTCGTTTGGTATGGCTTCATTCAGCTCCG
GTTCCCAACGATCAAGGCGAGTTACATGATCCCCCATGTTGTGCAAAAAAGCGGTTAGCTCCTTCGGTCCTCC
GATCGTTGTCAGAAGTAAGTTGGCCGCAGTGTTATCACTCATGGTTATGGCAGCACTGCATAATTCTCTTACT
GTCATGCCATCCGTAAGATGCTTTTCTGTGACTGGTGAGTACTCAACCAAGTCATTCTGAGAATAGTGTATGC
GGCGACCGAGTTGCTCTTGCCCGGCGTCAATACGGGATAATACCGCGCCACATAGCAGAACTTTAAAAGTGCT
CATCATTGGAAAACGTTCTTCGGGGCGAAAACTCTCAAGGATCTTACCGCTGTTGAGATCCAGTTCGATGTAA
CCCACTCGTGCACCCAACTGATCTTCAGCATCTTTTACTTTCACCAGCGTTTCTGGGTGAGCAAAAACAGGAA
GGCAAAATGCCGCAAAAAAGGGAATAAGGGCGACACGGAAATGTTGAATACTCATACTCTTCCTTTTTCAATA
TTATTGAAGCATTTATCAGGGTTATTGTCTCATGAGCGGATACATATTTGAATGTATTTAGAAAAATAAACAA
ATAGGGGTTCCGCGCACATTTCCCCGAAAAGTGCCACCTGACGTC)
HDAC9 Prom2 (1350bp)_Kozak_ENPP1 Full Length_FLAG_pcDNA3.1 (+)

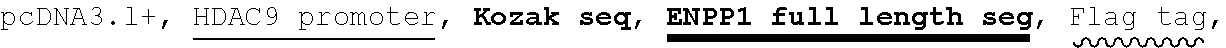

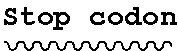

(SEQ ID NO: 87)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTATAAATGTTTTGTAGAATAAAAAAAAAAAAAGTTCTTCAAAAGAAATCTCAAATCTCC
AAATGGAAACAGGTAAAAGTGGAGCTCCCCTGGTTCCACGGAGAACCTTTTTTGAGGAAACTTAGGCAACTCG
CAGGTACCTTATGTCATGAGACAGAGTTTGAAAACTACAATTGACTATCTCTAAATTTCCTCCCAGGTCTAAA
ATGTGATGATAGTTACTACTTCAGTACATCATCCTTAAGGAAAATTATTAGGTCCACACTGTTTCTATCCTTT
GAATTTTACACATAAATTTTGTAATCAAAAGTTTATTTGTAATATCAGATGGAATCAGATAATTGCTTTTTGT
TTTTTCCACTGACAGGAACATAAGATTTTGTTGTGTAGCTTAAGTCAAACGCAGTTTGGAATATATATTTTTT
AAAAATTGTAACTTACATATCCAAATACAATTTTTCAAGAAGTAGAGTATTCAGTAGAAATTAATCTGTGAAA
GAAGAGGAATTCAGCAGTGGCCTATTTGATGAATGATTTAACGTGCTTATTTCTTCCCTTTCATCAAAACTCT
GTGTCCCCTTGTTTGCCCCCTCTGACTTCATACTCTGGAGTTGACCAAGATCCCTCTTCCATCGGATTGTTCT
GGGAATTTTGAAATAATCTGCTTTTTCCTCTCTTTCCCCTGTTGCTTCTGATGCCTTAGAATTACATTTTCCT
CGCTGATTTAGTTTAGAAAAGAGAAAAGAGCTTCCATGACTAGTAGATTATCACTTTTGGGTTTGCTCTTGGA
AGTGACAAGATGCTAGGATCCCTCTTTGGAATGTAAAATTTATCTCTTATATAGAAAGGATATAAATGTAGCA
CCAGAGACTATAAAACTCTGATACTATCTACTGTACTGTATAGCTGAACGCCACAATGTGTCTGGTAATCTAT
TGACTATCATAAATGCTATTTCTACAGAAAAGTTAGGAGGTCCATATTTCGGGCAACCAATGTATAGCTGAAT
GCAGAACAGTCATAGTTGGGTACTAACCATATATATGATTTATCCATCAACAGGTGCATATGCTCAGAAATTC
TGTATCCATAAGAAATCAGACTACTTTCTTTTCCTTTTGCAAGTAAATTGAATTTAGCCTGAGAGGCTGAGGG
GAAATTTTCACATATAAGCCACGGTTTTGTGTTTTGTGTTTTGTTTTGTTTATAGATATAGTACTAACTGGAT
GGATGCGATAAAATTCATAGGTGGTACTAAGATACAATAGGATTTGTGAAATGGACAATTGTCTTGCATAAAT
AGCAAGTAAAAAATCAAGCCTGTCCTTCATAAAAATTTTATTCTGGGGTGTCTCTGGCTAACTAGAGAACCCA
CTGCTTACTGGCTTATCGAAATTAATACGACTCACTATAGGGAGACCCAAGCTGGCTAGCGCCACCATGGAGC
GCGACGGCTGCGCGGGGGGCGGGAGCCGCGGCGGCGAGGGCGGGCGCGCTCCCCGGGAGGGCCCGGCGGGGAA
CGGCCGCGATCGGGGCCGCAGCCACGCTGCCGAGGCGCCCGGGGACCCGCAGGCCGCGGCGTCCTTGCTGGCC
CCTATGGACGTGGGGGAGGAGCCGCTGGAGAAGGCGGCGCGCGCCCGCACTGCCAAGGACCCCAACACCTATA
AAGTACTCTCGCTGGTATTGTCAGTATGTGTGTTAACAACAATACTTGGTTGTATATTTGGGTTGAAACCAAG
CTGTGCCAAAGAAGTTAAAAGTTGCAAAGGTCGCTGTTTCGAGAGAACATTTGGGAACTGTCGCTGTGATGCT
GCCTGTGTTGAGCTTGGAAACTGCTGTTTAGATTACCAGGAGACGTGCATAGAACCAGAACATATATGGACTT
GCAACAAATTCAGGTGTGGTGAGAAAAGGTTGACCAGAAGCCTCTGTGCCTGTTCAGATGACTGCAAGGACAA
GGGCGACTGCTGCATCAACTACAGTTCTGTGTGTCAAGGTGAGAAAAGTTGGGTAGAAGAACCATGTGAGAGC
ATTAATGAGCCACAGTGCCCAGCAGGGTTTGAAACGCCTCCTACCCTCTTATTTTCTTTGGATGGATTCAGGG
CAGAATATTTACACACTTGGGGTGGACTTCTTCCTGTTATTAGCAAACTAAAAAAATGTGGAACATATACTAA
AAACATGAGACCGGTATATCCAACAAAAACTTTCCCCAATCACTACAGCATTGTCACCGGATTGTATCCAGAA
TCTCATGGCATAATCGACAATAAAATGTATGATCCCAAAATGAATGCTTCCTTTTCACTTAAAAGTAAAGAGA
AATTTAATCCTGAGTGGTACAAAGGAGAACCAATTTGGGTCACAGCTAAGTATCAAGGCCTCAAGTCTGGCAC
ATTTTTCTGGCCAGGATCAGATGTGGAAATTAACGGAATTTTCCCAGACATCTATAAAATGTATAATGGTTCA
GTACCATTTGAAGAAAGGATTTTAGCTGTTCTTCAGTGGCTACAGCTTCCTAAAGATGAAAGACCACACTTTT
ACACTCTGTATTTAGAAGAACCAGATTCTTCAGGTCATTCATATGGACCAGTCAGCAGTGAAGTCATCAAAGC
CTTGCAGAGGGTTGATGGTATGGTTGGTATGCTGATGGATGGTCTGAAAGAGCTGAACTTGCACAGATGCCTG
AACCTCATCCTTATTTCAGATCATGGCATGGAACAAGGCAGTTGTAAGAAATACATATATCTGAATAAATATT
TGGGGGATGTTAAAAATATTAAAGTTATCTATGGACCTGCAGCTCGATTGAGACCCTCTGATGTCCCAGATAA
ATACTATTCATTTAACTATGAAGGCATTGCCCGAAATCTTTCTTGCCGGGAACCAAACCAGCACTTCAAACCT
TACCTGAAACATTTCTTACCTAAGCGTTTGCACTTTGCTAAGAGTGATAGAATTGAGCCCTTGACATTCTATT
TGGACCCTCAGTGGCAACTTGCATTGAATCCCTCAGAAAGGAAATATTGTGGAAGTGGATTTCATGGCTCTGA
CAATGTATTTTCAAATATGCAAGCCCTCTTTGTTGGCTATGGACCTGGATTCAAGCATGGCATTGAGGCTGAC
ACCTTTGAAAACATTGAAGTCTATAACTTAATGTGTGATTTACTGAATTTGACACCGGCTCCTAATAACGGAA
CTCATGGAAGTCTTAACCACCTTCTAAAGAATCCTGTTTATACGCCAAAGCATCCCAAAGAAGTGCACCCCCT
GGTACAGTGCCCCTTCACAAGAAACCCCAGAGATAACCTTGGCTGCTCATGTAACCCTTCGATTTTGCCGATT
GAGGATTTTCAAACACAGTTCAATCTGACTGTGGCAGAAGAGAAGATTATTAAGCATGAAACTTTACCCTATG
GAAGACCTAGAGTTCTCCAGAAGGAAAACACCATCTGTCTTCTTTCCCAGCACCAGTTTATGAGTGGATACAG
CCAAGACATCTTAATGCCCCTTTGGACATCCTATACCGTGGACAGAAATGACAGTTTCTCTACGGAAGACTTC
TCCAACTGTCTGTACCAGGACTTTAGAATTCCTCTTAGTCCTGTCCATAAATGTTCATTTTATAAAAATAACA
CCAAAGTGAGTTACGGGTTCCTCTCCCCACCACAACTAAATAAAAATTCAAGTGGAATATATTCTGAAGCTTT
GCTTACTACAAATATAGTGCCAATGTACCAGAGTTTTCAAGTTATATGGCGCTACTTTCATGACACCCTACTG
CGAAAGTATGCTGAAGAAAGAAATGGTGTCAATGTCGTCAGTGGTCCTGTGTTTGACTTTGATTATGATGGAC
GTTGTGATTCCTTAGAGAATCTGAGGCAAAAAAGAAGAGTCATCCGTAACCAAGAAATTTTGATTCCAACTCA
CTTCTTTATTGTGCTAACAAGCTGTAAAGATACATCTCAGACGCCTTTGCACTGTGAAAACCTAGACACCTTA
GCTTTCATTTTGCCTCACAGGACTGATAACAGCGAGAGCTGTGTGCATGGGAAGCATGACTCCTCATGGGTTG
AAGAATTGTTAATGTTACACAGAGCACGGATCACAGATGTTGAGCACATCACTGGACTCAGCTTCTATCAACA

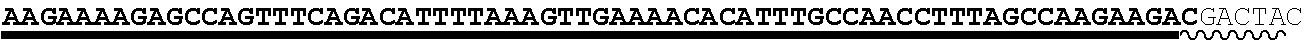

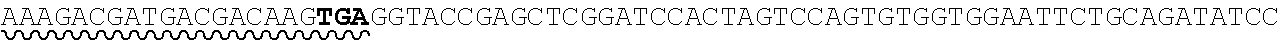

AGCACAGTGGCGGCCGCTCGAGTCTAGAGGGCCCGTTTAAACCCGCTGATCAGCCTCGACTGTGCCTTCTAGT
TGCCAGCCATCTGTTGTTTGCCCCTCCCCCGTGCCTTCCTTGACCCTGGAAGGTGCCACTCCCACTGTCCTTT
CCTAATAAAATGAGGAAATTGCATCGCATTGTCTGAGTAGGTGTCATTCTATTCTGGGGGGTGGGGTGGGGCA
GGACAGCAAGGGGGAGGATTGGGAAGACAATAGCAGGCATGCTGGGGATGCGGTGGGCTCTATGGCTTCTGAG
GCGGAAAGAACCAGCTGGGGCTCTAGGGGGTATCCCCACGCGCCCTGTAGCGGCGCATTAAGCGCGGCGGGTG
TGGTGGTTACGCGCAGCGTGACCGCTACACTTGCCAGCGCCCTAGCGCCCGCTCCTTTCGCTTTCTTCCCTTC
CTTTCTCGCCACGTTCGCCGGCTTTCCCCGTCAAGCTCTAAATCGGGGGCTCCCTTTAGGGTTCCGATTTAGT
GCTTTACGGCACCTCGACCCCAAAAAACTTGATTAGGGTGATGGTTCACGTAGTGGGCCATCGCCCTGATAGA
CGGTTTTTCGCCCTTTGACGTTGGAGTCCACGTTCTTTAATAGTGGACTCTTGTTCCAAACTGGAACAACACT
CAACCCTATCTCGGTCTATTCTTTTGATTTATAAGGGATTTTGCCGATTTCGGCCTATTGGTTAAAAAATGAG
CTGATTTAACAAAAATTTAACGCGAATTAATTCTGTGGAATGTGTGTCAGTTAGGGTGTGGAAAGTCCCCAGG
CTCCCCAGCAGGCAGAAGTATGCAAAGCATGCATCTCAATTAGTCAGCAACCAGGTGTGGAAAGTCCCCAGGC
TCCCCAGCAGGCAGAAGTATGCAAAGCATGCATCTCAATTAGTCAGCAACCATAGTCCCGCCCCTAACTCCGC
CCATCCCGCCCCTAACTCCGCCCAGTTCCGCCCATTCTCCGCCCCATGGCTGACTAATTTTTTTTATTTATGC
AGAGGCCGAGGCCGCCTCTGCCTCTGAGCTATTCCAGAAGTAGTGAGGAGGCTTTTTTGGAGGCCTAGGCTTT
TGCAAAAAGCTCCCGGGAGCTTGTATATCCATTTTCGGATCTGATCAAGAGACAGGATGAGGATCGTTTCGCA
TGATTGAACAAGATGGATTGCACGCAGGTTCTCCGGCCGCTTGGGTGGAGAGGCTATTCGGCTATGACTGGGC
ACAACAGACAATCGGCTGCTCTGATGCCGCCGTGTTCCGGCTGTCAGCGCAGGGGCGCCCGGTTCTTTTTGTC
AAGACCGACCTGTCCGGTGCCCTGAATGAACTGCAGGACGAGGCAGCGCGGCTATCGTGGCTGGCCACGACGG
GCGTTCCTTGCGCAGCTGTGCTCGACGTTGTCACTGAAGCGGGAAGGGACTGGCTGCTATTGGGCGAAGTGCC
GGGGCAGGATCTCCTGTCATCTCACCTTGCTCCTGCCGAGAAAGTATCCATCATGGCTGATGCAATGCGGCGG
CTGCATACGCTTGATCCGGCTACCTGCCCATTCGACCACCAAGCGAAACATCGCATCGAGCGAGCACGTACTC
GGATGGAAGCCGGTCTTGTCGATCAGGATGATCTGGACGAAGAGCATCAGGGGCTCGCGCCAGCCGAACTGTT
CGCCAGGCTCAAGGCGCGCATGCCCGACGGCGAGGATCTCGTCGTGACCCATGGCGATGCCTGCTTGCCGAAT
ATCATGGTGGAAAATGGCCGCTTTTCTGGATTCATCGACTGTGGCCGGCTGGGTGTGGCGGACCGCTATCAGG
ACATAGCGTTGGCTACCCGTGATATTGCTGAAGAGCTTGGCGGCGAATGGGCTGACCGCTTCCTCGTGCTTTA
CGGTATCGCCGCTCCCGATTCGCAGCGCATCGCCTTCTATCGCCTTCTTGACGAGTTCTTCTGAGCGGGACTC
TGGGGTTCGAAATGACCGACCAAGCGACGCCCAACCTGCCATCACGAGATTTCGATTCCACCGCCGCCTTCTA
TGAAAGGTTGGGCTTCGGAATCGTTTTCCGGGACGCCGGCTGGATGATCCTCCAGCGCGGGGATCTCATGCTG
GAGTTCTTCGCCCACCCCAACTTGTTTATTGCAGCTTATAATGGTTACAAATAAAGCAATAGCATCACAAATT
TCACAAATAAAGCATTTTTTTCACTGCATTCTAGTTGTGGTTTGTCCAAACTCATCAATGTATCTTATCATGT
CTGTATACCGTCGACCTCTAGCTAGAGCTTGGCGTAATCATGGTCATAGCTGTTTCCTGTGTGAAATTGTTAT
CCGCTCACAATTCCACACAACATACGAGCCGGAAGCATAAAGTGTAAAGCCTGGGGTGCCTAATGAGTGAGCT
AACTCACATTAATTGCGTTGCGCTCACTGCCCGCTTTCCAGTCGGGAAACCTGTCGTGCCAGCTGCATTAATG
AATCGGCCAACGCGCGGGGAGAGGCGGTTTGCGTATTGGGCGCTCTTCCGCTTCCTCGCTCACTGACTCGCTG
CGCTCGGTCGTTCGGCTGCGGCGAGCGGTATCAGCTCACTCAAAGGCGGTAATACGGTTATCCACAGAATCAG
GGGATAACGCAGGAAAGAACATGTGAGCAAAAGGCCAGCAAAAGGCCAGGAACCGTAAAAAGGCCGCGTTGCT
GGCGTTTTTCCATAGGCTCCGCCCCCCTGACGAGCATCACAAAAATCGACGCTCAAGTCAGAGGTGGCGAAAC
CCGACAGGACTATAAAGATACCAGGCGTTTCCCCCTGGAAGCTCCCTCGTGCGCTCTCCTGTTCCGACCCTGC
CGCTTACCGGATACCTGTCCGCCTTTCTCCCTTCGGGAAGCGTGGCGCTTTCTCATAGCTCACGCTGTAGGTA
TCTCAGTTCGGTGTAGGTCGTTCGCTCCAAGCTGGGCTGTGTGCACGAACCCCCCGTTCAGCCCGACCGCTGC
GCCTTATCCGGTAACTATCGTCTTGAGTCCAACCCGGTAAGACACGACTTATCGCCACTGGCAGCAGCCACTG
GTAACAGGATTAGCAGAGCGAGGTATGTAGGCGGTGCTACAGAGTTCTTGAAGTGGTGGCCTAACTACGGCTA
CACTAGAAGAACAGTATTTGGTATCTGCGCTCTGCTGAAGCCAGTTACCTTCGGAAAAAGAGTTGGTAGCTCT
TGATCCGGCAAACAAACCACCGCTGGTAGCGGTGGTTTTTTTGTTTGCAAGCAGCAGATTACGCGCAGAAAAA
AAGGATCTCAAGAAGATCCTTTGATCTTTTCTACGGGGTCTGACGCTCAGTGGAACGAAAACTCACGTTAAGG
GATTTTGGTCATGAGATTATCAAAAAGGATCTTCACCTAGATCCTTTTAAATTAAAAATGAAGTTTTAAATCA
ATCTAAAGTATATATGAGTAAACTTGGTCTGACAGTTACCAATGCTTAATCAGTGAGGCACCTATCTCAGCGA
TCTGTCTATTTCGTTCATCCATAGTTGCCTGACTCCCCGTCGTGTAGATAACTACGATACGGGAGGGCTTACC
ATCTGGCCCCAGTGCTGCAATGATACCGCGAGACCCACGCTCACCGGCTCCAGATTTATCAGCAATAAACCAG
CCAGCCGGAAGGGCCGAGCGCAGAAGTGGTCCTGCAACTTTATCCGCCTCCATCCAGTCTATTAATTGTTGCC
GGGAAGCTAGAGTAAGTAGTTCGCCAGTTAATAGTTTGCGCAACGTTGTTGCCATTGCTACAGGCATCGTGGT
GTCACGCTCGTCGTTTGGTATGGCTTCATTCAGCTCCGGTTCCCAACGATCAAGGCGAGTTACATGATCCCCC
ATGTTGTGCAAAAAAGCGGTTAGCTCCTTCGGTCCTCCGATCGTTGTCAGAAGTAAGTTGGCCGCAGTGTTAT
CACTCATGGTTATGGCAGCACTGCATAATTCTCTTACTGTCATGCCATCCGTAAGATGCTTTTCTGTGACTGG
TGAGTACTCAACCAAGTCATTCTGAGAATAGTGTATGCGGCGACCGAGTTGCTCTTGCCCGGCGTCAATACGG
GATAATACCGCGCCACATAGCAGAACTTTAAAAGTGCTCATCATTGGAAAACGTTCTTCGGGGCGAAAACTCT
CAAGGATCTTACCGCTGTTGAGATCCAGTTCGATGTAACCCACTCGTGCACCCAACTGATCTTCAGCATCTTT
TACTTTCACCAGCGTTTCTGGGTGAGCAAAAACAGGAAGGCAAAATGCCGCAAAAAAGGGAATAAGGGCGACA
CGGAAATGTTGAATACTCATACTCTTCCTTTTTCAATATTATTGAAGCATTTATCAGGGTTATTGTCTCATGA
GCGGATACATATTTGAATGTATTTAGAAAAATAAACAAATAGGGGTTCCGCGCACATTTCCCCGAAAAGTGCC
ACCTGACGTC
Soluble recombinant ENPP1 (srENPP1) constructs
(ENPP1opt is a codon optimized version of srENPP1)
CMV_kozak_hAlb sig_FLAG_ENPPlopt_halb_STOP_pcDNA3.1 (+)
(SEQ ID NO: 88)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTAGTTCCAGATGGTAAATATACACAAGGGATTTAGTCAAACAATTTTTTGGCAAGAATA
TTATGAATTTTGTAATCGGTTGGCAGCCAATGAAATACAAAGATGAGTCTAGTTAATAATCTACAATTATTGG
TTAAAGAAGTATATTAGTGCTAATTTCCCTCCGTTTGTCCTAGCTTTTCTCTTCTGTCAACCCCACACGCCTT
TGGCACCTCTGGCTAACTAGAGAACCCACTGCTTACTGGCTTATCGAAATTAATACGACTCACTATAGGGAGA
CCCAAGCTGGCTAGCGCCACCATGAAGTGGGTAACCTTTATTTCCCTTCTTTTTCTCTTTAGCTCGGCTTATT
CCAGGGGTGTGTTTCGTCGAGACTACAAAGACGATGACGACAAGAGCGCTGGCCTGAAGCCAAGCTGCGCCAA
GGAGGTGAAGAGCTGCAAGGGCAGATGTTTCGAACGGACCTTCGGCAACTGCAGATGCGACGCCGCCTGCGTG
GAACTGGGAAACTGTTGTCTGGACTACCAGGAGACATGTATCGAGCCTGAGCACATCTGGACATGCAACAAGT
TCAGATGTGGCGAGAAAAGACTGACTCGGAGTCTGTGCGCCTGTTCTGACGACTGCAAGGACAAGGGCGATTG
CTGCATCAACTACAGCAGCGTGTGCCAGGGCGAAAAGAGCTGGGTCGAGGAACCCTGCGAGAGCATCAACGAG
CCCCAGTGCCCTGCCGGATTTGAGACACCTCCTACCCTGCTCTTCTCCCTGGATGGCTTCAGAGCTGAGTACC
TGCATACATGGGGAGGACTGCTGCCTGTGATCAGCAAGCTGAAGAAGTGCGGCACCTACACAAAGAACATGCG
GCCTGTCTACCCTACCAAGACCTTCCCCAACCACTACAGCATCGTGACCGGCCTGTACCCCGAGAGCCACGGC
ATCATCGACAACAAGATGTACGACCCCAAAATGAACGCCAGCTTCAGCCTGAAATCTAAGGAAAAGTTCAACC
CTGAGTGGTATAAGGGCGAGCCAATCTGGGTGACAGCCAAGTACCAGGGCCTGAAGAGCGGCACCTTCTTCTG
GCCTGGCAGCGACGTGGAGATCAACGGAATCTTCCCTGATATCTACAAGATGTATAACGGCTCCGTGCCTTTC
GAGGAGCGGATCTTGGCTGTTCTTCAGTGGCTGCAGCTGCCCAAGGACGAGAGACCCCACTTCTACACCCTGT
ACCTGGAAGAACCTGACAGCAGCGGCCACAGCTACGGCCCTGTGTCTAGCGAGGTGATCAAGGCCCTGCAAAG
AGTGGATGGCATGGTGGGCATGCTGATGGATGGACTGAAGGAACTGAACCTCCATCGTTGCCTGAACCTGATC
CTGATCTCCGACCACGGCATGGAACAGGGCAGCTGTAAAAAGTACATCTACCTGAACAAGTACCTGGGCGACG
TGAAGAATATCAAGGTGATCTACGGGCCGGCCGCCAGGCTGCGGCCTAGCGACGTGCCCGACAAGTATTACAG
CTTCAACTACGAGGGCATCGCCAGAAACCTGTCTTGCAGAGAACCCAATCAACACTTCAAGCCTTACCTGAAG
CACTTTCTGCCTAAGAGACTGCACTTCGCCAAAAGCGACAGAATTGAACCCCTGACATTCTATCTGGACCCTC
AGTGGCAGCTGGCCCTCAATCCTTCTGAACGCAAATACTGCGGTAGCGGATTTCACGGCTCTGATAACGTGTT
TTCTAACATGCAGGCCCTGTTTGTGGGCTACGGCCCTGGCTTCAAGCACGGAATCGAGGCTGACACCTTCGAG
AACATCGAAGTTTACAATCTGATGTGCGACCTGCTGAATCTGACCCCAGCCCCTAACAACGGCACACACGGCT
CTCTGAATCACCTGCTCAAGAACCCCGTGTATACCCCCAAACACCCTAAAGAGGTCCATCCACTGGTGCAGTG
CCCATTCACCAGAAACCCTCGGGACAACCTTGGCTGCAGCTGCAACCCTAGCATCCTCCCTATCGAAGATTTC
CAGACCCAGTTTAACCTGACTGTCGCCGAAGAGAAGATCATCAAGCACGAAACCCTGCCCTACGGGAGGCCCA
GAGTGCTGCAGAAAGAAAACACAATCTGTCTGCTGAGCCAGCACCAGTTTATGAGCGGCTACAGTCAGGACAT
CCTGATGCCTCTGTGGACCAGCTACACCGTGGACCGGAATGACAGCTTCAGCACCGAGGACTTCAGCAACTGC
CTGTATCAGGACTTCCGCATCCCTCTGTCCCCTGTGCATAAGTGCAGCTTTTACAAGAACAACACCAAGGTGA
GCTACGGGTTTCTGAGCCCACCACAACTGAACAAAAATAGCTCTGGCATCTACTCTGAGGCGCTGCTGACCAC
CAACATCGTGCCAATGTACCAGAGCTTCCAGGTGATCTGGCGGTACTTCCATGATACCCTGCTTCGGAAGTAC
GCCGAAGAACGGAACGGCGTGAACGTGGTGTCCGGCCCAGTGTTCGACTTCGACTACGACGGCAGATGTGATT
CCCTTGAGAATCTGAGACAGAAGCGGCGGGTTATCAGAAACCAGGAGATCCTGATTCCTACCCACTTCTTCAT
CGTGCTGACATCTTGCAAGGATACCAGCCAAACACCTCTGCACTGCGAGAACCTGGACACCCTGGCCTTCATC
CTGCCTCACCGGACCGATAATAGCGAATCCTGTGTGCACGGCAAACACGACTCTAGCTGGGTGGAAGAGCTGC
TGATGCTGCACAGAGCCCGGATCACAGATGTGGAACACATCACCGGCCTGTCCTTCTACCAACAGAGAAAGGA
ACCCGTGTCTGATATCCTGAAGCTGAAAACCCACCTGCCTACCTTCAGCCAGGAGGACCTGATCGTTAACGAT
GCACACAAGAGTGAGGTTGCTCATCGGTTTAAAGATTTGGGAGAAGAAAATTTCAAAGCCTTGGTGTTGATTG
CCTTTGCTCAGTATCTTCAGCAGTGTCCATTTGAAGATCATGTAAAATTAGTGAATGAAGTAACTGAATTTGC
AAAAACATGTGTTGCTGATGAGTCAGCTGAAAATTGTGACAAATCACTTCATACCCTTTTTGGAGACAAATTA
TGCACAGTTGCAACTCTTCGTGAAACCTATGGTGAAATGGCTGACTGCTGTGCAAAACAAGAACCTGAGAGAA
ATGAATGCTTCTTGCAACACAAAGATGACAACCCAAACCTCCCCCGATTGGTGAGACCAGAGGTTGATGTGAT
GTGCACTGCTTTTCATGACAATGAAGAGACATTTTTGAAAAAATACTTATATGAAATTGCCAGAAGACATCCT
TACTTTTATGCCCCGGAACTCCTTTTCTTTGCTAAAAGGTATAAAGCTGCTTTTACAGAATGTTGCCAAGCTG
CTGATAAAGCTGCCTGCCTGTTGCCAAAGCTCGATGAACTTCGGGATGAAGGGAAGGCTTCGTCTGCCAAACA
GAGACTCAAGTGTGCCAGTCTCCAAAAATTTGGAGAAAGAGCTTTCAAAGCATGGGCAGTAGCTCGCCTGAGC
CAGAGATTTCCCAAAGCTGAGTTTGCAGAAGTTTCCAAGTTAGTGACAGATCTTACCAAAGTCCACACGGAAT
GCTGCCATGGAGATCTGCTTGAATGTGCTGATGACAGGGCGGACCTTGCCAAGTATATCTGTGAAAATCAAGA
TTCGATCTCCAGTAAACTGAAGGAATGCTGTGAAAAACCTCTGTTGGAAAAATCCCACTGCATTGCCGAAGTG
GAAAATGATGAGATGCCTGCTGACTTGCCTTCATTAGCTGCTGATTTTGTTGAAAGTAAGGATGTTTGCAAAA
ACTATGCTGAGGCAAAGGATGTCTTCCTGGGCATGTTTTTGTATGAATATGCAAGAAGGCATCCTGATTACTC
TGTCGTGCTGCTGCTGAGACTTGCCAAGACATATGAAACCACACTCGAGAAGTGCTGTGCCGCTGCAGATCCT
CATGAATGCTATGCCAAAGTGTTCGATGAATTTAAACCTCTTGTGGAAGAGCCTCAGAATTTAATCAAACAAA
ATTGTGAGCTTTTTGAGCAGCTTGGAGAGTACAAATTCCAGAATGCGCTATTAGTTCGTTACACCAAGAAAGT
ACCCCAAGTGTCAACTCCAACTCTTGTAGAGGTCTCAAGAAACCTAGGAAAAGTGGGCAGCAAATGTTGTAAA
CATCCTGAAGCAAAAAGAATGCCCTGTGCAGAAGACTATCTATCCGTGGTCCTGAACCAGTTATGTGTGTTGC
ATGAGAAAACGCCAGTAAGTGACAGAGTCACCAAATGCTGCACAGAATCCTTGGTGAACAGGCGACCATGCTT
TTCAGCTCTGGAAGTCGATGAAACATACGTTCCCAAAGAGTTTAATGCTGAAACATTCACCTTCCATGCAGAT
ATATGCACACTTTCTGAGAAGGAGAGACAAATCAAGAAACAAACTGCACTTGTTGAGCTCGTGAAACACAAGC
CCAAGGCAACAAAAGAGCAACTGAAAGCTGTTATGGATGATTTCGCAGCTTTTGTAGAGAAGTGCTGCAAGGC
TGACGATAAGGAGACCTGCTTTGCCGAGGAGGGTAAAAAACTTGTTGCTGCAAGTCAAGCTGCCTTAGGCTTA
TGAGGTACCGAGCTCGGATCCACTAGTCCAGTGTGGTGGAATTCTGCAGATATCCAGCACAGTGGCGGCCGCT
CGAGTCTAGAGGGCCCGTTTAAACCCGCTGATCAGCCTCGACTGTGCCTTCTAGTTGCCAGCCATCTGTTGTT
TGCCCCTCCCCCGTGCCTTCCTTGACCCTGGAAGGTGCCACTCCCACTGTCCTTTCCTAATAAAATGAGGAAA
TTGCATCGCATTGTCTGAGTAGGTGTCATTCTATTCTGGGGGGTGGGGTGGGGCAGGACAGCAAGGGGGAGGA
TTGGGAAGACAATAGCAGGCATGCTGGGGATGCGGTGGGCTCTATGGCTTCTGAGGCGGAAAGAACCAGCTGG
GGCTCTAGGGGGTATCCCCACGCGCCCTGTAGCGGCGCATTAAGCGCGGCGGGTGTGGTGGTTACGCGCAGCG
TGACCGCTACACTTGCCAGCGCCCTAGCGCCCGCTCCTTTCGCTTTCTTCCCTTCCTTTCTCGCCACGTTCGC
CGGCTTTCCCCGTCAAGCTCTAAATCGGGGGCTCCCTTTAGGGTTCCGATTTAGTGCTTTACGGCACCTCGAC
CCCAAAAAACTTGATTAGGGTGATGGTTCACGTAGTGGGCCATCGCCCTGATAGACGGTTTTTCGCCCTTTGA
CGTTGGAGTCCACGTTCTTTAATAGTGGACTCTTGTTCCAAACTGGAACAACACTCAACCCTATCTCGGTCTA
TTCTTTTGATTTATAAGGGATTTTGCCGATTTCGGCCTATTGGTTAAAAAATGAGCTGATTTAACAAAAATTT
AACGCGAATTAATTCTGTGGAATGTGTGTCAGTTAGGGTGTGGAAAGTCCCCAGGCTCCCCAGCAGGCAGAAG
TATGCAAAGCATGCATCTCAATTAGTCAGCAACCAGGTGTGGAAAGTCCCCAGGCTCCCCAGCAGGCAGAAGT
ATGCAAAGCATGCATCTCAATTAGTCAGCAACCATAGTCCCGCCCCTAACTCCGCCCATCCCGCCCCTAACTC
CGCCCAGTTCCGCCCATTCTCCGCCCCATGGCTGACTAATTTTTTTTATTTATGCAGAGGCCGAGGCCGCCTC
TGCCTCTGAGCTATTCCAGAAGTAGTGAGGAGGCTTTTTTGGAGGCCTAGGCTTTTGCAAAAAGCTCCCGGGA
GCTTGTATATCCATTTTCGGATCTGATCAAGAGACAGGATGAGGATCGTTTCGCATGATTGAACAAGATGGAT
TGCACGCAGGTTCTCCGGCCGCTTGGGTGGAGAGGCTATTCGGCTATGACTGGGCACAACAGACAATCGGCTG
CTCTGATGCCGCCGTGTTCCGGCTGTCAGCGCAGGGGCGCCCGGTTCTTTTTGTCAAGACCGACCTGTCCGGT
GCCCTGAATGAACTGCAGGACGAGGCAGCGCGGCTATCGTGGCTGGCCACGACGGGCGTTCCTTGCGCAGCTG
TGCTCGACGTTGTCACTGAAGCGGGAAGGGACTGGCTGCTATTGGGCGAAGTGCCGGGGCAGGATCTCCTGTC
ATCTCACCTTGCTCCTGCCGAGAAAGTATCCATCATGGCTGATGCAATGCGGCGGCTGCATACGCTTGATCCG
GCTACCTGCCCATTCGACCACCAAGCGAAACATCGCATCGAGCGAGCACGTACTCGGATGGAAGCCGGTCTTG
TCGATCAGGATGATCTGGACGAAGAGCATCAGGGGCTCGCGCCAGCCGAACTGTTCGCCAGGCTCAAGGCGCG
CATGCCCGACGGCGAGGATCTCGTCGTGACCCATGGCGATGCCTGCTTGCCGAATATCATGGTGGAAAATGGC
CGCTTTTCTGGATTCATCGACTGTGGCCGGCTGGGTGTGGCGGACCGCTATCAGGACATAGCGTTGGCTACCC
GTGATATTGCTGAAGAGCTTGGCGGCGAATGGGCTGACCGCTTCCTCGTGCTTTACGGTATCGCCGCTCCCGA
TTCGCAGCGCATCGCCTTCTATCGCCTTCTTGACGAGTTCTTCTGAGCGGGACTCTGGGGTTCGAAATGACCG
ACCAAGCGACGCCCAACCTGCCATCACGAGATTTCGATTCCACCGCCGCCTTCTATGAAAGGTTGGGCTTCGG
AATCGTTTTCCGGGACGCCGGCTGGATGATCCTCCAGCGCGGGGATCTCATGCTGGAGTTCTTCGCCCACCCC
AACTTGTTTATTGCAGCTTATAATGGTTACAAATAAAGCAATAGCATCACAAATTTCACAAATAAAGCATTTT
TTTCACTGCATTCTAGTTGTGGTTTGTCCAAACTCATCAATGTATCTTATCATGTCTGTATACCGTCGACCTC
TAGCTAGAGCTTGGCGTAATCATGGTCATAGCTGTTTCCTGTGTGAAATTGTTATCCGCTCACAATTCCACAC
AACATACGAGCCGGAAGCATAAAGTGTAAAGCCTGGGGTGCCTAATGAGTGAGCTAACTCACATTAATTGCGT
TGCGCTCACTGCCCGCTTTCCAGTCGGGAAACCTGTCGTGCCAGCTGCATTAATGAATCGGCCAACGCGCGGG
GAGAGGCGGTTTGCGTATTGGGCGCTCTTCCGCTTCCTCGCTCACTGACTCGCTGCGCTCGGTCGTTCGGCTG
CGGCGAGCGGTATCAGCTCACTCAAAGGCGGTAATACGGTTATCCACAGAATCAGGGGATAACGCAGGAAAGA
ACATGTGAGCAAAAGGCCAGCAAAAGGCCAGGAACCGTAAAAAGGCCGCGTTGCTGGCGTTTTTCCATAGGCT
CCGCCCCCCTGACGAGCATCACAAAAATCGACGCTCAAGTCAGAGGTGGCGAAACCCGACAGGACTATAAAGA
TACCAGGCGTTTCCCCCTGGAAGCTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGTAGGTATCTCAGTTCGGTGTAGGT
CGTTCGCTCCAAGCTGGGCTGTGTGCACGAACCCCCCGTTCAGCCCGACCGCTGCGCCTTATCCGGTAACTAT
CGTCTTGAGTCCAACCCGGTAAGACACGACTTATCGCCACTGGCAGCAGCCACTGGTAACAGGATTAGCAGAG
CGAGGTATGTAGGCGGTGCTACAGAGTTCTTGAAGTGGTGGCCTAACTACGGCTACACTAGAAGAACAGTATT
TGGTATCTGCGCTCTGCTGAAGCCAGTTACCTTCGGAAAAAGAGTTGGTAGCTCTTGATCCGGCAAACAAACC
ACCGCTGGTAGCGGTGGTTTTTTTGTTTGCAAGCAGCAGATTACGCGCAGAAAAAAAGGATCTCAAGAAGATC
CTTTGATCTTTTCTACGGGGTCTGACGCTCAGTGGAACGAAAACTCACGTTAAGGGATTTTGGTCATGAGATT
ATCAAAAAGGATCTTCACCTAGATCCTTTTAAATTAAAAATGAAGTTTTAAATCAATCTAAAGTATATATGAG
TAAACTTGGTCTGACAGTTACCAATGCTTAATCAGTGAGGCACCTATCTCAGCGATCTGTCTATTTCGTTCAT
CCATAGTTGCCTGACTCCCCGTCGTGTAGATAACTACGATACGGGAGGGCTTACCATCTGGCCCCAGTGCTGC
AATGATACCGCGAGACCCACGCTCACCGGCTCCAGATTTATCAGCAATAAACCAGCCAGCCGGAAGGGCCGAG
CGCAGAAGTGGTCCTGCAACTTTATCCGCCTCCATCCAGTCTATTAATTGTTGCCGGGAAGCTAGAGTAAGTA
GTTCGCCAGTTAATAGTTTGCGCAACGTTGTTGCCATTGCTACAGGCATCGTGGTGTCACGCTCGTCGTTTGG
TATGGCTTCATTCAGCTCCGGTTCCCAACGATCAAGGCGAGTTACATGATCCCCCATGTTGTGCAAAAAAGCG
GTTAGCTCCTTCGGTCCTCCGATCGTTGTCAGAAGTAAGTTGGCCGCAGTGTTATCACTCATGGTTATGGCAG
CACTGCATAATTCTCTTACTGTCATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAATACGGGATAATACCGCGCCACAT
AGCAGAACTTTAAAAGTGCTCATCATTGGAAAACGTTCTTCGGGGCGAAAACTCTCAAGGATCTTACCGCTGT
TGAGATCCAGTTCGATGTAACCCACTCGTGCACCCAACTGATCTTCAGCATCTTTTACTTTCACCAGCGTTTC
TGGGTGAGCAAAAACAGGAAGGCAAAATGCCGCAAAAAAGGGAATAAGGGCGACACGGAAATGTTGAATACTC
ATACTCTTCCTTTTTCAATATTATTGAAGCATTTATCAGGGTTATTGTCTCATGAGCGGATACATATTTGAAT
GTATTTAGAAAAATAAACAAATAGGGGTTCCGCGCACATTTCCCCGAAAAGTGCCACCTGACGTC
CMV_kozak_hAlb sig_FLAG_ENPPlopt_halb_STOP_MIR155_pcDNA3.1 (+)
(SEQ ID NO: 89)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTGACATTGATTATTGACTAGTTATTAATAGTAATCAATTACGGGGTCATTAGTTCATAG
CCCATATATGGAGTTCCGCGTTACATAACTTACGGTAAATGGCCCGCCTGGCTGACCGCCCAACGACCCCCGC
CCATTGACGTCAATAATGACGTATGTTCCCATAGTAACGCCAATAGGGACTTTCCATTGACGTCAATGGGTGG
AGTATTTACGGTAAACTGCCCACTTGGCAGTACATCAAGTGTATCATATGCCAAGTACGCCCCCTATTGACGT
CAATGACGGTAAATGGCCCGCCTGGCATTATGCCCAGTACATGACCTTATGGGACTTTCCTACTTGGCAGTAC
ATCTACGTATTAGTCATCGCTATTACCATGGTGATGCGGTTTTGGCAGTACATCAATGGGCGTGGATAGCGGT
TTGACTCACGGGGATTTCCAAGTCTCCACCCCATTGACGTCAATGGGAGTTTGTTTTGGCACCAAAATCAACG
GGACTTTCCAAAATGTCGTAACAACTCCGCCCCATTGACGCAAATGGGCGGTAGGCGTGTACGGTGGGAGGTC
TATATAAGCAGAGCTCTCTGGCTAACTAGAGAACCCACTGCTTACTGGCTTATCGAAATTAATACGACTCACT
ATAGGGAGACCCAAGCTGGCTAGCGCCACCATGAAGTGGGTAACCTTTATTTCCCTTCTTTTTCTCTTTAGCT
CGGCTTATTCCAGGGGTGTGTTTCGTCGAGACTACAAAGACGATGACGACAAGAGCGCTGGCCTGAAGCCAAG
CTGCGCCAAGGAGGTGAAGAGCTGCAAGGGCAGATGTTTCGAACGGACCTTCGGCAACTGCAGATGCGACGCC
GCCTGCGTGGAACTGGGAAACTGTTGTCTGGACTACCAGGAGACATGTATCGAGCCTGAGCACATCTGGACAT
GCAACAAGTTCAGATGTGGCGAGAAAAGACTGACTCGGAGTCTGTGCGCCTGTTCTGACGACTGCAAGGACAA
GGGCGATTGCTGCATCAACTACAGCAGCGTGTGCCAGGGCGAAAAGAGCTGGGTCGAGGAACCCTGCGAGAGC
ATCAACGAGCCCCAGTGCCCTGCCGGATTTGAGACACCTCCTACCCTGCTCTTCTCCCTGGATGGCTTCAGAG
CTGAGTACCTGCATACATGGGGAGGACTGCTGCCTGTGATCAGCAAGCTGAAGAAGTGCGGCACCTACACAAA
GAACATGCGGCCTGTCTACCCTACCAAGACCTTCCCCAACCACTACAGCATCGTGACCGGCCTGTACCCCGAG
AGCCACGGCATCATCGACAACAAGATGTACGACCCCAAAATGAACGCCAGCTTCAGCCTGAAATCTAAGGAAA
AGTTCAACCCTGAGTGGTATAAGGGCGAGCCAATCTGGGTGACAGCCAAGTACCAGGGCCTGAAGAGCGGCAC
CTTCTTCTGGCCTGGCAGCGACGTGGAGATCAACGGAATCTTCCCTGATATCTACAAGATGTATAACGGCTCC
GTGCCTTTCGAGGAGCGGATCTTGGCTGTTCTTCAGTGGCTGCAGCTGCCCAAGGACGAGAGACCCCACTTCT
ACACCCTGTACCTGGAAGAACCTGACAGCAGCGGCCACAGCTACGGCCCTGTGTCTAGCGAGGTGATCAAGGC
CCTGCAAAGAGTGGATGGCATGGTGGGCATGCTGATGGATGGACTGAAGGAACTGAACCTCCATCGTTGCCTG
AACCTGATCCTGATCTCCGACCACGGCATGGAACAGGGCAGCTGTAAAAAGTACATCTACCTGAACAAGTACC
TGGGCGACGTGAAGAATATCAAGGTGATCTACGGGCCGGCCGCCAGGCTGCGGCCTAGCGACGTGCCCGACAA
GTATTACAGCTTCAACTACGAGGGCATCGCCAGAAACCTGTCTTGCAGAGAACCCAATCAACACTTCAAGCCT
TACCTGAAGCACTTTCTGCCTAAGAGACTGCACTTCGCCAAAAGCGACAGAATTGAACCCCTGACATTCTATC
TGGACCCTCAGTGGCAGCTGGCCCTCAATCCTTCTGAACGCAAATACTGCGGTAGCGGATTTCACGGCTCTGA
TAACGTGTTTTCTAACATGCAGGCCCTGTTTGTGGGCTACGGCCCTGGCTTCAAGCACGGAATCGAGGCTGAC
ACCTTCGAGAACATCGAAGTTTACAATCTGATGTGCGACCTGCTGAATCTGACCCCAGCCCCTAACAACGGCA
CACACGGCTCTCTGAATCACCTGCTCAAGAACCCCGTGTATACCCCCAAACACCCTAAAGAGGTCCATCCACT
GGTGCAGTGCCCATTCACCAGAAACCCTCGGGACAACCTTGGCTGCAGCTGCAACCCTAGCATCCTCCCTATC
GAAGATTTCCAGACCCAGTTTAACCTGACTGTCGCCGAAGAGAAGATCATCAAGCACGAAACCCTGCCCTACG
GGAGGCCCAGAGTGCTGCAGAAAGAAAACACAATCTGTCTGCTGAGCCAGCACCAGTTTATGAGCGGCTACAG
TCAGGACATCCTGATGCCTCTGTGGACCAGCTACACCGTGGACCGGAATGACAGCTTCAGCACCGAGGACTTC
AGCAACTGCCTGTATCAGGACTTCCGCATCCCTCTGTCCCCTGTGCATAAGTGCAGCTTTTACAAGAACAACA
CCAAGGTGAGCTACGGGTTTCTGAGCCCACCACAACTGAACAAAAATAGCTCTGGCATCTACTCTGAGGCGCT
GCTGACCACCAACATCGTGCCAATGTACCAGAGCTTCCAGGTGATCTGGCGGTACTTCCATGATACCCTGCTT
CGGAAGTACGCCGAAGAACGGAACGGCGTGAACGTGGTGTCCGGCCCAGTGTTCGACTTCGACTACGACGGCA
GATGTGATTCCCTTGAGAATCTGAGACAGAAGCGGCGGGTTATCAGAAACCAGGAGATCCTGATTCCTACCCA
CTTCTTCATCGTGCTGACATCTTGCAAGGATACCAGCCAAACACCTCTGCACTGCGAGAACCTGGACACCCTG
GCCTTCATCCTGCCTCACCGGACCGATAATAGCGAATCCTGTGTGCACGGCAAACACGACTCTAGCTGGGTGG
AAGAGCTGCTGATGCTGCACAGAGCCCGGATCACAGATGTGGAACACATCACCGGCCTGTCCTTCTACCAACA
GAGAAAGGAACCCGTGTCTGATATCCTGAAGCTGAAAACCCACCTGCCTACCTTCAGCCAGGAGGACCTGATC
GTTAACGATGCACACAAGAGTGAGGTTGCTCATCGGTTTAAAGATTTGGGAGAAGAAAATTTCAAAGCCTTGG
TGTTGATTGCCTTTGCTCAGTATCTTCAGCAGTGTCCATTTGAAGATCATGTAAAATTAGTGAATGAAGTAAC
TGAATTTGCAAAAACATGTGTTGCTGATGAGTCAGCTGAAAATTGTGACAAATCACTTCATACCCTTTTTGGA
GACAAATTATGCACAGTTGCAACTCTTCGTGAAACCTATGGTGAAATGGCTGACTGCTGTGCAAAACAAGAAC
CTGAGAGAAATGAATGCTTCTTGCAACACAAAGATGACAACCCAAACCTCCCCCGATTGGTGAGACCAGAGGT
TGATGTGATGTGCACTGCTTTTCATGACAATGAAGAGACATTTTTGAAAAAATACTTATATGAAATTGCCAGA
AGACATCCTTACTTTTATGCCCCGGAACTCCTTTTCTTTGCTAAAAGGTATAAAGCTGCTTTTACAGAATGTT
GCCAAGCTGCTGATAAAGCTGCCTGCCTGTTGCCAAAGCTCGATGAACTTCGGGATGAAGGGAAGGCTTCGTC
TGCCAAACAGAGACTCAAGTGTGCCAGTCTCCAAAAATTTGGAGAAAGAGCTTTCAAAGCATGGGCAGTAGCT
CGCCTGAGCCAGAGATTTCCCAAAGCTGAGTTTGCAGAAGTTTCCAAGTTAGTGACAGATCTTACCAAAGTCC
ACACGGAATGCTGCCATGGAGATCTGCTTGAATGTGCTGATGACAGGGCGGACCTTGCCAAGTATATCTGTGA
AAATCAAGATTCGATCTCCAGTAAACTGAAGGAATGCTGTGAAAAACCTCTGTTGGAAAAATCCCACTGCATT
GCCGAAGTGGAAAATGATGAGATGCCTGCTGACTTGCCTTCATTAGCTGCTGATTTTGTTGAAAGTAAGGATG
TTTGCAAAAACTATGCTGAGGCAAAGGATGTCTTCCTGGGCATGTTTTTGTATGAATATGCAAGAAGGCATCC
TGATTACTCTGTCGTGCTGCTGCTGAGACTTGCCAAGACATATGAAACCACACTCGAGAAGTGCTGTGCCGCT
GCAGATCCTCATGAATGCTATGCCAAAGTGTTCGATGAATTTAAACCTCTTGTGGAAGAGCCTCAGAATTTAA
TCAAACAAAATTGTGAGCTTTTTGAGCAGCTTGGAGAGTACAAATTCCAGAATGCGCTATTAGTTCGTTACAC
CAAGAAAGTACCCCAAGTGTCAACTCCAACTCTTGTAGAGGTCTCAAGAAACCTAGGAAAAGTGGGCAGCAAA
TGTTGTAAACATCCTGAAGCAAAAAGAATGCCCTGTGCAGAAGACTATCTATCCGTGGTCCTGAACCAGTTAT
GTGTGTTGCATGAGAAAACGCCAGTAAGTGACAGAGTCACCAAATGCTGCACAGAATCCTTGGTGAACAGGCG
ACCATGCTTTTCAGCTCTGGAAGTCGATGAAACATACGTTCCCAAAGAGTTTAATGCTGAAACATTCACCTTC
CATGCAGATATATGCACACTTTCTGAGAAGGAGAGACAAATCAAGAAACAAACTGCACTTGTTGAGCTCGTGA
AACACAAGCCCAAGGCAACAAAAGAGCAACTGAAAGCTGTTATGGATGATTTCGCAGCTTTTGTAGAGAAGTG
CTGCAAGGCTGACGATAAGGAGACCTGCTTTGCCGAGGAGGGTAAAAAACTTGTTGCTGCAAGTCAAGCTGCC
TTAGGCTTATGACAATTACGATTAGCACTATCCAATTACGATTAGCACTATCCAATTACGATTAGCACTATCG
GTACCGAGCTCGGATCCACTAGTCCAGTGTGGTGGAATTCTGCAGATATCCAGCACAGTGGCGGCCGCTCGAG
TCTAGAGGGCCCGTTTAAACCCGCTGATCAGCCTCGACTGTGCCTTCTAGTTGCCAGCCATCTGTTGTTTGCC
CCTCCCCCGTGCCTTCCTTGACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAGGACAGCAAGGGGGAGGATTGG
GAAGACAATAGCAGGCATGCTGGGGATGCGGTGGGCTCTATGGCTTCTGAGGCGGAAAGAACCAGCTGGGGCT
CTAGGGGGTATCCCCACGCGCCCTGTAGCGGCGCATTAAGCGCGGCGGGTGTGGTGGTTACGCGCAGCGTGAC
CGCTACACTTGCCAGCGCCCTAGCGCCCGCTCCTTTCGCTTTCTTCCCTTCCTTTCTCGCCACGTTCGCCGGC
TTTCCCCGTCAAGCTCTAAATCGGGGGCTCCCTTTAGGGTTCCGATTTAGTGCTTTACGGCACCTCGACCCCA
AAAAACTTGATTAGGGTGATGGTTCACGTAGTGGGCCATCGCCCTGATAGACGGTTTTTCGCCCTTTGACGTT
GGAGTCCACGTTCTTTAATAGTGGACTCTTGTTCCAAACTGGAACAACACTCAACCCTATCTCGGTCTATTCT
TTTGATTTATAAGGGATTTTGCCGATTTCGGCCTATTGGTTAAAAAATGAGCTGATTTAACAAAAATTTAACG
CGAATTAATTCTGTGGAATGTGTGTCAGTTAGGGTGTGGAAAGTCCCCAGGCTCCCCAGCAGGCAGAAGTATG
CAAAGCATGCATCTCAATTAGTCAGCAACCAGGTGTGGAAAGTCCCCAGGCTCCCCAGCAGGCAGAAGTATGC
AAAGCATGCATCTCAATTAGTCAGCAACCATAGTCCCGCCCCTAACTCCGCCCATCCCGCCCCTAACTCCGCC
CAGTTCCGCCCATTCTCCGCCCCATGGCTGACTAATTTTTTTTATTTATGCAGAGGCCGAGGCCGCCTCTGCC
TCTGAGCTATTCCAGAAGTAGTGAGGAGGCTTTTTTGGAGGCCTAGGCTTTTGCAAAAAGCTCCCGGGAGCTT
GTATATCCATTTTCGGATCTGATCAAGAGACAGGATGAGGATCGTTTCGCATGATTGAACAAGATGGATTGCA
CGCAGGTTCTCCGGCCGCTTGGGTGGAGAGGCTATTCGGCTATGACTGGGCACAACAGACAATCGGCTGCTCT
GATGCCGCCGTGTTCCGGCTGTCAGCGCAGGGGCGCCCGGTTCTTTTTGTCAAGACCGACCTGTCCGGTGCCC
TGAATGAACTGCAGGACGAGGCAGCGCGGCTATCGTGGCTGGCCACGACGGGCGTTCCTTGCGCAGCTGTGCT
CGACGTTGTCACTGAAGCGGGAAGGGACTGGCTGCTATTGGGCGAAGTGCCGGGGCAGGATCTCCTGTCATCT
CACCTTGCTCCTGCCGAGAAAGTATCCATCATGGCTGATGCAATGCGGCGGCTGCATACGCTTGATCCGGCTA
CCTGCCCATTCGACCACCAAGCGAAACATCGCATCGAGCGAGCACGTACTCGGATGGAAGCCGGTCTTGTCGA
TCAGGATGATCTGGACGAAGAGCATCAGGGGCTCGCGCCAGCCGAACTGTTCGCCAGGCTCAAGGCGCGCATG
CCCGACGGCGAGGATCTCGTCGTGACCCATGGCGATGCCTGCTTGCCGAATATCATGGTGGAAAATGGCCGCT
TTTCTGGATTCATCGACTGTGGCCGGCTGGGTGTGGCGGACCGCTATCAGGACATAGCGTTGGCTACCCGTGA
TATTGCTGAAGAGCTTGGCGGCGAATGGGCTGACCGCTTCCTCGTGCTTTACGGTATCGCCGCTCCCGATTCG
CAGCGCATCGCCTTCTATCGCCTTCTTGACGAGTTCTTCTGAGCGGGACTCTGGGGTTCGAAATGACCGACCA
AGCGACGCCCAACCTGCCATCACGAGATTTCGATTCCACCGCCGCCTTCTATGAAAGGTTGGGCTTCGGAATC
GTTTTCCGGGACGCCGGCTGGATGATCCTCCAGCGCGGGGATCTCATGCTGGAGTTCTTCGCCCACCCCAACT
TGTTTATTGCAGCTTATAATGGTTACAAATAAAGCAATAGCATCACAAATTTCACAAATAAAGCATTTTTTTC
ACTGCATTCTAGTTGTGGTTTGTCCAAACTCATCAATGTATCTTATCATGTCTGTATACCGTCGACCTCTAGC
TAGAGCTTGGCGTAATCATGGTCATAGCTGTTTCCTGTGTGAAATTGTTATCCGCTCACAATTCCACACAACA
TACGAGCCGGAAGCATAAAGTGTAAAGCCTGGGGTGCCTAATGAGTGAGCTAACTCACATTAATTGCGTTGCG
CTCACTGCCCGCTTTCCAGTCGGGAAACCTGTCGTGCCAGCTGCATTAATGAATCGGCCAACGCGCGGGGAGA
GGCGGTTTGCGTATTGGGCGCTCTTCCGCTTCCTCGCTCACTGACTCGCTGCGCTCGGTCGTTCGGCTGCGGC
GAGCGGTATCAGCTCACTCAAAGGCGGTAATACGGTTATCCACAGAATCAGGGGATAACGCAGGAAAGAACAT
GTGAGCAAAAGGCCAGCAAAAGGCCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAACCCGACAGGACTATAAAGATACC
AGGCGTTTCCCCCTGGAAGCTCCCTCGTGCGCTCTCCTGTTCCGACCCTGCCGCTTACCGGATACCTGTCCGC
CTTTCTCCCTTCGGGAAGCGTGGCGCTTTCTCATAGCTCACGCTGTAGGTATCTCAGTTCGGTGTAGGTCGTT
CGCTCCAAGCTGGGCTGTGTGCACGAACCCCCCGTTCAGCCCGACCGCTGCGCCTTATCCGGTAACTATCGTC
TTGAGTCCAACCCGGTAAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAGAAGAACAGTATTTGGT
ATCTGCGCTCTGCTGAAGCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGATCTCAAGAAGATCCTTT
GATCTTTTCTACGGGGTCTGACGCTCAGTGGAACGAAAACTCACGTTAAGGGATTTTGGTCATGAGATTATCA
AAAAGGATCTTCACCTAGATCCTTTTAAATTAAAAATGAAGTTTTAAATCAATCTAAAGTATATATGAGTAAA
CTTGGTCTGACAGTTACCAATGCTTAATCAGTGAGGCACCTATCTCAGCGATCTGTCTATTTCGTTCATCCAT
AGTTGCCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGAGCGCA
GAAGTGGTCCTGCAACTTTATCCGCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGGTGTCACGCTCGTCGTTTGGTATG
GCTTCATTCAGCTCCGGTTCCCAACGATCAAGGCGAGTTACATGATCCCCCATGTTGTGCAAAAAAGCGGTTA
GCTCCTTCGGTCCTCCGATCGTTGTCAGAAGTAAGTTGGCCGCAGTGTTATCACTCATGGTTATGGCAGCACT
GCATAATTCTCTTACTGTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAATACCGCGCCACATAGCA
GAACTTTAAAAGTGCTCATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACACGGAAATGTTGAATACTCATAC
TCTTCCTTTTTCAATATTATTGAAGCATTTATCAGGGTTATTGTCTCATGAGCGGATACATATTTGAATGTAT
TTAGAAAAATAAACAAATAGGGGTTCCGCGCACATTTCCCCGAAAAGTGCCACCTGACGTC
P2Hdac9_FLAG_ENPPlopt_LINK_hAlb_pcDNA3.1 (+)
(SEQ ID NO: 90)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTATAAATGTTTTGTAGAATAAAAAAAAAAAAAGTTCTTCAAAAGAAATCTCAAATCTCC
AAATGGAAACAGGTAAAAGTGGAGCTCCCCTGGTTCCACGGAGAACCTTTTTTGAGGAAACTTAGGCAACTCG
CAGGTACCTTATGTCATGAGACAGAGTTTGAAAACTACAATTGACTATCTCTAAATTTCCTCCCAGGTCTAAA
ATGTGATGATAGTTACTACTTCAGTACATCATCCTTAAGGAAAATTATTAGGTCCACACTGTTTCTATCCTTT
GAATTTTACACATAAATTTTGTAATCAAAAGTTTATTTGTAATATCAGATGGAATCAGATAATTGCTTTTTGT
TTTTTCCACTGACAGGAACATAAGATTTTGTTGTGTAGCTTAAGTCAAACGCAGTTTGGAATATATATTTTTT
AAAAATTGTAACTTACATATCCAAATACAATTTTTCAAGAAGTAGAGTATTCAGTAGAAATTAATCTGTGAAA
GAAGAGGAATTCAGCAGTGGCCTATTTGATGAATGATTTAACGTGCTTATTTCTTCCCTTTCATCAAAACTCT
GTGTCCCCTTGTTTGCCCCCTCTGACTTCATACTCTGGAGTTGACCAAGATCCCTCTTCCATCGGATTGTTCT
GGGAATTTTGAAATAATCTGCTTTTTCCTCTCTTTCCCCTGTTGCTTCTGATGCCTTAGAATTACATTTTCCT
CGCTGATTTAGTTTAGAAAAGAGAAAAGAGCTTCCATGACTAGTAGATTATCACTTTTGGGTTTGCTCTTGGA
AGTGACAAGATGCTAGGATCCCTCTTTGGAATGTAAAATTTATCTCTTATATAGAAAGGATATAAATGTAGCA
CCAGAGACTATAAAACTCTGATACTATCTACTGTACTGTATAGCTGAACGCCACAATGTGTCTGGTAATCTAT
TGACTATCATAAATGCTATTTCTACAGAAAAGTTAGGAGGTCCATATTTCGGGCAACCAATGTATAGCTGAAT
GCAGAACAGTCATAGTTGGGTACTAACCATATATATGATTTATCCATCAACAGGTGCATATGCTCAGAAATTC
TGTATCCATAAGAAATCAGACTACTTTCTTTTCCTTTTGCAAGTAAATTGAATTTAGCCTGAGAGGCTGAGGG
GAAATTTTCACATATAAGCCACGGTTTTGTGTTTTGTGTTTTGTTTTGTTTATAGATATAGTACTAACTGGAT
GGATGCGATAAAATTCATAGGTGGTACTAAGATACAATAGGATTTGTGAAATGGACAATTGTCTTGCATAAAT
AGCAAGTAAAAAATCAAGCCTGTCCTTCATAAAAATTTTATTCTGGGGTGTCTCTGGCTAACTAGAGAACCCA
CTGCTTACTGGCTTATCGAAATTAATACGACTCACTATAGGGAGACCCAAGCTGGCTAGCGCCACCATGAAGT
GGGTAACCTTTATTTCCCTTCTTTTTCTCTTTAGCTCGGCTTATTCCAGGGGTGTGTTTCGTCGAGACTACAA
AGACGATGACGACAAGAGCGCTGGCCTGAAGCCAAGCTGCGCCAAGGAGGTGAAGAGCTGCAAGGGCAGATGT
TTCGAACGGACCTTCGGCAACTGCAGATGCGACGCCGCCTGCGTGGAACTGGGAAACTGTTGTCTGGACTACC
AGGAGACATGTATCGAGCCTGAGCACATCTGGACATGCAACAAGTTCAGATGTGGCGAGAAAAGACTGACTCG
GAGTCTGTGCGCCTGTTCTGACGACTGCAAGGACAAGGGCGATTGCTGCATCAACTACAGCAGCGTGTGCCAG
GGCGAAAAGAGCTGGGTCGAGGAACCCTGCGAGAGCATCAACGAGCCCCAGTGCCCTGCCGGATTTGAGACAC
CTCCTACCCTGCTCTTCTCCCTGGATGGCTTCAGAGCTGAGTACCTGCATACATGGGGAGGACTGCTGCCTGT
GATCAGCAAGCTGAAGAAGTGCGGCACCTACACAAAGAACATGCGGCCTGTCTACCCTACCAAGACCTTCCCC
AACCACTACAGCATCGTGACCGGCCTGTACCCCGAGAGCCACGGCATCATCGACAACAAGATGTACGACCCCA
AAATGAACGCCAGCTTCAGCCTGAAATCTAAGGAAAAGTTCAACCCTGAGTGGTATAAGGGCGAGCCAATCTG
GGTGACAGCCAAGTACCAGGGCCTGAAGAGCGGCACCTTCTTCTGGCCTGGCAGCGACGTGGAGATCAACGGA
ATCTTCCCTGATATCTACAAGATGTATAACGGCTCCGTGCCTTTCGAGGAGCGGATCTTGGCTGTTCTTCAGT
GGCTGCAGCTGCCCAAGGACGAGAGACCCCACTTCTACACCCTGTACCTGGAAGAACCTGACAGCAGCGGCCA
CAGCTACGGCCCTGTGTCTAGCGAGGTGATCAAGGCCCTGCAAAGAGTGGATGGCATGGTGGGCATGCTGATG
GATGGACTGAAGGAACTGAACCTCCATCGTTGCCTGAACCTGATCCTGATCTCCGACCACGGCATGGAACAGG
GCAGCTGTAAAAAGTACATCTACCTGAACAAGTACCTGGGCGACGTGAAGAATATCAAGGTGATCTACGGGCC
GGCCGCCAGGCTGCGGCCTAGCGACGTGCCCGACAAGTATTACAGCTTCAACTACGAGGGCATCGCCAGAAAC
CTGTCTTGCAGAGAACCCAATCAACACTTCAAGCCTTACCTGAAGCACTTTCTGCCTAAGAGACTGCACTTCG
CCAAAAGCGACAGAATTGAACCCCTGACATTCTATCTGGACCCTCAGTGGCAGCTGGCCCTCAATCCTTCTGA
ACGCAAATACTGCGGTAGCGGATTTCACGGCTCTGATAACGTGTTTTCTAACATGCAGGCCCTGTTTGTGGGC
TACGGCCCTGGCTTCAAGCACGGAATCGAGGCTGACACCTTCGAGAACATCGAAGTTTACAATCTGATGTGCG
ACCTGCTGAATCTGACCCCAGCCCCTAACAACGGCACACACGGCTCTCTGAATCACCTGCTCAAGAACCCCGT
GTATACCCCCAAACACCCTAAAGAGGTCCATCCACTGGTGCAGTGCCCATTCACCAGAAACCCTCGGGACAAC
CTTGGCTGCAGCTGCAACCCTAGCATCCTCCCTATCGAAGATTTCCAGACCCAGTTTAACCTGACTGTCGCCG
AAGAGAAGATCATCAAGCACGAAACCCTGCCCTACGGGAGGCCCAGAGTGCTGCAGAAAGAAAACACAATCTG
TCTGCTGAGCCAGCACCAGTTTATGAGCGGCTACAGTCAGGACATCCTGATGCCTCTGTGGACCAGCTACACC
GTGGACCGGAATGACAGCTTCAGCACCGAGGACTTCAGCAACTGCCTGTATCAGGACTTCCGCATCCCTCTGT
CCCCTGTGCATAAGTGCAGCTTTTACAAGAACAACACCAAGGTGAGCTACGGGTTTCTGAGCCCACCACAACT
GAACAAAAATAGCTCTGGCATCTACTCTGAGGCGCTGCTGACCACCAACATCGTGCCAATGTACCAGAGCTTC
CAGGTGATCTGGCGGTACTTCCATGATACCCTGCTTCGGAAGTACGCCGAAGAACGGAACGGCGTGAACGTGG
TGTCCGGCCCAGTGTTCGACTTCGACTACGACGGCAGATGTGATTCCCTTGAGAATCTGAGACAGAAGCGGCG
GGTTATCAGAAACCAGGAGATCCTGATTCCTACCCACTTCTTCATCGTGCTGACATCTTGCAAGGATACCAGC
CAAACACCTCTGCACTGCGAGAACCTGGACACCCTGGCCTTCATCCTGCCTCACCGGACCGATAATAGCGAAT
CCTGTGTGCACGGCAAACACGACTCTAGCTGGGTGGAAGAGCTGCTGATGCTGCACAGAGCCCGGATCACAGA
TGTGGAACACATCACCGGCCTGTCCTTCTACCAACAGAGAAAGGAACCCGTGTCTGATATCCTGAAGCTGAAA
ACCCACCTGCCTACCTTCAGCCAGGAGGACCTGATCGTTAACGATGCACACAAGAGTGAGGTTGCTCATCGGT
TTAAAGATTTGGGAGAAGAAAATTTCAAAGCCTTGGTGTTGATTGCCTTTGCTCAGTATCTTCAGCAGTGTCC
ATTTGAAGATCATGTAAAATTAGTGAATGAAGTAACTGAATTTGCAAAAACATGTGTTGCTGATGAGTCAGCT
GAAAATTGTGACAAATCACTTCATACCCTTTTTGGAGACAAATTATGCACAGTTGCAACTCTTCGTGAAACCT
ATGGTGAAATGGCTGACTGCTGTGCAAAACAAGAACCTGAGAGAAATGAATGCTTCTTGCAACACAAAGATGA
CAACCCAAACCTCCCCCGATTGGTGAGACCAGAGGTTGATGTGATGTGCACTGCTTTTCATGACAATGAAGAG
ACATTTTTGAAAAAATACTTATATGAAATTGCCAGAAGACATCCTTACTTTTATGCCCCGGAACTCCTTTTCT
TTGCTAAAAGGTATAAAGCTGCTTTTACAGAATGTTGCCAAGCTGCTGATAAAGCTGCCTGCCTGTTGCCAAA
GCTCGATGAACTTCGGGATGAAGGGAAGGCTTCGTCTGCCAAACAGAGACTCAAGTGTGCCAGTCTCCAAAAA
TTTGGAGAAAGAGCTTTCAAAGCATGGGCAGTAGCTCGCCTGAGCCAGAGATTTCCCAAAGCTGAGTTTGCAG
AAGTTTCCAAGTTAGTGACAGATCTTACCAAAGTCCACACGGAATGCTGCCATGGAGATCTGCTTGAATGTGC
TGATGACAGGGCGGACCTTGCCAAGTATATCTGTGAAAATCAAGATTCGATCTCCAGTAAACTGAAGGAATGC
TGTGAAAAACCTCTGTTGGAAAAATCCCACTGCATTGCCGAAGTGGAAAATGATGAGATGCCTGCTGACTTGC
CTTCATTAGCTGCTGATTTTGTTGAAAGTAAGGATGTTTGCAAAAACTATGCTGAGGCAAAGGATGTCTTCCT
GGGCATGTTTTTGTATGAATATGCAAGAAGGCATCCTGATTACTCTGTCGTGCTGCTGCTGAGACTTGCCAAG
ACATATGAAACCACACTCGAGAAGTGCTGTGCCGCTGCAGATCCTCATGAATGCTATGCCAAAGTGTTCGATG
AATTTAAACCTCTTGTGGAAGAGCCTCAGAATTTAATCAAACAAAATTGTGAGCTTTTTGAGCAGCTTGGAGA
GTACAAATTCCAGAATGCGCTATTAGTTCGTTACACCAAGAAAGTACCCCAAGTGTCAACTCCAACTCTTGTA
GAGGTCTCAAGAAACCTAGGAAAAGTGGGCAGCAAATGTTGTAAACATCCTGAAGCAAAAAGAATGCCCTGTG
CAGAAGACTATCTATCCGTGGTCCTGAACCAGTTATGTGTGTTGCATGAGAAAACGCCAGTAAGTGACAGAGT
CACCAAATGCTGCACAGAATCCTTGGTGAACAGGCGACCATGCTTTTCAGCTCTGGAAGTCGATGAAACATAC
GTTCCCAAAGAGTTTAATGCTGAAACATTCACCTTCCATGCAGATATATGCACACTTTCTGAGAAGGAGAGAC
AAATCAAGAAACAAACTGCACTTGTTGAGCTCGTGAAACACAAGCCCAAGGCAACAAAAGAGCAACTGAAAGC
TGTTATGGATGATTTCGCAGCTTTTGTAGAGAAGTGCTGCAAGGCTGACGATAAGGAGACCTGCTTTGCCGAG
GAGGGTAAAAAACTTGTTGCTGCAAGTCAAGCTGCCTTAGGCTTATGAGGTACCGAGCTCGGATCCACTAGTC
CAGTGTGGTGGAATTCTGCAGATATCCAGCACAGTGGCGGCCGCTCGAGTCTAGAGGGCCCGTTTAAACCCGC
TGATCAGCCTCGACTGTGCCTTCTAGTTGCCAGCCATCTGTTGTTTGCCCCTCCCCCGTGCCTTCCTTGACCC
TGGAAGGTGCCACTCCCACTGTCCTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGGAAGACAATAGCAGGCATGCTGGG
GATGCGGTGGGCTCTATGGCTTCTGAGGCGGAAAGAACCAGCTGGGGCTCTAGGGGGTATCCCCACGCGCCCT
GTAGCGGCGCATTAAGCGCGGCGGGTGTGGTGGTTACGCGCAGCGTGACCGCTACACTTGCCAGCGCCCTAGC
GCCCGCTCCTTTCGCTTTCTTCCCTTCCTTTCTCGCCACGTTCGCCGGCTTTCCCCGTCAAGCTCTAAATCGG
GGGCTCCCTTTAGGGTTCCGATTTAGTGCTTTACGGCACCTCGACCCCAAAAAACTTGATTAGGGTGATGGTT
CACGTAGTGGGCCATCGCCCTGATAGACGGTTTTTCGCCCTTTGACGTTGGAGTCCACGTTCTTTAATAGTGG
ACTCTTGTTCCAAACTGGAACAACACTCAACCCTATCTCGGTCTATTCTTTTGATTTATAAGGGATTTTGCCG
ATTTCGGCCTATTGGTTAAAAAATGAGCTGATTTAACAAAAATTTAACGCGAATTAATTCTGTGGAATGTGTG
TCAGTTAGGGTGTGGAAAGTCCCCAGGCTCCCCAGCAGGCAGAAGTATGCAAAGCATGCATCTCAATTAGTCA
GCAACCAGGTGTGGAAAGTCCCCAGGCTCCCCAGCAGGCAGAAGTATGCAAAGCATGCATCTCAATTAGTCAG
CAACCATAGTCCCGCCCCTAACTCCGCCCATCCCGCCCCTAACTCCGCCCAGTTCCGCCCATTCTCCGCCCCA
TGGCTGACTAATTTTTTTTATTTATGCAGAGGCCGAGGCCGCCTCTGCCTCTGAGCTATTCCAGAAGTAGTGA
GGAGGCTTTTTTGGAGGCCTAGGCTTTTGCAAAAAGCTCCCGGGAGCTTGTATATCCATTTTCGGATCTGATC
AAGAGACAGGATGAGGATCGTTTCGCATGATTGAACAAGATGGATTGCACGCAGGTTCTCCGGCCGCTTGGGT
GGAGAGGCTATTCGGCTATGACTGGGCACAACAGACAATCGGCTGCTCTGATGCCGCCGTGTTCCGGCTGTCA
GCGCAGGGGCGCCCGGTTCTTTTTGTCAAGACCGACCTGTCCGGTGCCCTGAATGAACTGCAGGACGAGGCAG
CGCGGCTATCGTGGCTGGCCACGACGGGCGTTCCTTGCGCAGCTGTGCTCGACGTTGTCACTGAAGCGGGAAG
GGACTGGCTGCTATTGGGCGAAGTGCCGGGGCAGGATCTCCTGTCATCTCACCTTGCTCCTGCCGAGAAAGTA
TCCATCATGGCTGATGCAATGCGGCGGCTGCATACGCTTGATCCGGCTACCTGCCCATTCGACCACCAAGCGA
AACATCGCATCGAGCGAGCACGTACTCGGATGGAAGCCGGTCTTGTCGATCAGGATGATCTGGACGAAGAGCA
TCAGGGGCTCGCGCCAGCCGAACTGTTCGCCAGGCTCAAGGCGCGCATGCCCGACGGCGAGGATCTCGTCGTG
ACCCATGGCGATGCCTGCTTGCCGAATATCATGGTGGAAAATGGCCGCTTTTCTGGATTCATCGACTGTGGCC
GGCTGGGTGTGGCGGACCGCTATCAGGACATAGCGTTGGCTACCCGTGATATTGCTGAAGAGCTTGGCGGCGA
ATGGGCTGACCGCTTCCTCGTGCTTTACGGTATCGCCGCTCCCGATTCGCAGCGCATCGCCTTCTATCGCCTT
CTTGACGAGTTCTTCTGAGCGGGACTCTGGGGTTCGAAATGACCGACCAAGCGACGCCCAACCTGCCATCACG
AGATTTCGATTCCACCGCCGCCTTCTATGAAAGGTTGGGCTTCGGAATCGTTTTCCGGGACGCCGGCTGGATG
ATCCTCCAGCGCGGGGATCTCATGCTGGAGTTCTTCGCCCACCCCAACTTGTTTATTGCAGCTTATAATGGTT
ACAAATAAAGCAATAGCATCACAAATTTCACAAATAAAGCATTTTTTTCACTGCATTCTAGTTGTGGTTTGTC
CAAACTCATCAATGTATCTTATCATGTCTGTATACCGTCGACCTCTAGCTAGAGCTTGGCGTAATCATGGTCA
TAGCTGTTTCCTGTGTGAAATTGTTATCCGCTCACAATTCCACACAACATACGAGCCGGAAGCATAAAGTGTA
AAGCCTGGGGTGCCTAATGAGTGAGCTAACTCACATTAATTGCGTTGCGCTCACTGCCCGCTTTCCAGTCGGG
AAACCTGTCGTGCCAGCTGCATTAATGAATCGGCCAACGCGCGGGGAGAGGCGGTTTGCGTATTGGGCGCTCT
TCCGCTTCCTCGCTCACTGACTCGCTGCGCTCGGTCGTTCGGCTGCGGCGAGCGGTATCAGCTCACTCAAAGG
CGGTAATACGGTTATCCACAGAATCAGGGGATAACGCAGGAAAGAACATGTGAGCAAAAGGCCAGCAAAAGGC
CAGGAACCGTAAAAAGGCCGCGTTGCTGGCGTTTTTCCATAGGCTCCGCCCCCCTGACGAGCATCACAAAAAT
CGACGCTCAAGTCAGAGGTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCCTTCGGGAAGCGTGGC
GCTTTCTCATAGCTCACGCTGTAGGTATCTCAGTTCGGTGTAGGTCGTTCGCTCCAAGCTGGGCTGTGTGCAC
GAACCCCCCGTTCAGCCCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTAGGCGGTGCTACAGAGTT
CTTGAAGTGGTGGCCTAACTACGGCTACACTAGAAGAACAGTATTTGGTATCTGCGCTCTGCTGAAGCCAGTT
ACCTTCGGAAAAAGAGTTGGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCTTTTCTACGGGGTCTGACGC
TCAGTGGAACGAAAACTCACGTTAAGGGATTTTGGTCATGAGATTATCAAAAAGGATCTTCACCTAGATCCTT
TTAAATTAAAAATGAAGTTTTAAATCAATCTAAAGTATATATGAGTAAACTTGGTCTGACAGTTACCAATGCT
TAATCAGTGAGGCACCTATCTCAGCGATCTGTCTATTTCGTTCATCCATAGTTGCCTGACTCCCCGTCGTGTA
GATAACTACGATACGGGAGGGCTTACCATCTGGCCCCAGTGCTGCAATGATACCGCGAGACCCACGCTCACCG
GCTCCAGATTTATCAGCAATAAACCAGCCAGCCGGAAGGGCCGAGCGCAGAAGTGGTCCTGCAACTTTATCCG
CCTCCATCCAGTCTATTAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTCAGCTCCGGTTCCCAA
CGATCAAGGCGAGTTACATGATCCCCCATGTTGTGCAAAAAAGCGGTTAGCTCCTTCGGTCCTCCGATCGTTG
TCAGAAGTAAGTTGGCCGCAGTGTTATCACTCATGGTTATGGCAGCACTGCATAATTCTCTTACTGTCATGCC
ATCCGTAAGATGCTTTTCTGTGACTGGTGAGTACTCAACCAAGTCATTCTGAGAATAGTGTATGCGGCGACCG
AGTTGCTCTTGCCCGGCGTCAATACGGGATAATACCGCGCCACATAGCAGAACTTTAAAAGTGCTCATCATTG
GAAAACGTTCTTCGGGGCGAAAACTCTCAAGGATCTTACCGCTGTTGAGATCCAGTTCGATGTAACCCACTCG
TGCACCCAACTGATCTTCAGCATCTTTTACTTTCACCAGCGTTTCTGGGTGAGCAAAAACAGGAAGGCAAAAT
GCCGCAAAAAAGGGAATAAGGGCGACACGGAAATGTTGAATACTCATACTCTTCCTTTTTCAATATTATTGAA
GCATTTATCAGGGTTATTGTCTCATGAGCGGATACATATTTGAATGTATTTAGAAAAATAAACAAATAGGGGT
TCCGCGCACATTTCCCCGAAAAGTGCCACCTGACGTC
CMV_kozak_hAlb_sig_FLAG_ENPPlopt_LINK_hAlb_STOP_pcDNA3.1 (+)
(CMV_srENPP1_Albumin)

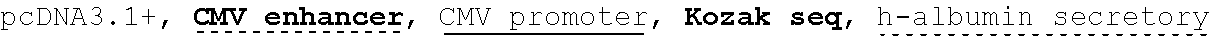

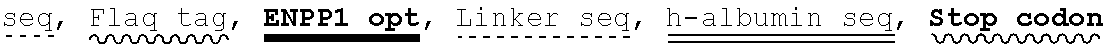

(SEQ ID NO: 91)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG

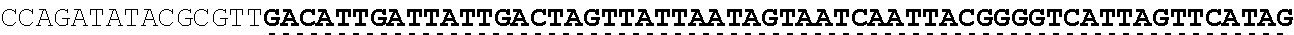

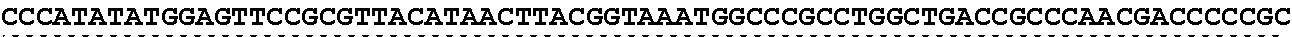

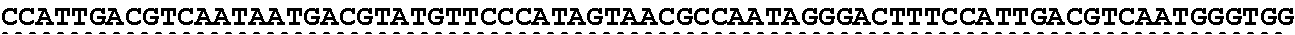

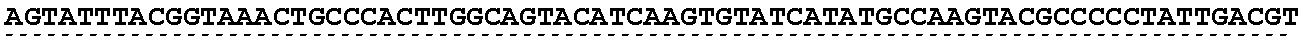

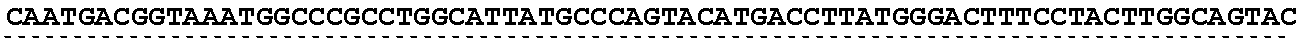

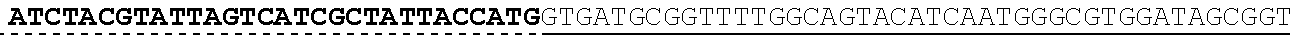

TTGACTCACGGGGATTTCCAAGTCTCCACCCCATTGACGTCAATGGGAGTTTGTTTTGGCACCAAAATCAACG
GGACTTTCCAAAATGTCGTAACAACTCCGCCCCATTGACGCAAATGGGCGGTAGGCGTGTACGGTGGGAGGTC
TATATAAGCAGAGCTCTCTGGCTAACTAGAGAACCCACTGCTTACTGGCTTATCGAAATTAATACGACTCACT

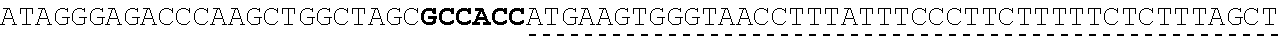

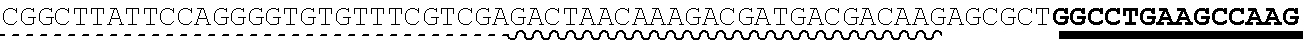

CTGCGCCAAGGAGGTGAAGAGCTGCAAGGGCAGATGTTTCGAACGGACCTTCGGCAACTGCAGATGCGACGCC
GCCTGCGTGGAACTGGGAAACTGTTGTCTGGACTACCAGGAGACATGTATCGAGCCTGAGCACATCTGGACAT
GCAACAAGTTCAGATGTGGCGAGAAAAGACTGACTCGGAGTCTGTGCGCCTGTTCTGACGACTGCAAGGACAA
GGGCGATTGCTGCATCAACTACAGCAGCGTGTGCCAGGGCGAAAAGAGCTGGGTCGAGGAACCCTGCGAGAGC
ATCAACGAGCCCCAGTGCCCTGCCGGATTTGAGACACCTCCTACCCTGCTCTTCTCCCTGGATGGCTTCAGAG
CTGAGTACCTGCATACATGGGGAGGACTGCTGCCTGTGATCAGCAAGCTGAAGAAGTGCGGCACCTACACAAA
GAACATGCGGCCTGTCTACCCTACCAAGACCTTCCCCAACCACTACAGCATCGTGACCGGCCTGTACCCCGAG
AGCCACGGCATCATCGACAACAAGATGTACGACCCCAAAATGAACGCCAGCTTCAGCCTGAAATCTAAGGAAA
AGTTCAACCCTGAGTGGTATAAGGGCGAGCCAATCTGGGTGACAGCCAAGTACCAGGGCCTGAAGAGCGGCAC
CTTCTTCTGGCCTGGCAGCGACGTGGAGATCAACGGAATCTTCCCTGATATCTACAAGATGTATAACGGCTCC
GTGCCTTTCGAGGAGCGGATCTTGGCTGTTCTTCAGTGGCTGCAGCTGCCCAAGGACGAGAGACCCCACTTCT
ACACCCTGTACCTGGAAGAACCTGACAGCAGCGGCCACAGCTACGGCCCTGTGTCTAGCGAGGTGATCAAGGC
CCTGCAAAGAGTGGATGGCATGGTGGGCATGCTGATGGATGGACTGAAGGAACTGAACCTCCATCGTTGCCTG
AACCTGATCCTGATCTCCGACCACGGCATGGAACAGGGCAGCTGTAAAAAGTACATCTACCTGAACAAGTACC
TGGGCGACGTGAAGAATATCAAGGTGATCTACGGGCCGGCCGCCAGGCTGCGGCCTAGCGACGTGCCCGACAA
TAACGTGTTTTCTAACATGCAGGCCCTGTTTGTGGGCTACGGCCCTGGCTTCAAGCACGGAATCGAGGCTGAC
ACCTTCGAGAACATCGAAGTTTACAATCTGATGTGCGACCTGCTGAATCTGACCCCAGCCCCTAACAACGGCA
CACACGGCTCTCTGAATCACCTGCTCAAGAACCCCGTGTATACCCCCAAACACCCTAAAGAGGTCCATCCACT
GGTGCAGTGCCCATTCACCAGAAACCCTCGGGACAACCTTGGCTGCAGCTGCAACCCTAGCATCCTCCCTATC
GAAGATTTCCAGACCCAGTTTAACCTGACTGTCGCCGAAGAGAAGATCATCAAGCACGAAACCCTGCCCTACG
GGAGGCCCAGAGTGCTGCAGAAAGAAAACACAATCTGTCTGCTGAGCCAGCACCAGTTTATGAGCGGCTACAG
TCAGGACATCCTGATGCCTCTGTGGACCAGCTACACCGTGGACCGGAATGACAGCTTCAGCACCGAGGACTTC
AGCAACTGCCTGTATCAGGACTTCCGCATCCCTCTGTCCCCTGTGCATAAGTGCAGCTTTTACAAGAACAACA
CCAAGGTGAGCTACGGGTTTCTGAGCCCACCACAACTGAACAAAAATAGCTCTGGCATCTACTCTGAGGCGCT
GCTGACCACCAACATCGTGCCAATGTACCAGAGCTTCCAGGTGATCTGGCGGTACTTCCATGATACCCTGCTT
CGGAAGTACGCCGAAGAACGGAACGGCGTGAACGTGGTGTCCGGCCCAGTGTTCGACTTCGACTACGACGGCA
GATGTGATTCCCTTGAGAATCTGAGACAGAAGCGGCGGGTTATCAGAAACCAGGAGATCCTGATTCCTACCCA
CTTCTTCATCGTGCTGACATCTTGCAAGGATACCAGCCAAACACCTCTGCACTGCGAGAACCTGGACACCCTG
GCCTTCATCCTGCCTCACCGGACCGATAATAGCGAATCCTGTGTGCACGGCAAACACGACTCTAGCTGGGTGG
AAGAGCTGCTGATGCTGCACAGAGCCCGGATCACAGATGTGGAACACATCACCGGCCTGTCCTTCTACCAACA

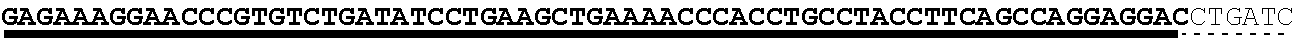

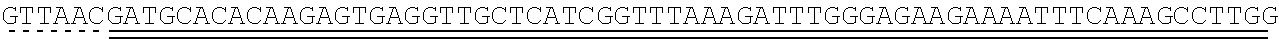

TGTTGATTGCCTTTGCTCAGTATCTTCAGCAGTGTCCATTTGAAGATCATGTAAAATTAGTGAATGAAGTAAC
TGAATTTGCAAAAACATGTGTTGCTGATGAGTCAGCTGAAAATTGTGACAAATCACTTCATACCCTTTTTGGA
GACAAATTATGCACAGTTGCAACTCTTCGTGAAACCTATGGTGAAATGGCTGACTGCTGTGCAAAACAAGAAC
CTGAGAGAAATGAATGCTTCTTGCAACACAAAGATGACAACCCAAACCTCCCCCGATTGGTGAGACCAGAGGT
TGATGTGATGTGCACTGCTTTTCATGACAATGAAGAGACATTTTTGAAAAAATACTTATATGAAATTGCCAGA
AGACATCCTTACTTTTATGCCCCGGAACTCCTTTTCTTTGCTAAAAGGTATAAAGCTGCTTTTACAGAATGTT
GCCAAGCTGCTGATAAAGCTGCCTGCCTGTTGCCAAAGCTCGATGAACTTCGGGATGAAGGGAAGGCTTCGTC
TGCCAAACAGAGACTCAAGTGTGCCAGTCTCCAAAAATTTGGAGAAAGAGCTTTCAAAGCATGGGCAGTAGCT
CGCCTGAGCCAGAGATTTCCCAAAGCTGAGTTTGCAGAAGTTTCCAAGTTAGTGACAGATCTTACCAAAGTCC
ACACGGAATGCTGCCATGGAGATCTGCTTGAATGTGCTGATGACAGGGCGGACCTTGCCAAGTATATCTGTGA
AAATCAAGATTCGATCTCCAGTAAACTGAAGGAATGCTGTGAAAAACCTCTGTTGGAAAAATCCCACTGCATT
GCCGAAGTGGAAAATGATGAGATGCCTGCTGACTTGCCTTCATTAGCTGCTGATTTTGTTGAAAGTAAGGATG
TTTGCAAAAACTATGCTGAGGCAAAGGATGTCTTCCTGGGCATGTTTTTGTATGAATATGCAAGAAGGCATCC
TGATTACTCTGTCGTGCTGCTGCTGAGACTTGCCAAGACATATGAAACCACACTCGAGAAGTGCTGTGCCGCT
GCAGATCCTCATGAATGCTATGCCAAAGTGTTCGATGAATTTAAACCTCTTGTGGAAGAGCCTCAGAATTTAA
TCAAACAAAATTGTGAGCTTTTTGAGCAGCTTGGAGAGTACAAATTCCAGAATGCGCTATTAGTTCGTTACAC
CAAGAAAGTACCCCAAGTGTCAACTCCAACTCTTGTAGAGGTCTCAAGAAACCTAGGAAAAGTGGGCAGCAAA
TGTTGTAAACATCCTGAAGCAAAAAGAATGCCCTGTGCAGAAGACTATCTATCCGTGGTCCTGAACCAGTTAT
GTGTGTTGCATGAGAAAACGCCAGTAAGTGACAGAGTCACCAAATGCTGCACAGAATCCTTGGTGAACAGGCG
ACCATGCTTTTCAGCTCTGGAAGTCGATGAAACATACGTTCCCAAAGAGTTTAATGCTGAAACATTCACCTTC
CATGCAGATATATGCACACTTTCTGAGAAGGAGAGACAAATCAAGAAACAAACTGCACTTGTTGAGCTCGTGA
AACACAAGCCCAAGGCAACAAAAGAGCAACTGAAAGCTGTTATGGATGATTTCGCAGCTTTTGTAGAGAAGTG
CTGCAAGGCTGACGATAAGGAGACCTGCTTTGCCGAGGAGGGTAAAAAACTTGTTGCTGCAAGTCAAGCTGCC

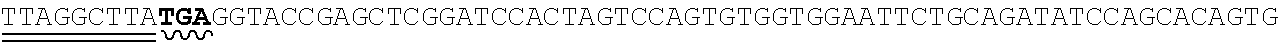

GCGGCCGCTCGAGTCTAGAGGGCCCGTTTAAACCCGCTGATCAGCCTCGACTGTGCCTTCTAGTTGCCAGCCA
TCTGTTGTTTGCCCCTCCCCCGTGCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGGGGTGGGGTGGGGCAGGACAGCAA
GGGGGAGGATTGGGAAGACAATAGCAGGCATGCTGGGGATGCGGTGGGCTCTATGGCTTCTGAGGCGGAAAGA
ACCAGCTGGGGCTCTAGGGGGTATCCCCACGCGCCCTGTAGCGGCGCATTAAGCGCGGCGGGTGTGGTGGTTA
CGCGCAGCGTGACCGCTACACTTGCCAGCGCCCTAGCGCCCGCTCCTTTCGCTTTCTTCCCTTCCTTTCTCGC
CACGTTCGCCGGCTTTCCCCGTCAAGCTCTAAATCGGGGGCTCCCTTTAGGGTTCCGATTTAGTGCTTTACGG
CACCTCGACCCCAAAAAACTTGATTAGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGGAACAACACTCAACCCTAT
CTCGGTCTATTCTTTTGATTTATAAGGGATTTTGCCGATTTCGGCCTATTGGTTAAAAAATGAGCTGATTTAA
CAAAAATTTAACGCGAATTAATTCTGTGGAATGTGTGTCAGTTAGGGTGTGGAAAGTCCCCAGGCTCCCCAGC
AGGCAGAAGTATGCAAAGCATGCATCTCAATTAGTCAGCAACCAGGTGTGGAAAGTCCCCAGGCTCCCCAGCA
GGCAGAAGTATGCAAAGCATGCATCTCAATTAGTCAGCAACCATAGTCCCGCCCCTAACTCCGCCCATCCCGC
CCCTAACTCCGCCCAGTTCCGCCCATTCTCCGCCCCATGGCTGACTAATTTTTTTTATTTATGCAGAGGCCGA
GGCCGCCTCTGCCTCTGAGCTATTCCAGAAGTAGTGAGGAGGCTTTTTTGGAGGCCTAGGCTTTTGCAAAAAG
CTCCCGGGAGCTTGTATATCCATTTTCGGATCTGATCAAGAGACAGGATGAGGATCGTTTCGCATGATTGAAC
AAGATGGATTGCACGCAGGTTCTCCGGCCGCTTGGGTGGAGAGGCTATTCGGCTATGACTGGGCACAACAGAC
AATCGGCTGCTCTGATGCCGCCGTGTTCCGGCTGTCAGCGCAGGGGCGCCCGGTTCTTTTTGTCAAGACCGAC
CTGTCCGGTGCCCTGAATGAACTGCAGGACGAGGCAGCGCGGCTATCGTGGCTGGCCACGACGGGCGTTCCTT
GCGCAGCTGTGCTCGACGTTGTCACTGAAGCGGGAAGGGACTGGCTGCTATTGGGCGAAGTGCCGGGGCAGGA
TCTCCTGTCATCTCACCTTGCTCCTGCCGAGAAAGTATCCATCATGGCTGATGCAATGCGGCGGCTGCATACG
CTTGATCCGGCTACCTGCCCATTCGACCACCAAGCGAAACATCGCATCGAGCGAGCACGTACTCGGATGGAAG
CCGGTCTTGTCGATCAGGATGATCTGGACGAAGAGCATCAGGGGCTCGCGCCAGCCGAACTGTTCGCCAGGCT
CAAGGCGCGCATGCCCGACGGCGAGGATCTCGTCGTGACCCATGGCGATGCCTGCTTGCCGAATATCATGGTG
GAAAATGGCCGCTTTTCTGGATTCATCGACTGTGGCCGGCTGGGTGTGGCGGACCGCTATCAGGACATAGCGT
TGGCTACCCGTGATATTGCTGAAGAGCTTGGCGGCGAATGGGCTGACCGCTTCCTCGTGCTTTACGGTATCGC
CGCTCCCGATTCGCAGCGCATCGCCTTCTATCGCCTTCTTGACGAGTTCTTCTGAGCGGGACTCTGGGGTTCG
AAATGACCGACCAAGCGACGCCCAACCTGCCATCACGAGATTTCGATTCCACCGCCGCCTTCTATGAAAGGTT
GGGCTTCGGAATCGTTTTCCGGGACGCCGGCTGGATGATCCTCCAGCGCGGGGATCTCATGCTGGAGTTCTTC
GCCCACCCCAACTTGTTTATTGCAGCTTATAATGGTTACAAATAAAGCAATAGCATCACAAATTTCACAAATA
AAGCATTTTTTTCACTGCATTCTAGTTGTGGTTTGTCCAAACTCATCAATGTATCTTATCATGTCTGTATACC
GTCGACCTCTAGCTAGAGCTTGGCGTAATCATGGTCATAGCTGTTTCCTGTGTGAAATTGTTATCCGCTCACA
ATTCCACACAACATACGAGCCGGAAGCATAAAGTGTAAAGCCTGGGGTGCCTAATGAGTGAGCTAACTCACAT
TAATTGCGTTGCGCTCACTGCCCGCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGCTCACTGACTCGCTGCGCTCGGTC
GTTCGGCTGCGGCGAGCGGTATCAGCTCACTCAAAGGCGGTAATACGGTTATCCACAGAATCAGGGGATAACG
CAGGAAAGAACATGTGAGCAAAAGGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGTAGGTATCTCAGTTC
GGTGTAGGTCGTTCGCTCCAAGCTGGGCTGTGTGCACGAACCCCCCGTTCAGCCCGACCGCTGCGCCTTATCC
GGTAACTATCGTCTTGAGTCCAACCCGGTAAGACACGACTTATCGCCACTGGCAGCAGCCACTGGTAACAGGA
TTAGCAGAGCGAGGTATGTAGGCGGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAAGAGTTGGTAGCTCTTGATCCGGC
AAACAAACCACCGCTGGTAGCGGTGGTTTTTTTGTTTGCAAGCAGCAGATTACGCGCAGAAAAAAAGGATCTC
AAGAAGATCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAATCTAAAGT
ATATATGAGTAAACTTGGTCTGACAGTTACCAATGCTTAATCAGTGAGGCACCTATCTCAGCGATCTGTCTAT
TTCGTTCATCCATAGTTGCCTGACTCCCCGTCGTGTAGATAACTACGATACGGGAGGGCTTACCATCTGGCCC
CAGTGCTGCAATGATACCGCGAGACCCACGCTCACCGGCTCCAGATTTATCAGCAATAAACCAGCCAGCCGGA
AGGGCCGAGCGCAGAAGTGGTCCTGCAACTTTATCCGCCTCCATCCAGTCTATTAATTGTTGCCGGGAAGCTA
GAGTAAGTAGTTCGCCAGTTAATAGTTTGCGCAACGTTGTTGCCATTGCTACAGGCATCGTGGTGTCACGCTC
GTCGTTTGGTATGGCTTCATTCAGCTCCGGTTCCCAACGATCAAGGCGAGTTACATGATCCCCCATGTTGTGC
AAAAAAGCGGTTAGCTCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTGACTGGTGAGTACTC
AACCAAGTCATTCTGAGAATAGTGTATGCGGCGACCGAGTTGCTCTTGCCCGGCGTCAATACGGGATAATACC
GCGCCACATAGCAGAACTTTAAAAGTGCTCATCATTGGAAAACGTTCTTCGGGGCGAAAACTCTCAAGGATCT
TACCGCTGTTGAGATCCAGTTCGATGTAACCCACTCGTGCACCCAACTGATCTTCAGCATCTTTTACTTTCAC
CAGCGTTTCTGGGTGAGCAAAAACAGGAAGGCAAAATGCCGCAAAAAAGGGAATAAGGGCGACACGGAAATGT
TGAATACTCATACTCTTCCTTTTTCAATATTATTGAAGCATTTATCAGGGTTATTGTCTCATGAGCGGATACA
TATTTGAATGTATTTAGAAAAATAAACAAATAGGGGTTCCGCGCACATTTCCCCGAAAAGTGCCACCTGACGT
C
CAG_Kozak_hAlb_sig_FLAG_ENPPlopt_LINK_hAlb_stop_pcDNA3.1 (+)

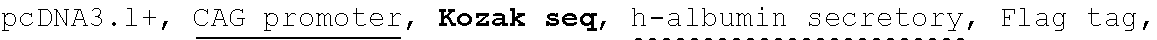

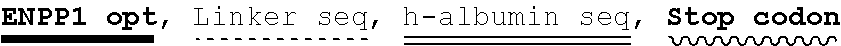

(SEQ ID NO: 92)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTGCGTTACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCATTGACGTCAATGGGTGGAG
TATTTACGGTAAACTGCCCACTTGGCAGTACATCAAGTGTATCATATGCCAAGTACGCCCCCTATTGACGTCA
ATGACGGTAAATGGCCCGCCTGGCATTATGCCCAGTACATGACCTTATGGGACTTTCCTACTTGGCAGTACAT
CTACGTATTAGTCATCGCTATTACCATGGTCGAGGTGAGCCCCACGTTCTGCTTCACTCTCCCCATCTCCCCC
CCCTCCCCACCCCCAATTTTGTATTTATTTATTTTTTAATTATTTTGTGCAGCGATGGGGGGGGGGGGGGGGG
GGGGGCGCGCGCCAGGCGGGGCGGGGCGGGGCGAGGGGCGGGGCGGGGCGAGGCGGAGAGGTGCGGCGGCAGC
CAATCAGAGCGGCGCGCTCCGAAAGTTTCCTTTTATGGCGAGGCGGCGGCGGCGGCGGCCCTATAAAAAGCGA
AGCGCGCGGCGGGCGCTCTGGCTAACTAGAGAACCCACTGCTTACTGGCTTATCGAAATTAATACGACTCACT

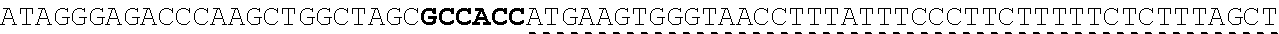

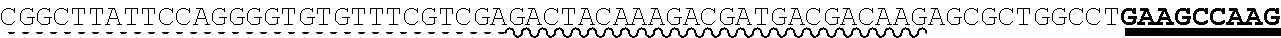

CTGCGCCAAGGAGGTGAAGAGCTGCAAGGGCAGATGTTTCGAACGGACCTTCGGCAACTGCAGATGCGACGCC
GCCTGCGTGGAACTGGGAAACTGTTGTCTGGACTACCAGGAGACATGTATCGAGCCTGAGCACATCTGGACAT
GCAACAAGTTCAGATGTGGCGAGAAAAGACTGACTCGGAGTCTGTGCGCCTGTTCTGACGACTGCAAGGACAA
GGGCGATTGCTGCATCAACTACAGCAGCGTGTGCCAGGGCGAAAAGAGCTGGGTCGAGGAACCCTGCGAGAGC
ATCAACGAGCCCCAGTGCCCTGCCGGATTTGAGACACCTCCTACCCTGCTCTTCTCCCTGGATGGCTTCAGAG
CTGAGTACCTGCATACATGGGGAGGACTGCTGCCTGTGATCAGCAAGCTGAAGAAGTGCGGCACCTACACAAA
GAACATGCGGCCTGTCTACCCTACCAAGACCTTCCCCAACCACTACAGCATCGTGACCGGCCTGTACCCCGAG
AGCCACGGCATCATCGACAACAAGATGTACGACCCCAAAATGAACGCCAGCTTCAGCCTGAAATCTAAGGAAA
AGTTCAACCCTGAGTGGTATAAGGGCGAGCCAATCTGGGTGACAGCCAAGTACCAGGGCCTGAAGAGCGGCAC
CTTCTTCTGGCCTGGCAGCGACGTGGAGATCAACGGAATCTTCCCTGATATCTACAAGATGTATAACGGCTCC
GTGCCTTTCGAGGAGCGGATCTTGGCTGTTCTTCAGTGGCTGCAGCTGCCCAAGGACGAGAGACCCCACTTCT
ACACCCTGTACCTGGAAGAACCTGACAGCAGCGGCCACAGCTACGGCCCTGTGTCTAGCGAGGTGATCAAGGC
CCTGCAAAGAGTGGATGGCATGGTGGGCATGCTGATGGATGGACTGAAGGAACTGAACCTCCATCGTTGCCTG
AACCTGATCCTGATCTCCGACCACGGCATGGAACAGGGCAGCTGTAAAAAGTACATCTACCTGAACAAGTACC
TGGGCGACGTGAAGAATATCAAGGTGATCTACGGGCCGGCCGCCAGGCTGCGGCCTAGCGACGTGCCCGACAA
GTATTACAGCTTCAACTACGAGGGCATCGCCAGAAACCTGTCTTGCAGAGAACCCAATCAACACTTCAAGCCT
TACCTGAAGCACTTTCTGCCTAAGAGACTGCACTTCGCCAAAAGCGACAGAATTGAACCCCTGACATTCTATC
TGGACCCTCAGTGGCAGCTGGCCCTCAATCCTTCTGAACGCAAATACTGCGGTAGCGGATTTCACGGCTCTGA
TAACGTGTTTTCTAACATGCAGGCCCTGTTTGTGGGCTACGGCCCTGGCTTCAAGCACGGAATCGAGGCTGAC
ACCTTCGAGAACATCGAAGTTTACAATCTGATGTGCGACCTGCTGAATCTGACCCCAGCCCCTAACAACGGCA
CACACGGCTCTCTGAATCACCTGCTCAAGAACCCCGTGTATACCCCCAAACACCCTAAAGAGGTCCATCCACT
GGTGCAGTGCCCATTCACCAGAAACCCTCGGGACAACCTTGGCTGCAGCTGCAACCCTAGCATCCTCCCTATC
GAAGATTTCCAGACCCAGTTTAACCTGACTGTCGCCGAAGAGAAGATCATCAAGCACGAAACCCTGCCCTACG
GGAGGCCCAGAGTGCTGCAGAAAGAAAACACAATCTGTCTGCTGAGCCAGCACCAGTTTATGAGCGGCTACAG
TCAGGACATCCTGATGCCTCTGTGGACCAGCTACACCGTGGACCGGAATGACAGCTTCAGCACCGAGGACTTC
AGCAACTGCCTGTATCAGGACTTCCGCATCCCTCTGTCCCCTGTGCATAAGTGCAGCTTTTACAAGAACAACA
CCAAGGTGAGCTACGGGTTTCTGAGCCCACCACAACTGAACAAAAATAGCTCTGGCATCTACTCTGAGGCGCT
GCTGACCACCAACATCGTGCCAATGTACCAGAGCTTCCAGGTGATCTGGCGGTACTTCCATGATACCCTGCTT
CGGAAGTACGCCGAAGAACGGAACGGCGTGAACGTGGTGTCCGGCCCAGTGTTCGACTTCGACTACGACGGCA
GATGTGATTCCCTTGAGAATCTGAGACAGAAGCGGCGGGTTATCAGAAACCAGGAGATCCTGATTCCTACCCA
CTTCTTCATCGTGCTGACATCTTGCAAGGATACCAGCCAAACACCTCTGCACTGCGAGAACCTGGACACCCTG
GCCTTCATCCTGCCTCACCGGACCGATAATAGCGAATCCTGTGTGCACGGCAAACACGACTCTAGCTGGGTGG
AAGAGCTGCTGATGCTGCACAGAGCCCGGATCACAGATGTGGAACACATCACCGGCCTGTCCTTCTACCAACA

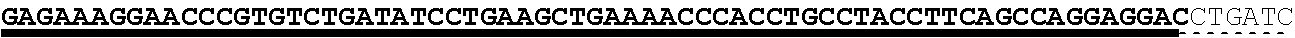

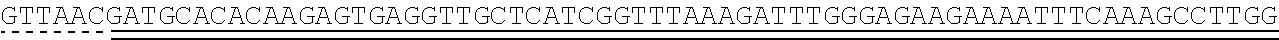

TGTTGATTGCCTTTGCTCAGTATCTTCAGCAGTGTCCATTTGAAGATCATGTAAAATTAGTGAATGAAGTAAC
TGAATTTGCAAAAACATGTGTTGCTGATGAGTCAGCTGAAAATTGTGACAAATCACTTCATACCCTTTTTGGA
GACAAATTATGCACAGTTGCAACTCTTCGTGAAACCTATGGTGAAATGGCTGACTGCTGTGCAAAACAAGAAC
CTGAGAGAAATGAATGCTTCTTGCAACACAAAGATGACAACCCAAACCTCCCCCGATTGGTGAGACCAGAGGT
TGATGTGATGTGCACTGCTTTTCATGACAATGAAGAGACATTTTTGAAAAAATACTTATATGAAATTGCCAGA
AGACATCCTTACTTTTATGCCCCGGAACTCCTTTTCTTTGCTAAAAGGTATAAAGCTGCTTTTACAGAATGTT
GCCAAGCTGCTGATAAAGCTGCCTGCCTGTTGCCAAAGCTCGATGAACTTCGGGATGAAGGGAAGGCTTCGTC
TGCCAAACAGAGACTCAAGTGTGCCAGTCTCCAAAAATTTGGAGAAAGAGCTTTCAAAGCATGGGCAGTAGCT
CGCCTGAGCCAGAGATTTCCCAAAGCTGAGTTTGCAGAAGTTTCCAAGTTAGTGACAGATCTTACCAAAGTCC
ACACGGAATGCTGCCATGGAGATCTGCTTGAATGTGCTGATGACAGGGCGGACCTTGCCAAGTATATCTGTGA
AAATCAAGATTCGATCTCCAGTAAACTGAAGGAATGCTGTGAAAAACCTCTGTTGGAAAAATCCCACTGCATT
GCCGAAGTGGAAAATGATGAGATGCCTGCTGACTTGCCTTCATTAGCTGCTGATTTTGTTGAAAGTAAGGATG
TTTGCAAAAACTATGCTGAGGCAAAGGATGTCTTCCTGGGCATGTTTTTGTATGAATATGCAAGAAGGCATCC
TGATTACTCTGTCGTGCTGCTGCTGAGACTTGCCAAGACATATGAAACCACACTCGAGAAGTGCTGTGCCGCT
GCAGATCCTCATGAATGCTATGCCAAAGTGTTCGATGAATTTAAACCTCTTGTGGAAGAGCCTCAGAATTTAA
TCAAACAAAATTGTGAGCTTTTTGAGCAGCTTGGAGAGTACAAATTCCAGAATGCGCTATTAGTTCGTTACAC
CAAGAAAGTACCCCAAGTGTCAACTCCAACTCTTGTAGAGGTCTCAAGAAACCTAGGAAAAGTGGGCAGCAAA
TGTTGTAAACATCCTGAAGCAAAAAGAATGCCCTGTGCAGAAGACTATCTATCCGTGGTCCTGAACCAGTTAT
GTGTGTTGCATGAGAAAACGCCAGTAAGTGACAGAGTCACCAAATGCTGCACAGAATCCTTGGTGAACAGGCG
ACCATGCTTTTCAGCTCTGGAAGTCGATGAAACATACGTTCCCAAAGAGTTTAATGCTGAAACATTCACCTTC
CATGCAGATATATGCACACTTTCTGAGAAGGAGAGACAAATCAAGAAACAAACTGCACTTGTTGAGCTCGTGA
AACACAAGCCCAAGGCAACAAAAGAGCAACTGAAAGCTGTTATGGATGATTTCGCAGCTTTTGTAGAGAAGTG
CTGCAAGGCTGACGATAAGGAGACCTGCTTTGCCGAGGAGGGTAAAAAACTTGTTGCTGCAAGTCAAGCTGCC

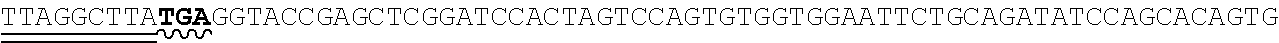

GCGGCCGCTCGAGTCTAGAGGGCCCGTTTAAACCCGCTGATCAGCCTCGACTGTGCCTTCTAGTTGCCAGCCA
TCTGTTGTTTGCCCCTCCCCCGTGCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGGGGTGGGGTGGGGCAGGACAGCAA
GGGGGAGGATTGGGAAGACAATAGCAGGCATGCTGGGGATGCGGTGGGCTCTATGGCTTCTGAGGCGGAAAGA
ACCAGCTGGGGCTCTAGGGGGTATCCCCACGCGCCCTGTAGCGGCGCATTAAGCGCGGCGGGTGTGGTGGTTA
CGCGCAGCGTGACCGCTACACTTGCCAGCGCCCTAGCGCCCGCTCCTTTCGCTTTCTTCCCTTCCTTTCTCGC
CACGTTCGCCGGCTTTCCCCGTCAAGCTCTAAATCGGGGGCTCCCTTTAGGGTTCCGATTTAGTGCTTTACGG
CACCTCGACCCCAAAAAACTTGATTAGGGTGATGGTTCACGTAGTGGGCCATCGCCCTGATAGACGGTTTTTC
GCCCTTTGACGTTGGAGTCCACGTTCTTTAATAGTGGACTCTTGTTCCAAACTGGAACAACACTCAACCCTAT
CTCGGTCTATTCTTTTGATTTATAAGGGATTTTGCCGATTTCGGCCTATTGGTTAAAAAATGAGCTGATTTAA
CAAAAATTTAACGCGAATTAATTCTGTGGAATGTGTGTCAGTTAGGGTGTGGAAAGTCCCCAGGCTCCCCAGC
AGGCAGAAGTATGCAAAGCATGCATCTCAATTAGTCAGCAACCAGGTGTGGAAAGTCCCCAGGCTCCCCAGCA
GGCAGAAGTATGCAAAGCATGCATCTCAATTAGTCAGCAACCATAGTCCCGCCCCTAACTCCGCCCATCCCGC
CCCTAACTCCGCCCAGTTCCGCCCATTCTCCGCCCCATGGCTGACTAATTTTTTTTATTTATGCAGAGGCCGA
GGCCGCCTCTGCCTCTGAGCTATTCCAGAAGTAGTGAGGAGGCTTTTTTGGAGGCCTAGGCTTTTGCAAAAAG
CTCCCGGGAGCTTGTATATCCATTTTCGGATCTGATCAAGAGACAGGATGAGGATCGTTTCGCATGATTGAAC
AAGATGGATTGCACGCAGGTTCTCCGGCCGCTTGGGTGGAGAGGCTATTCGGCTATGACTGGGCACAACAGAC
AATCGGCTGCTCTGATGCCGCCGTGTTCCGGCTGTCAGCGCAGGGGCGCCCGGTTCTTTTTGTCAAGACCGAC
CTGTCCGGTGCCCTGAATGAACTGCAGGACGAGGCAGCGCGGCTATCGTGGCTGGCCACGACGGGCGTTCCTT
GCGCAGCTGTGCTCGACGTTGTCACTGAAGCGGGAAGGGACTGGCTGCTATTGGGCGAAGTGCCGGGGCAGGA
TCTCCTGTCATCTCACCTTGCTCCTGCCGAGAAAGTATCCATCATGGCTGATGCAATGCGGCGGCTGCATACG
CTTGATCCGGCTACCTGCCCATTCGACCACCAAGCGAAACATCGCATCGAGCGAGCACGTACTCGGATGGAAG
CCGGTCTTGTCGATCAGGATGATCTGGACGAAGAGCATCAGGGGCTCGCGCCAGCCGAACTGTTCGCCAGGCT
CAAGGCGCGCATGCCCGACGGCGAGGATCTCGTCGTGACCCATGGCGATGCCTGCTTGCCGAATATCATGGTG
GAAAATGGCCGCTTTTCTGGATTCATCGACTGTGGCCGGCTGGGTGTGGCGGACCGCTATCAGGACATAGCGT
TGGCTACCCGTGATATTGCTGAAGAGCTTGGCGGCGAATGGGCTGACCGCTTCCTCGTGCTTTACGGTATCGC
CGCTCCCGATTCGCAGCGCATCGCCTTCTATCGCCTTCTTGACGAGTTCTTCTGAGCGGGACTCTGGGGTTCG
AAATGACCGACCAAGCGACGCCCAACCTGCCATCACGAGATTTCGATTCCACCGCCGCCTTCTATGAAAGGTT
GGGCTTCGGAATCGTTTTCCGGGACGCCGGCTGGATGATCCTCCAGCGCGGGGATCTCATGCTGGAGTTCTTC
GCCCACCCCAACTTGTTTATTGCAGCTTATAATGGTTACAAATAAAGCAATAGCATCACAAATTTCACAAATA
AAGCATTTTTTTCACTGCATTCTAGTTGTGGTTTGTCCAAACTCATCAATGTATCTTATCATGTCTGTATACC
GTCGACCTCTAGCTAGAGCTTGGCGTAATCATGGTCATAGCTGTTTCCTGTGTGAAATTGTTATCCGCTCACA
ATTCCACACAACATACGAGCCGGAAGCATAAAGTGTAAAGCCTGGGGTGCCTAATGAGTGAGCTAACTCACAT
TAATTGCGTTGCGCTCACTGCCCGCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGCTCACTGACTCGCTGCGCTCGGTC
GTTCGGCTGCGGCGAGCGGTATCAGCTCACTCAAAGGCGGTAATACGGTTATCCACAGAATCAGGGGATAACG
CAGGAAAGAACATGTGAGCAAAAGGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCGACCCTGCCGCTTACCG
GATACCTGTCCGCCTTTCTCCCTTCGGGAAGCGTGGCGCTTTCTCATAGCTCACGCTGTAGGTATCTCAGTTC
GGTGTAGGTCGTTCGCTCCAAGCTGGGCTGTGTGCACGAACCCCCCGTTCAGCCCGACCGCTGCGCCTTATCC
GGTAACTATCGTCTTGAGTCCAACCCGGTAAGACACGACTTATCGCCACTGGCAGCAGCCACTGGTAACAGGA
TTAGCAGAGCGAGGTATGTAGGCGGTGCTACAGAGTTCTTGAAGTGGTGGCCTAACTACGGCTACACTAGAAG
AACAGTATTTGGTATCTGCGCTCTGCTGAAGCCAGTTACCTTCGGAAAAAGAGTTGGTAGCTCTTGATCCGGC
AAACAAACCACCGCTGGTAGCGGTGGTTTTTTTGTTTGCAAGCAGCAGATTACGCGCAGAAAAAAAGGATCTC
AAGAAGATCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAATCTAAAGT
ATATATGAGTAAACTTGGTCTGACAGTTACCAATGCTTAATCAGTGAGGCACCTATCTCAGCGATCTGTCTAT
TTCGTTCATCCATAGTTGCCTGACTCCCCGTCGTGTAGATAACTACGATACGGGAGGGCTTACCATCTGGCCC
CAGTGCTGCAATGATACCGCGAGACCCACGCTCACCGGCTCCAGATTTATCAGCAATAAACCAGCCAGCCGGA
AGGGCCGAGCGCAGAAGTGGTCCTGCAACTTTATCCGCCTCCATCCAGTCTATTAATTGTTGCCGGGAAGCTA
GAGTAAGTAGTTCGCCAGTTAATAGTTTGCGCAACGTTGTTGCCATTGCTACAGGCATCGTGGTGTCACGCTC
GTCGTTTGGTATGGCTTCATTCAGCTCCGGTTCCCAACGATCAAGGCGAGTTACATGATCCCCCATGTTGTGC
AAAAAAGCGGTTAGCTCCTTCGGTCCTCCGATCGTTGTCAGAAGTAAGTTGGCCGCAGTGTTATCACTCATGG
TTATGGCAGCACTGCATAATTCTCTTACTGTCATGCCATCCGTAAGATGCTTTTCTGTGACTGGTGAGTACTC
AACCAAGTCATTCTGAGAATAGTGTATGCGGCGACCGAGTTGCTCTTGCCCGGCGTCAATACGGGATAATACC
GCGCCACATAGCAGAACTTTAAAAGTGCTCATCATTGGAAAACGTTCTTCGGGGCGAAAACTCTCAAGGATCT
TACCGCTGTTGAGATCCAGTTCGATGTAACCCACTCGTGCACCCAACTGATCTTCAGCATCTTTTACTTTCAC
CAGCGTTTCTGGGTGAGCAAAAACAGGAAGGCAAAATGCCGCAAAAAAGGGAATAAGGGCGACACGGAAATGT
TGAATACTCATACTCTTCCTTTTTCAATATTATTGAAGCATTTATCAGGGTTATTGTCTCATGAGCGGATACA
TATTTGAATGTATTTAGAAAAATAAACAAATAGGGGTTCCGCGCACATTTCCCCGAAAAGTGCCACCTGACGT
C
hALB_kozak_hAlbsig_FLAG_ENPPlopt_LINK_hAlb_pcDNA3.1(+)

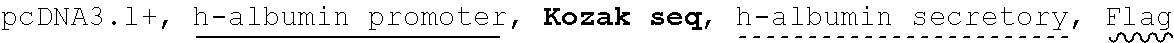

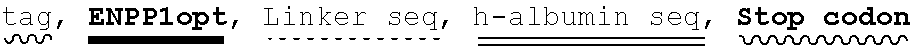

(SEQ ID NO: 93)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTAGTTCCAGATGGTAAATATACACAAGGGATTTAGTCAAACAATTTTTTGGCAAGAATA
TTATGAATTTTGTAATCGGTTGGCAGCCAATGAAATACAAAGATGAGTCTAGTTAATAATCTACAATTATTGG
TTAAAGAAGTATATTAGTGCTAATTTCCCTCCGTTTGTCCTAGCTTTTCTCTTCTGTCAACCCCACACGCCTT
TGGCACCTCTGGCTAACTAGAGAACCCACTGCTTACTGGCTTATCGAAATTAATACGACTCACTATAGGGAGA

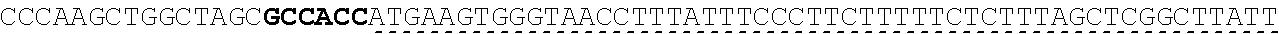

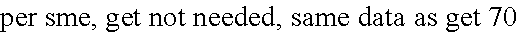

GGAGGTGAAGAGCTGCAAGGGCAGATGTTTCGAACGGACCTTCGGCAACTGCAGATGCGACGCCGCCTGCGTG
GAACTGGGAAACTGTTGTCTGGACTACCAGGAGACATGTATCGAGCCTGAGCACATCTGGACATGCAACAAGT
TCAGATGTGGCGAGAAAAGACTGACTCGGAGTCTGTGCGCCTGTTCTGACGACTGCAAGGACAAGGGCGATTG
CTGCATCAACTACAGCAGCGTGTGCCAGGGCGAAAAGAGCTGGGTCGAGGAACCCTGCGAGAGCATCAACGAG
CCCCAGTGCCCTGCCGGATTTGAGACACCTCCTACCCTGCTCTTCTCCCTGGATGGCTTCAGAGCTGAGTACC
TGCATACATGGGGAGGACTGCTGCCTGTGATCAGCAAGCTGAAGAAGTGCGGCACCTACACAAAGAACATGCG
GCCTGGCAGCGACGTGGAGATCAACGGAATCTTCCCTGATATCTACAAGATGTATAACGGCTCCGTGCCTTTC
ATCATCGACAACAAGATGTACGACCCCAAAATGAACGCCAGCTTCAGCCTGAAATCTAAGGAAAAGTTCAACC
CTGAGTGGTATAAGGGCGAGCCAATCTGGGTGACAGCCAAGTACCAGGGCCTGAAGAGCGGCACCTTCTTCTG
GCCTGGCAGCGACGTGGAGATCAACGGAATCTTCCCTGATATCTACAAGATGTATAACGGCTCCGTGCCTTTC
GAGGAGCGGATCTTGGCTGTTCTTCAGTGGCTGCAGCTGCCCAAGGACGAGAGACCCCACTTCTACACCCTGT
ACCTGGAAGAACCTGACAGCAGCGGCCACAGCTACGGCCCTGTGTCTAGCGAGGTGATCAAGGCCCTGCAAAG
AGTGGATGGCATGGTGGGCATGCTGATGGATGGACTGAAGGAACTGAACCTCCATCGTTGCCTGAACCTGATC
CTGATCTCCGACCACGGCATGGAACAGGGCAGCTGTAAAAAGTACATCTACCTGAACAAGTACCTGGGCGACG
TGAAGAATATCAAGGTGATCTACGGGCCGGCCGCCAGGCTGCGGCCTAGCGACGTGCCCGACAAGTATTACAG
CTTCAACTACGAGGGCATCGCCAGAAACCTGTCTTGCAGAGAACCCAATCAACACTTCAAGCCTTACCTGAAG
CACTTTCTGCCTAAGAGACTGCACTTCGCCAAAAGCGACAGAATTGAACCCCTGACATTCTATCTGGACCCTC
AGTGGCAGCTGGCCCTCAATCCTTCTGAACGCAAATACTGCGGTAGCGGATTTCACGGCTCTGATAACGTGTT
TTCTAACATGCAGGCCCTGTTTGTGGGCTACGGCCCTGGCTTCAAGCACGGAATCGAGGCTGACACCTTCGAG
AACATCGAAGTTTACAATCTGATGTGCGACCTGCTGAATCTGACCCCAGCCCCTAACAACGGCACACACGGCT
CTCTGAATCACCTGCTCAAGAACCCCGTGTATACCCCCAAACACCCTAAAGAGGTCCATCCACTGGTGCAGTG
CCCATTCACCAGAAACCCTCGGGACAACCTTGGCTGCAGCTGCAACCCTAGCATCCTCCCTATCGAAGATTTC
CAGACCCAGTTTAACCTGACTGTCGCCGAAGAGAAGATCATCAAGCACGAAACCCTGCCCTACGGGAGGCCCA
GAGTGCTGCAGAAAGAAAACACAATCTGTCTGCTGAGCCAGCACCAGTTTATGAGCGGCTACAGTCAGGACAT
CCTGATGCCTCTGTGGACCAGCTACACCGTGGACCGGAATGACAGCTTCAGCACCGAGGACTTCAGCAACTGC
CTGTATCAGGACTTCCGCATCCCTCTGTCCCCTGTGCATAAGTGCAGCTTTTACAAGAACAACACCAAGGTGA
GCTACGGGTTTCTGAGCCCACCACAACTGAACAAAAATAGCTCTGGCATCTACTCTGAGGCGCTGCTGACCAC
CAACATCGTGCCAATGTACCAGAGCTTCCAGGTGATCTGGCGGTACTTCCATGATACCCTGCTTCGGAAGTAC
GCCGAAGAACGGAACGGCGTGAACGTGGTGTCCGGCCCAGTGTTCGACTTCGACTACGACGGCAGATGTGATT
CCCTTGAGAATCTGAGACAGAAGCGGCGGGTTATCAGAAACCAGGAGATCCTGATTCCTACCCACTTCTTCAT
CGTGCTGACATCTTGCAAGGATACCAGCCAAACACCTCTGCACTGCGAGAACCTGGACACCCTGGCCTTCATC
CTGCCTCACCGGACCGATAATAGCGAATCCTGTGTGCACGGCAAACACGACTCTAGCTGGGTGGAAGAGCTGC
TGATGCTGCACAGAGCCCGGATCACAGATGTGGAACACATCACCGGCCTGTCCTTCTACCAACAGAGAAAGGA

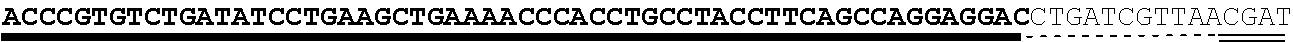

GCACACAAGAGTGAGGTTGCTCATCGGTTTAAAGATTTGGGAGAAGAAAATTTCAAAGCCTTGGTGTTGATTG
CCTTTGCTCAGTATCTTCAGCAGTGTCCATTTGAAGATCATGTAAAATTAGTGAATGAAGTAACTGAATTTGC
AAAAACATGTGTTGCTGATGAGTCAGCTGAAAATTGTGACAAATCACTTCATACCCTTTTTGGAGACAAATTA
TGCACAGTTGCAACTCTTCGTGAAACCTATGGTGAAATGGCTGACTGCTGTGCAAAACAAGAACCTGAGAGAA
ATGAATGCTTCTTGCAACACAAAGATGACAACCCAAACCTCCCCCGATTGGTGAGACCAGAGGTTGATGTGAT
GTGCACTGCTTTTCATGACAATGAAGAGACATTTTTGAAAAAATACTTATATGAAATTGCCAGAAGACATCCT
TACTTTTATGCCCCGGAACTCCTTTTCTTTGCTAAAAGGTATAAAGCTGCTTTTACAGAATGTTGCCAAGCTG
CTGATAAAGCTGCCTGCCTGTTGCCAAAGCTCGATGAACTTCGGGATGAAGGGAAGGCTTCGTCTGCCAAACA
GAGACTCAAGTGTGCCAGTCTCCAAAAATTTGGAGAAAGAGCTTTCAAAGCATGGGCAGTAGCTCGCCTGAGC
CAGAGATTTCCCAAAGCTGAGTTTGCAGAAGTTTCCAAGTTAGTGACAGATCTTACCAAAGTCCACACGGAAT
GCTGCCATGGAGATCTGCTTGAATGTGCTGATGACAGGGCGGACCTTGCCAAGTATATCTGTGAAAATCAAGA
TTCGATCTCCAGTAAACTGAAGGAATGCTGTGAAAAACCTCTGTTGGAAAAATCCCACTGCATTGCCGAAGTG
GAAAATGATGAGATGCCTGCTGACTTGCCTTCATTAGCTGCTGATTTTGTTGAAAGTAAGGATGTTTGCAAAA
ACTATGCTGAGGCAAAGGATGTCTTCCTGGGCATGTTTTTGTATGAATATGCAAGAAGGCATCCTGATTACTC
TGTCGTGCTGCTGCTGAGACTTGCCAAGACATATGAAACCACACTCGAGAAGTGCTGTGCCGCTGCAGATCCT
CATGAATGCTATGCCAAAGTGTTCGATGAATTTAAACCTCTTGTGGAAGAGCCTCAGAATTTAATCAAACAAA
ATTGTGAGCTTTTTGAGCAGCTTGGAGAGTACAAATTCCAGAATGCGCTATTAGTTCGTTACACCAAGAAAGT
ACCCCAAGTGTCAACTCCAACTCTTGTAGAGGTCTCAAGAAACCTAGGAAAAGTGGGCAGCAAATGTTGTAAA
CATCCTGAAGCAAAAAGAATGCCCTGTGCAGAAGACTATCTATCCGTGGTCCTGAACCAGTTATGTGTGTTGC
ATGAGAAAACGCCAGTAAGTGACAGAGTCACCAAATGCTGCACAGAATCCTTGGTGAACAGGCGACCATGCTT
TTCAGCTCTGGAAGTCGATGAAACATACGTTCCCAAAGAGTTTAATGCTGAAACATTCACCTTCCATGCAGAT
ATATGCACACTTTCTGAGAAGGAGAGACAAATCAAGAAACAAACTGCACTTGTTGAGCTCGTGAAACACAAGC
CCAAGGCAACAAAAGAGCAACTGAAAGCTGTTATGGATGATTTCGCAGCTTTTGTAGAGAAGTGCTGCAAGGC
TGACGATAAGGAGACCTGCTTTGCCGAGGAGGGTAAAAAACTTGTTGCTGCAAGTCAAGCTGCCTTAGGCTTA

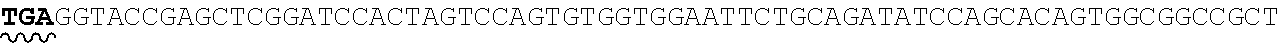

CGAGTCTAGAGGGCCCGTTTAAACCCGCTGATCAGCCTCGACTGTGCCTTCTAGTTGCCAGCCATCTGTTGTT
TGCCCCTCCCCCGTGCCTTCCTTGACCCTGGAAGGTGCCACTCCCACTGTCCTTTCCTAATAAAATGAGGAAA
TTGCATCGCATTGTCTGAGTAGGTGTCATTCTATTCTGGGGGGTGGGGTGGGGCAGGACAGCAAGGGGGAGGA
TTGGGAAGACAATAGCAGGCATGCTGGGGATGCGGTGGGCTCTATGGCTTCTGAGGCGGAAAGAACCAGCTGG
GGCTCTAGGGGGTATCCCCACGCGCCCTGTAGCGGCGCATTAAGCGCGGCGGGTGTGGTGGTTACGCGCAGCG
TGACCGCTACACTTGCCAGCGCCCTAGCGCCCGCTCCTTTCGCTTTCTTCCCTTCCTTTCTCGCCACGTTCGC
CGGCTTTCCCCGTCAAGCTCTAAATCGGGGGCTCCCTTTAGGGTTCCGATTTAGTGCTTTACGGCACCTCGAC
CCCAAAAAACTTGATTAGGGTGATGGTTCACGTAGTGGGCCATCGCCCTGATAGACGGTTTTTCGCCCTTTGA
CGTTGGAGTCCACGTTCTTTAATAGTGGACTCTTGTTCCAAACTGGAACAACACTCAACCCTATCTCGGTCTA
TTCTTTTGATTTATAAGGGATTTTGCCGATTTCGGCCTATTGGTTAAAAAATGAGCTGATTTAACAAAAATTT
AACGCGAATTAATTCTGTGGAATGTGTGTCAGTTAGGGTGTGGAAAGTCCCCAGGCTCCCCAGCAGGCAGAAG
TATGCAAAGCATGCATCTCAATTAGTCAGCAACCAGGTGTGGAAAGTCCCCAGGCTCCCCAGCAGGCAGAAGT
ATGCAAAGCATGCATCTCAATTAGTCAGCAACCATAGTCCCGCCCCTAACTCCGCCCATCCCGCCCCTAACTC
CGCCCAGTTCCGCCCATTCTCCGCCCCATGGCTGACTAATTTTTTTTATTTATGCAGAGGCCGAGGCCGCCTC
TGCCTCTGAGCTATTCCAGAAGTAGTGAGGAGGCTTTTTTGGAGGCCTAGGCTTTTGCAAAAAGCTCCCGGGA
GCTTGTATATCCATTTTCGGATCTGATCAAGAGACAGGATGAGGATCGTTTCGCATGATTGAACAAGATGGAT
TGCACGCAGGTTCTCCGGCCGCTTGGGTGGAGAGGCTATTCGGCTATGACTGGGCACAACAGACAATCGGCTG
CTCTGATGCCGCCGTGTTCCGGCTGTCAGCGCAGGGGCGCCCGGTTCTTTTTGTCAAGACCGACCTGTCCGGT
GCCCTGAATGAACTGCAGGACGAGGCAGCGCGGCTATCGTGGCTGGCCACGACGGGCGTTCCTTGCGCAGCTG
TGCTCGACGTTGTCACTGAAGCGGGAAGGGACTGGCTGCTATTGGGCGAAGTGCCGGGGCAGGATCTCCTGTC
ATCTCACCTTGCTCCTGCCGAGAAAGTATCCATCATGGCTGATGCAATGCGGCGGCTGCATACGCTTGATCCG
GCTACCTGCCCATTCGACCACCAAGCGAAACATCGCATCGAGCGAGCACGTACTCGGATGGAAGCCGGTCTTG
TCGATCAGGATGATCTGGACGAAGAGCATCAGGGGCTCGCGCCAGCCGAACTGTTCGCCAGGCTCAAGGCGCG
CATGCCCGACGGCGAGGATCTCGTCGTGACCCATGGCGATGCCTGCTTGCCGAATATCATGGTGGAAAATGGC
CGCTTTTCTGGATTCATCGACTGTGGCCGGCTGGGTGTGGCGGACCGCTATCAGGACATAGCGTTGGCTACCC
GTGATATTGCTGAAGAGCTTGGCGGCGAATGGGCTGACCGCTTCCTCGTGCTTTACGGTATCGCCGCTCCCGA
TTCGCAGCGCATCGCCTTCTATCGCCTTCTTGACGAGTTCTTCTGAGCGGGACTCTGGGGTTCGAAATGACCG
ACCAAGCGACGCCCAACCTGCCATCACGAGATTTCGATTCCACCGCCGCCTTCTATGAAAGGTTGGGCTTCGG
AATCGTTTTCCGGGACGCCGGCTGGATGATCCTCCAGCGCGGGGATCTCATGCTGGAGTTCTTCGCCCACCCC
AACTTGTTTATTGCAGCTTATAATGGTTACAAATAAAGCAATAGCATCACAAATTTCACAAATAAAGCATTTT
TTTCACTGCATTCTAGTTGTGGTTTGTCCAAACTCATCAATGTATCTTATCATGTCTGTATACCGTCGACCTC
TAGCTAGAGCTTGGCGTAATCATGGTCATAGCTGTTTCCTGTGTGAAATTGTTATCCGCTCACAATTCCACAC
AACATACGAGCCGGAAGCATAAAGTGTAAAGCCTGGGGTGCCTAATGAGTGAGCTAACTCACATTAATTGCGT
TGCGCTCACTGCCCGCTTTCCAGTCGGGAAACCTGTCGTGCCAGCTGCATTAATGAATCGGCCAACGCGCGGG
GAGAGGCGGTTTGCGTATTGGGCGCTCTTCCGCTTCCTCGCTCACTGACTCGCTGCGCTCGGTCGTTCGGCTG
CGGCGAGCGGTATCAGCTCACTCAAAGGCGGTAATACGGTTATCCACAGAATCAGGGGATAACGCAGGAAAGA
ACATGTGAGCAAAAGGCCAGCAAAAGGCCAGGAACCGTAAAAAGGCCGCGTTGCTGGCGTTTTTCCATAGGCT
CCGCCCCCCTGACGAGCATCACAAAAATCGACGCTCAAGTCAGAGGTGGCGAAACCCGACAGGACTATAAAGA
TACCAGGCGTTTCCCCCTGGAAGCTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGTAGGTATCTCAGTTCGGTGTAGGT
CGTTCGCTCCAAGCTGGGCTGTGTGCACGAACCCCCCGTTCAGCCCGACCGCTGCGCCTTATCCGGTAACTAT
CGTCTTGAGTCCAACCCGGTAAGACACGACTTATCGCCACTGGCAGCAGCCACTGGTAACAGGATTAGCAGAG
CGAGGTATGTAGGCGGTGCTACAGAGTTCTTGAAGTGGTGGCCTAACTACGGCTACACTAGAAGAACAGTATT
TGGTATCTGCGCTCTGCTGAAGCCAGTTACCTTCGGAAAAAGAGTTGGTAGCTCTTGATCCGGCAAACAAACC
ACCGCTGGTAGCGGTGGTTTTTTTGTTTGCAAGCAGCAGATTACGCGCAGAAAAAAAGGATCTCAAGAAGATC
CTTTGATCTTTTCTACGGGGTCTGACGCTCAGTGGAACGAAAACTCACGTTAAGGGATTTTGGTCATGAGATT
ATCAAAAAGGATCTTCACCTAGATCCTTTTAAATTAAAAATGAAGTTTTAAATCAATCTAAAGTATATATGAG
TAAACTTGGTCTGACAGTTACCAATGCTTAATCAGTGAGGCACCTATCTCAGCGATCTGTCTATTTCGTTCAT
CCATAGTTGCCTGACTCCCCGTCGTGTAGATAACTACGATACGGGAGGGCTTACCATCTGGCCCCAGTGCTGC
AATGATACCGCGAGACCCACGCTCACCGGCTCCAGATTTATCAGCAATAAACCAGCCAGCCGGAAGGGCCGAG
CGCAGAAGTGGTCCTGCAACTTTATCCGCCTCCATCCAGTCTATTAATTGTTGCCGGGAAGCTAGAGTAAGTA
GTTCGCCAGTTAATAGTTTGCGCAACGTTGTTGCCATTGCTACAGGCATCGTGGTGTCACGCTCGTCGTTTGG
TATGGCTTCATTCAGCTCCGGTTCCCAACGATCAAGGCGAGTTACATGATCCCCCATGTTGTGCAAAAAAGCG
GTTAGCTCCTTCGGTCCTCCGATCGTTGTCAGAAGTAAGTTGGCCGCAGTGTTATCACTCATGGTTATGGCAG
CACTGCATAATTCTCTTACTGTCATGCCATCCGTAAGATGCTTTTCTGTGACTGGTGAGTACTCAACCAAGTC
ATTCTGAGAATAGTGTATGCGGCGACCGAGTTGCTCTTGCCCGGCGTCAATACGGGATAATACCGCGCCACAT
AGCAGAACTTTAAAAGTGCTCATCATTGGAAAACGTTCTTCGGGGCGAAAACTCTCAAGGATCTTACCGCTGT
TGAGATCCAGTTCGATGTAACCCACTCGTGCACCCAACTGATCTTCAGCATCTTTTACTTTCACCAGCGTTTC
TGGGTGAGCAAAAACAGGAAGGCAAAATGCCGCAAAAAAGGGAATAAGGGCGACACGGAAATGTTGAATACTC
ATACTCTTCCTTTTTCAATATTATTGAAGCATTTATCAGGGTTATTGTCTCATGAGCGGATACATATTTGAAT
GTATTTAGAAAAATAAACAAATAGGGGTTCCGCGCACATTTCCCCGAAAAGTGCCACCTGACGTC
CMV_Kozak_hAlb sig_FLAG_ENPPlopt_LINK_hAlb_stop_mir155_pcDNA3.1(+)

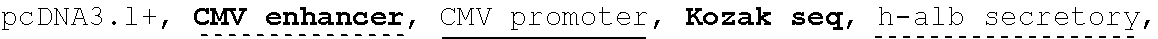

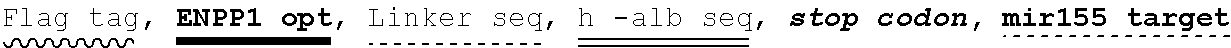

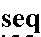

(SEQ ID NO: 94)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG

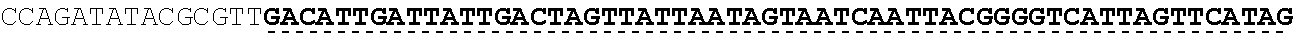

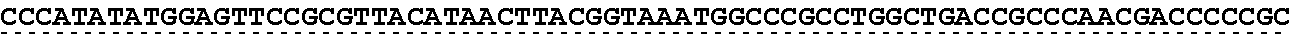

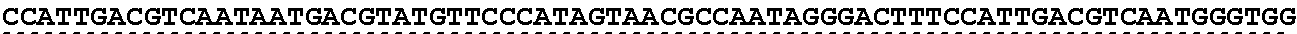

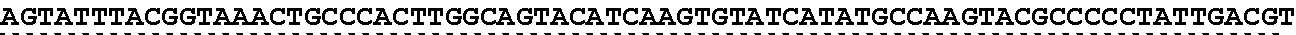

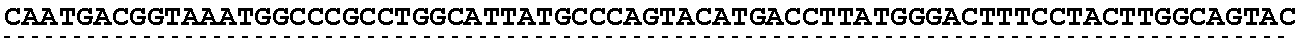

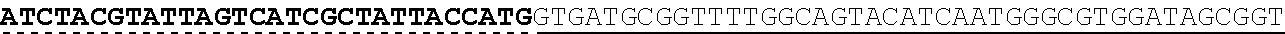

TTGACTCACGGGGATTTCCAAGTCTCCACCCCATTGACGTCAATGGGAGTTTGTTTTGGCACCAAAATCAACG
GGACTTTCCAAAATGTCGTAACAACTCCGCCCCATTGACGCAAATGGGCGGTAGGCGTGTACGGTGGGAGGTC
TATATAAGCAGAGCTCTCTGGCTAACTAGAGAACCCACTGCTTACTGGCTTATCGAAATTAATACGACTCACT

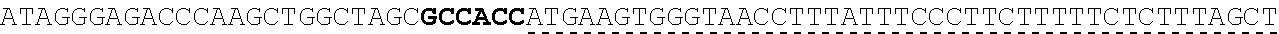

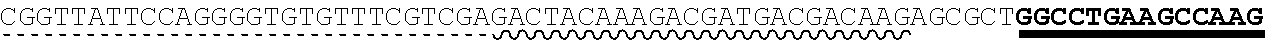

CTGCGCCAAGGAGGTGAAGAGCTGCAAGGGCAGATGTTTCGAACGGACCTTCGGCAACTGCAGATGCGACGCC
GCCTGCGTGGAACTGGGAAACTGTTGTCTGGACTACCAGGAGACATGTATCGAGCCTGAGCACATCTGGACAT
GCAACAAGTTCAGATGTGGCGAGAAAAGACTGACTCGGAGTCTGTGCGCCTGTTCTGACGACTGCAAGGACAA
GGGCGATTGCTGCATCAACTACAGCAGCGTGTGCCAGGGCGAAAAGAGCTGGGTCGAGGAACCCTGCGAGAGC
ATCAACGAGCCCCAGTGCCCTGCCGGATTTGAGACACCTCCTACCCTGCTCTTCTCCCTGGATGGCTTCAGAG
CTGAGTACCTGCATACATGGGGAGGACTGCTGCCTGTGATCAGCAAGCTGAAGAAGTGCGGCACCTACACAAA
GAACATGCGGCCTGTCTACCCTACCAAGACCTTCCCCAACCACTACAGCATCGTGACCGGCCTGTACCCCGAG
AGCCACGGCATCATCGACAACAAGATGTACGACCCCAAAATGAACGCCAGCTTCAGCCTGAAATCTAAGGAAA
AGTTCAACCCTGAGTGGTATAAGGGCGAGCCAATCTGGGTGACAGCCAAGTACCAGGGCCTGAAGAGCGGCAC
CTTCTTCTGGCCTGGCAGCGACGTGGAGATCAACGGAATCTTCCCTGATATCTACAAGATGTATAACGGCTCC
GTGCCTTTCGAGGAGCGGATCTTGGCTGTTCTTCAGTGGCTGCAGCTGCCCAAGGACGAGAGACCCCACTTCT
ACACCCTGTACCTGGAAGAACCTGACAGCAGCGGCCACAGCTACGGCCCTGTGTCTAGCGAGGTGATCAAGGC
CCTGCAAAGAGTGGATGGCATGGTGGGCATGCTGATGGATGGACTGAAGGAACTGAACCTCCATCGTTGCCTG
AACCTGATCCTGATCTCCGACCACGGCATGGAACAGGGCAGCTGTAAAAAGTACATCTACCTGAACAAGTACC
TGGGCGACGTGAAGAATATCAAGGTGATCTACGGGCCGGCCGCCAGGCTGCGGCCTAGCGACGTGCCCGACAA
GTATTACAGCTTCAACTACGAGGGCATCGCCAGAAACCTGTCTTGCAGAGAACCCAATCAACACTTCAAGCCT
TACCTGAAGCACTTTCTGCCTAAGAGACTGCACTTCGCCAAAAGCGACAGAATTGAACCCCTGACATTCTATC
TGGACCCTCAGTGGCAGCTGGCCCTCAATCCTTCTGAACGCAAATACTGCGGTAGCGGATTTCACGGCTCTGA
TAACGTGTTTTCTAACATGCAGGCCCTGTTTGTGGGCTACGGCCCTGGCTTCAAGCACGGAATCGAGGCTGAC
ACCTTCGAGAACATCGAAGTTTACAATCTGATGTGCGACCTGCTGAATCTGACCCCAGCCCCTAACAACGGCA
CACACGGCTCTCTGAATCACCTGCTCAAGAACCCCGTGTATACCCCCAAACACCCTAAAGAGGTCCATCCACT
GGTGCAGTGCCCATTCACCAGAAACCCTCGGGACAACCTTGGCTGCAGCTGCAACCCTAGCATCCTCCCTATC
GAAGATTTCCAGACCCAGTTTAACCTGACTGTCGCCGAAGAGAAGATCATCAAGCACGAAACCCTGCCCTACG
GGAGGCCCAGAGTGCTGCAGAAAGAAAACACAATCTGTCTGCTGAGCCAGCACCAGTTTATGAGCGGCTACAG
TCAGGACATCCTGATGCCTCTGTGGACCAGCTACACCGTGGACCGGAATGACAGCTTCAGCACCGAGGACTTC
AGCAACTGCCTGTATCAGGACTTCCGCATCCCTCTGTCCCCTGTGCATAAGTGCAGCTTTTACAAGAACAACA
CCAAGGTGAGCTACGGGTTTCTGAGCCCACCACAACTGAACAAAAATAGCTCTGGCATCTACTCTGAGGCGCT
GCTGACCACCAACATCGTGCCAATGTACCAGAGCTTCCAGGTGATCTGGCGGTACTTCCATGATACCCTGCTT
CGGAAGTACGCCGAAGAACGGAACGGCGTGAACGTGGTGTCCGGCCCAGTGTTCGACTTCGACTACGACGGCA
GATGTGATTCCCTTGAGAATCTGAGACAGAAGCGGCGGGTTATCAGAAACCAGGAGATCCTGATTCCTACCCA
CTTCTTCATCGTGCTGACATCTTGCAAGGATACCAGCCAAACACCTCTGCACTGCGAGAACCTGGACACCCTG
GCCTTCATCCTGCCTCACCGGACCGATAATAGCGAATCCTGTGTGCACGGCAAACACGACTCTAGCTGGGTGG
AAGAGCTGCTGATGCTGCACAGAGCCCGGATCACAGATGTGGAACACATCACCGGCCTGTCCTTCTACCAACA

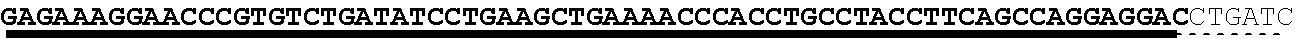

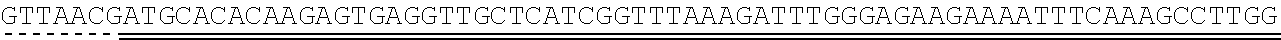

TGTTGATTGCCTTTGCTCAGTATCTTCAGCAGTGTCCATTTGAAGATCATGTAAAATTAGTGAATGAAGTAAC
TGAATTTGCAAAAACATGTGTTGCTGATGAGTCAGCTGAAAATTGTGACAAATCACTTCATACCCTTTTTGGA
GACAAATTATGCACAGTTGCAACTCTTCGTGAAACCTATGGTGAAATGGCTGACTGCTGTGCAAAACAAGAAC
CTGAGAGAAATGAATGCTTCTTGCAACACAAAGATGACAACCCAAACCTCCCCCGATTGGTGAGACCAGAGGT
TGATGTGATGTGCACTGCTTTTCATGACAATGAAGAGACATTTTTGAAAAAATACTTATATGAAATTGCCAGA
AGACATCCTTACTTTTATGCCCCGGAACTCCTTTTCTTTGCTAAAAGGTATAAAGCTGCTTTTACAGAATGTT
GCCAAGCTGCTGATAAAGCTGCCTGCCTGTTGCCAAAGCTCGATGAACTTCGGGATGAAGGGAAGGCTTCGTC
TGCCAAACAGAGACTCAAGTGTGCCAGTCTCCAAAAATTTGGAGAAAGAGCTTTCAAAGCATGGGCAGTAGCT
CGCCTGAGCCAGAGATTTCCCAAAGCTGAGTTTGCAGAAGTTTCCAAGTTAGTGACAGATCTTACCAAAGTCC
ACACGGAATGCTGCCATGGAGATCTGCTTGAATGTGCTGATGACAGGGCGGACCTTGCCAAGTATATCTGTGA
AAATCAAGATTCGATCTCCAGTAAACTGAAGGAATGCTGTGAAAAACCTCTGTTGGAAAAATCCCACTGCATT
GCCGAAGTGGAAAATGATGAGATGCCTGCTGACTTGCCTTCATTAGCTGCTGATTTTGTTGAAAGTAAGGATG
TTTGCAAAAACTATGCTGAGGCAAAGGATGTCTTCCTGGGCATGTTTTTGTATGAATATGCAAGAAGGCATCC
TGATTACTCTGTCGTGCTGCTGCTGAGACTTGCCAAGACATATGAAACCACACTCGAGAAGTGCTGTGCCGCT
GCAGATCCTCATGAATGCTATGCCAAAGTGTTCGATGAATTTAAACCTCTTGTGGAAGAGCCTCAGAATTTAA
TCAAACAAAATTGTGAGCTTTTTGAGCAGCTTGGAGAGTACAAATTCCAGAATGCGCTATTAGTTCGTTACAC
CAAGAAAGTACCCCAAGTGTCAACTCCAACTCTTGTAGAGGTCTCAAGAAACCTAGGAAAAGTGGGCAGCAAA
TGTTGTAAACATCCTGAAGCAAAAAGAATGCCCTGTGCAGAAGACTATCTATCCGTGGTCCTGAACCAGTTAT
GTGTGTTGCATGAGAAAACGCCAGTAAGTGACAGAGTCACCAAATGCTGCACAGAATCCTTGGTGAACAGGCG
ACCATGCTTTTCAGCTCTGGAAGTCGATGAAACATACGTTCCCAAAGAGTTTAATGCTGAAACATTCACCTTC
CATGCAGATATATGCACACTTTCTGAGAAGGAGAGACAAATCAAGAAACAAACTGCACTTGTTGAGCTCGTGA
AACACAAGCCCAAGGCAACAAAAGAGCAACTGAAAGCTGTTATGGATGATTTCGCAGCTTTTGTAGAGAAGTG
CTGCAAGGCTGACGATAAGGAGACCTGCTTTGCCGAGGAGGGTAAAAAACTTGTTGCTGCAAGTCAAGCTGCC

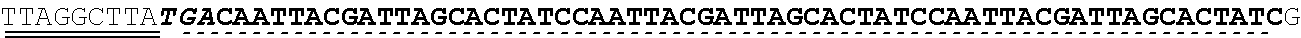

GTACCGAGCTCGGATCCACTAGTCCAGTGTGGTGGAATTCTGCAGATATCCAGCACAGTGGCGGCCGCTCGAG
TCTAGAGGGCCCGTTTAAACCCGCTGATCAGCCTCGACTGTGCCTTCTAGTTGCCAGCCATCTGTTGTTTGCC
CCTCCCCCGTGCCTTCCTTGACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAGGACAGCAAGGGGGAGGATTGG
GAAGACAATAGCAGGCATGCTGGGGATGCGGTGGGCTCTATGGCTTCTGAGGCGGAAAGAACCAGCTGGGGCT
CTAGGGGGTATCCCCACGCGCCCTGTAGCGGCGCATTAAGCGCGGCGGGTGTGGTGGTTACGCGCAGCGTGAC
CGCTACACTTGCCAGCGCCCTAGCGCCCGCTCCTTTCGCTTTCTTCCCTTCCTTTCTCGCCACGTTCGCCGGC
TTTCCCCGTCAAGCTCTAAATCGGGGGCTCCCTTTAGGGTTCCGATTTAGTGCTTTACGGCACCTCGACCCCA
AAAAACTTGATTAGGGTGATGGTTCACGTAGTGGGCCATCGCCCTGATAGACGGTTTTTCGCCCTTTGACGTT
GGAGTCCACGTTCTTTAATAGTGGACTCTTGTTCCAAACTGGAACAACACTCAACCCTATCTCGGTCTATTCT
TTTGATTTATAAGGGATTTTGCCGATTTCGGCCTATTGGTTAAAAAATGAGCTGATTTAACAAAAATTTAACG
CGAATTAATTCTGTGGAATGTGTGTCAGTTAGGGTGTGGAAAGTCCCCAGGCTCCCCAGCAGGCAGAAGTATG
CAAAGCATGCATCTCAATTAGTCAGCAACCAGGTGTGGAAAGTCCCCAGGCTCCCCAGCAGGCAGAAGTATGC
AAAGCATGCATCTCAATTAGTCAGCAACCATAGTCCCGCCCCTAACTCCGCCCATCCCGCCCCTAACTCCGCC
CAGTTCCGCCCATTCTCCGCCCCATGGCTGACTAATTTTTTTTATTTATGCAGAGGCCGAGGCCGCCTCTGCC
TCTGAGCTATTCCAGAAGTAGTGAGGAGGCTTTTTTGGAGGCCTAGGCTTTTGCAAAAAGCTCCCGGGAGCTT
GTATATCCATTTTCGGATCTGATCAAGAGACAGGATGAGGATCGTTTCGCATGATTGAACAAGATGGATTGCA
CGCAGGTTCTCCGGCCGCTTGGGTGGAGAGGCTATTCGGCTATGACTGGGCACAACAGACAATCGGCTGCTCT
GATGCCGCCGTGTTCCGGCTGTCAGCGCAGGGGCGCCCGGTTCTTTTTGTCAAGACCGACCTGTCCGGTGCCC
TGAATGAACTGCAGGACGAGGCAGCGCGGCTATCGTGGCTGGCCACGACGGGCGTTCCTTGCGCAGCTGTGCT
CGACGTTGTCACTGAAGCGGGAAGGGACTGGCTGCTATTGGGCGAAGTGCCGGGGCAGGATCTCCTGTCATCT
CACCTTGCTCCTGCCGAGAAAGTATCCATCATGGCTGATGCAATGCGGCGGCTGCATACGCTTGATCCGGCTA
CCTGCCCATTCGACCACCAAGCGAAACATCGCATCGAGCGAGCACGTACTCGGATGGAAGCCGGTCTTGTCGA
TCAGGATGATCTGGACGAAGAGCATCAGGGGCTCGCGCCAGCCGAACTGTTCGCCAGGCTCAAGGCGCGCATG
CCCGACGGCGAGGATCTCGTCGTGACCCATGGCGATGCCTGCTTGCCGAATATCATGGTGGAAAATGGCCGCT
TTTCTGGATTCATCGACTGTGGCCGGCTGGGTGTGGCGGACCGCTATCAGGACATAGCGTTGGCTACCCGTGA
TATTGCTGAAGAGCTTGGCGGCGAATGGGCTGACCGCTTCCTCGTGCTTTACGGTATCGCCGCTCCCGATTCG
CAGCGCATCGCCTTCTATCGCCTTCTTGACGAGTTCTTCTGAGCGGGACTCTGGGGTTCGAAATGACCGACCA
AGCGACGCCCAACCTGCCATCACGAGATTTCGATTCCACCGCCGCCTTCTATGAAAGGTTGGGCTTCGGAATC
GTTTTCCGGGACGCCGGCTGGATGATCCTCCAGCGCGGGGATCTCATGCTGGAGTTCTTCGCCCACCCCAACT
TGTTTATTGCAGCTTATAATGGTTACAAATAAAGCAATAGCATCACAAATTTCACAAATAAAGCATTTTTTTC
ACTGCATTCTAGTTGTGGTTTGTCCAAACTCATCAATGTATCTTATCATGTCTGTATACCGTCGACCTCTAGC
TAGAGCTTGGCGTAATCATGGTCATAGCTGTTTCCTGTGTGAAATTGTTATCCGCTCACAATTCCACACAACA
TACGAGCCGGAAGCATAAAGTGTAAAGCCTGGGGTGCCTAATGAGTGAGCTAACTCACATTAATTGCGTTGCG
CTCACTGCCCGCTTTCCAGTCGGGAAACCTGTCGTGCCAGCTGCATTAATGAATCGGCCAACGCGCGGGGAGA
GGCGGTTTGCGTATTGGGCGCTCTTCCGCTTCCTCGCTCACTGACTCGCTGCGCTCGGTCGTTCGGCTGCGGC
GAGCGGTATCAGCTCACTCAAAGGCGGTAATACGGTTATCCACAGAATCAGGGGATAACGCAGGAAAGAACAT
GTGAGCAAAAGGCCAGCAAAAGGCCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAACCCGACAGGACTATAAAGATACC
AGGCGTTTCCCCCTGGAAGCTCCCTCGTGCGCTCTCCTGTTCCGACCCTGCCGCTTACCGGATACCTGTCCGC
CTTTCTCCCTTCGGGAAGCGTGGCGCTTTCTCATAGCTCACGCTGTAGGTATCTCAGTTCGGTGTAGGTCGTT
CGCTCCAAGCTGGGCTGTGTGCACGAACCCCCCGTTCAGCCCGACCGCTGCGCCTTATCCGGTAACTATCGTC
TTGAGTCCAACCCGGTAAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAGAAGAACAGTATTTGGT
ATCTGCGCTCTGCTGAAGCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGATCTCAAGAAGATCCTTT
GATCTTTTCTACGGGGTCTGACGCTCAGTGGAACGAAAACTCACGTTAAGGGATTTTGGTCATGAGATTATCA
AAAAGGATCTTCACCTAGATCCTTTTAAATTAAAAATGAAGTTTTAAATCAATCTAAAGTATATATGAGTAAA
CTTGGTCTGACAGTTACCAATGCTTAATCAGTGAGGCACCTATCTCAGCGATCTGTCTATTTCGTTCATCCAT
AGTTGCCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGAGCGCA
GAAGTGGTCCTGCAACTTTATCCGCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGGTGTCACGCTCGTCGTTTGGTATG
GCTTCATTCAGCTCCGGTTCCCAACGATCAAGGCGAGTTACATGATCCCCCATGTTGTGCAAAAAAGCGGTTA
GCTCCTTCGGTCCTCCGATCGTTGTCAGAAGTAAGTTGGCCGCAGTGTTATCACTCATGGTTATGGCAGCACT
GCATAATTCTCTTACTGTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAATACCGCGCCACATAGCA
GAACTTTAAAAGTGCTCATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACACGGAAATGTTGAATACTCATAC
TCTTCCTTTTTCAATATTATTGAAGCATTTATCAGGGTTATTGTCTCATGAGCGGATACATATTTGAATGTAT
TTAGAAAAATAAACAAATAGGGGTTCCGCGCACATTTCCCCGAAAAGTGCCACCTGACGTC
MGP_Kozak_hAlb sig_FLAG_ENPPlopt_LINK_hAlb_stop_pcDNA3.1(+)
(SEQ ID NO: 95)

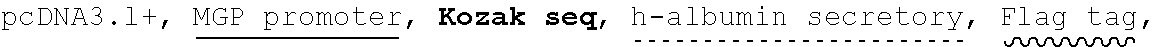

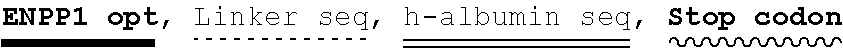

GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTAAGATTATAGTTGTCATTTGAACTTGGGGATAAAGGAGACATCTATGACTTGGCTGGA
AAAGACAGAGCTAATGTACATTGCAAAGCACATATTTATAGCAGGAAAATGGGAAGATTTCTCTTTAATTCTG
GAGATGGAGTGGGGATGGGGAGAGTAGACTACTCATTTTAAGGGTGAAACATTGGAATTCAACTTGTTTGATG
TTATATTAATTGGTGGTTAATTACTAAGCTAAGTACGTATAAAACTTTTATCTATGGCTAGCTTGTCCCCCCA
AAGTCATGCAATATAGTGAACTGGCTTTCGCACTTTAAATTATTCATTGATCATGTAATGATTCAGATGATTC
ATCTTCCAAGATGGACACTGAAACTAACACTCATAGTAGGTTGTGGTTTAAAGAGTGGAACAACCGCCAGTCT
CATTAGTGGAAATTGTGATGGTTGAATTTATCAAGGATGAACATACACGGTCTTCTTTCTGAGATTTTCTTTA
AGATTTTCGCACAGATAATCTATTTCTTAGGTTTTGGAGAGAAAACTTGAATTTTATTGATCCCTCAGAACTC
AATCTTTCAGATTTCAAAGGAGCTATTTCTTTTAATGGGGACTCTGTTAATATTTATAAAAGCTCTTCACAGG
ATGGAGGGTGGGAGGGAAACTCCATCCCAACAAGACAAAAAGAATGAAGCATGAGGCTCCACCTAGTTCATCA
CTGCTCCTTGAAATACATCAGTATTGAAAGACACATCCACCCCACCCCCAACCCAGCCCTATTGCTGTTCCAG
CTCAAGAGTCAGAGGTCCCGAAGCTGTAGCTCTTCTACAATCTGCTGCTCTGTGACTTCAAGTCTGTTGTCTG
CAAAGAAAACTATTGGGTTCCCAAGCAAGAGAGGCACATCTGGTAGGACAGATTTTGTGATTGCAAAAGAAGG
GGGAAAAAAAGAAAGAAAGAAAAGACCTCTCTATACAAGATAACCAGAGGCATCAAACTGAAATCCTCCTGTG
GAAAATAAGCTAGTACTTCTGGGCCTGATGGTGTAGTGAAAACCTGTGCTTGAGGATACATTACAGTGAAAGA
GCAAAGTGAATAGTAAGTAGCTATTACTTACCTCCTTAGGGAGGTGTGTTGTTTGTCTGTACATCCCCCACAG
CACCTAGCACAGTACCTTGCATCTCACCTGCCACTCACTAAAAAGTCTATCAAGTTAGTTAATTATCGAGACA
ACGCCCTCAGAAATGAGAGAACAGTACCCTCTTATCCTTGCTGCACTTTCCAGCACTGATACGCTGCCTAAAA
GAGGACTAGGGCACAGGTTTGAATTAATGTCACAAAACTGGATGGGCAAGTTACAACGGTGTTGATTAAGGAA
ACAGAACTCATGGTGCACCGGATATCTCCATCCTGATGAACCCTTGGAAAAATGCCAAAGATGCATATCCCCA
GGCAAATGCCTGATTAGTCTGGGATTGATAGATTGGTCTAGGATTCAGCCCTACTGGGAAGATGTCTAAATTA
TAATCAGTGTAGAAAGCGAAGTTCTCCTAGAAGAAGAGGCAAAGGTTAAAAAGAAGAAAAGAAAAGAAAGTGA
AGTCCTTTCTCCCCCAAAACCTCTCATCAATCAATCAGGGTAACAAACAGAACACTAGGGCTCTGTCTGTGGA
CCAAACCCAAAAGCCCTGCGGTCAGGGCCAGGAGGGTAGATCATGTGTTTGTGGCAACTTCCTCTGTGGGCTT
TTGCCCAGGTCTGTCCCCAAGCATACGATGGCCAAAACTTCTGCACCAGAGCAGCATCCTGTGTAACACAGTC
AGGTCCAGCAGTTAGGGAAAACTGCCCACTCAGAGTAGATAATATCTGGAAGGAATGACTGTTTGGGAAAAGT
TCCAATGCTAGTTCAGTGCCAACCCTTCCCCACCTTCTCCAGCTCTCTCCCACTGGTTCCTCCCCTCTCAACT
GCTCTGGTTCTTATAAAAACCTCACAGCCTTCCACTAACATCCCGTAGGAGCCTCTCTCCCTACTGCTGCTAC
ACAAGACCCTGAGACTGACCTGCAGGACGAAACCCTCTGGCTAACTAGAGAACCCACTGCTTACTGGCTTATC

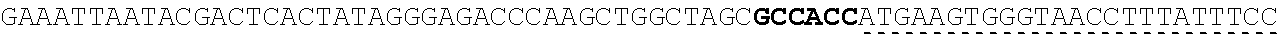

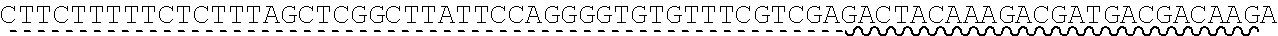

GCGCTGGCCTGAAGCCAAGCTGCGCCAAGGAGGTGAAGAGCTGCAAGGGCAGATGTTTCGAACGGACCTTCGG
CAACTGCAGATGCGACGCCGCCTGCGTGGAACTGGGAAACTGTTGTCTGGACTACCAGGAGACATGTATCGAG
CCTGAGCACATCTGGACATGCAACAAGTTCAGATGTGGCGAGAAAAGACTGACTCGGAGTCTGTGCGCCTGTT
CTGACGACTGCAAGGACAAGGGCGATTGCTGCATCAACTACAGCAGCGTGTGCCAGGGCGAAAAGAGCTGGGT
CGAGGAACCCTGCGAGAGCATCAACGAGCCCCAGTGCCCTGCCGGATTTGAGACACCTCCTACCCTGCTCTTC
TCCCTGGATGGCTTCAGAGCTGAGTACCTGCATACATGGGGAGGACTGCTGCCTGTGATCAGCAAGCTGAAGA
AGTGCGGCACCTACACAAAGAACATGCGGCCTGTCTACCCTACCAAGACCTTCCCCAACCACTACAGCATCGT
GACCGGCCTGTACCCCGAGAGCCACGGCATCATCGACAACAAGATGTACGACCCCAAAATGAACGCCAGCTTC
AGCCTGAAATCTAAGGAAAAGTTCAACCCTGAGTGGTATAAGGGCGAGCCAATCTGGGTGACAGCCAAGTACC
AGGGCCTGAAGAGCGGCACCTTCTTCTGGCCTGGCAGCGACGTGGAGATCAACGGAATCTTCCCTGATATCTA
CAAGATGTATAACGGCTCCGTGCCTTTCGAGGAGCGGATCTTGGCTGTTCTTCAGTGGCTGCAGCTGCCCAAG
GACGAGAGACCCCACTTCTACACCCTGTACCTGGAAGAACCTGACAGCAGCGGCCACAGCTACGGCCCTGTGT
CTAGCGAGGTGATCAAGGCCCTGCAAAGAGTGGATGGCATGGTGGGCATGCTGATGGATGGACTGAAGGAACT
GAACCTCCATCGTTGCCTGAACCTGATCCTGATCTCCGACCACGGCATGGAACAGGGCAGCTGTAAAAAGTAC
ATCTACCTGAACAAGTACCTGGGCGACGTGAAGAATATCAAGGTGATCTACGGGCCGGCCGCCAGGCTGCGGC
CTAGCGACGTGCCCGACAAGTATTACAGCTTCAACTACGAGGGCATCGCCAGAAACCTGTCTTGCAGAGAACC
CAATCAACACTTCAAGCCTTACCTGAAGCACTTTCTGCCTAAGAGACTGCACTTCGCCAAAAGCGACAGAATT
GAACCCCTGACATTCTATCTGGACCCTCAGTGGCAGCTGGCCCTCAATCCTTCTGAACGCAAATACTGCGGTA
GCGGATTTCACGGCTCTGATAACGTGTTTTCTAACATGCAGGCCCTGTTTGTGGGCTACGGCCCTGGCTTCAA
GCACGGAATCGAGGCTGACACCTTCGAGAACATCGAAGTTTACAATCTGATGTGCGACCTGCTGAATCTGACC
CCAGCCCCTAACAACGGCACACACGGCTCTCTGAATCACCTGCTCAAGAACCCCGTGTATACCCCCAAACACC
CTAAAGAGGTCCATCCACTGGTGCAGTGCCCATTCACCAGAAACCCTCGGGACAACCTTGGCTGCAGCTGCAA
CCCTAGCATCCTCCCTATCGAAGATTTCCAGACCCAGTTTAACCTGACTGTCGCCGAAGAGAAGATCATCAAG
CACGAAACCCTGCCCTACGGGAGGCCCAGAGTGCTGCAGAAAGAAAACACAATCTGTCTGCTGAGCCAGCACC
AGTTTATGAGCGGCTACAGTCAGGACATCCTGATGCCTCTGTGGACCAGCTACACCGTGGACCGGAATGACAG
CTTCAGCACCGAGGACTTCAGCAACTGCCTGTATCAGGACTTCCGCATCCCTCTGTCCCCTGTGCATAAGTGC
AGCTTTTACAAGAACAACACCAAGGTGAGCTACGGGTTTCTGAGCCCACCACAACTGAACAAAAATAGCTCTG
GCATCTACTCTGAGGCGCTGCTGACCACCAACATCGTGCCAATGTACCAGAGCTTCCAGGTGATCTGGCGGTA
CTTCCATGATACCCTGCTTCGGAAGTACGCCGAAGAACGGAACGGCGTGAACGTGGTGTCCGGCCCAGTGTTC
GACTTCGACTACGACGGCAGATGTGATTCCCTTGAGAATCTGAGACAGAAGCGGCGGGTTATCAGAAACCAGG
AGATCCTGATTCCTACCCACTTCTTCATCGTGCTGACATCTTGCAAGGATACCAGCCAAACACCTCTGCACTG
CGAGAACCTGGACACCCTGGCCTTCATCCTGCCTCACCGGACCGATAATAGCGAATCCTGTGTGCACGGCAAA
CACGACTCTAGCTGGGTGGAAGAGCTGCTGATGCTGCACAGAGCCCGGATCACAGATGTGGAACACATCACCG
GCCTGTCCTTCTACCAACAGAGAAAGGAACCCGTGTCTGATATCCTGAAGCTGAAAACCCACCTGCCTACCTT

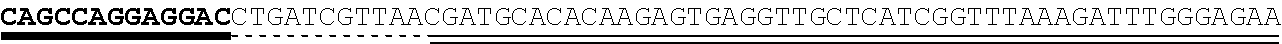

GAAAATTTCAAAGCCTTGGTGTTGATTGCCTTTGCTCAGTATCTTCAGCAGTGTCCATTTGAAGATCATGTAA
AATTAGTGAATGAAGTAACTGAATTTGCAAAAACATGTGTTGCTGATGAGTCAGCTGAAAATTGTGACAAATC
ACTTCATACCCTTTTTGGAGACAAATTATGCACAGTTGCAACTCTTCGTGAAACCTATGGTGAAATGGCTGAC
TGCTGTGCAAAACAAGAACCTGAGAGAAATGAATGCTTCTTGCAACACAAAGATGACAACCCAAACCTCCCCC
GATTGGTGAGACCAGAGGTTGATGTGATGTGCACTGCTTTTCATGACAATGAAGAGACATTTTTGAAAAAATA
CTTATATGAAATTGCCAGAAGACATCCTTACTTTTATGCCCCGGAACTCCTTTTCTTTGCTAAAAGGTATAAA
GCTGCTTTTACAGAATGTTGCCAAGCTGCTGATAAAGCTGCCTGCCTGTTGCCAAAGCTCGATGAACTTCGGG
ATGAAGGGAAGGCTTCGTCTGCCAAACAGAGACTCAAGTGTGCCAGTCTCCAAAAATTTGGAGAAAGAGCTTT
CAAAGCATGGGCAGTAGCTCGCCTGAGCCAGAGATTTCCCAAAGCTGAGTTTGCAGAAGTTTCCAAGTTAGTG
ACAGATCTTACCAAAGTCCACACGGAATGCTGCCATGGAGATCTGCTTGAATGTGCTGATGACAGGGCGGACC
TTGCCAAGTATATCTGTGAAAATCAAGATTCGATCTCCAGTAAACTGAAGGAATGCTGTGAAAAACCTCTGTT
GGAAAAATCCCACTGCATTGCCGAAGTGGAAAATGATGAGATGCCTGCTGACTTGCCTTCATTAGCTGCTGAT
TTTGTTGAAAGTAAGGATGTTTGCAAAAACTATGCTGAGGCAAAGGATGTCTTCCTGGGCATGTTTTTGTATG
AATATGCAAGAAGGCATCCTGATTACTCTGTCGTGCTGCTGCTGAGACTTGCCAAGACATATGAAACCACACT
CGAGAAGTGCTGTGCCGCTGCAGATCCTCATGAATGCTATGCCAAAGTGTTCGATGAATTTAAACCTCTTGTG
GAAGAGCCTCAGAATTTAATCAAACAAAATTGTGAGCTTTTTGAGCAGCTTGGAGAGTACAAATTCCAGAATG
CGCTATTAGTTCGTTACACCAAGAAAGTACCCCAAGTGTCAACTCCAACTCTTGTAGAGGTCTCAAGAAACCT
AGGAAAAGTGGGCAGCAAATGTTGTAAACATCCTGAAGCAAAAAGAATGCCCTGTGCAGAAGACTATCTATCC
GTGGTCCTGAACCAGTTATGTGTGTTGCATGAGAAAACGCCAGTAAGTGACAGAGTCACCAAATGCTGCACAG
AATCCTTGGTGAACAGGCGACCATGCTTTTCAGCTCTGGAAGTCGATGAAACATACGTTCCCAAAGAGTTTAA

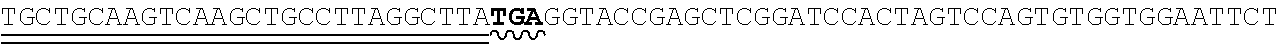

GCACTTGTTGAGCTCGTGAAACACAAGCCCAAGGCAACAAAAGAGCAACTGAAAGCTGTTATGGATGATTTCG
CAGCTTTTGTAGAGAAGTGCTGCAAGGCTGACGATAAGGAGACCTGCTTTGCCGAGGAGGGTAAAAAACTTGT
TGCTGCAAGTCAAGCTGCCTTAGGCTTATGAGGTACCGAGCTCGGATCCACTAGTCCAGTGTGGTGGAATTCT
GCAGATATCCAGCACAGTGGCGGCCGCTCGAGTCTAGAGGGCCCGTTTAAACCCGCTGATCAGCCTCGACTGT
GCCTTCTAGTTGCCAGCCATCTGTTGTTTGCCCCTCCCCCGTGCCTTCCTTGACCCTGGAAGGTGCCACTCCC
ACTGTCCTTTCCTAATAAAATGAGGAAATTGCATCGCATTGTCTGAGTAGGTGTCATTCTATTCTGGGGGGTG
GGGTGGGGCAGGACAGCAAGGGGGAGGATTGGGAAGACAATAGCAGGCATGCTGGGGATGCGGTGGGCTCTAT
GGCTTCTGAGGCGGAAAGAACCAGCTGGGGCTCTAGGGGGTATCCCCACGCGCCCTGTAGCGGCGCATTAAGC
GCGGCGGGTGTGGTGGTTACGCGCAGCGTGACCGCTACACTTGCCAGCGCCCTAGCGCCCGCTCCTTTCGCTT
TCTTCCCTTCCTTTCTCGCCACGTTCGCCGGCTTTCCCCGTCAAGCTCTAAATCGGGGGCTCCCTTTAGGGTT
CCGATTTAGTGCTTTACGGCACCTCGACCCCAAAAAACTTGATTAGGGTGATGGTTCACGTAGTGGGCCATCG
CCCTGATAGACGGTTTTTCGCCCTTTGACGTTGGAGTCCACGTTCTTTAATAGTGGACTCTTGTTCCAAACTG
GAACAACACTCAACCCTATCTCGGTCTATTCTTTTGATTTATAAGGGATTTTGCCGATTTCGGCCTATTGGTT
AAAAAATGAGCTGATTTAACAAAAATTTAACGCGAATTAATTCTGTGGAATGTGTGTCAGTTAGGGTGTGGAA
AGTCCCCAGGCTCCCCAGCAGGCAGAAGTATGCAAAGCATGCATCTCAATTAGTCAGCAACCAGGTGTGGAAA
GTCCCCAGGCTCCCCAGCAGGCAGAAGTATGCAAAGCATGCATCTCAATTAGTCAGCAACCATAGTCCCGCCC
CTAACTCCGCCCATCCCGCCCCTAACTCCGCCCAGTTCCGCCCATTCTCCGCCCCATGGCTGACTAATTTTTT
TTATTTATGCAGAGGCCGAGGCCGCCTCTGCCTCTGAGCTATTCCAGAAGTAGTGAGGAGGCTTTTTTGGAGG
CCTAGGCTTTTGCAAAAAGCTCCCGGGAGCTTGTATATCCATTTTCGGATCTGATCAAGAGACAGGATGAGGA
TCGTTTCGCATGATTGAACAAGATGGATTGCACGCAGGTTCTCCGGCCGCTTGGGTGGAGAGGCTATTCGGCT
ATGACTGGGCACAACAGACAATCGGCTGCTCTGATGCCGCCGTGTTCCGGCTGTCAGCGCAGGGGCGCCCGGT
TCTTTTTGTCAAGACCGACCTGTCCGGTGCCCTGAATGAACTGCAGGACGAGGCAGCGCGGCTATCGTGGCTG
GCCACGACGGGCGTTCCTTGCGCAGCTGTGCTCGACGTTGTCACTGAAGCGGGAAGGGACTGGCTGCTATTGG
GCGAAGTGCCGGGGCAGGATCTCCTGTCATCTCACCTTGCTCCTGCCGAGAAAGTATCCATCATGGCTGATGC
AATGCGGCGGCTGCATACGCTTGATCCGGCTACCTGCCCATTCGACCACCAAGCGAAACATCGCATCGAGCGA
GCACGTACTCGGATGGAAGCCGGTCTTGTCGATCAGGATGATCTGGACGAAGAGCATCAGGGGCTCGCGCCAG
CCGAACTGTTCGCCAGGCTCAAGGCGCGCATGCCCGACGGCGAGGATCTCGTCGTGACCCATGGCGATGCCTG
CTTGCCGAATATCATGGTGGAAAATGGCCGCTTTTCTGGATTCATCGACTGTGGCCGGCTGGGTGTGGCGGAC
CGCTATCAGGACATAGCGTTGGCTACCCGTGATATTGCTGAAGAGCTTGGCGGCGAATGGGCTGACCGCTTCC
TCGTGCTTTACGGTATCGCCGCTCCCGATTCGCAGCGCATCGCCTTCTATCGCCTTCTTGACGAGTTCTTCTG
AGCGGGACTCTGGGGTTCGAAATGACCGACCAAGCGACGCCCAACCTGCCATCACGAGATTTCGATTCCACCG
CCGCCTTCTATGAAAGGTTGGGCTTCGGAATCGTTTTCCGGGACGCCGGCTGGATGATCCTCCAGCGCGGGGA
TCTCATGCTGGAGTTCTTCGCCCACCCCAACTTGTTTATTGCAGCTTATAATGGTTACAAATAAAGCAATAGC
ATCACAAATTTCACAAATAAAGCATTTTTTTCACTGCATTCTAGTTGTGGTTTGTCCAAACTCATCAATGTAT
CTTATCATGTCTGTATACCGTCGACCTCTAGCTAGAGCTTGGCGTAATCATGGTCATAGCTGTTTCCTGTGTG
AAATTGTTATCCGCTCACAATTCCACACAACATACGAGCCGGAAGCATAAAGTGTAAAGCCTGGGGTGCCTAA
TGAGTGAGCTAACTCACATTAATTGCGTTGCGCTCACTGCCCGCTTTCCAGTCGGGAAACCTGTCGTGCCAGC
TGCATTAATGAATCGGCCAACGCGCGGGGAGAGGCGGTTTGCGTATTGGGCGCTCTTCCGCTTCCTCGCTCAC
TGACTCGCTGCGCTCGGTCGTTCGGCTGCGGCGAGCGGTATCAGCTCACTCAAAGGCGGTAATACGGTTATCC
ACAGAATCAGGGGATAACGCAGGAAAGAACATGTGAGCAAAAGGCCAGCAAAAGGCCAGGAACCGTAAAAAGG
CCGCGTTGCTGGCGTTTTTCCATAGGCTCCGCCCCCCTGACGAGCATCACAAAAATCGACGCTCAAGTCAGAG
GTGGCGAAACCCGACAGGACTATAAAGATACCAGGCGTTTCCCCCTGGAAGCTCCCTCGTGCGCTCTCCTGTT
CCGACCCTGCCGCTTACCGGATACCTGTCCGCCTTTCTCCCTTCGGGAAGCGTGGCGCTTTCTCATAGCTCAC
GCTGTAGGTATCTCAGTTCGGTGTAGGTCGTTCGCTCCAAGCTGGGCTGTGTGCACGAACCCCCCGTTCAGCC
CGACCGCTGCGCCTTATCCGGTAACTATCGTCTTGAGTCCAACCCGGTAAGACACGACTTATCGCCACTGGCA
GCAGCCACTGGTAACAGGATTAGCAGAGCGAGGTATGTAGGCGGTGCTACAGAGTTCTTGAAGTGGTGGCCTA
ACTACGGCTACACTAGAAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTGCAAGCAGCAGATTACG
CGCAGAAAAAAAGGATCTCAAGAAGATCCTTTGATCTTTTCTACGGGGTCTGACGCTCAGTGGAACGAAAACT
CACGTTAAGGGATTTTGGTCATGAGATTATCAAAAAGGATCTTCACCTAGATCCTTTTAAATTAAAAATGAAG
TTTTAAATCAATCTAAAGTATATATGAGTAAACTTGGTCTGACAGTTACCAATGCTTAATCAGTGAGGCACCT
ATCTCAGCGATCTGTCTATTTCGTTCATCCATAGTTGCCTGACTCCCCGTCGTGTAGATAACTACGATACGGG
AGGGCTTACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGCCTCCATCCAGTCTATT
AATTGTTGCCGGGAAGCTAGAGTAAGTAGTTCGCCAGTTAATAGTTTGCGCAACGTTGTTGCCATTGCTACAG
GCATCGTGGTGTCACGCTCGTCGTTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGATCGTTGTCAGAAGTAAGTTGGCC
GCAGTGTTATCACTCATGGTTATGGCAGCACTGCATAATTCTCTTACTGTCATGCCATCCGTAAGATGCTTTT
CTGTGACTGGTGAGTACTCAACCAAGTCATTCTGAGAATAGTGTATGCGGCGACCGAGTTGCTCTTGCCCGGC
GTCAATACGGGATAATACCGCGCCACATAGCAGAACTTTAAAAGTGCTCATCATTGGAAAACGTTCTTCGGGG
CGAAAACTCTCAAGGATCTTACCGCTGTTGAGATCCAGTTCGATGTAACCCACTCGTGCACCCAACTGATCTT
CAGCATCTTTTACTTTCACCAGCGTTTCTGGGTGAGCAAAAACAGGAAGGCAAAATGCCGCAAAAAAGGGAAT
AAGGGCGACACGGAAATGTTGAATACTCATACTCTTCCTTTTTCAATATTATTGAAGCATTTATCAGGGTTAT
TGTCTCATGAGCGGATACATATTTGAATGTATTTAGAAAAATAAACAAATAGGGGTTCCGCGCACATTTCCCC
GAAAAGTGCCACCTGACGTC
Prom2 (1350 bp)_hALB_kozak_hAlb sig_FLAG_ENPPlopt_LINK_hAlb_pcDNA3.1(+)

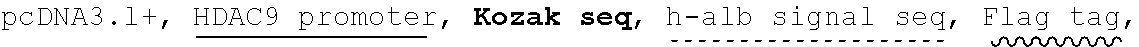

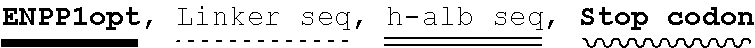

(SEQ ID NO: 96)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTATAAATGTTTTGTAGAATAAAAAAAAAAAAAGTTCTTCAAAAGAAATCTCAAATCTCC
AAATGGAAACAGGTAAAAGTGGAGCTCCCCTGGTTCCACGGAGAACCTTTTTTGAGGAAACTTAGGCAACTCG
CAGGTACCTTATGTCATGAGACAGAGTTTGAAAACTACAATTGACTATCTCTAAATTTCCTCCCAGGTCTAAA
ATGTGATGATAGTTACTACTTCAGTACATCATCCTTAAGGAAAATTATTAGGTCCACACTGTTTCTATCCTTT
GAATTTTACACATAAATTTTGTAATCAAAAGTTTATTTGTAATATCAGATGGAATCAGATAATTGCTTTTTGT
TTTTTCCACTGACAGGAACATAAGATTTTGTTGTGTAGCTTAAGTCAAACGCAGTTTGGAATATATATTTTTT
AAAAATTGTAACTTACATATCCAAATACAATTTTTCAAGAAGTAGAGTATTCAGTAGAAATTAATCTGTGAAA
GAAGAGGAATTCAGCAGTGGCCTATTTGATGAATGATTTAACGTGCTTATTTCTTCCCTTTCATCAAAACTCT
GTGTCCCCTTGTTTGCCCCCTCTGACTTCATACTCTGGAGTTGACCAAGATCCCTCTTCCATCGGATTGTTCT
GGGAATTTTGAAATAATCTGCTTTTTCCTCTCTTTCCCCTGTTGCTTCTGATGCCTTAGAATTACATTTTCCT
CGCTGATTTAGTTTAGAAAAGAGAAAAGAGCTTCCATGACTAGTAGATTATCACTTTTGGGTTTGCTCTTGGA
AGTGACAAGATGCTAGGATCCCTCTTTGGAATGTAAAATTTATCTCTTATATAGAAAGGATATAAATGTAGCA
CCAGAGACTATAAAACTCTGATACTATCTACTGTACTGTATAGCTGAACGCCACAATGTGTCTGGTAATCTAT
TGACTATCATAAATGCTATTTCTACAGAAAAGTTAGGAGGTCCATATTTCGGGCAACCAATGTATAGCTGAAT
GCAGAACAGTCATAGTTGGGTACTAACCATATATATGATTTATCCATCAACAGGTGCATATGCTCAGAAATTC
TGTATCCATAAGAAATCAGACTACTTTCTTTTCCTTTTGCAAGTAAATTGAATTTAGCCTGAGAGGCTGAGGG
GAAATTTTCACATATAAGCCACGGTTTTGTGTTTTGTGTTTTGTTTTGTTTATAGATATAGTACTAACTGGAT
GGATGCGATAAAATTCATAGGTGGTACTAAGATACAATAGGATTTGTGAAATGGACAATTGTCTTGCATAAAT
AGCAAGTAAAAAATCAAGCCTGTCCTTCATAAAAATTTTATTCTGGGGTGTCTCTGGCTAACTAGAGAACCCA

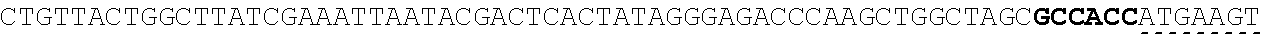

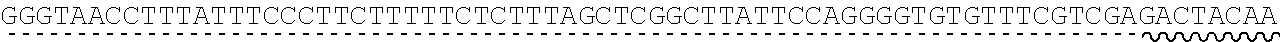

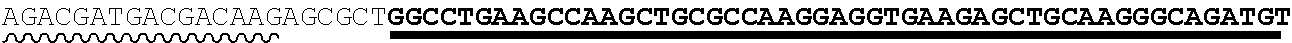

TTCGAACGGACCTTCGGCAACTGCAGATGCGACGCCGCCTGCGTGGAACTGGGAAACTGTTGTCTGGACTACC
AGGAGACATGTATCGAGCCTGAGCACATCTGGACATGCAACAAGTTCAGATGTGGCGAGAAAAGACTGACTCG
GAGTCTGTGCGCCTGTTCTGACGACTGCAAGGACAAGGGCGATTGCTGCATCAACTACAGCAGCGTGTGCCAG
GGCGAAAAGAGCTGGGTCGAGGAACCCTGCGAGAGCATCAACGACCCCCAGTGCCCTGCCGGATTTGAGACAC
CTCCTACCCTGCTCTTCTCCCTGGATGGCTTCAGAGCTGAGTACCTGCATACATGGGGAGGACTGCTGCCTGT
GATCAGCAAGCTGAAGAAGTGCGGCACCTACACAAAGAACATGCGGCCTGTCTACCCTACCAAGACCTTCCCC
AACCACTACAGCATCGTGACCGGCCTGTACCCCGAGAGCCACGGCATCATCGACAACAAGATGTACGACCCCA
AAATGAACGCCAGCTTCAGCCTGAAATCTAAGGAAAAGTTCAACCCTGAGTGGTATAAGGGCGAGCCAATCTG
GGTGACAGCCAAGTACCAGGGCCTGAAGAGCGGCACCTTCTTCTGGCCTGGCAGCGACGTGGAGATCAACGGA
ATCTTCCCTGATATCTACAAGATGTATAACGGCTCCGTGCCTTTCGAGGAGCGGATCTTGGCTGTTCTTCAGT
GGCTGCAGCTGCCCAAGGACGAGAGACCCCACTTCTACACCCTGTACCTGGAAGAACCTGACAGCAGCGGCCA
CAGCTACGGCCCTGTGTCTAGCGAGGTGATCAAGGCCCTGCAAAGAGTGGATGGCATGGTGGGCATGCTGATG
GATGGACTGAAGGAACTGAACCTCCATCGTTGCCTGAACCTGATCCTGATCTCCGACCACGGCATGGAACAGG
GCAGCTGTAAAAAGTACATCTACCTGAACAAGTACCTGGGCGACGTGAAGAATATCAAGGTGATCTACGGGCC
GGCCGCCAGGCTGCGGCCTAGCGACGTGCCCGACAAGTATTACAGCTTCAACTACGAGGGCATCGCCAGAAAC
CTGTCTTGCAGAGAACCCAATCAACACTTCAAGCCTTACCTGAAGCACTTTCTGCCTAAGAGACTGCACTTCG
CCAAAAGCGACAGAATTGAACCCCTGACATTCTATCTGGACCCTCAGTGGCAGCTGGCCCTCAATCCTTCTGA
ACGCAAATACTGCGGTAGCGGATTTCACGGCTCTGATAACGTGTTTTCTAACATGCAGGCCCTGTTTGTGGGC
TACGGCCCTGGCTTCAAGCACGGAATCGAGGCTGACACCTTCGAGAACATCGAAGTTTACAATCTGATGTGCG
ACCTGCTGAATCTGACCCCAGCCCCTAACAACGGCACACACGGCTCTCTGAATCACCTGCTCAAGAACCCCGT
GTATACCCCCAAACACCCTAAAGAGGTCCATCCACTGGTGCAGTGCCCATTCACCAGAAACCCTCGGGACAAC
CTTGGCTGCAGCTGCAACCCTAGCATCCTCCCTATCGAAGATTTCCAGACCCAGTTTAACCTGACTGTCGCCG
AAGAGAAGATCATCAAGCACGAAACCCTGCCCTACGGGAGGCCCAGAGTGCTGCAGAAAGAAAACACAATCTG
TCTGCTGAGCCAGCACCAGTTTATGAGCGGCTACAGTCAGGACATCCTGATGCCTCTGTGGACCAGCTACACC
GTGGACCGGAATGACAGCTTCAGCACCGAGGACTTCAGCAACTGCCTGTATCAGGACTTCCGCATCCCTCTGT
CCCCTGTGCATAAGTGCAGCTTTTACAAGAACAACACCAAGGTGAGCTACGGGTTTCTGAGCCCACCACAACT
GAACAAAAATAGCTCTGGCATCTACTCTGAGGCGCTGCTGACCACCAACATCGTGCCAATGTACCAGAGCTTC
CAGGTGATCTGGCGGTACTTCCATGATACCCTGCTTCGGAAGTACGCCGAAGAACGGAACGGCGTGAACGTGG
TGTCCGGCCCAGTGTTCGACTTCGACTACGACGGCAGATGTGATTCCCTTGAGAATCTGAGACAGAAGCGGCG
GGTTATCAGAAACCAGGAGATCCTGATTCCTACCCACTTCTTCATCGTGCTGACATCTTGCAAGGATACCAGC
CAAACACCTCTGCACTGCGAGAACCTGGACACCCTGGCCTTCATCCTGCCTCACCGGACCGATAATAGCGAAT
CCTGTGTGCACGGCAAACACGACTCTAGCTGGGTGGAAGAGCTGCTGATGCTGCACAGAGCCCGGATCACAGA
TGTGGAACACATCACCGGCCTGTCCTTCTACCAACAGAGAAAGGAACCCGTGTCTGATATCCTGAAGCTGAAA

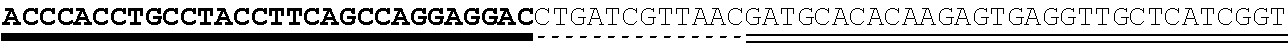

TTAAAGATTTGGGAGAAGAAAATTTCAAAGCCTTGGTGTTGATTGCCTTTGCTCAGTATCTTCAGCAGTGTCC
ATTTGAAGATCATGTAAAATTAGTGAATGAAGTAACTGAATTTGCAAAAACATGTGTTGCTGATGAGTCAGCT
GAAAATTGTGACAAATCACTTCATACCCTTTTTGGAGACAAATTATGCACAGTTGCAACTCTTCGTGAAACCT
ATGGTGAAATGGCTGACTGCTGTGCAAAACAAGAACCTGAGAGAAATGAATGCTTCTTGCAACACAAAGATGA
CAACCCAAACCTCCCCCGATTGGTGAGACCAGAGGTTGATGTGATGTGCACTGCTTTTCATGACAATGAAGAG
ACATTTTTGAAAAAATACTTATATGAAATTGCCAGAAGACATCCTTACTTTTATGCCCCGGAACTCCTTTTCT
TTGCTAAAAGGTATAAAGCTGCTTTTACAGAATGTTGCCAAGCTGCTGATAAAGCTGCCTGCCTGTTGCCAAA
GCTCGATGAACTTCGGGATGAAGGGAAGGCTTCGTCTGCCAAACAGAGACTCAAGTGTGCCAGTCTCCAAAAA
TTTGGAGAAAGAGCTTTCAAAGCATGGGCAGTAGCTCGCCTGAGCCAGAGATTTCCCAAAGCTGAGTTTGCAG
AAGTTTCCAAGTTAGTGACAGATCTTACCAAAGTCCACACGGAATGCTGCCATGGAGATCTGCTTGAATGTGC
TGATGACAGGGCGGACCTTGCCAAGTATATCTGTGAAAATCAAGATTCGATCTCCAGTAAACTGAAGGAATGC
TGTGAAAAACCTCTGTTGGAAAAATCCCACTGCATTGCCGAAGTGGAAAATGATGAGATGCCTGCTGACTTGC
CTTCATTAGCTGCTGATTTTGTTGAAAGTAAGGATGTTTGCAAAAACTATGCTGAGGCAAAGGATGTCTTCCT
GGGCATGTTTTTGTATGAATATGCAAGAAGGCATCCTGATTACTCTGTCGTGCTGCTGCTGAGACTTGCCAAG
ACATATGAAACCACACTCGAGAAGTGCTGTGCCGCTGCAGATCCTCATGAATGCTATGCCAAAGTGTTCGATG
AATTTAAACCTCTTGTGGAAGAGCCTCAGAATTTAATCAAACAAAATTGTGAGCTTTTTGAGCAGCTTGGAGA
GTACAAATTCCAGAATGCGCTATTAGTTCGTTACACCAAGAAAGTACCCCAAGTGTCAACTCCAACTCTTGTA
GAGGTCTCAAGAAACCTAGGAAAAGTGGGCAGCAAATGTTGTAAACATCCTGAAGCAAAAAGAATGCCCTGTG
CAGAAGACTATCTATCCGTGGTCCTGAACCAGTTATGTGTGTTGCATGAGAAAACGCCAGTAAGTGACAGAGT
CACCAAATGCTGCACAGAATCCTTGGTGAACAGGCGACCATGCTTTTCAGCTCTGGAAGTCGATGAAACATAC
GTTCCCAAAGAGTTTAATGCTGAAACATTCACCTTCCATGCAGATATATGCACACTTTCTGAGAAGGAGAGAC
AAATCAAGAAACAAACTGCACTTGTTGAGCTCGTGAAACACAAGCCCAAGGCAACAAAAGAGCAACTGAAAGC
TGTTATGGATGATTTCGCAGCTTTTGTAGAGAAGTGCTGCAAGGCTGACGATAAGGAGACCTGCTTTGCCGAG

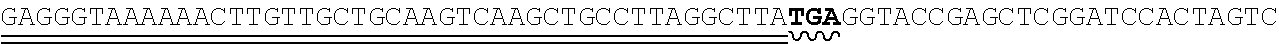

CAGTGTGGTGGAATTCTGCAGATATCCAGCACAGTGGCGGCCGCTCGAGTCTAGAGGGCCCGTTTAAACCCGC
TGATCAGCCTCGACTGTGCCTTCTAGTTGCCAGCCATCTGTTGTTTGCCCCTCCCCCGTGCCTTCCTTGACCC
TGGAAGGTGCCACTCCCACTGTCCTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGGAAGACAATAGCAGGCATGCTGGG
GATGCGGTGGGCTCTATGGCTTCTGAGGCGGAAAGAACCAGCTGGGGCTCTAGGGGGTATCCCCACGCGCCCT
GTAGCGGCGCATTAAGCGCGGCGGGTGTGGTGGTTACGCGCAGCGTGACCGCTACACTTGCCAGCGCCCTAGC
GCCCGCTCCTTTCGCTTTCTTCCCTTCCTTTCTCGCCACGTTCGCCGGCTTTCCCCGTCAAGCTCTAAATCGG
GGGCTCCCTTTAGGGTTCCGATTTAGTGCTTTACGGCACCTCGACCCCAAAAAACTTGATTAGGGTGATGGTT
CACGTAGTGGGCCATCGCCCTGATAGACGGTTTTTCGCCCTTTGACGTTGGAGTCCACGTTCTTTAATAGTGG
ACTCTTGTTCCAAACTGGAACAACACTCAACCCTATCTCGGTCTATTCTTTTGATTTATAAGGGATTTTGCCG
ATTTCGGCCTATTGGTTAAAAAATGAGCTGATTTAACAAAAATTTAACGCGAATTAATTCTGTGGAATGTGTG
TCAGTTAGGGTGTGGAAAGTCCCCAGGCTCCCCAGCAGGCAGAAGTATGCAAAGCATGCATCTCAATTAGTCA
GCAACCAGGTGTGGAAAGTCCCCAGGCTCCCCAGCAGGCAGAAGTATGCAAAGCATGCATCTCAATTAGTCAG
CAACCATAGTCCCGCCCCTAACTCCGCCCATCCCGCCCCTAACTCCGCCCAGTTCCGCCCATTCTCCGCCCCA
TGGCTGACTAATTTTTTTTATTTATGCAGAGGCCGAGGCCGCCTCTGCCTCTGAGCTATTCCAGAAGTAGTGA
GGAGGCTTTTTTGGAGGCCTAGGCTTTTGCAAAAAGCTCCCGGGAGCTTGTATATCCATTTTCGGATCTGATC
AAGAGACAGGATGAGGATCGTTTCGCATGATTGAACAAGATGGATTGCACGCAGGTTCTCCGGCCGCTTGGGT
GGAGAGGCTATTCGGCTATGACTGGGCACAACAGACAATCGGCTGCTCTGATGCCGCCGTGTTCCGGCTGTCA
GCGCAGGGGCGCCCGGTTCTTTTTGTCAAGACCGACCTGTCCGGTGCCCTGAATGAACTGCAGGACGAGGCAG
CGCGGCTATCGTGGCTGGCCACGACGGGCGTTCCTTGCGCAGCTGTGCTCGACGTTGTCACTGAAGCGGGAAG
GGACTGGCTGCTATTGGGCGAAGTGCCGGGGCAGGATCTCCTGTCATCTCACCTTGCTCCTGCCGAGAAAGTA
TCCATCATGGCTGATGCAATGCGGCGGCTGCATACGCTTGATCCGGCTACCTGCCCATTCGACCACCAAGCGA
AACATCGCATCGAGCGAGCACGTACTCGGATGGAAGCCGGTCTTGTCGATCAGGATGATCTGGACGAAGAGCA
TCAGGGGCTCGCGCCAGCCGAACTGTTCGCCAGGCTCAAGGCGCGCATGCCCGACGGCGAGGATCTCGTCGTG
ACCCATGGCGATGCCTGCTTGCCGAATATCATGGTGGAAAATGGCCGCTTTTCTGGATTCATCGACTGTGGCC
GGCTGGGTGTGGCGGACCGCTATCAGGACATAGCGTTGGCTACCCGTGATATTGCTGAAGAGCTTGGCGGCGA
ATGGGCTGACCGCTTCCTCGTGCTTTACGGTATCGCCGCTCCCGATTCGCAGCGCATCGCCTTCTATCGCCTT
CTTGACGAGTTCTTCTGAGCGGGACTCTGGGGTTCGAAATGACCGACCAAGCGACGCCCAACCTGCCATCACG
AGATTTCGATTCCACCGCCGCCTTCTATGAAAGGTTGGGCTTCGGAATCGTTTTCCGGGACGCCGGCTGGATG
ATCCTCCAGCGCGGGGATCTCATGCTGGAGTTCTTCGCCCACCCCAACTTGTTTATTGCAGCTTATAATGGTT
ACAAATAAAGCAATAGCATCACAAATTTCACAAATAAAGCATTTTTTTCACTGCATTCTAGTTGTGGTTTGTC
CAAACTCATCAATGTATCTTATCATGTCTGTATACCGTCGACCTCTAGCTAGAGCTTGGCGTAATCATGGTCA
TAGCTGTTTCCTGTGTGAAATTGTTATCCGCTCACAATTCCACACAACATACGAGCCGGAAGCATAAAGTGTA
AAGCCTGGGGTGCCTAATGAGTGAGCTAACTCACATTAATTGCGTTGCGCTCACTGCCCGCTTTCCAGTCGGG
AAACCTGTCGTGCCAGCTGCATTAATGAATCGGCCAACGCGCGGGGAGAGGCGGTTTGCGTATTGGGCGCTCT
TCCGCTTCCTCGCTCACTGACTCGCTGCGCTCGGTCGTTCGGCTGCGGCGAGCGGTATCAGCTCACTCAAAGG
CGGTAATACGGTTATCCACAGAATCAGGGGATAACGCAGGAAAGAACATGTGAGCAAAAGGCCAGCAAAAGGC
CAGGAACCGTAAAAAGGCCGCGTTGCTGGCGTTTTTCCATAGGCTCCGCCCCCCTGACGAGCATCACAAAAAT
CGACGCTCAAGTCAGAGGTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCCTTCGGGAAGCGTGGC
GCTTTCTCATAGCTCACGCTGTAGGTATCTCAGTTCGGTGTAGGTCGTTCGCTCCAAGCTGGGCTGTGTGCAC
GAACCCCCCGTTCAGCCCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTAGGCGGTGCTACAGAGTT
CTTGAAGTGGTGGCCTAACTACGGCTACACTAGAAGAACAGTATTTGGTATCTGCGCTCTGCTGAAGCCAGTT
ACCTTCGGAAAAAGAGTTGGTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGATCTTTTCTACGGGGTCTGACGC
TCAGTGGAACGAAAACTCACGTTAAGGGATTTTGGTCATGAGATTATCAAAAAGGATCTTCACCTAGATCCTT
TTAAATTAAAAATGAAGTTTTAAATCAATCTAAAGTATATATGAGTAAACTTGGTCTGACAGTTACCAATGCT
TAATCAGTGAGGCACCTATCTCAGCGATCTGTCTATTTCGTTCATCCATAGTTGCCTGACTCCCCGTCGTGTA
GATAACTACGATACGGGAGGGCTTACCATCTGGCCCCAGTGCTGCAATGATACCGCGAGACCCACGCTCACCG
GCTCCAGATTTATCAGCAATAAACCAGCCAGCCGGAAGGGCCGAGCGCAGAAGTGGTCCTGCAACTTTATCCG
CCTCCATCCAGTCTATTAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTCAGCTCCGGTTCCCAA
CGATCAAGGCGAGTTACATGATCCCCCATGTTGTGCAAAAAAGCGGTTAGCTCCTTCGGTCCTCCGATCGTTG
TCAGAAGTAAGTTGGCCGCAGTGTTATCACTCATGGTTATGGCAGCACTGCATAATTCTCTTACTGTCATGCC
ATCCGTAAGATGCTTTTCTGTGACTGGTGAGTACTCAACCAAGTCATTCTGAGAATAGTGTATGCGGCGACCG
AGTTGCTCTTGCCCGGCGTCAATACGGGATAATACCGCGCCACATAGCAGAACTTTAAAAGTGCTCATCATTG
GAAAACGTTCTTCGGGGCGAAAACTCTCAAGGATCTTACCGCTGTTGAGATCCAGTTCGATGTAACCCACTCG
TGCACCCAACTGATCTTCAGCATCTTTTACTTTCACCAGCGTTTCTGGGTGAGCAAAAACAGGAAGGCAAAAT
GCCGCAAAAAAGGGAATAAGGGCGACACGGAAATGTTGAATACTCATACTCTTCCTTTTTCAATATTATTGAA
GCATTTATCAGGGTTATTGTCTCATGAGCGGATACATATTTGAATGTATTTAGAAAAATAAACAAATAGGGGT
TCCGCGCACATTTCCCCGAAAAGTGCCACCTGACGTC
MGP_Kozak_hMGP sig_FLAG_ENPPlopt_LINK_hAlb_stop_pcDNA3.1(+)
(SEQ ID NO: 97)

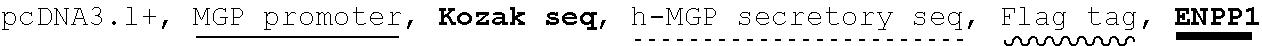

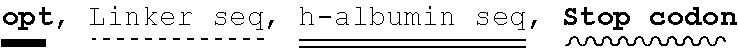

GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTAAGATTATAGTTGTCATTTGAACTTGGGGATAAAGGAGACATCTATGACTTGGCTGGA
AAAGACAGAGCTAATGTACATTGCAAAGCACATATTTATAGCAGGAAAATGGGAAGATTTCTCTTTAATTCTG
GAGATGGAGTGGGGATGGGGAGAGTAGACTACTCATTTTAAGGGTGAAACATTGGAATTCAACTTGTTTGATG
TTATATTAATTGGTGGTTAATTACTAAGCTAAGTACGTATAAAACTTTTATCTATGGCTAGCTTGTCCCCCCA
AAGTCATGCAATATAGTGAACTGGCTTTCGCACTTTAAATTATTCATTGATCATGTAATGATTCAGATGATTC
ATCTTCCAAGATGGACACTGAAACTAACACTCATAGTAGGTTGTGGTTTAAAGAGTGGAACAACCGCCAGTCT
CATTAGTGGAAATTGTGATGGTTGAATTTATCAAGGATGAACATACACGGTCTTCTTTCTGAGATTTTCTTTA
AGATTTTCGCACAGATAATCTATTTCTTAGGTTTTGGAGAGAAAACTTGAATTTTATTGATCCCTCAGAACTC
AATCTTTCAGATTTCAAAGGAGCTATTTCTTTTAATGGGGACTCTGTTAATATTTATAAAAGCTCTTCACAGG
ATGGAGGGTGGGAGGGAAACTCCATCCCAACAAGACAAAAAGAATGAAGCATGAGGCTCCACCTAGTTCATCA
CTGCTCCTTGAAATACATCAGTATTGAAAGACACATCCACCCCACCCCCAACCCAGCCCTATTGCTGTTCCAG
CTCAAGAGTCAGAGGTCCCGAAGCTGTAGCTCTTCTACAATCTGCTGCTCTGTGACTTCAAGTCTGTTGTCTG
CAAAGAAAACTATTGGGTTCCCAAGCAAGAGAGGCACATCTGGTAGGACAGATTTTGTGATTGCAAAAGAAGG
GGGAAAAAAAGAAAGAAAGAAAAGACCTCTCTATACAAGATAACCAGAGGCATCAAACTGAAATCCTCCTGTG
GAAAATAAGCTAGTACTTCTGGGCCTGATGGTGTAGTGAAAACCTGTGCTTGAGGATACATTACAGTGAAAGA
GCAAAGTGAATAGTAAGTAGCTATTACTTACCTCCTTAGGGAGGTGTGTTGTTTGTCTGTACATCCCCCACAG
CACCTAGCACAGTACCTTGCATCTCACCTGCCACTCACTAAAAAGTCTATCAAGTTAGTTAATTATCGAGACA
ACGCCCTCAGAAATGAGAGAACAGTACCCTCTTATCCTTGCTGCACTTTCCAGCACTGATACGCTGCCTAAAA
GAGGACTAGGGCACAGGTTTGAATTAATGTCACAAAACTGGATGGGCAAGTTACAACGGTGTTGATTAAGGAA
ACAGAACTCATGGTGCACCGGATATCTCCATCCTGATGAACCCTTGGAAAAATGCCAAAGATGCATATCCCCA
GGCAAATGCCTGATTAGTCTGGGATTGATAGATTGGTCTAGGATTCAGCCCTACTGGGAAGATGTCTAAATTA
TAATCAGTGTAGAAAGCGAAGTTCTCCTAGAAGAAGAGGCAAAGGTTAAAAAGAAGAAAAGAAAAGAAAGTGA
AGTCCTTTCTCCCCCAAAACCTCTCATCAATCAATCAGGGTAACAAACAGAACACTAGGGCTCTGTCTGTGGA
CCAAACCCAAAAGCCCTGCGGTCAGGGCCAGGAGGGTAGATCATGTGTTTGTGGCAACTTCCTCTGTGGGCTT
TTGCCCAGGTCTGTCCCCAAGCATACGATGGCCAAAACTTCTGCACCAGAGCAGCATCCTGTGTAACACAGTC
AGGTCCAGCAGTTAGGGAAAACTGCCCACTCAGAGTAGATAATATCTGGAAGGAATGACTGTTTGGGAAAAGT
TCCAATGCTAGTTCAGTGCCAACCCTTCCCCACCTTCTCCAGCTCTCTCCCACTGGTTCCTCCCCTCTCAACT
GCTCTGGTTCTTATAAAAACCTCACAGCCTTCCACTAACATCCCGTAGGAGCCTCTCTCCCTACTGCTGCTAC
ACAAGACCCTGAGACTGACCTGCAGGACGAAACCCTCTGGCTAACTAGAGAACCCACTGCTTACTGGCTTATC

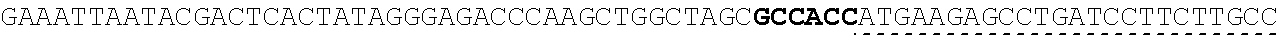

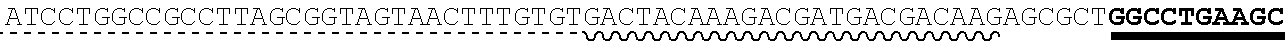

CAAGCTGCGCCAAGGAGGTGAAGAGCTGCAAGGGCAGATGTTTCGAACGGACCTTCGGCAACTGCAGATGCGA
CGCCGCCTGCGTGGAACTGGGAAACTGTTGTCTGGACTACCAGGAGACATGTATCGAGCCTGAGCACATCTGG
ACATGCAACAAGTTCAGATGTGGCGAGAAAAGACTGACTCGGAGTCTGTGCGCCTGTTCTGACGACTGCAAGG
ACAAGGGCGATTGCTGCATCAACTACAGCAGCGTGTGCCAGGGCGAAAAGAGCTGGGTCGAGGAACCCTGCGA
GAGCATCAACGAGCCCCAGTGCCCTGCCGGATTTGAGACACCTCCTACCCTGCTCTTCTCCCTGGATGGCTTC
AGAGCTGAGTACCTGCATACATGGGGAGGACTGCTGCCTGTGATCAGCAAGCTGAAGAAGTGCGGCACCTACA
CAAAGAACATGCGGCCTGTCTACCCTACCAAGACCTTCCCCAACCACTACAGCATCGTGACCGGCCTGTACCC
CGAGAGCCACGGCATCATCGACAACAAGATGTACGACCCCAAAATGAACGCCAGCTTCAGCCTGAAATCTAAG
GAAAAGTTCAACCCTGAGTGGTATAAGGGCGAGCCAATCTGGGTGACAGCCAAGTACCAGGGCCTGAAGAGCG
GCACCTTCTTCTGGCCTGGCAGCGACGTGGAGATCAACGGAATCTTCCCTGATATCTACAAGATGTATAACGG
CTCCGTGCCTTTCGAGGAGCGGATCTTGGCTGTTCTTCAGTGGCTGCAGCTGCCCAAGGACGAGAGACCCCAC
TTCTACACCCTGTACCTGGAAGAACCTGACAGCAGCGGCCACAGCTACGGCCCTGTGTCTAGCGAGGTGATCA
AGGCCCTGCAAAGAGTGGATGGCATGGTGGGCATGCTGATGGATGGACTGAAGGAACTGAACCTCCATCGTTG
CCTGAACCTGATCCTGATCTCCGACCACGGCATGGAACAGGGCAGCTGTAAAAAGTACATCTACCTGAACAAG
TACCTGGGCGACGTGAAGAATATCAAGGTGATCTACGGGCCGGCCGCCAGGCTGCGGCCTAGCGACGTGCCCG
ACAAGTATTACAGCTTCAACTACGAGGGCATCGCCAGAAACCTGTCTTGCAGAGAACCCAATCAACACTTCAA
GCCTTACCTGAAGCACTTTCTGCCTAAGAGACTGCACTTCGCCAAAAGCGACAGAATTGAACCCCTGACATTC
TATCTGGACCCTCAGTGGCAGCTGGCCCTCAATCCTTCTGAACGCAAATACTGCGGTAGCGGATTTCACGGCT
CTGATAACGTGTTTTCTAACATGCAGGCCCTGTTTGTGGGCTACGGCCCTGGCTTCAAGCACGGAATCGAGGC
TGACACCTTCGAGAACATCGAAGTTTACAATCTGATGTGCGACCTGCTGAATCTGACCCCAGCCCCTAACAAC
GGCACACACGGCTCTCTGAATCACCTGCTCAAGAACCCCGTGTATACCCCCAAACACCCTAAAGAGGTCCATC
CACTGGTGCAGTGCCCATTCACCAGAAACCCTCGGGACAACCTTGGCTGCAGCTGCAACCCTAGCATCCTCCC
TATCGAAGATTTCCAGACCCAGTTTAACCTGACTGTCGCCGAAGAGAAGATCATCAAGCACGAAACCCTGCCC
TACGGGAGGCCCAGAGTGCTGCAGAAAGAAAACACAATCTGTCTGCTGAGCCAGCACCAGTTTATGAGCGGCT
ACAGTCAGGACATCCTGATGCCTCTGTGGACCAGCTACACCGTGGACCGGAATGACAGCTTCAGCACCGAGGA
CTTCAGCAACTGCCTGTATCAGGACTTCCGCATCCCTCTGTCCCCTGTGCATAAGTGCAGCTTTTACAAGAAC
AACACCAAGGTGAGCTACGGGTTTCTGAGCCCACCACAACTGAACAAAAATAGCTCTGGCATCTACTCTGAGG
CGCTGCTGACCACCAACATCGTGCCAATGTACCAGAGCTTCCAGGTGATCTGGCGGTACTTCCATGATACCCT
GCTTCGGAAGTACGCCGAAGAACGGAACGGCGTGAACGTGGTGTCCGGCCCAGTGTTCGACTTCGACTACGAC
GGCAGATGTGATTCCCTTGAGAATCTGAGACAGAAGCGGCGGGTTATCAGAAACCAGGAGATCCTGATTCCTA
CCCACTTCTTCATCGTGCTGACATCTTGCAAGGATACCAGCCAAACACCTCTGCACTGCGAGAACCTGGACAC
CCTGGCCTTCATCCTGCCTCACCGGACCGATAATAGCGAATCCTGTGTGCACGGCAAACACGACTCTAGCTGG
GTGGAAGAGCTGCTGATGCTGCACAGAGCCCGGATCACAGATGTGGAACACATCACCGGCCTGTCCTTCTACC

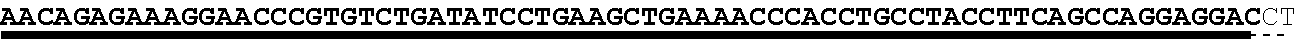

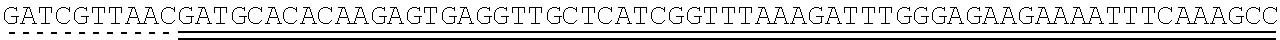

TTGGTGTTGATTGCCTTTGCTCAGTATCTTCAGCAGTGTCCATTTGAAGATCATGTAAAATTAGTGAATGAAG
TAACTGAATTTGCAAAAACATGTGTTGCTGATGAGTCAGCTGAAAATTGTGACAAATCACTTCATACCCTTTT
TGGAGACAAATTATGCACAGTTGCAACTCTTCGTGAAACCTATGGTGAAATGGCTGACTGCTGTGCAAAACAA
GAACCTGAGAGAAATGAATGCTTCTTGCAACACAAAGATGACAACCCAAACCTCCCCCGATTGGTGAGACCAG
AGGTTGATGTGATGTGCACTGCTTTTCATGACAATGAAGAGACATTTTTGAAAAAATACTTATATGAAATTGC
CAGAAGACATCCTTACTTTTATGCCCCGGAACTCCTTTTCTTTGCTAAAAGGTATAAAGCTGCTTTTACAGAA
TGTTGCCAAGCTGCTGATAAAGCTGCCTGCCTGTTGCCAAAGCTCGATGAACTTCGGGATGAAGGGAAGGCTT
CGTCTGCCAAACAGAGACTCAAGTGTGCCAGTCTCCAAAAATTTGGAGAAAGAGCTTTCAAAGCATGGGCAGT
AGCTCGCCTGAGCCAGAGATTTCCCAAAGCTGAGTTTGCAGAAGTTTCCAAGTTAGTGACAGATCTTACCAAA
GTCCACACGGAATGCTGCCATGGAGATCTGCTTGAATGTGCTGATGACAGGGCGGACCTTGCCAAGTATATCT
GTGAAAATCAAGATTCGATCTCCAGTAAACTGAAGGAATGCTGTGAAAAACCTCTGTTGGAAAAATCCCACTG
CATTGCCGAAGTGGAAAATGATGAGATGCCTGCTGACTTGCCTTCATTAGCTGCTGATTTTGTTGAAAGTAAG
GATGTTTGCAAAAACTATGCTGAGGCAAAGGATGTCTTCCTGGGCATGTTTTTGTATGAATATGCAAGAAGGC
ATCCTGATTACTCTGTCGTGCTGCTGCTGAGACTTGCCAAGACATATGAAACCACACTCGAGAAGTGCTGTGC
CGCTGCAGATCCTCATGAATGCTATGCCAAAGTGTTCGATGAATTTAAACCTCTTGTGGAAGAGCCTCAGAAT
TTAATCAAACAAAATTGTGAGCTTTTTGAGCAGCTTGGAGAGTACAAATTCCAGAATGCGCTATTAGTTCGTT
ACACCAAGAAAGTACCCCAAGTGTCAACTCCAACTCTTGTAGAGGTCTCAAGAAACCTAGGAAAAGTGGGCAG
CAAATGTTGTAAACATCCTGAAGCAAAAAGAATGCCCTGTGCAGAAGACTATCTATCCGTGGTCCTGAACCAG
TTATGTGTGTTGCATGAGAAAACGCCAGTAAGTGACAGAGTCACCAAATGCTGCACAGAATCCTTGGTGAACA
GGCGACCATGCTTTTCAGCTCTGGAAGTCGATGAAACATACGTTCCCAAAGAGTTTAATGCTGAAACATTCAC
CTTCCATGCAGATATATGCACACTTTCTGAGAAGGAGAGACAAATCAAGAAACAAACTGCACTTGTTGAGCTC
GTGAAACACAAGCCCAAGGCAACAAAAGAGCAACTGAAAGCTGTTATGGATGATTTCGCAGCTTTTGTAGAGA
AGTGCTGCAAGGCTGACGATAAGGAGACCTGCTTTGCCGAGGAGGGTAAAAAACTTGTTGCTGCAAGTCAAGC

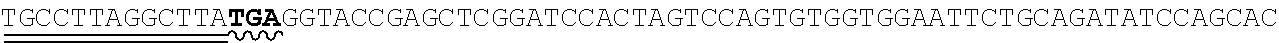

AGTGGCGGCCGCTCGAGTCTAGAGGGCCCGTTTAAACCCGCTGATCAGCCTCGACTGTGCCTTCTAGTTGCCA
GCCATCTGTTGTTTGCCCCTCCCCCGTGCCTTCCTTGACCCTGGAAGGTGCCACTCCCACTGTCCTTTCCTAA
TAAAATGAGGAAATTGCATCGCATTGTCTGAGTAGGTGTCATTCTATTCTGGGGGGTGGGGTGGGGCAGGACA
GCAAGGGGGAGGATTGGGAAGACAATAGCAGGCATGCTGGGGATGCGGTGGGCTCTATGGCTTCTGAGGCGGA
AAGAACCAGCTGGGGCTCTAGGGGGTATCCCCACGCGCCCTGTAGCGGCGCATTAAGCGCGGCGGGTGTGGTG
GTTACGCGCAGCGTGACCGCTACACTTGCCAGCGCCCTAGCGCCCGCTCCTTTCGCTTTCTTCCCTTCCTTTC
TCGCCACGTTCGCCGGCTTTCCCCGTCAAGCTCTAAATCGGGGGCTCCCTTTAGGGTTCCGATTTAGTGCTTT
ACGGCACCTCGACCCCAAAAAACTTGATTAGGGTGATGGTTCACGTAGTGGGCCATCGCCCTGATAGACGGTT
TTTCGCCCTTTGACGTTGGAGTCCACGTTCTTTAATAGTGGACTCTTGTTCCAAACTGGAACAACACTCAACC
CTATCTCGGTCTATTCTTTTGATTTATAAGGGATTTTGCCGATTTCGGCCTATTGGTTAAAAAATGAGCTGAT
TTAACAAAAATTTAACGCGAATTAATTCTGTGGAATGTGTGTCAGTTAGGGTGTGGAAAGTCCCCAGGCTCCC
CAGCAGGCAGAAGTATGCAAAGCATGCATCTCAATTAGTCAGCAACCAGGTGTGGAAAGTCCCCAGGCTCCCC
AGCAGGCAGAAGTATGCAAAGCATGCATCTCAATTAGTCAGCAACCATAGTCCCGCCCCTAACTCCGCCCATC
CCGCCCCTAACTCCGCCCAGTTCCGCCCATTCTCCGCCCCATGGCTGACTAATTTTTTTTATTTATGCAGAGG
CCGAGGCCGCCTCTGCCTCTGAGCTATTCCAGAAGTAGTGAGGAGGCTTTTTTGGAGGCCTAGGCTTTTGCAA
AAAGCTCCCGGGAGCTTGTATATCCATTTTCGGATCTGATCAAGAGACAGGATGAGGATCGTTTCGCATGATT
GAACAAGATGGATTGCACGCAGGTTCTCCGGCCGCTTGGGTGGAGAGGCTATTCGGCTATGACTGGGCACAAC
AGACAATCGGCTGCTCTGATGCCGCCGTGTTCCGGCTGTCAGCGCAGGGGCGCCCGGTTCTTTTTGTCAAGAC
CGACCTGTCCGGTGCCCTGAATGAACTGCAGGACGAGGCAGCGCGGCTATCGTGGCTGGCCACGACGGGCGTT
CCTTGCGCAGCTGTGCTCGACGTTGTCACTGAAGCGGGAAGGGACTGGCTGCTATTGGGCGAAGTGCCGGGGC
AGGATCTCCTGTCATCTCACCTTGCTCCTGCCGAGAAAGTATCCATCATGGCTGATGCAATGCGGCGGCTGCA
TACGCTTGATCCGGCTACCTGCCCATTCGACCACCAAGCGAAACATCGCATCGAGCGAGCACGTACTCGGATG
GAAGCCGGTCTTGTCGATCAGGATGATCTGGACGAAGAGCATCAGGGGCTCGCGCCAGCCGAACTGTTCGCCA
GGCTCAAGGCGCGCATGCCCGACGGCGAGGATCTCGTCGTGACCCATGGCGATGCCTGCTTGCCGAATATCAT
GGTGGAAAATGGCCGCTTTTCTGGATTCATCGACTGTGGCCGGCTGGGTGTGGCGGACCGCTATCAGGACATA
GCGTTGGCTACCCGTGATATTGCTGAAGAGCTTGGCGGCGAATGGGCTGACCGCTTCCTCGTGCTTTACGGTA
TCGCCGCTCCCGATTCGCAGCGCATCGCCTTCTATCGCCTTCTTGACGAGTTCTTCTGAGCGGGACTCTGGGG
TTCGAAATGACCGACCAAGCGACGCCCAACCTGCCATCACGAGATTTCGATTCCACCGCCGCCTTCTATGAAA
GGTTGGGCTTCGGAATCGTTTTCCGGGACGCCGGCTGGATGATCCTCCAGCGCGGGGATCTCATGCTGGAGTT
CTTCGCCCACCCCAACTTGTTTATTGCAGCTTATAATGGTTACAAATAAAGCAATAGCATCACAAATTTCACA
AATAAAGCATTTTTTTCACTGCATTCTAGTTGTGGTTTGTCCAAACTCATCAATGTATCTTATCATGTCTGTA
TACCGTCGACCTCTAGCTAGAGCTTGGCGTAATCATGGTCATAGCTGTTTCCTGTGTGAAATTGTTATCCGCT
CACAATTCCACACAACATACGAGCCGGAAGCATAAAGTGTAAAGCCTGGGGTGCCTAATGAGTGAGCTAACTC
ACATTAATTGCGTTGCGCTCACTGCCCGCTTTCCAGTCGGGAAACCTGTCGTGCCAGCTGCATTAATGAATCG
GCCAACGCGCGGGGAGAGGCGGTTTGCGTATTGGGCGCTCTTCCGCTTCCTCGCTCACTGACTCGCTGCGCTC
GGTCGTTCGGCTGCGGCGAGCGGTATCAGCTCACTCAAAGGCGGTAATACGGTTATCCACAGAATCAGGGGAT
AACGCAGGAAAGAACATGTGAGCAAAAGGCCAGCAAAAGGCCAGGAACCGTAAAAAGGCCGCGTTGCTGGCGT
TTTTCCATAGGCTCCGCCCCCCTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTCCTGTTCCGACCCTGCCGCTT
ACCGGATACCTGTCCGCCTTTCTCCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCCGTTCAGCCCGACCGCTGCGCCTT
ATCCGGTAACTATCGTCTTGAGTCCAACCCGGTAAGACACGACTTATCGCCACTGGCAGCAGCCACTGGTAAC
AGGATTAGCAGAGCGAGGTATGTAGGCGGTGCTACAGAGTTCTTGAAGTGGTGGCCTAACTACGGCTACACTA
GAAGAACAGTATTTGGTATCTGCGCTCTGCTGAAGCCAGTTACCTTCGGAAAAAGAGTTGGTAGCTCTTGATC
CGGCAAACAAACCACCGCTGGTAGCGGTGGTTTTTTTGTTTGCAAGCAGCAGATTACGCGCAGAAAAAAAGGA
TCTCAAGAAGATCCTTTGATCTTTTCTACGGGGTCTGACGCTCAGTGGAACGAAAACTCACGTTAAGGGATTT
TGGTCATGAGATTATCAAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGGGAGGGCTTACCATCTG
GCCCCAGTGCTGCAATGATACCGCGAGACCCACGCTCACCGGCTCCAGATTTATCAGCAATAAACCAGCCAGC
CGGAAGGGCCGAGCGCAGAAGTGGTCCTGCAACTTTATCCGCCTCCATCCAGTCTATTAATTGTTGCCGGGAA
GCTAGAGTAAGTAGTTCGCCAGTTAATAGTTTGCGCAACGTTGTTGCCATTGCTACAGGCATCGTGGTGTCAC
GCTCGTCGTTTGGTATGGCTTCATTCAGCTCCGGTTCCCAACGATCAAGGCGAGTTACATGATCCCCCATGTT
GTGCAAAAAAGCGGTTAGCTCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGCTTTTCTGTGACTGGTGAGT
ACTCAACCAAGTCATTCTGAGAATAGTGTATGCGGCGACCGAGTTGCTCTTGCCCGGCGTCAATACGGGATAA
TACCGCGCCACATAGCAGAACTTTAAAAGTGCTCATCATTGGAAAACGTTCTTCGGGGCGAAAACTCTCAAGG
ATCTTACCGCTGTTGAGATCCAGTTCGATGTAACCCACTCGTGCACCCAACTGATCTTCAGCATCTTTTACTT
TCACCAGCGTTTCTGGGTGAGCAAAAACAGGAAGGCAAAATGCCGCAAAAAAGGGAATAAGGGCGACACGGAA
ATGTTGAATACTCATACTCTTCCTTTTTCAATATTATTGAAGCATTTATCAGGGTTATTGTCTCATGAGCGGA
TACATATTTGAATGTATTTAGAAAAATAAACAAATAGGGGTTCCGCGCACATTTCCCCGAAAAGTGCCACCTG
ACGTC
CAG_Kozak_hAlb sig_FLAG_ENPPlopt_LINK_hAlb_stop_mir155_pcDNA3.1(+)

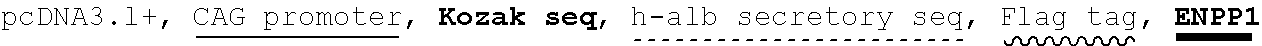

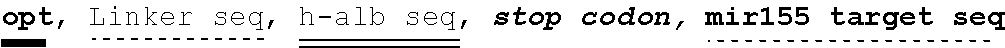

(SEQ ID NO: 98)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTGCGTTACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCATTGACGTCAATGGGTGGAG
TATTTACGGTAAACTGCCCACTTGGCAGTACATCAAGTGTATCATATGCCAAGTACGCCCCCTATTGACGTCA
ATGACGGTAAATGGCCCGCCTGGCATTATGCCCAGTACATGACCTTATGGGACTTTCCTACTTGGCAGTACAT
CTACGTATTAGTCATCGCTATTACCATGGTCGAGGTGAGCCCCACGTTCTGCTTCACTCTCCCCATCTCCCCC
CCCTCCCCACCCCCAATTTTGTATTTATTTATTTTTTAATTATTTTGTGCAGCGATGGGGGGGGGGGGGGGGG
GGGGGCGCGCGCCAGGCGGGGCGGGGCGGGGCGAGGGGCGGGGCGGGGCGAGGCGGAGAGGTGCGGCGGCAGC
CAATCAGAGCGGCGCGCTCCGAAAGTTTCCTTTTATGGCGAGGCGGCGGCGGCGGCGGCCCTATAAAAAGCGA
AGCGCGCGGCGGGCGCTCTGGCTAACTAGAGAACCCACTGCTTACTGGCTTATCGAAATTAATACGACTCACT

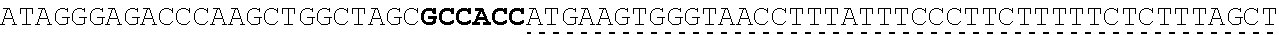

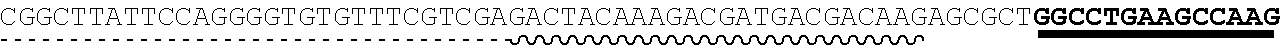

CTGCGCCAAGGAGGTGAAGAGCTGCAAGGGCAGATGTTTCGAACGGACCTTCGGCAACTGCAGATGCGACGCC
GCCTGCGTGGAACTGGGAAACTGTTGTCTGGACTACCAGGAGACATGTATCGAGCCTGAGCACATCTGGACAT
GCAACAAGTTCAGATGTGGCGAGAAAAGACTGACTCGGAGTCTGTGCGCCTGTTCTGACGACTGCAAGGACAA
GGGCGATTGCTGCATCAACTACAGCAGCGTGTGCCAGGGCGAAAAGAGCTGGGTCGAGGAACCCTGCGAGAGC
ATCAACGAGCCCCAGTGCCCTGCCGGATTTGAGACACCTCCTACCCTGCTCTTCTCCCTGGATGGCTTCAGAG
CTGAGTACCTGCATACATGGGGAGGACTGCTGCCTGTGATCAGCAAGCTGAAGAAGTGCGGCACCTACACAAA
GAACATGCGGCCTGTCTACCCTACCAAGACCTTCCCCAACCACTACAGCATCGTGACCGGCCTGTACCCCGAG
AGCCACGGCATCATCGACAACAAGATGTACGACCCCAAAATGAACGCCAGCTTCAGCCTGAAATCTAAGGAAA
AGTTCAACCCTGAGTGGTATAAGGGCGAGCCAATCTGGGTGACAGCCAAGTACCAGGGCCTGAAGAGCGGCAC
CTTCTTCTGGCCTGGCAGCGACGTGGAGATCAACGGAATCTTCCCTGATATCTACAAGATGTATAACGGCTCC
GTGCCTTTCGAGGAGCGGATCTTGGCTGTTCTTCAGTGGCTGCAGCTGCCCAAGGACGAGAGACCCCACTTCT
ACACCCTGTACCTGGAAGAACCTGACAGCAGCGGCCACAGCTACGGCCCTGTGTCTAGCGAGGTGATCAAGGC
CCTGCAAAGAGTGGATGGCATGGTGGGCATGCTGATGGATGGACTGAAGGAACTGAACCTCCATCGTTGCCTG
AACCTGATCCTGATCTCCGACCACGGCATGGAACAGGGCAGCTGTAAAAAGTACATCTACCTGAACAAGTACC
TGGGCGACGTGAAGAATATCAAGGTGATCTACGGGCCGGCCGCCAGGCTGCGGCCTAGCGACGTGCCCGACAA
GTATTACAGCTTCAACTACGAGGGCATCGCCAGAAACCTGTCTTGCAGAGAACCCAATCAACACTTCAAGCCT
TACCTGAAGCACTTTCTGCCTAAGAGACTGCACTTCGCCAAAAGCGACAGAATTGAACCCCTGACATTCTATC
TGGACCCTCAGTGGCAGCTGGCCCTCAATCCTTCTGAACGCAAATACTGCGGTAGCGGATTTCACGGCTCTGA
TAACGTGTTTTCTAACATGCAGGCCCTGTTTGTGGGCTACGGCCCTGGCTTCAAGCACGGAATCGAGGCTGAC
ACCTTCGAGAACATCGAAGTTTACAATCTGATGTGCGACCTGCTGAATCTGACCCCAGCCCCTAACAACGGCA
CACACGGCTCTCTGAATCACCTGCTCAAGAACCCCGTGTATACCCCCAAACACCCTAAAGAGGTCCATCCACT
GGTGCAGTGCCCATTCACCAGAAACCCTCGGGACAACCTTGGCTGCAGCTGCAACCCTAGCATCCTCCCTATC
GAAGATTTCCAGACCCAGTTTAACCTGACTGTCGCCGAAGAGAAGATCATCAAGCACGAAACCCTGCCCTACG
GGAGGCCCAGAGTGCTGCAGAAAGAAAACACAATCTGTCTGCTGAGCCAGCACCAGTTTATGAGCGGCTACAG
TCAGGACATCCTGATGCCTCTGTGGACCAGCTACACCGTGGACCGGAATGACAGCTTCAGCACCGAGGACTTC
AGCAACTGCCTGTATCAGGACTTCCGCATCCCTCTGTCCCCTGTGCATAAGTGCAGCTTTTACAAGAACAACA
CCAAGGTGAGCTACGGGTTTCTGAGCCCACCACAACTGAACAAAAATAGCTCTGGCATCTACTCTGAGGCGCT
GCTGACCACCAACATCGTGCCAATGTACCAGAGCTTCCAGGTGATCTGGCGGTACTTCCATGATACCCTGCTT
CGGAAGTACGCCGAAGAACGGAACGGCGTGAACGTGGTGTCCGGCCCAGTGTTCGACTTCGACTACGACGGCA
GATGTGATTCCCTTGAGAATCTGAGACAGAAGCGGCGGGTTATCAGAAACCAGGAGATCCTGATTCCTACCCA
CTTCTTCATCGTGCTGACATCTTGCAAGGATACCAGCCAAACACCTCTGCACTGCGAGAACCTGGACACCCTG
GCCTTCATCCTGCCTCACCGGACCGATAATAGCGAATCCTGTGTGCACGGCAAACACGACTCTAGCTGGGTGG
AAGAGCTGCTGATGCTGCACAGAGCCCGGATCACAGATGTGGAACACATCACCGGCCTGTCCTTCTACCAACA

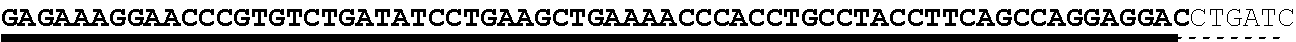

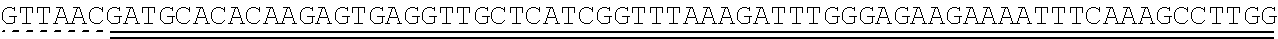

TGTTGATTGCCTTTGCTCAGTATCTTCAGCAGTGTCCATTTGAAGATCATGTAAAATTAGTGAATGAAGTAAC
TGAATTTGCAAAAACATGTGTTGCTGATGAGTCAGCTGAAAATTGTGACAAATCACTTCATACCCTTTTTGGA
GACAAATTATGCACAGTTGCAACTCTTCGTGAAACCTATGGTGAAATGGCTGACTGCTGTGCAAAACAAGAAC
CTGAGAGAAATGAATGCTTCTTGCAACACAAAGATGACAACCCAAACCTCCCCCGATTGGTGAGACCAGAGGT
TGATGTGATGTGCACTGCTTTTCATGACAATGAAGAGACATTTTTGAAAAAATACTTATATGAAATTGCCAGA
AGACATCCTTACTTTTATGCCCCGGAACTCCTTTTCTTTGCTAAAAGGTATAAAGCTGCTTTTACAGAATGTT
GCCAAGCTGCTGATAAAGCTGCCTGCCTGTTGCCAAAGCTCGATGAACTTCGGGATGAAGGGAAGGCTTCGTC
TGCCAAACAGAGACTCAAGTGTGCCAGTCTCCAAAAATTTGGAGAAAGAGCTTTCAAAGCATGGGCAGTAGCT
CGCCTGAGCCAGAGATTTCCCAAAGCTGAGTTTGCAGAAGTTTCCAAGTTAGTGACAGATCTTACCAAAGTCC
ACACGGAATGCTGCCATGGAGATCTGCTTGAATGTGCTGATGACAGGGCGGACCTTGCCAAGTATATCTGTGA
AAATCAAGATTCGATCTCCAGTAAACTGAAGGAATGCTGTGAAAAACCTCTGTTGGAAAAATCCCACTGCATT
GCCGAAGTGGAAAATGATGAGATGCCTGCTGACTTGCCTTCATTAGCTGCTGATTTTGTTGAAAGTAAGGATG
TTTGCAAAAACTATGCTGAGGCAAAGGATGTCTTCCTGGGCATGTTTTTGTATGAATATGCAAGAAGGCATCC
TGATTACTCTGTCGTGCTGCTGCTGAGACTTGCCAAGACATATGAAACCACACTCGAGAAGTGCTGTGCCGCT
GCAGATCCTCATGAATGCTATGCCAAAGTGTTCGATGAATTTAAACCTCTTGTGGAAGAGCCTCAGAATTTAA
TCAAACAAAATTGTGAGCTTTTTGAGCAGCTTGGAGAGTACAAATTCCAGAATGCGCTATTAGTTCGTTACAC
CAAGAAAGTACCCCAAGTGTCAACTCCAACTCTTGTAGAGGTCTCAAGAAACCTAGGAAAAGTGGGCAGCAAA
TGTTGTAAACATCCTGAAGCAAAAAGAATGCCCTGTGCAGAAGACTATCTATCCGTGGTCCTGAACCAGTTAT
GTGTGTTGCATGAGAAAACGCCAGTAAGTGACAGAGTCACCAAATGCTGCACAGAATCCTTGGTGAACAGGCG
ACCATGCTTTTCAGCTCTGGAAGTCGATGAAACATACGTTCCCAAAGAGTTTAATGCTGAAACATTCACCTTC
CATGCAGATATATGCACACTTTCTGAGAAGGAGAGACAAATCAAGAAACAAACTGCACTTGTTGAGCTCGTGA
AACACAAGCCCAAGGCAACAAAAGAGCAACTGAAAGCTGTTATGGATGATTTCGCAGCTTTTGTAGAGAAGTG
CTGCAAGGCTGACGATAAGGAGACCTGCTTTGCCGAGGAGGGTAAAAAACTTGTTGCTGCAAGTCAAGCTGCC

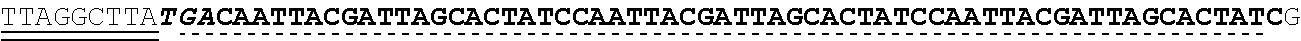

GTACCGAGCTCGGATCCACTAGTCCAGTGTGGTGGAATTCTGCAGATATCCAGCACAGTGGCGGCCGCTCGAG
TCTAGAGGGCCCGTTTAAACCCGCTGATCAGCCTCGACTGTGCCTTCTAGTTGCCAGCCATCTGTTGTTTGCC
CCTCCCCCGTGCCTTCCTTGACCCTGGAAGGTGCCACTCCCACTGTCCTTTCCTAATAAAATGAGGAAATTGC
ATCGCATTGTCTGAGTAGGTGTCATTCTATTCTGGGGGGTGGGGTGGGGCAGGACAGCAAGGGGGAGGATTGG
GAAGACAATAGCAGGCATGCTGGGGATGCGGTGGGCTCTATGGCTTCTGAGGCGGAAAGAACCAGCTGGGGCT
CTAGGGGGTATCCCCACGCGCCCTGTAGCGGCGCATTAAGCGCGGCGGGTGTGGTGGTTACGCGCAGCGTGAC
CGCTACACTTGCCAGCGCCCTAGCGCCCGCTCCTTTCGCTTTCTTCCCTTCCTTTCTCGCCACGTTCGCCGGC
TTTCCCCGTCAAGCTCTAAATCGGGGGCTCCCTTTAGGGTTCCGATTTAGTGCTTTACGGCACCTCGACCCCA
AAAAACTTGATTAGGGTGATGGTTCACGTAGTGGGCCATCGCCCTGATAGACGGTTTTTCGCCCTTTGACGTT
GGAGTCCACGTTCTTTAATAGTGGACTCTTGTTCCAAACTGGAACAACACTCAACCCTATCTCGGTCTATTCT
TTTGATTTATAAGGGATTTTGCCGATTTCGGCCTATTGGTTAAAAAATGAGCTGATTTAACAAAAATTTAACG
CGAATTAATTCTGTGGAATGTGTGTCAGTTAGGGTGTGGAAAGTCCCCAGGCTCCCCAGCAGGCAGAAGTATG
CAAAGCATGCATCTCAATTAGTCAGCAACCAGGTGTGGAAAGTCCCCAGGCTCCCCAGCAGGCAGAAGTATGC
AAAGCATGCATCTCAATTAGTCAGCAACCATAGTCCCGCCCCTAACTCCGCCCATCCCGCCCCTAACTCCGCC
CAGTTCCGCCCATTCTCCGCCCCATGGCTGACTAATTTTTTTTATTTATGCAGAGGCCGAGGCCGCCTCTGCC
TCTGAGCTATTCCAGAAGTAGTGAGGAGGCTTTTTTGGAGGCCTAGGCTTTTGCAAAAAGCTCCCGGGAGCTT
GTATATCCATTTTCGGATCTGATCAAGAGACAGGATGAGGATCGTTTCGCATGATTGAACAAGATGGATTGCA
CGCAGGTTCTCCGGCCGCTTGGGTGGAGAGGCTATTCGGCTATGACTGGGCACAACAGACAATCGGCTGCTCT
GATGCCGCCGTGTTCCGGCTGTCAGCGCAGGGGCGCCCGGTTCTTTTTGTCAAGACCGACCTGTCCGGTGCCC
TGAATGAACTGCAGGACGAGGCAGCGCGGCTATCGTGGCTGGCCACGACGGGCGTTCCTTGCGCAGCTGTGCT
CGACGTTGTCACTGAAGCGGGAAGGGACTGGCTGCTATTGGGCGAAGTGCCGGGGCAGGATCTCCTGTCATCT
CACCTTGCTCCTGCCGAGAAAGTATCCATCATGGCTGATGCAATGCGGCGGCTGCATACGCTTGATCCGGCTA
CCTGCCCATTCGACCACCAAGCGAAACATCGCATCGAGCGAGCACGTACTCGGATGGAAGCCGGTCTTGTCGA
TCAGGATGATCTGGACGAAGAGCATCAGGGGCTCGCGCCAGCCGAACTGTTCGCCAGGCTCAAGGCGCGCATG
CCCGACGGCGAGGATCTCGTCGTGACCCATGGCGATGCCTGCTTGCCGAATATCATGGTGGAAAATGGCCGCT
TTTCTGGATTCATCGACTGTGGCCGGCTGGGTGTGGCGGACCGCTATCAGGACATAGCGTTGGCTACCCGTGA
TATTGCTGAAGAGCTTGGCGGCGAATGGGCTGACCGCTTCCTCGTGCTTTACGGTATCGCCGCTCCCGATTCG
CAGCGCATCGCCTTCTATCGCCTTCTTGACGAGTTCTTCTGAGCGGGACTCTGGGGTTCGAAATGACCGACCA
AGCGACGCCCAACCTGCCATCACGAGATTTCGATTCCACCGCCGCCTTCTATGAAAGGTTGGGCTTCGGAATC
GTTTTCCGGGACGCCGGCTGGATGATCCTCCAGCGCGGGGATCTCATGCTGGAGTTCTTCGCCCACCCCAACT
TGTTTATTGCAGCTTATAATGGTTACAAATAAAGCAATAGCATCACAAATTTCACAAATAAAGCATTTTTTTC
ACTGCATTCTAGTTGTGGTTTGTCCAAACTCATCAATGTATCTTATCATGTCTGTATACCGTCGACCTCTAGC
TAGAGCTTGGCGTAATCATGGTCATAGCTGTTTCCTGTGTGAAATTGTTATCCGCTCACAATTCCACACAACA
TACGAGCCGGAAGCATAAAGTGTAAAGCCTGGGGTGCCTAATGAGTGAGCTAACTCACATTAATTGCGTTGCG
CTCACTGCCCGCTTTCCAGTCGGGAAACCTGTCGTGCCAGCTGCATTAATGAATCGGCCAACGCGCGGGGAGA
GGCGGTTTGCGTATTGGGCGCTCTTCCGCTTCCTCGCTCACTGACTCGCTGCGCTCGGTCGTTCGGCTGCGGC
GAGCGGTATCAGCTCACTCAAAGGCGGTAATACGGTTATCCACAGAATCAGGGGATAACGCAGGAAAGAACAT
GTGAGCAAAAGGCCAGCAAAAGGCCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAACCCGACAGGACTATAAAGATACC
AGGCGTTTCCCCCTGGAAGCTCCCTCGTGCGCTCTCCTGTTCCGACCCTGCCGCTTACCGGATACCTGTCCGC
CTTTCTCCCTTCGGGAAGCGTGGCGCTTTCTCATAGCTCACGCTGTAGGTATCTCAGTTCGGTGTAGGTCGTT
CGCTCCAAGCTGGGCTGTGTGCACGAACCCCCCGTTCAGCCCGACCGCTGCGCCTTATCCGGTAACTATCGTC
TTGAGTCCAACCCGGTAAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAGAAGAACAGTATTTGGT
ATCTGCGCTCTGCTGAAGCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGATCTCAAGAAGATCCTTT
GATCTTTTCTACGGGGTCTGACGCTCAGTGGAACGAAAACTCACGTTAAGGGATTTTGGTCATGAGATTATCA
AAAAGGATCTTCACCTAGATCCTTTTAAATTAAAAATGAAGTTTTAAATCAATCTAAAGTATATATGAGTAAA
CTTGGTCTGACAGTTACCAATGCTTAATCAGTGAGGCACCTATCTCAGCGATCTGTCTATTTCGTTCATCCAT
AGTTGCCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGAGCGCA
GAAGTGGTCCTGCAACTTTATCCGCCTCCATCCAGTCTATTAATTGTTGCCGGGAAGCTAGAGTAAGTAGTTC
GCCAGTTAATAGTTTGCGCAACGTTGTTGCCATTGCTACAGGCATCGTGGTGTCACGCTCGTCGTTTGGTATG
GCTTCATTCAGCTCCGGTTCCCAACGATCAAGGCGAGTTACATGATCCCCCATGTTGTGCAAAAAAGCGGTTA
GCTCCTTCGGTCCTCCGATCGTTGTCAGAAGTAAGTTGGCCGCAGTGTTATCACTCATGGTTATGGCAGCACT
GCATAATTCTCTTACTGTCATGCCATCCGTAAGATGCTTTTCTGTGACTGGTGAGTACTCAACCAAGTCATTC
TGAGAATAGTGTATGCGGCGACCGAGTTGCTCTTGCCCGGCGTCAATACGGGATAATACCGCGCCACATAGCA
GAACTTTAAAAGTGCTCATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTTCACCAGCGTTTCTGGG
TGAGCAAAAACAGGAAGGCAAAATGCCGCAAAAAAGGGAATAAGGGCGACACGGAAATGTTGAATACTCATAC
TCTTCCTTTTTCAATATTATTGAAGCATTTATCAGGGTTATTGTCTCATGAGCGGATACATATTTGAATGTAT
TTAGAAAAATAAACAAATAGGGGTTCCGCGCACATTTCCCCGAAAAGTGCCACCTGACGTC
CBA_Spacer_hAlb sig_FLAG_ENPPlopt_LINK_hAlb_SV40pA_pcDNA3.1(+)
(SEQ ID NO: 99)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTCTAGATGTACACTCGAGGTGAGCCCCACGTTCTGCTTCACTCTCCCCATCTCCCCCCC
CTCCCCACCCCCAATTTTGTATTTATTTATTTTTTAATTATTTTGTGCAGCGATGGGGGCGGGGGGGGGGGGG
GGGGCGCGCGCCAGGCGGGGCGGGGCGGGGCGAGGGGCGGGGCGGGGCGAGGCGGAGAGGTGCGGCGGCAGCC
AATCAGAGCGGCGCGCTCCGAAAGTTTCCTTTTATGGCGAGGCGGCGGCGGCGGCGGCCCTATAAAAAGCGAA
GCGCGCGGCGGGCGGCCACCCCGCGGGCCCGTTAATTAAACCGGTCGCCACCATGAAGTGGGTAACCTTTATT
TCCCTTCTTTTTCTCTTTAGCTCGGCTTATTCCAGGGGTGTGTTTCGTCGAGACTACAAAGACGATGACGACA
AGAGCGCTGGCCTGAAGCCAAGCTGCGCCAAGGAGGTGAAGAGCTGCAAGGGCAGATGTTTCGAACGGACCTT
CGGCAACTGCAGATGCGACGCCGCCTGCGTGGAACTGGGAAACTGTTGTCTGGACTACCAGGAGACATGTATC
GAGCCTGAGCACATCTGGACATGCAACAAGTTCAGATGTGGCGAGAAAAGACTGACTCGGAGTCTGTGCGCCT
GTTCTGACGACTGCAAGGACAAGGGCGATTGCTGCATCAACTACAGCAGCGTGTGCCAGGGCGAAAAGAGCTG
GGTCGAGGAACCCTGCGAGAGCATCAACGAGCCCCAGTGCCCTGCCGGATTTGAGACACCTCCTACCCTGCTC
TTCTCCCTGGATGGCTTCAGAGCTGAGTACCTGCATACATGGGGAGGACTGCTGCCTGTGATCAGCAAGCTGA
AGAAGTGCGGCACCTACACAAAGAACATGCGGCCTGTCTACCCTACCAAGACCTTCCCCAACCACTACAGCAT
CGTGACCGGCCTGTACCCCGAGAGCCACGGCATCATCGACAACAAGATGTACGACCCCAAAATGAACGCCAGC
TTCAGCCTGAAATCTAAGGAAAAGTTCAACCCTGAGTGGTATAAGGGCGAGCCAATCTGGGTGACAGCCAAGT
ACCAGGGCCTGAAGAGCGGCACCTTCTTCTGGCCTGGCAGCGACGTGGAGATCAACGGAATCTTCCCTGATAT
CTACAAGATGTATAACGGCTCCGTGCCTTTCGAGGAGCGGATCTTGGCTGTTCTTCAGTGGCTGCAGCTGCCC
AAGGACGAGAGACCCCACTTCTACACCCTGTACCTGGAAGAACCTGACAGCAGCGGCCACAGCTACGGCCCTG
TGTCTAGCGAGGTGATCAAGGCCCTGCAAAGAGTGGATGGCATGGTGGGCATGCTGATGGATGGACTGAAGGA
ACTGAACCTCCATCGTTGCCTGAACCTGATCCTGATCTCCGACCACGGCATGGAACAGGGCAGCTGTAAAAAG
TACATCTACCTGAACAAGTACCTGGGCGACGTGAAGAATATCAAGGTGATCTACGGGCCGGCCGCCAGGCTGC
GGCCTAGCGACGTGCCCGACAAGTATTACAGCTTCAACTACGAGGGCATCGCCAGAAACCTGTCTTGCAGAGA
ACCCAATCAACACTTCAAGCCTTACCTGAAGCACTTTCTGCCTAAGAGACTGCACTTCGCCAAAAGCGACAGA
ATTGAACCCCTGACATTCTATCTGGACCCTCAGTGGCAGCTGGCCCTCAATCCTTCTGAACGCAAATACTGCG
GTAGCGGATTTCACGGCTCTGATAACGTGTTTTCTAACATGCAGGCCCTGTTTGTGGGCTACGGCCCTGGCTT
CAAGCACGGAATCGAGGCTGACACCTTCGAGAACATCGAAGTTTACAATCTGATGTGCGACCTGCTGAATCTG
ACCCCAGCCCCTAACAACGGCACACACGGCTCTCTGAATCACCTGCTCAAGAACCCCGTGTATACCCCCAAAC
ACCCTAAAGAGGTCCATCCACTGGTGCAGTGCCCATTCACCAGAAACCCTCGGGACAACCTTGGCTGCAGCTG
CAACCCTAGCATCCTCCCTATCGAAGATTTCCAGACCCAGTTTAACCTGACTGTCGCCGAAGAGAAGATCATC
AAGCACGAAACCCTGCCCTACGGGAGGCCCAGAGTGCTGCAGAAAGAAAACACAATCTGTCTGCTGAGCCAGC
ACCAGTTTATGAGCGGCTACAGTCAGGACATCCTGATGCCTCTGTGGACCAGCTACACCGTGGACCGGAATGA
CAGCTTCAGCACCGAGGACTTCAGCAACTGCCTGTATCAGGACTTCCGCATCCCTCTGTCCCCTGTGCATAAG
TGCAGCTTTTACAAGAACAACACCAAGGTGAGCTACGGGTTTCTGAGCCCACCACAACTGAACAAAAATAGCT
CTGGCATCTACTCTGAGGCGCTGCTGACCACCAACATCGTGCCAATGTACCAGAGCTTCCAGGTGATCTGGCG
GTACTTCCATGATACCCTGCTTCGGAAGTACGCCGAAGAACGGAACGGCGTGAACGTGGTGTCCGGCCCAGTG
TTCGACTTCGACTACGACGGCAGATGTGATTCCCTTGAGAATCTGAGACAGAAGCGGCGGGTTATCAGAAACC
AGGAGATCCTGATTCCTACCCACTTCTTCATCGTGCTGACATCTTGCAAGGATACCAGCCAAACACCTCTGCA
CTGCGAGAACCTGGACACCCTGGCCTTCATCCTGCCTCACCGGACCGATAATAGCGAATCCTGTGTGCACGGC
AAACACGACTCTAGCTGGGTGGAAGAGCTGCTGATGCTGCACAGAGCCCGGATCACAGATGTGGAACACATCA
CCGGCCTGTCCTTCTACCAACAGAGAAAGGAACCCGTGTCTGATATCCTGAAGCTGAAAACCCACCTGCCTAC
CTTCAGCCAGGAGGACCTGATCGTTAACGATGCACACAAGAGTGAGGTTGCTCATCGGTTTAAAGATTTGGGA
GAAGAAAATTTCAAAGCCTTGGTGTTGATTGCCTTTGCTCAGTATCTTCAGCAGTGTCCATTTGAAGATCATG
TAAAATTAGTGAATGAAGTAACTGAATTTGCAAAAACATGTGTTGCTGATGAGTCAGCTGAAAATTGTGACAA
ATCACTTCATACCCTTTTTGGAGACAAATTATGCACAGTTGCAACTCTTCGTGAAACCTATGGTGAAATGGCT
GACTGCTGTGCAAAACAAGAACCTGAGAGAAATGAATGCTTCTTGCAACACAAAGATGACAACCCAAACCTCC
CCCGATTGGTGAGACCAGAGGTTGATGTGATGTGCACTGCTTTTCATGACAATGAAGAGACATTTTTGAAAAA
ATACTTATATGAAATTGCCAGAAGACATCCTTACTTTTATGCCCCGGAACTCCTTTTCTTTGCTAAAAGGTAT
AAAGCTGCTTTTACAGAATGTTGCCAAGCTGCTGATAAAGCTGCCTGCCTGTTGCCAAAGCTCGATGAACTTC
GGGATGAAGGGAAGGCTTCGTCTGCCAAACAGAGACTCAAGTGTGCCAGTCTCCAAAAATTTGGAGAAAGAGC
TTTCAAAGCATGGGCAGTAGCTCGCCTGAGCCAGAGATTTCCCAAAGCTGAGTTTGCAGAAGTTTCCAAGTTA
GTGACAGATCTTACCAAAGTCCACACGGAATGCTGCCATGGAGATCTGCTTGAATGTGCTGATGACAGGGCGG
ACCTTGCCAAGTATATCTGTGAAAATCAAGATTCGATCTCCAGTAAACTGAAGGAATGCTGTGAAAAACCTCT
GTTGGAAAAATCCCACTGCATTGCCGAAGTGGAAAATGATGAGATGCCTGCTGACTTGCCTTCATTAGCTGCT
GATTTTGTTGAAAGTAAGGATGTTTGCAAAAACTATGCTGAGGCAAAGGATGTCTTCCTGGGCATGTTTTTGT
ATGAATATGCAAGAAGGCATCCTGATTACTCTGTCGTGCTGCTGCTGAGACTTGCCAAGACATATGAAACCAC
ACTCGAGAAGTGCTGTGCCGCTGCAGATCCTCATGAATGCTATGCCAAAGTGTTCGATGAATTTAAACCTCTT
GTGGAAGAGCCTCAGAATTTAATCAAACAAAATTGTGAGCTTTTTGAGCAGCTTGGAGAGTACAAATTCCAGA
ATGCGCTATTAGTTCGTTACACCAAGAAAGTACCCCAAGTGTCAACTCCAACTCTTGTAGAGGTCTCAAGAAA
CCTAGGAAAAGTGGGCAGCAAATGTTGTAAACATCCTGAAGCAAAAAGAATGCCCTGTGCAGAAGACTATCTA
TCCGTGGTCCTGAACCAGTTATGTGTGTTGCATGAGAAAACGCCAGTAAGTGACAGAGTCACCAAATGCTGCA
CAGAATCCTTGGTGAACAGGCGACCATGCTTTTCAGCTCTGGAAGTCGATGAAACATACGTTCCCAAAGAGTT
TAATGCTGAAACATTCACCTTCCATGCAGATATATGCACACTTTCTGAGAAGGAGAGACAAATCAAGAAACAA
ACTGCACTTGTTGAGCTCGTGAAACACAAGCCCAAGGCAACAAAAGAGCAACTGAAAGCTGTTATGGATGATT
TCGCAGCTTTTGTAGAGAAGTGCTGCAAGGCTGACGATAAGGAGACCTGCTTTGCCGAGGAGGGTAAAAAACT
TGTTGCTGCAAGTCAAGCTGCCTTAGGCTTATAAGGCGCGCCCACGTGTAAAACTTGTTTATTGCAGCTTATA
ATGGTTACAAATAAAGCAATAGCATCACAAATTTCACAAATAAAGCATTTTTTTCACTGCATTCTAGTTGTGG
TTTGTCCAAACTCATCAATGTATCTTATCATGTCTGGATCATCGATGCGGCCGCTCGAGTCTAGAGGGCCCGT
TTAAACCCGCTGATCAGCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATTGCATCGCATTGTCTGA
GTAGGTGTCATTCTATTCTGGGGGGTGGGGTGGGGCAGGACAGCAAGGGGGAGGATTGGGAAGACAATAGCAG
GCATGCTGGGGATGCGGTGGGCTCTATGGCTTCTGAGGCGGAAAGAACCAGCTGGGGCTCTAGGGGGTATCCC
CACGCGCCCTGTAGCGGCGCATTAAGCGCGGCGGGTGTGGTGGTTACGCGCAGCGTGACCGCTACACTTGCCA
GCGCCCTAGCGCCCGCTCCTTTCGCTTTCTTCCCTTCCTTTCTCGCCACGTTCGCCGGCTTTCCCCGTCAAGC
TCTAAATCGGGGGCTCCCTTTAGGGTTCCGATTTAGTGCTTTACGGCACCTCGACCCCAAAAAACTTGATTAG
GGTGATGGTTCACGTAGTGGGCCATCGCCCTGATAGACGGTTTTTCGCCCTTTGACGTTGGAGTCCACGTTCT
TTAATAGTGGACTCTTGTTCCAAACTGGAACAACACTCAACCCTATCTCGGTCTATTCTTTTGATTTATAAGG
GATTTTGCCGATTTCGGCCTATTGGTTAAAAAATGAGCTGATTTAACAAAAATTTAACGCGAATTAATTCTGT
GGAATGTGTGTCAGTTAGGGTGTGGAAAGTCCCCAGGCTCCCCAGCAGGCAGAAGTATGCAAAGCATGCATCT
CAATTAGTCAGCAACCAGGTGTGGAAAGTCCCCAGGCTCCCCAGCAGGCAGAAGTATGCAAAGCATGCATCTC
AATTAGTCAGCAACCATAGTCCCGCCCCTAACTCCGCCCATCCCGCCCCTAACTCCGCCCAGTTCCGCCCATT
CTCCGCCCCATGGCTGACTAATTTTTTTTATTTATGCAGAGGCCGAGGCCGCCTCTGCCTCTGAGCTATTCCA
GAAGTAGTGAGGAGGCTTTTTTGGAGGCCTAGGCTTTTGCAAAAAGCTCCCGGGAGCTTGTATATCCATTTTC
GGATCTGATCAAGAGACAGGATGAGGATCGTTTCGCATGATTGAACAAGATGGATTGCACGCAGGTTCTCCGG
CCGCTTGGGTGGAGAGGCTATTCGGCTATGACTGGGCACAACAGACAATCGGCTGCTCTGATGCCGCCGTGTT
CCGGCTGTCAGCGCAGGGGCGCCCGGTTCTTTTTGTCAAGACCGACCTGTCCGGTGCCCTGAATGAACTGCAG
GACGAGGCAGCGCGGCTATCGTGGCTGGCCACGACGGGCGTTCCTTGCGCAGCTGTGCTCGACGTTGTCACTG
AAGCGGGAAGGGACTGGCTGCTATTGGGCGAAGTGCCGGGGCAGGATCTCCTGTCATCTCACCTTGCTCCTGC
CGAGAAAGTATCCATCATGGCTGATGCAATGCGGCGGCTGCATACGCTTGATCCGGCTACCTGCCCATTCGAC
CACCAAGCGAAACATCGCATCGAGCGAGCACGTACTCGGATGGAAGCCGGTCTTGTCGATCAGGATGATCTGG
ACGAAGAGCATCAGGGGCTCGCGCCAGCCGAACTGTTCGCCAGGCTCAAGGCGCGCATGCCCGACGGCGAGGA
TCTCGTCGTGACCCATGGCGATGCCTGCTTGCCGAATATCATGGTGGAAAATGGCCGCTTTTCTGGATTCATC
GACTGTGGCCGGCTGGGTGTGGCGGACCGCTATCAGGACATAGCGTTGGCTACCCGTGATATTGCTGAAGAGC
TTGGCGGCGAATGGGCTGACCGCTTCCTCGTGCTTTACGGTATCGCCGCTCCCGATTCGCAGCGCATCGCCTT
CTATCGCCTTCTTGACGAGTTCTTCTGAGCGGGACTCTGGGGTTCGAAATGACCGACCAAGCGACGCCCAACC
TGCCATCACGAGATTTCGATTCCACCGCCGCCTTCTATGAAAGGTTGGGCTTCGGAATCGTTTTCCGGGACGC
CGGCTGGATGATCCTCCAGCGCGGGGATCTCATGCTGGAGTTCTTCGCCCACCCCAACTTGTTTATTGCAGCT
TATAATGGTTACAAATAAAGCAATAGCATCACAAATTTCACAAATAAAGCATTTTTTTCACTGCATTCTAGTT
GTGGTTTGTCCAAACTCATCAATGTATCTTATCATGTCTGTATACCGTCGACCTCTAGCTAGAGCTTGGCGTA
ATCATGGTCATAGCTGTTTCCTGTGTGAAATTGTTATCCGCTCACAATTCCACACAACATACGAGCCGGAAGC
ATAAAGTGTAAAGCCTGGGGTGCCTAATGAGTGAGCTAACTCACATTAATTGCGTTGCGCTCACTGCCCGCTT
TCCAGTCGGGAAACCTGTCGTGCCAGCTGCATTAATGAATCGGCCAACGCGCGGGGAGAGGCGGTTTGCGTAT
TGGGCGCTCTTCCGCTTCCTCGCTCACTGACTCGCTGCGCTCGGTCGTTCGGCTGCGGCGAGCGGTATCAGCT
CACTCAAAGGCGGTAATACGGTTATCCACAGAATCAGGGGATAACGCAGGAAAGAACATGTGAGCAAAAGGCC
AGCAAAAGGCCAGGAACCGTAAAAAGGCCGCGTTGCTGGCGTTTTTCCATAGGCTCCGCCCCCCTGACGAGCA
TCACAAAAATCGACGCTCAAGTCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTGTAGGTCGTTCGCTCCAAGCTGGG
CTGTGTGCACGAACCCCCCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAGCGAGGTATGTAGGCGGTG
CTACAGAGTTCTTGAAGTGGTGGCCTAACTACGGCTACACTAGAAGAACAGTATTTGGTATCTGCGCTCTGCT
GAAGCCAGTTACCTTCGGAAAAAGAGTTGGTAGCTCTTGATCCGGCAAACAAACCACCGCTGGTAGCGGTGGT
TTTTTTGTTTGCAAGCAGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTATCAAAAAGGATCTTCAC
CTAGATCCTTTTAAATTAAAAATGAAGTTTTAAATCAATCTAAAGTATATATGAGTAAACTTGGTCTGACAGT
TACCAATGCTTAATCAGTGAGGCACCTATCTCAGCGATCTGTCTATTTCGTTCATCCATAGTTGCCTGACTCC
CCGTCGTGTAGATAACTACGATACGGGAGGGCTTACCATCTGGCCCCAGTGCTGCAATGATACCGCGAGACCC
ACGCTCACCGGCTCCAGATTTATCAGCAATAAACCAGCCAGCCGGAAGGGCCGAGCGCAGAAGTGGTCCTGCA
ACTTTATCCGCCTCCATCCAGTCTATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCGTTTGGTATGGCTTCATTCAGCTC
CGGTTCCCAACGATCAAGGCGAGTTACATGATCCCCCATGTTGTGCAAAAAAGCGGTTAGCTCCTTCGGTCCT
CCGATCGTTGTCAGAAGTAAGTTGGCCGCAGTGTTATCACTCATGGTTATGGCAGCACTGCATAATTCTCTTA
CTGTCATGCCATCCGTAAGATGCTTTTCTGTGACTGGTGAGTACTCAACCAAGTCATTCTGAGAATAGTGTAT
GCGGCGACCGAGTTGCTCTTGCCCGGCGTCAATACGGGATAATACCGCGCCACATAGCAGAACTTTAAAAGTG
CTCATCATTGGAAAACGTTCTTCGGGGCGAAAACTCTCAAGGATCTTACCGCTGTTGAGATCCAGTTCGATGT
AACCCACTCGTGCACCCAACTGATCTTCAGCATCTTTTACTTTCACCAGCGTTTCTGGGTGAGCAAAAACAGG
AAGGCAAAATGCCGCAAAAAAGGGAATAAGGGCGACACGGAAATGTTGAATACTCATACTCTTCCTTTTTCAA
TATTATTGAAGCATTTATCAGGGTTATTGTCTCATGAGCGGATACATATTTGAATGTATTTAGAAAAATAAAC
AAATAGGGGTTCCGCGCACATTTCCCCGAAAAGTGCCACCTGACGTC
MimiH9Prom_kozak_hAlb sig_FLAG_ENPPlopt_LINK_hAlb_pcDNA3.1(+)
(SEQ ID NO: 100)
GACGGATCGGGAGATCTCCCGATCCCCTATGGTGCACTCTCAGTACAATCTGCTCTGATGCCGCATAGTTAAG
CCAGTATCTGCTCCCTGCTTGTGTGTTGGAGGTCGCTGAGTAGTGCGCGAGCAAAATTTAAGCTACAACAAGG
CAAGGCTTGACCGACAATTGCATGAAGAATCTGCTTAGGGTTAGGCGTTTTGCGCTGCTTCGCGATGTACGGG
CCAGATATACGCGTTGAAAACTACAATTGACTATCTCTAAATTTTAAAATGTGATGATAGTTACTACTTCAGT
GGTCCACACTGTTTCTATCCTTTGGAACATAAGATTTTGTTGTGTAGCTTAAGGAAGAGGAATTCAGCAGTTG
ACTATCTCGATTGTTCTGGGAATTTTGGAGGCTGAGGGGAAATTTTCACATATAAGCGCTGGGTTTGCTGGGG
TATTGAGAGTGACCTCTGGCTAACTAGAGAACCCACTGCTTACTGGCTTATCGAAATTAATACGACTCACTAT
AGGGAGACCCAAGCTGGCTAGCGCCACCATGAAGTGGGTAACCTTTATTTCCCTTCTTTTTCTCTTTAGCTCG
GCTTATTCCAGGGGTGTGTTTCGTCGAGACTACAAAGACGATGACGACAAGAGCGCTGGCCTGAAGCCAAGCT
GCGCCAAGGAGGTGAAGAGCTGCAAGGGCAGATGTTTCGAACGGACCTTCGGCAACTGCAGATGCGACGCCGC
CTGCGTGGAACTGGGAAACTGTTGTCTGGACTACCAGGAGACATGTATCGAGCCTGAGCACATCTGGACATGC
AACAAGTTCAGATGTGGCGAGAAAAGACTGACTCGGAGTCTGTGCGCCTGTTCTGACGACTGCAAGGACAAGG
GCGATTGCTGCATCAACTACAGCAGCGTGTGCCAGGGCGAAAAGAGCTGGGTCGAGGAACCCTGCGAGAGCAT
CAACGAGCCCCAGTGCCCTGCCGGATTTGAGACACCTCCTACCCTGCTCTTCTCCCTGGATGGCTTCAGAGCT
GAGTACCTGCATACATGGGGAGGACTGCTGCCTGTGATCAGCAAGCTGAAGAAGTGCGGCACCTACACAAAGA
ACATGCGGCCTGTCTACCCTACCAAGACCTTCCCCAACCACTACAGCATCGTGACCGGCCTGTACCCCGAGAG
CCACGGCATCATCGACAACAAGATGTACGACCCCAAAATGAACGCCAGCTTCAGCCTGAAATCTAAGGAAAAG
TTCAACCCTGAGTGGTATAAGGGCGAGCCAATCTGGGTGACAGCCAAGTACCAGGGCCTGAAGAGCGGCACCT
TCTTCTGGCCTGGCAGCGACGTGGAGATCAACGGAATCTTCCCTGATATCTACAAGATGTATAACGGCTCCGT
GCCTTTCGAGGAGCGGATCTTGGCTGTTCTTCAGTGGCTGCAGCTGCCCAAGGACGAGAGACCCCACTTCTAC
ACCCTGTACCTGGAAGAACCTGACAGCAGCGGCCACAGCTACGGCCCTGTGTCTAGCGAGGTGATCAAGGCCC
TGCAAAGAGTGGATGGCATGGTGGGCATGCTGATGGATGGACTGAAGGAACTGAACCTCCATCGTTGCCTGAA
CCTGATCCTGATCTCCGACCACGGCATGGAACAGGGCAGCTGTAAAAAGTACATCTACCTGAACAAGTACCTG
GGCGACGTGAAGAATATCAAGGTGATCTACGGGCCGGCCGCCAGGCTGCGGCCTAGCGACGTGCCCGACAAGT
ATTACAGCTTCAACTACGAGGGCATCGCCAGAAACCTGTCTTGCAGAGAACCCAATCAACACTTCAAGCCTTA
CCTGAAGCACTTTCTGCCTAAGAGACTGCACTTCGCCAAAAGCGACAGAATTGAACCCCTGACATTCTATCTG
GACCCTCAGTGGCAGCTGGCCCTCAATCCTTCTGAACGCAAATACTGCGGTAGCGGATTTCACGGCTCTGATA
ACGTGTTTTCTAACATGCAGGCCCTGTTTGTGGGCTACGGCCCTGGCTTCAAGCACGGAATCGAGGCTGACAC
CTTCGAGAACATCGAAGTTTACAATCTGATGTGCGACCTGCTGAATCTGACCCCAGCCCCTAACAACGGCACA
CACGGCTCTCTGAATCACCTGCTCAAGAACCCCGTGTATACCCCCAAACACCCTAAAGAGGTCCATCCACTGG
TGCAGTGCCCATTCACCAGAAACCCTCGGGACAACCTTGGCTGCAGCTGCAACCCTAGCATCCTCCCTATCGA
AGATTTCCAGACCCAGTTTAACCTGACTGTCGCCGAAGAGAAGATCATCAAGCACGAAACCCTGCCCTACGGG
AGGCCCAGAGTGCTGCAGAAAGAAAACACAATCTGTCTGCTGAGCCAGCACCAGTTTATGAGCGGCTACAGTC
AGGACATCCTGATGCCTCTGTGGACCAGCTACACCGTGGACCGGAATGACAGCTTCAGCACCGAGGACTTCAG
CAACTGCCTGTATCAGGACTTCCGCATCCCTCTGTCCCCTGTGCATAAGTGCAGCTTTTACAAGAACAACACC
AAGGTGAGCTACGGGTTTCTGAGCCCACCACAACTGAACAAAAATAGCTCTGGCATCTACTCTGAGGCGCTGC
TGACCACCAACATCGTGCCAATGTACCAGAGCTTCCAGGTGATCTGGCGGTACTTCCATGATACCCTGCTTCG
GAAGTACGCCGAAGAACGGAACGGCGTGAACGTGGTGTCCGGCCCAGTGTTCGACTTCGACTACGACGGCAGA
TGTGATTCCCTTGAGAATCTGAGACAGAAGCGGCGGGTTATCAGAAACCAGGAGATCCTGATTCCTACCCACT
TCTTCATCGTGCTGACATCTTGCAAGGATACCAGCCAAACACCTCTGCACTGCGAGAACCTGGACACCCTGGC
CTTCATCCTGCCTCACCGGACCGATAATAGCGAATCCTGTGTGCACGGCAAACACGACTCTAGCTGGGTGGAA
GAGCTGCTGATGCTGCACAGAGCCCGGATCACAGATGTGGAACACATCACCGGCCTGTCCTTCTACCAACAGA
GAAAGGAACCCGTGTCTGATATCCTGAAGCTGAAAACCCACCTGCCTACCTTCAGCCAGGAGGACCTGATCGT
TAACGATGCACACAAGAGTGAGGTTGCTCATCGGTTTAAAGATTTGGGAGAAGAAAATTTCAAAGCCTTGGTG
TTGATTGCCTTTGCTCAGTATCTTCAGCAGTGTCCATTTGAAGATCATGTAAAATTAGTGAATGAAGTAACTG
AATTTGCAAAAACATGTGTTGCTGATGAGTCAGCTGAAAATTGTGACAAATCACTTCATACCCTTTTTGGAGA
CAAATTATGCACAGTTGCAACTCTTCGTGAAACCTATGGTGAAATGGCTGACTGCTGTGCAAAACAAGAACCT
GAGAGAAATGAATGCTTCTTGCAACACAAAGATGACAACCCAAACCTCCCCCGATTGGTGAGACCAGAGGTTG
ATGTGATGTGCACTGCTTTTCATGACAATGAAGAGACATTTTTGAAAAAATACTTATATGAAATTGCCAGAAG
ACATCCTTACTTTTATGCCCCGGAACTCCTTTTCTTTGCTAAAAGGTATAAAGCTGCTTTTACAGAATGTTGC
CAAGCTGCTGATAAAGCTGCCTGCCTGTTGCCAAAGCTCGATGAACTTCGGGATGAAGGGAAGGCTTCGTCTG
CCAAACAGAGACTCAAGTGTGCCAGTCTCCAAAAATTTGGAGAAAGAGCTTTCAAAGCATGGGCAGTAGCTCG
CCTGAGCCAGAGATTTCCCAAAGCTGAGTTTGCAGAAGTTTCCAAGTTAGTGACAGATCTTACCAAAGTCCAC
ACGGAATGCTGCCATGGAGATCTGCTTGAATGTGCTGATGACAGGGCGGACCTTGCCAAGTATATCTGTGAAA
ATCAAGATTCGATCTCCAGTAAACTGAAGGAATGCTGTGAAAAACCTCTGTTGGAAAAATCCCACTGCATTGC
CGAAGTGGAAAATGATGAGATGCCTGCTGACTTGCCTTCATTAGCTGCTGATTTTGTTGAAAGTAAGGATGTT
TGCAAAAACTATGCTGAGGCAAAGGATGTCTTCCTGGGCATGTTTTTGTATGAATATGCAAGAAGGCATCCTG
ATTACTCTGTCGTGCTGCTGCTGAGACTTGCCAAGACATATGAAACCACACTCGAGAAGTGCTGTGCCGCTGC
AGATCCTCATGAATGCTATGCCAAAGTGTTCGATGAATTTAAACCTCTTGTGGAAGAGCCTCAGAATTTAATC
AAACAAAATTGTGAGCTTTTTGAGCAGCTTGGAGAGTACAAATTCCAGAATGCGCTATTAGTTCGTTACACCA
AGAAAGTACCCCAAGTGTCAACTCCAACTCTTGTAGAGGTCTCAAGAAACCTAGGAAAAGTGGGCAGCAAATG
TTGTAAACATCCTGAAGCAAAAAGAATGCCCTGTGCAGAAGACTATCTATCCGTGGTCCTGAACCAGTTATGT
GTGTTGCATGAGAAAACGCCAGTAAGTGACAGAGTCACCAAATGCTGCACAGAATCCTTGGTGAACAGGCGAC
CATGCTTTTCAGCTCTGGAAGTCGATGAAACATACGTTCCCAAAGAGTTTAATGCTGAAACATTCACCTTCCA
TGCAGATATATGCACACTTTCTGAGAAGGAGAGACAAATCAAGAAACAAACTGCACTTGTTGAGCTCGTGAAA
CACAAGCCCAAGGCAACAAAAGAGCAACTGAAAGCTGTTATGGATGATTTCGCAGCTTTTGTAGAGAAGTGCT
GCAAGGCTGACGATAAGGAGACCTGCTTTGCCGAGGAGGGTAAAAAACTTGTTGCTGCAAGTCAAGCTGCCTT
AGGCTTATGAGGTACCGAGCTCGGATCCACTAGTCCAGTGTGGTGGAATTCTGCAGATATCCAGCACAGTGGC
GGCCGCTCGAGTCTAGAGGGCCCGTTTAAACCCGCTGATCAGCCTCGACTGTGCCTTCTAGTTGCCAGCCATC
TGTTGTTTGCCCCTCCCCCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGGTGGGGCAGGACAGCAAGG
GGGAGGATTGGGAAGACAATAGCAGGCATGCTGGGGATGCGGTGGGCTCTATGGCTTCTGAGGCGGAAAGAAC
CAGCTGGGGCTCTAGGGGGTATCCCCACGCGCCCTGTAGCGGCGCATTAAGCGCGGCGGGTGTGGTGGTTACG
CGCAGCGTGACCGCTACACTTGCCAGCGCCCTAGCGCCCGCTCCTTTCGCTTTCTTCCCTTCCTTTCTCGCCA
CGTTCGCCGGCTTTCCCCGTCAAGCTCTAAATCGGGGGCTCCCTTTAGGGTTCCGATTTAGTGCTTTACGGCA
CCTCGACCCCAAAAAACTTGATTAGGGTGATGGTTCACGTAGTGGGCCATCGCCCTGATAGACGGTTTTTCGC
CCTTTGACGTTGGAGTCCACGTTCTTTAATAGTGGACTCTTGTTCCAAACTGGAACAACACTCAACCCTATCT
CGGTCTATTCTTTTGATTTATAAGGGATTTTGCCGATTTCGGCCTATTGGTTAAAAAATGAGCTGATTTAACA
AAAATTTAACGCGAATTAATTCTGTGGAATGTGTGTCAGTTAGGGTGTGGAAAGTCCCCAGGCTCCCCAGCAG
GCAGAAGTATGCAAAGCATGCATCTCAATTAGTCAGCAACCAGGTGTGGAAAGTCCCCAGGCTCCCCAGCAGG
CAGAAGTATGCAAAGCATGCATCTCAATTAGTCAGCAACCATAGTCCCGCCCCTAACTCCGCCCATCCCGCCC
CTAACTCCGCCCAGTTCCGCCCATTCTCCGCCCCATGGCTGACTAATTTTTTTTATTTATGCAGAGGCCGAGG
CCGCCTCTGCCTCTGAGCTATTCCAGAAGTAGTGAGGAGGCTTTTTTGGAGGCCTAGGCTTTTGCAAAAAGCT
CCCGGGAGCTTGTATATCCATTTTCGGATCTGATCAAGAGACAGGATGAGGATCGTTTCGCATGATTGAACAA
GATGGATTGCACGCAGGTTCTCCGGCCGCTTGGGTGGAGAGGCTATTCGGCTATGACTGGGCACAACAGACAA
TCGGCTGCTCTGATGCCGCCGTGTTCCGGCTGTCAGCGCAGGGGCGCCCGGTTCTTTTTGTCAAGACCGACCT
GTCCGGTGCCCTGAATGAACTGCAGGACGAGGCAGCGCGGCTATCGTGGCTGGCCACGACGGGCGTTCCTTGC
GCAGCTGTGCTCGACGTTGTCACTGAAGCGGGAAGGGACTGGCTGCTATTGGGCGAAGTGCCGGGGCAGGATC
TCCTGTCATCTCACCTTGCTCCTGCCGAGAAAGTATCCATCATGGCTGATGCAATGCGGCGGCTGCATACGCT
TGATCCGGCTACCTGCCCATTCGACCACCAAGCGAAACATCGCATCGAGCGAGCACGTACTCGGATGGAAGCC
GGTCTTGTCGATCAGGATGATCTGGACGAAGAGCATCAGGGGCTCGCGCCAGCCGAACTGTTCGCCAGGCTCA
AGGCGCGCATGCCCGACGGCGAGGATCTCGTCGTGACCCATGGCGATGCCTGCTTGCCGAATATCATGGTGGA
AAATGGCCGCTTTTCTGGATTCATCGACTGTGGCCGGCTGGGTGTGGCGGACCGCTATCAGGACATAGCGTTG
GCTACCCGTGATATTGCTGAAGAGCTTGGCGGCGAATGGGCTGACCGCTTCCTCGTGCTTTACGGTATCGCCG
CTCCCGATTCGCAGCGCATCGCCTTCTATCGCCTTCTTGACGAGTTCTTCTGAGCGGGACTCTGGGGTTCGAA
ATGACCGACCAAGCGACGCCCAACCTGCCATCACGAGATTTCGATTCCACCGCCGCCTTCTATGAAAGGTTGG
GCTTCGGAATCGTTTTCCGGGACGCCGGCTGGATGATCCTCCAGCGCGGGGATCTCATGCTGGAGTTCTTCGC
CCACCCCAACTTGTTTATTGCAGCTTATAATGGTTACAAATAAAGCAATAGCATCACAAATTTCACAAATAAA
GCATTTTTTTCACTGCATTCTAGTTGTGGTTTGTCCAAACTCATCAATGTATCTTATCATGTCTGTATACCGT
CGACCTCTAGCTAGAGCTTGGCGTAATCATGGTCATAGCTGTTTCCTGTGTGAAATTGTTATCCGCTCACAAT
TCCACACAACATACGAGCCGGAAGCATAAAGTGTAAAGCCTGGGGTGCCTAATGAGTGAGCTAACTCACATTA
ATTGCGTTGCGCTCACTGCCCGCTTTCCAGTCGGGAAACCTGTCGTGCCAGCTGCATTAATGAATCGGCCAAC
GCGCGGGGAGAGGCGGTTTGCGTATTGGGCGCTCTTCCGCTTCCTCGCTCACTGACTCGCTGCGCTCGGTCGT
TCGGCTGCGGCGAGCGGTATCAGCTCACTCAAAGGCGGTAATACGGTTATCCACAGAATCAGGGGATAACGCA
GGAAAGAACATGTGAGCAAAAGGCCAGCAAAAGGCCAGGAACCGTAAAAAGGCCGCGTTGCTGGCGTTTTTCC
ATAGGCTCCGCCCCCCTGACGAGCATCACAAAAATCGACGCTCAAGTCAGAGGTGGCGAAACCCGACAGGACT
ATAAAGATACCAGGCGTTTCCCCCTGGAAGCTCCCTCGTGCGCTCTCCTGTTCCGACCCTGCCGCTTACCGGA
TACCTGTCCGCCTTTCTCCCTTCGGGAAGCGTGGCGCTTTCTCATAGCTCACGCTGTAGGTATCTCAGTTCGG
TGTAGGTCGTTCGCTCCAAGCTGGGCTGTGTGCACGAACCCCCCGTTCAGCCCGACCGCTGCGCCTTATCCGG
TAACTATCGTCTTGAGTCCAACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAACTACGGCTACACTAGAAGAA
CAGTATTTGGTATCTGCGCTCTGCTGAAGCCAGTTACCTTCGGAAAAAGAGTTGGTAGCTCTTGATCCGGCAA
ACAAACCACCGCTGGTAGCGGTGGTTTTTTTGTTTGCAAGCAGCAGATTACGCGCAGAAAAAAAGGATCTCAA
GAAGATCCTTTGATCTTTTCTACGGGGTCTGACGCTCAGTGGAACGAAAACTCACGTTAAGGGATTTTGGTCA
TGAGATTATCAAAAAGGATCTTCACCTAGATCCTTTTAAATTAAAAATGAAGTTTTAAATCAATCTAAAGTAT
ATATGAGTAAACTTGGTCTGACAGTTACCAATGCTTAATCAGTGAGGCACCTATCTCAGCGATCTGTCTATTT
CGTTCATCCATAGTTGCCTGACTCCCCGTCGTGTAGATAACTACGATACGGGAGGGCTTACCATCTGGCCCCA
GTGCTGCAATGATACCGCGAGACCCACGCTCACCGGCTCCAGATTTATCAGCAATAAACCAGCCAGCCGGAAG
GGCCGAGCGCAGAAGTGGTCCTGCAACTTTATCCGCCTCCATCCAGTCTATTAATTGTTGCCGGGAAGCTAGA
GTAAGTAGTTCGCCAGTTAATAGTTTGCGCAACGTTGTTGCCATTGCTACAGGCATCGTGGTGTCACGCTCGT
CGTTTGGTATGGCTTCATTCAGCTCCGGTTCCCAACGATCAAGGCGAGTTACATGATCCCCCATGTTGTGCAA
AAAAGCGGTTAGCTCCTTCGGTCCTCCGATCGTTGTCAGAAGTAAGTTGGCCGCAGTGTTATCACTCATGGTT
ATGGCAGCACTGCATAATTCTCTTACTGTCATGCCATCCGTAAGATGCTTTTCTGTGACTGGTGAGTACTCAA
CCAAGTCATTCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATAAGGGCGACACGGAAATGTTG
AATACTCATACTCTTCCTTTTTCAATATTATTGAAGCATTTATCAGGGTTATTGTCTCATGAGCGGATACATA
TTTGAATGTATTTAGAAAAATAAACAAATAGGGGTTCCGCGCACATTTCCCCGAAAAGTGCCACCTGACGTC
pAAV-ENPP1 FL-miR122 3x BS
(SEQ ID NO: 101)
CCAAGTCATTCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATAAGGGCGACACGGAAATGTTG
AATACTCATACTCTTCCTTTTTCAATATTATTGAAGCATTTATCAGGGTTATTGTCTCATGAGCGGATACATA
TTTGAATGTATTTAGAAAAATAAACAAATAGGGGTTCCGCGCACATTTCCCCGAAAAGTGCCACCTGACGTCT
AAGAAACCATTATTATCATGACATTAACCTATAAAAATAGGCGTATCACGAGGCCCTTTCGTCTCGCGCGTTT
CGGTGATGACGGTGAAAACCTCTGACACATGCAGCTCCCGGAGACGGTCACAGCTTGTCTGTAAGCGGATGCC
GGGAGCAGACAAGCCCGTCAGGGCGCGTCAGCGGGTGTTGGCGGGTGTCGGGGCTGGCTTAACTATGCGGCAT
CAGAGCAGATTGTACTGAGAGTGCACCATATGCGGTGTGAAATACCGCACAGATGCGTAAGGAGAAAATACCG
CATCAGGAAATTGTAAGCGTTAATATTTTGTTAAAATTCGCGTTAAATTTTTGTTAAATCAGCTCATTTTTTA
ACCAATAGGCCGAAATCGGCAAAATCCCTTATAAATCAAAAGAATAGACCGAGATAGGGTTGAGTGTTGTTCC
AGTTTGGAACAAGAGTCCACTATTAAAGAACGTGGACTCCAACGTCAAAGGGCGAAAAACCGTCTATCAGGGC
GATGGCCCACTACGTGAACCATCACCCTAATCAAGTTTTTTGGGGTCGAGGTGCCGTAAAGCACTAAATCGGA
ACCCTAAAGGGAGCCCCCGATTTAGAGCTTGACGGGGAAAGCCGGCGAACGTGGCGAGAAAGGAAGGGAAGAA
AGCGAAAGGAGCGGGCGCTAGGGCGCTGGCAAGTGTAGCGGTCACGCTGCGCGTAACCACCACACCCGCCGCG
CTTAATGCGCCGCTACAGGGCGCGTCCATTCGCCATTCAGGCTGCGCAACTGTTGGGAAGGGCGATCGGTGCG
GGCCTCTTCGCTATTACGCCAGGCTGCAGGGGGGGGGGGGGGGGGGCCACTCCCTCTCTGCGCGCTCGCTCGC
TCACTGAGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTCAGATCTGAATTCGGTACCTAGTTATTAATAG
TAATCAATTACGGGGTCATTAGTTCATAGCCCATATATGGAGTTCCGCGTTACATAACTTACGGTAAATGGCC
CGCCTGGCTGACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACATCAAGTGTAT
CATATGCCAAGTACGCCCCCTATTGACGTCAATGACGGTAAATGGCCCGCCTGGCATTATGCCCAGTACATGA
CCTTATGGGACTTTCCTACTTGGCAGTACATCTACGTATTAGTCATCGCTATTACCATGGTCGAGGTGAGCCC
CACGTTCTGCTTCACTCTCCCCATCTCCCCCCCCTCCCCACCCCCAATTTTGTATTTATTTATTTTTTAATTA
TTTTGTGCAGCGATGGGGGCGGGGGGGGGGGGGGGCGCGCGCCAGGCGGGGGGGGCGGGGCGAGGGGGGGG
CGGGGCGAGGCGGAGAGGTGCGGCGGCAGCCAATCAGAGCGGCGCGCTCCGAAAGTTTCCTTTTATGGCGAGG
CGGCGGCGGCGGCGGCCCTATAAAAAGCGAAGCGCGCGGCGGGCGGGAGTCGCTGCGCGCTGCCTTCGCCCCG
TGCCCCGCTCCGCCGCCGCCTCGCGCCGCCCGCCCCGGCTCTGACTGACCGCGTTACTCCCACAGGTGAGCGG
GCGGGACGGCCCTTCTCCTCCGGGCTGTAATTAGCGCTTGGTTTAATGACGGCTTGTTTCTTTTCTGTGGCTG
CGTGAAAGCCTTGAGGGGCTCCGGGAGCTAGAGCCTCTGCTAACCATGTTCATGCCTTCTTCTTTTTCCTACA
GCTCCTGGGCAACGTGCTGGTTATTGTGCTGTCTCATCATTTTGGCAAAGAATTCCTCGAAGATCCGAAGGGG
TTCAAGCTTAGCGCCACCATGGAGCGCGACGGCTGCGCGGGGGGCGGGAGCCGCGGCGGCGAGGGGGGCGCG
CTCCCCGGGAGGGCCCGGCGGGGAACGGCCGCGATCGGGGCCGCAGCCACGCTGCCGAGGCGCCCGGGGACCC
GCAGGCCGCGGCGTCCTTGCTGGCCCCTATGGACGTGGGGGAGGAGCCGCTGGAGAAGGCGGCGCGCGCCCGC
ACTGCCAAGGACCCCAACACCTATAAAGTACTCTCGCTGGTATTGTCAGTATGTGTGTTAACAACAATACTTG
GTTGTATATTTGGGTTGAAACCAAGCTGTGCCAAAGAAGTTAAAAGTTGCAAAGGTCGCTGTTTCGAGAGAAC
ATTTGGGAACTGTCGCTGTGATGCTGCCTGTGTTGAGCTTGGAAACTGCTGTTTAGATTACCAGGAGACGTGC
ATAGAACCAGAACATATATGGACTTGCAACAAATTCAGGTGTGGTGAGAAAAGGTTGACCAGAAGCCTCTGTG
CCTGTTCAGATGACTGCAAGGACAAGGGCGACTGCTGCATCAACTACAGTTCTGTGTGTCAAGGTGAGAAAAG
TTGGGTAGAAGAACCATGTGAGAGCATTAATGAGCCACAGTGCCCAGCAGGGTTTGAAACGCCTCCTACCCTC
TTATTTTCTTTGGATGGATTCAGGGCAGAATATTTACACACTTGGGGTGGACTTCTTCCTGTTATTAGCAAAC
TAAAAAAATGTGGAACATATACTAAAAACATGAGACCGGTATATCCAACAAAAACTTTCCCCAATCACTACAG
CATTGTCACCGGATTGTATCCAGAATCTCATGGCATAATCGACAATAAAATGTATGATCCCAAAATGAATGCT
TCCTTTTCACTTAAAAGTAAAGAGAAATTTAATCCTGAGTGGTACAAAGGAGAACCAATTTGGGTCACAGCTA
AGTATCAAGGCCTCAAGTCTGGCACATTTTTCTGGCCAGGATCAGATGTGGAAATTAACGGAATTTTCCCAGA
CATCTATAAAATGTATAATGGTTCAGTACCATTTGAAGAAAGGATTTTAGCTGTTCTTCAGTGGCTACAGCTT
CCTAAAGATGAAAGACCACACTTTTACACTCTGTATTTAGAAGAACCAGATTCTTCAGGTCATTCATATGGAC
CAGTCAGCAGTGAAGTCATCAAAGCCTTGCAGAGGGTTGATGGTATGGTTGGTATGCTGATGGATGGTCTGAA
AGAGCTGAACTTGCACAGATGCCTGAACCTCATCCTTATTTCAGATCATGGCATGGAACAAGGCAGTTGTAAG
AAATACATATATCTGAATAAATATTTGGGGGATGTTAAAAATATTAAAGTTATCTATGGACCTGCAGCTCGAT
TGAGACCCTCTGATGTCCCAGATAAATACTATTCATTTAACTATGAAGGCATTGCCCGAAATCTTTCTTGCCG
GGAACCAAACCAGCACTTCAAACCTTACCTGAAACATTTCTTACCTAAGCGTTTGCACTTTGCTAAGAGTGAT
AGAATTGAGCCCTTGACATTCTATTTGGACCCTCAGTGGCAACTTGCATTGAATCCCTCAGAAAGGAAATATT
GTGGAAGTGGATTTCATGGCTCTGACAATGTATTTTCAAATATGCAAGCCCTCTTTGTTGGCTATGGACCTGG
ATTCAAGCATGGCATTGAGGCTGACACCTTTGAAAACATTGAAGTCTATAACTTAATGTGTGATTTACTGAAT
TTGACACCGGCTCCTAATAACGGAACTCATGGAAGTCTTAACCACCTTCTAAAGAATCCTGTTTATACGCCAA
AGCATCCCAAAGAAGTGCACCCCCTGGTACAGTGCCCCTTCACAAGAAACCCCAGAGATAACCTTGGCTGCTC
ATGTAACCCTTCGATTTTGCCGATTGAGGATTTTCAAACACAGTTCAATCTGACTGTGGCAGAAGAGAAGATT
ATTAAGCATGAAACTTTACCCTATGGAAGACCTAGAGTTCTCCAGAAGGAAAACACCATCTGTCTTCTTTCCC
AGCACCAGTTTATGAGTGGATACAGCCAAGACATCTTAATGCCCCTTTGGACATCCTATACCGTGGACAGAAA
TGACAGTTTCTCTACGGAAGACTTCTCCAACTGTCTGTACCAGGACTTTAGAATTCCTCTTAGTCCTGTCCAT
AAATGTTCATTTTATAAAAATAACACCAAAGTGAGTTACGGGTTCCTCTCCCCACCACAACTAAATAAAAATT
CAAGTGGAATATATTCTGAAGCTTTGCTTACTACAAATATAGTGCCAATGTACCAGAGTTTTCAAGTTATATG
GCGCTACTTTCATGACACCCTACTGCGAAAGTATGCTGAAGAAAGAAATGGTGTCAATGTCGTCAGTGGTCCT
GTGTTTGACTTTGATTATGATGGACGTTGTGATTCCTTAGAGAATCTGAGGCAAAAAAGAAGAGTCATCCGTA
ACCAAGAAATTTTGATTCCAACTCACTTCTTTATTGTGCTAACAAGCTGTAAAGATACATCTCAGACGCCTTT
GCACTGTGAAAACCTAGACACCTTAGCTTTCATTTTGCCTCACAGGACTGATAACAGCGAGAGCTGTGTGCAT
GGGAAGCATGACTCCTCATGGGTTGAAGAATTGTTAATGTTACACAGAGCACGGATCACAGATGTTGAGCACA
TCACTGGACTCAGCTTCTATCAACAAAGAAAAGAGCCAGTTTCAGACATTTTAAAGTTGAAAACACATTTGCC
AACCTTTAGCCAAGAAGACGACTACAAAGACGATGACGACAAGTGAGGTACCGAGCTCGGATCCAAACAAACA
CCATTGTCACACTCCAACAAACACCATTGTCACACTCCAACAAACACCATTGTCACACTCCATTCTCGAGTCT
AGAAAGAGATCCAGACATGATAAGATACATTGATGAGTTTGGACAAACCACAACTAGAATGCAGTGAAAAAAA
TGCTTTATTTGTGAAATTTGTGATGCTATTGCTTTATTTGTAACCATTATAAGCTGCAATAAACAAGTTAACA
ACAACAATTGCATTCATTTTATGTTTCAGGTTCAGGGGGAGGTGTGGGAGGTTTTTTAGTCGACTAGAGCTCG
CTGATCAGCCTCGACTGTGCCTTCTAGTTGCCAGCCATCTGTTGTTTGCCCCTCCCCCGTGCCTTCCTTGACC
CTGGAAGGTGCCACTCCCACTGTCCTTTCCTAATAAAATGAGGAAATTGCATCGCATTGTCTGAGTAGGTGTC
ATTCTATTCTGGGGGGTGGGGTGGGGCAGGACAGCAAGGGGGAGGATTGGGAAGACAATAGCAGGCATGCTGG
GGAGAGATCTGAGGAACCCCTAGTGATGGAGTTGGCCACTCCCTCTCTGCGCGCTCGCTCGCTCACTGAGGCC
GGGCGACCAAAGGTCGCCCGACGCCCGGGCTTTGCCCGGGCGGCCTCAGTGAGCGAGCGAGCGCGCAGAGAGG
GAGTGGCCCCCCCCCCCCCCCCCCCTGCAGCCTGCATTAATGAATCGGCCAACGCGCGGGGAGAGGCGGTTTG
CGTATTGGGCGCTCTTCCGCTTCCTCGCTCACTGACTCGCTGCGCTCGGTCGTTCGGCTGCGGCGAGCGGTAT
CAGCTCACTCAAAGGCGGTAATACGGTTATCCACAGAATCAGGGGATAACGCAGGAAAGAACATGTGAGCAAA
AGGCCAGCAAAAGGCCAGGAACCGTAAAAAGGCCGCGTTGCTGGCGTTTTTCCATAGGCTCCGCCCCCCTGAC
GAGCATCACAAAAATCGACGCTCAAGTCAGAGGTGGCGAAACCCGACAGGACTATAAAGATACCAGGCGTTTC
CCCCTGGAAGCTCCCTCGTGCGCTCTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGTTCGGTGTAGGTCGTTCGCTCCAAG
CTGGGCTGTGTGCACGAACCCCCCGTTCAGCCCGACCGCTGCGCCTTATCCGGTAACTATCGTCTTGAGTCCA
ACCCGGTAAGACACGACTTATCGCCACTGGCAGCAGCCACTGGTAACAGGATTAGCAGAGCGAGGTATGTAGG
CGGTGCTACAGAGTTCTTGAAGTGGTGGCCTAACTACGGCTACACTAGAAGAACAGTATTTGGTATCTGCGCT
CTGCTGAAGCCAGTTACCTTCGGAAAAAGAGTTGGTAGCTCTTGATCCGGCAAACAAACCACCGCTGGTAGCG
GTGGTTTTTTTGTTTGCAAGCAGCAGATTACGCGCAGAAAAAAAGGATCTCAAGAAGATCCTTTGATCTTTTC
TACGGGGTCTGACGCTCAGTGGAACGAAAACTCACGTTAAGGGATTTTGGTCATGAGATTATCAAAAAGGATC
TTCACCTAGATCCTTTTAAATTAAAAATGAAGTTTTAAATCAATCTAAAGTATATATGAGTAAACTTGGTCTG
ACAGTTACCAATGCTTAATCAGTGAGGCACCTATCTCAGCGATCTGTCTATTTCGTTCATCCATAGTTGCCTG
ACTCCCCGTCGTGTAGATAACTACGATACGGGAGGGCTTACCATCTGGCCCCAGTGCTGCAATGATACCGCGA
GACCCACGCTCACCGGCTCCAGATTTATCAGCAATAAACCAGCCAGCCGGAAGGGCCGAGCGCAGAAGTGGTC
CTGCAACTTTATCCGCCTCCATCCAGTCTATTAATTGTTGCCGGGAAGCTAGAGTAAGTAGTTCGCCAGTTAA
TAGTTTGCGCAACGTTGTTGCCATTGCTACAGGCATCGTGGTGTCACGCTCGTCGTTTGGTATGGCTTCATTC
AGCTCCGGTTCCCAACGATCAAGGCGAGTTACATGATCCCCCATGTTGTGCAAAAAAGCGGTTAGCTCCTTCG
GTCCTCCGATCGTTGTCAGAAGTAAGTTGGCCGCAGTGTTATCACTCATGGTTATGGCAGCACTGCATAATTC
TCTTACTGTCATGCCATCCGTAAGATGCTTTTCTGTGACTGGTGAGTACTCAA
pAAV-ENPP1-FL-miR155 3x BS
(SEQ ID NO: 102)
CCAAGTCATTCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATAAGGGCGACACGGAAATGTTG
AATACTCATACTCTTCCTTTTTCAATATTATTGAAGCATTTATCAGGGTTATTGTCTCATGAGCGGATACATA
TTTGAATGTATTTAGAAAAATAAACAAATAGGGGTTCCGCGCACATTTCCCCGAAAAGTGCCACCTGACGTCT
AAGAAACCATTATTATCATGACATTAACCTATAAAAATAGGCGTATCACGAGGCCCTTTCGTCTCGCGCGTTT
CGGTGATGACGGTGAAAACCTCTGACACATGCAGCTCCCGGAGACGGTCACAGCTTGTCTGTAAGCGGATGCC
GGGAGCAGACAAGCCCGTCAGGGCGCGTCAGCGGGTGTTGGCGGGTGTCGGGGCTGGCTTAACTATGCGGCAT
CAGAGCAGATTGTACTGAGAGTGCACCATATGCGGTGTGAAATACCGCACAGATGCGTAAGGAGAAAATACCG
CATCAGGAAATTGTAAGCGTTAATATTTTGTTAAAATTCGCGTTAAATTTTTGTTAAATCAGCTCATTTTTTA
ACCAATAGGCCGAAATCGGCAAAATCCCTTATAAATCAAAAGAATAGACCGAGATAGGGTTGAGTGTTGTTCC
AGTTTGGAACAAGAGTCCACTATTAAAGAACGTGGACTCCAACGTCAAAGGGCGAAAAACCGTCTATCAGGGC
GATGGCCCACTACGTGAACCATCACCCTAATCAAGTTTTTTGGGGTCGAGGTGCCGTAAAGCACTAAATCGGA
ACCCTAAAGGGAGCCCCCGATTTAGAGCTTGACGGGGAAAGCCGGCGAACGTGGCGAGAAAGGAAGGGAAGAA
AGCGAAAGGAGCGGGCGCTAGGGCGCTGGCAAGTGTAGCGGTCACGCTGCGCGTAACCACCACACCCGCCGCG
CTTAATGCGCCGCTACAGGGCGCGTCCATTCGCCATTCAGGCTGCGCAACTGTTGGGAAGGGCGATCGGTGCG
GGCCTCTTCGCTATTACGCCAGGCTGCAGGGGGGGGGGGGGGGGGGCCACTCCCTCTCTGCGCGCTCGCTCGC
TCACTGAGGCCGCCCGGGCAAAGCCCGGGCGTCGGGCGACCTTTGGTCGCCCGGCCTCAGTGAGCGAGCGAGC
GCGCAGAGAGGGAGTGGCCAACTCCATCACTAGGGGTTCCTCAGATCTGAATTCGGTACCTAGTTATTAATAG
TAATCAATTACGGGGTCATTAGTTCATAGCCCATATATGGAGTTCCGCGTTACATAACTTACGGTAAATGGCC
CGCCTGGCTGACCGCCCAACGACCCCCGCCCATTGACGTCAATAATGACGTATGTTCCCATAGTAACGCCAAT
AGGGACTTTCCATTGACGTCAATGGGTGGAGTATTTACGGTAAACTGCCCACTTGGCAGTACATCAAGTGTAT
CATATGCCAAGTACGCCCCCTATTGACGTCAATGACGGTAAATGGCCCGCCTGGCATTATGCCCAGTACATGA
CCTTATGGGACTTTCCTACTTGGCAGTACATCTACGTATTAGTCATCGCTATTACCATGGTCGAGGTGAGCCC
CACGTTCTGCTTCACTCTCCCCATCTCCCCCCCCTCCCCACCCCCAATTTTGTATTTATTTATTTTTTAATTA
TTTTGTGCAGCGATGGGGGCGGGGGGGGGGGGGGGGCGCGCGCCAGGCGGGGGGGGCGGGGCGAGGGGGGGG
GCGGGGCGAGGCGGAGAGGTGCGGCGGCAGCCAATCAGAGCGGCGCGCTCCGAAAGTTTCCTTTTATGGCGAG
GCGGCGGCGGCGGCGGCCCTATAAAAAGCGAAGCGCGCGGCGGGGGGAGTCGCTGCGCGCTGCCTTCGCCCC
GTGCCCCGCTCCGCCGCCGCCTCGCGCCGCCCGCCCCGGCTCTGACTGACCGCGTTACTCCCACAGGTGAGCG
GGCGGGACGGCCCTTCTCCTCCGGGCTGTAATTAGCGCTTGGTTTAATGACGGCTTGTTTCTTTTCTGTGGCT
GCGTGAAAGCCTTGAGGGGCTCCGGGAGCTAGAGCCTCTGCTAACCATGTTCATGCCTTCTTCTTTTTCCTAC
AGCTCCTGGGCAACGTGCTGGTTATTGTGCTGTCTCATCATTTTGGCAAAGAATTCCTCGAAGATCCGAAGGG
GTTCAAGCTTAGCGCCACCATGGAGCGCGACGGCTGCGCGGGGGGCGGGAGCCGCGGCGGCGAGGGCGGGCGC
GCTCCCCGGGAGGGCCCGGCGGGGAACGGCCGCGATCGGGGCCGCAGCCACGCTGCCGAGGCGCCCGGGGACC
CGCAGGCCGCGGCGTCCTTGCTGGCCCCTATGGACGTGGGGGAGGAGCCGCTGGAGAAGGCGGCGCGCGCCCG
CACTGCCAAGGACCCCAACACCTATAAAGTACTCTCGCTGGTATTGTCAGTATGTGTGTTAACAACAATACTT
GGTTGTATATTTGGGTTGAAACCAAGCTGTGCCAAAGAAGTTAAAAGTTGCAAAGGTCGCTGTTTCGAGAGAA
CATTTGGGAACTGTCGCTGTGATGCTGCCTGTGTTGAGCTTGGAAACTGCTGTTTAGATTACCAGGAGACGTG
CATAGAACCAGAACATATATGGACTTGCAACAAATTCAGGTGTGGTGAGAAAAGGTTGACCAGAAGCCTCTGT
GCCTGTTCAGATGACTGCAAGGACAAGGGCGACTGCTGCATCAACTACAGTTCTGTGTGTCAAGGTGAGAAAA
GTTGGGTAGAAGAACCATGTGAGAGCATTAATGAGCCACAGTGCCCAGCAGGGTTTGAAACGCCTCCTACCCT
CTTATTTTCTTTGGATGGATTCAGGGCAGAATATTTACACACTTGGGGTGGACTTCTTCCTGTTATTAGCAAA
CTAAAAAAATGTGGAACATATACTAAAAACATGAGACCGGTATATCCAACAAAAACTTTCCCCAATCACTACA
GCATTGTCACCGGATTGTATCCAGAATCTCATGGCATAATCGACAATAAAATGTATGATCCCAAAATGAATGC
TTCCTTTTCACTTAAAAGTAAAGAGAAATTTAATCCTGAGTGGTACAAAGGAGAACCAATTTGGGTCACAGCT
AAGTATCAAGGCCTCAAGTCTGGCACATTTTTCTGGCCAGGATCAGATGTGGAAATTAACGGAATTTTCCCAG
ACATCTATAAAATGTATAATGGTTCAGTACCATTTGAAGAAAGGATTTTAGCTGTTCTTCAGTGGCTACAGCT
TCCTAAAGATGAAAGACCACACTTTTACACTCTGTATTTAGAAGAACCAGATTCTTCAGGTCATTCATATGGA
CCAGTCAGCAGTGAAGTCATCAAAGCCTTGCAGAGGGTTGATGGTATGGTTGGTATGCTGATGGATGGTCTGA
AAGAGCTGAACTTGCACAGATGCCTGAACCTCATCCTTATTTCAGATCATGGCATGGAACAAGGCAGTTGTAA
GAAATACATATATCTGAATAAATATTTGGGGGATGTTAAAAATATTAAAGTTATCTATGGACCTGCAGCTCGA
TTGAGACCCTCTGATGTCCCAGATAAATACTATTCATTTAACTATGAAGGCATTGCCCGAAATCTTTCTTGCC
GGGAACCAAACCAGCACTTCAAACCTTACCTGAAACATTTCTTACCTAAGCGTTTGCACTTTGCTAAGAGTGA
TAGAATTGAGCCCTTGACATTCTATTTGGACCCTCAGTGGCAACTTGCATTGAATCCCTCAGAAAGGAAATAT
TGTGGAAGTGGATTTCATGGCTCTGACAATGTATTTTCAAATATGCAAGCCCTCTTTGTTGGCTATGGACCTG
GATTCAAGCATGGCATTGAGGCTGACACCTTTGAAAACATTGAAGTCTATAACTTAATGTGTGATTTACTGAA
TTTGACACCGGCTCCTAATAACGGAACTCATGGAAGTCTTAACCACCTTCTAAAGAATCCTGTTTATACGCCA
AAGCATCCCAAAGAAGTGCACCCCCTGGTACAGTGCCCCTTCACAAGAAACCCCAGAGATAACCTTGGCTGCT
CATGTAACCCTTCGATTTTGCCGATTGAGGATTTTCAAACACAGTTCAATCTGACTGTGGCAGAAGAGAAGAT
TATTAAGCATGAAACTTTACCCTATGGAAGACCTAGAGTTCTCCAGAAGGAAAACACCATCTGTCTTCTTTCC
CAGCACCAGTTTATGAGTGGATACAGCCAAGACATCTTAATGCCCCTTTGGACATCCTATACCGTGGACAGAA
ATGACAGTTTCTCTACGGAAGACTTCTCCAACTGTCTGTACCAGGACTTTAGAATTCCTCTTAGTCCTGTCCA
TAAATGTTCATTTTATAAAAATAACACCAAAGTGAGTTACGGGTTCCTCTCCCCACCACAACTAAATAAAAAT
TCAAGTGGAATATATTCTGAAGCTTTGCTTACTACAAATATAGTGCCAATGTACCAGAGTTTTCAAGTTATAT
GGCGCTACTTTCATGACACCCTACTGCGAAAGTATGCTGAAGAAAGAAATGGTGTCAATGTCGTCAGTGGTCC
TGTGTTTGACTTTGATTATGATGGACGTTGTGATTCCTTAGAGAATCTGAGGCAAAAAAGAAGAGTCATCCGT
AACCAAGAAATTTTGATTCCAACTCACTTCTTTATTGTGCTAACAAGCTGTAAAGATACATCTCAGACGCCTT
TGCACTGTGAAAACCTAGACACCTTAGCTTTCATTTTGCCTCACAGGACTGATAACAGCGAGAGCTGTGTGCA
TGGGAAGCATGACTCCTCATGGGTTGAAGAATTGTTAATGTTACACAGAGCACGGATCACAGATGTTGAGCAC
ATCACTGGACTCAGCTTCTATCAACAAAGAAAAGAGCCAGTTTCAGACATTTTAAAGTTGAAAACACATTTGC
CAACCTTTAGCCAAGAAGACGACTACAAAGACGATGACGACAAGTGAGGTACCGAGCTCGGATCCCAATTACG
ATTAGCACTATCCAATTACGATTAGCACTATCCAATTACGATTAGCACTATCCTCGAGTCTAGAAAGAGATCC
AGACATGATAAGATACATTGATGAGTTTGGACAAACCACAACTAGAATGCAGTGAAAAAAATGCTTTATTTGT
GAAATTTGTGATGCTATTGCTTTATTTGTAACCATTATAAGCTGCAATAAACAAGTTAACAACAACAATTGCA
TTCATTTTATGTTTCAGGTTCAGGGGGAGGTGTGGGAGGTTTTTTAGTCGACTAGAGCTCGCTGATCAGCCTC
GACTGTGCCTTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATTCTATTCTGG
GGGGTGGGGTGGGGCAGGACAGCAAGGGGGAGGATTGGGAAGACAATAGCAGGCATGCTGGGGAGAGATCTGA
GGAACCCCTAGTGATGGAGTTGGCCACTCCCTCTCTGCGCGCTCGCTCGCTCACTGAGGCCGGGCGACCAAAG
GTCGCCCGACGCCCGGGCTTTGCCCGGGCGGCCTCAGTGAGCGAGCGAGCGCGCAGAGAGGGAGTGGCCCCCC
CCCCCCCCCCCCCTGCAGCCTGCATTAATGAATCGGCCAACGCGCGGGGAGAGGCGGTTTGCGTATTGGGCGC
TCTTCCGCTTCCTCGCTCACTGACTCGCTGCGCTCGGTCGTTCGGCTGCGGCGAGCGGTATCAGCTCACTCAA
AGGCGGTAATACGGTTATCCACAGAATCAGGGGATAACGCAGGAAAGAACATGTGAGCAAAAGGCCAGCAAAA
GGCCAGGAACCGTAAAAAGGCCGCGTTGCTGGCGTTTTTCCATAGGCTCCGCCCCCCTGACGAGCATCACAAA
AATCGACGCTCAAGTCAGAGGTGGCGAAACCCGACAGGACTATAAAGATACCAGGCGTTTCCCCCTGGAAGCT
CCCTCGTGCGCTCTCCTGTTCCGACCCTGCCGCTTACCGGATACCTGTCCGCCTTTCTCCCTTCGGGAAGCGT
GGCGCTTTCTCATAGCTCACGCTGTAGGTATCTCAGTTCGGTGTAGGTCGTTCGCTCCAAGCTGGGCTGTGTG
CACGAACCCCCCGTTCAGCCCGACCGCTGCGCCTTATCCGGTAACTATCGTCTTGAGTCCAACCCGGTAAGAC
ACGACTTATCGCCACTGGCAGCAGCCACTGGTAACAGGATTAGCAGAGCGAGGTATGTAGGCGGTGCTACAGA
GTTCTTGAAGTGGTGGCCTAACTACGGCTACACTAGAAGAACAGTATTTGGTATCTGCGCTCTGCTGAAGCCA
GTTACCTTCGGAAAAAGAGTTGGTAGCTCTTGATCCGGCAAACAAACCACCGCTGGTAGCGGTGGTTTTTTTG
TTTGCAAGCAGCAGATTACGCGCAGAAAAAAAGGATCTCAAGAAGATCCTTTGATCTTTTCTACGGGGTCTGA
CGCTCAGTGGAACGAAAACTCACGTTAAGGGATTTTGGTCATGAGATTATCAAAAAGGATCTTCACCTAGATC
CTTTTAAATTAAAAATGAAGTTTTAAATCAATCTAAAGTATATATGAGTAAACTTGGTCTGACAGTTACCAAT
GCTTAATCAGTGAGGCACCTATCTCAGCGATCTGTCTATTTCGTTCATCCATAGTTGCCTGACTCCCCGTCGT
GTAGATAACTACGATACGGGAGGGCTTACCATCTGGCCCCAGTGCTGCAATGATACCGCGAGACCCACGCTCA
CCGGCTCCAGATTTATCAGCAATAAACCAGCCAGCCGGAAGGGCCGAGCGCAGAAGTGGTCCTGCAACTTTAT
CCGCCTCCATCCAGTCTATTAATTGTTGCCGGGAAGCTAGAGTAAGTAGTTCGCCAGTTAATAGTTTGCGCAA
CGTTGTTGCCATTGCTACAGGCATCGTGGTGTCACGCTCGTCGTTTGGTATGGCTTCATTCAGCTCCGGTTCC
CAACGATCAAGGCGAGTTACATGATCCCCCATGTTGTGCAAAAAAGCGGTTAGCTCCTTCGGTCCTCCGATCG
TTGTCAGAAGTAAGTTGGCCGCAGTGTTATCACTCATGGTTATGGCAGCACTGCATAATTCTCTTACTGTCAT
GCCATCCGTAAGATGCTTTTCTGTGACTGGTGAGTACTCAA
pAAV-CAG-ENPP1 FL
(SEQ ID NO: 103)
tcgactagagctcgctgatcagcctcgactgtgccttctagttgccagccatctgttgtttgcccctcccccg
tgccttccttgaccctggaaggtgccactcccactgtcctttcctaataaaatgaggaaattgcatcgcattg
tctgagtaggtgtcattctattctggggggtggggtggggcaggacagcaagggggaggattgggaagacaat
agcaggcatgctggggagagatctgaggaacccctagtgatggagttggccactccctctctgcgcgctcgct
cgctcactgaggccgggcgaccaaaggtcgcccgacgcccgggctttgcccgggcggcctcagtgagcgagcg
agcgcgcagagagggagtggcccccccccccccccctgcagcctgcattaatgaatcggccaacgcgcgggga
gaggcggtttgcgtattgggcgctcttccgcttcctcgctcactgactcgctgcgctcggtcgttcggctgcg
gcgagcggtatcagctcactcaaaggcggtaatacggttatccacagaatcaggggataacgcaggaaagaac
atgtgagcaaaaggccagcaaaaggccaggaaccgtaaaaaggccgcgttgctggcgtttttccataggctcc
gcccccctgacgagcatcacaaaaatcgacgctcaagtcagaggtggcgaaacccgacaggactataaagata
ccaggcgtttccccctggaagctccctcgtgcgctctcctgttccgaccctgccgcttaccggatacctgtcc
gcctttctcccttcgggaagcgtggcgctttctcatagctcacgctgtaggtatctcagttcggtgtaggtcg
ttcgctccaagctgggctgtgtgcacgaaccccccgttcagcccgaccgctgcgccttatccggtaactatcg
tcttgagtccaacccggtaagacacgacttatcgccactggcagcagccactggtaacaggattagcagagcg
aggtatgtaggcggtgctacagagttcttgaagtggtggcctaactacggctacactagaagaacagtatttg
gtatctgcgctctgctgaagccagttaccttcggaaaaagagttggtagctcttgatccggcaaacaaaccac
cgctggtagcggtggtttttttgtttgcaagcagcagattacgcgcagaaaaaaaggatctcaagaagatcct
ttgatcttttctacggggtctgacgctcagtggaacgaaaactcacgttaagggattttggtcatgagattat
caaaaaggatcttcacctagatccttttaaattaaaaatgaagttttaaatcaatctaaagtatatatgagta
aacttggtctgacagttaccaatgcttaatcagtgaggcacctatctcagcgatctgtctatttcgttcatcc
atagttgcctgactccccgtcgtgtagataactacgatacgggagggcttaccatctggccccagtgctgcaa
tgataccgcgagacccacgctcaccggctccagatttatcagcaataaaccagccagccggaagggccgagcg
cagaagtggtcctgcaactttatccgcctccatccagtctattaattgttgccgggaagctagagtaagtagt
tcgccagttaatagtttgcgcaacgttgttgccattgctacaggcatcgtggtgtcacgctcgtcgtttggta
tggcttcattcagctccggttcccaacgatcaaggcgagttacatgatcccccatgttgtgcaaaaaagcggt
tagctccttcggtcctccgatcgttgtcagaagtaagttggccgcagtgttatcactcatggttatggcagca
ctgcataattctcttactgtcatgccatccgtaagatgcttttctgtgactggtgagtactcaaccaagtcat
tctgagaatagtgtatgcggcgaccgagttgctcttgcccggcgtcaatacgggataataccgcgccacatag
cagaactttaaaagtgctcatcattggaaaacgttcttcggggcgaaaactctcaaggatcttaccgctgttg
agatccagttcgatgtaacccactcgtgcacccaactgatcttcagcatcttttactttcaccagcgtttctg
ggtgagcaaaaacaggaaggcaaaatgccgcaaaaaagggaataagggcgacacggaaatgttgaatactcat
actcttcctttttcaatattattgaagcatttatcagggttattgtctcatgagcggatacatatttgaatgt
atttagaaaaataaacaaataggggttccgcgcacatttccccgaaaagtgccacctgacgtctaagaaacca
ttattatcatgacattaacctataaaaataggcgtatcacgaggccctttcgtctcgcgcgtttcggtgatga
cggtgaaaacctctgacacatgcagctcccggagacggtcacagcttgtctgtaagcggatgccgggagcaga
caagcccgtcagggcgcgtcagcgggtgttggcgggtgtcggggctggcttaactatgcggcatcagagcaga
ttgtactgagagtgcaccatatgcggtgtgaaataccgcacagatgcgtaaggagaaaataccgcatcaggaa
attgtaagcgttaatattttgttaaaattcgcgttaaatttttgttaaatcagctcattttttaaccaatagg
ccgaaatcggcaaaatcccttataaatcaaaagaatagaccgagatagggttgagtgttgttccagtttggaa
caagagtccactattaaagaacgtggactccaacgtcaaagggcgaaaaaccgtctatcagggcgatggccca
ctacgtgaaccatcaccctaatcaagttttttggggtcgaggtgccgtaaagcactaaatcggaaccctaaag
ggagcccccgatttagagcttgacggggaaagccggcgaacgtggcgagaaaggaagggaagaaagcgaaagg
agcgggcgctagggcgctggcaagtgtagcggtcacgctgcgcgtaaccaccacacccgccgcgcttaatgcg
ccgctacagggcgcgtccattcgccattcaggctgcgcaactgttgggaagggcgatcggtgcgggcctcttc
gctattacgccaggctgcaggggggggggggggggccactccctctctgcgcgctcgctcgctcactgaggcc
gcccgggcaaagcccgggcgtcgggcgacctttggtcgcccggcctcagtgagcgagcgagcgcgcagagagg
gagtggccaactccatcactaggggttcctcagatctgaattcggtacctagttattaatagtaatcaattac
ggggtcattagttcatagcccatatatggagttccgcgttacataacttacggtaaatggcccgcctggctga
ccgcccaacgacccccgcccattgacgtcaataatgacgtatgttcccatagtaacgccaatagggactttcc
attgacgtcaatgggtggagtatttacggtaaactgcccacttggcagtacatcaagtgtatcatatgccaag
tacgccccctattgacgtcaatgacggtaaatggcccgcctggcattatgcccagtacatgaccttatgggac
tttcctacttggcagtacatctacgtattagtcatcgctattaccatggtcgaggtgagccccacgttctgct
tcactctccccatctcccccccctccccacccccaattttgtatttatttattttttaattattttgtgcagc
gatgggggcggggggggggggggggcgcgcgccaggcggggcggggcggggcgaggggggggcggggcgagg
cggagaggtgcggcggcagccaatcagagcggcgcgctccgaaagtttccttttatggcgaggcggcggcggc
ggcggccctataaaaagcgaagcgcgcggcgggcgggagtcgctgcgcgctgccttcgccccgtgccccgctc
cgccgccgcctcgcgccgcccgccccggctctgactgaccgcgttactcccacaggtgagcgggcgggacggc
ccttctcctccgggctgtaattagcgcttggtttaatgacggcttgtttcttttctgtggctgcgtgaaagcc
ttgaggggctccgggagctagagcctctgctaaccatgttcatgccttcttctttttcctacagctcctgggc
aacgtgctggttattgtgctgtctcatcattttggcaaagaattcctcgaagatccgaaggggttcaagctCT
AGCGCCACCATGGAGCGCGACGGCTGCGCGGGGGGCGGGAGCCGCGGCGGCGAGGGCGGGCGCGCTCCCCGGG
AGGGCCCGGCGGGGAACGGCCGCGATCGGGGCCGCAGCCACGCTGCCGAGGCGCCCGGGGACCCGCAGGCCGC
GGCGTCCTTGCTGGCCCCTATGGACGTGGGGGAGGAGCCGCTGGAGAAGGCGGCGCGCGCCCGCACTGCCAAG
GACCCCAACACCTATAAAGTACTCTCGCTGGTATTGTCAGTATGTGTGTTAACAACAATACTTGGTTGTATAT
TTGGGTTGAAACCAAGCTGTGCCAAAGAAGTTAAAAGTTGCAAAGGTCGCTGTTTCGAGAGAACATTTGGGAA
CTGTCGCTGTGATGCTGCCTGTGTTGAGCTTGGAAACTGCTGTTTAGATTACCAGGAGACGTGCATAGAACCA
GAACATATATGGACTTGCAACAAATTCAGGTGTGGTGAGAAAAGGTTGACCAGAAGCCTCTGTGCCTGTTCAG
ATGACTGCAAGGACAAGGGCGACTGCTGCATCAACTACAGTTCTGTGTGTCAAGGTGAGAAAAGTTGGGTAGA
AGAACCATGTGAGAGCATTAATGAGCCACAGTGCCCAGCAGGGTTTGAAACGCCTCCTACCCTCTTATTTTCT
TTGGATGGATTCAGGGCAGAATATTTACACACTTGGGGTGGACTTCTTCCTGTTATTAGCAAACTAAAAAAAT
GTGGAACATATACTAAAAACATGAGACCGGTATATCCAACAAAAACTTTCCCCAATCACTACAGCATTGTCAC
CGGATTGTATCCAGAATCTCATGGCATAATCGACAATAAAATGTATGATCCCAAAATGAATGCTTCCTTTTCA
CTTAAAAGTAAAGAGAAATTTAATCCTGAGTGGTACAAAGGAGAACCAATTTGGGTCACAGCTAAGTATCAAG
GCCTCAAGTCTGGCACATTTTTCTGGCCAGGATCAGATGTGGAAATTAACGGAATTTTCCCAGACATCTATAA
AATGTATAATGGTTCAGTACCATTTGAAGAAAGGATTTTAGCTGTTCTTCAGTGGCTACAGCTTCCTAAAGAT
GAAAGACCACACTTTTACACTCTGTATTTAGAAGAACCAGATTCTTCAGGTCATTCATATGGACCAGTCAGCA
GTGAAGTCATCAAAGCCTTGCAGAGGGTTGATGGTATGGTTGGTATGCTGATGGATGGTCTGAAAGAGCTGAA
CTTGCACAGATGCCTGAACCTCATCCTTATTTCAGATCATGGCATGGAACAAGGCAGTTGTAAGAAATACATA
TATCTGAATAAATATTTGGGGGATGTTAAAAATATTAAAGTTATCTATGGACCTGCAGCTCGATTGAGACCCT
CTGATGTCCCAGATAAATACTATTCATTTAACTATGAAGGCATTGCCCGAAATCTTTCTTGCCGGGAACCAAA
CCAGCACTTCAAACCTTACCTGAAACATTTCTTACCTAAGCGTTTGCACTTTGCTAAGAGTGATAGAATTGAG
CCCTTGACATTCTATTTGGACCCTCAGTGGCAACTTGCATTGAATCCCTCAGAAAGGAAATATTGTGGAAGTG
GATTTCATGGCTCTGACAATGTATTTTCAAATATGCAAGCCCTCTTTGTTGGCTATGGACCTGGATTCAAGCA
TGGCATTGAGGCTGACACCTTTGAAAACATTGAAGTCTATAACTTAATGTGTGATTTACTGAATTTGACACCG
GCTCCTAATAACGGAACTCATGGAAGTCTTAACCACCTTCTAAAGAATCCTGTTTATACGCCAAAGCATCCCA
AAGAAGTGCACCCCCTGGTACAGTGCCCCTTCACAAGAAACCCCAGAGATAACCTTGGCTGCTCATGTAACCC
TTCGATTTTGCCGATTGAGGATTTTCAAACACAGTTCAATCTGACTGTGGCAGAAGAGAAGATTATTAAGCAT
GAAACTTTACCCTATGGAAGACCTAGAGTTCTCCAGAAGGAAAACACCATCTGTCTTCTTTCCCAGCACCAGT
TTATGAGTGGATACAGCCAAGACATCTTAATGCCCCTTTGGACATCCTATACCGTGGACAGAAATGACAGTTT
CTCTACGGAAGACTTCTCCAACTGTCTGTACCAGGACTTTAGAATTCCTCTTAGTCCTGTCCATAAATGTTCA
TTTTATAAAAATAACACCAAAGTGAGTTACGGGTTCCTCTCCCCACCACAACTAAATAAAAATTCAAGTGGAA
TATATTCTGAAGCTTTGCTTACTACAAATATAGTGCCAATGTACCAGAGTTTTCAAGTTATATGGCGCTACTT
TCATGACACCCTACTGCGAAAGTATGCTGAAGAAAGAAATGGTGTCAATGTCGTCAGTGGTCCTGTGTTTGAC
TTTGATTATGATGGACGTTGTGATTCCTTAGAGAATCTGAGGCAAAAAAGAAGAGTCATCCGTAACCAAGAAA
TTTTGATTCCAACTCACTTCTTTATTGTGCTAACAAGCTGTAAAGATACATCTCAGACGCCTTTGCACTGTGA
AAACCTAGACACCTTAGCTTTCATTTTGCCTCACAGGACTGATAACAGCGAGAGCTGTGTGCATGGGAAGCAT
GACTCCTCATGGGTTGAAGAATTGTTAATGTTACACAGAGCACGGATCACAGATGTTGAGCACATCACTGGAC
TCAGCTTCTATCAACAAAGAAAAGAGCCAGTTTCAGACATTTTAAAGTTGAAAACACATTTGCCAACCTTTAG
CCAAGAAGACGACTACAAAGACGATGACGACAAGTGAGGTACCGAGCTCGGATCCACTAGTCCAGTGTGGTGG
AATTCTGCAGATATCCAGCACAGTGGCGGCCGCTCGAGTctagaaagagatccagacatgataagatacattg
atgagtttggacaaaccacaactagaatgcagtgaaaaaaatgctttatttgtgaaatttgtgatgctattgc
tttatttgtaaccattataagctgcaataaacaagttaacaacaacaattgcattcattttatgtttcaggtt
cagggggaggtgtgggaggttttttag

### EXAMPLES

The invention is further described in the following examples, which do not limit the scope of the invention described in the claims.

### Materials and Methods

The following materials and methods were used in the Examples below.

### Chemicals and Antibodies

Chemicals used in the study were purchased either from Millipore Sigma, Invitrogen, or Thermofischer Scientific. Antibodies were purchased from Cell Signaling Technology (CST), Invitrogen or Abcam. A list of the antibodies used in the study is given below.

| Antibody | Catalog Number | Manufacturer |
|---|---|---|
| ENPP1 | 2061S | CST |
| DYKDDDDK tag (Flag) | 14793S, #ab205606 | CST, Abcam |
| F-Actin A-488 | #R37110 | Invitrogen |

### Cell Cultures

HepG2 and HEK293T cells were maintained in Dulbecco's modified eagle medium supplemented with 10% FBS added with penicillin-streptomycin solution. Cells were grown in 75 cm² culture flasks in an incubator maintained at 37° C. supplied with 5% CO₂. Cells were trypsinized and passaged when they are 90% confluent.

### Mouse Colony Management and Genotyping

C57BL/6J-Enpp1asj/GrsrJ (the ASJ mice) were ordered from the Jackson Laboratory. The heterozygous mice were breed together. The C57BL/6J-Enpp1asj/GrsrJ breeders are put on an acceleration diet (Envigo, TD.00442, 10 kg, pellet diet), all pups are maintained on the acceleration diet once they are weaned.

### Plasmids and Transfections

DNA plasmids were designed at MGH and synthesized at Gene script. Transfection was performed in a 6 well plate when the cells were at 60% confluence. Cells were sat in Optimem 24 hrs. before transfection. Then 2 ug of DNA plasmid per mL was mixed with either LTX lipofectamine at 5 ul/mL or Fugene at 6 ul/ml in Optimem. Transfection mixture was incubated for 15 min at room temperature followed by incubation with cells for 36 hrs. Then the transfecting medium was replaced by normal growth media and cells were collected 72 hrs. post transfection.

### Immunoprecipitation

Cell supernatant (media) added with protease and phosphatase inhibitor cocktail was incubated with anti-flag antibody overnight at 4° C. Protein A-agarose beads were blocked with 1% BSA in PBS for 1 hr at 4° C. followed by washings with PBS twice. Protein A-agarose beads were then added to cell supernatant solution and incubated for 1-3 hrs at 4° C. Beads were collected by centrifugation at 2500 rpm for 10 min at 4° C. Media was completely aspirated, and beads were resuspended in 20 ul of RIPA buffer added with protease and phosphatase inhibitor cocktail and heated at 95° C. by adding 2× NuPAGE LDS sample buffer. Samples were separated by SDS-PAGE and probed with either anti FLAG antibody or ENPP1 antibody.

### SDS-Polyacrylamide Gel Electrophoresis and Western Blotting

Cells were lysed using RIPA cell lysis buffer added with protease and phosphatase inhibitor cocktail (Thermo Fischer Scientific Cat #78440). Lysates were sonicated (3 cycles of 2 sec pulse on-15 sec pulse off) briefly on ice and were cleared by centrifugation at 12.5K rpm for 10 min at 4° C. Protein concentration was estimated using Pierce BCA protein assay kit per the manufacturer's instructions. 30 ug of lysates were prepared by NuPAGE LDS sample buffer and the samples were heated at 95° C. by placing the Eppendorf tubes on a heat block for 3-5 min. Protein samples were separated by sodium dodecyl sulfate-polyacrylamide gel electrophoresis (SDS-PAGE) followed by dry electro transfer onto a PVDF membrane using iblot2 transfer apparatus. Membranes were blocked with 5% BSA in Tris buffered saline (TBS) solution for 1 hr at room temperature followed by incubation with primary antibodies overnight. Membranes were washed with TBS-Tween-20 (0.05%) solution 5 times with an interval of 5 min. Blots were incubated with horse radish peroxidase (HRP)-tagged 2° antibodies (1:10000) at room temperature for 90 min followed by above mentioned TBS-Tween-20 washings. Blots were added with chemiluminescence substrates, and the signals were captured on a Bio-Rad gel doc imaging system and processed in Microsoft PowerPoint for presentation.

### ENPP1 Enzyme Activity Assay

Cells were seeded in 6-well plates and grown until confluent (10 days in DMEM follow by 5 days in αMEM). To measure ENPP1 enzyme activity, cells were lysed in 100 mM Tris-HCl (pH 9.0), 500 mM NaCl, 5 mM MgCl2, and 0.05% Triton X-100 and scraped into microcentrifuge tubes and kept on ice. Cell suspensions were spun at 12,000 g for 5 minutes. A 50 μl aliquot of the supernatant was added to a clear-bottom 96-well plate and the reaction with initiated upon adding 50 μl of 1 mM paranitrophenol-thymidine monophosphate (pNP-TMP; Sigma-Aldrich).

Mild Lysis Buffer [Store at 4° C.] pH 9.0 with 1 M NaOH

| | |
|---|---|
| 100 mM Tris-HCl | 20 mL of 1M Tris-HCl (pH 8.0) |
| 500 mM NaCl | 5.844 grams |
| 5 mM MgCl2 | 1 mL of 1M MgCl2 |
| | (4.76 gram in 50 mL diH2O) |
| 0.05% (v/v) Triton X-100 | 100 μl |
| diH2O | Fill up to 200 mL |

### Assay Reagents

4 mM pNP-TMP (Sigma T4510-100 mg, MW=465.28)
0.0186 g pNP-TMP in 10 mL of Mild Lysis Buffer
10 mM pNP solution (Sigma N7660-100 ml)

### Standard Curve

1. Make 1:10 dilution of 10 mM pNP to make final concentration of 1 mM 100 ul 10 mM pNP solution in 900 ul Mild Lysis Buffer
2. Prepare standard curve in duplicate (100 ul/well)

| | 0 | 2 | 4 | 6 | 8 | 10 | 20 | 30 | 40 | 50 |
|---|---|---|---|---|---|---|---|---|---|---|
| [ ] | nmol | nmol | nmol | nmol | nmol | nmol | nmol | nmol | nmol | nmol |
| 1 mM | 0 ul | 8 ul | 16 ul | 24 ul | 32 ul | 40 ul | 80 ul | 120 ul | 160 ul | 200 ul |
| standard | | | | | | | | | | |
| buffer | 400 ul | 392 ul | 384 ul | 376 ul | 368 ul | 360 ul | 320 ul | 280 ul | 240 ul | 200 ul |

For rENPP1 Activity Assay from Cell Lysates
1. Aspirate media from 6-well plates
2. Wash cells 2× with cold 1×PBS and aspirate wash solution
4. Lyse the cells (275 ul/well) with Mild Lysis Buffer
5. Scrape down cells with rubber policeman
6. Pipette into Eppendorf tubes and incubate on ice for 5 mins
7. Spin down cell debris at 12,000×g for 5 mins at 4° C.
8. Keep cell lysates on ice
9. Add 50 ul of sample/well (in triplicate) in 96-well plate
10. Initiate reaction by adding 50 ul of pNP-TMP substrate (with multi-channel pipette)
11. Incubate for 5, 10, 15, 20, 30 mins at 37° C. and read at 405 nm
12. Stop incubation when the absorbance of the samples reaches the range of the standards
13. Report data as nmol pNP produced/min/mg protein
14. Perform BCA to determine protein concentration during incubation; make BCA standards with Mild Lysis Buffer. Use 10 μl of standard or sample; 200 μl of WR reagent/well. A: 14 mL, B: 280 μl
15. Save remainder of protein lysate for Western blot (store at −80° C.)
   For srENPP1 Activity Assay from Cell Supernatant (Media)
1. Collect media from 6 well plates.
2. Add 50 ul of sample/well (in triplicate) in 96-well plate.
3. Initiate reaction by adding 50 ul of pNP-TMP substrate (with multi-channel pipette)
4. Incubate for 5, 10, 15, 20, 30 mins at 37° C. and read at 405 nm.
5. Stop incubation when the absorbance of the samples reaches the range of the standards.
6. Report data as nmol pNP produced/min/mg protein.
7. Perform BCA to determine protein concentration during incubation; make BCA standards with Mild Lysis Buffer. Use 10 μl of standard or sample; 200 μl of WR reagent/well. A: 14 mL, B: 280 μl

### Retroorbital Injections

The mouse was placed in a Plexiglas chamber and isoflurane was used for induction. Once adequate anesthesia was achieved, the mouse was placed in a left lateral position and gentle pressure is applied around the eye socket to partially protrude the eyeball. A 30 gauge, 0.5-in insulin needle was inserted bevel down into the retro-orbital sinus at the medial canthus and the solution was injected slowly. The needle was withdrawn smoothly, and the mouse was allowed to recover in its cage (JoVE Science Education Database. Lab Animal Research—Compound Administration IV. JoVE, Cambridge, MA, (2024)).

### Mouse Tissue Harvest and Cryosectioning

Mice of the Asj+/+ genotype (or other genotypes) were subjected to treatment with nanoparticles containing plasmid at 3 days and then harvested for a biodistribution study at 10 days. Serum/Plasma were obtained via cardiac puncture from anesthetized mice. Various tissues, including brain, heart, lungs, spleen, liver, kidneys, large intestine, bladder, tail, aorta, small intestine, knee, foot, and vertebrae, were harvested after brief perfusion with cold PBS. These tissues were promptly placed in optimal cutting temperature (OCT) compound for frozen sectioning. Cryosectioning was performed in a Leica CM 1850 cryostat and tissue sections of 10 um were cut and collected on to slides for immunofluorescence staining.

### Immunofluorescence

The tissue sections were washed with PBS twice to remove OCT and fixed with 4% paraformaldehyde (PFA) for 3 minutes at 4° C. followed by treatment with 0.1% Triton-X-100 for 5 min. The tissue slides were incubated in a 10% new donkey serum blocking solution. Primary antibodies used in the immunostaining included a recombinant Anti-DDDDK tag antibody (#ab205606, Abcam) and F-actin-A488 (#R37110, Invitrogen) was used for staining actin filaments. Alexa Fluor 594-conjugated secondary antibody was purchased from Jackson Immuno Research. Nuclei were stained with DAPI (#ab104139, Abcam). All the antibodies used in this study were validated for the species and applications by the indicated manufacturers. Confocal images were acquired using a Leica TCS SP8 X Laser Confocal Microscope (Leica). LAS X software (Leica) was used for acquisition and processing of images. Mean intensity was calculated using LAS X software and the values were plotted in Graph pad PRISM.

### Lipid Nanoparticles—Formulation and Peptide Conjugation

LNPs were formulated by pipette mixing a stock of organic phase composed of a lipid mixture with an aqueous phase composed of mRNA dissolved in 10 mM sodium acetate buffer, pH 5.2 (Sigma, 567422) at a volume ratio of 1:3 organic to aqueous. DLin-MC3-DMA (MC3) (Eschelone Bioscience, N-1282) Ionizable lipid, 1,2-dioleoyl-sn-glycero-3-phosphoethanolamine (DOPE, Avanti Polar Lipids, 850725P), cholesterol (Sigma, C8667) and 1,2-dimyristoyl-rac-glycero-3-methoxypolyethylene glycol-2000 (DMG-PEG 2000, Avanti Polar Lipids, 880151P) were dissolved in ethanol and mixed at predetermined molar ratios (50:10.5:38:1.5) and a 40:1 total lipids to mRNA weight ratio. The assembled LNPs were dialyzed against PBS (pH 7.4) in dialysis tubes (3500 MW cutoff, Sigma, PURD35050) to remove the ethanol. In formulations where 1,2-dioleoyl-3-trimethylammonium-propane (chloride salt) (DOTAP) was incorporated, the percentage of DOTAP was considered as part of the total 100% while the ratios between the remaining lipids was kept (50:10.5:38:1.5). Peptide-conjugated LNPs were composed of 10% DOTAP and DSPE-PEG 2000 (1,2-distearoyl-sn-glycero-3-phosphoethanolamine-N-[maleimide(polyethylene glycol)-2000](ammonium salt) in varying percentages (0.15%/0.3%/0.6%/0.9%/1.2%) as replacement for DMG-PEG 2000 (total lipid PEG ratio did not exceed 1.5%). In this study, the peptides under investigation were designed with dual objectives: firstly, to target the extracellular matrix of vascular smooth muscle cells (VSMCs) with a peptide targeting collagen 4 (Col-4; KLWVLPK-GGG-C(SEQ ID NO:48)), thereby enhancing the retention of LNPs around these cells; and secondly, to target surface receptors expressed on VSMCs to facilitate the internalization of LNPs. The sequences of the peptides targeting surface receptors were TL6R; C-GGG-LSLITRL (SEQ ID NO:49), Gal-3; C-GGG-ANTPCGPYTHDCPVKR (SEQ ID NO:51), and CD63; CRHSQMTVTSRL-GGG (SEQ ID NO:50).

For conjugating peptides to LNPs, the peptides were initially treated with tris(2-carboxyethyl)phosphine (TCEP) reducing agent (Thermo Scientific, 77712). Subsequently, the free peptides were incubated with Mal-functionalized LNPs for 1 hour at pH 7.4, with a peptide to Mal molar ratio of 2:1. The final product of peptide-conjugated LNPs was dialyzed against phosphate buffered saline (PBS) and concentrated using Amicon® tubes. To assess the efficiency of conjugation, a maleimide fluorometric detection kit (Sigma, MAK167) was utilized following the protocol of the manufacturer.

### LNPs Characterization

The size (diameter) and surface charge (zeta potential) of the LNPs were determined via dynamic light scattering (DLS) using the Zetasizer® Nano ZS (633 nm, Malvern Instruments), with light collection at a scattering angle of 173°. Nucleic acid encapsulation efficiency was assessed employing the modified Quant-it™ RiboGreen® RNA assay (Invitrogen™). Initially, a nucleic acid standard curve was prepared in TE (Tris-EDTA) buffer. LNPs were subsequently diluted either in TE buffer or TE-Triton (TE supplemented with 2% Triton-X100) to match the nucleic acid concentration within the standard curve. Duplicates of LNPs and standard curve samples (100 μL each) were loaded into a black 96-well plate, followed by the addition of 100 μL of RiboGreen® reagent (diluted 1:200 in TE buffer). Fluorescence intensity originating from unencapsulated nucleic acid (I_{frec}) was measured using a plate reader (Excitation: 485 nm, Emission: 528 nm, Infinite® M plex, TECAN®). Triton-treated LNP samples represented released nucleic acid from disrupted LNPs, while intact intensity of LNPs indicated unencapsulated nucleic acid. Encapsulation efficiency was computed as % EE=100×(1−(I_{intact}/I_{disrupted})).

### Perfusion of Human Vessels

The perfusion bath for tubular organs (PBTO) system, peristaltic pump, and water bath containing sterile water at 37° C. were assembled inside a fume hood disinfected with 70% ethanol. The entire plumbing system was sterilized using 2% mucasol, followed by perfusion with 500 mL of physiological PBS for 1 hour to ensure sterility. Blood vessels were collected from the operating room (OR) in DMEM supplemented with 10% FBS and transferred under sterile conditions. Upon receipt, a ˜6 cm vessel segment was cannulated into the PBTO system using a suture kit. The vessel was initially perfused with 10% FBS/DMEM for 15 minutes to assess potential leaks, which were sutured if detected. Next, 500 μL of AAVPR-GFP (4.98×10¹¹ vg) or AAV9 (5.3×10¹¹ vg) was injected into the vessel lumen and perfused through the system at a flow rate of 22 mL/min for 1 hour. Following perfusion, the vessel was decannulated and maintained in a CO₂ incubator for 7 days. At the end of the culture period, AAV-perfused vessels were sectioned for downstream analyses, with 5 mm segments allocated for immunofluorescence, 5 mm for tissue clearing and immunofluorescence, and 3 mm for PCR. See, e.g., Bianco et al., A Protocol for a Novel HumanEx VivoModel of Aneurysm, STRAR ptotocol, 1, 100108. doi.org/10.1016/j.xpro.2020.100108; Lipskaia er al., (2013) SERCA2a gene transfer prevents intimal proliferation in anorgan culture of human internal mammary artery. Gene Ther. 20(4): 396-406. doi:10.1038/gt.2012.50.

### Histology of Human Vessels

Tissue sections were washed in PBS three times for 5 minutes each at room temperature to remove residual media and debris. Samples were then permeabilized using 0.3% PBTx for 2 hours at room temperature, followed by blocking in a solution containing 0.3% PBTx, 2.5% normal donkey serum (NDS), and 1% bovine serum albumin (BSA) for 45 minutes at room temperature to minimize nonspecific antibody binding. Unconjugated GFP primary antibody (Abcam) was diluted to 1:150 in blocking buffer, and slides were incubated with the primary antibody overnight at 4° C. The next day, slides were washed in PBS three times for 5 minutes each at room temperature before incubation with αSMA-Cy3 and a secondary antibody (Rb 647) diluted to 1:500 in blocking buffer for 1 hour at room temperature. Following secondary antibody incubation, slides were washed again in PBS three times for 5 minutes each and mounted using DAPI-containing mounting media. Images were acquired using an LSM Airyscan 800 confocal microscope.

### Example 1. Constructs for Delivering Soluble Secreted Recombinant ENPP1

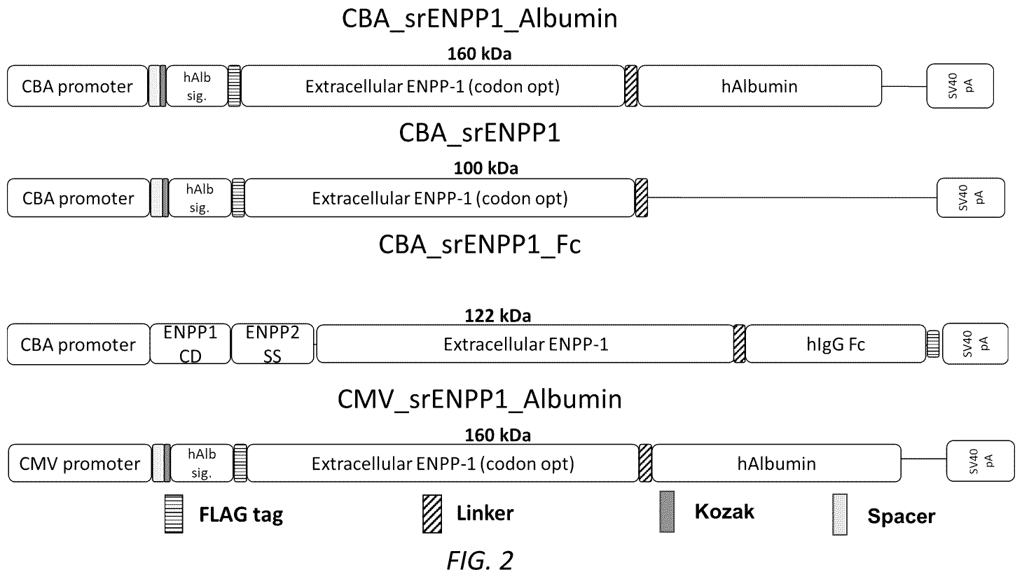

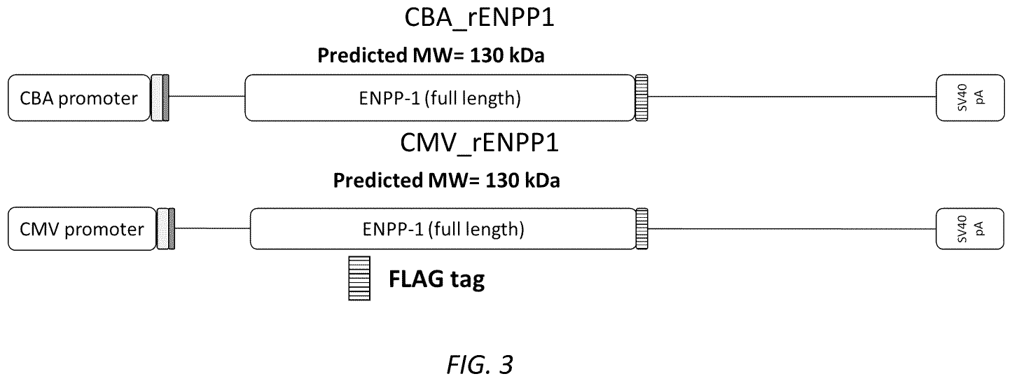

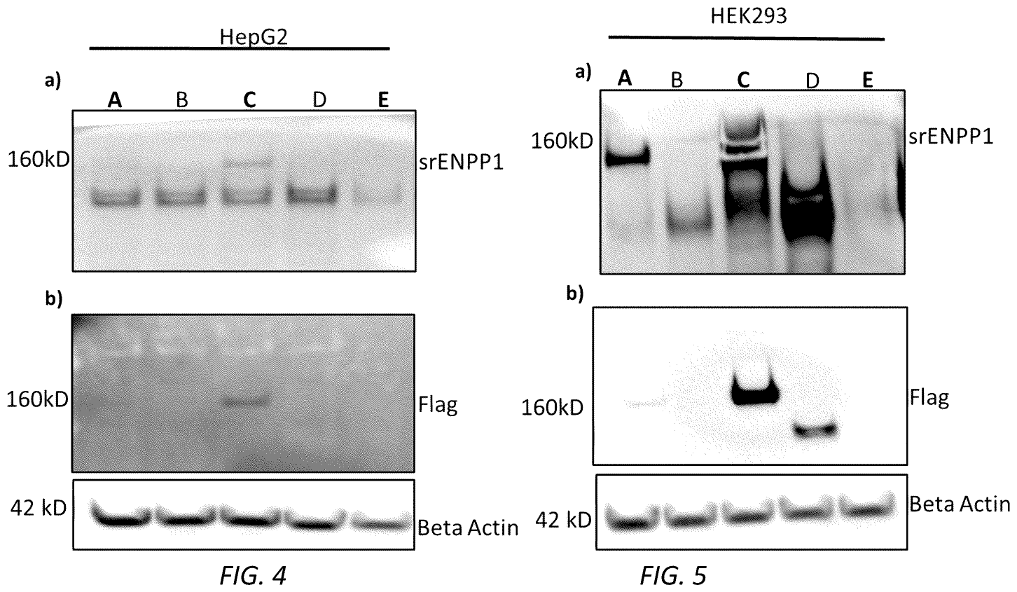

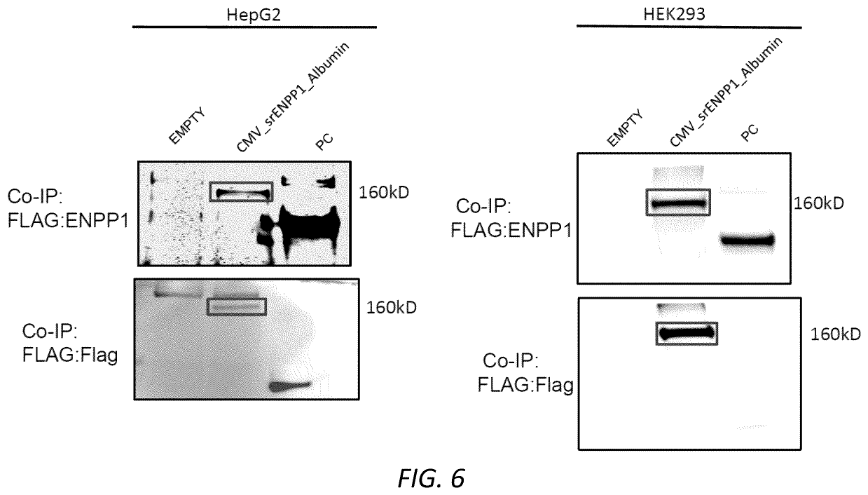

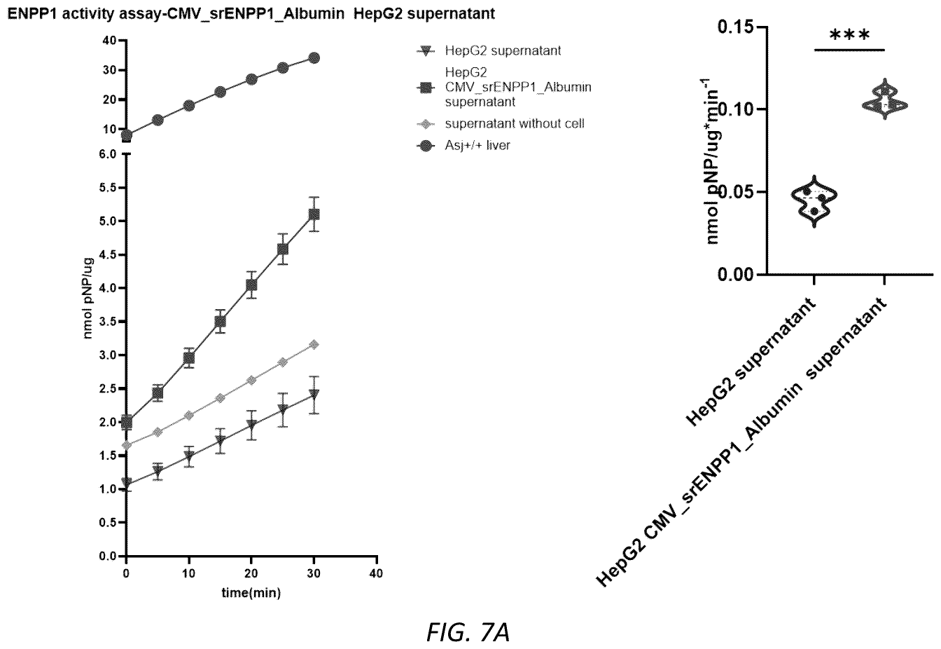

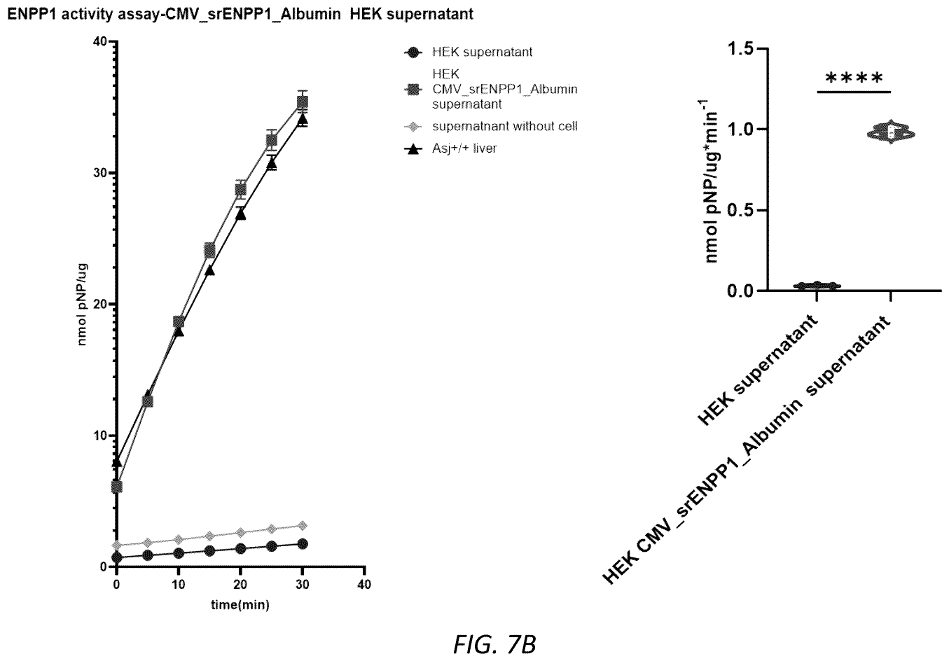

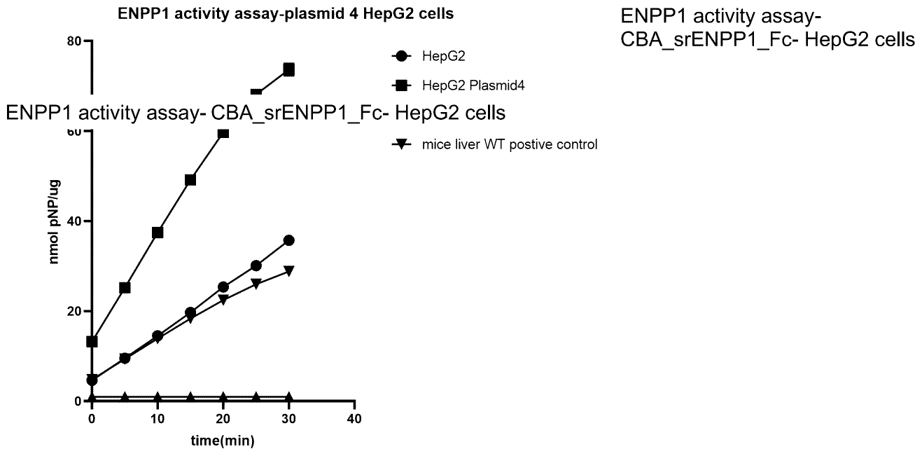

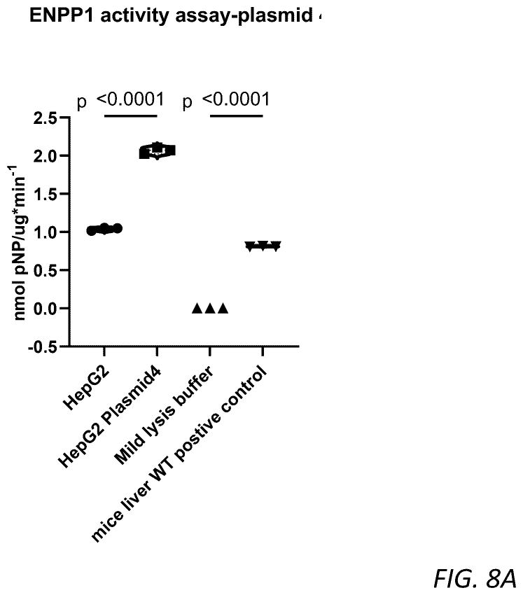

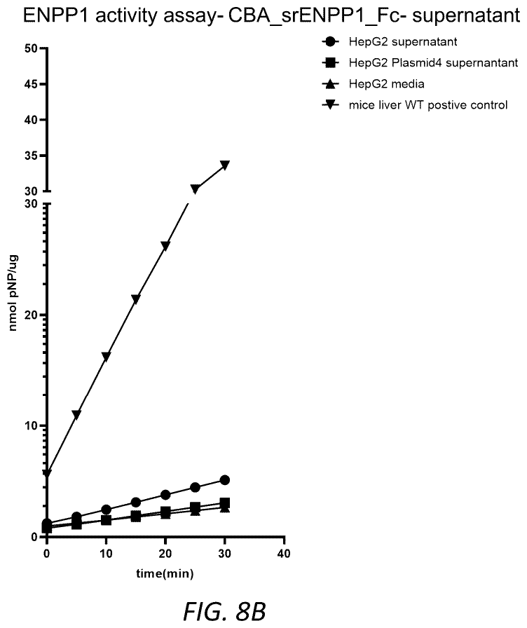

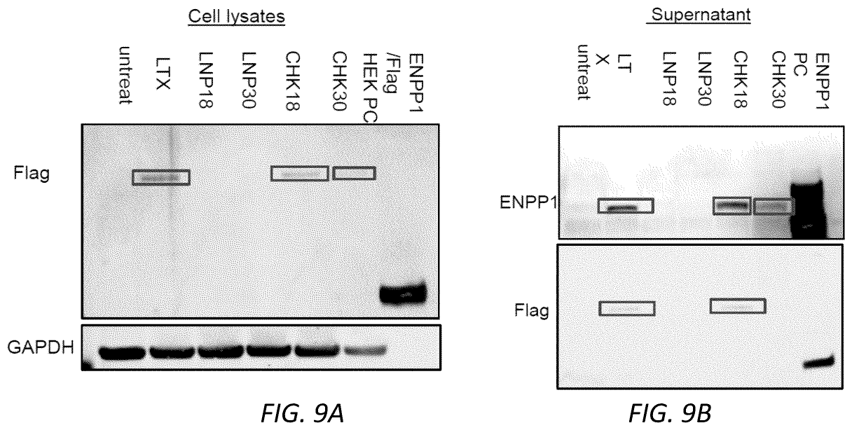

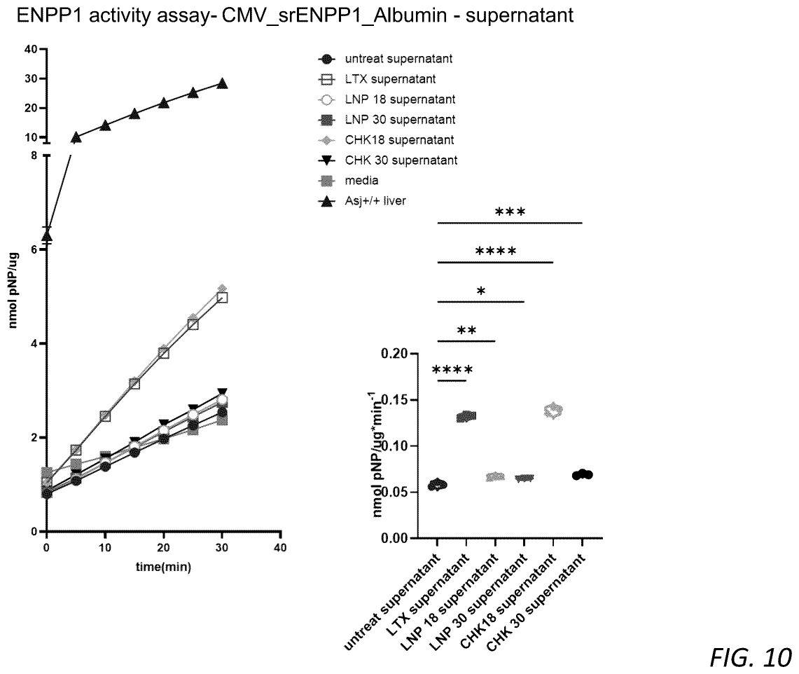

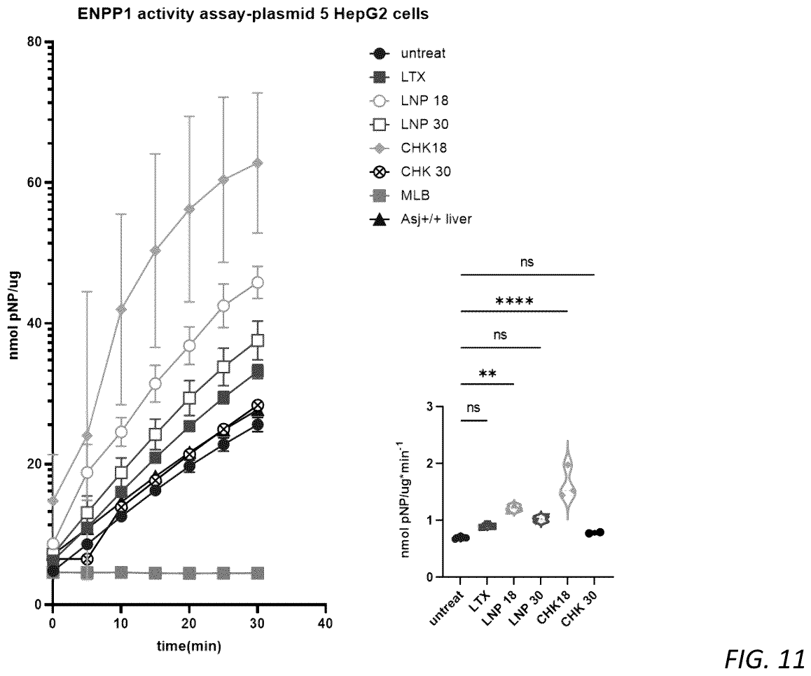

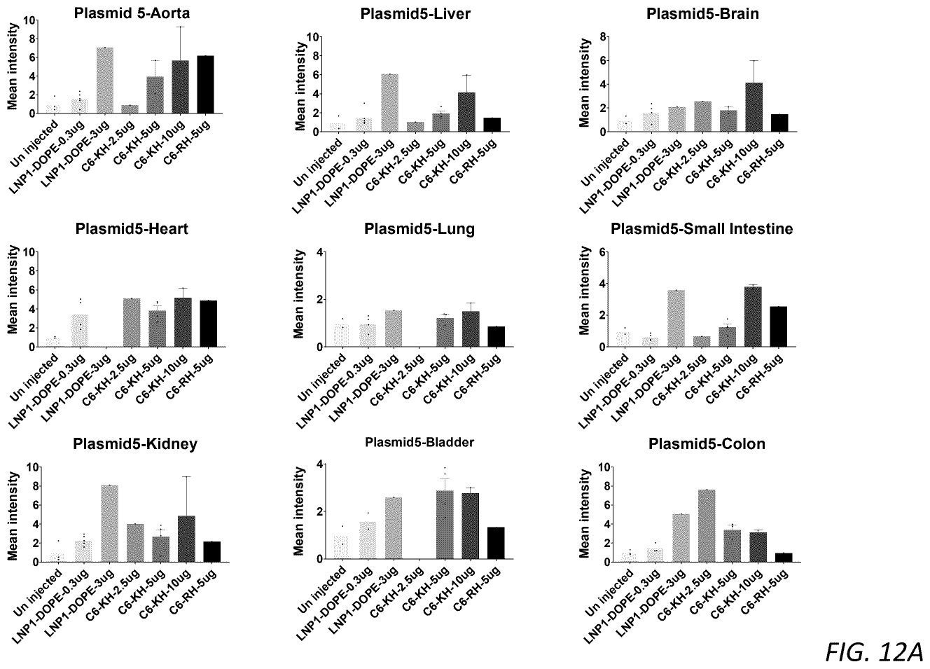

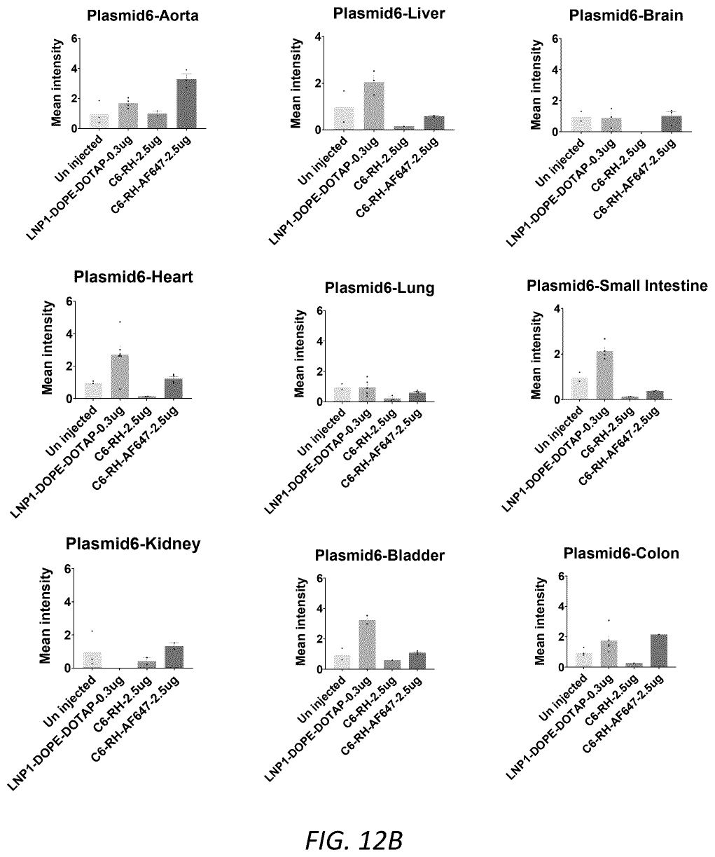

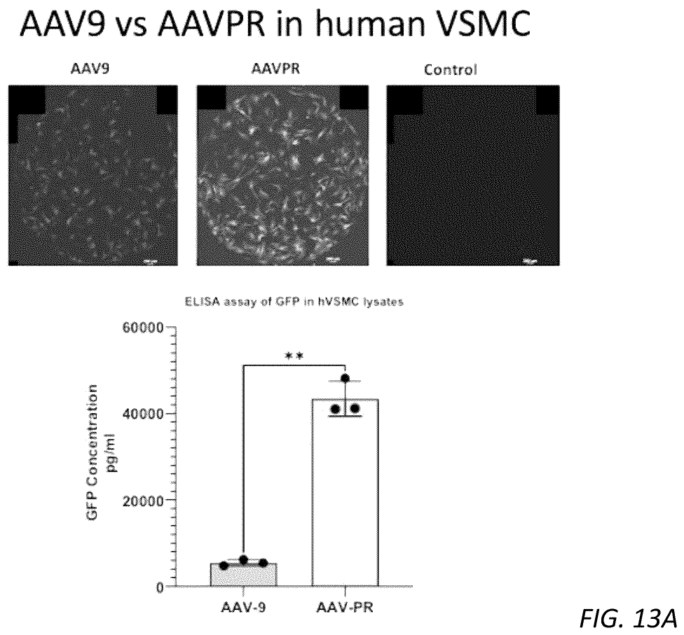

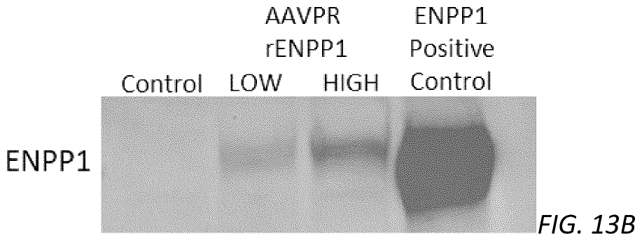

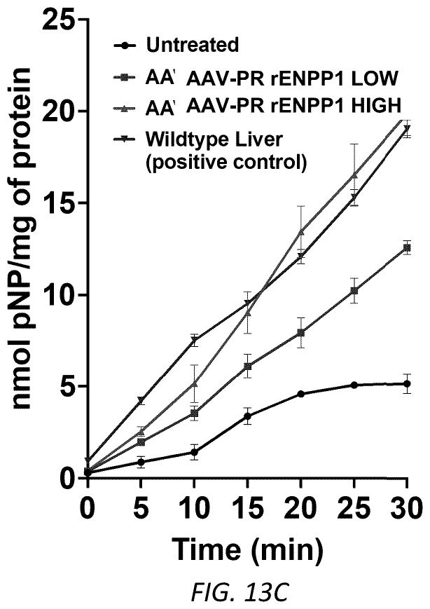

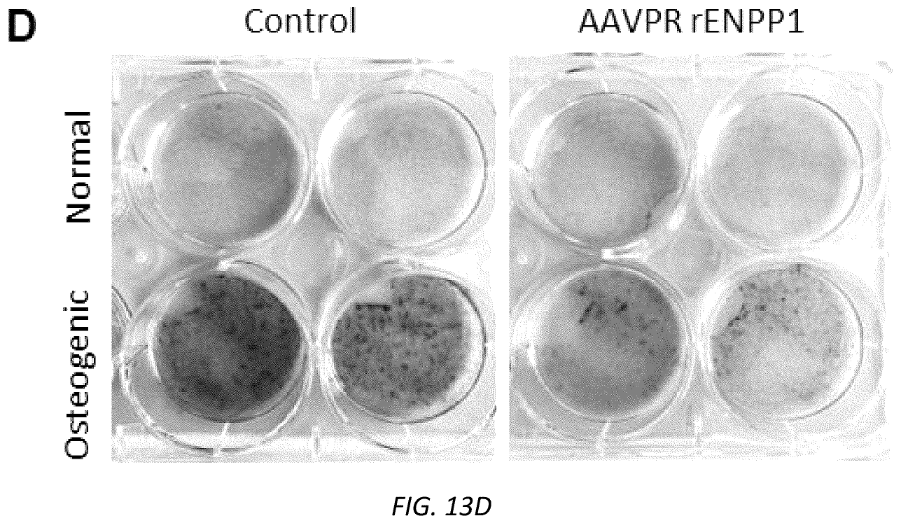

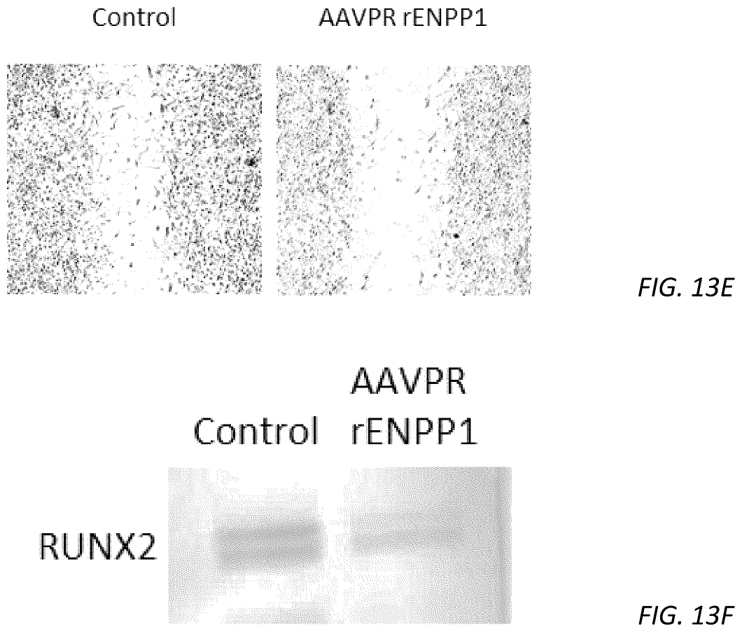

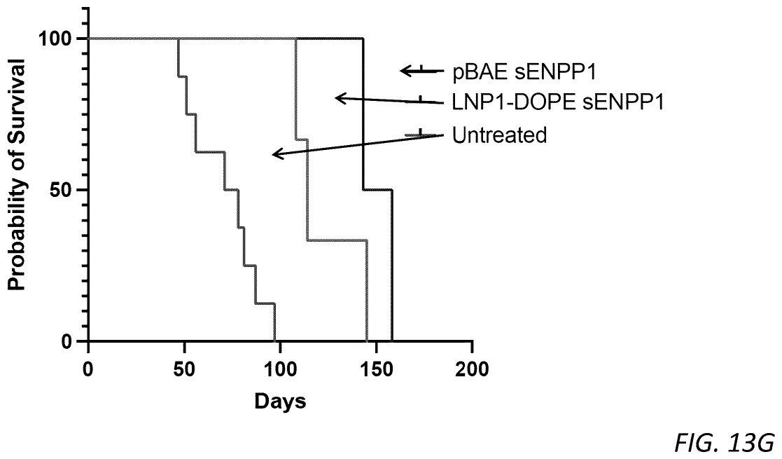

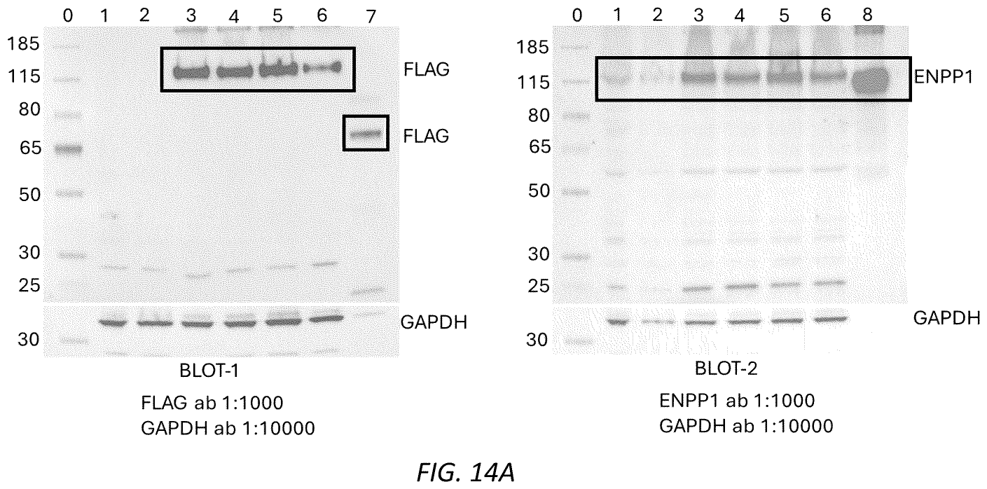

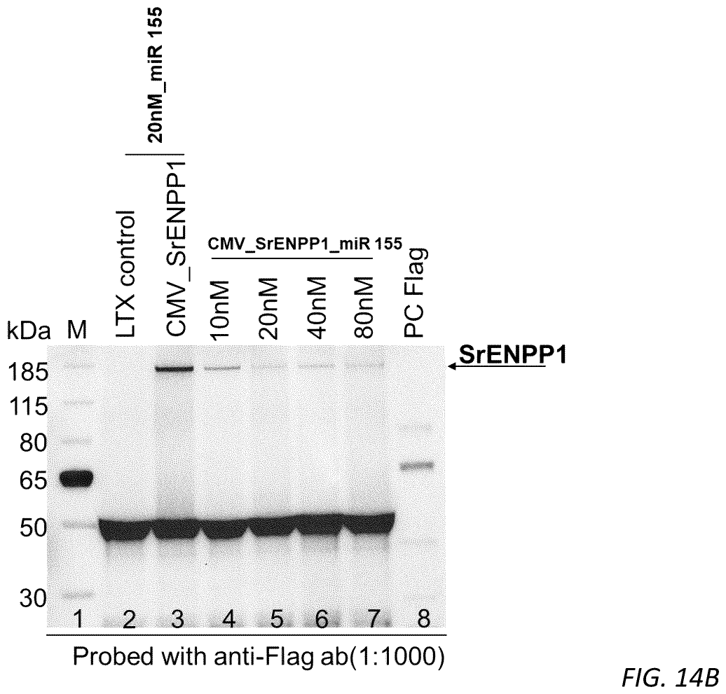

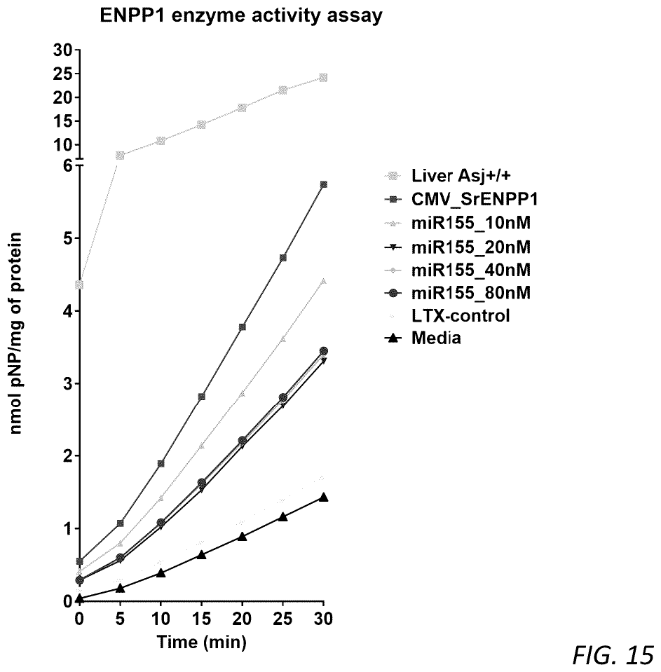

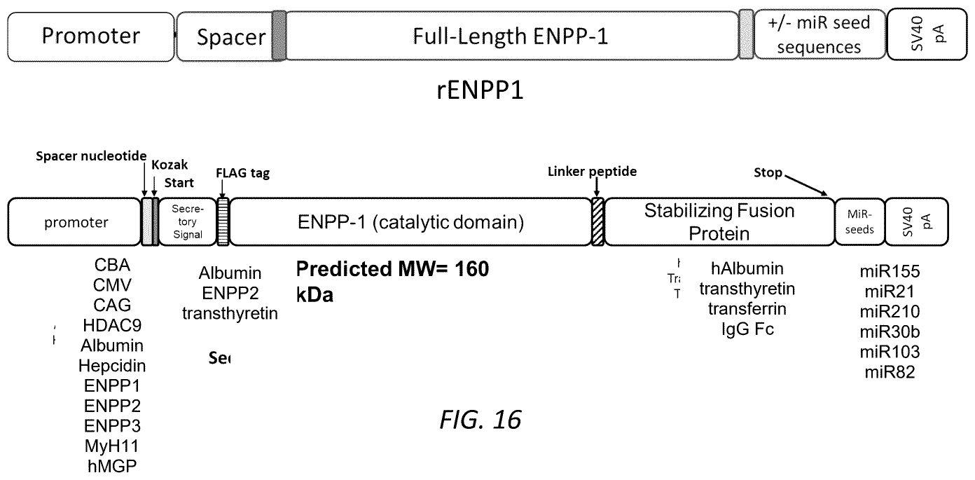

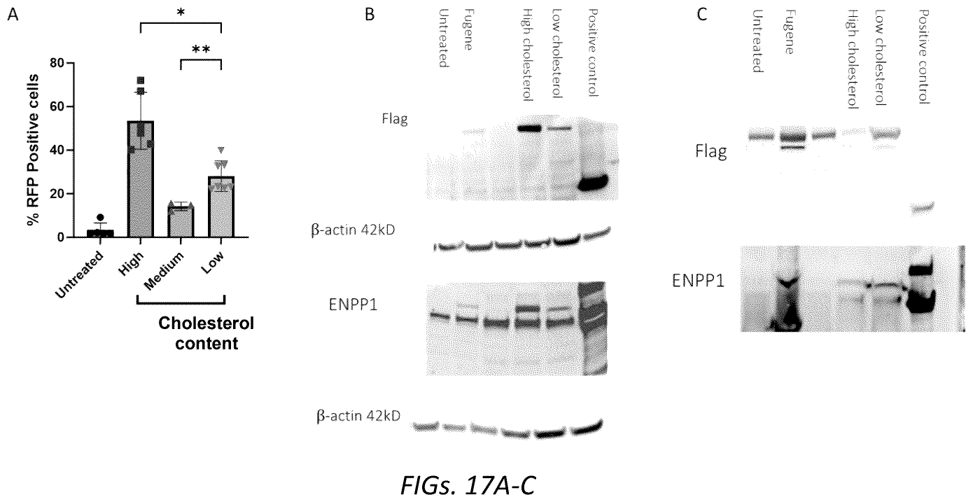

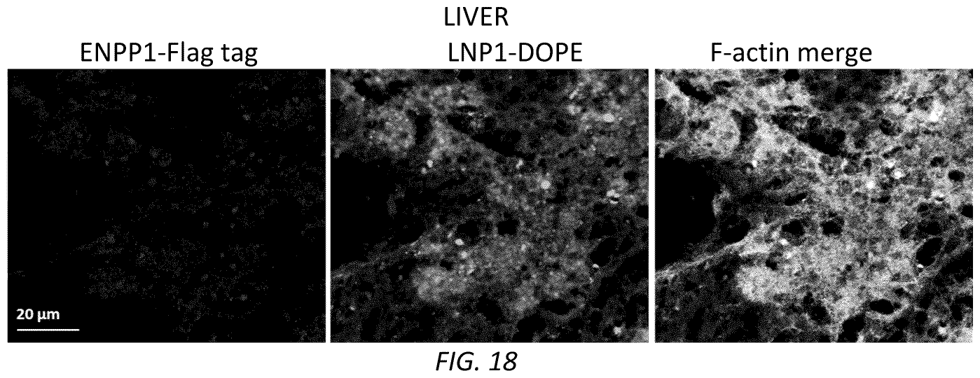

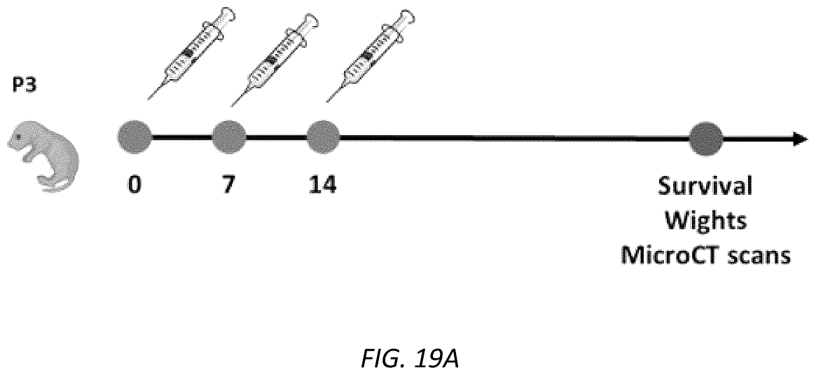

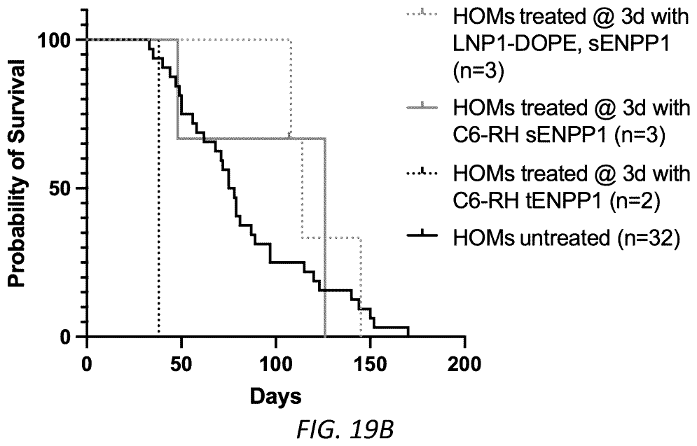

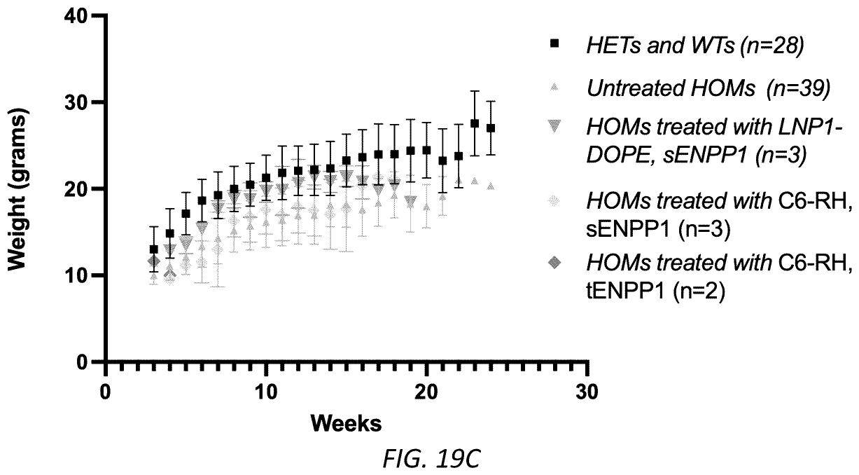

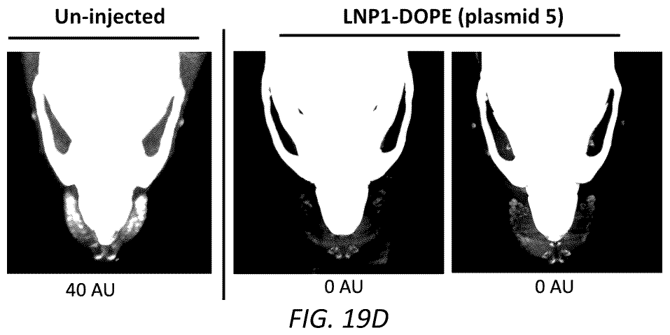

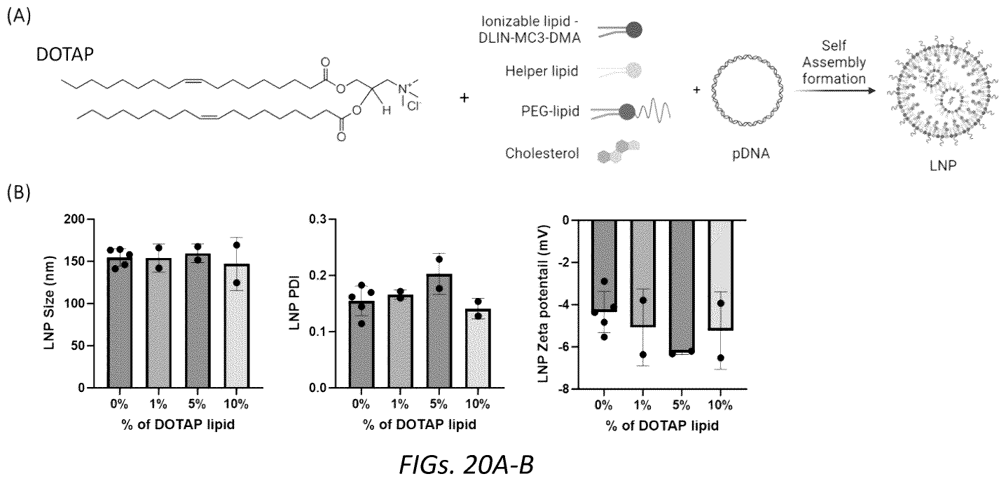

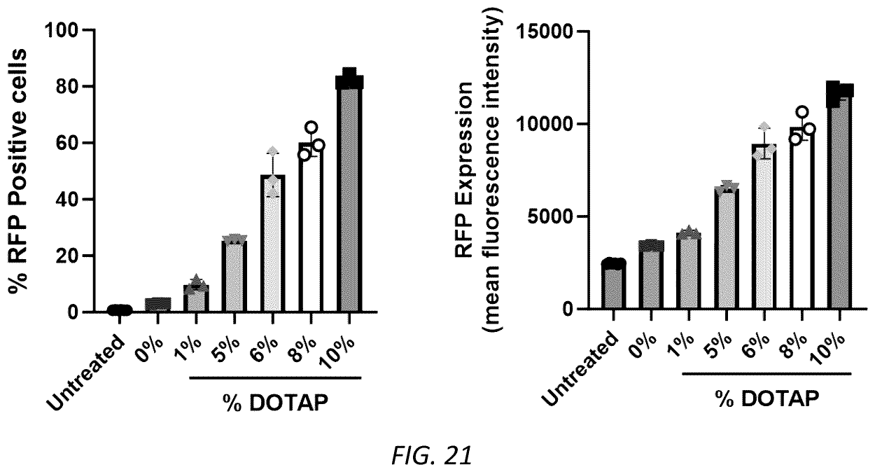

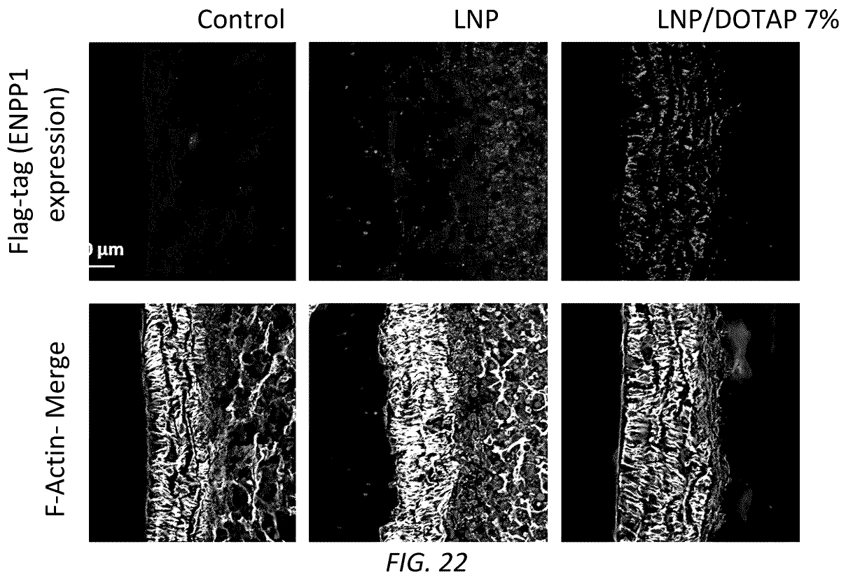

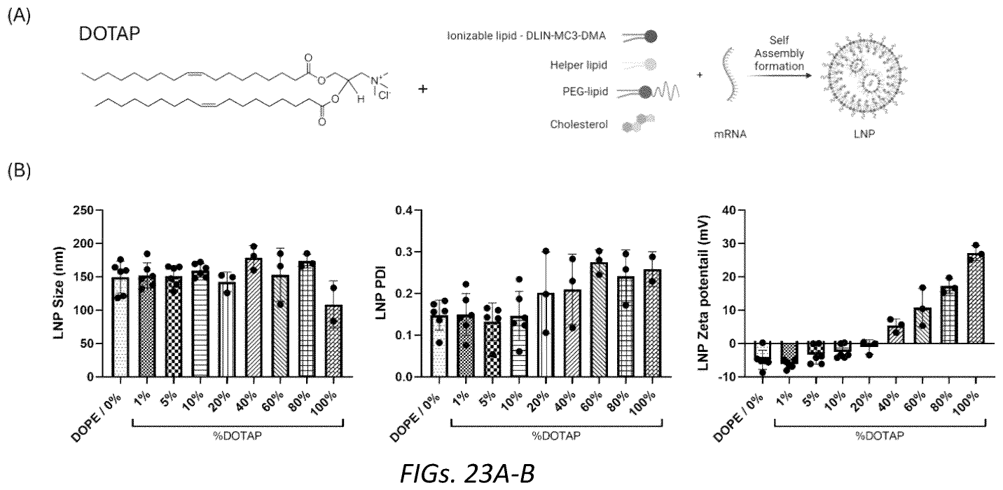

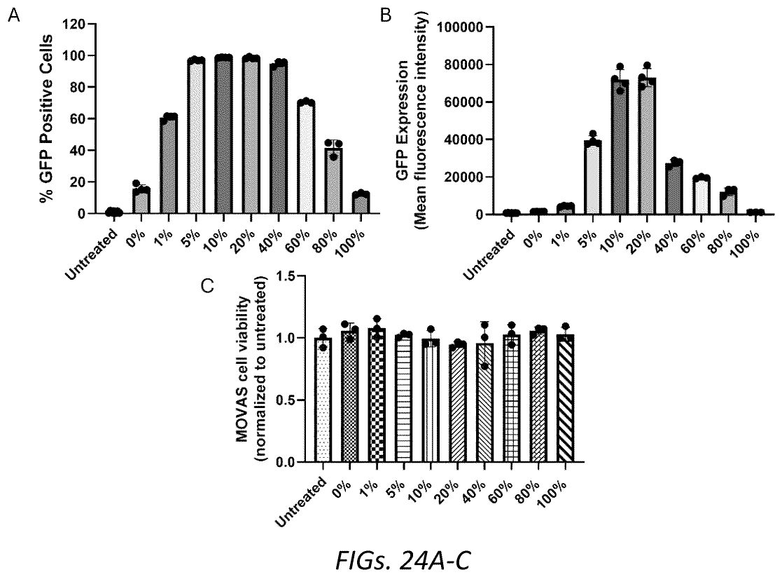

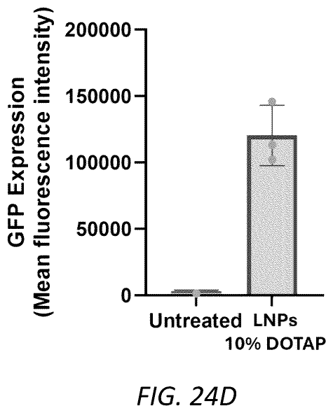

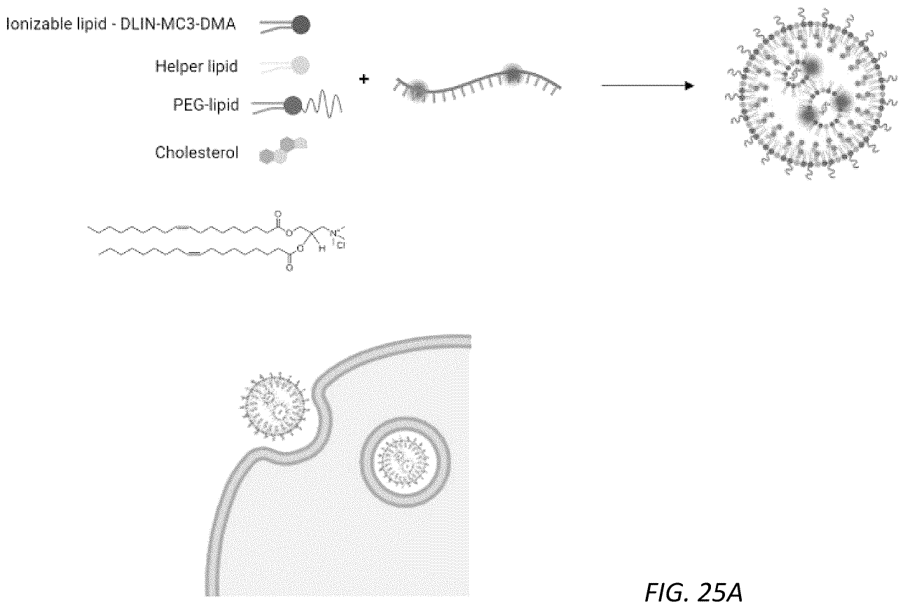

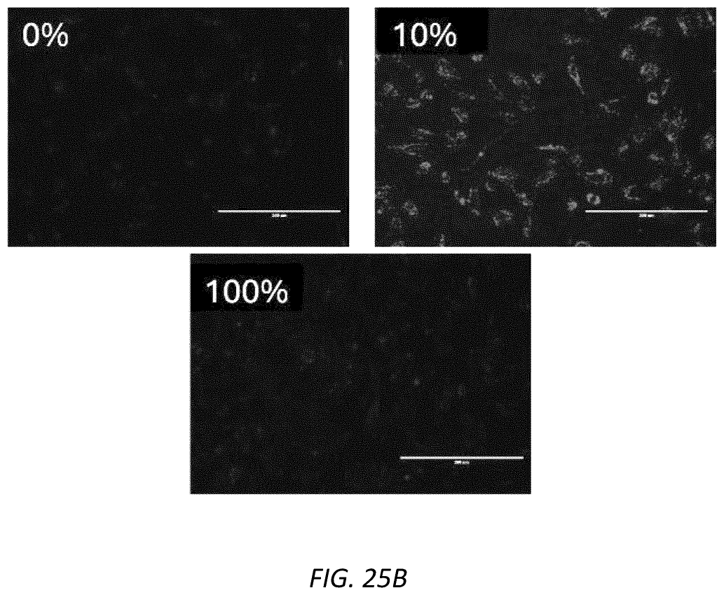

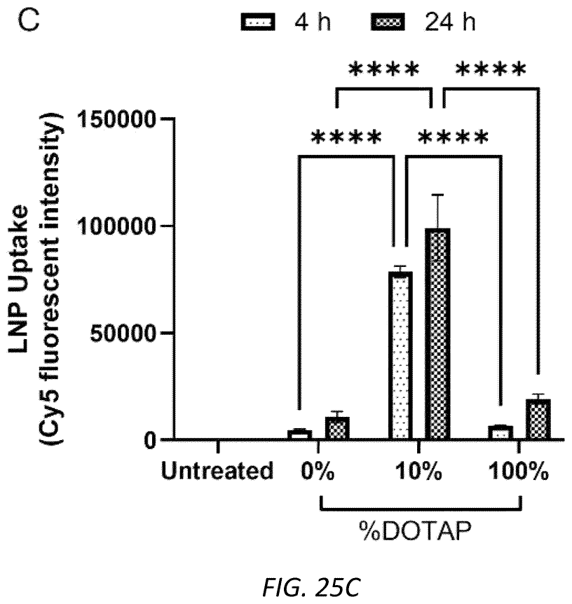

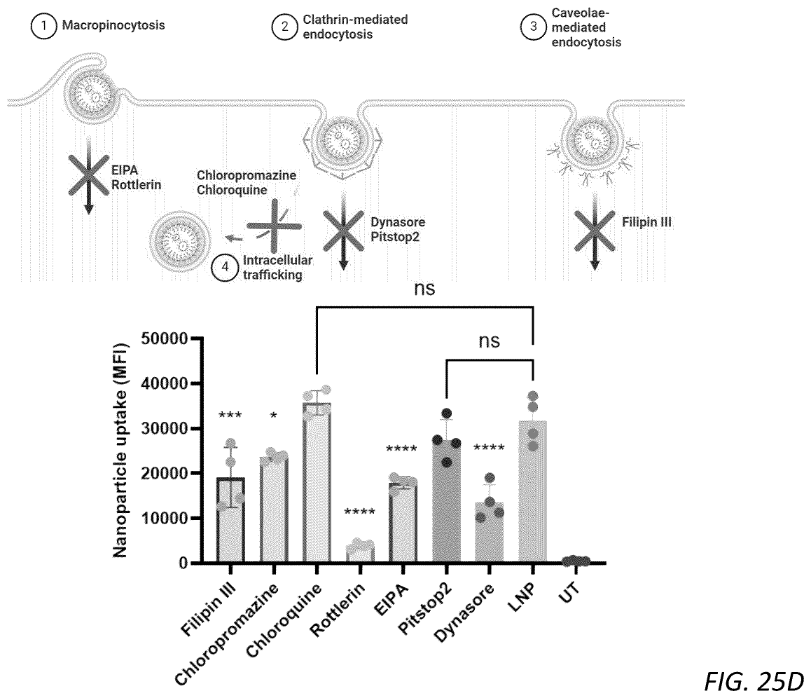

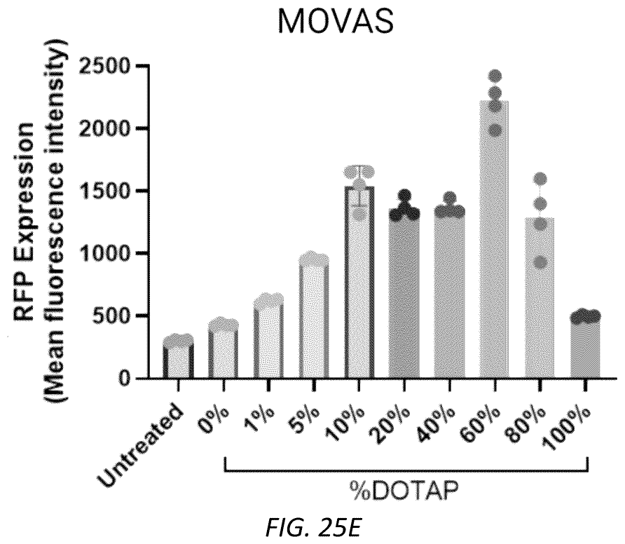

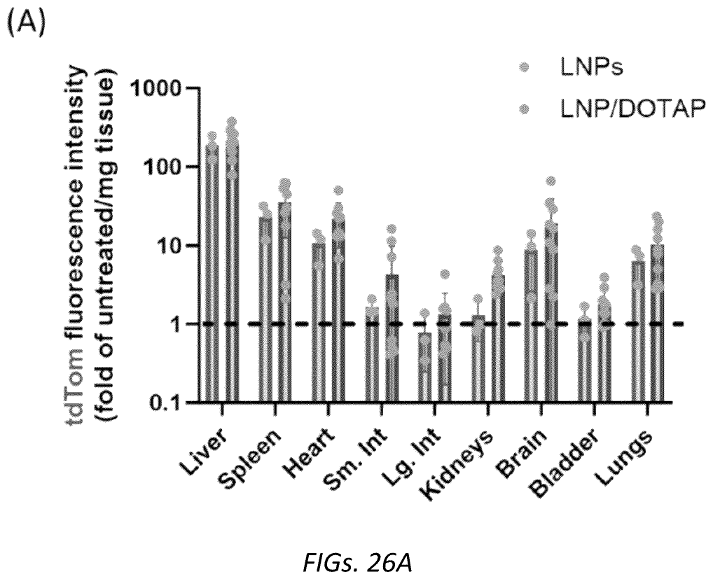

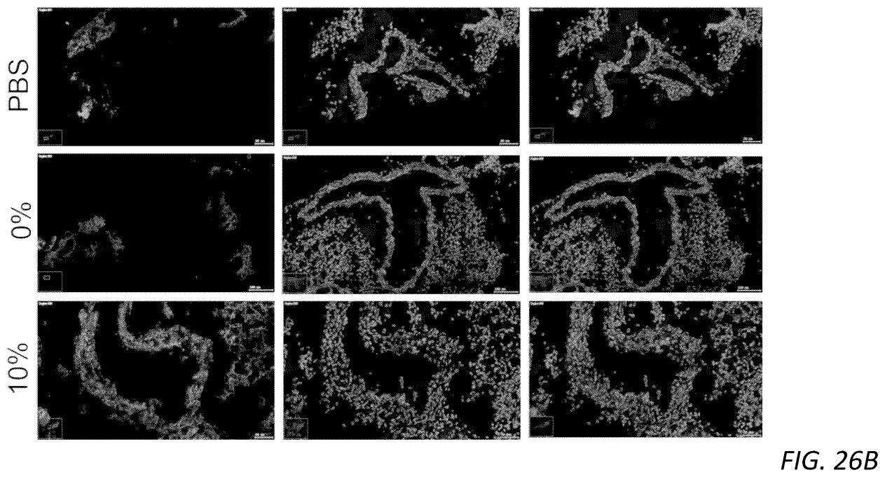

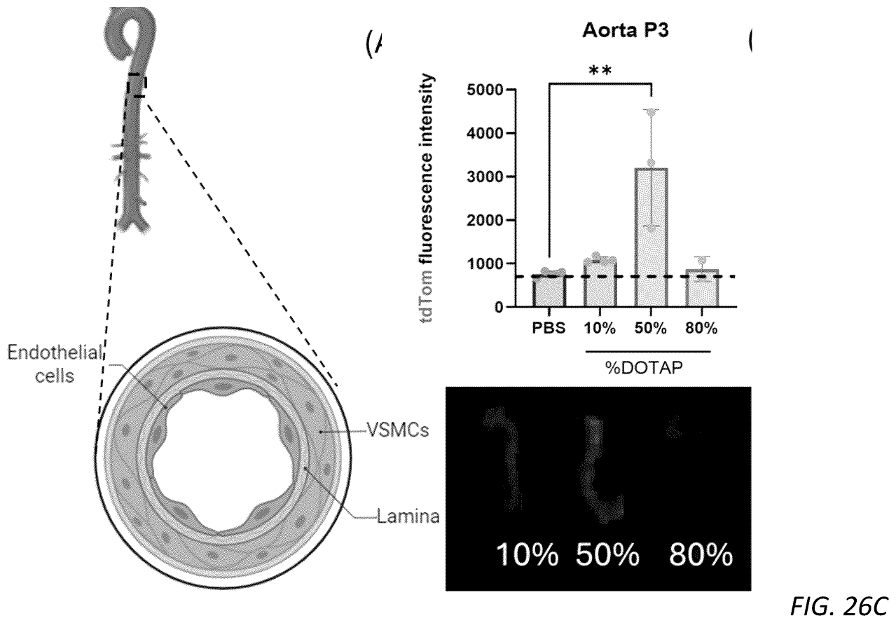

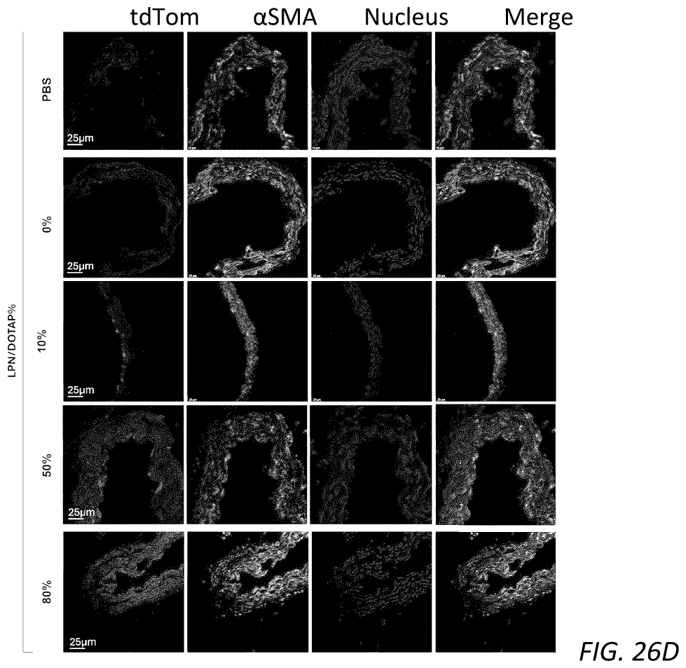

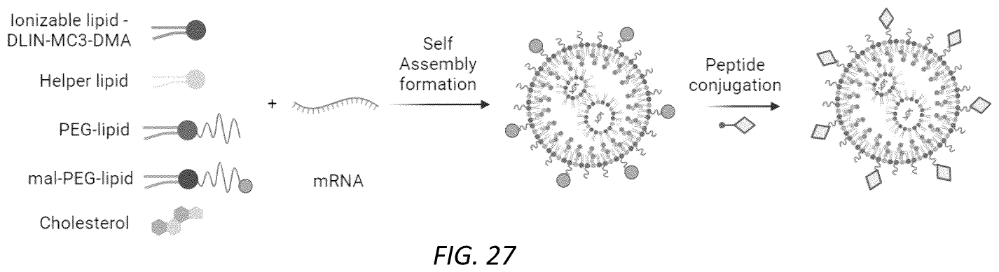

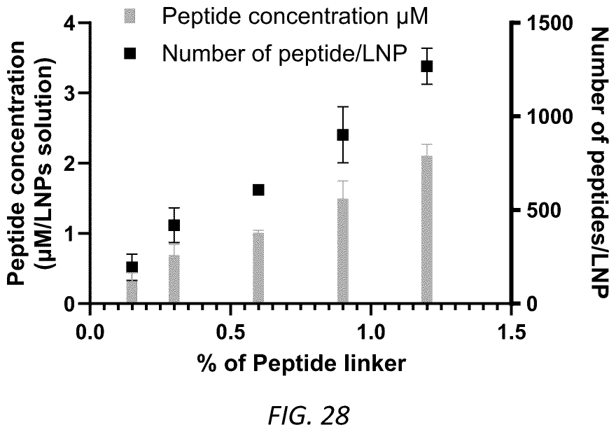

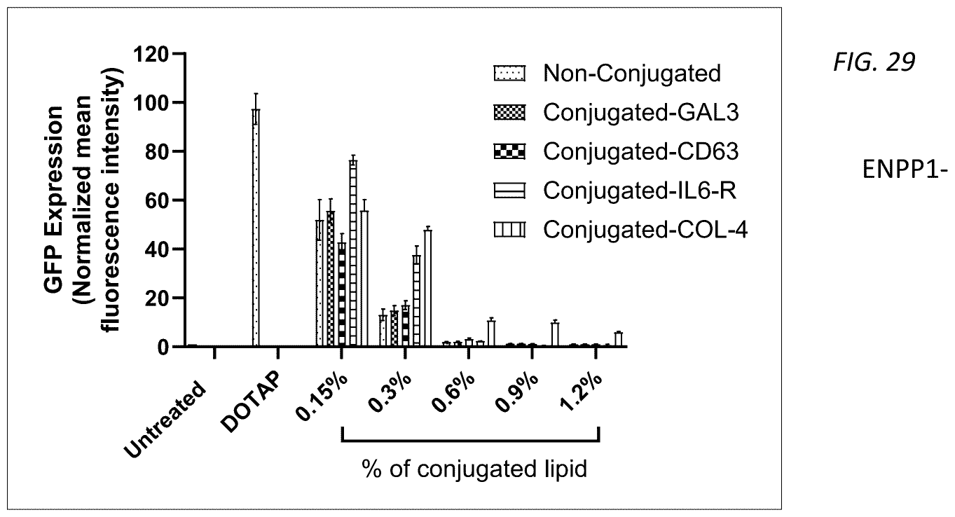

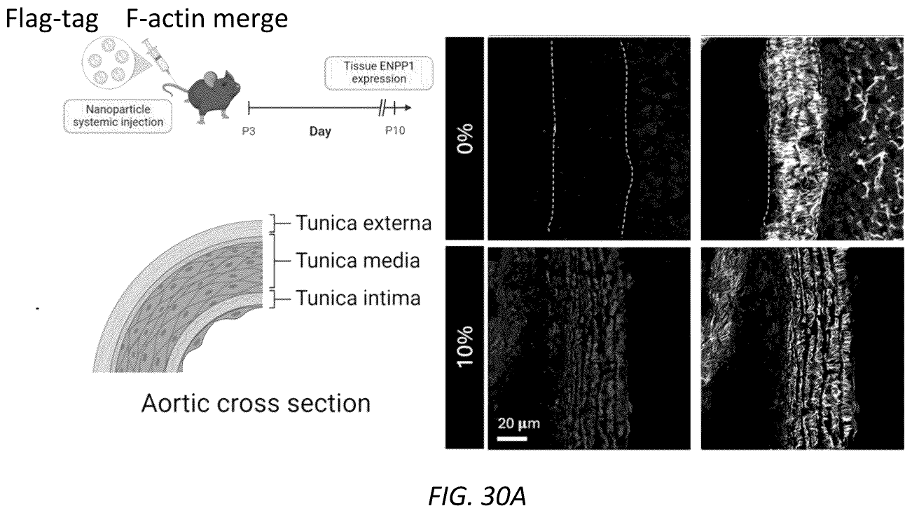

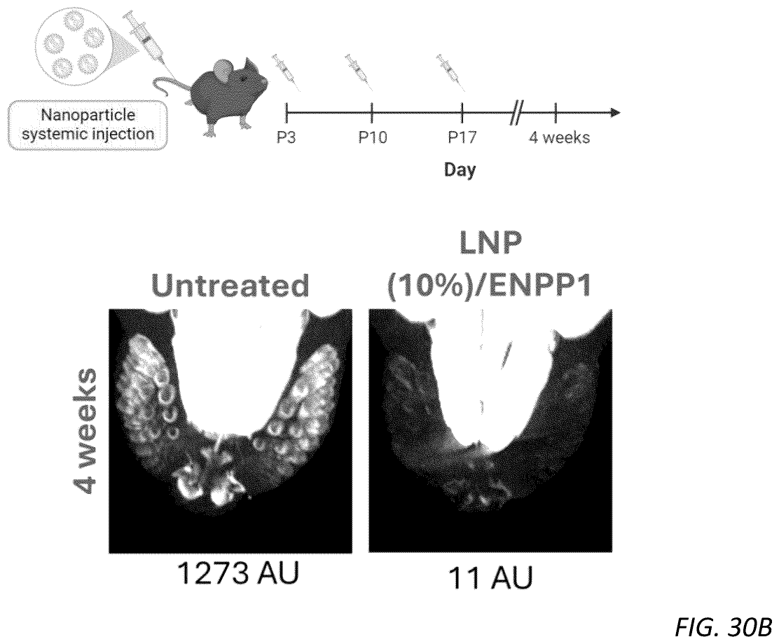

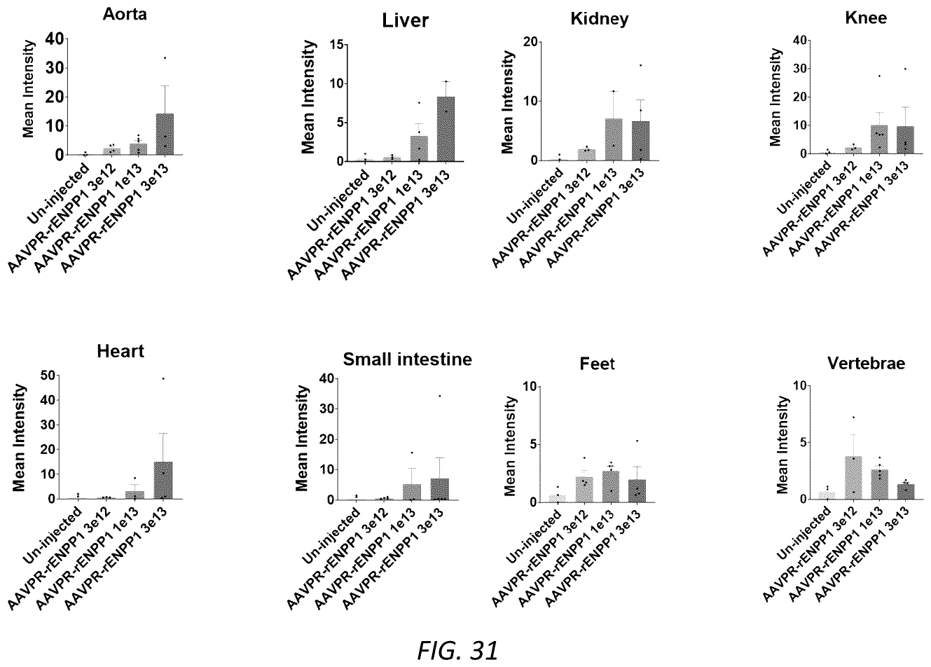

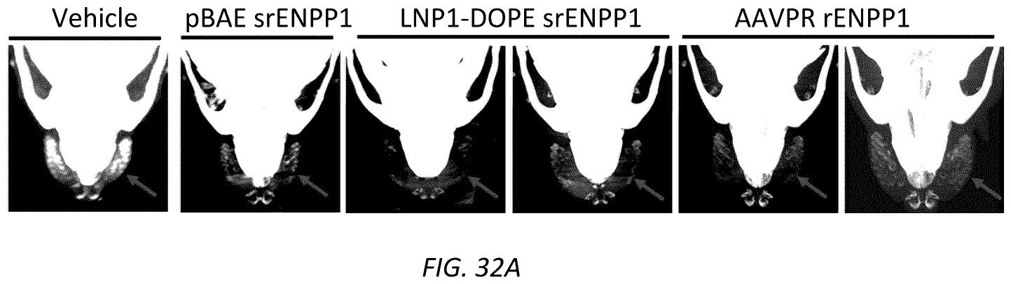

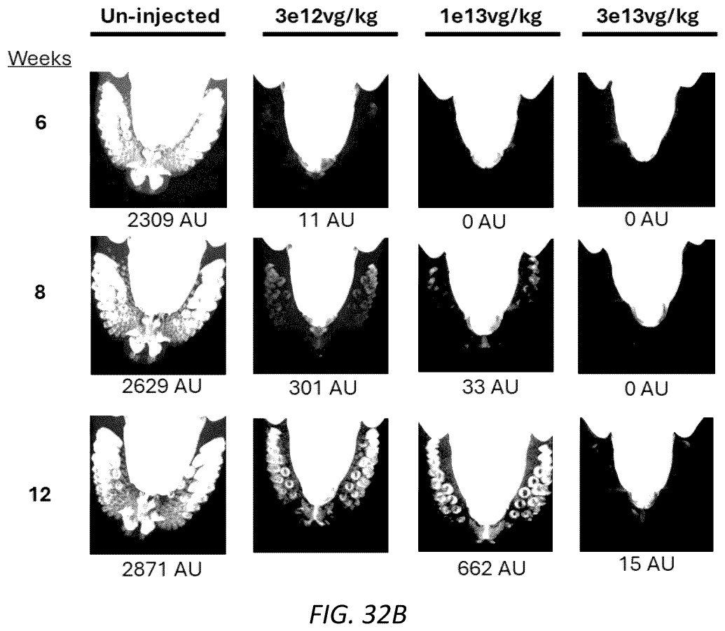

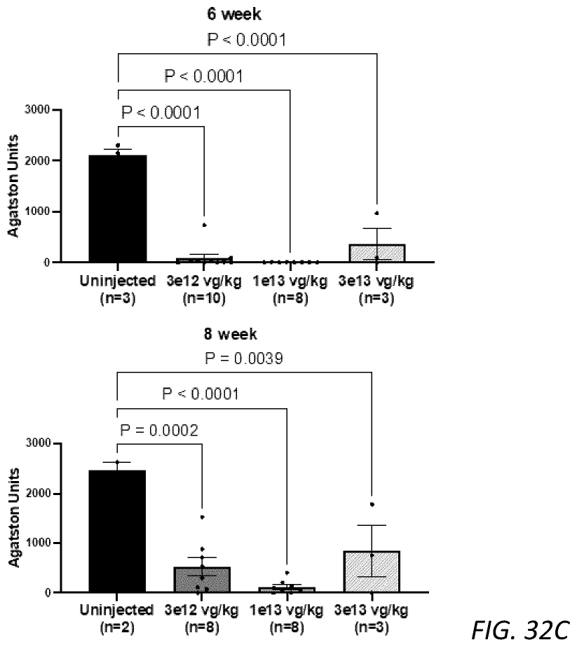

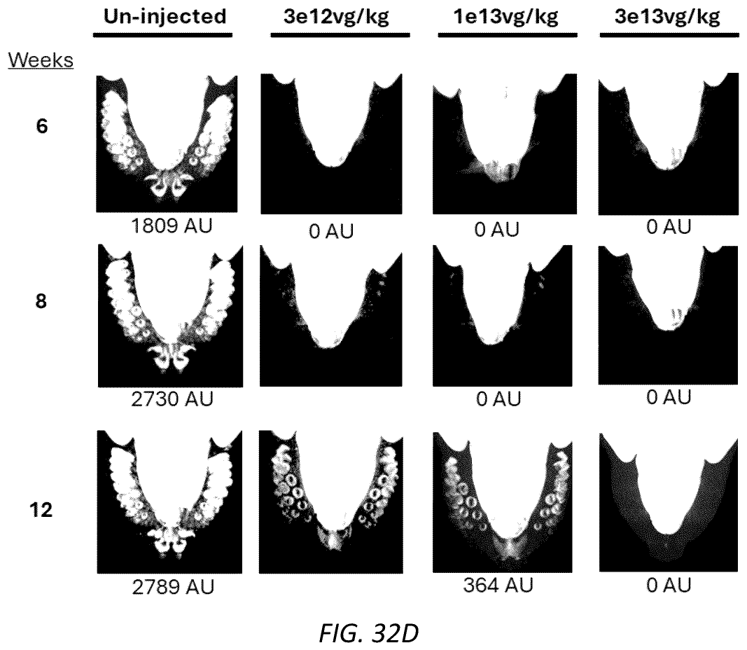

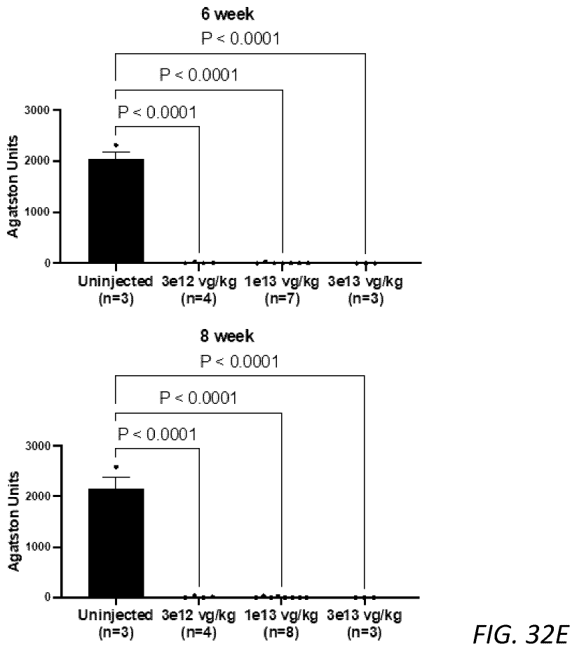

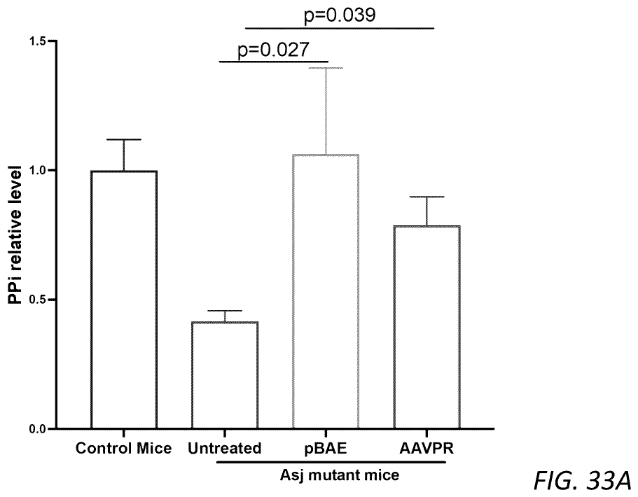

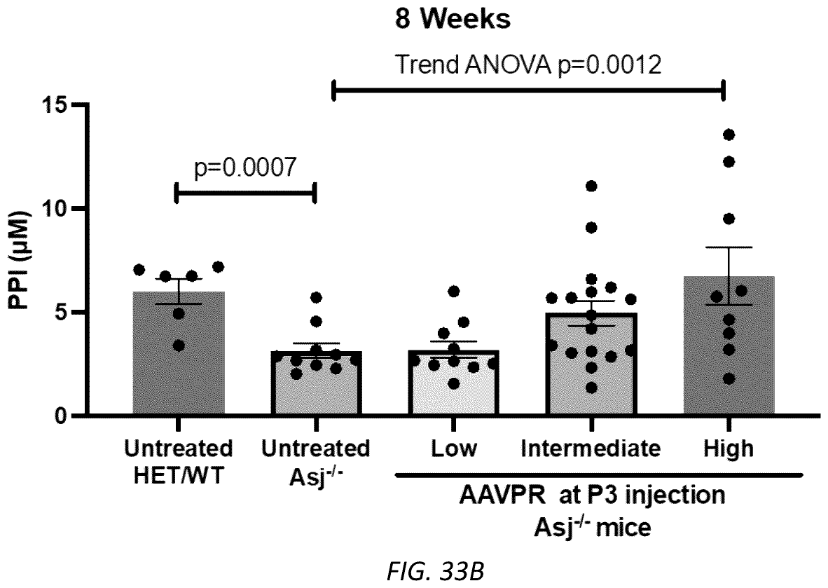

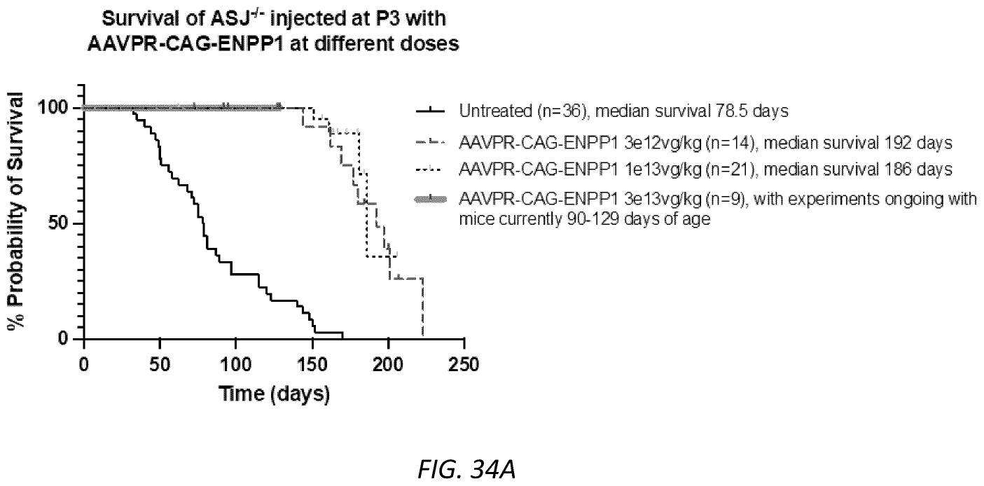

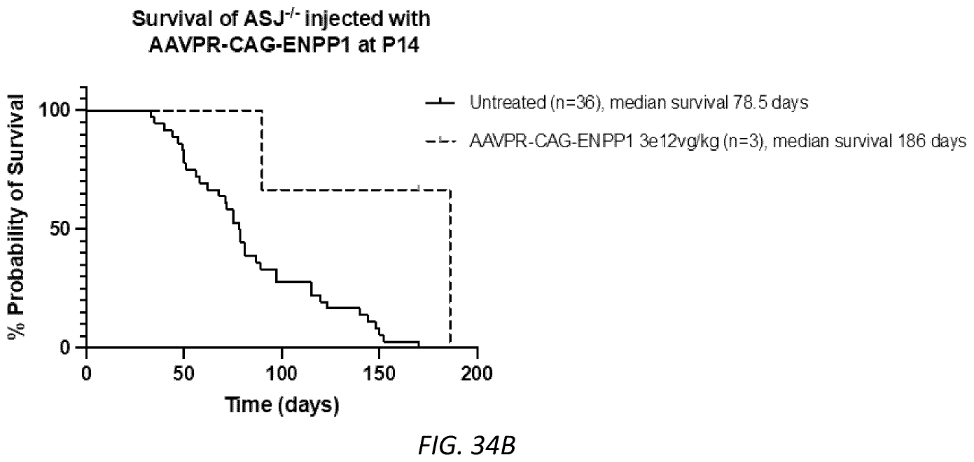

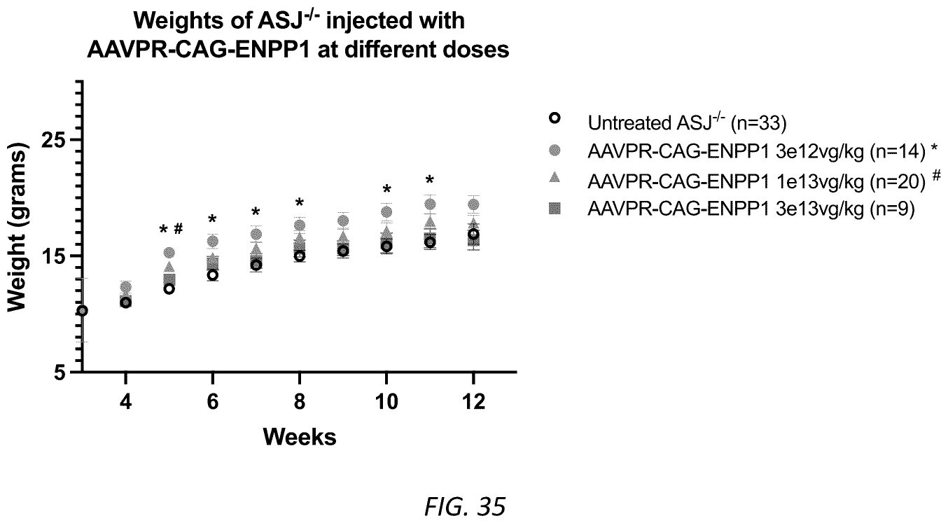

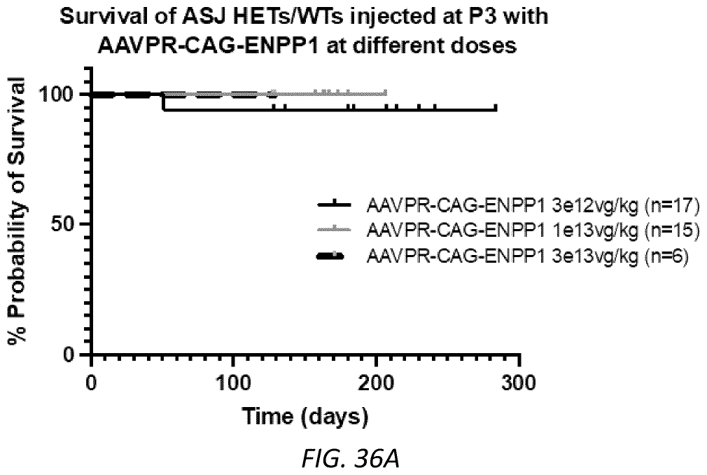

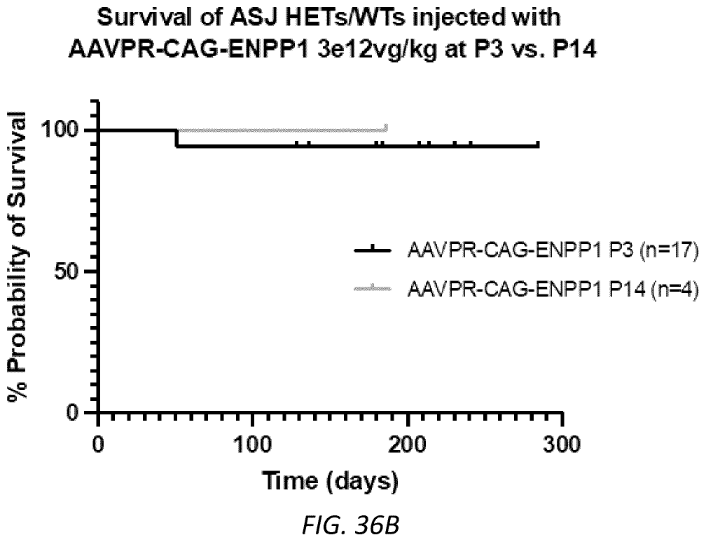

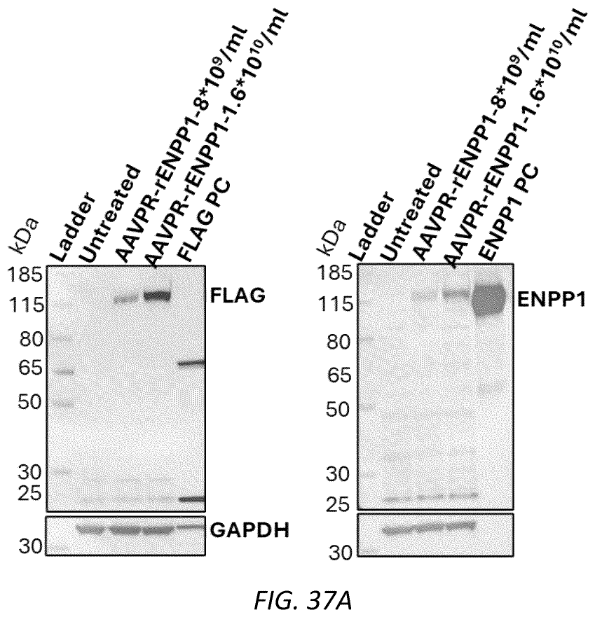

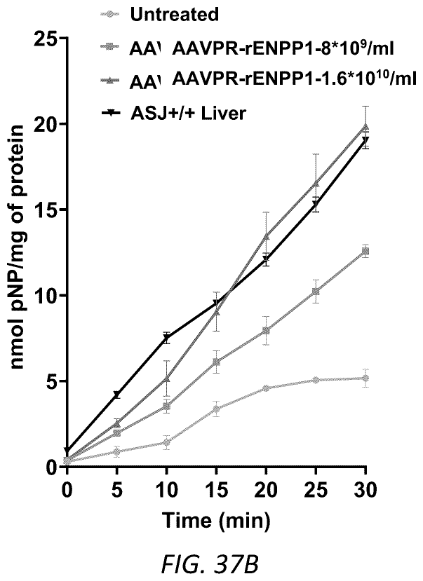

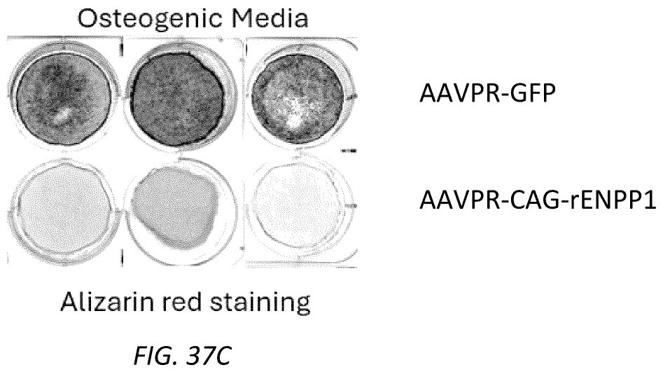

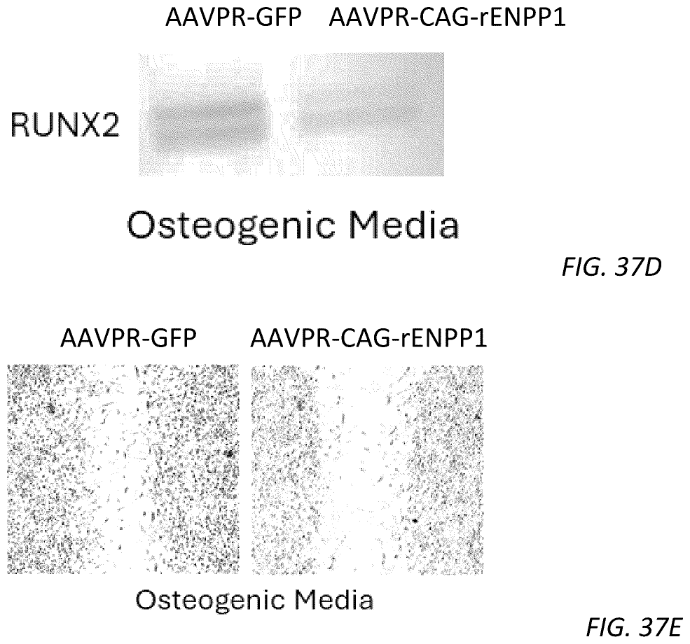

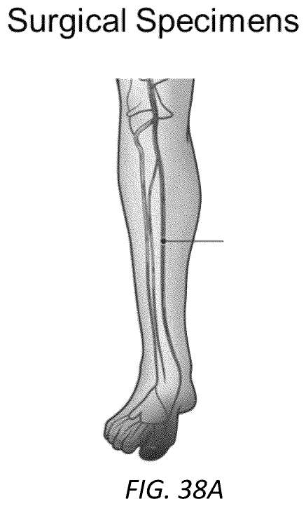

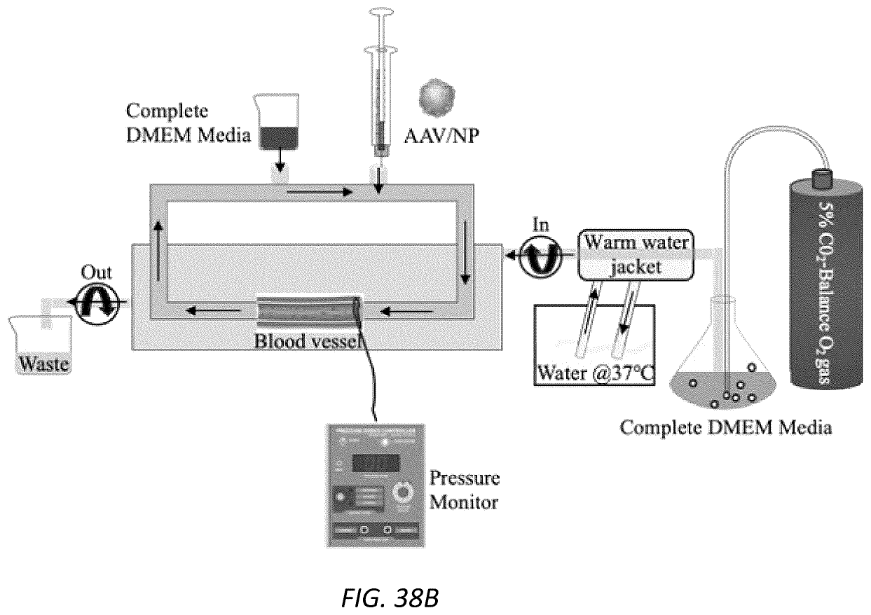

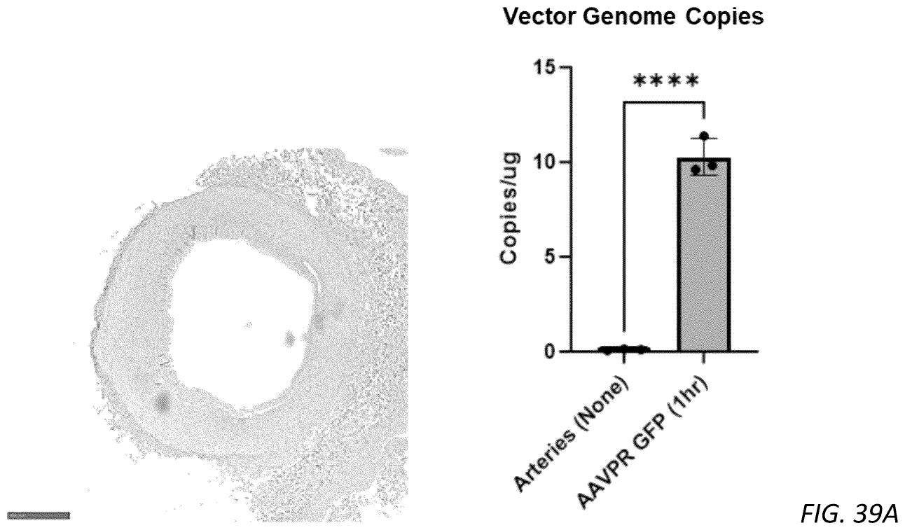

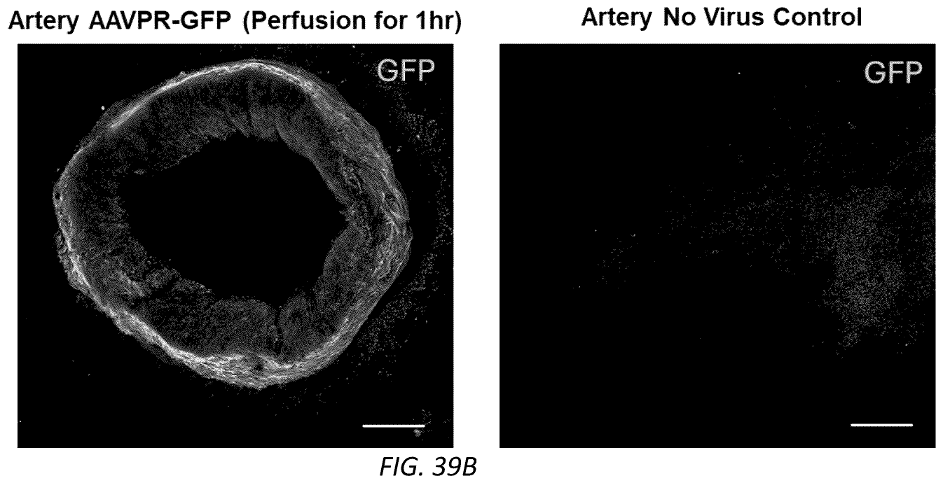

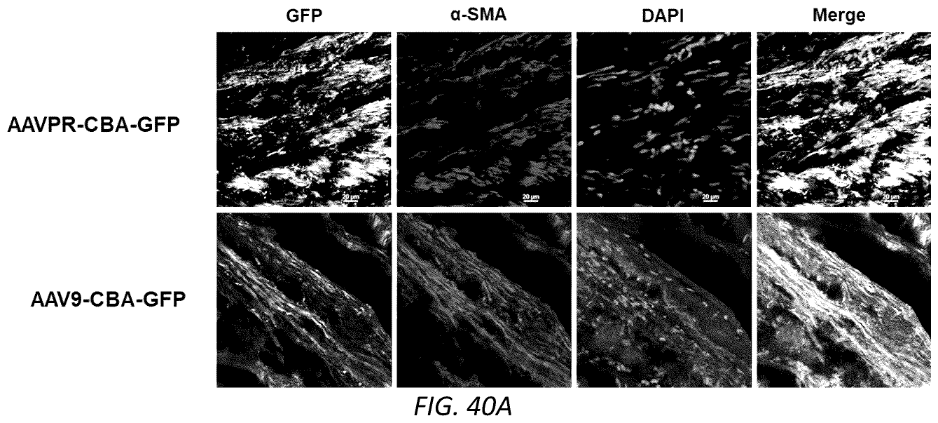

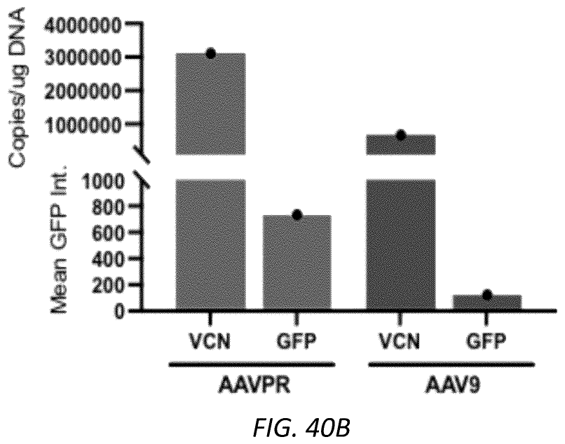

DNA constructs were designed with the aim of delivering soluble secreted recombinant ENPP1 (srENPP1). The constructs included human albumin (hAlb) or human IgG Fc domain (hIgG Fc) to improve protein half life, and once included the ENPP2-somatomedin like domain (SMD). As shown in FIG. 2, the constructs were: CBA_srENPP1_Albumin, which contained (1) albumin secretory signal (hAlb sig) to signal the protein for secretion from the cell and (2) hAlbumin to stabilize the protein and increase its half-life.

CBA_srENPP1, which contained the albumin secretory signal but not a stabilizing fusion protein.

CBA_srENPP1_Fc, which contained ENPP1 cytosolic domain (CD, amino acids 1-76), ENPP2 signal sequence (SS, amino acids 12-30), with the extracellular ENPP1 domain fused with the IgG Fc domain for stabilization (srENPP1_Fc construct).

CMV_srENPP1_Albumin, which contained (1) albumin secretory signal (hAlb sig) to signal the protein for secretion from the cell and (2) hAlbumin to stabilize the protein and increase its half-life.

These constructs were validated in vitro using CBA and CMV promoters. Western blot analysis was performed in total lysates of human HepG2 liver cells transfected with the constructs and assessed by ENPP1 or Flag-tag antibody immunoblot. The srENPP1 with hAlbumin constructs showed striking expression of soluble ENPP1 for both CBA and CMV promoters in cell lysates. As shown in FIG. 4, there was superior expression of ENPP1 transgene in cells transfected with the CMV_srENPP1_albumin construct as compared to the CBA_srENPP1_albumin. Additionally, there was increased expression of rENPP1 in HepG2 cells using the CMV promoter construct compared to CBA promoter. As shown in FIG. 5, similar results were seen in human HEK293T cells. Expected molecular weights: Soluble secreted rENPP1=160 kD rENPP1=130 kD. Additional experiments in HepG2 cells showed inconsistent expression of srENPP1 in cell lysates and supernatant with the CBA promoter, and no increase in ENPP1 activity was seen in cell lysates or supernatant, so the CMV promoter was chosen for further experiments.

As shown in FIG. 6, the construct with srENPP1-hAlbumin and a CMV promoter showed expression of soluble, secreted srENPP1 in HepG2 (left) and HEK293 (right) cell supernatants, indicating proper secretion of srENPP1 into the extracellular space. The secreted protein was active; as shown in FIGS. 7A-B, the srENPP1-hAlbumin and CMV promoter construct showed significant ENPP1 activity in the supernatant of HepG2 and HEK293 cells indicating proper enzymatic function in addition to proper localization outside the cell. This was in contrast to the srENPP1_Fc construct for soluble recombinant ENPP1 Enzymatic activity of srENPP1_Fc (“plasmid #4”) was measured from the cell lysates (FIG. 8A) and supernatants (FIG. 8B) of HepG2 cells transfected with srENPP1_Fc construct. A significant increase in the ENPP1 activity was observed compared to cell lysates from untransfected cells but no increase in activity was observed in the supernatant of transfected cells, that the srENPP1_Fc construct has some cellular activity but no activity in the supernatant likely indicates it was not well secreted.

Table 1 provides a protein expression summary of soluble, secreted recombinant ENPP1 (srENPP1) constructs in HepG2 cells and HEK293T cells, indicating that SrENPP1 with hAlbumin and CMV promoter (CMV_srENPP1_Albumin) had higher expression compared to the CBA promoter.

TABLE 1
Expression levels

| | Cell- | Cell- | Supernatant- | Supernatant- | Cellular | Supernatant |
|---|---|---|---|---|---|---|
| Construct | ENPP1 | Flag | ENPP1 | Flag | activity | activity |
| CBA_srENPP1_Albumin | low | low | ND* | ND* | ND* | ND* |
| #1 | | | | | | |
| CBA_srENPP1 | ND | ND | ND* | ND* | ND* | ND* |
| #2 | | | | | | |
| CBA_srENPP1_Fc | Medium | ND | ND* | ND* | Medium* | ND* |
| #4 | | | | | | |
| CMV-srENPP1- | Medium | Medium | Medium | Medium | Low | high |
| Alb (#5) | | | | | | |

ND = not detected

### Example 2. Constructs for Delivering Transmembrane Recombinant rENPP1

Two DNA constructs were designed with the aim of delivering transmembrane recombinant ENPP1, as shown in FIG. 2, with the CMV or CBA promoter driving expression of a FLAG-tagged full length human recombinant ENPP1 (rENPP1). These constructs were also validated in vitro. As shown in FIGS. 4 and 5, transmembrane rENPP1 expression via CMV promoter was more effective than with the CBA promoter.

Table 2 provides a protein expression summary of recombinant transmembrane ENPP1 (srENPP1) constructs, indicating that the CMV promoter (CBA_rENPP1) had higher expression compared to the CBA promoter.

TABLE 2

| | Cell- | Cell- | Supernatant- | Supernatant- | Supernatant |
|---|---|---|---|---|---|
| plasmid# | ENPP1 | Flag | ENPP1 | Flag | activity |
| CBA_rENPP1 | Medium | ND | NA | NA | NA |
| CMV-rENPP1 | Medium | Medium | NA | NA | NA |
| (#6) | | | | | |

ND = not detected,
NA = not applicable given that it is a transmembrane protein

### Example 3. In Vitro Delivery of Soluble Recombinant ENPP1 Gene Construct Via Nanoparticle Packaging

Delivery of the CMV_srENPP1_hAlbumin construct was performed using four versions of nanoparticles (LNP18, LNP30, CHK18, and CHK30) in human HepG2 cells, and protein expression evaluated by Western blotting. The results demonstrated srENPP1 expression after delivery using CHK18 and CHK30 nanoparticles in cell lysates (FIG. 9A) that was comparable to commercial LTX lipofectamine reagent using a Flag-directed antibody, and in supernatant ENPP1 expression was shown to be comparable to commercial LTX lipofectamine reagent using an ENPP1 antibody and Flag antibody. (FIG. 9B).

ENPP1 catalytic activity assay was performed on the supernatant and total lysates of human HepG2 cells transfected with CMV_srENPP1_albumin using nanoparticles. As shown in FIG. 10, transgene delivery and activity in CHK18 nanoparticles was comparable with LTX lipofectamine. As shown in FIG. 11, transgene delivery and activity with CHK18 nanoparticles was higher than with LTX lipofectamine.

In addition, newborn pups were injected on day 3 with srENPP1 or rENPP1 expression plasmid packed in various nanoparticle preparations through retroorbital route. Tissue/organs were harvested on day 6 post injection and placed on fresh OCT blocks followed by cryosectioning and immunohistochemistry to study the expression profile of srENPP1 in various tissues and organs. FIGS. 12A-B show expression of srENPP1 (12A) or rENPP1 (12B) in different tissues and organs injected with different plasmid concentrations and nanoparticle preparations compared to control un-injected. An increase in tissue expression of srENPP1 was observed with increase in CMV_srENPP1_Albumin concentration. Among the different nanoparticles used in the study, LNP1-DOPE with 0.3 ug of CMV_srENPP1_Albumin showed increased expression in the aorta and liver without causing appreciable toxicity.

### Example 4. In Vivo Delivery of Soluble Recombinant ENPP1 Gene Construct Via Nanoparticle Packaging

Delivery of the CMV_srENPP1_hAlbumin construct was performed in 10 day old mice using C6 RH-647 nanoparticles using retroorbital injection of 20 uL (5 ug NPs). 6 days later, mice were sacrificed and fresh tissue samples in optimal cutting temperature compound (OCT) were analyzed by immunofluorescence. The results showed nanoparticle autofluorescence in aortic and small intestinal tissue, but it was not detectable in heart, kidney, or liver. Expression of srENPP1 was seen in endothelial cells of the small intestine, heart, liver, and kidney, and in the medial layer of the aortic tissue that colocalized with F-actin, indicating that the ENPP1 transgene was expressed in smooth muscle cells.

### Example 5. In Vitro rENPP1 Gene Delivery Via AAVPR Packaging

An in vitro assessment of AAVPR rENPP1 expression, activity, and inhibition of osteogenic VSMC phenotypic change was performed. As shown in FIG. 13A, AAVPR transduced human VSMCs more robustly than AAV9. As shown in FIG. 13B, juman aortic VSMCs treated with AAVPR rENPP1 at low and high doses demonstrated dose-dependent overexpression of ENPP1 at the protein level. As shown in FIG. 13C, increased rENPP1 activity was detected over time in VSMCs treated with AAVPR rENNP1 compared to untreated cells. In addition, AAVPR rENPP1 treatment decreased VSMC osteogenic phenotype switch: as shown in FIG. 13D, AAVPR rENPP1 treated cells demonstrated decreased calcification on Alizarin red staining compared to untreated cells after 21 days of culture in osteogenic media, and as shown in FIG. 13E) AAVPR treatment decreased migration of VSMCs compared to control-treated cells after 12 hours in osteogenic media. Finally, as shown in FIG. 13F, these phenotypic changes correlated with a reduction in RUNX2 protein levels with AAVPR rENPP1 treatment.

ENPP1 gene therapy was tested in vivo in ASJ^{-/-} mice. Compared to vehicle-treated GACI mice, GACI mice treated with pBAE srENPP1 or LNP1-DOPE srENPP1 showed improved survival with log rank p=0.03 and p=0.008, respectively (FIG. 13G).

The reduction of human VSMC calcification, migration, and RUNX2 protein expression with AAVPR rENPP1 supports the use of AAVPR rENPP1 as a therapy for a multitude of vascular calcification disorders where VSMCs undergo osteogenic phenotype switch manifested by increased calcification, migration, and RUNX2 expression. These vascular calcification disorders include calciphylaxis and cardiovascular disease including diabetic vascular calcification, ESRD-associated vascular disease, calcific aortic valve disease (CAVD), coronary atherosclerosis, coronary artery bypass graft (CABG) stenosis, in-stent stenosis, peripheral vascular disease, or cerebral atherosclerosis.

### Example 6. Regulation of Expression of srENPP1 with miR155 and miR122 Reduced srENPP1 Expression in a Dose Dependent Manner

To evaluate the ability of miR155 and miR122 to down regulate expression of an ENPP1 transgene, rENPP1 or srENPP1 was expressed from plasmid with constructs consisting of a CAG promoter upstream of rENPP1-FLAG; the constructs included miR155 or miR122 target sequences (three target sequences in tandem) were expressed in human aortic SMCs. The cells were transfected using LTX lipofectamine at 5 ul/ml with 1 ug/ml of the plasmid in normal media for three days. Expression of srENPP1 was evaluated by Western blot after being immunoprecipitated with an anti-FLAG antibody and run on a 4-12% gel with MOPS running buffer. The results, presented in FIGS. 14A-B, showed dose-dependent reductions in rENPP1 expression in the presence of miR122 or miR155, respectively (14A), and dose-dependent reductions in srENPP1 expression in the presence of miR155 (14B). As shown in FIG. 15, an activity assay showed decreased srENPP1 activity with an increase in miR155 that was consistent with the results of the Western blot.

### Example 7: Particle Screening for Delivery of Constructs to Liver Hepatocytes to Produce a Soluble Protein

The instant example describes screening of LNP composition(s) for efficient encapsulation of plasmid constructs. Screening was based on alteration of the cholesterol and DOPE lipid ratio while fixing the ionizable and DMG-PEG lipid amounts. Screening based on cholesterol and DOPE content was classified by high, medium, or low cholesterol amount as outlined in Table 3.

TABLE 3

| | Cholesterol Content | | |
|---|---|---|---|
| | High | Medium | Low |
| MC-3 | 50% | 50% | 50% |
| DOPE | 10% | 30% | 40% |
| PEG-DMG | 1.5% | 1.5% | 1.5% |
| Cholesterol | 38.5% | 18.5% | 8.5% |

The particles were screened for the delivery in liver hepatocytes (HepG2) cell line based on formulation composition varying in cholesterol and DOPE content illustrated in Table 3. It was found that a composition with high cholesterol was the most efficient in delivering a reporter plasmid expressing RFP, as well as delivering a plasmid encoding a soluble ENPP1 protein in liver hepatocyte cells in vitro (FIGS. 17A-C). FIG. 17A is a chart illustrating results of a flow cytometry experiment quantitating the percentage of RFP positive cells in the liver hepatocytes (HepG2) cell line. FIGS. 17B-C illustrate the detection of plasmid constructs expressing soluble ENPP1 and detection at cell lysates (FIG. 17B) and detection in cell media of ENPP1 protein expressed from constructs expressing the soluble protein.

The preferential delivery of an ENPP1 soluble construct in a mouse model for the generalized arterial calcification of infancy (GACI) disease (Asj mouse model (FIG. 18) was further demonstrated. This experiment demonstrated efficient soluble ENPP1 expression in the liver of Asj mouse models among other organs 6 days post injection. FIG. 18 provides histological images of liver of Asj mice injected with soluble ENPP1 plasmid (0.3 mg/kg) at day 3 (P3) express high levels of the enzyme (labeled red) in the liver, 6 days post injection, demonstrating in vivo expression of a construct delivered with the particles described in Table 3 to the liver.

The outcome of treatment of soluble ENPP1 constructs delivered with LNPs with high cholesterol content (see Table 3) was further evaluated. The particles and compositions used in the delivery provided measurable survival benefits extending the life expectancy of treated animals from 80 days (untreated animals) to 114 days (treated animals) (n=3). In addition, microCT scans showed reduced calcification in the vibrissae of treated diseased animals in comparison with untreated group (FIGS. 19A-D). FIGS. 19A-D depict results of LNPs delivery efficacy studies using plasmids expressing soluble ENPP1. FIG. 19A is an illustration presenting the injection regimen. Briefly P3 mice were injected at day 0, day 7, and day 14. FIG. 19B shows survival curves of animals. FIG. 19C is a chart illustrating animal body weight. FIG. 19D is a set of MicroCT scans to detect early development of calcification in treated (LNPs encapsulating soluble ENPP1, 0.3 mg/kg) and untreated animals.

The experiment demonstrated (1) the efficacy of the particles in delivering the plasmid construct encoding the soluble ENPP1 construct in vivo; and (2) the effectiveness of the treatment as measured by increased survival, reversal of calcification, animal body weight.

### Example 8: Particle Screening for Delivery of Therapeutic Cargos to Smooth Muscle Cells (SMCs)

Having established the therapeutic effect of a plasmid construct delivered with high cholesterol particles, particles that displayed select tropism to smooth muscle cells were screened in order to further improve delivery of the treatment.

To target nanoparticles to SMCs, a particle formulation based on the addition of a fifth component, DOTAP, a permanently cationic lipid (FIGS. 20A-B), was developed. It was reasoned that introducing DOTAP to the LNP composition at low percentage affects the chemical nature of the nanoparticles and increases the interactions with SMCs which is followed by cell internalization and construct expression. Importantly, a significant effect of the DOTAP with regards to particle size, size distribution, and zeta potential of the LNPs was not observed at the range that was screened (up to 10% DOTAP, FIGS. 20A-B).

Second, the transfection efficiency and expression of a model plasmid expressing RFP in mouse SMCs line (MOVAS) was evaluated. It was found that the presence of the permanently positive cationic lipid DOTAP, as well as the concentration of DOTAP, to be highly crucial for successful transfection of MOVAS cells. In contrast to the compositions incorporating DOTAP, the conventional four-component compositions of the art consisting only of ionizable lipids failed to demonstrate any expression. FIG. 21 is a pair of bar graphs depicting the results of the introduction of DOTAP lipid into the four-component formulation for screening its efficiency in delivering a plasmid cargo to MOVAS cells. As depicted in FIG. 21, the addition of as little as 1% DOTAP into the formulation drove an increase in cells expressing RFP (Left, the percentage of positive cells; right, the mean fluorescence intensity). In this experiment, MOVAS cells were incubated for 48 h with the reporter plasmid (2 μg/48 well plate) encoding RFP. Cellular expression was identified and measured using flow cytometry.

In addition to in vitro data, the role of DOTAP in the LNP formulation in the context of delivery of a plasmid nucleic acid encoding a therapeutic cargo to SMCs in vivo was confirmed. Using the Asj mouse model for the generalized arterial calcification of infancy (GACI) disease, successful delivery of plasmid DNA encoding the transmembrane ENPP1 expression at SMCs at the aorta (indicated by the white labeled SMC by F-actin) was demonstrated (FIG. 22, left column). Further, it was demonstrated that the conventional LNP formulations with four components do not demonstrate SMC expression in vivo (FIG. 22, middle column). The expression of the control LNPs of the art appear to be mainly localized at the adventitia. It was further confirmed that the described DOTAP formulations efficiently expressed a construct encoding a therapeutic gene, namely ENPP1 (FIG. 22, right column). In this experiment, the Asj mouse model at P3 was injected systemically with PBS (control), LNPs (conventional four-component formulation of the art), and DOTAP LNPs formulation (7% DOTAP) at 0.3 mg/kg plasmid dose. Histological images of the aorta show that ENPP1 expression (Flag-tag, top row) colocalized with SMCs marker expression (F-actin, bottom row).

Further, it was observed that the addition of a high percent of DOTAP (e.g. 50%) into the formulation increased tropism to the lung, which is undesired for smooth muscle cell delivery. In contrast, it was observed that the addition of DOTAP at relatively low percentages (<10%) enhanced uptake to SMCs without driving tropism to the lung (as our target in some cases is the liver). It was observed that the addition of DOTAP did not significantly affect the size of the formulated LNPs. However, the size distribution (PDI) increased with higher percentages of DOTAP, possibly due to the decreased proportions of the remaining lipids, which play a crucial structural role in particle formation. Furthermore, the zeta potential increased with higher DOTAP percentages, attributed to the substantial contribution of DOTAP quaternary amine head groups to the total surface charge of the nanoparticles. Despite varying percentages of DOTAP, encapsulation efficiency remained consistently above 90%, facilitating meaningful comparison between different formulations (FIG. 21).

The present disclosure provides the first demonstrated ability of this technology to deliver the claimed particles to smooth muscle cells, particularly to vascular smooth muscle cells (vSMCs).

### Example 9: Delivery of Gene Therapy Cargos Using Particles Targeting Smooth Muscle Cells (SMCs)

Further validation of the particles comprising the permanently cationic DOTAP component was sought by testing the delivery of different therapeutic cargos. In this experiment, an mRNA therapeutic cargo (as opposed to the plasmid therapeutic cargo of the aforementioned example) was first packaged into the DOTAP particles described above. As a threshold matter, it was determined that the nature of the nucleic acid therapeutic cargo (e.g., plasmid versus mRNA) appeared to have a minimum, insignificant effect on the size, size distribution, and zeta potential of the DOTAP LNPs encapsulating mRNA at the DOTAP ranges screened (up to 10%, FIGS. 23A-B). It was found that the DOTAP LNPs formulation increased the transfection of mRNA in VSMCs in vitro using the delivery of mRNA expressing GFP (FIGS. 24A-B). Moreover, in vitro studies using the MOVAS cell line demonstrated that increasing the content of DOTAP LNPs enhanced mRNA transfection efficiency in VSMCs, reaching an optimum at 10-20% DOTAP. Beyond this range, further increases in DOTAP content led to diminished mRNA delivery and reduced GFP expression (FIGS. 23A-B). Cell viability remained confirmed under experimental conditions, thus ruling out reduced expression due to cell death (FIG. 24C). 10% DOTAP LNPs also provided excellent delivery to primary human aortic cells cultured from an ACTA2 patient. (FIG. 24D).

To investigate the underlying cause for the observed optimal mRNA delivery, cellular uptake studies were conducted based on the hypothesis that DOTAP may enhance LNPs uptake owing to its positively charged head groups. MOVAS cells were incubated with LNPs or DOTAP LNPs encapsulating Cy5-labeled mRNA for 4 and 24 hours (FIG. 25A). At each time point, cells were visualized using a fluorescent microscope to qualitatively assess mRNA internalization and were subsequently harvested and analyzed by flow cytometry for quantitative evaluation.

The results indicated that the efficient mRNA delivery facilitated by 10% DOTAP LNPs was attributed to enhanced cellular uptake, evidenced by an increased Cy5 intracellular signal (FIG. 25B). Specifically, a 20-fold increase in fluorescent intensity was observed when compared to 0% DOTAP, and a 10-fold increase when compared to 100% DOTAP at the 4-hour time point. Notably, a significant increase was also observed at the 24-hour incubation time point (FIG. 25C). A 10% DOTAP formulation was used to investigate uptake mechanism for LNP cellular internalization, using Cy5-fluorescently labeled LNPs (mRNA-Cy5) in the presence of various inhibitors including chlorpromazine, chloroquine, rottlerin, EIPA, pitstop2, and Dynasore. Uptake was quantified using flow cytometry. The results showed that LNPs appear to utilize caveolin and macropinocytosis-mediated uptake into SMCs (FIG. 25D). LNPs with 10% or more DOTAP also provided excellent delivery to of plasmid DNA encoding RFP to MOVAS cells (FIG. 25E).

In addition to in vitro data, the role of DOTAP in the LNP formulation in the context of delivery of an mRNA encoding a reporter gene to SMCs in vivo was confirmed. In this example, the effect of DOTAP as a fifth component added to LNPs and its ability to deliver mRNA to SMCs in vivo in a Marfan disease mouse model was studied. Using DOTAP particles with a 7% concentration, mRNA expressing a Cre recombinase enzyme was delivered. The Cre recombinase enzyme enables the expression of tdTom protein in this genetically engineered model. It was found that when comparing the LNPs of the art to the instant LNPs comprising DOTAP, the same extent of tdTom expression is achieved in certain organs.

However, further histological analysis of the aorta revealed that the DOTAP formulation showed high expression in aorta SMCs, indicating SMC specificity in vivo (FIGS. 26A-B). FIGS. 26A-B illustrate the in vivo delivery of Cre-mRNA utilizing LNP and DOTAP LNP and expression of tdTom in the Marfan disease mouse model. Mice at P3 were injected with 1 mg/kg mRNA encapsulated in LNPs or DOTAP LNPs. FIG. 26A is a chart depicting expression of tdTom in different organs 6 days post injection. FIG. 26B comprises histology images of the aorta in which tdTom was expressed using DOTAP LNPs.

Increasing concentrations of DOTAP were also evaluated. FIGS. 26C-D show that increasing the % of DOTAP in LNP formulation increases SMC tdTom expression In vivo. In the experimental results shown in FIG. 26D, Cre-mRNA delivery utilizing LNPs with tested with increasing DOTAP content and expression of tdTom in a Marfan disease mouse model. Mice at P3 were injected with 1 mg/kg mRNA encapsulated in LNPs formulated with 0, 10, 50 and 80% DOTAP lipid. tdTom expression identified using immune fluorescence in histological sections of the aorta and localized to SMCs (indicated by α-SMA expression).

Thus, it was demonstrated that DOTAP (7%) comprising LNPs display preferential delivery towards smooth muscle cells in vivo, as compared to standard LNPs, in different mouse models and with two different cargos (plasmid and mRNA).

### Example 10: Targeting LNPs to Mutant Smooth Muscle Cells Using Targeting Peptides

In order to enhance the targeting, and potentially uptake, of the nanoparticles to specific tissue or cells DOTAP LNPs conjugated with peptides that can target smooth muscle cell tissue or cell surface receptors were developed. The targeting strategy was composed of two approaches. First, conjugating our DOTAP nanoparticles with peptides that target receptors highly expressed in diseased vasculature extracellular matrix (collagen IV) in order to increase the accumulation and the retention of the nanoparticles in diseased tissues. Second, conjugating our DOTAP nanoparticles with peptides that target receptors highly expressed on the surface of vSMCs (TL-6R, CD63, and GAL-3) increasing the uptake into these cells. A combination of the two approaches can potentially leverage both accumulation and retention at the target site and cell specificity. FIG. 27 is a schematic illustrating the aforementioned conjugation scheme. LNPs were conjugated with the desired peptide by swapping traditional PEG lipids typically present in standard LNP formulations with maleimide-terminally modified PEG lipid. Peptide conjugation was done in different densities controlled by the percentage of the maleimide-terminally modified PEG lipid in the LNP formulation, ranging from 0.15-1.2% (FIG. 27). Peptide sequences targeting extracellular matrix and cellular receptors are described in Table 4 below:

TABLE 4
Peptide sequences targeting extracellular
matrix and cellular receptors.

| Peptide receptor | Sequence |
|---|---|
| Col-4 | KLWVLPK-GGG-C; SEQ ID NO: 22 |
| IL6-R | C-GGG-LSLITRL; SEQ ID NO: 23 |
| CD63 | CRHSQMTVTSRL; SEQ ID NO: 24 |
| Gal-3 | C-GGG-ANTPCGPYTHDCPVKR; |
| | SEQ ID NO: 25 |

To evaluate this approach, LNPs conjugated with different peptides were first synthesized, their physical properties were characterized, and their functional activity in MOVAS cell line in vitro was examined.

Table 5 summarizes the physical properties of the assembled LNPs conjugated or non-conjugated with the listed peptides in changing surface densities controlled by the percentage of a linker lipid in the LNP formulation. Increasing linker percentages (0.15-1.2%) increases the conjugated peptides on LNPs surface which confirms the chemical conjugation to the surface of the LNPs. The physical properties highly affect the potential functionality of the LNPs therefore crucial to characterize. The physical properties of the LNPs post conjugation present a slight increase in size with no significant effect on PDI, however, not affected by the percent of conjugated peptide. On the other hand, zeta potential decreases while increasing the percentage of the conjugated peptide.

TABLE 5
Physical properties (size, PDI, and Zeta potential) of
peptide-conjugated and non-conjugated LNPs by Col4/IL6R/CD63/Gal3
targeting peptides in varying densities.

| | Size (nm) | PDI | Zeta potential (mV) |
|---|---|---|---|
| LNP-DOTAP | 131.8 | 0.14 | −5.48 |
| 0.15% NC | 145.2 | 0.15 | −5.47 |
| 0.3% NC | 161.1 | 0.24 | −4.24 |
| 0.6% NC | 137.8 | 0.17 | −4.17 |
| 0.9% NC | 136.3 | 0.19 | −4.39 |
| 1.2% NC | 118.6 | 0.19 | −5.89 |
| 0.15% Col-4 | 188.33 | 0.17 | −6.79 |
| 0.3% Col-4 | 144.20 | 0.26 | −7.00 |
| 0.6% Col-4 | 165.63 | 0.14 | −11.87 |
| 0.9% Col-4 | 148.83 | 0.19 | −14.33 |
| 1.2% Col-4 | 139.43 | 0.15 | −17.30 |
| 0.15% IL-6R | 131.97 | 0.25 | −7.66 |
| 0.3% IL-6R | 111.51 | 0.33 | −7.45 |
| 0.6% IL-6R | 60.24 | 0.57 | −9.78 |
| 0.9% IL-6R | 137.43 | 0.19 | −11.70 |
| 1.2% IL-6R | 140.30 | 0.07 | −13.90 |
| 0.15% CD-63 | 125.2 | 0.104 | 1.36 |
| 0.3% CD-63 | 120.2 | 0.047 | −3.67 |
| 0.6% CD-63 | 118.3 | 0.06 | −10.4 |
| 0.9% CD-63 | 195.1 | 0.24 | −13.5 |
| 1.2% CD-63 | 126.5 | 0.088 | −12.3 |
| 0.15% Gal-3 | 134.1 | 0.094 | −0.975 |
| 0.3% Gal-3 | 131.9 | 0.089 | −3.74 |
| 0.6% Gal-3 | 120.4 | 0.102 | −5.9 |
| 0.9% Gal-3 | 119.9 | 0.058 | −10.4 |
| 1.2% Gal-3 | 113 | 0.128 | −13.4 |

LNPs Decorated with Collagen IV (Col-JV)-Targeting Peptides

In addition, based on nanoparticle tracking analysis, the percentage of linker lipid was estimated and correlated to the concentration of conjugated peptide. Furthermore, the number of peptide units per LNP was plotted (FIG. 28) resulting in a range varying from about 194 peptides to about 1268 per LNP indicating the density of peptides per particle.

Following the synthesis and characterization, the efficiency of conjugated LNPs was first examined in functional assays in vitro. MOVAS cells were treated with peptide-conjugated LNPs encapsulating mRNA expressing GFP. GFP expression was analyzed by flow cytometry 24 h post treatment. The results in FIG. 29 demonstrate the extent of GFP expression as a function of conjugated peptide content and sequence. The efficiency of mRNA expression was highly affected by the peptide amount, demonstrating an optimum at 0.15-0.3% of conjugated peptide for in vitro application regardless of the peptide sequence. The addition of the lipid with the linker, even without the peptides, was also affected by the linker lipid content (non-conjugated), indicating that LNPs composed of high percentages of lipid linker affected their functional activity. It was then confirmed and a range was found for peptide conjugation that was not affected by the additional manipulations utilized for the purpose of peptide conjugation.

It was therefore optimized and confirmed that peptide-conjugated LNPs efficiently expressed GFP in vitro, suggesting that the conjugation did not moderate their functional activity, thereby encouraging the examination of the potential of targeting SMCs in vivo.

Following the in vitro validation of functional activity, we evaluated the ability of mRNA-loaded LNPs to selectively target VSMCs in vivo. The formulations shown in Table 6 were tested.

TABLE 6
LNP formulations.

| | | | | DMG- | DSPE- | |
|---|---|---|---|---|---|---|
| Formulation | MC-3 | DOPE | Cholesterol | PEG | PEG-mal | DOTAP |
| 0.15% NC | 0.450 | 0.0945 | 0.3420 | 0.0120 | 0.0015 | 0.100 |
| 0.3%-Col-IV | 0.450 | 0.0945 | 0.3420 | 0.0105 | 0.0030 | 0.100 |
| 0.3% NC | 0.450 | 0.0945 | 0.3420 | 0.0105 | 0.0030 | 0.100 |
| 0.6%-Col-IV | 0.450 | 0.0945 | 0.3420 | 0.0075 | 0.0060 | 0.100 |
| 0.6% NC | 0.450 | 0.0945 | 0.3420 | 0.0075 | 0.0060 | 0.100 |
| LNP/DOTAP/80% | 0.100 | 0.021 | 0.076 | 0.003 | 0.000 | 0.800 |
| 0.3%-Col-IV | 0.100 | 0.0210 | 0.0760 | 0.0120 | 0.0030 | 0.7880 |
| (80% DOTAP) | | | | | | |
| 0.6%-Col-IV | 0.100 | 0.0210 | 0.0760 | 0.0090 | 0.0060 | 0.7880 |
| (80% DOTAP) | | | | | | |
| 0.3%-IL-6R | 0.450 | 0.0945 | 0.3420 | 0.0105 | 0.0030 | 0.100 |
| 0.15%-Col-IV + | 0.450 | 0.0945 | 0.3420 | 0.0105 | 0.0030 | 0.100 |
| 0.15%-IL-6R | | | | | | |

The LNPs were designed to encapsulate nucleic acids and were conjugated with peptides targeting extracellular matrix components or cell surface receptors. This technology demonstrates improved targeting and gene expression in vSMCs in mouse models of vascular diseases, in part by targeting receptors in vSMCs.

To achieve this, we encapsulated Cre-mRNA in LNPs that were subsequently conjugated with a Collagen IV-targeting peptide, leveraging its affinity for the extracellular matrix of VSMCs. We hypothesized that Col-IV peptide conjugation would enhance LNP retention within the VSMC extracellular matrix, thereby increasing bioavailability and uptake by SMCs. To test this, Marfan mice were injected at P3 with Col-IV-conjugated LNPs, and tdTom fluorescence expression was analyzed six days post-injection. Additionally, we sought to determine the optimal peptide density on the LNP surface for maximal retention and subsequent SMC uptake, leading to tdTom expression (Table 6). We evaluated three peptide densities, 0.15%, 0.3%, and 0.6% relative to the lipid component PEG-DSPE, into which the peptide was conjugated. Histological examination of mice aorta revealed that LNPs conjugated with Col-IV at 0.3% peptide density exhibited the most efficient SMC-specific tdTom expression. Moreover, this enhanced expression was significantly higher compared to non-conjugated LNPs, reinforcing the effectiveness of Col-IV peptide-mediated targeting.

In addition, we have explored potential synergy between the two examined elements, peptide-based targeting and the addition of DOTAP lipid to the formulation, in increasing the targeting efficiency for SMCs. We formulated Cre-mRNA in a formulation containing 80% DOTAP lipid which was then conjugated by Col-IV targeting peptide and tdTom expression was examined 6 days post injection. FIG. 15 presents histology sections of the aorta demonstrating that there was no increase in tdTom SMC expression for the combined Col-IV-DOTAP approach in comparison to the single approach where we either use the optimal formulation (80% DOTAP) to target SMCs or decorate the LNPs with Col-IV targeting peptide. Leveraging two approaches to target SMCs doesn't demonstrate an increase in SMC tdTom expression.

The incorporation of DSPE-PEG-maleimide (DSPE-PEG-mal) lipid into the formulation was helpful, as it appeared to play a role in stabilizing the outermost shell of the lipid nanoparticle (LNP) and maintaining the presentation of the conjugated peptide.

To explore the potential of targeting inflamed tissues in adult animals we conjugated LNPs with peptides targeting IL-6R, which is overexpressed in situations where inflammation is involved. In addition, we designed an LNP formulation where the two peptides were conjugated to the same LNP as a means to enhance tissue targeting and cellular targeting at the same delivery strategy. Therefore, IL-6R targeting peptide was conjugated in combination with Col-IV targeting peptide. To test this hypothesis, we encapsulated mRNA expressing GFP and examined expression using histology imaging and analysis and injected three-month-old (adult) wild type mice and mice engineered with a gain of function mutation in SMAD4 to model the human condition, Myhre syndrome both fed a high fat diet. We found increased GFP expression in examined organs (aorta, kidney, liver) with the LNPs that were conjugated with both peptides in comparison to a single IL-6R peptide conjugation. It is important to note that even the wildtype mice had elevated inflammation due to the high fat diet the mice are fed, causing this difference to appear in some cases mainly in the liver and kidney, but not the aorta.

We verified the results obtained in the Myhre mouse model in an LDLR knockout mouse model, which is used as a model for inflamed vascular disease indications. GFP mRNA was encapsulated in LNPs conjugated by IL-6R targeting peptide (0.3%). 12 h post intravenous injection we examined GFP expression in the aorta using histology analysis. While comparing conjugated to non-conjugated formulation, we identified a significant increase in GFP expression using the IL-6R targeting peptide formulation, showing that the peptide sequence on the surface of the LNPs increased delivery of the encapsulated mRNA in smooth muscle cells.

### Example 11. Systemically-Delivered SMCs-Optimized LNPs Enable Efficient SMCs Gene Editing for ENPP1 Expression for GACI Gene Therapy

ENPP1^{asj/asj} mice were injected at P3 with SMC-optimized LNPs (10% DOTAP) carrying a plasmid encoding a FLAG-tagged wild type ENPP1, and aortic SMC ENPP1 expression was determined by detecting the FLAG-tag using TIC 7 days post injection. As shown in FIG. 30A, the 10% DOTAP LNPs provided robust expression. In addition, ENPP1 gene therapy was administered in ENPP1^{asj/asj} mice in three doses of LNPs (10% DOTAP) encapsulating plasmid expressing ENPP, and therapeutic efficacy was identified by vibrissae calcification imaged by microCT. As shown in FIG. 30B, a significant reduction in calcification was seen.

### Example 12: In Vivo rENPP1 Gene Delivery Via AAVPR Packaging

ENPP1 gene therapy was tested in vivo in ASJ^{-/-} mice, the gold-standard mouse model of GACI (Li et al., Dis Model Mech. 2013 Jun. 20; 6(5):1227-1235). Pups were injected on postnatal day 3 with a CAG-rENPP1 expression construct packed in AAVPR through the retroorbital route or with NPs containing DNA plasmid cargo expressing rENPP1.

Tissues and organs were harvested on day 7 post injection and placed on fresh OCT blocks followed by cryosectioning and immunohistochemistry to study the expression profile of rENPP1 in various tissues and organs. In vivo biodistribution of NPs and AAVPR was determined based on localization of fluorescent signals of ENPP1 expression in the aorta, liver, brain, heart, small intestine, kidney, and bladder. FIG. 31 shows expression of rENPP1 in different tissues and organs injected with a construct expressing rENPP1 packed in AAVPR as compared to control un-injected, demonstrating the highest levels of expression were in the aorta.

Micro-CT images were taken at 6, 8, and 12 weeks post-injection to evaluate levels of vibrissae calcification compared to untreated controls. As shown in FIG. 32A, ENPP1 gene therapy with either NP delivery of srENPP1 or AAVPR delivery of rENPP1 reduced vibrissae calcification on micro-CT. In addition, when post-natal day 3 Asj^{-/-} mice were injected retro-orbitally with increasing doses of AAVPR-CAG-rENPP1 (3e12 vg/kg, 1e13 vg/kg, 3e13 vg/kg), statistically significant reductions in calcification (P<0.004) were observed in both male FIGS. 32B-C and female mice FIGS. 32D-E at 6, 8, and 12 weeks post-injection across all dosages administered.

The effect of ENPP1 gene therapy on plasma pyrophosphate (PPi) levels was evaluated in GACI mice treated with poly-beta amino ester nanoparticle (pBAE) carrying CMV-srENPP1 or AAVPR delivering CAG-rENPP1, delivered via retro-orbital systemic injections. As shown in FIG. 33A, serum PPi levels were normalized in the mice treated with pBAE-CMV-srENPP1 or AAVPR-CAG-rENPP1. In addition, PPi levels were evaluated in ASJ^{-/-} mice that received 3 different doses of AAVPR-CAG-rENPP1 (low=3e12 vg/kg, intermediate=1e13 vg/kg, high=3e13 vg/kg). There was a dose-dependent increase in plasma PPi levels with AAVPR-CAG-rENPP1 treatment (FIG. 33B, trend ANOVA p=0.0012). Treatment with the intermediate and high dose AAVPR-CAG-rENPP1 restored plasma PPi levels to normal levels as measured in the untreated HET/WT group.

Survival of wild-type, heterozygous, and homozygous ASJ^{-/-} mice was also evaluated. A significant improvement (>150 days compared to a median survival of ˜79 days in the untreated group) in median survival of treated ASJ^{-/-} mice, as compared to untreated mice, was observed. See FIG. 34A-B. FIG. 34A are Kaplan Meyer curves of ASJ^{-/-} mice injected retro-orbitally at postnatal day 3 (P3) with 3 different doses of AAVPR-CAG-rENPP1 (low=3e12 vg/kg, intermediate=1e13 vg/kg, high=3e13 vg/kg) FIG. 34B ASJ^{-/-} mice injected retro-orbitally at postnatal day 14 (P14) with a lower dose of AAVPR-CAG-rENPP1 FIG. 34B. In both instances, there was improvement in survival after treatment with AAVPR-CAG-rENPP1 compared to no treatment (FIG. 34A=Log-rank p<0.001 for all doses vs. untreated; FIG. 34B=Log-rank p=0.012). In addition, the effect of ENPP1 gene therapy with the AAVPR-CAG-rENPP1 construct was evaluated in ASJ^{-/-} mice injected retro-orbitally with 3 different doses of AAVPR-CAG-ENPP1 (low=3e12 vg/kg, intermediate=1e13 vg/kg, high=3e13 vg/kg). Treatment with low dose of AAVPR-CAG-ENPP1 was associated with an improvement in weight compared to untreated mice, as shown in FIG. 35.

It was noted that survival of normal mice was not impacted by AAVPR-CAG-ENPP1 treatment at multiple doses or at a different age of injection, as depicted in the Kaplan Meier curves shown in FIG. 36A-B. Briefly, Wild-type (WT) and ASJ^{+/-} (HETs) were injected retro-orbitally with 3 different doses of AAVPR-CAG-ENPP1 (low=3e12 vg/kg, intermediate=1e13 vg/kg, high=3e13 vg/kg) FIG. 36A. Survival was similar to what would be expected of untreated WT and HET mice. WT and ASJ HETs were injected retro-orbitally at postnatal day 3 (P3) versus postnatal day 14 (P14) with the low dose of AAVPR-CAG-ENPP1 FIG. 36B. Survival was similar to what would be expected of untreated WT and HET mice. The same was observed with weight; there was no significant difference in overall weights between treated and untreated mice.

### Example 13: Treatment of Human Vascular Smooth Muscle Cells (VSMCs) with AAVPR-CAG-rENPP1

This example discloses a reduction in human vascular smooth muscle cell (VSMC) calcification after administering AAVPR-CAG-rENPP1. The method demonstrates a significant reduction in calcification, RUNX2 protein levels, and VSMC migration.

Briefly, human vascular smooth muscle cells (VSMCs) were treated with AAVPR-CAG-rENPP1 at doses of 8e9 vg/mL (low) and 1.6e10 vg/mL (high). The cells were cultured in osteogenic media for 21 days. Dose-dependent expression of the AVPR-CAG-rENPP1 construct was confirmed by Western blotting. See FIG. 37A. An enzyme assay as described above was used to measure ENPP1 activity in the treated human cells; AAVPR-CAG-rENPP1 treatment at both doses resulted in dose-dependent increase in ENPP1 activity compared to untreated cells. See FIG. 37B.

Alizarin red staining was used to assess calcification of human aortic SMCs, showing a significant reduction with AAVPR-CAG-rENPP1 treatment. FIG. 37C. Additionally, the physiological impact of the AAVPR-CAG-rENPP1 construct on calcification pathways was measured based on the activation of markers of the osteogenic phenotype. Briefly, RUNX2 is a master transcriptional regulator of the osteogenic phenotype switch of VSMCs that results in calcification across a whole host of disorders, including calciphylaxis, atherosclerosis, calcification related to chronic kidney disease and diabetes mellitus. RUNX2 protein levels and VSMC migration were reduced compared to controls treated with AAVPR-GFP. FIG. 37D. The osteogenic phenotype switch of VSMCs is associated with increased migration, which was reduced after treatment with AAVPR-CAG-rENPP1. FIG. 37E.

### Example 14. Performance of AAVPR in Human Vessels Ex Vivo

To evaluate the performance of AAVPR in transducing human vessels ex vivo, vessels were harvested from human limbs after amputation (FIG. 38A), then transduced with either AAVPR-GFP or AAV9-GFP and maintained in a perfusion bioreactor (FIG. 38B) as described above. The results showed that AAVPR had high transduction efficiency in healthy human vessels (FIGS. 39A-B).

The AAVPR-transduced vessels exhibited significant GFP signal in the medial layer with co-localization with a-SMA indicating strong transduction of vascular smooth muscle cells (FIG. 40A). Ex vivo perfusion of diseased human vessels with AAVPR-CBA-GFP resulted in significantly higher viral copy numbers and GFP expression compared to AAV9-CBA-GFP, indicating a greater vascular tropism with AAVPR than AAV9 (FIG. 40B).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A recombinant adeno associated viral vector (AAV) encapsulating a construct encoding an ectonucleotide pyrophosphatase/phosphodiesterase 1 (ENPP1) enzyme, said vector comprising:
an expression cassette comprising:
a 5′ ITR;
a promoter;
a nucleic acid encoding an ectonucleotide
pyrophosphatase/phosphodiesterase 1 (ENPP1) enzyme; and
a 3′ ITR; and
wherein the recombinant AAV vector comprises a capsid with a nucleic encoding peptide sequence PRPPSTH (SEQ ID NO:44) inserted therein.

2. The recombinant AAV vector of claim 1, wherein said promoter is a CMV immediate/early gene enhancer/CBA promoter (CAG).

3. The recombinant AAV vector of claim 1, wherein said nucleic acid encoding the ectonucleotide pyrophosphatase/phosphodiesterase 1 (ENPP1) enzyme is a full-length transmembrane ENPP1 enzyme.

4. The recombinant AAV vector of claim 3, wherein said full-length transmembrane ENPP1 enzyme comprises SEQ ID NO:1 or is at least 95% identical to SEQ ID NO:1.

5. The recombinant AAV vector of claim 1, wherein said expression cassette further comprises a regulatory sequence.

6. The recombinant AAV vector of claim 5, wherein said regulatory sequence comprises is a miRNA 155 (miR155) target sequence or a miR122 target sequence.

7. The recombinant AAV vector of claim 5, wherein said regulatory sequence comprises one, two, or three repeats of SEQ ID NO: 2 or SEQ ID NO: 106.

8. The recombinant AAV vector of claim 1, wherein said recombinant AAV vector is an AAV9 vector.

9. The recombinant AAV vector of claim 1, wherein a nucleic acid sequence encoding the PRPPSTH sequence is inserted into the capsid of said AAV9 vector in a position corresponding to immediately following amino acid 588 of a VP1 protein in the AAV9 vector.

10. A host cell transduced with the recombinant AAV vector of claim 1.

11. The host cell of claim 10, for use in a method of reducing calcification in a vascular cell.

12. The host cell of claim 11, for use in a method of reducing calcification in a vascular cell.

13. A nucleic acid construct for expression of an ectonucleotide pyrophosphatase/phosphodiesterase 1 (ENPP1) enzyme, said nucleic acid construct comprising:
a nucleic acid sequence encoding an AAV genome comprising:
a first inverted terminal repeats (ITR);
a nucleic acid encoding a replication (rep) sequence;
a nucleic acid encoding a capsid (cap) sequence, the nucleic acid encoding the cap sequence comprising a sequence encoding the peptide PRPPSTH inserted therein (SEQ ID NO:44);
a promoter;
a nucleic acid encoding an ectonucleotide pyrophosphatase/phosphodiesterase 1 (ENPP1) enzyme comprising a transmembrane domain; and
a second ITR.

14. The construct of claim 13, wherein said nucleic acid encoding the ENPP1 enzyme comprises the sequence of SEQ ID NO: 1.

15. The construct of claim 13, wherein said promoter is a CMV immediate/early gene enhancer/CBA promoter (CAG).

16. The construct of claim 15, wherein said nucleic acid comprises the sequence of SEQ ID NO: 75.

17. The construct of claim 13, wherein said expression cassette further comprises a regulatory sequence.

18. The construct of claim 17, wherein said regulatory sequence comprises one, two, or three repeats of SEQ ID NO: 2 or SEQ ID NO: 106.

19. The construct of claim 13, wherein said AAV genome is an AAV9 genome.

20. The construct of claim 19, wherein a nucleic acid sequence encoding a peptide having sequence PRPPSTH (SEQ ID NO:44) is inserted into the capsid of said AAV9 vector in a position corresponding to immediately following amino acid 588 of a VP1 protein in the AAV9 vector.

21. A particle comprising:
a) an amount of an ionizable lipid;
an amount of neutral lipid;
an amount of cholesterol;
an amount of one or more PEG-lipids; and
an amount of a DOTAP molecule;
b) a peptide conjugated to a linker in the particle; and
c) a construct for expression of ectonucleotide pyrophosphatase/phosphodiesterase 1 (ENPP1), comprising a sequence encoding an ENPP1 transgene, wherein the ENPP1 is (i) full-length human ENPP1 or (ii) a truncated version thereof comprising the extracellular domain of human ENPP1 (srENPP1) linked to a stabilizing protein, and a promoter that drives expression of the ENPP1 transgene.

22. The particle of claim 21, comprising:
a. about 78.8% of DOTAP;
b. about 10% of an MC3 ionizable lipid;
c. about 2.1% of a DOPE neutral lipid;
d. about 7.6% of cholesterol; and
e. about 1.5% of one or more PEG-lipids.


SEQUENCE LISTING (106 sequences)

SEQ ID NO: 1 - protein, 925 aa, Homo sapiens
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 2 - DNA, 20 bp, synthetic construct
  source 1..20: mol_type = other DNA
caattacgattagcactatc

SEQ ID NO: 3 - DNA, 60 bp, synthetic construct
  source 1..60: mol_type = other DNA
caattacgattagcactatccaattacgattagcactatccaattacgattagcactatc

SEQ ID NO: 4 - DNA, 82 bp, synthetic construct
  source 1..82: mol_type = other DNA
ctctggctaactagagaacccactgcttactggcttatcgaaattaatacgactcactatagggagacccaagctggctagc

SEQ ID NO: 5 - protein, 4 aa, synthetic construct
GGGS

SEQ ID NO: 6 - protein, 5 aa, synthetic construct
GGGGS

SEQ ID NO: 7 - DNA, 13 bp, synthetic construct
  source 1..13: mol_type = other DNA
gccgccaccatgg

SEQ ID NO: 8 - protein, 25 aa, synthetic construct
MGVLLTQRTLLSLVLALLFPSMASM

SEQ ID NO: 9 - protein, 19 aa, synthetic construct
MGWSCIILFLVATATGVHS

SEQ ID NO: 10 - protein, 21 aa, synthetic construct
MWWRLWWLLLLLLLLWPMVWA

SEQ ID NO: 11 - protein, 22 aa, synthetic construct
MDMRVPAQLLGLLLLWLRGARC

SEQ ID NO: 12 - protein, 16 aa, synthetic construct
MPLLLLLPLLWAGALA

SEQ ID NO: 13 - protein, 23 aa, synthetic construct
MDAMKRGLCCVLLLCGAVFVSPS

SEQ ID NO: 14 - protein, 18 aa, synthetic construct
MAFLWLLSCWALLGTTFG

SEQ ID NO: 15 - protein, 15 aa, synthetic construct
MNLLLILTFVAAAVA

SEQ ID NO: 16 - protein, 20 aa, synthetic construct
MYRMQLLSCIALSLALVTNS

SEQ ID NO: 17 - protein, 16 aa, synthetic construct
MKWVTFISLLFSSAYS

SEQ ID NO: 18 - protein, 20 aa, synthetic construct
MASHRLLLLCLAGLVFVSEA

SEQ ID NO: 19 - protein, 27 aa, synthetic construct
MARRSSFQSCQIISLFTFAVGVNICLG

SEQ ID NO: 20 - protein, 24 aa, synthetic construct
MALWMRLLPLLALLALWGPDPAAA

SEQ ID NO: 21 - protein, 24 aa, synthetic construct
MPSSVSWGILLLAGLCCLVPVSLA

SEQ ID NO: 22 - DNA, 24 bp, synthetic construct
  source 1..24: mol_type = other DNA
aagaatttccagagcagaagccac

SEQ ID NO: 23 - DNA, 24 bp, synthetic construct
  source 1..24: mol_type = other DNA
aagagaagaacccctgtgcgggag

SEQ ID NO: 24 - DNA, 24 bp, synthetic construct
  source 1..24: mol_type = other DNA
aagacctacgcctctatgcagtgg

SEQ ID NO: 25 - DNA, 24 bp, synthetic construct
  source 1..24: mol_type = other DNA
gatgatgacgacgacgatgactgc

SEQ ID NO: 26 - DNA, 24 bp, synthetic construct
  source 1..24: mol_type = other DNA
ttaatgctaatcgtgataggggtt

SEQ ID NO: 27 - DNA, 24 bp, synthetic construct
  source 1..24: mol_type = other DNA
aattacgattagcactatccccaa

SEQ ID NO: 28 - DNA, 22 bp, synthetic construct
  source 1..22: mol_type = other DNA
tagcttatcagactgatgttga

SEQ ID NO: 29 - DNA, 22 bp, synthetic construct
  source 1..22: mol_type = other DNA
atcgaatagtctgactacaact

SEQ ID NO: 30 - DNA, 22 bp, synthetic construct
  source 1..22: mol_type = other DNA
agcccctgcccaccgcacactg

SEQ ID NO: 31 - DNA, 22 bp, synthetic construct
  source 1..22: mol_type = other DNA
tcggggacgggtggcgtgtgac

SEQ ID NO: 32 - DNA, 22 bp, synthetic construct
  source 1..22: mol_type = other DNA
tgtaaacatcctacactcagct

SEQ ID NO: 33 - DNA, 22 bp, synthetic construct
  source 1..22: mol_type = other DNA
acatttgtaggatgtgagtcga

SEQ ID NO: 34 - DNA, 23 bp, synthetic construct
  source 1..23: mol_type = other DNA
tcatagccctgtacaatgctgct

SEQ ID NO: 35 - DNA, 23 bp, synthetic construct
  source 1..23: mol_type = other DNA
agtatcgggacatgttacgacga

SEQ ID NO: 36 - DNA, 21 bp, synthetic construct
  source 1..21: mol_type = other DNA
gcaggaacttgtgagtctcct

SEQ ID NO: 37 - DNA, 21 bp, synthetic construct
  source 1..21: mol_type = other DNA
cgtccttgaacactcagagga

SEQ ID NO: 38 - DNA, 21 bp, synthetic construct
  source 1..21: mol_type = other DNA
gtgttgaaacaatctctactg

SEQ ID NO: 39 - DNA, 21 bp, synthetic construct
  source 1..21: mol_type = other DNA
cacaactttgttagagatgac

SEQ ID NO: 40 - DNA, 18 bp, synthetic construct
  source 1..18: mol_type = other DNA
tcgaggagctcacagtct

SEQ ID NO: 41 - DNA, 18 bp, synthetic construct
  source 1..18: mol_type = other DNA
agctcctcgagtgtcaga

SEQ ID NO: 42 - DNA, 21 bp, synthetic construct
  source 1..21: mol_type = other DNA
cattattacttttggtacgcg

SEQ ID NO: 43 - DNA, 21 bp, synthetic construct
  source 1..21: mol_type = other DNA
gtaataatgaaaaccatgcgc

SEQ ID NO: 44 - protein, 7 aa, synthetic construct
PRPPSTH

SEQ ID NO: 45 - protein, 7 aa, synthetic construct
MAEPGAR

SEQ ID NO: 46 - protein, 7 aa, synthetic construct
MLYADNT

SEQ ID NO: 47 - protein, 7 aa, synthetic construct
SQDPSTL

SEQ ID NO: 48 - protein, 11 aa, synthetic construct
KLWVLPKGGGC

SEQ ID NO: 49 - protein, 11 aa, synthetic construct
CGGGLSLITRL

SEQ ID NO: 50 - protein, 15 aa, synthetic construct
CRHSQMTVTSRLGGG

SEQ ID NO: 51 - protein, 20 aa, synthetic construct
CGGGANTPCGPYTHDCPVKR

SEQ ID NO: 52 - DNA, 72 bp, synthetic construct
  source 1..72: mol_type = other DNA
atgaagtgggtaacctttatttcccttctttttctctttagctcggcttattccaggggtgtgtttcgtcga

SEQ ID NO: 53 - protein, 24 aa, synthetic construct
MKWVTFISLLFLFSSAYSRGVFRR

SEQ ID NO: 54 - DNA, 54 bp, synthetic construct
  source 1..54: mol_type = other DNA
atgaagtgggtaacctttatttcccttctttttctctttagctcggcttattcc

SEQ ID NO: 55 - protein, 18 aa, synthetic construct
MKWVTFISLLFLFSSAYS

SEQ ID NO: 56 - DNA, 81 bp, synthetic construct
  source 1..81: mol_type = other DNA
atggcaaggaggagctcgttccagtcgtgtcagataatatccctgttcacttttgccgttggagtcaatatctgcttagga

SEQ ID NO: 57 - protein, 27 aa, synthetic construct
MARRSSFQSCQIISLFTFAVGVNICLG

SEQ ID NO: 58 - DNA, 60 bp, synthetic construct
  source 1..60: mol_type = other DNA
atggcttctcatcgtctgctcctcctctgccttgctggactggtatttgtgtctgaggct

SEQ ID NO: 59 - protein, 20 aa, synthetic construct
MASHRLLLLCLAGLVFVSEA

SEQ ID NO: 60 - DNA, 2541 bp, synthetic construct
  source 1..2541: mol_type = other DNA
ataaatgttttgtagaataaaaaaaaaaaaagttcttcaaaagaaatctcaaatctccaaatggaaacaggtaaaagtggagctcccctggttccacggagaaccttttttgaggaaacttaggcaactcgcaggtaccttatgtcatgagacagagtttgaaaactacaattgactatctctaaatttcctcccaggtctaaaatgtgatgatagttactacttcagtacatcatccttaaggaaaattattaggtccacactgtttctatcctttgaattttacacataaattttgtaatcaaaagtttatttgtaatatcagatggaatcagataattgctttttgttttttccactgacaggaacataagattttgttgtgtagcttaagtcaaacgcagtttggaatatatattttttaaaaattgtaacttacatatccaaatacaatttttcaagaagtagagtattcagtagaaattaatctgtgaaagaagaggaattcagcagtggcctatttgatgaatgatttaacgtgcttatttcttccctttcatcaaaactctgtgtccccttgtttgccccctctgacttcatactctggagttgaccaagatccctcttccatcggattgttctgggaattttgaaataatctgctttttcctctctttcccctgttgcttctgatgccttagaattacattttcctcgctgatttagtttagaaaagagaaaagagcttccatgactagtagattatcacttttgggtttgctcttggaagtgacaagatgctaggatccctctttggaatgtaaaatttatctcttatatagaaaggatataaatgtagcaccagagactataaaactctgatactatctactgtactgtatagctgaacgccacaatgtgtctggtaatctattgactatcataaatgctatttctacagaaaagttaggaggtccatatttcgggcaaccaatgtatagctgaatgcagaacagtcatagttgggtactaaccatatatatgatttatccatcaacaggtgcatatgctcagaaattctgtatccataagaaatcagactactttcttttccttttgcaagtaaattgaatttagcctgagaggctgaggggaaattttcacatataagccacggttttgtgttttgtgttttgttttgtttatagatatagtactaactggatggatgcgataaaattcataggtggtactaagatacaataggatttgtgaaatggacaattgtcttgcataaatagcaagtaaaaaatcaagcctgtccttcataaaaattttattctggggtgtgcttgttttccaaaagtacctctgctaaatctcctgttagtcctgaaactagaaggcagaaaagcttctagtgctacagccaactgcagtgtagcctgagaaacaggcaacaaaaatagaacaccaggattgctgtgcgtgggtgaggcagaaaccacattatgagcaaaagcttccagtattattttagaaccaatacagagctctgtacttctctcctctctcttccaaaaacacatactacaaaaataaaatgaaatgaaatgtatgtgcatttgccctcttagaattatgattcttaattttttttcttgccttcctttctttggaagcgaatcgccagtatggaaacacagtgtgtaaagcaagcttcgagagaggaaagagttaattggttttaaggccctgcgatagagaattatggttggaaagatagaggctggacagctgggtttgctggggtatttttaaatgcattaatgcaggctccaatcactcggccatgcttgacctatttttggctcaggccgaccattgttctatttctgtgcctgtgggccatgctgttgttgattcatatgcaaatggattatcactcgctttagccaacttgagctgagagagactgagaaagggggaagagaggcacagacacagataggagaagggcaccggctggagccacttgcaggactgagggtttttgcaacaaaaccctagcagcctgaagaactctaagccaggtttaattggtttctttttctcgtgggtagacttaataattttctacgtattctgacaaagaaataaccccgaagcacgttcctatttcccacctgcttgtagtttccgggataacctaaactccagagagctatagcatccactctgtcctttctgctttgcacacaggttggtaacatgggaaaagtgtccaggtctttttaaaagtggatgcccatttgagcagaaaggaaatcattgtcgaagttgatcctctgctgcttctcctcagggaggagggagaaccagcgagggtagctcctggggccggtgcactgagcagtgatgaatgtttcatgtagctgaagtaagagtgactggaatatgctgcagacaatttacgagagtgactcctgtttttcctcagatggggtggctggacgagagcagctcttggctcagcaaaga

SEQ ID NO: 61 - DNA, 1350 bp, synthetic construct
  source 1..1350: mol_type = other DNA
ataaatgttttgtagaataaaaaaaaaaaaagttcttcaaaagaaatctcaaatctccaaatggaaacaggtaaaagtggagctcccctggttccacggagaaccttttttgaggaaacttaggcaactcgcaggtaccttatgtcatgagacagagtttgaaaactacaattgactatctctaaatttcctcccaggtctaaaatgtgatgatagttactacttcagtacatcatccttaaggaaaattattaggtccacactgtttctatcctttgaattttacacataaattttgtaatcaaaagtttatttgtaatatcagatggaatcagataattgctttttgttttttccactgacaggaacataagattttgttgtgtagcttaagtcaaacgcagtttggaatatatattttttaaaaattgtaacttacatatccaaatacaatttttcaagaagtagagtattcagtagaaattaatctgtgaaagaagaggaattcagcagtggcctatttgatgaatgatttaacgtgcttatttcttccctttcatcaaaactctgtgtccccttgtttgccccctctgacttcatactctggagttgaccaagatccctcttccatcggattgttctgggaattttgaaataatctgctttttcctctctttcccctgttgcttctgatgccttagaattacattttcctcgctgatttagtttagaaaagagaaaagagcttccatgactagtagattatcacttttgggtttgctcttggaagtgacaagatgctaggatccctctttggaatgtaaaatttatctcttatatagaaaggatataaatgtagcaccagagactataaaactctgatactatctactgtactgtatagctgaacgccacaatgtgtctggtaatctattgactatcataaatgctatttctacagaaaagttaggaggtccatatttcgggcaaccaatgtatagctgaatgcagaacagtcatagttgggtactaaccatatatatgatttatccatcaacaggtgcatatgctcagaaattctgtatccataagaaatcagactactttcttttccttttgcaagtaaattgaatttagcctgagaggctgaggggaaattttcacatataagccacggttttgtgttttgtgttttgttttgtttatagatatagtactaactggatggatgcgataaaattcataggtggtactaagatacaataggatttgtgaaatggacaattgtcttgcataaatagcaagtaaaaaatcaagcctgtccttcataaaaattttattctggggtgt

SEQ ID NO: 62 - DNA, 1191 bp, synthetic construct
  source 1..1191: mol_type = other DNA
gcttgttttccaaaagtacctctgctaaatctcctgttagtcctgaaactagaaggcagaaaagcttctagtgctacagccaactgcagtgtagcctgagaaacaggcaacaaaaatagaacaccaggattgctgtgcgtgggtgaggcagaaaccacattatgagcaaaagcttccagtattattttagaaccaatacagagctctgtacttctctcctctctcttccaaaaacacatactacaaaaataaaatgaaatgaaatgtatgtgcatttgccctcttagaattatgattcttaattttttttcttgccttcctttctttggaagcgaatcgccagtatggaaacacagtgtgtaaagcaagcttcgagagaggaaagagttaattggttttaaggccctgcgatagagaattatggttggaaagatagaggctggacagctgggtttgctggggtatttttaaatgcattaatgcaggctccaatcactcggccatgcttgacctatttttggctcaggccgaccattgttctatttctgtgcctgtgggccatgctgttgttgattcatatgcaaatggattatcactcgctttagccaacttgagctgagagagactgagaaagggggaagagaggcacagacacagataggagaagggcaccggctggagccacttgcaggactgagggtttttgcaacaaaaccctagcagcctgaagaactctaagccaggtttaattggtttctttttctcgtgggtagacttaataattttctacgtattctgacaaagaaataaccccgaagcacgttcctatttcccacctgcttgtagtttccgggataacctaaactccagagagctatagcatccactctgtcctttctgctttgcacacaggttggtaacatgggaaaagtgtccaggtctttttaaaagtggatgcccatttgagcagaaaggaaatcattgtcgaagttgatcctctgctgcttctcctcagggaggagggagaaccagcgagggtagctcctggggccggtgcactgagcagtgatgaatgtttcatgtagctgaagtaagagtgactggaatatgctgcagacaatttacgagagtgactcctgtttttcctcagatggggtggctggacgagagcagctcttggctcagcaaaga

SEQ ID NO: 63 - DNA, 434 bp, synthetic construct
  source 1..434: mol_type = other DNA
ataaatgttttgtagaataaaaaaaaaaaaagttcttcaaaagaaatctcaaatctccaaatggaaacaggtaaaagtggagctcccctggttccacggagaaccttttttgaggaaacttaggcaactcgcaggtaccttatgtcatgagacagagtttgaaaactacaattgactatctctaaatttcctcccaggtctaaaatgtgatgatagttactacttcagtacatcatccttaaggaaaattattaggtccacactgtttctatcctttgaattttacacataaattttgtaatcaaaagtttatttgtaatatcagatggaatcagataattgctttttgttttttccactgacaggaacataagattttgttgtgtagcttaagtcaaacgcagtttggaatatatattttttaaaaattgtaa

SEQ ID NO: 64 - DNA, 482 bp, synthetic construct
  source 1..482: mol_type = other DNA
cttacatatccaaatacaatttttcaagaagtagagtattcagtagaaattaatctgtgaaagaagaggaattcagcagtggcctatttgatgaatgatttaacgtgcttatttcttccctttcatcaaaactctgtgtccccttgtttgccccctctgacttcatactctggagttgaccaagatccctcttccatcggattgttctgggaattttgaaataatctgctttttcctctctttcccctgttgcttctgatgccttagaattacattttcctcgctgatttagtttagaaaagagaaaagagcttccatgactagtagattatcacttttgggtttgctcttggaagtgacaagatgctaggatccctctttggaatgtaaaatttatctcttatatagaaaggatataaatgtagcaccagagactataaaactctgatactatctactgtactgtatagctgaacgccaca

SEQ ID NO: 65 - DNA, 434 bp, synthetic construct
  source 1..434: mol_type = other DNA
atgtgtctggtaatctattgactatcataaatgctatttctacagaaaagttaggaggtccatatttcgggcaaccaatgtatagctgaatgcagaacagtcatagttgggtactaaccatatatatgatttatccatcaacaggtgcatatgctcagaaattctgtatccataagaaatcagactactttcttttccttttgcaagtaaattgaatttagcctgagaggctgaggggaaattttcacatataagccacggttttgtgttttgtgttttgttttgtttatagatatagtactaactggatggatgcgataaaattcataggtggtactaagatacaataggatttgtgaaatggacaattgtcttgcataaatagcaagtaaaaaatcaagcctgtccttcataaaaattttattctggggtgt

SEQ ID NO: 66 - DNA, 217 bp, synthetic construct
  source 1..217: mol_type = other DNA
gaaaactacaattgactatctctaaattttaaaatgtgatgatagttactacttcagtggtccacactgtttctatcctttggaacataagattttgttgtgtagcttaaggaagaggaattcagcagttgactatctcgattgttctgggaattttggaggctgaggggaaattttcacatataagcgctgggtttgctggggtattgagagtgac

SEQ ID NO: 67 - DNA, 2063 bp, synthetic construct
  source 1..2063: mol_type = other DNA
aagattatagttgtcatttgaacttggggataaaggagacatctatgacttggctggaaaagacagagctaatgtacattgcaaagcacatatttatagcaggaaaatgggaagatttctctttaattctggagatggagtggggatggggagagtagactactcattttaagggtgaaacattggaattcaacttgtttgatgttatattaattggtggttaattactaagctaagtacgtataaaacttttatctatggctagcttgtccccccaaagtcatgcaatatagtgaactggctttcgcactttaaattattcattgatcatgtaatgattcagatgattcatcttccaagatggacactgaaactaacactcatagtaggttgtggtttaaagagtggaacaaccgccagtctcattagtggaaattgtgatggttgaatttatcaaggatgaacatacacggtcttctttctgagattttctttaagattttcgcacagataatctatttcttaggttttggagagaaaacttgaattttattgatccctcagaactcaatctttcagatttcaaaggagctatttcttttaatggggactctgttaatatttataaaagctcttcacaggatggagggtgggagggaaactccatcccaacaagacaaaaagaatgaagcatgaggctccacctagttcatcactgctccttgaaatacatcagtattgaaagacacatccaccccacccccaacccagccctattgctgttccagctcaagagtcagaggtcccgaagctgtagctcttctacaatctgctgctctgtgacttcaagtctgttgtctgcaaagaaaactattgggttcccaagcaagagaggcacatctggtaggacagattttgtgattgcaaaagaagggggaaaaaaagaaagaaagaaaagacctctctatacaagataaccagaggcatcaaactgaaatcctcctgtggaaaataagctagtacttctgggcctgatggtgtagtgaaaacctgtgcttgaggatacattacagtgaaagagcaaagtgaatagtaagtagctattacttacctccttagggaggtgtgttgtttgtctgtacatcccccacagcacctagcacagtaccttgcatctcacctgccactcactaaaaagtctatcaagttagttaattatcgagacaacgccctcagaaatgagagaacagtaccctcttatccttgctgcactttccagcactgatacgctgcctaaaagaggactagggcacaggtttgaattaatgtcacaaaactggatgggcaagttacaacggtgttgattaaggaaacagaactcatggtgcaccggatatctccatcctgatgaacccttggaaaaatgccaaagatgcatatccccaggcaaatgcctgattagtctgggattgatagattggtctaggattcagccctactgggaagatgtctaaattataatcagtgtagaaagcgaagttctcctagaagaagaggcaaaggttaaaaagaagaaaagaaaagaaagtgaagtcctttctcccccaaaacctctcatcaatcaatcagggtaacaaacagaacactagggctctgtctgtggaccaaacccaaaagccctgcggtcagggccaggagggtagatcatgtgtttgtggcaacttcctctgtgggcttttgcccaggtctgtccccaagcatacgatggccaaaacttctgcaccagagcagcatcctgtgtaacacagtcaggtccagcagttagggaaaactgcccactcagagtagataatatctggaaggaatgactgtttgggaaaagttccaatgctagttcagtgccaacccttccccaccttctccagctctctcccactggttcctcccctctcaactgctctggttcttataaaaacctcacagccttccactaacatcccgtaggagcctctctccctactgctgctacacaagaccctgagactgacctgcaggacgaaacc

SEQ ID NO: 68 - DNA, 1675 bp, synthetic construct
  source 1..1675: mol_type = other DNA
cgccggggagcaggaaggccactcggcaccatatttagtcagggggagccggcagcccagagctggtatgcggcgctgggaattcctgcaggaaggagtccgcgcctgccctttttgggttgtctcccgcccgccgctcccgccgctcccggggagggggaccggcccggcccggcccggcccgggaacctcggaggagctggtgccgcgcggggagcggagcgcccgggctgcccgcgggtccccggcctggcgcggggccagcccaccgcctcgacttccttttatggcctgtgtgtgcgtgcgtggacaggagcggggagggagggacggggagaagacggagagcctggggaagagagagagagaaagcgcagagataggagtgagacacgcgggagagatggagagcaagagacacagagaccagagacaaagtgagacaggagggagagacagatacatcgacagatctagagaagcgagagggacagagacaaaagatagagcgagagacagcaatgatcagagtgacagacatgcagagacagtggcagagacagagcgagagagcctgtgatggagagagacagggaatgcaattttaggcgaggaatccttggggaagggaagttgttgaagggaactcgcagactctgggggcacacccactttctccttggatcttgacacttgcatcttgtaaataacgtaattatcaccgccaccgccttcccccattttgtagctatggacaccaagtctcagagaagtgaagtgacttgcccaaggtcacgcagctggcgagtggcgcacaggggagggggacagctgaaataatcacagtgggcttatttttaatttttatttgtattttggtcgtggtgatgtgggtggaggtggagatggcaagttgggaaaagtaaaaacttccccttcctgcacggttcccagcaagggtgggggcctcctgtcttgcactttgcaaagttcaagaaatcccctttccctacccttcacgctgcacagccggccctctttccagacagtgcgatgccaataaaatgggaagtggggtgggagatgtcaagtcagatccaccacagccccgacacggggaggaagaggttaaagcctttgcggccggaaccgactcagggaagacgttctcaagcatcccgcacagacactgcctgctcgaccccctttctctagggatccggagcgtctgcgaccgcctggggccggggctgagactcccgtccctgtgcgcacctgttccgtgcgcccttgtgcggtgcgcacctgttccgtgcacccttgtcccgagcgccccagctccttgcgctcccgccgggggtgcgccctgcagggggcgcggcgagggggccgcgagggaccctccccaactccaccccttcggcctcctcccctttcccagccgcgggcagctccgggtctataaagagaggcgtccgaggacgcgcagggagatttggacgctccggcctgggaggtgcgtcagatccgagctcgccatccagtttcctctccactagtccccccagttggagatctgtaagtagtagttgtcattctgggggcagattgcagggcaggggggtgttaaaagtcctatagggtattctataggggctggggtgcacttaggggt

SEQ ID NO: 69 - DNA, 2016 bp, synthetic construct
  source 1..2016: mol_type = other DNA
ctctcataatattgaagtagctttgttgtctatggtgacatccgaggttcttggtctcacggccacaaagatcaaatatgtggacacacacaaagggtgaggtttggagcagaatttttaataggcaaaaggaggagaatagctctgctacagagaggggtcccaaaaaatgggttgcaatacgtggtgaaatgcaggggtttttatagatgagctagtggggaggcggtgtctgatctacataaggagggaaaacccggtttcgtgcatatggcatgaaaacccggttaggaccaggtgtgtcatctgcatagggcttaagtgtctggcagccccggccccaatcttctactatataggcaggtagctactccgtgttgcttatttccttcttactgtgcatgtggtaaaaaagaggggaggtggaacccccatggtggacatgcctggccccagtagccccttctgtctgtgccgctgccggcatctcccgtgcaagcttccagtttccttacctatgtttgcagcccaatcttccaggttgctctttgttagaaaggaagtgatttcttgggctgctttttgttagaaggtaagttctgccgagggctcttttgctcatactatctgcctaaataatttctatctcctgtatcaatatcctatgtgtgactctattctgcatatatgacttaggttttaattattttgtttacgagttggtcctacccccccacccccgcttccaccacacttaaggaaagatacattttaattcattttttatatggtatgcatagttggaggccaaaagattgagttgaaggattcattacttgatgaataaatggatgggtggatggatgagtcagtataaatgaagagcatcccaagttgactattttaaagggataacatgcattttatatttgttaaacattcattctcatttgtaaacacttgtatatattcatgtattcaaaatgtattgaaaattaattgttttccttggtacctgaagtcagtcctgggtattgcaaagtagttcgaagtcactggtttccctctgtctcacttcactacacactggtgattttgctaagtgatgagccagaaaatgtacaagttccagaatcaaaggtaaaacaaaacccaccaagaatcagctcactgttagcgtcctagttgaaaaataaggcttatcaaagtatttcagattgcattaacacttttgatatagctaggtatcagtgtcaacctcaggagagggcaacgccaactcctgaaaccttcaatacagaaagtctagaaaaataagcaaatatacatacaaacaatagcgagaagactccagaaaccacataaaaccgtgggaatcgaaatatcccttacgtgtgttcagtacatgtgaacccacgtattttaagtggacgatttctctctcagagtaccgtaggtagtgggggacggggcgcagagggggagaaacagaaagtcgccttcctccatggttcatttgcatttccatccagaaactcacaggtcgaccccaagactccactctctcccgcctttgagaagccggaccggcatcggcggctgcatccttctcctcctccccgctctattttggggccccatgatctcatgccctctgcagaccacacgctgcaattccagcccagcccgcgccgcgaggccacgcagggcgattcctgcaagtgtcgggagggtggccggggcgcggggaggggacggcttggggggaagtttaagacacgcccacgtaagggacccaaaataaccgacacacagagtgcccgaaatcagacaggaagccaaataatccggggcgttgagtcgctttgccctgactgcgagagccgggtgtagggcggggagccaaggatctgaccgcgaggggcgggcgcggcggggaggggcggggcggggcgggcggcgcggggcctattaaaggcgcggccgggcagcggggccggagcggccggggccacg

SEQ ID NO: 70 - DNA, 2059 bp, synthetic construct
  source 1..2059: mol_type = other DNA
gaggtggaggctgcagtgagccgagatcgagccattgcactccagcctgggcaacaagagtgaaactccatctcaaaataaacaaataaacaaacaaaaacaaaaaaacaaagaaatagtggctgaggtcaactcttttggatattgggtaacaagcagatcaacttggctattacagcttttccccctgcccagctagtctgcatataagagaaaaataaatggatgatgttcagctcctgaaccctaactttttttccaagggaaccctcattgtcttctagctaggtttatacaatcaaatatcagacaaagtcaaaagttgttagactctggcaaggcaaaagaagaggctgtttttgtttttagaacatttagagcactgtggcatataatgaaataacaggattgccttgatatttttctttggggtttattgttttcctctatcatgtctggcttgtatcttgaaatttaacataaattataggaaaatctttcatggagttaacatcttttataagccttgctaaataccctactccaaatttgttcagtatggcatacagacaaaagcaagaggcttacatataaaaatgtataagcctgtcctctttgcaacagtgcaaaactaaaaggattttagaaaggcaattaacagtttggcagtatgtgtattttctttttttcaattagatattactagattctaagaatctgtaatgaaacccaggcctactgtacaaccccctataaacaagtttttgcttttgtgctatcatgttttatttgagcagagccactactctgttactaatttgttgcatggtaagtctcgtatttgtttacgcacgaacacatgtgctgcggaagaaaagatggtcactggacctgctgaggaagaggccaaaaacaaatctttttccctcatttgaatcctgaaggcatgtaatatttaaacaactttcagatttagagcccttgattttttcttttgaaagtgtgctggttgccaaattgcttagtaaagaaactttagtacccacaatagcctcaaaggacacagtatttggtggctacatttaaccaagaaataactagggaaacaattcttgacatttcaattcttgctgccattgagatttccttcctaaattcaattgctaataaagcttcccatgccatccccttcccccacccatctgcctctcaaaaaccgccccctcctccgccccgcccgaaacaagctgacagtctttacgggaagaagaggcagagactgaagttattttgtccgtcctcccagggcagaaggactttatgctgcgtggctggcatttccagtattttgataaaagttacttctctaaacttgaaccacagcatggggaaaataatgttatcttttatgttagttacaattttgcgaaatttcatcatgagaaactttcaaggaggcagaagtagctgagaatgtgatactagggacagggtcgctcaaactgccagcaaaataaatagatgggaaagacaggaacgttattaggtttataaatgataaaactttccttgcttaaaaaaataaagtggtgaggttgacgccattgaatgactgcattgtaaaccttaaaagcttaaagctggtggaaagcccttgcacagccctgttttcatttttatcggatgaaatgcatttggtttctttttgacaaaccatgtttttgtgaggaaaggaaaatgaacatgcactgttatgggccacggcaatgtaacaaaccctcctgttgccagaggataccacatgaaagtgtctgtgggttaggggagggacctgtaagggggcggggataagggggatgatagcttaagcctcttaggctcagagctgcgatttgtgaacaccctgtgatgtaatcaagctctggacaaatcagaggagtctgtcaacctcccaggtgggattgcttatagttaatagactaaacccagagcctcaaagcagtgcactccgtgaaggcaaagagaacacgctgcaaaaggctttccaagaatcctcgac

SEQ ID NO: 71 - DNA, 2328 bp, synthetic construct
  source 1..2328: mol_type = other DNA
gggaattacctgtagattattattaggagaaaaaaagtgctaattaatgaacagtgtacatggtatatgatcatgctataaaagtgtttctatatgcataaaatacttctagaagaaaacataacctgaggatattggctacagacagaaaaaataacaggtaagagaagagaaagagactagttattggatccacccttttatacctgtgcattttgaaccatataaatgtattacctattagaaaacaactaaacaaaaaggttaaaacatgactgtattggcattatatgatccatgtctgagtggcaaatcttatcaccagtggctgacacgcaaacagttctcatttccctacctatagtgtaccatgagaaggtaaggcactactgcaatgtgagctcttcgaggatgacacatttttgtctttttcacggatacattttctggtagctgttctttgttacttttacagtttgtctcctcatttttactataatattttcctatccttattatagaatcttggtattcctattgtagcatctttgtatcaatgctgtgccaattcctttatttagcagaaaaatggaatttgtatccttgacaagtaaagcttaacatatctgttttcaagtgtaccaaataaatccgaataggatgaaaatgatctcaattaatctaacataggggaggtttttttttttaaacatctcacactgtgcagtatcaacttgacttctcaacacaggttttccactgaaatatacaaattaaaacaaatggaagccagaataaaaaatgataacaattttatttctcccttctcaggaagatcttaagagcagcatgaaagtaaatgttaaaagaaaaaaaaaaaaaaaaagaagaggccaaaaatcatcagctgaatgtattaaaaatgaagcgctcacctgagaaagaccaccccaaaactgtctgaaggcccccaaacagctaattagttttgttttctcatcttcaggagtatccataaacatgctaaaaaaataaaaattacattatttgtcattcgttttaaaaaggtaattacacacaatcagaatagtggatcttataaccttccaacacgaaactaagtaaaatctgacagacacaatcacataaaaacacaaaactctgccacgcatacactcacccaggaaaagcctcttctataacttccgttttctgtaaaaaaaaaaaaaacaaaatgtaaacaatgttaattttaaaaaggcgcctcccttagccaagtattacaatgtaacacagggcaagtcaccttaatcagctctgaaactatcaatacctgtctggcctgtcactgtatcgatttcaaaggatacaatgaacttgacagcagtttgagaatcactaaacttccccgcatacaacctcggtgtcaaaacaagggaataaaaagggaggcgtgagaggaggttgcccaggccagtttccttggtcggctgcccccgcgtctccccgtccccaagccgtagtcctggatggccggcagctgcactcaccaccacctcttcgaaaatgctctgcagttgcgtctccatctgtactatcacccccgcctttccagggtgcccggcaaggcccggatcagactcgagctctgggaatataggggcagaggggcggagacctctggaggaaaccgtagctcctcggcgtcgcttcctcccccagcgctttacctggagcgttccctcccgagcccagccaacagcaggaacctgtacggaagacgggaagggcccggtacgcgccgtttgcaaaccccgcagaaaccagcggcgccaccagaaggttccgtctgtggagaagggcggcccgcaagccggacgagagcgcccccaaacggaaccttgagcccaaggaccccggaggcatcgtcgaccgcaggccgccacctcccggctggagaaggagttgcttcctgttcggggctaacccgcttgcagactgagaaatgaaaagtttggaacccagaagctgcagcctgcggaaaatgctggagccgcagccgacaccaagttgatgtctgtgagcagctgaccttctggacctccatcagcggcctgcagccaggagctgaagccacagagacggtgtcttctcagctatgataacctcagtgcaaatattgcccaatcactgaagagctgtgatcttcctaaacagttaaaagtcaggcacagctatgtaactcatacagtttctctttgccagactagactaaagaaggagcactaatttattctgataaaacaggtctatgcagctaccaggaca

SEQ ID NO: 72 - DNA, 279 bp, synthetic construct
  source 1..279: mol_type = other DNA
tcgaggtgagccccacgttctgcttcactctccccatctcccccccctccccacccccaattttgtatttatttattttttaattattttgtgcagcgatgggggcgggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 73 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 74 - DNA, 210 bp, synthetic construct
  source 1..210: mol_type = other DNA
agttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcac

SEQ ID NO: 75 - DNA, 584 bp, synthetic construct
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 76 - DNA, 1000 bp, synthetic construct
  source 1..1000: mol_type = other DNA
tgtgtgtgtgtgttttaattttctttatggaaaaattgacaaaaaaaaaatagagagagaggtatttaactgcaataaactggccccatgtggcccccgccttgtctgcttgtgtgtttgtccatctcaggagtggggagggggcctggggtctgcagagctccacgaggcatggttctgctgttgtgcacatggctgtgcatggtccctgccagctgcaccacccattacccagtggttggttggatggatggaggaattaaggaatgaatgtcccctttgaggccctagacgtgcatgagggtgtggggagctggggtcaaggacatgtcccatgttggaggagaggcaggggtctccgtgtcaacagttcctgaaaacacaaccagcccctggccctgccctgctgggccaaagccctcccctctgcaccagccaatagtggggcctggccttgagcccctcacccccagggagggcagatggccagggcgccaagcttggcccgtcagcctgtcgccttgcaccaaggctctggcgcctgtgctgtgacccctgcccctgctgatgatgaaacctgtcctcagctgagatgcagcgatgcctggtagggctgggggctgctcctgtgtctccccaggtgagcacacccctattcactgggccctgcttcagcctgcagcacccttcaactcccaggagctgggcttgccactctgctcaccttgtggagctccatctgcctttcctccccaattcccccactccctgcactcgtctcttcccacaagagccctgtctccttttcctagctattcccatctgaggccatctttattcatttagtttttagagacagggtttcactctcacccaggctggggtgcagtggcacacaatcacggctcactgcagccttgaccaactacaggtgcgtagcaccacagccaagtttttgtatagatggggtctcgctttgttacccaggctgtgacaagag

SEQ ID NO: 77 - DNA, 2075 bp, synthetic construct
  source 1..2075: mol_type = other DNA
agagcgtctccatgctatggttgcatttccgttttctatgaatgaatttgcattcaataaacaaccagactcagttcttggggcccttgtttgcactccctctgggtggagctgttgaggatgaggggagaggcggaggtcttccatttccccattcttcaagccatggccctactgggaactgcaattccttgattctcccgtttttcctgtccctccagcaacagcattaattcagtaaacatttaccggggcactgtgctggacagaggccagttcctggaaaagcctttcccacgccatcccactgcagacatccctccttacctccccaggaacagcagtctctgcccacctggccccgcccaccagactgaggctcacttcacctctgacctgagcggcccccagctcaccaagccacaggcccaagcagtgctccctgatgcggcgtttataatccgctcagcgtgcaggccgaggcaggagggtgatgaaagctgggcaggctccaagaggagggagttttgatatgtccctgaaagattcatttagacttcagtcggctaaggaggacatgatttgggggccaaggaatctgttgaattcagaacacaaccagaggtctgcagggtcagggatggaggagtgggctttcccctcgccagggcccactcctcttcctgcttttcctgcaggcgccactgggaggtgctatggctgtgcctcccctgggctctggagcatgtccagttgcagtgggcagaactgcggaggcgggcccctcctctgccaggcctggcagccccctcctagggccttgtttggctaggggtggtgccgggtgtggcagtgtgtgtgtagtggagagtgttaggtcttccctaccagatgcccttgcaggggagtgccacagcagtcagtccagggatcccactgttagtctcaccttttttaacctcttatctctccccaagatccctgaagccaggtacgagcaagatgagagtgggttatctctggagtgacagaggctggtctgttttccaggctggtagggactgttcctaaagggaggaagggatgataccagcctcctgagcctccttctcctgcgttagtgtctcaggccctgccaggccttatagaccctcttattgacactgcccactggatggggaccggagttggactcagcttctgccgaaccctcaaatcccagccccaactaaagcatataactcaagacctacctgcactgaaagctcttctcaacctgagcagggtggtccaattgaaagggtgggtctgaccacctctcctgcacccatgcgggttggcagaggtgtgcaggatctgccacttaccattcaccatgtggccttgaggaagacgcactcggggcctcagtttcctcatctataaaatggggatgtaattacaccctcacactgtagctgtgagtattcaatgagagcactgcaaagggcctggtgtggagtaggtcctcaggaaaggttggatcccatgtcccatcagagctaaaagccccaggaggagagggtggctggtttgtccccacaaacccctgggattcccggctccccagccccttgcccctctctccagccagactctattgaactccccctcttctcaaactcggggccagagaacagtgaagtaggagcagccgtaagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctagcagggagggggccgaggagcgagccagtgggggaggctgacatcaccacggcggcagccctttaaacccctcacccagccagcgccccatcctgtctgtccgaacccagacacaagtcttcactccttcctgcgagccctgaggaagccttctttccccagac

SEQ ID NO: 78 - protein, 8 aa, synthetic construct
KNFQSRSH

SEQ ID NO: 79 - protein, 8 aa, synthetic construct
KRRTPVRE

SEQ ID NO: 80 - protein, 8 aa, synthetic construct
KTYASMQW

SEQ ID NO: 81 - protein, 8 aa, synthetic construct
DDDDDDDC

SEQ ID NO: 82 - DNA, 8223 bp, synthetic construct
  source 1..8223: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatggagcgcgacggctgcgcggggggcgggagccgcggcggcgagggcgggcgcgctccccgggagggcccggcggggaacggccgcgatcggggccgcagccacgctgccgaggcgcccggggacccgcaggccgcggcgtccttgctggcccctatggacgtgggggaggagccgctggagaaggcggcgcgcgcccgcactgccaaggaccccaacacctataaagtactctcgctggtattgtcagtatgtgtgttaacaacaatacttggttgtatatttgggttgaaaccaagctgtgccaaagaagttaaaagttgcaaaggtcgctgtttcgagagaacatttgggaactgtcgctgtgatgctgcctgtgttgagcttggaaactgctgtttagattaccaggagacgtgcatagaaccagaacatatatggacttgcaacaaattcaggtgtggtgagaaaaggttgaccagaagcctctgtgcctgttcagatgactgcaaggacaagggcgactgctgcatcaactacagttctgtgtgtcaaggtgagaaaagttgggtagaagaaccatgtgagagcattaatgagccacagtgcccagcagggtttgaaacgcctcctaccctcttattttctttggatggattcagggcagaatatttacacacttggggtggacttcttcctgttattagcaaactaaaaaaatgtggaacatatactaaaaacatgagaccggtatatccaacaaaaactttccccaatcactacagcattgtcaccggattgtatccagaatctcatggcataatcgacaataaaatgtatgatcccaaaatgaatgcttccttttcacttaaaagtaaagagaaatttaatcctgagtggtacaaaggagaaccaatttgggtcacagctaagtatcaaggcctcaagtctggcacatttttctggccaggatcagatgtggaaattaacggaattttcccagacatctataaaatgtataatggttcagtaccatttgaagaaaggattttagctgttcttcagtggctacagcttcctaaagatgaaagaccacacttttacactctgtatttagaagaaccagattcttcaggtcattcatatggaccagtcagcagtgaagtcatcaaagccttgcagagggttgatggtatggttggtatgctgatggatggtctgaaagagctgaacttgcacagatgcctgaacctcatccttatttcagatcatggcatggaacaaggcagttgtaagaaatacatatatctgaataaatatttgggggatgttaaaaatattaaagttatctatggacctgcagctcgattgagaccctctgatgtcccagataaatactattcatttaactatgaaggcattgcccgaaatctttcttgccgggaaccaaaccagcacttcaaaccttacctgaaacatttcttacctaagcgtttgcactttgctaagagtgatagaattgagcccttgacattctatttggaccctcagtggcaacttgcattgaatccctcagaaaggaaatattgtggaagtggatttcatggctctgacaatgtattttcaaatatgcaagccctctttgttggctatggacctggattcaagcatggcattgaggctgacacctttgaaaacattgaagtctataacttaatgtgtgatttactgaatttgacaccggctcctaataacggaactcatggaagtcttaaccaccttctaaagaatcctgtttatacgccaaagcatcccaaagaagtgcaccccctggtacagtgccccttcacaagaaaccccagagataaccttggctgctcatgtaacccttcgattttgccgattgaggattttcaaacacagttcaatctgactgtggcagaagagaagattattaagcatgaaactttaccctatggaagacctagagttctccagaaggaaaacaccatctgtcttctttcccagcaccagtttatgagtggatacagccaagacatcttaatgcccctttggacatcctataccgtggacagaaatgacagtttctctacggaagacttctccaactgtctgtaccaggactttagaattcctcttagtcctgtccataaatgttcattttataaaaataacaccaaagtgagttacgggttcctctccccaccacaactaaataaaaattcaagtggaatatattctgaagctttgcttactacaaatatagtgccaatgtaccagagttttcaagttatatggcgctactttcatgacaccctactgcgaaagtatgctgaagaaagaaatggtgtcaatgtcgtcagtggtcctgtgtttgactttgattatgatggacgttgtgattccttagagaatctgaggcaaaaaagaagagtcatccgtaaccaagaaattttgattccaactcacttctttattgtgctaacaagctgtaaagatacatctcagacgcctttgcactgtgaaaacctagacaccttagctttcattttgcctcacaggactgataacagcgagagctgtgtgcatgggaagcatgactcctcatgggttgaagaattgttaatgttacacagagcacggatcacagatgttgagcacatcactggactcagcttctatcaacaaagaaaagagccagtttcagacattttaaagttgaaaacacatttgccaacctttagccaagaagacgactacaaagacgatgacgacaagtgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 83 - DNA, 8223 bp, synthetic construct
  source 1..8223: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgctctggctaactagagaacccactgcttactggcttatcgaaattaatacgactcactatagggagacccaagctggctagcgccaccatggagcgcgacggctgcgcggggggcgggagccgcggcggcgagggcgggcgcgctccccgggagggcccggcggggaacggccgcgatcggggccgcagccacgctgccgaggcgcccggggacccgcaggccgcggcgtccttgctggcccctatggacgtgggggaggagccgctggagaaggcggcgcgcgcccgcactgccaaggaccccaacacctataaagtactctcgctggtattgtcagtatgtgtgttaacaacaatacttggttgtatatttgggttgaaaccaagctgtgccaaagaagttaaaagttgcaaaggtcgctgtttcgagagaacatttgggaactgtcgctgtgatgctgcctgtgttgagcttggaaactgctgtttagattaccaggagacgtgcatagaaccagaacatatatggacttgcaacaaattcaggtgtggtgagaaaaggttgaccagaagcctctgtgcctgttcagatgactgcaaggacaagggcgactgctgcatcaactacagttctgtgtgtcaaggtgagaaaagttgggtagaagaaccatgtgagagcattaatgagccacagtgcccagcagggtttgaaacgcctcctaccctcttattttctttggatggattcagggcagaatatttacacacttggggtggacttcttcctgttattagcaaactaaaaaaatgtggaacatatactaaaaacatgagaccggtatatccaacaaaaactttccccaatcactacagcattgtcaccggattgtatccagaatctcatggcataatcgacaataaaatgtatgatcccaaaatgaatgcttccttttcacttaaaagtaaagagaaatttaatcctgagtggtacaaaggagaaccaatttgggtcacagctaagtatcaaggcctcaagtctggcacatttttctggccaggatcagatgtggaaattaacggaattttcccagacatctataaaatgtataatggttcagtaccatttgaagaaaggattttagctgttcttcagtggctacagcttcctaaagatgaaagaccacacttttacactctgtatttagaagaaccagattcttcaggtcattcatatggaccagtcagcagtgaagtcatcaaagccttgcagagggttgatggtatggttggtatgctgatggatggtctgaaagagctgaacttgcacagatgcctgaacctcatccttatttcagatcatggcatggaacaaggcagttgtaagaaatacatatatctgaataaatatttgggggatgttaaaaatattaaagttatctatggacctgcagctcgattgagaccctctgatgtcccagataaatactattcatttaactatgaaggcattgcccgaaatctttcttgccgggaaccaaaccagcacttcaaaccttacctgaaacatttcttacctaagcgtttgcactttgctaagagtgatagaattgagcccttgacattctatttggaccctcagtggcaacttgcattgaatccctcagaaaggaaatattgtggaagtggatttcatggctctgacaatgtattttcaaatatgcaagccctctttgttggctatggacctggattcaagcatggcattgaggctgacacctttgaaaacattgaagtctataacttaatgtgtgatttactgaatttgacaccggctcctaataacggaactcatggaagtcttaaccaccttctaaagaatcctgtttatacgccaaagcatcccaaagaagtgcaccccctggtacagtgccccttcacaagaaaccccagagataaccttggctgctcatgtaacccttcgattttgccgattgaggattttcaaacacagttcaatctgactgtggcagaagagaagattattaagcatgaaactttaccctatggaagacctagagttctccagaaggaaaacaccatctgtcttctttcccagcaccagtttatgagtggatacagccaagacatcttaatgcccctttggacatcctataccgtggacagaaatgacagtttctctacggaagacttctccaactgtctgtaccaggactttagaattcctcttagtcctgtccataaatgttcattttataaaaataacaccaaagtgagttacgggttcctctccccaccacaactaaataaaaattcaagtggaatatattctgaagctttgcttactacaaatatagtgccaatgtaccagagttttcaagttatatggcgctactttcatgacaccctactgcgaaagtatgctgaagaaagaaatggtgtcaatgtcgtcagtggtcctgtgtttgactttgattatgatggacgttgtgattccttagagaatctgaggcaaaaaagaagagtcatccgtaaccaagaaattttgattccaactcacttctttattgtgctaacaagctgtaaagatacatctcagacgcctttgcactgtgaaaacctagacaccttagctttcattttgcctcacaggactgataacagcgagagctgtgtgcatgggaagcatgactcctcatgggttgaagaattgttaatgttacacagagcacggatcacagatgttgagcacatcactggactcagcttctatcaacaaagaaaagagccagtttcagacattttaaagttgaaaacacatttgccaacctttagccaagaagacgactacaaagacgatgacgacaagtgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 84 - DNA, 9314 bp, synthetic construct
  source 1..9314: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttcgccggggagcaggaaggccactcggcaccatatttagtcagggggagccggcagcccagagctggtatgcggcgctgggaattcctgcaggaaggagtccgcgcctgccctttttgggttgtctcccgcccgccgctcccgccgctcccggggagggggaccggcccggcccggcccggcccgggaacctcggaggagctggtgccgcgcggggagcggagcgcccgggctgcccgcgggtccccggcctggcgcggggccagcccaccgcctcgacttccttttatggcctgtgtgtgcgtgcgtggacaggagcggggagggagggacggggagaagacggagagcctggggaagagagagagagaaagcgcagagataggagtgagacacgcgggagagatggagagcaagagacacagagaccagagacaaagtgagacaggagggagagacagatacatcgacagatctagagaagcgagagggacagagacaaaagatagagcgagagacagcaatgatcagagtgacagacatgcagagacagtggcagagacagagcgagagagcctgtgatggagagagacagggaatgcaattttaggcgaggaatccttggggaagggaagttgttgaagggaactcgcagactctgggggcacacccactttctccttggatcttgacacttgcatcttgtaaataacgtaattatcaccgccaccgccttcccccattttgtagctatggacaccaagtctcagagaagtgaagtgacttgcccaaggtcacgcagctggcgagtggcgcacaggggagggggacagctgaaataatcacagtgggcttatttttaatttttatttgtattttggtcgtggtgatgtgggtggaggtggagatggcaagttgggaaaagtaaaaacttccccttcctgcacggttcccagcaagggtgggggcctcctgtcttgcactttgcaaagttcaagaaatcccctttccctacccttcacgctgcacagccggccctctttccagacagtgcgatgccaataaaatgggaagtggggtgggagatgtcaagtcagatccaccacagccccgacacggggaggaagaggttaaagcctttgcggccggaaccgactcagggaagacgttctcaagcatcccgcacagacactgcctgctcgaccccctttctctagggatccggagcgtctgcgaccgcctggggccggggctgagactcccgtccctgtgcgcacctgttccgtgcgcccttgtgcggtgcgcacctgttccgtgcacccttgtcccgagcgccccagctccttgcgctcccgccgggggtgcgccctgcagggggcgcggcgagggggccgcgagggaccctccccaactccaccccttcggcctcctcccctttcccagccgcgggcagctccgggtctataaagagaggcgtccgaggacgcgcagggagatttggacgctccggcctgggaggtgcgtcagatccgagctcgccatccagtttcctctccactagtccccccagttggagatctgtaagtagtagttgtcattctgggggcagattgcagggcaggggggtgttaaaagtcctatagggtattctataggggctggggtgcacttaggggtctctggctaactagagaacccactgcttactggcttatcgaaattaatacgactcactatagggagacccaagctggctagcgccaccatggagcgcgacggctgcgcggggggcgggagccgcggcggcgagggcgggcgcgctccccgggagggcccggcggggaacggccgcgatcggggccgcagccacgctgccgaggcgcccggggacccgcaggccgcggcgtccttgctggcccctatggacgtgggggaggagccgctggagaaggcggcgcgcgcccgcactgccaaggaccccaacacctataaagtactctcgctggtattgtcagtatgtgtgttaacaacaatacttggttgtatatttgggttgaaaccaagctgtgccaaagaagttaaaagttgcaaaggtcgctgtttcgagagaacatttgggaactgtcgctgtgatgctgcctgtgttgagcttggaaactgctgtttagattaccaggagacgtgcatagaaccagaacatatatggacttgcaacaaattcaggtgtggtgagaaaaggttgaccagaagcctctgtgcctgttcagatgactgcaaggacaagggcgactgctgcatcaactacagttctgtgtgtcaaggtgagaaaagttgggtagaagaaccatgtgagagcattaatgagccacagtgcccagcagggtttgaaacgcctcctaccctcttattttctttggatggattcagggcagaatatttacacacttggggtggacttcttcctgttattagcaaactaaaaaaatgtggaacatatactaaaaacatgagaccggtatatccaacaaaaactttccccaatcactacagcattgtcaccggattgtatccagaatctcatggcataatcgacaataaaatgtatgatcccaaaatgaatgcttccttttcacttaaaagtaaagagaaatttaatcctgagtggtacaaaggagaaccaatttgggtcacagctaagtatcaaggcctcaagtctggcacatttttctggccaggatcagatgtggaaattaacggaattttcccagacatctataaaatgtataatggttcagtaccatttgaagaaaggattttagctgttcttcagtggctacagcttcctaaagatgaaagaccacacttttacactctgtatttagaagaaccagattcttcaggtcattcatatggaccagtcagcagtgaagtcatcaaagccttgcagagggttgatggtatggttggtatgctgatggatggtctgaaagagctgaacttgcacagatgcctgaacctcatccttatttcagatcatggcatggaacaaggcagttgtaagaaatacatatatctgaataaatatttgggggatgttaaaaatattaaagttatctatggacctgcagctcgattgagaccctctgatgtcccagataaatactattcatttaactatgaaggcattgcccgaaatctttcttgccgggaaccaaaccagcacttcaaaccttacctgaaacatttcttacctaagcgtttgcactttgctaagagtgatagaattgagcccttgacattctatttggaccctcagtggcaacttgcattgaatccctcagaaaggaaatattgtggaagtggatttcatggctctgacaatgtattttcaaatatgcaagccctctttgttggctatggacctggattcaagcatggcattgaggctgacacctttgaaaacattgaagtctataacttaatgtgtgatttactgaatttgacaccggctcctaataacggaactcatggaagtcttaaccaccttctaaagaatcctgtttatacgccaaagcatcccaaagaagtgcaccccctggtacagtgccccttcacaagaaaccccagagataaccttggctgctcatgtaacccttcgattttgccgattgaggattttcaaacacagttcaatctgactgtggcagaagagaagattattaagcatgaaactttaccctatggaagacctagagttctccagaaggaaaacaccatctgtcttctttcccagcaccagtttatgagtggatacagccaagacatcttaatgcccctttggacatcctataccgtggacagaaatgacagtttctctacggaagacttctccaactgtctgtaccaggactttagaattcctcttagtcctgtccataaatgttcattttataaaaataacaccaaagtgagttacgggttcctctccccaccacaactaaataaaaattcaagtggaatatattctgaagctttgcttactacaaatatagtgccaatgtaccagagttttcaagttatatggcgctactttcatgacaccctactgcgaaagtatgctgaagaaagaaatggtgtcaatgtcgtcagtggtcctgtgtttgactttgattatgatggacgttgtgattccttagagaatctgaggcaaaaaagaagagtcatccgtaaccaagaaattttgattccaactcacttctttattgtgctaacaagctgtaaagatacatctcagacgcctttgcactgtgaaaacctagacaccttagctttcattttgcctcacaggactgataacagcgagagctgtgtgcatgggaagcatgactcctcatgggttgaagaattgttaatgttacacagagcacggatcacagatgttgagcacatcactggactcagcttctatcaacaaagaaaagagccagtttcagacattttaaagttgaaaacacatttgccaacctttagccaagaagacgactacaaagacgatgacgacaagtgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 85 - DNA, 9702 bp, synthetic construct
  source 1..9702: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttaagattatagttgtcatttgaacttggggataaaggagacatctatgacttggctggaaaagacagagctaatgtacattgcaaagcacatatttatagcaggaaaatgggaagatttctctttaattctggagatggagtggggatggggagagtagactactcattttaagggtgaaacattggaattcaacttgtttgatgttatattaattggtggttaattactaagctaagtacgtataaaacttttatctatggctagcttgtccccccaaagtcatgcaatatagtgaactggctttcgcactttaaattattcattgatcatgtaatgattcagatgattcatcttccaagatggacactgaaactaacactcatagtaggttgtggtttaaagagtggaacaaccgccagtctcattagtggaaattgtgatggttgaatttatcaaggatgaacatacacggtcttctttctgagattttctttaagattttcgcacagataatctatttcttaggttttggagagaaaacttgaattttattgatccctcagaactcaatctttcagatttcaaaggagctatttcttttaatggggactctgttaatatttataaaagctcttcacaggatggagggtgggagggaaactccatcccaacaagacaaaaagaatgaagcatgaggctccacctagttcatcactgctccttgaaatacatcagtattgaaagacacatccaccccacccccaacccagccctattgctgttccagctcaagagtcagaggtcccgaagctgtagctcttctacaatctgctgctctgtgacttcaagtctgttgtctgcaaagaaaactattgggttcccaagcaagagaggcacatctggtaggacagattttgtgattgcaaaagaagggggaaaaaaagaaagaaagaaaagacctctctatacaagataaccagaggcatcaaactgaaatcctcctgtggaaaataagctagtacttctgggcctgatggtgtagtgaaaacctgtgcttgaggatacattacagtgaaagagcaaagtgaatagtaagtagctattacttacctccttagggaggtgtgttgtttgtctgtacatcccccacagcacctagcacagtaccttgcatctcacctgccactcactaaaaagtctatcaagttagttaattatcgagacaacgccctcagaaatgagagaacagtaccctcttatccttgctgcactttccagcactgatacgctgcctaaaagaggactagggcacaggtttgaattaatgtcacaaaactggatgggcaagttacaacggtgttgattaaggaaacagaactcatggtgcaccggatatctccatcctgatgaacccttggaaaaatgccaaagatgcatatccccaggcaaatgcctgattagtctgggattgatagattggtctaggattcagccctactgggaagatgtctaaattataatcagtgtagaaagcgaagttctcctagaagaagaggcaaaggttaaaaagaagaaaagaaaagaaagtgaagtcctttctcccccaaaacctctcatcaatcaatcagggtaacaaacagaacactagggctctgtctgtggaccaaacccaaaagccctgcggtcagggccaggagggtagatcatgtgtttgtggcaacttcctctgtgggcttttgcccaggtctgtccccaagcatacgatggccaaaacttctgcaccagagcagcatcctgtgtaacacagtcaggtccagcagttagggaaaactgcccactcagagtagataatatctggaaggaatgactgtttgggaaaagttccaatgctagttcagtgccaacccttccccaccttctccagctctctcccactggttcctcccctctcaactgctctggttcttataaaaacctcacagccttccactaacatcccgtaggagcctctctccctactgctgctacacaagaccctgagactgacctgcaggacgaaaccctctggctaactagagaacccactgcttactggcttatcgaaattaatacgactcactatagggagacccaagctggctagcgccaccatggagcgcgacggctgcgcggggggcgggagccgcggcggcgagggcgggcgcgctccccgggagggcccggcggggaacggccgcgatcggggccgcagccacgctgccgaggcgcccggggacccgcaggccgcggcgtccttgctggcccctatggacgtgggggaggagccgctggagaaggcggcgcgcgcccgcactgccaaggaccccaacacctataaagtactctcgctggtattgtcagtatgtgtgttaacaacaatacttggttgtatatttgggttgaaaccaagctgtgccaaagaagttaaaagttgcaaaggtcgctgtttcgagagaacatttgggaactgtcgctgtgatgctgcctgtgttgagcttggaaactgctgtttagattaccaggagacgtgcatagaaccagaacatatatggacttgcaacaaattcaggtgtggtgagaaaaggttgaccagaagcctctgtgcctgttcagatgactgcaaggacaagggcgactgctgcatcaactacagttctgtgtgtcaaggtgagaaaagttgggtagaagaaccatgtgagagcattaatgagccacagtgcccagcagggtttgaaacgcctcctaccctcttattttctttggatggattcagggcagaatatttacacacttggggtggacttcttcctgttattagcaaactaaaaaaatgtggaacatatactaaaaacatgagaccggtatatccaacaaaaactttccccaatcactacagcattgtcaccggattgtatccagaatctcatggcataatcgacaataaaatgtatgatcccaaaatgaatgcttccttttcacttaaaagtaaagagaaatttaatcctgagtggtacaaaggagaaccaatttgggtcacagctaagtatcaaggcctcaagtctggcacatttttctggccaggatcagatgtggaaattaacggaattttcccagacatctataaaatgtataatggttcagtaccatttgaagaaaggattttagctgttcttcagtggctacagcttcctaaagatgaaagaccacacttttacactctgtatttagaagaaccagattcttcaggtcattcatatggaccagtcagcagtgaagtcatcaaagccttgcagagggttgatggtatggttggtatgctgatggatggtctgaaagagctgaacttgcacagatgcctgaacctcatccttatttcagatcatggcatggaacaaggcagttgtaagaaatacatatatctgaataaatatttgggggatgttaaaaatattaaagttatctatggacctgcagctcgattgagaccctctgatgtcccagataaatactattcatttaactatgaaggcattgcccgaaatctttcttgccgggaaccaaaccagcacttcaaaccttacctgaaacatttcttacctaagcgtttgcactttgctaagagtgatagaattgagcccttgacattctatttggaccctcagtggcaacttgcattgaatccctcagaaaggaaatattgtggaagtggatttcatggctctgacaatgtattttcaaatatgcaagccctctttgttggctatggacctggattcaagcatggcattgaggctgacacctttgaaaacattgaagtctataacttaatgtgtgatttactgaatttgacaccggctcctaataacggaactcatggaagtcttaaccaccttctaaagaatcctgtttatacgccaaagcatcccaaagaagtgcaccccctggtacagtgccccttcacaagaaaccccagagataaccttggctgctcatgtaacccttcgattttgccgattgaggattttcaaacacagttcaatctgactgtggcagaagagaagattattaagcatgaaactttaccctatggaagacctagagttctccagaaggaaaacaccatctgtcttctttcccagcaccagtttatgagtggatacagccaagacatcttaatgcccctttggacatcctataccgtggacagaaatgacagtttctctacggaagacttctccaactgtctgtaccaggactttagaattcctcttagtcctgtccataaatgttcattttataaaaataacaccaaagtgagttacgggttcctctccccaccacaactaaataaaaattcaagtggaatatattctgaagctttgcttactacaaatatagtgccaatgtaccagagttttcaagttatatggcgctactttcatgacaccctactgcgaaagtatgctgaagaaagaaatggtgtcaatgtcgtcagtggtcctgtgtttgactttgattatgatggacgttgtgattccttagagaatctgaggcaaaaaagaagagtcatccgtaaccaagaaattttgattccaactcacttctttattgtgctaacaagctgtaaagatacatctcagacgcctttgcactgtgaaaacctagacaccttagctttcattttgcctcacaggactgataacagcgagagctgtgtgcatgggaagcatgactcctcatgggttgaagaattgttaatgttacacagagcacggatcacagatgttgagcacatcactggactcagcttctatcaacaaagaaaagagccagtttcagacattttaaagttgaaaacacatttgccaacctttagccaagaagacgactacaaagacgatgacgacaagtgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 86 - DNA, 7856 bp, synthetic construct
  source 1..7856: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgaaaactacaattgactatctctaaattttaaaatgtgatgatagttactacttcagtggtccacactgtttctatcctttggaacataagattttgttgtgtagcttaaggaagaggaattcagcagttgactatctcgattgttctgggaattttggaggctgaggggaaattttcacatataagcgctgggtttgctggggtattgagagtgacctctggctaactagagaacccactgcttactggcttatcgaaattaatacgactcactatagggagacccaagctggctagcgccaccatggagcgcgacggctgcgcggggggcgggagccgcggcggcgagggcgggcgcgctccccgggagggcccggcggggaacggccgcgatcggggccgcagccacgctgccgaggcgcccggggacccgcaggccgcggcgtccttgctggcccctatggacgtgggggaggagccgctggagaaggcggcgcgcgcccgcactgccaaggaccccaacacctataaagtactctcgctggtattgtcagtatgtgtgttaacaacaatacttggttgtatatttgggttgaaaccaagctgtgccaaagaagttaaaagttgcaaaggtcgctgtttcgagagaacatttgggaactgtcgctgtgatgctgcctgtgttgagcttggaaactgctgtttagattaccaggagacgtgcatagaaccagaacatatatggacttgcaacaaattcaggtgtggtgagaaaaggttgaccagaagcctctgtgcctgttcagatgactgcaaggacaagggcgactgctgcatcaactacagttctgtgtgtcaaggtgagaaaagttgggtagaagaaccatgtgagagcattaatgagccacagtgcccagcagggtttgaaacgcctcctaccctcttattttctttggatggattcagggcagaatatttacacacttggggtggacttcttcctgttattagcaaactaaaaaaatgtggaacatatactaaaaacatgagaccggtatatccaacaaaaactttccccaatcactacagcattgtcaccggattgtatccagaatctcatggcataatcgacaataaaatgtatgatcccaaaatgaatgcttccttttcacttaaaagtaaagagaaatttaatcctgagtggtacaaaggagaaccaatttgggtcacagctaagtatcaaggcctcaagtctggcacatttttctggccaggatcagatgtggaaattaacggaattttcccagacatctataaaatgtataatggttcagtaccatttgaagaaaggattttagctgttcttcagtggctacagcttcctaaagatgaaagaccacacttttacactctgtatttagaagaaccagattcttcaggtcattcatatggaccagtcagcagtgaagtcatcaaagccttgcagagggttgatggtatggttggtatgctgatggatggtctgaaagagctgaacttgcacagatgcctgaacctcatccttatttcagatcatggcatggaacaaggcagttgtaagaaatacatatatctgaataaatatttgggggatgttaaaaatattaaagttatctatggacctgcagctcgattgagaccctctgatgtcccagataaatactattcatttaactatgaaggcattgcccgaaatctttcttgccgggaaccaaaccagcacttcaaaccttacctgaaacatttcttacctaagcgtttgcactttgctaagagtgatagaattgagcccttgacattctatttggaccctcagtggcaacttgcattgaatccctcagaaaggaaatattgtggaagtggatttcatggctctgacaatgtattttcaaatatgcaagccctctttgttggctatggacctggattcaagcatggcattgaggctgacacctttgaaaacattgaagtctataacttaatgtgtgatttactgaatttgacaccggctcctaataacggaactcatggaagtcttaaccaccttctaaagaatcctgtttatacgccaaagcatcccaaagaagtgcaccccctggtacagtgccccttcacaagaaaccccagagataaccttggctgctcatgtaacccttcgattttgccgattgaggattttcaaacacagttcaatctgactgtggcagaagagaagattattaagcatgaaactttaccctatggaagacctagagttctccagaaggaaaacaccatctgtcttctttcccagcaccagtttatgagtggatacagccaagacatcttaatgcccctttggacatcctataccgtggacagaaatgacagtttctctacggaagacttctccaactgtctgtaccaggactttagaattcctcttagtcctgtccataaatgttcattttataaaaataacaccaaagtgagttacgggttcctctccccaccacaactaaataaaaattcaagtggaatatattctgaagctttgcttactacaaatatagtgccaatgtaccagagttttcaagttatatggcgctactttcatgacaccctactgcgaaagtatgctgaagaaagaaatggtgtcaatgtcgtcagtggtcctgtgtttgactttgattatgatggacgttgtgattccttagagaatctgaggcaaaaaagaagagtcatccgtaaccaagaaattttgattccaactcacttctttattgtgctaacaagctgtaaagatacatctcagacgcctttgcactgtgaaaacctagacaccttagctttcattttgcctcacaggactgataacagcgagagctgtgtgcatgggaagcatgactcctcatgggttgaagaattgttaatgttacacagagcacggatcacagatgttgagcacatcactggactcagcttctatcaacaaagaaaagagccagtttcagacattttaaagttgaaaacacatttgccaacctttagccaagaagacgactacaaagacgatgacgacaagtgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 87 - DNA, 8989 bp, synthetic construct
  source 1..8989: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttataaatgttttgtagaataaaaaaaaaaaaagttcttcaaaagaaatctcaaatctccaaatggaaacaggtaaaagtggagctcccctggttccacggagaaccttttttgaggaaacttaggcaactcgcaggtaccttatgtcatgagacagagtttgaaaactacaattgactatctctaaatttcctcccaggtctaaaatgtgatgatagttactacttcagtacatcatccttaaggaaaattattaggtccacactgtttctatcctttgaattttacacataaattttgtaatcaaaagtttatttgtaatatcagatggaatcagataattgctttttgttttttccactgacaggaacataagattttgttgtgtagcttaagtcaaacgcagtttggaatatatattttttaaaaattgtaacttacatatccaaatacaatttttcaagaagtagagtattcagtagaaattaatctgtgaaagaagaggaattcagcagtggcctatttgatgaatgatttaacgtgcttatttcttccctttcatcaaaactctgtgtccccttgtttgccccctctgacttcatactctggagttgaccaagatccctcttccatcggattgttctgggaattttgaaataatctgctttttcctctctttcccctgttgcttctgatgccttagaattacattttcctcgctgatttagtttagaaaagagaaaagagcttccatgactagtagattatcacttttgggtttgctcttggaagtgacaagatgctaggatccctctttggaatgtaaaatttatctcttatatagaaaggatataaatgtagcaccagagactataaaactctgatactatctactgtactgtatagctgaacgccacaatgtgtctggtaatctattgactatcataaatgctatttctacagaaaagttaggaggtccatatttcgggcaaccaatgtatagctgaatgcagaacagtcatagttgggtactaaccatatatatgatttatccatcaacaggtgcatatgctcagaaattctgtatccataagaaatcagactactttcttttccttttgcaagtaaattgaatttagcctgagaggctgaggggaaattttcacatataagccacggttttgtgttttgtgttttgttttgtttatagatatagtactaactggatggatgcgataaaattcataggtggtactaagatacaataggatttgtgaaatggacaattgtcttgcataaatagcaagtaaaaaatcaagcctgtccttcataaaaattttattctggggtgtctctggctaactagagaacccactgcttactggcttatcgaaattaatacgactcactatagggagacccaagctggctagcgccaccatggagcgcgacggctgcgcggggggcgggagccgcggcggcgagggcgggcgcgctccccgggagggcccggcggggaacggccgcgatcggggccgcagccacgctgccgaggcgcccggggacccgcaggccgcggcgtccttgctggcccctatggacgtgggggaggagccgctggagaaggcggcgcgcgcccgcactgccaaggaccccaacacctataaagtactctcgctggtattgtcagtatgtgtgttaacaacaatacttggttgtatatttgggttgaaaccaagctgtgccaaagaagttaaaagttgcaaaggtcgctgtttcgagagaacatttgggaactgtcgctgtgatgctgcctgtgttgagcttggaaactgctgtttagattaccaggagacgtgcatagaaccagaacatatatggacttgcaacaaattcaggtgtggtgagaaaaggttgaccagaagcctctgtgcctgttcagatgactgcaaggacaagggcgactgctgcatcaactacagttctgtgtgtcaaggtgagaaaagttgggtagaagaaccatgtgagagcattaatgagccacagtgcccagcagggtttgaaacgcctcctaccctcttattttctttggatggattcagggcagaatatttacacacttggggtggacttcttcctgttattagcaaactaaaaaaatgtggaacatatactaaaaacatgagaccggtatatccaacaaaaactttccccaatcactacagcattgtcaccggattgtatccagaatctcatggcataatcgacaataaaatgtatgatcccaaaatgaatgcttccttttcacttaaaagtaaagagaaatttaatcctgagtggtacaaaggagaaccaatttgggtcacagctaagtatcaaggcctcaagtctggcacatttttctggccaggatcagatgtggaaattaacggaattttcccagacatctataaaatgtataatggttcagtaccatttgaagaaaggattttagctgttcttcagtggctacagcttcctaaagatgaaagaccacacttttacactctgtatttagaagaaccagattcttcaggtcattcatatggaccagtcagcagtgaagtcatcaaagccttgcagagggttgatggtatggttggtatgctgatggatggtctgaaagagctgaacttgcacagatgcctgaacctcatccttatttcagatcatggcatggaacaaggcagttgtaagaaatacatatatctgaataaatatttgggggatgttaaaaatattaaagttatctatggacctgcagctcgattgagaccctctgatgtcccagataaatactattcatttaactatgaaggcattgcccgaaatctttcttgccgggaaccaaaccagcacttcaaaccttacctgaaacatttcttacctaagcgtttgcactttgctaagagtgatagaattgagcccttgacattctatttggaccctcagtggcaacttgcattgaatccctcagaaaggaaatattgtggaagtggatttcatggctctgacaatgtattttcaaatatgcaagccctctttgttggctatggacctggattcaagcatggcattgaggctgacacctttgaaaacattgaagtctataacttaatgtgtgatttactgaatttgacaccggctcctaataacggaactcatggaagtcttaaccaccttctaaagaatcctgtttatacgccaaagcatcccaaagaagtgcaccccctggtacagtgccccttcacaagaaaccccagagataaccttggctgctcatgtaacccttcgattttgccgattgaggattttcaaacacagttcaatctgactgtggcagaagagaagattattaagcatgaaactttaccctatggaagacctagagttctccagaaggaaaacaccatctgtcttctttcccagcaccagtttatgagtggatacagccaagacatcttaatgcccctttggacatcctataccgtggacagaaatgacagtttctctacggaagacttctccaactgtctgtaccaggactttagaattcctcttagtcctgtccataaatgttcattttataaaaataacaccaaagtgagttacgggttcctctccccaccacaactaaataaaaattcaagtggaatatattctgaagctttgcttactacaaatatagtgccaatgtaccagagttttcaagttatatggcgctactttcatgacaccctactgcgaaagtatgctgaagaaagaaatggtgtcaatgtcgtcagtggtcctgtgtttgactttgattatgatggacgttgtgattccttagagaatctgaggcaaaaaagaagagtcatccgtaaccaagaaattttgattccaactcacttctttattgtgctaacaagctgtaaagatacatctcagacgcctttgcactgtgaaaacctagacaccttagctttcattttgcctcacaggactgataacagcgagagctgtgtgcatgggaagcatgactcctcatgggttgaagaattgttaatgttacacagagcacggatcacagatgttgagcacatcactggactcagcttctatcaacaaagaaaagagccagtttcagacattttaaagttgaaaacacatttgccaacctttagccaagaagacgactacaaagacgatgacgacaagtgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 88 - DNA, 9409 bp, synthetic construct
  source 1..9409: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttggcacctctggctaactagagaacccactgcttactggcttatcgaaattaatacgactcactatagggagacccaagctggctagcgccaccatgaagtgggtaacctttatttcccttctttttctctttagctcggcttattccaggggtgtgtttcgtcgagactacaaagacgatgacgacaagagcgctggcctgaagccaagctgcgccaaggaggtgaagagctgcaagggcagatgtttcgaacggaccttcggcaactgcagatgcgacgccgcctgcgtggaactgggaaactgttgtctggactaccaggagacatgtatcgagcctgagcacatctggacatgcaacaagttcagatgtggcgagaaaagactgactcggagtctgtgcgcctgttctgacgactgcaaggacaagggcgattgctgcatcaactacagcagcgtgtgccagggcgaaaagagctgggtcgaggaaccctgcgagagcatcaacgagccccagtgccctgccggatttgagacacctcctaccctgctcttctccctggatggcttcagagctgagtacctgcatacatggggaggactgctgcctgtgatcagcaagctgaagaagtgcggcacctacacaaagaacatgcggcctgtctaccctaccaagaccttccccaaccactacagcatcgtgaccggcctgtaccccgagagccacggcatcatcgacaacaagatgtacgaccccaaaatgaacgccagcttcagcctgaaatctaaggaaaagttcaaccctgagtggtataagggcgagccaatctgggtgacagccaagtaccagggcctgaagagcggcaccttcttctggcctggcagcgacgtggagatcaacggaatcttccctgatatctacaagatgtataacggctccgtgcctttcgaggagcggatcttggctgttcttcagtggctgcagctgcccaaggacgagagaccccacttctacaccctgtacctggaagaacctgacagcagcggccacagctacggccctgtgtctagcgaggtgatcaaggccctgcaaagagtggatggcatggtgggcatgctgatggatggactgaaggaactgaacctccatcgttgcctgaacctgatcctgatctccgaccacggcatggaacagggcagctgtaaaaagtacatctacctgaacaagtacctgggcgacgtgaagaatatcaaggtgatctacgggccggccgccaggctgcggcctagcgacgtgcccgacaagtattacagcttcaactacgagggcatcgccagaaacctgtcttgcagagaacccaatcaacacttcaagccttacctgaagcactttctgcctaagagactgcacttcgccaaaagcgacagaattgaacccctgacattctatctggaccctcagtggcagctggccctcaatccttctgaacgcaaatactgcggtagcggatttcacggctctgataacgtgttttctaacatgcaggccctgtttgtgggctacggccctggcttcaagcacggaatcgaggctgacaccttcgagaacatcgaagtttacaatctgatgtgcgacctgctgaatctgaccccagcccctaacaacggcacacacggctctctgaatcacctgctcaagaaccccgtgtatacccccaaacaccctaaagaggtccatccactggtgcagtgcccattcaccagaaaccctcgggacaaccttggctgcagctgcaaccctagcatcctccctatcgaagatttccagacccagtttaacctgactgtcgccgaagagaagatcatcaagcacgaaaccctgccctacgggaggcccagagtgctgcagaaagaaaacacaatctgtctgctgagccagcaccagtttatgagcggctacagtcaggacatcctgatgcctctgtggaccagctacaccgtggaccggaatgacagcttcagcaccgaggacttcagcaactgcctgtatcaggacttccgcatccctctgtcccctgtgcataagtgcagcttttacaagaacaacaccaaggtgagctacgggtttctgagcccaccacaactgaacaaaaatagctctggcatctactctgaggcgctgctgaccaccaacatcgtgccaatgtaccagagcttccaggtgatctggcggtacttccatgataccctgcttcggaagtacgccgaagaacggaacggcgtgaacgtggtgtccggcccagtgttcgacttcgactacgacggcagatgtgattcccttgagaatctgagacagaagcggcgggttatcagaaaccaggagatcctgattcctacccacttcttcatcgtgctgacatcttgcaaggataccagccaaacacctctgcactgcgagaacctggacaccctggccttcatcctgcctcaccggaccgataatagcgaatcctgtgtgcacggcaaacacgactctagctgggtggaagagctgctgatgctgcacagagcccggatcacagatgtggaacacatcaccggcctgtccttctaccaacagagaaaggaacccgtgtctgatatcctgaagctgaaaacccacctgcctaccttcagccaggaggacctgatcgttaacgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccacactcgagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 89 - DNA, 9843 bp, synthetic construct
  source 1..9843: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatgaagtgggtaacctttatttcccttctttttctctttagctcggcttattccaggggtgtgtttcgtcgagactacaaagacgatgacgacaagagcgctggcctgaagccaagctgcgccaaggaggtgaagagctgcaagggcagatgtttcgaacggaccttcggcaactgcagatgcgacgccgcctgcgtggaactgggaaactgttgtctggactaccaggagacatgtatcgagcctgagcacatctggacatgcaacaagttcagatgtggcgagaaaagactgactcggagtctgtgcgcctgttctgacgactgcaaggacaagggcgattgctgcatcaactacagcagcgtgtgccagggcgaaaagagctgggtcgaggaaccctgcgagagcatcaacgagccccagtgccctgccggatttgagacacctcctaccctgctcttctccctggatggcttcagagctgagtacctgcatacatggggaggactgctgcctgtgatcagcaagctgaagaagtgcggcacctacacaaagaacatgcggcctgtctaccctaccaagaccttccccaaccactacagcatcgtgaccggcctgtaccccgagagccacggcatcatcgacaacaagatgtacgaccccaaaatgaacgccagcttcagcctgaaatctaaggaaaagttcaaccctgagtggtataagggcgagccaatctgggtgacagccaagtaccagggcctgaagagcggcaccttcttctggcctggcagcgacgtggagatcaacggaatcttccctgatatctacaagatgtataacggctccgtgcctttcgaggagcggatcttggctgttcttcagtggctgcagctgcccaaggacgagagaccccacttctacaccctgtacctggaagaacctgacagcagcggccacagctacggccctgtgtctagcgaggtgatcaaggccctgcaaagagtggatggcatggtgggcatgctgatggatggactgaaggaactgaacctccatcgttgcctgaacctgatcctgatctccgaccacggcatggaacagggcagctgtaaaaagtacatctacctgaacaagtacctgggcgacgtgaagaatatcaaggtgatctacgggccggccgccaggctgcggcctagcgacgtgcccgacaagtattacagcttcaactacgagggcatcgccagaaacctgtcttgcagagaacccaatcaacacttcaagccttacctgaagcactttctgcctaagagactgcacttcgccaaaagcgacagaattgaacccctgacattctatctggaccctcagtggcagctggccctcaatccttctgaacgcaaatactgcggtagcggatttcacggctctgataacgtgttttctaacatgcaggccctgtttgtgggctacggccctggcttcaagcacggaatcgaggctgacaccttcgagaacatcgaagtttacaatctgatgtgcgacctgctgaatctgaccccagcccctaacaacggcacacacggctctctgaatcacctgctcaagaaccccgtgtatacccccaaacaccctaaagaggtccatccactggtgcagtgcccattcaccagaaaccctcgggacaaccttggctgcagctgcaaccctagcatcctccctatcgaagatttccagacccagtttaacctgactgtcgccgaagagaagatcatcaagcacgaaaccctgccctacgggaggcccagagtgctgcagaaagaaaacacaatctgtctgctgagccagcaccagtttatgagcggctacagtcaggacatcctgatgcctctgtggaccagctacaccgtggaccggaatgacagcttcagcaccgaggacttcagcaactgcctgtatcaggacttccgcatccctctgtcccctgtgcataagtgcagcttttacaagaacaacaccaaggtgagctacgggtttctgagcccaccacaactgaacaaaaatagctctggcatctactctgaggcgctgctgaccaccaacatcgtgccaatgtaccagagcttccaggtgatctggcggtacttccatgataccctgcttcggaagtacgccgaagaacggaacggcgtgaacgtggtgtccggcccagtgttcgacttcgactacgacggcagatgtgattcccttgagaatctgagacagaagcggcgggttatcagaaaccaggagatcctgattcctacccacttcttcatcgtgctgacatcttgcaaggataccagccaaacacctctgcactgcgagaacctggacaccctggccttcatcctgcctcaccggaccgataatagcgaatcctgtgtgcacggcaaacacgactctagctgggtggaagagctgctgatgctgcacagagcccggatcacagatgtggaacacatcaccggcctgtccttctaccaacagagaaaggaacccgtgtctgatatcctgaagctgaaaacccacctgcctaccttcagccaggaggacctgatcgttaacgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccacactcgagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatgacaattacgattagcactatccaattacgattagcactatccaattacgattagcactatcggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 90 - DNA, 10549 bp, synthetic construct
  source 1..10549: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttataaatgttttgtagaataaaaaaaaaaaaagttcttcaaaagaaatctcaaatctccaaatggaaacaggtaaaagtggagctcccctggttccacggagaaccttttttgaggaaacttaggcaactcgcaggtaccttatgtcatgagacagagtttgaaaactacaattgactatctctaaatttcctcccaggtctaaaatgtgatgatagttactacttcagtacatcatccttaaggaaaattattaggtccacactgtttctatcctttgaattttacacataaattttgtaatcaaaagtttatttgtaatatcagatggaatcagataattgctttttgttttttccactgacaggaacataagattttgttgtgtagcttaagtcaaacgcagtttggaatatatattttttaaaaattgtaacttacatatccaaatacaatttttcaagaagtagagtattcagtagaaattaatctgtgaaagaagaggaattcagcagtggcctatttgatgaatgatttaacgtgcttatttcttccctttcatcaaaactctgtgtccccttgtttgccccctctgacttcatactctggagttgaccaagatccctcttccatcggattgttctgggaattttgaaataatctgctttttcctctctttcccctgttgcttctgatgccttagaattacattttcctcgctgatttagtttagaaaagagaaaagagcttccatgactagtagattatcacttttgggtttgctcttggaagtgacaagatgctaggatccctctttggaatgtaaaatttatctcttatatagaaaggatataaatgtagcaccagagactataaaactctgatactatctactgtactgtatagctgaacgccacaatgtgtctggtaatctattgactatcataaatgctatttctacagaaaagttaggaggtccatatttcgggcaaccaatgtatagctgaatgcagaacagtcatagttgggtactaaccatatatatgatttatccatcaacaggtgcatatgctcagaaattctgtatccataagaaatcagactactttcttttccttttgcaagtaaattgaatttagcctgagaggctgaggggaaattttcacatataagccacggttttgtgttttgtgttttgttttgtttatagatatagtactaactggatggatgcgataaaattcataggtggtactaagatacaataggatttgtgaaatggacaattgtcttgcataaatagcaagtaaaaaatcaagcctgtccttcataaaaattttattctggggtgtctctggctaactagagaacccactgcttactggcttatcgaaattaatacgactcactatagggagacccaagctggctagcgccaccatgaagtgggtaacctttatttcccttctttttctctttagctcggcttattccaggggtgtgtttcgtcgagactacaaagacgatgacgacaagagcgctggcctgaagccaagctgcgccaaggaggtgaagagctgcaagggcagatgtttcgaacggaccttcggcaactgcagatgcgacgccgcctgcgtggaactgggaaactgttgtctggactaccaggagacatgtatcgagcctgagcacatctggacatgcaacaagttcagatgtggcgagaaaagactgactcggagtctgtgcgcctgttctgacgactgcaaggacaagggcgattgctgcatcaactacagcagcgtgtgccagggcgaaaagagctgggtcgaggaaccctgcgagagcatcaacgagccccagtgccctgccggatttgagacacctcctaccctgctcttctccctggatggcttcagagctgagtacctgcatacatggggaggactgctgcctgtgatcagcaagctgaagaagtgcggcacctacacaaagaacatgcggcctgtctaccctaccaagaccttccccaaccactacagcatcgtgaccggcctgtaccccgagagccacggcatcatcgacaacaagatgtacgaccccaaaatgaacgccagcttcagcctgaaatctaaggaaaagttcaaccctgagtggtataagggcgagccaatctgggtgacagccaagtaccagggcctgaagagcggcaccttcttctggcctggcagcgacgtggagatcaacggaatcttccctgatatctacaagatgtataacggctccgtgcctttcgaggagcggatcttggctgttcttcagtggctgcagctgcccaaggacgagagaccccacttctacaccctgtacctggaagaacctgacagcagcggccacagctacggccctgtgtctagcgaggtgatcaaggccctgcaaagagtggatggcatggtgggcatgctgatggatggactgaaggaactgaacctccatcgttgcctgaacctgatcctgatctccgaccacggcatggaacagggcagctgtaaaaagtacatctacctgaacaagtacctgggcgacgtgaagaatatcaaggtgatctacgggccggccgccaggctgcggcctagcgacgtgcccgacaagtattacagcttcaactacgagggcatcgccagaaacctgtcttgcagagaacccaatcaacacttcaagccttacctgaagcactttctgcctaagagactgcacttcgccaaaagcgacagaattgaacccctgacattctatctggaccctcagtggcagctggccctcaatccttctgaacgcaaatactgcggtagcggatttcacggctctgataacgtgttttctaacatgcaggccctgtttgtgggctacggccctggcttcaagcacggaatcgaggctgacaccttcgagaacatcgaagtttacaatctgatgtgcgacctgctgaatctgaccccagcccctaacaacggcacacacggctctctgaatcacctgctcaagaaccccgtgtatacccccaaacaccctaaagaggtccatccactggtgcagtgcccattcaccagaaaccctcgggacaaccttggctgcagctgcaaccctagcatcctccctatcgaagatttccagacccagtttaacctgactgtcgccgaagagaagatcatcaagcacgaaaccctgccctacgggaggcccagagtgctgcagaaagaaaacacaatctgtctgctgagccagcaccagtttatgagcggctacagtcaggacatcctgatgcctctgtggaccagctacaccgtggaccggaatgacagcttcagcaccgaggacttcagcaactgcctgtatcaggacttccgcatccctctgtcccctgtgcataagtgcagcttttacaagaacaacaccaaggtgagctacgggtttctgagcccaccacaactgaacaaaaatagctctggcatctactctgaggcgctgctgaccaccaacatcgtgccaatgtaccagagcttccaggtgatctggcggtacttccatgataccctgcttcggaagtacgccgaagaacggaacggcgtgaacgtggtgtccggcccagtgttcgacttcgactacgacggcagatgtgattcccttgagaatctgagacagaagcggcgggttatcagaaaccaggagatcctgattcctacccacttcttcatcgtgctgacatcttgcaaggataccagccaaacacctctgcactgcgagaacctggacaccctggccttcatcctgcctcaccggaccgataatagcgaatcctgtgtgcacggcaaacacgactctagctgggtggaagagctgctgatgctgcacagagcccggatcacagatgtggaacacatcaccggcctgtccttctaccaacagagaaaggaacccgtgtctgatatcctgaagctgaaaacccacctgcctaccttcagccaggaggacctgatcgttaacgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccacactcgagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 91 - DNA, 9783 bp, synthetic construct
  source 1..9783: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccaccatgaagtgggtaacctttatttcccttctttttctctttagctcggcttattccaggggtgtgtttcgtcgagactacaaagacgatgacgacaagagcgctggcctgaagccaagctgcgccaaggaggtgaagagctgcaagggcagatgtttcgaacggaccttcggcaactgcagatgcgacgccgcctgcgtggaactgggaaactgttgtctggactaccaggagacatgtatcgagcctgagcacatctggacatgcaacaagttcagatgtggcgagaaaagactgactcggagtctgtgcgcctgttctgacgactgcaaggacaagggcgattgctgcatcaactacagcagcgtgtgccagggcgaaaagagctgggtcgaggaaccctgcgagagcatcaacgagccccagtgccctgccggatttgagacacctcctaccctgctcttctccctggatggcttcagagctgagtacctgcatacatggggaggactgctgcctgtgatcagcaagctgaagaagtgcggcacctacacaaagaacatgcggcctgtctaccctaccaagaccttccccaaccactacagcatcgtgaccggcctgtaccccgagagccacggcatcatcgacaacaagatgtacgaccccaaaatgaacgccagcttcagcctgaaatctaaggaaaagttcaaccctgagtggtataagggcgagccaatctgggtgacagccaagtaccagggcctgaagagcggcaccttcttctggcctggcagcgacgtggagatcaacggaatcttccctgatatctacaagatgtataacggctccgtgcctttcgaggagcggatcttggctgttcttcagtggctgcagctgcccaaggacgagagaccccacttctacaccctgtacctggaagaacctgacagcagcggccacagctacggccctgtgtctagcgaggtgatcaaggccctgcaaagagtggatggcatggtgggcatgctgatggatggactgaaggaactgaacctccatcgttgcctgaacctgatcctgatctccgaccacggcatggaacagggcagctgtaaaaagtacatctacctgaacaagtacctgggcgacgtgaagaatatcaaggtgatctacgggccggccgccaggctgcggcctagcgacgtgcccgacaagtattacagcttcaactacgagggcatcgccagaaacctgtcttgcagagaacccaatcaacacttcaagccttacctgaagcactttctgcctaagagactgcacttcgccaaaagcgacagaattgaacccctgacattctatctggaccctcagtggcagctggccctcaatccttctgaacgcaaatactgcggtagcggatttcacggctctgataacgtgttttctaacatgcaggccctgtttgtgggctacggccctggcttcaagcacggaatcgaggctgacaccttcgagaacatcgaagtttacaatctgatgtgcgacctgctgaatctgaccccagcccctaacaacggcacacacggctctctgaatcacctgctcaagaaccccgtgtatacccccaaacaccctaaagaggtccatccactggtgcagtgcccattcaccagaaaccctcgggacaaccttggctgcagctgcaaccctagcatcctccctatcgaagatttccagacccagtttaacctgactgtcgccgaagagaagatcatcaagcacgaaaccctgccctacgggaggcccagagtgctgcagaaagaaaacacaatctgtctgctgagccagcaccagtttatgagcggctacagtcaggacatcctgatgcctctgtggaccagctacaccgtggaccggaatgacagcttcagcaccgaggacttcagcaactgcctgtatcaggacttccgcatccctctgtcccctgtgcataagtgcagcttttacaagaacaacaccaaggtgagctacgggtttctgagcccaccacaactgaacaaaaatagctctggcatctactctgaggcgctgctgaccaccaacatcgtgccaatgtaccagagcttccaggtgatctggcggtacttccatgataccctgcttcggaagtacgccgaagaacggaacggcgtgaacgtggtgtccggcccagtgttcgacttcgactacgacggcagatgtgattcccttgagaatctgagacagaagcggcgggttatcagaaaccaggagatcctgattcctacccacttcttcatcgtgctgacatcttgcaaggataccagccaaacacctctgcactgcgagaacctggacaccctggccttcatcctgcctcaccggaccgataatagcgaatcctgtgtgcacggcaaacacgactctagctgggtggaagagctgctgatgctgcacagagcccggatcacagatgtggaacacatcaccggcctgtccttctaccaacagagaaaggaacccgtgtctgatatcctgaagctgaaaacccacctgcctaccttcagccaggaggacctgatcgttaacgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccacactcgagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 92 - DNA, 9783 bp, synthetic construct
  source 1..9783: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgctctggctaactagagaacccactgcttactggcttatcgaaattaatacgactcactatagggagacccaagctggctagcgccaccatgaagtgggtaacctttatttcccttctttttctctttagctcggcttattccaggggtgtgtttcgtcgagactacaaagacgatgacgacaagagcgctggcctgaagccaagctgcgccaaggaggtgaagagctgcaagggcagatgtttcgaacggaccttcggcaactgcagatgcgacgccgcctgcgtggaactgggaaactgttgtctggactaccaggagacatgtatcgagcctgagcacatctggacatgcaacaagttcagatgtggcgagaaaagactgactcggagtctgtgcgcctgttctgacgactgcaaggacaagggcgattgctgcatcaactacagcagcgtgtgccagggcgaaaagagctgggtcgaggaaccctgcgagagcatcaacgagccccagtgccctgccggatttgagacacctcctaccctgctcttctccctggatggcttcagagctgagtacctgcatacatggggaggactgctgcctgtgatcagcaagctgaagaagtgcggcacctacacaaagaacatgcggcctgtctaccctaccaagaccttccccaaccactacagcatcgtgaccggcctgtaccccgagagccacggcatcatcgacaacaagatgtacgaccccaaaatgaacgccagcttcagcctgaaatctaaggaaaagttcaaccctgagtggtataagggcgagccaatctgggtgacagccaagtaccagggcctgaagagcggcaccttcttctggcctggcagcgacgtggagatcaacggaatcttccctgatatctacaagatgtataacggctccgtgcctttcgaggagcggatcttggctgttcttcagtggctgcagctgcccaaggacgagagaccccacttctacaccctgtacctggaagaacctgacagcagcggccacagctacggccctgtgtctagcgaggtgatcaaggccctgcaaagagtggatggcatggtgggcatgctgatggatggactgaaggaactgaacctccatcgttgcctgaacctgatcctgatctccgaccacggcatggaacagggcagctgtaaaaagtacatctacctgaacaagtacctgggcgacgtgaagaatatcaaggtgatctacgggccggccgccaggctgcggcctagcgacgtgcccgacaagtattacagcttcaactacgagggcatcgccagaaacctgtcttgcagagaacccaatcaacacttcaagccttacctgaagcactttctgcctaagagactgcacttcgccaaaagcgacagaattgaacccctgacattctatctggaccctcagtggcagctggccctcaatccttctgaacgcaaatactgcggtagcggatttcacggctctgataacgtgttttctaacatgcaggccctgtttgtgggctacggccctggcttcaagcacggaatcgaggctgacaccttcgagaacatcgaagtttacaatctgatgtgcgacctgctgaatctgaccccagcccctaacaacggcacacacggctctctgaatcacctgctcaagaaccccgtgtatacccccaaacaccctaaagaggtccatccactggtgcagtgcccattcaccagaaaccctcgggacaaccttggctgcagctgcaaccctagcatcctccctatcgaagatttccagacccagtttaacctgactgtcgccgaagagaagatcatcaagcacgaaaccctgccctacgggaggcccagagtgctgcagaaagaaaacacaatctgtctgctgagccagcaccagtttatgagcggctacagtcaggacatcctgatgcctctgtggaccagctacaccgtggaccggaatgacagcttcagcaccgaggacttcagcaactgcctgtatcaggacttccgcatccctctgtcccctgtgcataagtgcagcttttacaagaacaacaccaaggtgagctacgggtttctgagcccaccacaactgaacaaaaatagctctggcatctactctgaggcgctgctgaccaccaacatcgtgccaatgtaccagagcttccaggtgatctggcggtacttccatgataccctgcttcggaagtacgccgaagaacggaacggcgtgaacgtggtgtccggcccagtgttcgacttcgactacgacggcagatgtgattcccttgagaatctgagacagaagcggcgggttatcagaaaccaggagatcctgattcctacccacttcttcatcgtgctgacatcttgcaaggataccagccaaacacctctgcactgcgagaacctggacaccctggccttcatcctgcctcaccggaccgataatagcgaatcctgtgtgcacggcaaacacgactctagctgggtggaagagctgctgatgctgcacagagcccggatcacagatgtggaacacatcaccggcctgtccttctaccaacagagaaaggaacccgtgtctgatatcctgaagctgaaaacccacctgcctaccttcagccaggaggacctgatcgttaacgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccacactcgagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 93 - DNA, 9409 bp, synthetic construct; identical to SEQ ID NO: 88
  source 1..9409: mol_type = other DNA

SEQ ID NO: 94 - DNA, 9843 bp, synthetic construct; identical to SEQ ID NO: 89
  source 1..9843: mol_type = other DNA

SEQ ID NO: 95 - DNA, 11262 bp, synthetic construct
  source 1..11262: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttaagattatagttgtcatttgaacttggggataaaggagacatctatgacttggctggaaaagacagagctaatgtacattgcaaagcacatatttatagcaggaaaatgggaagatttctctttaattctggagatggagtggggatggggagagtagactactcattttaagggtgaaacattggaattcaacttgtttgatgttatattaattggtggttaattactaagctaagtacgtataaaacttttatctatggctagcttgtccccccaaagtcatgcaatatagtgaactggctttcgcactttaaattattcattgatcatgtaatgattcagatgattcatcttccaagatggacactgaaactaacactcatagtaggttgtggtttaaagagtggaacaaccgccagtctcattagtggaaattgtgatggttgaatttatcaaggatgaacatacacggtcttctttctgagattttctttaagattttcgcacagataatctatttcttaggttttggagagaaaacttgaattttattgatccctcagaactcaatctttcagatttcaaaggagctatttcttttaatggggactctgttaatatttataaaagctcttcacaggatggagggtgggagggaaactccatcccaacaagacaaaaagaatgaagcatgaggctccacctagttcatcactgctccttgaaatacatcagtattgaaagacacatccaccccacccccaacccagccctattgctgttccagctcaagagtcagaggtcccgaagctgtagctcttctacaatctgctgctctgtgacttcaagtctgttgtctgcaaagaaaactattgggttcccaagcaagagaggcacatctggtaggacagattttgtgattgcaaaagaagggggaaaaaaagaaagaaagaaaagacctctctatacaagataaccagaggcatcaaactgaaatcctcctgtggaaaataagctagtacttctgggcctgatggtgtagtgaaaacctgtgcttgaggatacattacagtgaaagagcaaagtgaatagtaagtagctattacttacctccttagggaggtgtgttgtttgtctgtacatcccccacagcacctagcacagtaccttgcatctcacctgccactcactaaaaagtctatcaagttagttaattatcgagacaacgccctcagaaatgagagaacagtaccctcttatccttgctgcactttccagcactgatacgctgcctaaaagaggactagggcacaggtttgaattaatgtcacaaaactggatgggcaagttacaacggtgttgattaaggaaacagaactcatggtgcaccggatatctccatcctgatgaacccttggaaaaatgccaaagatgcatatccccaggcaaatgcctgattagtctgggattgatagattggtctaggattcagccctactgggaagatgtctaaattataatcagtgtagaaagcgaagttctcctagaagaagaggcaaaggttaaaaagaagaaaagaaaagaaagtgaagtcctttctcccccaaaacctctcatcaatcaatcagggtaacaaacagaacactagggctctgtctgtggaccaaacccaaaagccctgcggtcagggccaggagggtagatcatgtgtttgtggcaacttcctctgtgggcttttgcccaggtctgtccccaagcatacgatggccaaaacttctgcaccagagcagcatcctgtgtaacacagtcaggtccagcagttagggaaaactgcccactcagagtagataatatctggaaggaatgactgtttgggaaaagttccaatgctagttcagtgccaacccttccccaccttctccagctctctcccactggttcctcccctctcaactgctctggttcttataaaaacctcacagccttccactaacatcccgtaggagcctctctccctactgctgctacacaagaccctgagactgacctgcaggacgaaaccctctggctaactagagaacccactgcttactggcttatcgaaattaatacgactcactatagggagacccaagctggctagcgccaccatgaagtgggtaacctttatttcccttctttttctctttagctcggcttattccaggggtgtgtttcgtcgagactacaaagacgatgacgacaagagcgctggcctgaagccaagctgcgccaaggaggtgaagagctgcaagggcagatgtttcgaacggaccttcggcaactgcagatgcgacgccgcctgcgtggaactgggaaactgttgtctggactaccaggagacatgtatcgagcctgagcacatctggacatgcaacaagttcagatgtggcgagaaaagactgactcggagtctgtgcgcctgttctgacgactgcaaggacaagggcgattgctgcatcaactacagcagcgtgtgccagggcgaaaagagctgggtcgaggaaccctgcgagagcatcaacgagccccagtgccctgccggatttgagacacctcctaccctgctcttctccctggatggcttcagagctgagtacctgcatacatggggaggactgctgcctgtgatcagcaagctgaagaagtgcggcacctacacaaagaacatgcggcctgtctaccctaccaagaccttccccaaccactacagcatcgtgaccggcctgtaccccgagagccacggcatcatcgacaacaagatgtacgaccccaaaatgaacgccagcttcagcctgaaatctaaggaaaagttcaaccctgagtggtataagggcgagccaatctgggtgacagccaagtaccagggcctgaagagcggcaccttcttctggcctggcagcgacgtggagatcaacggaatcttccctgatatctacaagatgtataacggctccgtgcctttcgaggagcggatcttggctgttcttcagtggctgcagctgcccaaggacgagagaccccacttctacaccctgtacctggaagaacctgacagcagcggccacagctacggccctgtgtctagcgaggtgatcaaggccctgcaaagagtggatggcatggtgggcatgctgatggatggactgaaggaactgaacctccatcgttgcctgaacctgatcctgatctccgaccacggcatggaacagggcagctgtaaaaagtacatctacctgaacaagtacctgggcgacgtgaagaatatcaaggtgatctacgggccggccgccaggctgcggcctagcgacgtgcccgacaagtattacagcttcaactacgagggcatcgccagaaacctgtcttgcagagaacccaatcaacacttcaagccttacctgaagcactttctgcctaagagactgcacttcgccaaaagcgacagaattgaacccctgacattctatctggaccctcagtggcagctggccctcaatccttctgaacgcaaatactgcggtagcggatttcacggctctgataacgtgttttctaacatgcaggccctgtttgtgggctacggccctggcttcaagcacggaatcgaggctgacaccttcgagaacatcgaagtttacaatctgatgtgcgacctgctgaatctgaccccagcccctaacaacggcacacacggctctctgaatcacctgctcaagaaccccgtgtatacccccaaacaccctaaagaggtccatccactggtgcagtgcccattcaccagaaaccctcgggacaaccttggctgcagctgcaaccctagcatcctccctatcgaagatttccagacccagtttaacctgactgtcgccgaagagaagatcatcaagcacgaaaccctgccctacgggaggcccagagtgctgcagaaagaaaacacaatctgtctgctgagccagcaccagtttatgagcggctacagtcaggacatcctgatgcctctgtggaccagctacaccgtggaccggaatgacagcttcagcaccgaggacttcagcaactgcctgtatcaggacttccgcatccctctgtcccctgtgcataagtgcagcttttacaagaacaacaccaaggtgagctacgggtttctgagcccaccacaactgaacaaaaatagctctggcatctactctgaggcgctgctgaccaccaacatcgtgccaatgtaccagagcttccaggtgatctggcggtacttccatgataccctgcttcggaagtacgccgaagaacggaacggcgtgaacgtggtgtccggcccagtgttcgacttcgactacgacggcagatgtgattcccttgagaatctgagacagaagcggcgggttatcagaaaccaggagatcctgattcctacccacttcttcatcgtgctgacatcttgcaaggataccagccaaacacctctgcactgcgagaacctggacaccctggccttcatcctgcctcaccggaccgataatagcgaatcctgtgtgcacggcaaacacgactctagctgggtggaagagctgctgatgctgcacagagcccggatcacagatgtggaacacatcaccggcctgtccttctaccaacagagaaaggaacccgtgtctgatatcctgaagctgaaaacccacctgcctaccttcagccaggaggacctgatcgttaacgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccacactcgagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 96 - DNA, 10549 bp, synthetic construct; identical to SEQ ID NO: 90
  source 1..10549: mol_type = other DNA

SEQ ID NO: 97 - DNA, 11247 bp, synthetic construct
  source 1..11247: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttaagattatagttgtcatttgaacttggggataaaggagacatctatgacttggctggaaaagacagagctaatgtacattgcaaagcacatatttatagcaggaaaatgggaagatttctctttaattctggagatggagtggggatggggagagtagactactcattttaagggtgaaacattggaattcaacttgtttgatgttatattaattggtggttaattactaagctaagtacgtataaaacttttatctatggctagcttgtccccccaaagtcatgcaatatagtgaactggctttcgcactttaaattattcattgatcatgtaatgattcagatgattcatcttccaagatggacactgaaactaacactcatagtaggttgtggtttaaagagtggaacaaccgccagtctcattagtggaaattgtgatggttgaatttatcaaggatgaacatacacggtcttctttctgagattttctttaagattttcgcacagataatctatttcttaggttttggagagaaaacttgaattttattgatccctcagaactcaatctttcagatttcaaaggagctatttcttttaatggggactctgttaatatttataaaagctcttcacaggatggagggtgggagggaaactccatcccaacaagacaaaaagaatgaagcatgaggctccacctagttcatcactgctccttgaaatacatcagtattgaaagacacatccaccccacccccaacccagccctattgctgttccagctcaagagtcagaggtcccgaagctgtagctcttctacaatctgctgctctgtgacttcaagtctgttgtctgcaaagaaaactattgggttcccaagcaagagaggcacatctggtaggacagattttgtgattgcaaaagaagggggaaaaaaagaaagaaagaaaagacctctctatacaagataaccagaggcatcaaactgaaatcctcctgtggaaaataagctagtacttctgggcctgatggtgtagtgaaaacctgtgcttgaggatacattacagtgaaagagcaaagtgaatagtaagtagctattacttacctccttagggaggtgtgttgtttgtctgtacatcccccacagcacctagcacagtaccttgcatctcacctgccactcactaaaaagtctatcaagttagttaattatcgagacaacgccctcagaaatgagagaacagtaccctcttatccttgctgcactttccagcactgatacgctgcctaaaagaggactagggcacaggtttgaattaatgtcacaaaactggatgggcaagttacaacggtgttgattaaggaaacagaactcatggtgcaccggatatctccatcctgatgaacccttggaaaaatgccaaagatgcatatccccaggcaaatgcctgattagtctgggattgatagattggtctaggattcagccctactgggaagatgtctaaattataatcagtgtagaaagcgaagttctcctagaagaagaggcaaaggttaaaaagaagaaaagaaaagaaagtgaagtcctttctcccccaaaacctctcatcaatcaatcagggtaacaaacagaacactagggctctgtctgtggaccaaacccaaaagccctgcggtcagggccaggagggtagatcatgtgtttgtggcaacttcctctgtgggcttttgcccaggtctgtccccaagcatacgatggccaaaacttctgcaccagagcagcatcctgtgtaacacagtcaggtccagcagttagggaaaactgcccactcagagtagataatatctggaaggaatgactgtttgggaaaagttccaatgctagttcagtgccaacccttccccaccttctccagctctctcccactggttcctcccctctcaactgctctggttcttataaaaacctcacagccttccactaacatcccgtaggagcctctctccctactgctgctacacaagaccctgagactgacctgcaggacgaaaccctctggctaactagagaacccactgcttactggcttatcgaaattaatacgactcactatagggagacccaagctggctagcgccaccatgaagagcctgatccttcttgccatcctggccgccttagcggtagtaactttgtgtgactacaaagacgatgacgacaagagcgctggcctgaagccaagctgcgccaaggaggtgaagagctgcaagggcagatgtttcgaacggaccttcggcaactgcagatgcgacgccgcctgcgtggaactgggaaactgttgtctggactaccaggagacatgtatcgagcctgagcacatctggacatgcaacaagttcagatgtggcgagaaaagactgactcggagtctgtgcgcctgttctgacgactgcaaggacaagggcgattgctgcatcaactacagcagcgtgtgccagggcgaaaagagctgggtcgaggaaccctgcgagagcatcaacgagccccagtgccctgccggatttgagacacctcctaccctgctcttctccctggatggcttcagagctgagtacctgcatacatggggaggactgctgcctgtgatcagcaagctgaagaagtgcggcacctacacaaagaacatgcggcctgtctaccctaccaagaccttccccaaccactacagcatcgtgaccggcctgtaccccgagagccacggcatcatcgacaacaagatgtacgaccccaaaatgaacgccagcttcagcctgaaatctaaggaaaagttcaaccctgagtggtataagggcgagccaatctgggtgacagccaagtaccagggcctgaagagcggcaccttcttctggcctggcagcgacgtggagatcaacggaatcttccctgatatctacaagatgtataacggctccgtgcctttcgaggagcggatcttggctgttcttcagtggctgcagctgcccaaggacgagagaccccacttctacaccctgtacctggaagaacctgacagcagcggccacagctacggccctgtgtctagcgaggtgatcaaggccctgcaaagagtggatggcatggtgggcatgctgatggatggactgaaggaactgaacctccatcgttgcctgaacctgatcctgatctccgaccacggcatggaacagggcagctgtaaaaagtacatctacctgaacaagtacctgggcgacgtgaagaatatcaaggtgatctacgggccggccgccaggctgcggcctagcgacgtgcccgacaagtattacagcttcaactacgagggcatcgccagaaacctgtcttgcagagaacccaatcaacacttcaagccttacctgaagcactttctgcctaagagactgcacttcgccaaaagcgacagaattgaacccctgacattctatctggaccctcagtggcagctggccctcaatccttctgaacgcaaatactgcggtagcggatttcacggctctgataacgtgttttctaacatgcaggccctgtttgtgggctacggccctggcttcaagcacggaatcgaggctgacaccttcgagaacatcgaagtttacaatctgatgtgcgacctgctgaatctgaccccagcccctaacaacggcacacacggctctctgaatcacctgctcaagaaccccgtgtatacccccaaacaccctaaagaggtccatccactggtgcagtgcccattcaccagaaaccctcgggacaaccttggctgcagctgcaaccctagcatcctccctatcgaagatttccagacccagtttaacctgactgtcgccgaagagaagatcatcaagcacgaaaccctgccctacgggaggcccagagtgctgcagaaagaaaacacaatctgtctgctgagccagcaccagtttatgagcggctacagtcaggacatcctgatgcctctgtggaccagctacaccgtggaccggaatgacagcttcagcaccgaggacttcagcaactgcctgtatcaggacttccgcatccctctgtcccctgtgcataagtgcagcttttacaagaacaacaccaaggtgagctacgggtttctgagcccaccacaactgaacaaaaatagctctggcatctactctgaggcgctgctgaccaccaacatcgtgccaatgtaccagagcttccaggtgatctggcggtacttccatgataccctgcttcggaagtacgccgaagaacggaacggcgtgaacgtggtgtccggcccagtgttcgacttcgactacgacggcagatgtgattcccttgagaatctgagacagaagcggcgggttatcagaaaccaggagatcctgattcctacccacttcttcatcgtgctgacatcttgcaaggataccagccaaacacctctgcactgcgagaacctggacaccctggccttcatcctgcctcaccggaccgataatagcgaatcctgtgtgcacggcaaacacgactctagctgggtggaagagctgctgatgctgcacagagcccggatcacagatgtggaacacatcaccggcctgtccttctaccaacagagaaaggaacccgtgtctgatatcctgaagctgaaaacccacctgcctaccttcagccaggaggacctgatcgttaacgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccacactcgagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 98 - DNA, 9843 bp, synthetic construct
  source 1..9843: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgctctggctaactagagaacccactgcttactggcttatcgaaattaatacgactcactatagggagacccaagctggctagcgccaccatgaagtgggtaacctttatttcccttctttttctctttagctcggcttattccaggggtgtgtttcgtcgagactacaaagacgatgacgacaagagcgctggcctgaagccaagctgcgccaaggaggtgaagagctgcaagggcagatgtttcgaacggaccttcggcaactgcagatgcgacgccgcctgcgtggaactgggaaactgttgtctggactaccaggagacatgtatcgagcctgagcacatctggacatgcaacaagttcagatgtggcgagaaaagactgactcggagtctgtgcgcctgttctgacgactgcaaggacaagggcgattgctgcatcaactacagcagcgtgtgccagggcgaaaagagctgggtcgaggaaccctgcgagagcatcaacgagccccagtgccctgccggatttgagacacctcctaccctgctcttctccctggatggcttcagagctgagtacctgcatacatggggaggactgctgcctgtgatcagcaagctgaagaagtgcggcacctacacaaagaacatgcggcctgtctaccctaccaagaccttccccaaccactacagcatcgtgaccggcctgtaccccgagagccacggcatcatcgacaacaagatgtacgaccccaaaatgaacgccagcttcagcctgaaatctaaggaaaagttcaaccctgagtggtataagggcgagccaatctgggtgacagccaagtaccagggcctgaagagcggcaccttcttctggcctggcagcgacgtggagatcaacggaatcttccctgatatctacaagatgtataacggctccgtgcctttcgaggagcggatcttggctgttcttcagtggctgcagctgcccaaggacgagagaccccacttctacaccctgtacctggaagaacctgacagcagcggccacagctacggccctgtgtctagcgaggtgatcaaggccctgcaaagagtggatggcatggtgggcatgctgatggatggactgaaggaactgaacctccatcgttgcctgaacctgatcctgatctccgaccacggcatggaacagggcagctgtaaaaagtacatctacctgaacaagtacctgggcgacgtgaagaatatcaaggtgatctacgggccggccgccaggctgcggcctagcgacgtgcccgacaagtattacagcttcaactacgagggcatcgccagaaacctgtcttgcagagaacccaatcaacacttcaagccttacctgaagcactttctgcctaagagactgcacttcgccaaaagcgacagaattgaacccctgacattctatctggaccctcagtggcagctggccctcaatccttctgaacgcaaatactgcggtagcggatttcacggctctgataacgtgttttctaacatgcaggccctgtttgtgggctacggccctggcttcaagcacggaatcgaggctgacaccttcgagaacatcgaagtttacaatctgatgtgcgacctgctgaatctgaccccagcccctaacaacggcacacacggctctctgaatcacctgctcaagaaccccgtgtatacccccaaacaccctaaagaggtccatccactggtgcagtgcccattcaccagaaaccctcgggacaaccttggctgcagctgcaaccctagcatcctccctatcgaagatttccagacccagtttaacctgactgtcgccgaagagaagatcatcaagcacgaaaccctgccctacgggaggcccagagtgctgcagaaagaaaacacaatctgtctgctgagccagcaccagtttatgagcggctacagtcaggacatcctgatgcctctgtggaccagctacaccgtggaccggaatgacagcttcagcaccgaggacttcagcaactgcctgtatcaggacttccgcatccctctgtcccctgtgcataagtgcagcttttacaagaacaacaccaaggtgagctacgggtttctgagcccaccacaactgaacaaaaatagctctggcatctactctgaggcgctgctgaccaccaacatcgtgccaatgtaccagagcttccaggtgatctggcggtacttccatgataccctgcttcggaagtacgccgaagaacggaacggcgtgaacgtggtgtccggcccagtgttcgacttcgactacgacggcagatgtgattcccttgagaatctgagacagaagcggcgggttatcagaaaccaggagatcctgattcctacccacttcttcatcgtgctgacatcttgcaaggataccagccaaacacctctgcactgcgagaacctggacaccctggccttcatcctgcctcaccggaccgataatagcgaatcctgtgtgcacggcaaacacgactctagctgggtggaagagctgctgatgctgcacagagcccggatcacagatgtggaacacatcaccggcctgtccttctaccaacagagaaaggaacccgtgtctgatatcctgaagctgaaaacccacctgcctaccttcagccaggaggacctgatcgttaacgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccacactcgagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatgacaattacgattagcactatccaattacgattagcactatccaattacgattagcactatcggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 99 - DNA, 9537 bp, synthetic construct
  source 1..9537: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttctagatgtacactcgaggtgagccccacgttctgcttcactctccccatctcccccccctccccacccccaattttgtatttatttattttttaattattttgtgcagcgatgggggcgggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggccaccccgcgggcccgttaattaaaccggtcgccaccatgaagtgggtaacctttatttcccttctttttctctttagctcggcttattccaggggtgtgtttcgtcgagactacaaagacgatgacgacaagagcgctggcctgaagccaagctgcgccaaggaggtgaagagctgcaagggcagatgtttcgaacggaccttcggcaactgcagatgcgacgccgcctgcgtggaactgggaaactgttgtctggactaccaggagacatgtatcgagcctgagcacatctggacatgcaacaagttcagatgtggcgagaaaagactgactcggagtctgtgcgcctgttctgacgactgcaaggacaagggcgattgctgcatcaactacagcagcgtgtgccagggcgaaaagagctgggtcgaggaaccctgcgagagcatcaacgagccccagtgccctgccggatttgagacacctcctaccctgctcttctccctggatggcttcagagctgagtacctgcatacatggggaggactgctgcctgtgatcagcaagctgaagaagtgcggcacctacacaaagaacatgcggcctgtctaccctaccaagaccttccccaaccactacagcatcgtgaccggcctgtaccccgagagccacggcatcatcgacaacaagatgtacgaccccaaaatgaacgccagcttcagcctgaaatctaaggaaaagttcaaccctgagtggtataagggcgagccaatctgggtgacagccaagtaccagggcctgaagagcggcaccttcttctggcctggcagcgacgtggagatcaacggaatcttccctgatatctacaagatgtataacggctccgtgcctttcgaggagcggatcttggctgttcttcagtggctgcagctgcccaaggacgagagaccccacttctacaccctgtacctggaagaacctgacagcagcggccacagctacggccctgtgtctagcgaggtgatcaaggccctgcaaagagtggatggcatggtgggcatgctgatggatggactgaaggaactgaacctccatcgttgcctgaacctgatcctgatctccgaccacggcatggaacagggcagctgtaaaaagtacatctacctgaacaagtacctgggcgacgtgaagaatatcaaggtgatctacgggccggccgccaggctgcggcctagcgacgtgcccgacaagtattacagcttcaactacgagggcatcgccagaaacctgtcttgcagagaacccaatcaacacttcaagccttacctgaagcactttctgcctaagagactgcacttcgccaaaagcgacagaattgaacccctgacattctatctggaccctcagtggcagctggccctcaatccttctgaacgcaaatactgcggtagcggatttcacggctctgataacgtgttttctaacatgcaggccctgtttgtgggctacggccctggcttcaagcacggaatcgaggctgacaccttcgagaacatcgaagtttacaatctgatgtgcgacctgctgaatctgaccccagcccctaacaacggcacacacggctctctgaatcacctgctcaagaaccccgtgtatacccccaaacaccctaaagaggtccatccactggtgcagtgcccattcaccagaaaccctcgggacaaccttggctgcagctgcaaccctagcatcctccctatcgaagatttccagacccagtttaacctgactgtcgccgaagagaagatcatcaagcacgaaaccctgccctacgggaggcccagagtgctgcagaaagaaaacacaatctgtctgctgagccagcaccagtttatgagcggctacagtcaggacatcctgatgcctctgtggaccagctacaccgtggaccggaatgacagcttcagcaccgaggacttcagcaactgcctgtatcaggacttccgcatccctctgtcccctgtgcataagtgcagcttttacaagaacaacaccaaggtgagctacgggtttctgagcccaccacaactgaacaaaaatagctctggcatctactctgaggcgctgctgaccaccaacatcgtgccaatgtaccagagcttccaggtgatctggcggtacttccatgataccctgcttcggaagtacgccgaagaacggaacggcgtgaacgtggtgtccggcccagtgttcgacttcgactacgacggcagatgtgattcccttgagaatctgagacagaagcggcgggttatcagaaaccaggagatcctgattcctacccacttcttcatcgtgctgacatcttgcaaggataccagccaaacacctctgcactgcgagaacctggacaccctggccttcatcctgcctcaccggaccgataatagcgaatcctgtgtgcacggcaaacacgactctagctgggtggaagagctgctgatgctgcacagagcccggatcacagatgtggaacacatcaccggcctgtccttctaccaacagagaaaggaacccgtgtctgatatcctgaagctgaaaacccacctgcctaccttcagccaggaggacctgatcgttaacgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccacactcgagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataaggcgcgcccacgtgtaaaacttgtttattgcagcttataatggttacaaataaagcaatagcatcacaaatttcacaaataaagcatttttttcactgcattctagttgtggtttgtccaaactcatcaatgtatcttatcatgtctggatcatcgatgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 100 - DNA, 9416 bp, synthetic construct
  source 1..9416: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgaaaactacaattgactatctctaaattttaaaatgtgatgatagttactacttcagtggtccacactgtttctatcctttggaacataagattttgttgtgtagcttaaggaagaggaattcagcagttgactatctcgattgttctgggaattttggaggctgaggggaaattttcacatataagcgctgggtttgctggggtattgagagtgacctctggctaactagagaacccactgcttactggcttatcgaaattaatacgactcactatagggagacccaagctggctagcgccaccatgaagtgggtaacctttatttcccttctttttctctttagctcggcttattccaggggtgtgtttcgtcgagactacaaagacgatgacgacaagagcgctggcctgaagccaagctgcgccaaggaggtgaagagctgcaagggcagatgtttcgaacggaccttcggcaactgcagatgcgacgccgcctgcgtggaactgggaaactgttgtctggactaccaggagacatgtatcgagcctgagcacatctggacatgcaacaagttcagatgtggcgagaaaagactgactcggagtctgtgcgcctgttctgacgactgcaaggacaagggcgattgctgcatcaactacagcagcgtgtgccagggcgaaaagagctgggtcgaggaaccctgcgagagcatcaacgagccccagtgccctgccggatttgagacacctcctaccctgctcttctccctggatggcttcagagctgagtacctgcatacatggggaggactgctgcctgtgatcagcaagctgaagaagtgcggcacctacacaaagaacatgcggcctgtctaccctaccaagaccttccccaaccactacagcatcgtgaccggcctgtaccccgagagccacggcatcatcgacaacaagatgtacgaccccaaaatgaacgccagcttcagcctgaaatctaaggaaaagttcaaccctgagtggtataagggcgagccaatctgggtgacagccaagtaccagggcctgaagagcggcaccttcttctggcctggcagcgacgtggagatcaacggaatcttccctgatatctacaagatgtataacggctccgtgcctttcgaggagcggatcttggctgttcttcagtggctgcagctgcccaaggacgagagaccccacttctacaccctgtacctggaagaacctgacagcagcggccacagctacggccctgtgtctagcgaggtgatcaaggccctgcaaagagtggatggcatggtgggcatgctgatggatggactgaaggaactgaacctccatcgttgcctgaacctgatcctgatctccgaccacggcatggaacagggcagctgtaaaaagtacatctacctgaacaagtacctgggcgacgtgaagaatatcaaggtgatctacgggccggccgccaggctgcggcctagcgacgtgcccgacaagtattacagcttcaactacgagggcatcgccagaaacctgtcttgcagagaacccaatcaacacttcaagccttacctgaagcactttctgcctaagagactgcacttcgccaaaagcgacagaattgaacccctgacattctatctggaccctcagtggcagctggccctcaatccttctgaacgcaaatactgcggtagcggatttcacggctctgataacgtgttttctaacatgcaggccctgtttgtgggctacggccctggcttcaagcacggaatcgaggctgacaccttcgagaacatcgaagtttacaatctgatgtgcgacctgctgaatctgaccccagcccctaacaacggcacacacggctctctgaatcacctgctcaagaaccccgtgtatacccccaaacaccctaaagaggtccatccactggtgcagtgcccattcaccagaaaccctcgggacaaccttggctgcagctgcaaccctagcatcctccctatcgaagatttccagacccagtttaacctgactgtcgccgaagagaagatcatcaagcacgaaaccctgccctacgggaggcccagagtgctgcagaaagaaaacacaatctgtctgctgagccagcaccagtttatgagcggctacagtcaggacatcctgatgcctctgtggaccagctacaccgtggaccggaatgacagcttcagcaccgaggacttcagcaactgcctgtatcaggacttccgcatccctctgtcccctgtgcataagtgcagcttttacaagaacaacaccaaggtgagctacgggtttctgagcccaccacaactgaacaaaaatagctctggcatctactctgaggcgctgctgaccaccaacatcgtgccaatgtaccagagcttccaggtgatctggcggtacttccatgataccctgcttcggaagtacgccgaagaacggaacggcgtgaacgtggtgtccggcccagtgttcgacttcgactacgacggcagatgtgattcccttgagaatctgagacagaagcggcgggttatcagaaaccaggagatcctgattcctacccacttcttcatcgtgctgacatcttgcaaggataccagccaaacacctctgcactgcgagaacctggacaccctggccttcatcctgcctcaccggaccgataatagcgaatcctgtgtgcacggcaaacacgactctagctgggtggaagagctgctgatgctgcacagagcccggatcacagatgtggaacacatcaccggcctgtccttctaccaacagagaaaggaacccgtgtctgatatcctgaagctgaaaacccacctgcctaccttcagccaggaggacctgatcgttaacgatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccacactcgagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttatgaggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 101 - DNA, 7499 bp, synthetic construct
  source 1..7499: mol_type = other DNA
ccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccaggctgcaggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaaggggttcaagcttagcgccaccatggagcgcgacggctgcgcggggggcgggagccgcggcggcgagggcgggcgcgctccccgggagggcccggcggggaacggccgcgatcggggccgcagccacgctgccgaggcgcccggggacccgcaggccgcggcgtccttgctggcccctatggacgtgggggaggagccgctggagaaggcggcgcgcgcccgcactgccaaggaccccaacacctataaagtactctcgctggtattgtcagtatgtgtgttaacaacaatacttggttgtatatttgggttgaaaccaagctgtgccaaagaagttaaaagttgcaaaggtcgctgtttcgagagaacatttgggaactgtcgctgtgatgctgcctgtgttgagcttggaaactgctgtttagattaccaggagacgtgcatagaaccagaacatatatggacttgcaacaaattcaggtgtggtgagaaaaggttgaccagaagcctctgtgcctgttcagatgactgcaaggacaagggcgactgctgcatcaactacagttctgtgtgtcaaggtgagaaaagttgggtagaagaaccatgtgagagcattaatgagccacagtgcccagcagggtttgaaacgcctcctaccctcttattttctttggatggattcagggcagaatatttacacacttggggtggacttcttcctgttattagcaaactaaaaaaatgtggaacatatactaaaaacatgagaccggtatatccaacaaaaactttccccaatcactacagcattgtcaccggattgtatccagaatctcatggcataatcgacaataaaatgtatgatcccaaaatgaatgcttccttttcacttaaaagtaaagagaaatttaatcctgagtggtacaaaggagaaccaatttgggtcacagctaagtatcaaggcctcaagtctggcacatttttctggccaggatcagatgtggaaattaacggaattttcccagacatctataaaatgtataatggttcagtaccatttgaagaaaggattttagctgttcttcagtggctacagcttcctaaagatgaaagaccacacttttacactctgtatttagaagaaccagattcttcaggtcattcatatggaccagtcagcagtgaagtcatcaaagccttgcagagggttgatggtatggttggtatgctgatggatggtctgaaagagctgaacttgcacagatgcctgaacctcatccttatttcagatcatggcatggaacaaggcagttgtaagaaatacatatatctgaataaatatttgggggatgttaaaaatattaaagttatctatggacctgcagctcgattgagaccctctgatgtcccagataaatactattcatttaactatgaaggcattgcccgaaatctttcttgccgggaaccaaaccagcacttcaaaccttacctgaaacatttcttacctaagcgtttgcactttgctaagagtgatagaattgagcccttgacattctatttggaccctcagtggcaacttgcattgaatccctcagaaaggaaatattgtggaagtggatttcatggctctgacaatgtattttcaaatatgcaagccctctttgttggctatggacctggattcaagcatggcattgaggctgacacctttgaaaacattgaagtctataacttaatgtgtgatttactgaatttgacaccggctcctaataacggaactcatggaagtcttaaccaccttctaaagaatcctgtttatacgccaaagcatcccaaagaagtgcaccccctggtacagtgccccttcacaagaaaccccagagataaccttggctgctcatgtaacccttcgattttgccgattgaggattttcaaacacagttcaatctgactgtggcagaagagaagattattaagcatgaaactttaccctatggaagacctagagttctccagaaggaaaacaccatctgtcttctttcccagcaccagtttatgagtggatacagccaagacatcttaatgcccctttggacatcctataccgtggacagaaatgacagtttctctacggaagacttctccaactgtctgtaccaggactttagaattcctcttagtcctgtccataaatgttcattttataaaaataacaccaaagtgagttacgggttcctctccccaccacaactaaataaaaattcaagtggaatatattctgaagctttgcttactacaaatatagtgccaatgtaccagagttttcaagttatatggcgctactttcatgacaccctactgcgaaagtatgctgaagaaagaaatggtgtcaatgtcgtcagtggtcctgtgtttgactttgattatgatggacgttgtgattccttagagaatctgaggcaaaaaagaagagtcatccgtaaccaagaaattttgattccaactcacttctttattgtgctaacaagctgtaaagatacatctcagacgcctttgcactgtgaaaacctagacaccttagctttcattttgcctcacaggactgataacagcgagagctgtgtgcatgggaagcatgactcctcatgggttgaagaattgttaatgttacacagagcacggatcacagatgttgagcacatcactggactcagcttctatcaacaaagaaaagagccagtttcagacattttaaagttgaaaacacatttgccaacctttagccaagaagacgactacaaagacgatgacgacaagtgaggtaccgagctcggatccaaacaaacaccattgtcacactccaacaaacaccattgtcacactccaacaaacaccattgtcacactccattctcgagtctagaaagagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaa

SEQ ID NO: 102 - DNA, 7487 bp, synthetic construct
  source 1..7487: mol_type = other DNA
ccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccaggctgcaggggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaaggggttcaagcttagcgccaccatggagcgcgacggctgcgcggggggcgggagccgcggcggcgagggcgggcgcgctccccgggagggcccggcggggaacggccgcgatcggggccgcagccacgctgccgaggcgcccggggacccgcaggccgcggcgtccttgctggcccctatggacgtgggggaggagccgctggagaaggcggcgcgcgcccgcactgccaaggaccccaacacctataaagtactctcgctggtattgtcagtatgtgtgttaacaacaatacttggttgtatatttgggttgaaaccaagctgtgccaaagaagttaaaagttgcaaaggtcgctgtttcgagagaacatttgggaactgtcgctgtgatgctgcctgtgttgagcttggaaactgctgtttagattaccaggagacgtgcatagaaccagaacatatatggacttgcaacaaattcaggtgtggtgagaaaaggttgaccagaagcctctgtgcctgttcagatgactgcaaggacaagggcgactgctgcatcaactacagttctgtgtgtcaaggtgagaaaagttgggtagaagaaccatgtgagagcattaatgagccacagtgcccagcagggtttgaaacgcctcctaccctcttattttctttggatggattcagggcagaatatttacacacttggggtggacttcttcctgttattagcaaactaaaaaaatgtggaacatatactaaaaacatgagaccggtatatccaacaaaaactttccccaatcactacagcattgtcaccggattgtatccagaatctcatggcataatcgacaataaaatgtatgatcccaaaatgaatgcttccttttcacttaaaagtaaagagaaatttaatcctgagtggtacaaaggagaaccaatttgggtcacagctaagtatcaaggcctcaagtctggcacatttttctggccaggatcagatgtggaaattaacggaattttcccagacatctataaaatgtataatggttcagtaccatttgaagaaaggattttagctgttcttcagtggctacagcttcctaaagatgaaagaccacacttttacactctgtatttagaagaaccagattcttcaggtcattcatatggaccagtcagcagtgaagtcatcaaagccttgcagagggttgatggtatggttggtatgctgatggatggtctgaaagagctgaacttgcacagatgcctgaacctcatccttatttcagatcatggcatggaacaaggcagttgtaagaaatacatatatctgaataaatatttgggggatgttaaaaatattaaagttatctatggacctgcagctcgattgagaccctctgatgtcccagataaatactattcatttaactatgaaggcattgcccgaaatctttcttgccgggaaccaaaccagcacttcaaaccttacctgaaacatttcttacctaagcgtttgcactttgctaagagtgatagaattgagcccttgacattctatttggaccctcagtggcaacttgcattgaatccctcagaaaggaaatattgtggaagtggatttcatggctctgacaatgtattttcaaatatgcaagccctctttgttggctatggacctggattcaagcatggcattgaggctgacacctttgaaaacattgaagtctataacttaatgtgtgatttactgaatttgacaccggctcctaataacggaactcatggaagtcttaaccaccttctaaagaatcctgtttatacgccaaagcatcccaaagaagtgcaccccctggtacagtgccccttcacaagaaaccccagagataaccttggctgctcatgtaacccttcgattttgccgattgaggattttcaaacacagttcaatctgactgtggcagaagagaagattattaagcatgaaactttaccctatggaagacctagagttctccagaaggaaaacaccatctgtcttctttcccagcaccagtttatgagtggatacagccaagacatcttaatgcccctttggacatcctataccgtggacagaaatgacagtttctctacggaagacttctccaactgtctgtaccaggactttagaattcctcttagtcctgtccataaatgttcattttataaaaataacaccaaagtgagttacgggttcctctccccaccacaactaaataaaaattcaagtggaatatattctgaagctttgcttactacaaatatagtgccaatgtaccagagttttcaagttatatggcgctactttcatgacaccctactgcgaaagtatgctgaagaaagaaatggtgtcaatgtcgtcagtggtcctgtgtttgactttgattatgatggacgttgtgattccttagagaatctgaggcaaaaaagaagagtcatccgtaaccaagaaattttgattccaactcacttctttattgtgctaacaagctgtaaagatacatctcagacgcctttgcactgtgaaaacctagacaccttagctttcattttgcctcacaggactgataacagcgagagctgtgtgcatgggaagcatgactcctcatgggttgaagaattgttaatgttacacagagcacggatcacagatgttgagcacatcactggactcagcttctatcaacaaagaaaagagccagtttcagacattttaaagttgaaaacacatttgccaacctttagccaagaagacgactacaaagacgatgacgacaagtgaggtaccgagctcggatcccaattacgattagcactatccaattacgattagcactatccaattacgattagcactatcctcgagtctagaaagagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccccccccccccccccccctgcagcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaa

SEQ ID NO: 103 - DNA, 7473 bp, synthetic construct
  source 1..7473: mol_type = other DNA
tcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccctgcagcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccaggctgcaggggggggggggggggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaaggggttcaagctctagcgccaccatggagcgcgacggctgcgcggggggcgggagccgcggcggcgagggcgggcgcgctccccgggagggcccggcggggaacggccgcgatcggggccgcagccacgctgccgaggcgcccggggacccgcaggccgcggcgtccttgctggcccctatggacgtgggggaggagccgctggagaaggcggcgcgcgcccgcactgccaaggaccccaacacctataaagtactctcgctggtattgtcagtatgtgtgttaacaacaatacttggttgtatatttgggttgaaaccaagctgtgccaaagaagttaaaagttgcaaaggtcgctgtttcgagagaacatttgggaactgtcgctgtgatgctgcctgtgttgagcttggaaactgctgtttagattaccaggagacgtgcatagaaccagaacatatatggacttgcaacaaattcaggtgtggtgagaaaaggttgaccagaagcctctgtgcctgttcagatgactgcaaggacaagggcgactgctgcatcaactacagttctgtgtgtcaaggtgagaaaagttgggtagaagaaccatgtgagagcattaatgagccacagtgcccagcagggtttgaaacgcctcctaccctcttattttctttggatggattcagggcagaatatttacacacttggggtggacttcttcctgttattagcaaactaaaaaaatgtggaacatatactaaaaacatgagaccggtatatccaacaaaaactttccccaatcactacagcattgtcaccggattgtatccagaatctcatggcataatcgacaataaaatgtatgatcccaaaatgaatgcttccttttcacttaaaagtaaagagaaatttaatcctgagtggtacaaaggagaaccaatttgggtcacagctaagtatcaaggcctcaagtctggcacatttttctggccaggatcagatgtggaaattaacggaattttcccagacatctataaaatgtataatggttcagtaccatttgaagaaaggattttagctgttcttcagtggctacagcttcctaaagatgaaagaccacacttttacactctgtatttagaagaaccagattcttcaggtcattcatatggaccagtcagcagtgaagtcatcaaagccttgcagagggttgatggtatggttggtatgctgatggatggtctgaaagagctgaacttgcacagatgcctgaacctcatccttatttcagatcatggcatggaacaaggcagttgtaagaaatacatatatctgaataaatatttgggggatgttaaaaatattaaagttatctatggacctgcagctcgattgagaccctctgatgtcccagataaatactattcatttaactatgaaggcattgcccgaaatctttcttgccgggaaccaaaccagcacttcaaaccttacctgaaacatttcttacctaagcgtttgcactttgctaagagtgatagaattgagcccttgacattctatttggaccctcagtggcaacttgcattgaatccctcagaaaggaaatattgtggaagtggatttcatggctctgacaatgtattttcaaatatgcaagccctctttgttggctatggacctggattcaagcatggcattgaggctgacacctttgaaaacattgaagtctataacttaatgtgtgatttactgaatttgacaccggctcctaataacggaactcatggaagtcttaaccaccttctaaagaatcctgtttatacgccaaagcatcccaaagaagtgcaccccctggtacagtgccccttcacaagaaaccccagagataaccttggctgctcatgtaacccttcgattttgccgattgaggattttcaaacacagttcaatctgactgtggcagaagagaagattattaagcatgaaactttaccctatggaagacctagagttctccagaaggaaaacaccatctgtcttctttcccagcaccagtttatgagtggatacagccaagacatcttaatgcccctttggacatcctataccgtggacagaaatgacagtttctctacggaagacttctccaactgtctgtaccaggactttagaattcctcttagtcctgtccataaatgttcattttataaaaataacaccaaagtgagttacgggttcctctccccaccacaactaaataaaaattcaagtggaatatattctgaagctttgcttactacaaatatagtgccaatgtaccagagttttcaagttatatggcgctactttcatgacaccctactgcgaaagtatgctgaagaaagaaatggtgtcaatgtcgtcagtggtcctgtgtttgactttgattatgatggacgttgtgattccttagagaatctgaggcaaaaaagaagagtcatccgtaaccaagaaattttgattccaactcacttctttattgtgctaacaagctgtaaagatacatctcagacgcctttgcactgtgaaaacctagacaccttagctttcattttgcctcacaggactgataacagcgagagctgtgtgcatgggaagcatgactcctcatgggttgaagaattgttaatgttacacagagcacggatcacagatgttgagcacatcactggactcagcttctatcaacaaagaaaagagccagtttcagacattttaaagttgaaaacacatttgccaacctttagccaagaagacgactacaaagacgatgacgacaagtgaggtaccgagctcggatccactagtccagtgtggtggaattctgcagatatccagcacagtggcggccgctcgagtctagaaagagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttag

SEQ ID NO: 104 - DNA, 12 bp, synthetic construct
  source 1..12: mol_type = other DNA
ctgatcgttaac

SEQ ID NO: 105 - DNA, 23 bp, synthetic construct
  source 1..23: mol_type = other DNA
tgtttgtggtaacagtgtgaggt

SEQ ID NO: 106 - DNA, 23 bp, synthetic construct
  source 1..23: mol_type = other DNA
acaaacaccattgtcacactcca